SEQUENCE LISTING (78 sequences)

SEQ ID NO: 1 - DNA, 305 bp, synthetic construct
  misc_feature 1..305: note = Description of Artificial Sequence: Synthetic polynucleotide
  source 1..305: mol_type = other DNA
tccttcctttctaaatgacgagagagacagaagaattcttcaaggttagtgtgtccagcatgcaacctttccttcctggatgagcatccctggagtaggagagccagcctgcctcctgcgctggcacagagcccggttccctagacaactgcctctccaaatctgatgtccagcgccacctggtgtccacatcaagcagacacaattaatagtcaacctgttcaggaaaactgtgagggggaaaaaaaagaaagaggatttatgaagggaaaagaaagtttagaggatatgccacgattggctag

SEQ ID NO: 2 - DNA, 1074 bp, synthetic construct
  misc_feature 1..1074: note = Description of Artificial Sequence: Synthetic polynucleotide
  source 1..1074: mol_type = other DNA
aagtaaacttccacaaccgcaagcttattgaggctaaggcatctgtgaaggaaagaaacatctcctctaaaccactatgctgctagagcctcttttctgtactcaagcctcattcagacactagtgtcaccagtctcctcatatacctattgtattttcttcttcttgctggtttagtcatgttttctgggagcttaggggcttattttattttgttttgttttctaatcaacagagatgggcaaacccattatttttttctttagacttgggatggtgatagctgggcagcgtcagaaactgtgtgtggatatagataagagctcggactatgctgagctgtgatgagggagggacctagccaaaggcagtgagagtcagaatgctcctgctattgccttctcagtccccacgcttggtttctacacaagtagatacatagaaaaggctataggttagtgtttgagagtcctgcatgagttagttgctcagaaatgcccgataaatatgttatgtgtgtttatgtatatatatgttttatatatatatatgtgtgtgtgtgtgtgtgtgtgtgttgtgtttacaaatatgtgattatcatcaaaacgtgagggctaaagtgaccagataacttgcaggtcctaggataccaggaaaataaattacattccaaaaatttaactgagactttaaaaaaaaaaaaaaaaaaaaaaaaaaaaccagtgatccatggacacagggaggggaacatcacacactggggcctgttgggggtggggggctaggggaaggatagcattaggagaaatacctaatgtagatgacgggttgatgggtgcagcaaaccaccatggcacatgtaccccagaacttaaagcatattaaaaaaacagtgatcataaaagaagctcaaatttaactataagagacggaatggctcccacaattcttaactataatcttacagaatattctcattgaatagaagtatgcttatcattagagatttggacagccaggaaagcacagaaaaaaaaaaaaggagctctgttgccttatagcctagaggtgttt

SEQ ID NO: 3 - DNA, 602 bp, synthetic construct
  misc_feature 1..602: note = Description of Artificial Sequence: Synthetic polynucleotide
  source 1..602: mol_type = other DNA
ggcatctgtgaaggaaagaaacatctcctctaaaccactatgctgctagagcctcttttctgtactcaagcctcattcagacactagtgtcaccagtctcctcatatacctattgtattttcttcttcttgctggtttagtcatgttttctgggagcttaggggcttattttattttgttttgttttctaatcaacagagatgggcaaacccattatttttttctttagacttgggatggtgatagctgggcagcgtcagaaactgtgtgtggatatagataagagctcggactatgctgagctgtgatgagggagggacctagccaaaggcagtgagagtcagaatgctcctgctattgccttctcagtccccacgcttggtttctacacaagtagatacatagaaaaggctataggttagtgtttgagagtcctgcatgagttagttgctcagaaatgcccgataaatatgttatgtgtgtttatgtatatatatgttttatatatatatatgtgtgtgtgtgtgtgtgtgtgtgttgtgtttacaaatatgtgattatcatcaaaacgtgagggctaaagtgaccagataacttgcagg

SEQ ID NO: 4 - DNA, 489 bp, synthetic construct
  misc_feature 1..489: note = Description of Artificial Sequence: Synthetic polynucleotide
  source 1..489: mol_type = other DNA
ggcatctgtgaaggaaagaaacatctcctctaaaccactatgctgctagagcctcttttctgtactcaagcctcattcagacactagtgtcaccagtctcctcatatacctattgtattttcttcttcttgctggtttagtcatgttttctgggagcttaggggcttattttattttgttttgttttctaatcaacagagatgggcaaacccattatttttttctttagacttgggatggtgatagctgggcagcgtcagaaactgtgtgtggatatagataagagctcggactatgctgagctgtgatgagggagggacctagccaaaggcagtgagagtcagaatgctcctgctattgccttctcagtccccacgcttggtttctacacaagtagatacatagaaaaggctataggttagtgtttgagagtcctgcatgagttagttgctcagaaatgcccgataaatatgttatgtgtgtttatgt

SEQ ID NO: 5 - DNA, 1301 bp, synthetic construct
  misc_feature 1..1301: note = Description of Artificial Sequence: Synthetic polynucleotide
  source 1..1301: mol_type = other DNA
aagctttcattaaaaaaagtctaaccagctgcattcgactttgactgcagcagctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctgagctcagtcttgtcatggcaaaataaagataataatagtgtttttttatggagttagcgtgaggatggaaaacaatagcaaaattgattagactataaaaggtctcaacaaatagtagtagattttatcatccattaatccttccctctcctctcttactcatcccatcacgtatgcctcttaattttcccttacctataataagagttattcctcttattatattcttcttatagtgattctggatattaaagtgggaatgaggggcaggccactaacgaagaagatgtttctcaaagaagccattctccccacatagatcatctcagcagggttcaggaagataaaggaggatcaaggtcgaaggtaggaactaaggaagaacactgggcaagtggatcc

SEQ ID NO: 6 - DNA, 1065 bp, synthetic construct
  misc_feature 1..1065: note = Description of Artificial Sequence: Synthetic polynucleotide
  source 1..1065: mol_type = other DNA
tgagccccttttcctctaactgaaagaaggaaaaaaaaaatggaacccaaaatattctacatagtttccatgtcacagccagggctgggcagtctcctgttatttcttttaaaataaatatatcatttaaatgcataaataagcaaaccctgctcgggaatgggagggagagtctctggagtccaccccttctcggccctggctctgcagatagtgctatcaaagccctgacagagccctgcccattgctgggccttggagtgagtcagcctagtagagaggcagggcaagccatctcatagctgctgagtgggagagagaaaagggctcattgtctataaactcaggtcatggctattcttattctcacactaagaaaaagaatgagatgtctacatataccctgcgtcccctcttgtgtactggggcccccaagagctctctaaaagtgatggcaaagtcattgcgctagatgccatcccatctattataaacctgcatttgtctccacacaccagtcatggacaataaccctcctcccaggtccacgtgcttgtctttgtataatactcaagtaatttcggaaaatgtattctttcaatcttgttctgttattcctgtttcaatggcttagtagaaaaagtacatacttgttttcccataaattgacaatagacaatttcacatcaatgtctatatgggtcgttgtgtttgctgtgtttgcaaaaactcacaataactttatattgttactactctaagaaagttacaacatggtgaatacaagagaaagctattacaagtccagaaaataaaagttatcatcttgaggcctcagctttctaggaataatatcaatattacaaaatttaatctaacaattatgaacagcaatgagataatatgtacaaagtacccagacctatgtggtagagcatcaaggaagcgcattgcggagcagttttttgtttgtttgtttttgtattctgtttcgtgaggcaaggtttcactctgctgtccaggctggagtgcagtggcaagatcatgtctcactgcagccttgac

SEQ ID NO: 7 - DNA, 1065 bp, synthetic construct
  misc_feature 1..1065: note = Description of Artificial Sequence: Synthetic polynucleotide
  source 1..1065: mol_type = other DNA
tgagccccttttcctctaactgaaagaaggaaaaaaaaaatggaacccaaaatattctacatagtttccatgtcacagccagggctgggcagtctcctgttatttcttttaaaataaatatatcattaaatgcataaataagcaaaccctgctcgggaatgggagggagagtctctggagtccaccccttctcggccctggctctgcagatagtgctatcaaagccctgacagagccctgcccattgctgggccttggagtgagtcagcctagtagagaggcagggcaagccatctcatagctgctgagtgggagagagaaaagggctcattgtctataaactcaggtcatggctattcttattctcacactaagaaaaagaatgagatgtctacatataccctgcgtcccctcttgtgtactggggcccccaagagctctctaaaagtgatggcaaagtcattgcgctagatgccatcccatctattataaacctgcatttgtctccacacaccagtcatggacaataaccctcctcccaggtccacgtgcttgtctttgtataatactcaagtaatttcggaaaatgtattctttcaatcttgttctgttattcctgtttcaatggcttagtagaaaaagtacatacttgttttcccataaattgacaatagacaatttcacatcaatgtctatatgggtcgttgtgtttgctgtgtttgcaaaaactcacaataactttatattgttactactctaagaaagttacaacatggtgaatacaagagaaagctattacaagtccagaaaataaaagttatcatcttgaggcctcagctttctaggaataatatcaatattacaaaattaatctaacaattatgaacagcaatgagataatatgtacaaagtacccagacctatgtggtagagcatcaaggaagcgcattgcggagcagttttttgtttgtttgtttttgtattctgtttcgtgaggcaaggtttcactctgctgtccaggctggagtgcagtggcaagatcatgtctcactgcagccttgacac

SEQ ID NO: 8 - DNA, 446 bp, synthetic construct
  misc_feature 1..446: note = Description of Artificial Sequence: Synthetic polynucleotide
  source 1..446: mol_type = other DNA
tggaacccaaaatattctacatagtttccatgtcacagccagggctgggcagtctcctgttatttcttttaaaataaatatatcatttaaatgcataaataagcaaaccctgctcgggaatgggagggagagtctctggagtccaccccttctcggccctggctctgcagatagtgctatcaaagccctgacagagccctgcccattgctgggccttggagtgagtcagcctagtagagaggcagggcaagccatctcatagctgctgagtgggagagagaaaagggctcattgtctataaactcaggtcatggctattcttattctcacactaagaaaaagaatgagatgtctacatataccctgcgtcccctcttgtgtactggggtccccaagagctctctaaaagtgatggcaaagtcattgcgctagatgccatcccatct

SEQ ID NO: 9 - DNA, 860 bp, synthetic construct
  misc_feature 1..860: note = Description of Artificial Sequence: Synthetic polynucleotide
  source 1..860: mol_type = other DNA
gtatatgtgtatatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagcttatttaatcagagatgagatgctggaagggatagagggagctgagcttgtaaaaagtatagtaatcattcagcaaatggttttgaagcacctgctggatgctaaacactattttcagtgcttgaatcataaataagaataaaacatgtatcttattccccacaagagtccaagtaaaaaataacagttaattataatgtgctctgtcccccaggctggagtgcagtggcacgatctcagctcactgcaacctccgcctcccgggttcaagcaattctcctgcctcagccaccctaatagctgggattacaggtgcacaccaccatgccaggctaatttttgtactttttgtagaggcagggtatcaccatgttgtccaagatggtcttgaactcctgagctccaagcagtccacccacctcagcctcccaaagtgct

SEQ ID NO: 10 - DNA, 613 bp, Homo sapiens
  source 1..613: mol_type = genomic DNA
aagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacgtaatatttggaatcacagcttggtaagcatattgaagatcgttttcccaattttcttattacacaaataagaaattgatgcactaaaagtggaagagttttgtctaccataattcagctttgggatatgtagatggatctcttcctgcgtctccagaatatgcaaaatacttacaggacagaatggatgaaaactctacctcagttctaagcatatcttctccttatttggattaaaaccttctggtaagaaaagaaaaaaaatatatatatatatgtgtatatatacacacatacatatacatatatatgcattcatttgttgttgtttttcttaatttgctcatg

SEQ ID NO: 11 - DNA, 265 bp, Homo sapiens
  source 1..265: mol_type = genomic DNA
aagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttac

SEQ ID NO: 12 - DNA, 879 bp, Homo sapiens
  source 1..879: mol_type = genomic DNA
taggtattgaataagaaaaatgaagttaaggtggttgatggtaacactatgctaataactgcagagccagaagcaccataagggacatgataagggagccagcagacctctgatctcttcctgaatgctaatcttaaacatcctgaggaagaatgggacttccatttggggtgggcctatgatagggtaataagacagtagtgaatatcaagctacaaaaagccccctttcaaattcttctcagtcctaacttttcatactaagcccagtccttccaaagcagactgtgaaagagtgatagttccgggagactagcactgcagattccgggtcactgtgagtgggggaggcagggaagaagggctcacaggacagtcaaaccatgccccctgtttttccttcttcaagtagacctctataagacaacagagacaactaaggctgagtggccaggcgaggagaaaccatctcgccgtaaaacatggaaggaacacttcaggggaaaggtggtatctctaagcaagagaactgagtggagtcaaggctgagagatgcaggataagcaaatgggtagtgaaaagacattcatgaggacagctaaaacaataagtaatgtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattg

SEQ ID NO: 13 - DNA, 152 bp, synthetic construct
  misc_feature 1..152: note = Description of Artificial Sequence: Synthetic polynucleotide
  source 1..152: mol_type = other DNA
tctcccacgccctggtctcagcttggggagtggtcagaccccaatggcgataaactctggcaactttatctgtgcactgcaggctcagccccaacagctttagctttcacaagcaggcaggggaagggaaacacatatctccagatatgagg

SEQ ID NO: 14 - DNA, 157 bp, synthetic construct
  misc_feature 1..157: note = Description of Artificial Sequence: Synthetic polynucleotide
  source 1..157: mol_type = other DNA
ctaaacccctcccccaccctagccccaagcttcatcttagctccactcctgaccctatccagctaaaggtccccacccagctcctgcctatctagtcattgcatatggcaagacttgaaagtcctatctcaaagcagcagaattatcagctacgact

SEQ ID NO: 15 - DNA, 141 bp, synthetic construct
  misc_feature 1..141: note = Description of Artificial Sequence: Synthetic polynucleotide
  source 1..141: mol_type = other DNA
ccatcccccagcactccctgcccccacagcccagacttgaccaactcccagctccgcctgggacttccagatatggggccccacccttgcaggccttggggacgctgaagatattgactatctgcgtgccggaaaagggtg

SEQ ID NO: 16 - DNA, 171 bp, synthetic construct
  misc_feature 1..171: note = Description of Artificial Sequence: Synthetic polynucleotide
  source 1..171: mol_type = other DNA
aaaggctgggggtgggagtagcggatttgaagcacttgttggcctacagaggtgtggcaagcagagcacctcagaactcaggcgtactgcccgccgcccgagccctgcgagggccgatagcgagggtgtggcccttatctgcacccagcagagcgccggcggggtacggtc

SEQ ID NO: 17 - DNA, 195 bp, synthetic construct
  misc_feature 1..195: note = Description of Artificial Sequence: Synthetic polynucleotide
  source 1..195: mol_type = other DNA
cagttgcctcagctgagtatgtcttctaaagataatgtcgattgtgtatggctgatgggattctaggaccaagcaagaggtttttttttttcccccacatacttaacgtttctatatttctatttgaattcgactggacagttccatttgaattatttctctctctctctctctctgacacattttatcttgcca

SEQ ID NO: 18 - DNA, 14 bp, synthetic construct
  misc_feature 1..14: note = Description of Artificial Sequence: Synthetic oligonucleotide
  source 1..14: mol_type = other DNA
caccaggtggcgct

SEQ ID NO: 19 - DNA, 81706 bp, Homo sapiens
  source 1..81706: mol_type = genomic DNA
ggatcctcacatgagttcagtatataattgtaacagaataaaaaatcaattatgtattcaagttgctagtgtcttaagaggttcacatttttatctaactgattatcacaaaaatacttcgagttacttttcattataattcctgactacacatgaagagactgacacgtaggtgccttacttaggtaggttaagtaatttatccaaaaccacacaatgtagaacctaagctgattcggccatagaaacacaatatgtggtataaatgagacagagggatttctctccttcctatgctgtcagatgaatactgagatagaatatttagttcatctatcacacattaaacgggactttacatttctgtctgttgaagatttgggtgtggggataactcaaggtatcatatccaagggatggatgaaggcaggtgactctaacagaaagggaaaggatgttggcaaggctatgttcatgaaagtatatgtaaaatccacattaagcttctttctgcatgcattggcaatgtttatgaataatgtgtatgtaaaagtgtgctgtatattcaaaagtgtttcatgtgcctaggggtgtcaaatactttgagtttgtaagtatatacttctctgtaatgtgtctgaatatctctatttacttgattctcaataagtaggtatcatagtgaacatctgacaaatgtttgaggaacaatttagtgtttacctattcaccaaaatttattaaatgcctaatctgtatcagatatacaattatctggcgaaatctgtaattcctaatttaaacagctgtgtagcctaattagggataaaggcatgcaaacccataatttgtgtaggttgaaatgagctatagaaaaatgcagtatatttatcagaagtctttagggtcatgaaaaggaatggtcaactgacactgccagggactcatatgtaagagataactaatgtgaagtgactttaaaggagaaattagcagaagttttctttccatgtctcctcatcatgttacaataacggaagagattaaaacaacaaatacatttagacagcaatgtttatcctggttagatgttttaatctaaatctatcttggagtgttaaaatgcatttgctcacctactttaaaatataaatgaaggtaggaacctgtagatacaaaaagttggagaaaaaaagacaataaagatgacaaaaatctattaatccttgatagaaaatgagaagagataaaacactggtttacataaagaaaataagatggatagatagcagatccttataaaagtgataatttgagaaaaaaaatactccatattctgagtttcttcacataaaataatacaaatctgctgtggtaagttacaaagagatagattttttatcattatataaaagatattttaaacagagttatacaacaaaggaacagactatgtcatatattctcacttatcactataaacatctcagaaaaatctgcaaaatcatttcatagcattttaaatagttaggaataatgtagaaaactgaaacagttctaagtttcccacaaacttagagtctcaaatgttgcattacctaacttacctgcaaatattttatacaaatttgcacatgctactctagtcaaaaatatatgtacattatgggtattttctgtgtgtaacttggttctagttgcttctttcagaaatagcctctatttttgatttacctgataaaatcacattcctctccaaagccttctaaatacttccagactaactactttttagtacatctaagaagaaaagagttttgtctcttatccacctctgagtcaaaaagcagcatgtccatcaattggtacatagttcccacagccccacttagctctggattggagttctacttggcattgtttgcaactacatggacgtaaaatgcatggattctcttgaaaaaatgtttctgccatgatgttctctgaaagagactaaccttccctcgctttgcagagaaagactcgtgtaatccttgacaatgtcatctcatctatttattcccatgtctacccatatgtgaccttcatgtctttgctctaagcccctacatcctcaatctacacactaggatagtataaaagtaatagtaataatagtagtaatagtaataacaatacaatgattatggcttatactatacacaagacactgttgatatattatttcatttagtattcacagtaactctgtgcctcaagtactattgtaataccctttaagaggaggaaactgaggcacagggccctaaagtaatattccaagatgaagtggctactaactgacagagggcataattcaactcatgatatttggctctagaatacatgctctgaatcattatacaataataattcatgaggaaacattttttaaagcctaagttatttgctctgaaataagacataatttggggtgagaaagcttagattccatgaagtattacagcatttggtagtctttttgcactccaggtcttatttttactgcttaaacataataaaacatatggttcagtatgcctttgattttacaataatattcctgttatttttggaagcacagggtgtgggataatgctaattactagtgattagtattgagaggtgacagcgtgctggcagtcctcacagccctcgctcgctcttggcgcctcctctgcctgggctcccacattggtggcacttgaggagcccttcagccggccgctgcactgtgggagcccttttctgggctggccaaggccagagccggctccctcagcttgccaggaggtgtggagggacagacgcgggcaggaaccgggctgtgcgccgtgcttgagggagttccgggtgggcatgggctccgaggaccccgcactcggagccgccagccggccccaccggccgcgggcagtgaggggcttagcacctgggccagcagctgctgtgctcaattcctcgccgggccttagctgccttcctgcggggcagggctcgggacctgcagcgcgccatgcctgagcctccccaccttcatgggctcctgtgcggcccgagcctcgccgacgagcgccgccccctgctccagggcacccagtcccatcgaccacccaagggctgaagagtgcgggcgcacggcaggggactggcaggcagctccccctgcagcccaggtgcgggatccactgggtgaagccggctaggctcctgagtttgctggggatgcgaagaacccttatgtctagataagggattgtaaatacaccaattggcactctgtatctagctcaaggtttgtaaacacaccaatcagcaccctgtgtctagctcagggtttgtgaatgcaccaatcaacactctatctagctactctggtggggccttggagaacctttatgtctagctcagggattgtaaatacaccaatcggcagtctgtatctagctcaaggtttgtaaacacaccaatcagcaccctgtgtctagctcagggtttgtgaatgcaccaatcaacactctgtatctagctactctggtggggacgtggagaacctttatgtctagctcagggattgtaaatacaccactcggcagtctgtatctagctcaaggtttgtaaacacaccaatcagcaccctgtgtctagctcagggtttgtgaatgcaccaatcaacactctgtatctagctactctggtggggacttggagaacctttgtgtggacactctgtatctagctaatctggtggggacgtggagaacctttgtgtctagctcatggattgtaaatgcaccaatcagtgccctgtcaaaacagaccactgggctctaccaatcagcaggatgtgggtggggccagataagagaataaaagcaggctgcccgagccagcagtggcaacccgctcgggtccccttccacactgtggaagctttgttctttcgctctttgcaataaatcttgctgctgctcactgtttgggtctacactgcctttatgagctgtaacgctcaccgcgaaggtctgcagcttcactcttgaagccagcgagaccacgaacccaccgggaggaacgaacaactccagaggcgccgccttaagagctggaacgttcactgtgaaggtctgcagcttcactcctgagccagcgagaccacgaacccatcagaaggaagaaactccgaacacatccaaacatcagaacgaacaaactccacacacgcagcctttaagaactgtaacactcaccacgagggtccccggcttcattcttgaagtcagtgaaaccaagaacccaccaattccggacacagtatgtcagaaacaatatgagtcactaaatcaatatacttctcaacaatttccaacagcccttgcaattaacttggccatgtgactggttgtgactaaaataatgtggagataataatgtgttactccctaaggcagagtgcccttctatcattctctttcccttcctctatgtggcagaaagtaaaagattctgaaatgataaagtcaatcacaggaaggcacctggactcctggcccactgcttggaggagagcactcaggaccatgaacatctgactgtgacgtagcaataaagaaacccacgtttcatatgaaactgcttaaaattaatggcacaagtcatgtttttgatgttgcacatttgtctttatttgtggcttgttttgcttccacatcaatccactcaaggcctacattctgctataatgcaatttcaagttctttacaggccgagaaaaatgaatctgaattcctgacctccaaaagtgatcaagatatttttagttcaggctccaaaattttctcattttcataggttttcctcgattgatcattattcatgatttgcaaggaatcattcaatgttttctaaatctattactgcatcctgacacatatgacattttaactatgttccagatttttgaatgaagagtgtaaattttaaatgttttcaccacaaaaaataagtatgtgaagtggtggatttgttaattagccttatttaaccatttaatattgtacacgtacaccaaagcatcatgttgtaccccatgaatacacacaattattatttgtcaatttaaaatgaaataataaaaaataacaaaggcattagcctctgcattgcctttaccggtcatcctcacggtgactaacgcaaaaaacgttctatttcatccttacaaacatccctatctttgatgcctctttgtctagatctctatcccctcctgttttctctacgttatttatatgggtatcatcaccatcctggacaacatcaggacagatatccctcaccaagccaatgttcctctctatgttggctcaaatgtccttgaactttcctttcaccaccctttccacagtcaaaaggatattgtagtttaatgcctcagagttcagcttttaagcttctgacaaattattcttcctctttaggttctcctttatggaatcttctgtactgatggccatgtcctttaactactatgtagatatctgctactacctgtattatgcctctacctttattagcagagttatctgtactgttggcatgacaatcatttgttaatatgacttgcctttcctttttctgctattcttgatcaaatggctcctctttcttgctcctctcatttctcctgccttcacttggacgtgcttcacgtagtctgtgcttatgactggattaaaaattgatatggacttatcctaatgttgttcgtcataatatgggttttatggtccattattatttcctatgcattgatctggagaaggcttcaatccttttactctttgtggaaaatatctgtaaaccttctggttcactctgctatagcaatttcagtttaggctagtaagcatgaggatgcctccttctctgatttttcccacagtctgttggtcacagaataacctgagtgattactgatgaaagagtgagaatgttattgatagtcacaatgacaaaaaacaaacaactacagtcaaaatgtttctctttttattagtggattatatttcctgacctatatctggcaggactctttagagaggtagctgaagctgctgttatgaccactagagggaagaagatacctgtggagctaatggtccaagatggtggagccccaagcaaggaagttgttaaggagcccttttgattgaaggtgggtgcccccaccttacagggacaggacatctggatactcctcccagtttctccagtttccctttttcctaatatatctcctgataaaatgtctatactcacttccccatttctaataataaagcaaaggctagttagtaagacatcaccttgcattttgaaaatgccatagactttcaaaattatttcatacatcggtctttctttatttcaagagtccagaaatggcaacattacctttgattcaatgtaatggaaagagctctttcaagagacagagaaaagaataatttaatttctttccccacacctccttccctgtctcttaccctatcttccttccttctaccctccccatttctctctctcatttctcagaagtatattttgaaaggattcatagcagacagctaaggctggttttttctaagtgaagaagtgatattgagaaggtagggttgcatgagccctttcagttttttagtttatatacatctgtattgttagaatgttttataatataaataaaattatttctcagttatatactagctatgtaacctgtggatatttccttaagtattacaagctatacttaactcacttggaaaactcaaataaatacctgcttcatagttattaataaggattaagtgagataatgcccataagattcctattaataacagataaatacatacacacacacacacattgaaaggattcttactttgtgctaggaactataataagttcattgatgcattatatcattaagttctaatttcaacactagaaggcaggtattatctaaatttcatactggatacctccaaactcataaagataattaaattgccttttgtcatatatttattcaaaagggtaaactcaaactatggcttgtctaattttatatatcaccctactgaacatgaccctattgtgatattttataaaattattctcaagttattatgaggatgttgaaagacagagaggatggggtgctatgccccaaatcagcctcacaattaagctaagcagctaagagtcttgcagggtagtgtagggaccacagggttaagggggcagtagaattatactcccactttagtttcatttcaaacaatccatacacacacagccctgagcacttacaaattatactacgctctatactttttgtttaaatgtataaataagtggatgaaagaatagatagatagatagacagatagatgatagatagaataaatgcttgccttcatagctgtctccctaccttgttcaaaatgttcctgtccagaccaaagtaccttgccttcacttaagtaatcaattcctaggttatattctgatgtcaaaggaagtcaaaagatgtgaaaaacaatttctgacccacaactcatgctttgtagatgactagatcaaaaaatttcagccatatcttaacagtgagtgaacaggaaatctcctcttttccctacatctgagatcccagcttctaagaccttcaattctcactcttgatgcaacagaccttggaagcatacaggagagctgaacttggtcaacaaaggagaaaagtttgttggcctccaaaggcacagctcaaacttttcaagccttctctaatcttaaaggtaaacaagggtctcatttctttgagaacttcagggaaaatagacaaggacttgcctggtgcttttggtaggggagcttgcactttccccctttctggaggaaatatttatccccaggtagttccctttttgcaccagtggttctttgaagagacttccacctgggaacagttaaacagcaactacagggccttgaactgcacactttcagtccggtcctcacagttgaaaagacctaagcttgtgcctgatttaagcctttttggtcataaaacattgaattctaatctccctctcaaccctacagtcacccatttggtatattaaagatgtgttgtctactgtctagtatccctcaagtagtgtcaggaattagtcatttaaatagtctgcaagccaggagtggtggctcatgtctgtaattccagcacttgagaggtagaagtgggaggactgcttgagctcaagagtttgatattatcctggacaacatagcaagacctcgtctctacttaaaaaaaaaaaaaaaattagccaggcatgtgatgtacacctgtagtcccagctactcaggaggccgaaatgggaggatcccttgagctcaggaggtcaaggctgcagtgagacatgatcttgccactgcactccagcctggacagcagagtgaaaccttgcctcacgaaacagaatacaaaaacaaacaaacaaaaaactgctccgcaatgcgcttccttgatgctctaccacataggtctgggtactttgtacacattatctcattgctgttcataattgttagattaattttgtaatattgatattattcctagaaagctgaggcctcaagatgataacttttattttctggacttgtaatagctttctcttgtattcaccatgttgtaactttcttagagtagtaacaatataaagttattgtgagtttttgcaaacacagcaaacacaacgacccatatagacattgatgtgaaattgtctattgtcaatttatgggaaaacaagtatgtactttttctactaagccattgaaacaggaataacagaacaagattgaaagaatacattttccgaaattacttgagtattatacaaagacaagcacgtggacctgggaggagggttattgtccatgactggtgtgtggagacaaatgcaggtttataatagatgggatggcatctagcgcaatgactttgccatcacttttagagagctcttggggaccccagtacacaagaggggacgcagggtatatgtagacatctcattctttttcttagtgtgagaataagaatagccatgacctgagtttatagacaatgagcccttttctctctcccactcagcagctatgagatggcttgccctgcctctctactaggctgactcactccaaggcccagcaatgggcagggctctgtcagggctttgatagcactatctgcagagccagggccgagaaggggtggactccagagactctccctcccattcccgagcagggtttgcttatttatgcatttaaatgatatatttattttaaaagaaataacaggagactgcccagccctggctgtgacatggaaactatgtagaatattttgggttccatttttttttccttctttcagttagaggaaaaggggctcactgcacatacactagacagaaagtcaggagctttgaatccaagcctgatcatttccatgtcatactgagaaagtccccacccttctctgagcctcagtttctctttttataagtaggagtctggagtaaatgatttccaatggctctcatttcaatacaaaatttccgtttattaaatgcatgagcttctgttactccaagactgagaaggaaattgaacctgagactcattgactggcaagatgtccccagaggctctcattcagcaataaaattctcaccttcacccaggcccactgagtgtcagatttgcatgcactagttcacgtgtgtaaaaaggaggatgcttctttcctttgtattctcacatacctttaggaaagaacttagcacccttcccacacagccatcccaataactcatttcagtgactcaacccttgactttataaaagtcttgggcagtatagagcagagattaagagtacagatgctggagccagaccacctgagtgattagtgactcagtttctcttagtagttgtatgactcagtttcttcatctgtaaaatggagggttttttaattagtttgtttttgagaaagggtctcactctgtcacccaaatgggagtgtagtggcaaaatctcggctcactgcaacttgcacttcccaggctcaagcggtcctcccacctcaacatcctgagtagctggaaccacaggtacacaccaccatacctcgctaattttttgtatttttggtagagatggggtttcacatgttacacaggatggtctcagactccggagctcaagcaatctgcccacctcagccttccaaagtgctgggattataagcatgattacaggagttttaacaggctcataagattgttctgcagcccgagtgagttaatacatgcaaagagtttaaagcagtgacttataaatgctaactactctagaaatgtttgctagtattttttgtttaactgcaatcattcttgctgcaggtgaaaactagtgttctgtactttatgcccattcatctttaactgtaataataaaaataactgacatttattgaaggctatcagagactgtaattagtgctttgcataattaatcatatttaatactcttggattctttcaggtagatactattattatccccattttactacagttaaaaaaactacctctcaacttgctcaagcatacactctcacacacacaaacataaactactagcaaatagtagaattgagatttggtcctaattatgtctttgctcactatccaataaatatttattgacatgtacttcttggcagtctgtatgctggatgctggggatacaaagatgtttaaatttaagctccagtctctgcttccaaaggcctcccaggccaagttatccattcagaaagcattttttactctttgcattccactgtttttcctaagtgactaaaaaattacactttattcgtctgtgtcctgctctgggatgatagtctgactttcctaacctgagcctaacatccctgacatcaggaaagactacaccatgtggagaaggggtggtggttttgattgctgctgtcttcagttagatggttaactttgtgaagttgaaaactgtggctctctggttgactgttagagttctggcacttgtcactatgcctattatttaacaaatgcatgaatgcttcagaatatgggaatattatcttctggaatagggaatcaagttatattatgtaacccaggattagaagattcttctgtgtgtaagaatttcataaacattaagctgtctagcaaaagcaagggcttggaaaatctgtgagctcctcaccatatagaaagcttttaacccatcattgaataaatccctataggggatttctaccctgagcaaaaggctggtcttgattaattcccaaactcatatagctctgagaaagtctatgctgttaacgttttcttgtctgctaccccatcatatgcacaacaataaatgcaggcctaggcatgactgaaggctctctcataattcttggttgcatgaatcagattatcaacagaaatgttgagacaaactatggggaagcagggtatgaaagagctctgaatgaaatggaaaccgcaatgcttcctgcccattcagggctccagcatgtagaaatctggggctttgtgaagactggcttaaaatcagaagccccattggataagagtagggaagaacctagagcctacgctgagcaggtttccttcatgtgacagggagcctcctgccccgaacttccagggatcctctcttaagtgtttcctgctggaatctcctcacttctatctggaaatggtttctccacagtccagcccctggctagttgaaagagttacccatgcagaggccctcctagcatccagagactagtgcttagattcctactttcagcgttggacaacctggatccacttgcccagtgttcttccttagttcctaccttcgaccttgatcctcctttatcttcctgaaccctgctgagatgatctatgtggggagaatggcttctttgagaaacatcttcttcgttagtggcctgcccctcattcccactttaatatccagaatcactataagaagaatataataagaggaataactcttattataggtaagggaaaattaagaggcatacgtgatgggatgagtaagagaggagagggaaggattaatggacgataaaatctactactatttgttgagaccttttatagtctaatcaattttgctattgttttccatcctcacgctaactccataaaaaaacactattattatctttattttgccatgacaagactgagctcagaagagtcaagcatttgcctaaggtcggacatgtcagaggcagtgccagacctatgtgagactctgcagctactgctcatgggccctgtgctgcactgatgaggaggatcagatggatggggcaatgaagcaaaggaatcattctgtggataaaggagacagccatgaagaagtctatgactgtaaatttgggagcaggagtctctaaggacttggatttcaaggaattttgactcagcaaacacaagaccctcacggtgactttgcgagctggtgtgccagatgtgtctatcagaggttccagggagggtggggtggggtcagggctggccaccagctatcagggcccagatgggttataggctggcaggctcagataggtggttaggtcaggttggtggtgctgggtggagtccatgactcccaggagccaggagagatagaccatgagtagagggcagacatgggaaaggtgggggaggcacagcatagcagcatttttcattctactactacatgggactgctcccctatacccccagctaggggcaagtgccttgactcctatgttttcaggatcatcatctataaagtaagagtaataattgtgtctatctcatagggttattatgaggatcaaaggagatgcacactctctggaccagtggcctaacagttcaggacagagctatgggcttcctatgtatgggtcagtggtctcaatgtagcaggcaagttccagaagatagcatcaaccactgttagagatatactgccagtctcagagcctgatgttaatttagcaatgggctgggaccctcctccagtagaaccttctaaccagctgctgcagtcaaagtcgaatgcagctggttagactttttttaatgaaagcttagctttcattaaagattaagctcctaagcagggcacagatgaaattgtctaacagcaactttgccatctaaaaaaatctgacttcactggaaacatggaagcccaaggttctgaacatgagaaatttttaggaatctgcacaggagttgagagggaaacaagatggtgaagggactagaaaccacatgagagacacgaggaaatagtgtagatttaggctggaggtaaatgaaagagaagtgggaattaatacttactgaaatctttctatatgtcaggtgccattttatgatatttaataatctcattacatatggtaattctgtgagatatgtattattgaacatactataattaatactaatgataagtaacacctcttgagtacttagtatatgctagaatcaaatttaagtttatcatatgaggccgggcacggtggctcatatatgggattacatgcctgtaatcccagcactttgggaggccaaggcaattggatcacctgaggtcaggagttccagaccagcctggccaacatggtgaaaccccttctctactaaaaaatacaaaaaatcagccaggtgtggtggcacgcgtctataatcccagctactcaggaggctgaggcaggagaatcacttgaacccaggaggtggaggttgcagtgagctaagattgcaccactgcactccagcctaggcgacagagtgagactccatctcaaaaaaaaaaaaagaagtttattatatgaattaacttagttttactcacaccaatactcagaagtagattattacctcatttattgatgaggagcccaatgtacttgtagtgtagatcaacttattgaaagcacaagctaataagtagacaattagtaattagaagtcagatggtctgagctctcctactgtctacattacatgagctcttattaactggggactcgaaaatcaaagacatgaaataatttgtccaagcttacagaaccaccaagtagtaaggctaggatgtagacccagttctgctacctctgaagacagtgttttttccacagcaaaacacaaactcagatattgtggatgcgagaaattagaagtagatattcctgccctgtggcccttgcttcttacttttacttcttgtcgattggaagttgtggtccaagccacagttgcagaccatacttcctcaaccataattgcatttcttcaggaaagtttgagggagaaaaaggtaaagaaaaatttagaaacaacttcagaataaagagattttctcttgggttacagagattgtcatatgacaaattataagcagacacttgagaaaactgaaggcccatgcctgcccaaattaccctttgaccccttggtcaagctgcaactttggttaaagggagtgtttatgtgttatagtgttcatttactcttctggtctaacccattggctccgtcttcatcctgcagtgacctcagtgcctcagaaacatacatatgtttgtctagtttaagtttgtgtgaaattctaactagcgtcaagaactgagggccctaaactatgctaggaatagtgctgtggtgctgtgataggtacacaagaaatgagaagaaactgcagattctctgcatctccctttgccgggtctgacaacaaagtttccccaaattttaccaatgcaagccatttctccatatgctaactactttaaaatcatttggggcttcacattgtctttctcatctgtaaaaagaatggaagaactcattcctacagaactccctatgtcttccctgatgggctagagttcctctttctcaaaaattagccattattgtatttccttctaagccaaagctcagaggtcttgtattgcccagtgacatgcacactggtcaaaagtaggctaagtagaagggtactttcacaggaacagagagcaaaagaggtgggtgaatgagagggtaagtgagaaaagacaaatgagaagttacaacatgatggcttgttgtctaaatatctcctagggaattattgtgagaggtctgaatagtgttgtaaaataagctgaatctgctgccaacattaacagtcaagaaatacctccgaataactgtacctccaattattctttaaggtagcatgcaactgtaatagttgcatgtatatatttatcataatactgtaacagaaaacacttactgaatatatactgtgtccctagttctttacacaataaactaatctcatcctcataattctattagctaatacatattatcatcctatatttcagagacttcaagaagttaagcaacttgctcaagatcatctaagaagtaggtggtatttctgggctcatttggcccctcctaatctctcatggcaacatggctgcctaaagtgttgattgccttaattcatcagggatgggctcatactcactgcagaccttaactggcatcctcttttcttatgtgatctgcctgaccctagtagacttatgaaatttctgatgagaaaggagagaggagaaaggcagagctgactgtgatgagtgatgaaggtgccttctcatctgggtaccagtggggcctctaagactaagtcactctgtctcactgtgtcttagccagttccttacagcttgccctgatgggagatagagaatgggtatcctccaacaaaaaaataaattttcatttctcaaggtccaacttatgttttcttaatttttaaaaaaatcttgaccattctccactctctaaaataatccacagtgagagaaacattcttttcccccatcccataaatacctctattaaatatggaaaatctgggcatggtgtctcacacctgtaatcccagcactttgggaggctgaggtgggtggactgcttggagctcaggagttcaagaccatcttggacaacatggtgataccctgcctctacaaaaagtacaaaaattagcctggcatggtggtgtgcacctgtaatcccagctattagggtggctgaggcaggagaattgcttgaacccgggaggcggaggttgcagtgagctgagatcgtgccactgcactccagcctgggggacagagcacattataattaactgttattttttacttggactcttgtggggaataagatacatgttttattcttatttatgattcaagcactgaaaatagtgtttagcatccagcaggtgcttcaaaaccatttgctgaatgattactatactttttacaagctcagctccctctatcccttccagcatcctcatctctgattaaataagcttcagtttttccttagttcctgttacatttctgtgtgtctccattagtgacctcccatagtccaagcatgagcagttctggccaggcccctgtcggggtcagtgccccacccccgccttctggttctgtgtaaccttctaagcaaaccttctggctcaagcacagcaatgctgagtcatgatgagtcatgctgaggcttagggtgtgtgcccagatgttctcagcctagagtgatgactcctatctgggtccccagcaggatgcttacagggcagatggcaaaaaaaaggagaagctgaccacctgactaaaactccacctcaaacggcatcataaagaaaatggatgcctgagacagaatgtgacatattctagaatatattatttcctgaatatatatatatatatacacatatacgtatatatatatatatatatatatttgttgttatcaattgccatagaatgattagttattgtgaatcaaatatttatcttgcaggtggcctctatacctagaagcggcagaatcaggctttattaatacatgtgtatagatttttaggatctatacacatgtattaatatgaaacaaggatatggaagaggaaggcatgaaaacaggaaaagaaaacaaaccttgtttgccattttaaggcacccctggacagctaggtggcaaaaggcctgtgctgttagaggacacatgctcacatacggggtcagatctgacttggggtgctactgggaagctctcatcttaaggatacatctcaggccagtcttggtgcattaggaagatgtaggcaactctgatcctgagaggaaagaaacattcctccaggagagctaaaagggttcacctgtgtgggtaactgtgaaggactacaagaggatgaaaaacaatgacagacagacataatgcttgtgggagaaaaaacaggaggtcaaggggatagagaaggcttccagaagaatggctttgaagctggcttctgtaggagttcacagtggcaaagatgtttcagaaatgtgacatgacttaaggaactatacaaaaaggaacaaatttaaggagaggcagataaattagttcaacagacatgcaaggaattttcagatgaatgttatgtctccactgagcttcttgaggttagcagctgtgagggttttgcaggcccaggacccattacaggacctcacgtatacttgacactgttttttgtattcatttgtgaatgaatgacctcttgtcagtctactcggtttcgctgtgaatgaatgatgtcttgtcagcctacttggtttcgctaagagcacagagagaagatttagtgatgctatgtaaaaacttcctttttggttcaagtgtatgtttgtgatagaaatgaagacaggctacatgatgcatatctaacataaacacaaacattaagaaaggaaatcaacctgaagagtatttatacagataacaaaatacagagagtgagttaaatgtgtaataactgtggcacaggctggaatatgagccatttaaatcacaaattaattagaaaaaaaacagtggggaaaaaattccatggatgggtctagaaagactagcattgttttaggttgagtggcagtgtttaaagggtgatatcagactaaacttgaaatatgtggctaaataactagaatactctttattttttcgtatcatgaatagcagatatagcttgatggccccatgcttggtttaacatccttgctgttcctgacatgaaatccttaatttttgacaaaggggctattcattttcattttatattgggcctagaaattatgtagatggtcctgaggaaaagtttatagcttgtctatttctctctctaacatagttgtcagcacaatgcctaggctataggaagtactcaaagcttgttaaattgaattctatccttcttattcaattctacacatggaggaaaaactcatcagggatggaggcacgcctctaaggaaggcaggtgtggctctgcagtgtgattgggtacttgcaggacgaagggtggggtgggagtggctaaccttccattcctagtgcagaggtcacagcctaaacatcaaattccttgaggtgcggtggctcactcctgtaatcacagcagtttgggacgccaaggtgggcagatcacttgaggtcaggagttggacaccagcccagccaacatagtgaaacctggtctctgcttaaaaatataaaaattagctggacgtggtgacgggagcctgtaatccaactacttgggaggctgaggcaggagaatcgcttgaaccggggaggtggagtttgcactgagcagagatcatgccattgcactccagcctccagagcgagactctgtctaaagaaaaacgaaaacaaacaaacaaacaaacaaacaaaacccatcaaattccctgaccgaacagaattctgtctgattgttctctgacttatctaccattttccctccttaaagaaactgtgaacttccttcagctagaggggcctggctcagaagcctctggtcagcatccaagaaatacttgatgtcactttggctaaaggtatgatgtgtagacaagctccagagatggtttctcatttccatatccacccacccagctttccaattttaaagccaattctgaggtagagactgtgatgaacaaacaccttgacaaaattcaacccaaagactcactttgcctagcttcaaaatccttactctgacatatactcacagccagaaattagcatgcactagagtgtgcatgagtgcaacacacacacacaccaattccatattctctgtcagaaaatcctgttggtttttcgtgaaaggatgttttcagaggctgaccccttgccttcacctccaatgctaccactctggtctaagtcactgtcaccaccacctaaattatagctgttgactcataacaatcttcctgcttctaccactgccccactacaatttcttcccaatatactatccaaattagtcttttcaaaatgtaagtcatatatggtcacctctttgttcaaagtcttctgatagtttcctatatcatttataataaaaccaaatccttacaattctctacaatagttgttcatgcatatattatgtttattacagatacatatatatagctctcatataaataaatatatatatttatgtgtatgtgtgtagagtgttttttcttacaactctatgatgtaggtattattagtgtcccaaattttataatttaggacttctatgatctcatcttttattctccccttcaccgaatctcatcctacattggccttattgatattccttgaaaattctaagcatcttacatctttagggtatttacatttgccattccctatgccctaaatatttaatcatagtttcatataaatgggttcctcatcatctatgggtactctctcaggtgttaactttatagtgaggactttcctgccatactacttaaagtagcgataccctttcaccctgtcctaatcacactctggccttcatttcagttttttttttttctccatagcacctaatctcattggtatataacatgtttcatttgcttatttaatgtcaagctctttccactatcaagtccatgaaaacaggaactttattcctctattctgtttttgtgctgtattcttagcaattttacaattttgaatgaatgaatgagcagtcaaacacatatacaactataattaaaaggatgtatgctgacacatccactgctatgcacacacaaagaaatcagtggagtagagctggaagtgctaagcctgcatagagctagttagccctccgcaggcagagccttgatgggattactgagttctagaattggactcatttgttttgtaggctgagatttgctcttgaaaacttgttctgaccaaaataaaaggctcaaaagatgaatatcgaaaccagggtgttttttacactggaatttataactagagcactcatgtttatgtaagcaattaattgtttcatcagtcaggtaaaagtaaagaaaaactgtgccaaggcaggtagcctaatgcaatatgccactaaagtaaacattatttcataggtgtcagatatggcttattcatccatcttcatgggaaggatggccttggcctggacatcagtgttatgtgaggttcaaaacacctctaggctataaggcaacagagctccttttttttttttctgtgctttcctggctgtccaaatctctaatgataagcatacttctattcaatgagaatattctgtaagattatagttaagaattgtgggagccattccgtctcttatagttaaatttgagcttcttttatgatcactgtttttttaatatgctttaagttctggggtacatgtgccatggtggtttgctgcacccatcaacccgtcatctacattaggtatttctcctaatgctatccttcccctagccccccacccccaacaggccccagtgtgtgatgttcccctccctgtgtccatggatcactggtttttttttgttttttttttttttttaaagtctcagttaaatttttggaatgtaatttattttcctggtatcctaggacttgcaagttatctggtcactttagccctcacgttttgatgataatcacatatttgtaaacacaacacacacacacacacacacacacatatatatatatataaaacatatatatacataaacacacataacatatttatcgggcatttctgagcaactaatcatgcaggactctcaaacactaacctatagccttttctatgtatctacttgtgtagaaaccaagcgtggggactgagaaggcaatagcaggagcattctgactctcactgcctttagctaggcccctccctcatcacagctcagcatagtcctgagctcttatctatatccacacacagtttctgacgctgcccagctatcaccatcccaagtctaaagaaaaaaataatgggtttgcccatctctgttgattagaaaacaaaacaaaataaaataagcccctaagctcccagaaaacatgactaaaccagcaagaagaagaaaatacaataggtatatgaggagactggtgacactagtgtctgaatgaggcttgagtacagaaaagaggctctagcagcatagtggtttagaggagatgtttctttccttcacagatgccttagcctcaataagcttgcggttgtggaagtttactttcagaacaaactcctgtggggctagaattattgatggctaaaagaagcccgggggagggaaaaatcattcagcatcctcacccttagtgacacaaaacagagggggcctggttttccatatttcctcatgatggatgatctcgttaatgaaggtggtctgacgagatcattgcttcttccatttaagccttgctcacttgccaatcctcagttttaaccttctccagagaaatacacattttttattcaggaaacatactatgttatagtttcaatactaaataatcaaagtactgaagatagcatgcataggcaagaaaaagtccttagctttatgttgctgttgtttcagaatttaaaaaagatcaccaagtcaaggacttctcagttctagcactagaggtggaatcttagcatataatcagaggtttttcaaaatttctagacataagattcaaagccctgcacttaaaatagtctcatttgaattaactctttatataaattgaaagcacattctgaactacttcagagtattgttttatttctatgttcttagttcataaatacattaggcaatgcaatttaattaaaaaaacccaagaatttcttagaattttaatcatgaaaataaatgaaggcatctttacttactcaaggtcccaaaaggtcaaagaaaccaggaaagtaaagctatatttcagcggaaaatgggatatttatgagttttctaagttgacagactcaagttttaaccttcagtgcccatcatgtaggaaagtgtggcataactggctgattctggctttctactcctttttcccattaaagatccctcctgcttaattaacattcacaagtaactctggttgtactttaggcacagtggctcccgaggtcagtcacacaataggatgtctgtgctccaagttgccagagagagagattactcttgagaatgagcctcagccctggctcaaactcacctgcaaacttcgtgagagatgaggcagaggtacactacgaaagcaacagttagaagctaaatgatgagaacacatggactcatagagggaaacaacgcatactggggcctatcagagggtggagggtgagagaaggagaggatcaggaaaaatcactaatggatgctaagcgtaatacctgagtgatgagatcatctatacaacaaacccccttgacattcatttatctatgtaacaaacctgcacatcctgtacatgtacccctgaacttaaaataaaagttgaaaacaagaaagcaacagtttgaacacttgttatggtctattctctcattctttacaattacactagaaaatagccacaggcttcctgcaaggcagccacagaatttatgacttgtgatatccaagtcattcctggataatgcaaaatctaacacaaaatctagtagaatcatttgcttacatctatttttgttctgagaatatagatttagatacataatggaagcagaataatttaaaatctggctaatttagaatcctaagcagctcttttcctatcagtggtttacaagccttgtttatatttttcctattttaaaaataaaaataaagtaagttatttgtggtaaagaatattcattaaagtatttatttcttagataataccatgaaaaacattcagtgaagtgaagggcctactttacttaacaagaatctaatttatataatttttcatactaatagcatctaagaacagtacaatatttgactcttcaggttaaacatatgtcataaattagccagaaagatttaagaaaatattggatgtttccttgtttaaattaggcatcttacagtttttagaatcctgcatagaacttaagaaattacaaatgctaaagcaaacccaaacaggcaggaattaatcttcatcgaatttgggtgtttctttctaaaagtcctttatacttaaatgtcttaagacatacatagattttattttactaattttaattatatagacaataaatgaatattcttactgattactttttctgactgtctaatctttctgatctatcctggatggccataacacttatctctctgaactttgggcttttaatataggaaagaaaagcaataatccatttttcatggtatctcatatgataaacaaataaaatgcttaaaaatgagcaggtgaagcaatttatcttgaaccaacaagcatcgaagcaataatgagactgcccgcagcctacctgacttctgagtcaggatttataagccttgttactgagacacaaacctgggcctttcaatgctataacctttcttgaagctcctccctaccacctttagccataaggaaacatggaatgggtcagatccctggatgcaagccaggtctggaaccataggcagtaaggagagaagaaaatgtgggctctgcaactggctccgagggagcaggagaggatcaaccccatactctgaatctaagagaagactggtgtccatactctgaatgggaagaatgatgggattacccatagggcttgttttagggagaaacctgttctccaaactcttggccttgagatacctggtccttattccttggactttggcaatgtctgaccctcacattcaagttctgaggaagggccactgccttcatactgtggatctgtagcaaattccccctgaaaacccagagctgtatcttaattggttaaaaaaaattatattatctcaacgactgttcttctctgagtagccaagctcagcttggttcaagctacaagcagctgagctgctttttgtctagtcattgttcttttatttcagtggatcaaatacgttctttccaaacctaggatcttgtcttcctaggctatatattttgtcccaggaagtcttaatctggggtccacagaacactagggggctggtgaagtttatagaaaaaaaatctgtatttttacttacatgtaactgaaatttagcattttcttctactttgaatgcaaaggacaaactagaatgacatcatcagtacctattgcatagttataaagagaaaccacagatattttcatactacaccataggtattgcagatctttttgtttttgtttttgtttgagatggagtttcgctcttattgcccaggctggagtgcagtggcatgatttcggctcactgcaacctccccttcctgcattcaagcaattctcctgccttggcctcctgagtagctggggattacaggcacctgccaccatgccagtctaatttttgtatttttagtagagatggggtttcgccatgttggccaggctggtcttgaactcctgacctcagatgatctgcccgccttggcctcctgaagtgctgggattataggtgtgagccaccacgcctggcccattgcagatatttttaattcacatttatctgcatcactacttggatcttaaggtagctgtagacccaatcctagatctaatgctttcataaagaagcaaatataataaatactataccacaaatgtaatgtttgatgtctgataatgatatttcagtgtaattaaacttagcactcctatgtatattatttgatgcaataaaaacatatttttttagcacttacagtctgccaaactggcctgtgacacaaaaaaagtttaggaattcctggttttgtctgtgttagccaatggttagaatatatgctcagaaagataccattggttaatagctaaaagaaaatggagtagaaattcagtggcctggaataataacaatttgggcagtcattaagtcaggtgaagacttctggaatcatgggagaaaagcaagggagacattcttacttgccacaagtgtttttttttttttttttttttatcacaaacataagaaaatataataaataacaaagtcaggttatagaagagagaaacgctcttagtaaacttggaatatggaatccccaaaggcacttgacttgggagacaggagccatactgctaagtgaaaaagacgaagaacctctagggcctgaacatacaggaaattgtaggaacagaaattcctagatctggtggggcaaggggagccataggagaaagaaatggtagaaatggatggagacggaggcagaggtgggcagatcatgaggtcaagagatcgagaccatcctggcaaacatggtgaaatcccgtctctactaaaaataaaaaaattagctgggcatggtggcatgcgcctgtagtcccagctgctcgggaggctgaggcaggagaatcgtttgaacccaggaggcgaaggttgcagtgagctgagatagtgccattgcactccagtctggcaacagagtgagactccgtctcaaaaaaaaaaaaaaaagaaagaaagaaaagaaaaagaaaaaagaaaaaataaatggatgtagaacaagccagaaggaggaactgggctggggcaatgagattatggtgatgtaagggacttttatagaattaacaatgctggaatttgtggaactctgcttctattattcccccaatcattacttctgtcacattgatagttaaataatttctgtgaatttattccttgattctaaaatatgaggataatgacaatggtattataagggcagattaagtgatatagcatgagcaatattcttcaggcacatggatcgaattgaatacactgtaaatcccaacttccagtttcagctctaccaagtaaagagctagcaagtcatcaaaatggggacatacagaaaaaaaaaaggacactagaggaataatataccctgactcctagcctgattaatatatcgattcacttttttctctgtttgatgacaaattctggctttaaataattttaggattttaggcttctcagctcccttcccagtgagaagtataagcaggacagacaggcaagcaagaagagagccccaggcaatactcacaaagtagccaatgtcccctgtggtcatagagaaatgaaaagagagaggattctctggaagcactggatgtaatcttttctgtctgtcctctctagggaatcaccccaaggtactgtactttgggattaaggctttagtcccactgtggactacttgctattctgttcagtttctagaaggaactatgtacggtttttgtctccctagagaaactaaggtacagaagttttgtttacaatgcactccttaagagagctagaactgggtgagattctgttttaacagctttattttcttttccttggccctgtttttgtcactgtcaccacctttaaggcaaatgttaaatgcgctttggctgaaactttttttcctattttgagatttgctcctttatatgaggctttcttggaaaaggagaatgggagagatggatatcattttggaagatgatgaagagggtaaaaaaggggacaaatggaaatttgtgttgcagatagatgaggagccaacaaaaaagagcctcaggatccagcacacattatcacaaacttagtgtccatccatcactgctgaccctctccggacctgactccacccctgagggacacaggtcagccttgaccaatgacttttaagtaccatggagaacagggggccagaacttcggcagtaaagaataaaaggccagacagagaggcagcagcacatatctgcttccgacacagctgcaatcactagcaagctctcaggcctggcatcatggtgcattttactgctgaggagaaggctgccgtcactagcctgtggagcaagatgaatgtggaagaggctggaggtgaagccttgggcaggtaagcattggttctcaatgcatgggaatgaagggtgaatattaccctagcaagttgattgggaaagtcctcaagattttttgcatctctaattttgtatctgatatggtgtcatttcatagactcctcgttgtttacccctggacccagagattttttgacagctttggaaacctgtcgtctccctctgccatcctgggcaaccccaaggtcaaggcccatggcaagaaggtgctgacttcctttggagatgctattaaaaacatggacaacctcaagcccgcctttgctaagctgagtgagctgcactgtgacaagctgcatgtggatcctgagaacttcaaggtgagttcaggtgctggtgatgtgattttttggctttatattttgacattaattgaagctcataatcttattggaaagaccaacaaagatctcagaaatcatgggtcgagcttgatgttagaacagcagacttctagtgagcataaccaaaacttacatgattcagaactagtgacagtaaaggactactaacagcctgaattggcttaacttttcaggaaatcttgccagaacttgatgtgtttatcccagagaattgtattatagaattgtagacttgtgaaagaagaatgaaatttggcttttggtagatgaaagtccatttcaaggaaatagaaatgccttattttatgtgggtcatgataattgaggtttagaaagagatttttgcaaaaaaaataaaagatttgctcaaagaaaaataagacacattttctaaaatatgttaaatttcccatcagtattgtgaccaagtgaaggcttgtttccgaatttgttggggattttaaactcccgctgagaactcttgcagcactcacattctacatttacaaaaattagacaattgcttaaagaaaaacagggagagagggaacccaataatactggtaaaatggggaagggggtgagggtgtaggtaggtagaatgttgaatgtagggctcatagaataaaattgaacctaagctcatctgaattttttgggtgggcacaaaccttggaacagtttgaggtcagggttgtctaggaatgtaggtataaagccgtttttgtttgtttgtttgttttttcatcaagttgttttcggaaacttctactcaacatgcctgtgtgttattttgtcttttgcctaacagctcctgggtaacgtgatggtgattattctggctactcactttggcaaggagttcacccctgaagtgcaggctgcctggcagaagctggtgtctgctgtcgccattgccctggcccataagtaccactgagttctcttccagtttgcaggtgttcctgtgaccctgacaccctccttctgcacatggggactgggcttggccttgagagaaagccttctgtttaataaagtacattttcttcagtaatcaaaaattgcaattttatcttctccatcttttactcttgtgttaaaaggaaaaagtgttcatgggctgagggatggagagaaacataggaagaaccaagagcttccttaagaaatgtatgggggcttgtaaaattaatgtggatgttatgggagaattccaggattccaaggaggatgatatgatggagaaaaatctttatcggggtgggaaaatggttaattaagtggacagagactcctaggcagtttttactgcaccggggaaagaaggagctgttagtggtacctgagaaagcagatttgtggtacatgtcacttttcattaaaaacaaaaacaaaacaaaacaaaacttcatagatatccaagatataggctagaattactattttaatttactcttatttacattttgaagtagctagcttgtcacatgttttatgaaattgatttggagataagatgagtgtgtatcaacaatagcctgctctttccatgaaggattccattatttcatgggttagctgaagctaagacacatgatatcattgtgcattatcttctgatagaatgtaacatgcactaaaataaagttagagttaggacctgagtgggaaagtttttggagagtgtgatgaagactttccgtgggagatagaatactaataaaggcttaaattctaaaaccagcaagctagggcttcgtgacttgcatgaaactggctctctggaagtagaagggagagtaagacatacgtagaggactaggaaagaccagatagtacagggcctggctacaaaaatacaagcttttactatgctattgcaatactaaacgataagcattaggatgttaagtgactcaggaaataagattttgggaaaaagtaatctgcttatgtgcacaaaatggattcaagtttgcagataaaataaaatatggatgatgattcaaggggacagatacaatggttcaaacccaagaggagcagtgagtctgtggaatttgaaggatggacaaaggtggggtgagaaagacatagtattcgactgactgtgggagatgagaaggaagaaggaggtgataaatgactgaaagctcccagactggtgaagataacaggaggaaaccatgcactgacctggtgactctcatgtgtgaagggtagagggatattaacagatttactttttaggaagtgctagattggtcagggagttttgaccttcaggtcttgtgtctttcatatcaaggaacctttgcattttccaagttagagtgccatattttggcaaatataactttattagtaattttatagtgctctcacattgatcagactttttcctgtgaattacttttgaatttggctgtatatatccagaatatgggagagagacaaataattattgtagttgcaggctatcaacaatactggtctctctgagccttataacctttcaatatgcccataaacagagtaaacagggattattcatggcactaaatattttcacctagtcagtcaacaaatgggagcaatgtgcattttttgatacatatttttatatatttatggggtacatgtgatacttacatgcctagaacatgtgatgattaagtctagatatttaggatatccattgctttgagcatttatcatttctatgtattgagaaaatttcaaatcctcatttctagccattttgaaatatataataaatagtaattaactatagtcaccctactcaaatatcaaacattatggcttaatccttctatccaactgtgtttgtacctattaaccaacatctcttaaatcccctcccatacacactcacactttttccagcctctgataactatcattctactctctaccaccatgagacccacttttttagctcccacagatgaataaaaacatgtgatatttgactttctgtatctggcttattttattatctatctctttggcataccaagagtttgtttttgttctgcttcagggctttcaattaacataatgacctctggttccatccatgttgctacaaatgacaagatttcattctttttcatggcaaaatagtactgtgcaaaaatacaattttttaatccgttcatctgttgatagacacttaggttgatcccaaaccttaactattgtgaatagtgcttcaataaacatgagtgtaatgtgtccattggatatactgatttcctttcttttggataaataaccactagtgagattgctggattgtatgatagttctgtttttagtttactgagaaatcttcatactgttttccataatggttgtactattttacattcccaccaacagtgtgtaagaaagagttcccttttctccatatcctcacaaggatctgttattttttgtcttttttgttaatagccgttttaactagagtaagtagatatctcattgtagttttgatttgcatttccctgatcattagtgatgttgagaattttttcatatgtttgttggtcatttgtatatctttttctgagaattgtctgttcatgtccttagcctactttttattgggattgtttgttattttcttgataatctatttgtgttcattttagagcctggatattattcttttgtcagatgtatagattgtgaagattttctcccactctgtgggttgtctgtttattctgcagactcttccttttgccatgcaaaagctctttagtttaatttagtcccagatattttctttgtttttatgtatttgcatttgtgttcttggtcatgaaatcctttcctaagccaatgtgtagaagggtttttccgatgttattttctagaattgttacagtttcagggcttagatttaagtccttgatccatcttgagttgatttttgtataaggtgagagatgaagatccagtttcattctcctacatgtagcttgccagctatccccgcaccatttgttgaatagggtgccctttccccactttatgtttttgtttgctttgtcaaagatcagttggatgtaagtatttgagtttatttctgggttctctattctgttccattggtcgatgtgcctatttgtacaccagcatcatgctgttttggtgactatggccttattgtatagtttgaaatgaggtaatgtaatgccttcagatttgttcttttttttagacttgcttgtttattgggctcttttttggttccataagaattttaggattgttttttctagttctgtgaagactaatggtggtattttgatgggaattgcaatgaatttgtaggttgcttctggcattatggccattttcacaatattgattctacccatctatgagaatggcatgtgtttccatttgtttgtgtcttatatgattactttcagccgtgttttgtagttttccttgtagatgtctttcacctccttggttaggtatatattcctaagtttttgttttgttttgttttgttttttgcagctattgtaaaaggggttgagttcttgattttattctcagcttggtcattgctggtatgtaagaaagcaactcattggtgtacgttaattttgtatccagaaactttgctgaattattttatcagttctagggggttttggaggagtctttagagttttctacatacacaatcatatcatcagcaaacagtgacagtttgactttctctttaacaatttggatgtgctttacttgtttctcttgtctgattgctcttgctaggacttccagtaatatgttaaagagaagtggtgagagtgggtatccttgtctcattccagttttcagacagaatgcttttaactttttcccattcaatataatgttggctgtgtgtttaccatagctggcttttattacattgaggtatgtcctttgtaaaccgattttgctgagttttagtcataaagtgatgttgaattttgttgaatgcagtttctgtggctattgagataatcacatgatttttgtttccaattctctttatgttgtgtatcacacttattgacttgcgtatgttaaaccatccgtgcatccctcgcatgaaacccacttgatcatgggttttgatatgctgtcggatgctattagctagtattttgtcaaggatgttggcatctatgttcatcagggatattgatctgtagtgttttttttttttggttatgttctttcccagttttggtattaaggtgatactggcttcatagaatgatttagggaggattctctctttctctatcttgtagaatactgtcaataggattggtatcaattcttctttgaatgtctggtagaattcagctgtgaatctatctggtcctggacttttttgttgttggtaaatttttattatcatttcagtcttgctgcttattactggtctgttcagggtatctaattcttcctgacttaagctagagccctgtatctttccaggaattcgaacgtctcctttaggttttctagtttatgcatgtaaaggtgttcatagtagccttgaataatcttttgtatttctgtggtatcagtaatagtatctcctgttttgtttctaattgagtttatttgcacttctctcctcttttcttggttaatcttgctaatggtctatcagttttatttatcttttcaaagaaccagctttttatttcatttagcttttgtatttttttgcagttgttttaatttcatttagttctcctcttatcttagttattccctttcttttgctgggttttggttctgtttgtttttgtttctctagtttcttgtggtgtgaccttatattgtctgtctgtcctctttcagactctttgacatcgacatttagggctgtgaactttccttttagcaccatctttgctgtatcctagaggttttgataggttgtgtcactattgtcggtcagttcaagtaattttgttgttcttattatactttaagttctgggatacatgtgcagaatgtgcaggtttgttacataggtatagatgtgccatggtggtttgctgcacccatcaacctgtcatctacattaggtatttcttttaatgttatccctctcctaaccccctcaccccccgacaggccctggtgtgtgatgttcccctccctgtgtccatgtgttctcattgttcaactcccacttatgagtgagaacgtgtggtgtttggtttctctgttcctgtgttagtttgctcagaatgatggtttccaccttcatccatgtccctgcaaagacatgaactcatcatttttatggctgcatagtattccatggtgtatatgtgccacattttctttatccattatatcgctgatggccatttgggttggttccaagtctttgctattgtgaatagtgccacaataaacatacgtgtgcacgtgtctttatagtagaatgatttctaattctttgggtatatacccagtaatgggattgctgggtcaaacagtatttctggttctagatccttgaggaatcgccacactgtcttccacaatggttgaactaatttacacacccatcaacagtgtaaaatttttcctattcttccacatcctctccagcaccttttgtttcctgactttttaataattgccattctaactggcatgagatggtatctcattgtggttttgatttgcatttctctaatgaccagtgatgatgagcttcttttcatgtgtttcttggccacataaatgacttctttagagaagcatctgttcatatcctttgtccactttttgatggggtcgttaggttttttcttgtaaatttgttgaagttctttgtagattttggatgttagccctttgtcagatggatagattgcaaaaattttctcccattctgtaggttgcctgttcactctgatgatagtcttttgctgtgcagaagctctttagtttaattagatcccatatgtcaattttggcctttgttgtcattgcttttgatgttttagtcgtgaatttttgcccatgcctatgtcctgaatggtattgcctaggttatcttctaggatttttatggttttaggttgcacatttaagtctttaatccaccttgagttaatttttgtataaggtgtaaggaaggggtacagtttcagttttatgcatattgctagccagtttttccagcaccatttattaaatagggaattctttctccattgcttttgtgatgtttgtcaaagatcagatggtcgtagatgtgtggcattatttctgaggcttctgttctgttccactggtctatatatctgttttggtaccagtaccatgctgtttttgttactgtagccttgtagtatagtttgaagtcaggtagcatcatgcctccagctttgttctttttgtttaggattgtcttggctatatgggctcttttttgattccatatgacatttaaagtagttttttctaattctttgaaaaaagtcagtggtagcttgatggggatagcattgaatctataaattactttgggcagtatggccattttaaagatattgattctttctatctatgagcatggaatgtttttccatttgtttgtgtcctctcttatttccttgagcagtgagtggtttgtagctctccttgaagaggttcttcacatcccttagaagttgtatttctaggtattttattttattctctttgcagcaattgtgaatgggagttcacccatgatttggctctctgcttgtctattattggtgtataggaacgcttgtgatttctgcacactgattttgtatcttgagactttgctgaagctgtttatcagcttaagattttgggctgagatgacagggtcttctaaatatacaatcatgtcatctgcaaacagagacaatttgacttcctctcttcctatttgaatatgctttatttctttctcttgcctgattgtcctggcgagaacttccaatactatgttgagtaagagtggcgagagggcatccttgtcttgtgccggttttcaaagcaaatgatttttaaatttccatcttgatttcattgttgacccaatgatcattcaggagcaggttatttaatttccctgtatttgcatggttttgaaggttccttttgtagttgatttccaattttattctactgtggtctgagagagtgcttgatataatttcaatttttaaaaatttattgaggcttgttttgtggcatatcatatggcctatcttggagaaagttccatgtgctgatgaatagaatgtgtattctgcagttgttgggtagaatgtcctgtaaatatctgttaagtccatttgttctttaaatccattgtttctttgtagactgtcttgatgacctgcctagtgcagtcagtggagtattgaagtcccccactattattatgttgctgtctagtctagtagtaattgttttataaatttgggatctccagtattagatgcatatatattaagaattgtaatattctcccattggacaagggcttttatcattatatgatgtccctctttgtcttttttaactgctgtttctttaaagtttgttttgtctgacataagaatagctgctttggctcgcttttggtgtccatttgtgtggaatgtcattttccacccctttaccttaagtttatgtgagtccttatgtgttaggtgagtctcctgaaggcggcagataactggttggtgaattcttattcattctgcaattctgtatcttttaagtggagcatttagtccatttacattcaacatcagtattgaggtgtgaggtactattccattcttcgtggtatttgttgcctgtgtatctttttatctgtatttttgttgtatatgtcctatgggatttatgctttaaagaggttctgttttgatgtgcttccagggtttatttcaagatttagagctccttttatcagttcttgtagtgttggcttggtagtgccgaattctctcagcatttgtttttctgaaaaacactgtgtattttcttcatttgtgaagcttagtttcactggatataaaattcttggctgataattgttttgtttaagaaggctgaagatagggccatattcacttctagcttttacggtttctgctgagaaatctgctgttaatctgataggttttctttcataggttacctggtagtttcacctcacagctcttaagattctctttgtctttagataactttggatactctgatgacaatgtacctaggcaatgatatttttgcaatgaatttcccaggtgtttattgagcttcttgtatttggatatctaggtctctagcaaggtgggggaagttttccttgattatttccctggataagttttccaaacttttagatttctcttctttctcaggaatgctgattattcttaggtttgattgtttaacataatcccagatttcttggaggctttgttcatattttcttattcttttttctttgtctttgttggattgggttaattcaaaaactttgtcttcaagctctgaatttcttctgcttggattctattgctgagactttctagagcattttgcatttctataagtgcatccattcatccattgtttcctgaagttttgaatgttttttatttatgctatctctttaactgaagatttctcccctcatttcttgtatcatatttttggtttttttaaaattggacttcaccttcctcggatgcctccttgattagcttaataactgaccttctgaattatttttcaggtaaatcagggatttcttcttggtttggatgcattgctggtgagctagtatgattttttggggggtgttaaagaaccttgtttttcatattaccagagttagttttctggttccttctcacttgggtaggctctgtcagagggaaagtctaggcctcaaggctgagacttttgtcccatgaggtgttcccttgatgtagcacagtcccccttttcctaggcgtggggcttcctgagagccgaactgtagtgattgttatctctcttctggatctagccacccatcaggtctaccagactccaggctggtactggggtttgtctgcacagagtcttgtgacgtgaaccatctgtgggtctctcagccatagatacaaccacctgctccaatggaggtggcagaggatgaaatggactctgtgagggtccttacttttggttgttcaatgcactatttttgtgctggttggcctcctgccaggaggtggcactttctagaaagcatcagcagaggcagtcaggtggtggtggctgggggggctggggcaccctagaactcccaagaatatatgccctttgtcttcagctaccagggtgagtaaggaaggaccatcaggtgggggcaggactagtcgtgtctgagctcagagtctccttgggcaggtctttctgtggctactgtgggaggatgggggtgtagtttccaggtcaatggatttatgttcctaggacaattatggctgcctctgctgtgtcatgcaggtcatcaggaaagtgggggaaagcaagcagtcacgtgacttgcccagctcccatgcaactcaaaaggttggtctcacttccagcgtgcaccctcccccgcaacagcaccgaatctgtttccatgcagtcagtgagcaaggctgagaacttgccccaggctaccagctgcgaaaccaagtagggctgtcctacttccctgccagtggagtctgcacaccaaattcatgtccccccaccaacccccccactgcccagcccctagatctggccaggtggagattttctttttcctgtcatcttttcccagttcctctggcagccctcccaaatgacccctgtgaggcaaggcagaaatggcttcctaggggacccagagagcccacagggcttttcccgctgcttcctctacccctgtattttgcttggccctctaaattgactcagctccaggtaaggtcagaatcttctcctgtggtctagatcttcaggttccccagtgaggatgtgtgtttgggggtagacggtcccccttttccacttccacagtttgggcactcacaatatttggggtgtttcccgggtcctgcaggagcaatctgcttctttcagagggtgtgtgcgttctctcagctttcttgatttatttctgcaggtggttctgcaaaaaaaattcctgatgggagacttcacatgctgctctgtgcatccgagtgggagctgcaatgtacttctgctgcctcccatctgccatcaccctctaatttgtcggtaatatgcatttttaatcaatctttttttctctctctctctttttcttctcccccaaaactatactgccctttgatatcaaggaatcaaggacgtgatgttgaggggtgggcagtggatacactctttaccccttagggagctatatctagatttagatattgccaattcaagataacttaattgaaagcaaattcataatgaatacacacacacacacacacatctgcatgacaagatttttaatagttgaaagaataactaataattgtccacaggcaataagggctttttaagcaaaacagttgtgataaacaggtcattcttagaatagtaatccagccaatagtacaggttgcttagagattatgtcattaccagagttaaaattctataatggcttctcactccctaccactgaggacaagtttatgtccttaggtttatgcttccctgaaacaataccacctgctattctccactttacatatcaacggcactggttctttatctaactctctggcacagcaggagtttgttttcttctgcttcagagctttgaatttactatttcagcttctaaactttatttggcaatgccttcccatggcagattccttctgtcattttgcctctgttcgaatactttctccttaatttcattcttagttaataatatctgaaattattttgttgtttaacttaattattaattttatgtatgttctacctagattataatcttcagaggaaagttttattctctgacttatttaacttaaatgcccactactttaaaaattatgacatttatttaacagatatttgctgaacaaatgtttgaaaatacatgggaaagaatgcttgaaaacacttgaaattgcttgtgtaaagaaacagttttatcagttaggatttaatcaatgtcagaagcaatgatataggaaaaatcgaggaataagacagttatggataaggagaaatcaacaaactcttaaaagatattgcctcaaaagcataagaggaaataagggtttatacatgacttttagaacactgccttggtttttggataaatggggaagttgtttgaaaacaggagggatcctagatattccttagtctgaggaggagcaattaagattcacttgtttagaggctgggagtggtggctcacgcctgtaatcccagaattttgggaggccaaggcaggcagatcacctgaggtcaagagttcaagaccaacctggccaacatggtgaaatcccatctctacaaaaatacaaaaattagacaggcatgatggcaagtgcctgtaatcccagctacttgggaggctgaggaaggagaattgcttgaacctggaaggcaggagttgcagtgagccgagatcataccactgcactccagcctgggtgacagaacaagactctgtctcaaaaaaaaaaaagagagattcaaaagattcacttgtttaggccttagcgggcttagacaccagtctctgacacattcttaaaggtcaggctctacaaatggaacccaaccagactctcagatatggccaaagatctatacacacccatctcacagatcccctatcttaaagagaccctaatttgggttcacctcagtctctataatctgtaccagcataccaataaaaatctttctcacccatccttagattgagagaagtcacttattattatgtgagtaactggaagatactgataagttgacaaatctttttctttcctttcttattcaacttttattttaacttccaaagaacaagtgcaatatgtgcagctttgttgcgcaggtcaacatgtatctttctggtcttttagccgcctaacactttgagcagatataagccttacacaggattatgaagtctgaaaggattccaccaatattattataattcctatcaacctgataggttaggggaaggtagagctctcctccaataagccagatttccagagtttctgacgtcataatctaccaaggtcatggatcgagttcagagaaaaaacaaaagcaaaaccaaacctaccaaaaaataaaaatcccaaagaaaaaataaagaaaaaaacagcatgaatacttcctgccatgttaagtggccaatatgtcagaaacagcactgagttacagataaagatgtctaaactacagtgacatcccagctgtcacagtgtgtggactattagtcaataaaacagtccctgcctcttaagagttgttttccatgcaaatacatgtcttatgtcttagaataagattccctaagaagtgaacctagcatttatacaagataattaattctaatccatagtatctggtaaagagcattctaccatcatctttaccgagcatagaagagctacaccaaaaccctgggtcatcagccagcacatacacttatccagtgataaatacacatcatcgggtgcctacatacatacctgaatataaaaaaaatacttttgctgagatgaaacaggcgtgatttatttcaaataggtacggataagtagatattgaagtaaggattcagtcttatattatattacataacattaatctattcctgcactgaaactgttgctttataggatttttcactacactaatgagaacttaagagataatggcctaaaaccacagagagtatattcaaagataagtatagcacttcttatttggaaaccaatgcttactaaatgagactaagacgtgtcccatcaaaaatcctggacctatgcctaaaacacatttcacaatccctgaacttttcaaaaattggtacatgctttaactttaaactacaggcctcactggagctacagacaagaaggtgaaaaacggctgacaaaagaagtcctggtatcttctatggtgggagaagaaaactagctaaagggaagaataaattagagaaaaattggaatgactgaatcggaacaaggcaaaggctataaaaaaaattaagcagcagtatcctcttgggggccccttccccacactatctcaatgcaaatatctgtctgaaacggtccctggctaaactccacccatgggttggccagccttgccttgaccaatagccttgacaaggcaaacttgaccaatagtcttagagtatccagtgaggccaggggccggcggctggctagggatgaagaataaaaggaagcacccttcagcagttccacacactcgcttctggaacgtctgaggttatcaataagctcctagtccagacgccatgggtcatttcacagaggaggacaaggctactatcacaagcctgtggggcaaggtgaatgtggaagatgctggaggagaaaccctgggaaggtaggctctggtgaccaggacaagggagggaaggaaggaccctgtgcctggcaaaagtccaggtcgcttctcaggatttgtggcaccttctgactgtcaaactgttcttgtcaatctcacaggctcctggttgtctacccatggacccagaggttctttgacagctttggcaacctgtcctctgcctctgccatcatgggcaaccccaaagtcaaggcacatggcaagaaggtgctgacttccttgggagatgccataaagcacctggatgatctcaagggcacctttgcccagctgagtgaactgcactgtgacaagctgcatgtggatcctgagaacttcaaggtgagtccaggagatgtttcagcactgttgcctttagtctcgaggcaacttagacaactgagtattgatctgagcacagcagggtgtgagctgtttgaagatactggggttgggagtgaagaaactgcagaggactaactgggctgagacccagtggcaatgttttagggcctaaggagtgcctctgaaaatctagatggacaactttgactttgagaaaagagaggtggaaatgaggaaaatgacttttctttattagatttcggtagaaagaactttcacctttcccctatttttgttattcgttttaaaacatctatctggaggcaggacaagtatggtcattaaaaagatgcaggcagaaggcatatattggctcagtcaaagtggggaactttggtggccaaacatacattgctaaggctattcctatatcagctggacacatataaaatgctgctaatgcttcattacaaacttatatcctttaattccagatgggggcaaagtatgtccaggggtgaggaacaattgaaacatttgggctggagtagattttgaaagtcagctctgtgtgtgtgtgtgtgtgtgtgcgcgcgtgtgtttgtgtgtgtgtgagagcgtgtgtttcttttaacgttttcagcctacagcatacagggttcatggtggcaagaagataacaagatttaaattatggccagtgactagtgctgcaagaagaacaactacctgcatttaatgggaaagcaaaatctcaggctttgagggaagttaacataggcttgattctgggtggaagcttggtgtgtagttatctggaggccaggctggagctctcagctcactatgggttcatctttattgtctcctttcatctcaacagctcctgggaaatgtgctggtgaccgttttggcaatccatttcggcaaagaattcacccctgaggtgcaggcttcctggcagaagatggtgactggagtggccagtgccctgtcctccagataccactgagctcactgcccatgatgcagagctttcaaggataggctttattctgcaagcaatcaaataataaatctattctgctaagagatcacacatggttgtcttcagttctttttttatgtctttttaaatatatgagccacaaagggttttatgttgagggatgtgtttatgtgtatttatacatggctatgtgtgtttgtgtcatgtgcacactccacacttttttgtttacgttagatgtgggttttgatgagcaaataaaagaactaggcaataaagaaacttgtacatgggagttctgcaagtgggagtaaaaggtgcaggagaaatctggttggaagaaagacctctataggacaggactcctcagaaacagatgttttggaagagatggggaaaggttcagtgaagggggctgaacccccttccctggattgcagcacagcagcgaggaaggggctcaacgaagaaaaagtgttccaagctttaggaagtcaaggtttaggcagggatagccattctattttattaggggcaatactatttccaacggcatctggcttttctcagcccttgtgaggctctacagggaggttgaggtgttagagatcagagcaggaaacaggtttttctttccacggtaactacaatgaagtgatccttactttactaaggaacttttcattttaagtgttgacgcatgcctaaagaggtgaaattaatcccatacccttaagtctacagactggtcacagcatttcaaggaggagacctcattgtaagcttctagggaggtggggacttaggtgaaggaaatgagccagcagaagctcacaagtcagcatcagcgtgtcatgtctcagcagcagaacagcacggtcagatgaaaatatagtgtgaagaatttgtataacattaattgagaaggcagattcactggagttcttatataattgaaagttaatgcacgttaataagcaagagtttagtttaatgtgatggtgttatgaacttaacgcttgtgtctccagaaaattcacatgctgaatccccaactcccaattggctccatttgtgggggaggctttggaaaagtaatcaggtttagaggagctcatgagagcagatccccatcatagaattattttcctcatcagaagcagagagattagccatttctcttccttctggtgaggacacagtgggaagtcagccacctgcaacccaggaagagagccctgaccaggaaccagcagaaaagtgagaaaaaatcctgttgttgaagtcacccagtctatgctattttgttatagcaccttgcactaagtaaggcagatgaagaaagagaaaaaaataagcttcggtgttcagtggattagaaaccatgtttatctcaggtttacaaatctccacttgtcctctgtgtttcagaataaaataccaactctactactctcatctgtaagatgcaaatagtaagcctgagcccttctgtctaactttgaattctattttttcttcaacgtactttaggcttgtaatgtgtttatatacagtgaaatgtcaagttctttctttatatttctttctttcttttttttcctcagcctcagagttttccacatgcccttcctactttcaggaacttctttctccaaacgtcttctgcctggctccatcaaatcataaaggacccacttcaaatgccatcactcactaccatttcacaattcgcactttctttctttgtcctttttttttttagtaaaacaagtttataaaaaattgaaggaataaatgaatggctacttcataggcagagtagacgcaagggctactggttgccgatttttattgttatttttcaatagtatgctaaacaaggggtagattatttatgctgcccatttttagaccataaaagataacttcctgatgttgccatggcatttttttccttttaattttatttcatttcattttaatttcgaaggtacatgtgcaggatgtgcaggcttgttacatgggtaaatgtgtgtctttctggccttttagccatctgtatcaatgagcagatataagctttacacaggatcatgaaggatgaaagaatttcaccaatattataataatttcaatcaacctgatagcttaggggataaactaatttgaagatacagcttgcctccgataagccagaattccagagcttctggcattataatctagcaaggttagagatcatggatcactttcagagaaaaacaaaaacaaactaaccaaaagcaaaacagaaccaaaaaaccaccataaatacttcctaccctgttaatggtccaatatgtcagaaacagcactgtgttagaaataaagctgtctaaagtacactaatattcgagttataatagtgtgtggactattagtcaataaaaacaacccttgcctctttagagttgttttccatgtacacgcacatcttatgtcttagagtaagattccctgagaagtgaacctagcatttatacaagataattaattctaatccacagtacctgccaaagaacattctaccatcatctttactgagcatagaagagctacgccaaaaccctgggtcatcagccagcacacacacttatccagtggtaaatacacatcatctggtgtatacatacatacctgaatatggaatcaaatatttttctaagatgaaacagtcatgatttatttcaaataggtacggataagtagatattgaggtaagcattaggtcttatattatgtaacactaatctattactgcgctgaaactgtggctttatagaaattgttttcactgcactattgagaaattaagagataatggcaaaagtcacaaagagtatattcaaaaagaagtatagcactttttccttagaaaccactgctaactgaaagagactaagatttgtcccgtcaaaaatcctggacctatgcctaaaacacatttcacaatccctgaacttttcaaaaattggtacatgctttagctttaaactacaggcctcactggagctagagacaagaaggtaaaaaacggctgacaaaagaagtcctggtatcctctatgatgggagaaggaaactagctaaagggaagaataaattagagaaaaactggaatgactgaatcggaacaaggcaaaggctataaaaaaaattagcagtatcctcttgggggccccttccccacactatctcaatgcaaatatctgtctgaaacggtccctggctaaactccacccatgggttggccagccttgccttgaccaatagccttgacaaggcaaacttgaccaatagtcttagagtatccagtgaggccaggggccggcggctggctagggatgaagaataaaaggaagcacccttcagcagttccacacactcgcttctggaacgtctgaggttatcaataagctcctagtccagacgccatgggtcatttcacagaggaggacaaggctactatcacaagcctgtggggcaaggtgaatgtggaagatgctggaggagaaaccctgggaaggtaggctctggtgaccaggacaagggagggaaggaaggaccctgtgcctggcaaaagtccaggtcgcttctcaggatttgtggcaccttctgactgtcaaactgttcttgtcaatctcacaggctcctggttgtctacccatggacccagaggttctttgacagctttggcaacctgtcctctgcctctgccatcatgggcaaccccaaagtcaaggcacatggcaagaaggtgctgacttccttgggagatgccacaaagcacctggatgatctcaagggcacctttgcccagctgagtgaactgcactgtgacaagctgcatgtggatcctgagaacttcaaggtgagtccaggagatgtttcagccctgttgcctttagtctcgaggcaacttagacaacggagtattgatctgagcacagcagggtgtgagctgtttgaagatactggggttgggggtgaagaaactgcagaggactaactgggctgagacccagtggtaatgttttagggcctaaggagtgcctctaaaaatctagatggacaattttgactttgagaaaagagaggtggaaatgaggaaaatgacttttctttattagattccagtagaaagaactttcatctttccctcatttttgttgttttaaaacatctatctggaggcaggacaagtatggtcgttaaaaagatgcaggcagaaggcatatattggctcagtcaaagtggggaactttggtggccaaacatacattgctaaggctattcctatatcagctggacacatataaaatgctgctaatgcttcattacaaacttatatcctttaattccagatgggggcaaagtatgtccaggggtgaggaacaattgaaacatttgggctggagtagattttgaaagtcagctctgtgtgtgtgtgtgtgtgtgcgcgcgcgcgtgtgtgtgtgtgtgtcagcgtgtgtttcttttaacgtcttcagcctacaacatacagggttcatggtggcaagaagatagcaagatttaaattatggccagtgactagtgcttgaaggggaacaactacctgcatttaatgggaaggcaaaatctcaggctttgagggaagttaacataggcttgattctgggtggaagcttggtgtgtagttatctggaggccaggctggagctctcagctcactatgggttcatctttattgtctcctttcatctcaacagctcctgggaaatgtgctggtgaccgttttggcaatccatttcggcaaagaattcacccctgaggtgcaggcttcctggcagaagatggtgactgcagtggccagtgccctgtcctccagataccactgagctcactgcccatgattcagagctttcaaggataggctttattctgcaagcaatacaaataataaatctattctgctgagagatcacacatgattttcttcagctcttttttttacatctttttaaatatatgagccacaaagggtttatattgagggaagtgtgtatgtgtatttctgcatgcctgtttgtgtttgtggtgtgtgcatgctcctcatttatttttatatgagatgtgcattttgatgagcaaataaaagcagtaaagacacttgtacacgggagttctgcaagtgggagtaaatggtgtaggagaaatccggtgggaagaaagacctctataggacaggacttctcagaaacagatgttttggaagagatgggaaaaggttcagtgaagacctgggggctggattgattgcagctgagtagcaaggatggttcttaaggaagggaaagtgttccaagctttaggaattcaaggtttagtcaggtgtagcaattctattttattaggaggaatactatttctaatggcacttagcttttcacagcccttgtggatgcctaagaaagtgaaattaatcccatgccctcaagtgtgcagattggtcacagcatttcaagggagagacctcattgtaagactctgggggaggtggggacttaggtgtaagaaatgaatcagcagaggctcacaagtcagcatgagcatgttatgtctgagaaacagaccagcactgtgagatcaaaatgtagtgggaagaatttgtacaacattaattggaaggcttacttaatggaatttttgtatagttggatgttagtgcatctctataagtaagagtttaatatgatggtgttacggacctaatgtttgtgtctcctcaaaattcacatgctgaatccccaactcccaactgaccttatctgtgggggaggcttttgaaaagtaattaggtttagatgagctcataagagcagatccccatcataaaattattttccttatcagaagcagagagacaagccatttctctttcctcccggtgaggacacagtgagaagtccgccatctgcaatccaggaagagaaccctgaccacgagtcagccttcagaaatgtgagaaaaaactctgttgttgaagccacccagtcttttgtattttgttatagcaccttgcactgagtaaggcagatgaagaaggagaaaaaaataagcttgggttttgagtggactacagaccatgtttatctcaggtttgcaaagctcccctcgtcccctatgtttcagtataaaatacctactctactactctcatctataagacccaaataataagcctgcgcccttctctctaactttgatttctcctatttttacttcaacatgctttactctagccttgtaatgtctttacatacagtgaaatgtaaagttctttattctttttttctttctttcttttttctcctcagcctcagaatttggcacatgcccttccttctttcaggaacttctccaacatctctgcctggctccatcatatcataaaggtcccacttcaaatgcagtcactaccgtttcagaatatgcactttctttcttttttgttttttgttttttttaagtcaaagcaaatttcttgagagagtaaagaaataaacgaatgactactgcataggcagagcagccccgagggccgctggttgttccttttatggttatttcttgatgatatgttaaacaagttttggattatttatgccttctctttttaggccatatagggtaactttctgacattgccatggcatttttcttttaatttaatttactgttaccttaaattcaggggtacacgtacaggatatgcaggtttgttttataggtaaaagtgtgccatggttttaatgggttttttttttcttgtaaagttgtttaagtttcttgtttactctggatattaggcctttgtcagaagaatagattggaaaatctttttcccattctgtagattgtctttcgctctgatggtagtttcttttgctgagcaggagctctttagtttaattagattccattggtcaatttttgcttttgctgcaattgcttttcacgctttcatcatgaaatctgtgcccgtgtttatatcatgaatagtattgccttgatttttttctaggctttttatagtttggggtttttcatttaagtctctaatccatctggagttaattttggataaggtataaggaaggagtccagtttcatttttcagcatatggctagccagttctcccccatcatttattaaattgaaaatcctttccccattgcttgcttttgtcaggtttctaaaagaccagatggttgtaggtacaatatgcagtttcttcaagtcatataataccatctgaaatctcttattaattcatttcttttagtatgtatgctggtctcctctgctcactatagtgagggcaccattagccagagaatctgtctgtctagttcatgtaagattctcagaattaagaaaaatggatggcatatgaatgaaacttcatggatgacatatggaatctaatatgtatttgttgaattaatgcataagatgcaacagagagaagttgacaactgcaatgataacctggtattgatgatataagagtctatagatcacagtagaagcaataatcatggaaaacaattggaaatggggaacagccacaaacaagaaagaatcaatacttccaggaaagtgactgcaggtcacttttcctggagcgggtgagagaaaagtggaagttagcagtaactgctgaattcctggttggctgatggaaagatggggcagctgttcactggtacgcagggttttagatgtatgtacctaaggatatgaggtatggcaatgaacagaaattcttttgggaatgagttttagggccattaaaggacatgacctgaagtttcctctgaggccagtccccacaactcaatataaatgtgtttcctgcatatagtcaaagttgccacttctttttcttcatatcatcgatctctgctcttaaagataatcttggttttgcctcaaactgtttgtcactacaaactttccccatgttcctaagtaaaacaggtaactgcctctcaactatatcaagtagactaaaatattgtgtctctaatatcagaaattcagctttaatatattgggtttaactctttgaaatttagagtctccttgaaatacacatgggggtgatttcctaaactttatttcttgtaaggatttatctcaggggtaacacacaaaccagcatcctgaacctctaagtatgaggacagtaagccttaagaatataaaataaactgttcttctctctgccggtggaagtgtgccctgtctattcctgaaattgcttgtttgagacgcatgagacgtgcagcacatgagacacgtgcagcagcctgtggaatattgtcagtgaagaatgtctttgcctgattagatataaagacaagttaaacacagcattagactatagatcaagcctgtgccagacacaaatgacctaatgcccagcacgggccacggaatctcctatcctcttgcttgaacagagcagcacacttctcccccaacactattagatgttctggcataattttgtagatatgtaggatttgacatggactattgttcaatgattcagaggaaatctcctttgttcagataagtacactgactactaaatggattaaaaaacacagtaataaaacccagttttccccttacttccctagtttgtttcttattctgctttcttccaagttgatgctggatagaggtgtttatttctattctaaaaagtgatgaaattggccgggcgcggtggctcacacctgtaatcccagcactttgggaggctgaggtgggcggatcacgaggtcaggagatcaagaccatcctggctaacatggtgaaaccccatctctactaaaaatacaaaaaattagccagagacagtggcgggtgcctgtagtcccagctactcgggaggctgaggcaggagaatggcgtgaacctgggaggcagagcttgcggtgagcagagatcgcgccactgcacactccagcctgggtgacaaagcgagactccatctcaaaaaaaaaaaaaaaaaaaagaaaaagaaagaaagaaagaaaaaaaaactgatgaaattgtgtattcaatgtagtctcaagagaattgaaaaccaagaaaggctgtggcttcttccacataaagcctggatgaataacaggataacacgttgttacattgtcacaactcctgatccaggaattgatggctaagatattcgtaattcttatccttttcagttgtaacttattcctatttgtcagcattcaggttattagcggctgctggcgaagtccttgagaaataaactgcacactggatggtgggggtagtgtaggaaaatggaggggaaggaagtaaagtttcaaattaagcctgaacagcaaagttcccctgagaaggccacctggattctatcagaaactcgaatgtccatcttgcaaaacttccttgcccaaaccccacccctggagtcacaacccacccttgaccaatagattcattttactgagggaggcaaagggctggtcaatagattcatttcactgggagaggcaaagggctgggggccagagaggagaagtaaaaagccacacatgaagcagcaatgcaggcatgcttctggctcatctgtgatcaccaggaaactcccagatctgacactgtagtgcatttcactgctgacaagaaggctgctgccaccagcctgtgaagcaaggttaaggtgagaaggctggaggtgagattctgggcaggtaggtactggaagccgggacaaggtgcagaaaggcagaaagtgtttctgaaagagggattagcccgttgtcttacatagtctgactttgcacctgctctgtgattatgactatcccacagtctcctggttgtctacccatggacctagaggtactttgaaagttttggatatctgggctctgactgtgcaataatgggcaaccccaaagtcaaggcacatggcaagaaggtgctgatctccttcggaaaagctgttatgctcacggatgacctcaaaggcacctttgctacactgagtgacctgcactgtaacaagctgcacgtggaccctgagaacttcctggtgagtagtaagtacactcacgctttcttctttacccttagatatttgcactatgggtacttttgaaagcagaggtggctttctcttgtgttatgagtcagctatgggatatgatatttcagcagtgggattttgagagttatgttgctgtaaataacataactaaaatttggtagagcaaggactatgaataatggaaggccacttaccatttgatagctctgaaaaacacatcttataaaaaattctggccaaaatcaaactgagtgtttttggatgagggaacagaagttgagatagagaaaataacatctttcctttggtcagcgaaattttctataaaaattaatagtcacttttctgcatagtcctggaggttagaaaaagatcaactgaacaaagtagtgggaagctgttaaaaagaggattgtttccctccgaatgatgatggtatacttttgtacgcatggtacaggattctttgttatgagtgtttgggaaaattgtatgtatgtatgtatgtatgtatgtgatgactggggacttatcctatccattactgttccttgaagtactattatcctactttttaaaaggacgaagtctctaaaaaaaaaatgaaacaatcacaatatgttggggtagtgagttggcatagcaagtaagagaaggataggacacaatgggaggtgcagggctgccagtcatattgaagctgatatctagcccataatggtgagagttgctcaaactctggtgaaaaaggatgtaagtgttatatctatttactgcaagtccagcttgaggccttctattcactatgtaccattttcttttttatcttcactccctccccagctcttaggcaacgtgatattgattgttttggcaacccacttcagcgaggattttaccctacagatacaggcttcttggcagtaactaacaaatgctgtggttaatgctgtagcccacaagaccactgagttccctgtccactatgtttgtacctatggtccactatgtttgtacctatgtcccaaaatctcatctcctttagatgggggaggttggggagaagagcagtatcctgcctgctgattcagttcctgcatgataaaaatagaataaagaaatatgctctctaagaaatatcattgtactctttttctgtctttatattttaccctgattcagccaaaaggacgcactatttctgatggaaatgagaatgttggagaatgggagtttaaggacagagaagatactttcttgcaatcctgcaagaaaagagagaactcgtgggtggatttagtggggtagttactcctaggaaggggaaatcgtctctagaataagacaatgtttttacagaaagggaggtcaatggaggtactctttggaggtgtaagaggattgttggtagtgtgtagaggtatgttaggactcaaattagaagttctgtataggctattatttgtatgaaactcaggatatagctcatttggtgactgcagttcacttctacttattttaaacaacatattttttattatttataatgaagtggggatggggcttcctagagaccaatcaagggccaaaccttgaactttctcttaacgtcttcaatggtattaatagagaattatctctaaggcatgtgaactggctgtcttggttttcatctgtacttcatctgctacctctgtgacctgaaacatatttataattccattaagctgtgcatatgatagatttatcatatgtattttccttaaaggatttttgtaagaactaattgaattgatacctgtaaagtctttatcacactacccaataaataataaatctctttgttcagctctctgtttctataaatatgtacaagttttattgtttttagtggtagtgattttattctctttctatatatatacacacacatgtgtgcattcataaatatatacaatttttatgaataaaaaattattagcaatcaatattgaaaaccactgatttttgtttatgtgagcaaacagcagattaaaaggctgagatttaggaaacagcacgttaagtcaagttgatagaggagaatatggacatttaaaagaggcaggatgatataaaattagggaaactggatgcagagaccagatgaagtaagaaaaatagctatcgttttgagcaaaaatcactgaagtttcttgcatatgagagtgacataataaatagggaaacgtagaaaattgattcacatgtatatatatatatagaactgattagacaaagtctaacttgggtatagtcagaggagcttgctgtaattatattgaggtgatggataaagaactgaagttgatggaaacaatgaagttaagaaaaaaaatcgagtaagagaccattgtggcagtgattgcacagaactggaaaacattgtgaaacagagagtcagagatgacagctaaaatccctgtctgtgaatgaaaagaaggaaatttattgacagaacagcaaatgcctacaagccccctgtttggatctggcaatgaacgtagccattctgtggcaatcacttcaaactcctgtacccaagacccttaggaagtatgtagcaccctcaaacctaaaacctcaaagaaagaggttttagaagatataataccctttcttctccagtttcattaatcccaaaacctctttctcaaagtatttcctctatgtgtccaccccaaagagctcacctcaccatatctcttgagtgggagcacatagataggcggtgctaccatctaacagcttctgaaattcctttgtcatatttttgagtccccactaataacccacaaagcagaataaataccagttgctcatgtacaataatcactcaactgctgtcttgtagcatacattaattaagcacattctttgaataattactgtgtccaaacaatcacactttaaaatctcacacttgtgctatcccttgcccttctgaatgtcactctgtattttaaatgaagagatgagggttgaatttcctgtgttacttattgttcatttctcgatgaggagttttcacattcacctttagtggaaaacacataagtacacatcttacaggaaaaatataccaaactgacatgtagcatgaatgcttgtgcatgtagtcatataaaatcttgtagcaatgtaaacattctctgatatacacatacagatgtgtctatatgtctacacaatttcttatgctccatgaacaaacattccatgcacacataagaacacacactgttacagatgcatacttgagtgcattgacaaaattaccccagtcaatctagagaatttggatttctgcatttgactctgttagctttgtacatgctgttcatttactctgggtgatgtctttccctcattttgccttgtctatcttgtactcatactttaagtcctaacttatatgttatctcaactaagaagctatttttttttaattttaactgggcttaaagccctgtctataaactctgctacaattatgggctctttcttataatatttagtgtttttcctactaatgtacttaatctgctcattgtatattcctaccactaaattttaacctcttttatggtagagacattgtcttgtaaactcttatttccctagtatttggagatgaaaaaaaagattaaattatccaaaattagatctctcttttctacattatgagtattacactatccatagagaagtttgtttgagacctaaactgaggaacctttggttctaaaatgactatgtgatatcttagtatttataggtcatgaggttccttcctctgcctctgctatagtttgattagtcaacaagcatgtgtcatgcatttattcacatcagaatttcatacactaataagacatagtatcagaagtcagtttattagttatatcagttagggtccatcaaggaaaggacaaaccattatcagttactcaacctagaattaaatacagctcttaatagttaattatccttgtattggaagagctaaaatatcaaataaaggacagtgcagaaatctagatgttagtaacatcagaaaacctcttccgccattaggcctagaagggcagaaggagaaaatgtttataccaccagagtccagaaccagagcccataaccagaggtccactggattcagtgagctagtgggtgctccttggagagagccagaactgtctaatgggggcatcaaagtatcagccataaaaaaccataaaaaagactgtctgctgtaggagatccgttcagagagagagagagaccagaaataatcttgcttatgctttccctcagccagtgtttaccattgcagaatgtacatgcgactgaaagggtgaggaaacctgggaaatgtcagttcctcaaatacagagaacactgagggaaggatgagaaataaatgtgaaagcagacatgaatggtaattgacagaaggaaactaggatgtgtccagtaaatgaataattacagtgtgcagtgattattgcaatgattaatgtattgataagataatatgaaaacacagaattcaaacagcagtgaactgagattagaattgtggagagcactggcatttaagaatgtcacacttagaatgtgtctctaggcattgttctgtgcatatatcatctcaatattcattatctgaaaattatgaattaggtacaaagctcaaataatttattttttcaggttagcaagaactttttttttttttttctgagatagagcattgctatggttgcccaggctggagtgcaatggcatgatccaggctcactgcaacatctgcctcccaggttcaagcgattctcctgcctcagcctcccaagtagctggcactacaggcatgtgccaccaccatgcctggctaattttctatttttagtagatagggggtttcaccatgttggtcaggctgatctcgaactcctaacatcaggtgatccaccctcctcggcctctgaaagtgctgggatcacaggcgtgagccaccacacccagccaagaatgtgaattttgtagaaggatataacccatatttctctgaccctagagtccttagtatacctcccataccatgtggctcatcctccttacatacatttcccatctttcaccctaccttttcctttttgtttcagcttttcactgtgtcaaaatctagaaccttatctcctacctgctctgaaaccaacagcaagttgacttccattctaacccacattggcattacactaattaaaatcgatactgagttctaaaatcatcggggattttggggactatgtcttacttcatacttccttgagatttcacattaaatgttggtgttcattaaaggtccttcatttaactttgtattcatcacactcttggattcacagttatatctaaactcttaaatacagcctgtataatcccaattcccaactctgatttctaacctctgacctccaacctcagtgccaaacccatatatcaaacaatgtactgggcttatttatatagatgtcctataggcacctcagactcagcatgggtatttcacttgttatactaaaactgtttctcttccagtgttttccattttagtcattagatagctacttgcccattcaccaaggtcacagattaaaatcatttccctacctctaatcaacagttcgattctgcttcaatttgtccctatctattaatcaccactcttactgcccagtcaggtcctcattgtttcctgaacaagagtagatgctattctttccacttttagaccttatcctggctggatgcggtggctcaggcttgtaaacccagcactttgggaggccaaggcaggcagatcacttgaggtcaggagttcaagaccagcctgaccaacatggtgaaaccccatctctactaaaaatacaaaatcagccgggcgtgtggtgcatgcctgcagtcccagctattcaggtggctgaggcaggagaattgcttgaacccaggaggcagaggttgcggtgagcctagattgcaccattgcactctagcttgggcaatagggatgaaactccatctcagaagagaaaagaaaaaaagaccttattctgttatacaaatcctctcaatgcaatccatatagaataaacatgtaaccagatctcccaatgtgtaaaatcatttcaggtagaacagaattaaagtgaaaagccaagtctttggaattaacagacaaagatcaaataacagtcctcatggccttaagaatttacctaacattttttttagaatcaattttcttatatatgaattggaaacataattcctccctcacaaacacattctaagattttaaggagatattgatgaagtacatcatctgtcatttttaacaggtagtggtagtgattcacacagcacattatgatctgttcttgtatgttctgttccattctgtattcttgacctggttgtattctttctgagctccagatccacatatctaagtacatctttttgcattttacaagagtgcatacaatacaatgtatccaagactgtatttctgattttatcgtaccactaaactcacaaatgtggccctattcttgtgttcacgactgacatcaccgtcatggtccaagtctgataatagaaatggcattgtcactttcttccctactgcaacagaagcccagctatttgtctcccattttctctacttctaaaatacatttcttcactaagtgagaataatcttttaaagacacaaatcaaaccatgccaccacctttcttgaattattcaatatctttcgttggcttccaggttacagaaaaataacttgtaacaaagtttaaaggtcattcatggctcctctctaccctattttataacatttccccttgtgatcagaatctcaggcacatcatccatctttctatatacaaataaagtcatatagtttgaactcacctctggttacttttaatcaaccaaatgctgtaaaatgcatttgtatcgctacgtgttaagcagtagttgattcttttcatttctgtgtaatattctattctttgactataccgtaatttatcaattctactgttggtaagcatttaagtggctaccggtttgaggtttttatgattattgctgtcataagcatttctatacatgtctttggatacacacatgcatgtgtttctgaatatctaaaaatgtaattgctaggtaatagacttatcaagcatccagcatttgtggatactattaaaggttttccaaaggggttatactattgtacagtgtcaccaacagagtttgagtttctattgatccatatcaccaccaaaatttgaactgtcagtcttatctcttctcttgtctcttttttcctcttttttttccttcccttcccctctcttcgtttcttttctctcctcttctcttctttcctctcttcccttccctttctctttctcttccctatcccttctcctctcctctcccctccttttttctcctctcctctccattatttatttttccttcttctcctccatcccttccatcctctctcttcccctcttccttccttcctttctccatttcttcctcctctttccttcaatccttccttttggatatgctcatgggtgtgtatttgtctgccattgtggcattatttgaattcagaaaagagtgaaaaactactgggatcttcattcctgggtctaattccacatttttttttaagaacacatctgtaaaaatgttctgtactagcatattcccaggaacttcgttaaatttaatctggctgaatatggtaaatctacttttcactttgcattctttctttagtcataccataattttaaacattcaaaatatttgtatataatatttgattttatctgtcattaaaatgttaaccttaaaattcatgtttccagaacctatttcaataactggtaaataaacactattcattttttaaatattcttttaatggatatttatttcaatataataaaaaattagagttttattataggaagaatttaccaaaagaaggaggaagcaagcaagtttaaactgcagcaatagatttgtccattccaacctctcaaaattcccttggagacaaaaatctctagaggcaaagaagaactttatattgagtcaacttgttaaaacatctgcttttagataagttttcttagtataaagtgacagaaacaaataagttaaactctaagatacattccactatattagcctaaaacacttctgcaaaaatgaaactaggaggatatttttagaaacaactgctgaaagagatgcggtggggagatatgtagaggagaacagggtttctgagtcaagacacacatgacagaacagccaatctcagggcaagttaagggaatagtggaatgaaggttcatttttcattctcacaaactaatgaaaccctgcttatcttaaaccaacctgctcactggagcagggaggacaggaccagcataaaaggcagggcagagtcgactgttgcttacactttcttctgacataacagtgttcactagcaacctcaaacagacaccatggtgcatctgactcctgaggagaagactgctgtcaatgccctgtggggcaaagtgaacgtggatgcagttggtggtgaggccctgggcaggttggtatcaaggttataagagaggctcaaggaggcaaatggaaactgggcatgtgtagacagagaagactcttgggtttctgataggcactgactctctgtcccttgggctgttttcctaccctcagattactggtggtctacccttggacccagaggttctttgagtcctttggggatctgtcctctcctgatgctgttatgggcaaccctaaggtgaaggctcatggcaagaaggtgctaggtgcctttagtgatggcctggctcacctggacaacctcaagggcactttttctcagctgagtgagctgcactgtgacaagctgcacgtggatcctgagaacttcagggtgagtccaggagatgcttcacttttctctttttactttctaatcttacattttggttcttttacctacctgctcttctcccacatttttgtcattttactatattttatcatttaatgcttctaaaattttgttaattttttatttaaatattctgcattttttccttcctcacaatcttgctattttaaattatttaatatcctgtctttctctcccaaccccctcccttcatttttccttctctaacaacaactcaaattatgcataccagctctcacctgctaattctgcacttagaataatccttttgtctctccacatgggtatgggagaggctccaactcaaagatgagaggcatagaatactgttttagaggctataaatcattttacaataaggaataattggaattttataaattctgtagtaaatggaatggaaaggaaagtgaatatttgattatgaaagactaggcagttacactggaggtggggcagaagtcgttgctaggagacagcccatcatcacactgattaatcaattaatttgtatctattaatctgtttatagtaattaatttgtatatgctatatacacatacaaaattaaaactaatttggaattaatttgtatatagtattatacagcatatatagcatatatgtacatatatagactacatgctagttaagtacatagaggatgtgtgtgtatagatatatgttatatgtatgcattcatatatgtacttatttatgctgatgggaataacctggggatcagttttgtctaagatttgggcagaaaaaaatgggtgttggctcagtttctcagaagccagtctttatttctctgttaaccatatgcatgtatctgcctacctcttctccgcagctcttgggcaatgtgctggtgtgtgtgctggcccgcaactttggcaaggaattcaccccacaaatgcaggctgcctatcagaaggtggtggctggtgtggctaatgccctggctcacaagtaccattgagatcctggactgtttcctgataaccataagaagaccctatttccctagattctattttctgaacttgggaacacaatgcctacttcaagggtatggcttctgcctaataaagaatgttcagctcaacttcctgattaatttcacttatttcatttttttgtccaggtgtgtaagaaggttcctgaggctctacagatagggagcacttgtttattttacaaagagtacatgggaaaagagaaaagcaagggaaccgtacaaggcattaatgggtgacacttctacctccaaagagcagaaattatcaagaactcttgatacaaagataatactggcactgcagaggttctagggaagacctcaaccctaagacatagcctcaagggtaatgctacgattaaactccaacaattactgagaaaataatgtgctcaattaaaggcataatgattactcaagacaatgttatgttgtctttcttcctccttcctttgcctgcacattgtagcccataatactataccccatcaagtgttcctgctccaagaaatagcttcctcctcttacttgccccagaacatctctgtaaagaatttcctcttatcttcccatatttcagtcaagattcattgctcacgtattacttgtgacctctcttgaccccagccacaataaacttctctatactacccaaaaaatctttccaaaccctcccccacaccattttttatatttttatatttttcttatttatttcatgcacacacacacactccgtgctttataagcaattctgcctattctctaccttcttacatgcctactgtgcctcatattaaattcatcaatgggcagaaagaaaatatttattcaagaaaacagtgaatgaatgaacgaatgagtaaatgagtaaatgaaggaatgattattccttgctttagaacttctggaattagaggacaatattaataataccatcgcacagtgtttctttgttgttaatgctacaacatacaaagaggaagcatgcagtaaacaaccgaacagttatttcctttctgatcataggagtaatatttttttccttgagcaccatttttgccataggtaaaattagaaggatttttagaactttctcagttgtatacatttttaaaaatctgtattatatgcatgttgattaattttaaacttacttgaatacctaaacagaatctgttgtttccttgtgtttgaaagtgctttcacagtaactctgtctgtactgccagaatatactgacaatgtgttatagttaactgttttgatcacaacattttgaattgactggcagcagaagctcttttatatccatgtgttttccttaagtcattatacatagtaggcactgagaactctttatatctgaataagatatttaggaaccactggtttacatatcagaagcagagctactcagggcattttggggaagatcactttcacattcctgagcatagggaagttctcataagagtaagatattaaaaggagatacttgtgtggtattcgaaagacagtaagagagattgtagaccttatgatcttgatagggaaaacaaactacattcctttctccaaaagtcaaaaaaaaagagcaaatatagcttactataccttctattcctacaccattagaagtagtcagtgagtctaggcaagatgttggccctaaaaatccaaataccagagaattcatgagaacatcacctggatgggacatgtgccgagcacacacaattactatatgctaggcattgctatcttcatattgaagatgaggaggtcaagagatgaaaaaagacttggcaccttgttgttatattaaaattatttgttagagtagagcttttgtaagagtctaggagtgtgggagctaaatgatgatacacatggacacaaaaaatagatcaacagacacccaggcctacttgagggttgagggtgggaagagggagacgatgaaaaagaacctattgggtattaagttcatcactgagtgatgaaataatctgtacatcaagacccagtgatatgcaatttacctatataacttgtacatgtacccccaaatttaaaatgaaagttaaaacaaagtataggaatggaattaattcctcaagatttggctttaattttatttgataatttatcaaatggttgtttttcttttctcactatggcgttgctttataaactatgttcagtatgtctgaatgaaagggtgtgtgtgtgtgtgaaagagagggagagaggaagggaagagaggacgtaataatgtgaatttgagttcatgaaaatttttcaataaaataatttaatgtcaggagaattaagcctaatagtctcctaaatcatccatctcttgagcttcagagcagtcctctgaattaatgcctacatgtttgtaaagggtgttcagactgaagccaagattctacctctaaagagatgcaatctcaaatttatctgaagactgtacctctgctctccataaattgacaccatggcccacttaatgaggttaaaaaaaagctaattctgaatgaaaatctgagcccagtggaggaaatattaatgaacaaggtgcagactgaaatataaatttttctgtaataattatgcatatactttagcaaagttctgtctatgttgactttattgctttttggtaagaaatacaactttttaaagtgaactaaactatcctatttccaaactattttgtgtgtgtgcggtttgtttctatgggttctggttttcttggagcatttttatttcattttaattaattaattctgagagctgctgagttgtgtttactgagagattgtgtatctgcgagagaagtctgtagcaagtagctagactgtgcttgacctaggaacatatacagtagattgctaaaatgtctcacttggggaattttagactaaacagtagagcatgtataaaaatactctagtcaagtgctgcttttgaaacaaatgataaaaccacactcccatagatgagtgtcatgattttcatggaggaagttaatattcatcctctaagtatacccagactagggccattctgatataaaacattaggacttaagaaagattaatagactggagtaaaggaaatggacctctgtctctctcgctgtctcttttttgaggacttgtgtgtgtgtgtgtgtgtgtgtgtgtgtgtgtgttgtggtcagtggggctggaataaaagtagaatagacctgcacctgctgtggcatccattcacagagtagaagcaagctcacaatagtgaagatgtcagtaagcttgaatagtttttcaggaactttgaatgctgatttagatttgaaactgaggctctgaccataaccaaatttgcactatttattgcttcttgaaacttatttgcctggtatgcctgggcttttgatggtcttagtatagcttgcagccttgtccctgcagggtattatgggtaatagaaagaaaagtctgcgttacactctagtcacactaagtaactaccattggaaaagcaacccctgccttgaagccaggatgatggtatctgcagcagttgccaacacaagagaaggatccatagttcatcatttaaaaaagaaaacaaaatagaaaaaggaaaactatttctgagcataagaagttgtagggtaagtctttaagaaggtgacaatttctgccaatcaggatttcaaagctcttgctttgacaattttggtctttcagaatactataaatataacctatattataatttcataaagtctgtgcattttctttgacccaggatatttgcaaaagacatattcaaacttccgcagaacactttatttcacatatacatgcctcttatatcagggatgtgaaacagggtcttgaaaactgtctaaatctaaaacaatgctaatgcaggtttaaatttaataaaataaaatccaaaatctaacagccaagtcaaatctgcatgttttaacatttaaaatattttaaagacgtcttttcccaggattcaacatgtgaaatcttttctcagggatacacgtgtgcctagatcctcattgctttagttttttacagaggaatgaatataaaaagaaaatacttaaattttatccctcttacctctataatcatacataggcataattttttaacctaggctccagatagccatagaagaaccaaacactttctgcgtgtgtgagaataatcagagtgagattttttcacaagtacctgatgagggttgagacaggtagaaaaagtgagagatctctatttatttagcaataatagagaaagcatttaagagaataaagcaatggaaataagaaatttgtaaatttccttctgataactagaaatagaggatccagtttcttttggttaacctaaattttatttcattttattgttttattttattttattttattttattttgtgtaatcgtagtttcagagtgttagagctgaaaggaagaagtaggagaaacatgcaaagtaaaagtataacactttccttactaaaccgacatgggtttccaggtaggggcaggattcaggatgactgacagggcccttagggaacactgagaccctacgctgacctcataaatgcttgctacctttgctgttttaattacatcttttaatagcaggaagcagaactctgcacttcaaaagtttttcctcacctgaggagttaatttagtacaaggggaaaaagtacagggggatgggagaaaggcgatcacgttgggaagctatagagaaagaagagtaaattttagtaaaggaggtttaaacaaacaaaatataaagagaaataggaacttgaatcaaggaaatgattttaaaacgcagtattcttagtggactagaggaaaaaaataatctgagccaagtagaagaccttttcccctcctacccctactttctaagtcacagaggctttttgttcccccagacactcttgcagattagtccaggcagaaacagttagatgtccccagttaacctcctatttgacaccactgattaccccattgatagtcacactttgggttgtaagtgactttttatttatttgtatttttgactgcattaagaggtctctagttttttatctcttgtttcccaaaacctaataagtaactaatgcacagagcacattgatttgtatttattctatttttagacataatttattagcatgcatgagcaaattaagaaaaacaacaacaaatgaatgcatatatatgtatatgtatgtgtgtatatatacacacatatatatatatattttttcttttcttaccagaaggttttaatccaaataaggagaagatatgcttagaaccgaggtagagttttcatccattctgtcctgtaagtattttgcatattctggagacgcaggaagagatccatctacatatcccaaagctgaattatggtagacaaaactcttccacttttagtgcatcaacttcttatttgtgtaataagaaaattgggaaaacgatcttcaatatgcttaccaagctgtgattccaaatattacgtaaatacacttgcaaaggaggatgtttttagtagcaatttgtactgatggtatggggccaagagatatatcttagagggagggctgagggtttgaagtccaactcctaagccagtgccagaagagccaaggacaggtacggctgtcatcacttagacctcaccctgtggagccacaccctagggttggccaatctactcccaggagcagggagggcaggagccagggctgggcataaaagtcagggcagagccatctattgcttacatttgcttctgacacaactgtgttcactagcaacctcaaacagacaccatggtgcatctgactcctgaggagaagtctgccgttactgccctgtggggcaaggtgaacgtggatgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcagggtgagtctatgggacgcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatctctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcatatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacaggggtcatggtttgactgtcctgtgagcccttcttccctgcctcccccactcacagtgacccggaatctgcagtgctagtctcccggaactatcactctttcacagtctgctttggaaggactgggcttagtatgaaaagttaggactgagaagaatttgaaaggcggctttttgtagcttgatattcactactgtcttattaccctgtcataggcccaccccaaatggaagtcccattcttcctcaggatgtttaagattagcattcaggaagagatcagaggtctgctggctcccttatcatgtcccttatggtgcttctggctctgcagttattagcatagtgttaccatcaaccaccttaacttcatttttcttattcaatacctaggtaggtagatgctagattctggaaataaaatatgagtctcaagtggtccttgtcctctctcccagtcaaattctgaatctagttggcaagattctgaaatcaaggcatataatcagtaataagtgatgatagaagggtatatagaagaattttattatatgagagggtgaaaccctcaaaatgaaatgaaatcagacccttgtcttacaccataaacaaaaataaatttgaatgggttaaagaattaaactaagacctaaaaccataaaaatttttaaagaaatcaaaagaagaaaattctaatattcacgttgcagccgttttttgaatttgatatgagaagcaaaggcaacaaaaggaaaaataaagaagtgaggctacatcaaactaaaaaatttccacacaaaaaacaaaacaatgaacaaatgaaaggtgaaccatgaaatggcatatttgcaaaccaaatatttcttaaatattttggttaatatccaaaatatataagaaacacagatgattcaataacaaacaaaaaattaaaaataggaaaataaaaaaattaaaaagaagaaaatcctgccatttatggcagaattgatgaacctggaggatgtaaaactaagaaaaataagcctgacacaaaaagacaaatactacacaaccttgctcatatgtgaaacataaaaaagtcactctcatggaaacagacagtagaggtatggtttccaggggttgggggtgggagaatcaggaaactattactcaaagggtataaaatttcagttatgtgggatgaataaattctagatatctaatgtacagcatcgtgactgtagttaattgtactgtaagtatatttaaaatttgcaaagagagtagatttttttttttttttagatggagttttgctcttgttgtccaggctggagtgcaatggcaagatcttggctcactgcaacctccgcctcctgggttcaagcaaatctcctgcctcagcctcccgagtagctgggattacaggcatgcgacaccatgcccagctaattttgtatttttagtagagacggggtttctccatgttggtcaggctgatccgcctgcctcggccacccaaagggctgggattacaggcgtgagccaccgggcctggccgagagtagatcttaaaagcatttaccacaagaaaaaggtaactatgtgagataatgggtatgttaattagcttgattgtggtaatcatttcacaaggtatacatatattaaaacatcatgttgtacaccttaaatatatacaatttttatttgtgaatgatacctcaataaagttgaagaataataaaaaagaatagacatcacatgaattaaaaaactaaaaaataaaaaaatgcatcttgatgattagaattgcattcttgatttttcagatacaaatatccatttgactgtttactcttttccaaaacaatacaataaattttagcactttatcttcattttccccttcccaatctataattatatatatatatattttagatattttgtatagttttactccctagattttctagtgttattattaaatagtgaagaaatgtttacacttatgtacaaaatgttttgcatgcttttcttcatttctaacattctctctaagtttattctatttttttctgattatccttaatattatctctttctgctggaaatacattgttacttttggtttatctaaaaatggcttcattttcttcattctaaaatcatgttaaattaataccactcatgtgtaagtaagatagtggaataaatagaaatccaaaaactaaatctcactaaaatataataatgtgatatataaaaatatagcttttaaatttagcttggaaataaaaaacaaacagtaattgaacaactatactttttgaaaagagtaaagtgaaatgcttaactgcatataccacaatcgattacacaattaggtgtgaaggtaaaattcagtcacgaaaaaactagaataaaaatatgggaagacatgtatataatcttagagataacactgttatttaattatcaacccaaagtagaaactatcaagggagaaataaattcagtcaacaataaaagcatttaagaagttattctaggctgggagcggtggctcacacctgcaattgcagcactttgggaggcctagacaggcggatcacgacgtcaggagttcaagatcagcctggccaacatagtgaaacctcatcgctactaaaaatataaaaacttagcctggcgtggtggcaggcatgtgtaatcccagcaatttgggaggctgaggcaggagaatcgcttgatcctgggaggcagaggttgcagtgagccaagattgtgccactgcattccagcccaggtgacagcatgagactccgtcacaaaaaaaaaagaaaaaaaaaaggggggggggagcggtggagccaagatgaccgaataggaacagctccagtctatagctcccatcgtgagtgacgcagaagacgggtgatttctgcatttccaactgaggtaccaggttcatctcacagggaagtgccaggcagtgggtgcaggacagtaggtgcagtgcactgtgcatgagccaaagcagggcgaggcatcacctcacccgggaagcacaaggggtcagggaattccctttcctagtcaaagaaaagggtgacagatggcacctggaaaatcgggtcactcccgccctaatactgcgctcttccaacaagcttaacaaatggcacaccaggagattatatcccatgcctggctcagagggtcctacgcccatggagcctcgctcattgctagcacagcagtctgaggtcaaactgcaaggtggcagtgaggctgggggaggggtgcccaccattgtccaggcttgagcaggtaaacaaagccgcctggaagctcgaactgggtggagcccaccacagctcaaggaggcctgcctgcctctgtaggctccacctctaggggcagggcacagacaaacaaaagacaacaagaacctctgcagacttaaatgtccctgtctgacagctttgaagagagtagtggttctcccagcacatagcttcagatctgagaacaggcagactgcctcctcaagtgggtccctgacccccgagtagcctaactgggaggcatcccccagtaggggcagactgacacctcacatggctggtactcctctaagacaaaacttccagaggaatgatcaggcagcagcatttgcggttcaccaatatccactgttctgcagccaccgctgttgatacccaggaaaacagcttctggagtggacctccagtaaactccaacagacctgcagctgagggtcctgactgttagaaggaaaactaacaaacagaaaggacatccacaccaaaaacccatctgtacatcgccatcatcaaagaccaaaggtagataaaaccataaagatggggaaaaagcagagcagaaaaactggacactctaaaaatgagagtgcctctcctcctccaaagtaacgcagctcctcaccagcaatggaacaaagctgggcagagaatgactttgacgagttgagagaggaaggcttcagaagatcaaactactccaagctaaaggaggaagttcgaacaaacggcaaagaagtaaaaaactttgaaaaaaaattagatgaatggataactagaataaccaatgcacagaagtccttaaaggacctgatggagctgaaaaccaaggcaggagaactacgtgacaaatacacaagcctcagtaaccgatgagatcaactggaagaaagggtatcaatgacgaaagatgaaatgaatgaaatgaagcatgaagagaagtttagagaaaaaagaataaaaagaaacgaacaaagcctccaagaaatatgggactatgtgaaaagaccaaatctacatctaattggtgtagctgaaagtgatggggagaatggaaccaagttggaaaacactctgcaggatattatccaggagaacttccccaatctagcaaggcaagcccaaattcacattcaggaaatacagagaacgccacaaagatactcctagagaaaagcaactccaagacacataactgtcagattcaccaaagttgaaatgaaggaaaaaatgttaagggcagccagagagaaaggtcgggttacccacaaagggaagcccatcagactaacagctgatctatcggcagaaactctacaagccagaagaaagtgggggccaatattcaacattgttaaagaaaagaattttcaacccagaatttcatatccagccaaactaagcttcataagtgaaggagaaataaaatcctttacagacaagcaaatgctgagagattttgtcaccaccaggcctgccctacaagagctcctgaaggaagcactaaacatggaaaggaacaactagtatcagccactgcaaaaacatgccaaattgtaaagaccatcaaggctaggaagaaactgcatcaacgagcaaaataaccagctaacatcataatgacaggatcaaattcatacataacaatactcaccttaaatgtaaataggctaaatgctccaattaaaagacacagactggcaaattggataaggagtcaagacccatctgtgttctgtattcaggaaacccatctcacgtgcagagacacacataggctcgaaataaaaggatggaggaatatctaccaagcaaatggaaaacaaaaaaaggcaggggttgcaatcctagtctctgataaaacagattttaaaccaacaaagatcaaaagagacaaagaaggccattacataatggcaaagggatctattcaagaagaagaactaactatactaaatatatatgcacccaatacaggagcacccagattcataaaacaagtcctgagtgacctacaaagagacttagatgcccacacaataataatgggagactttaacaccccactgtcaacattagacagatcaacgagacagaaagttaacaaggatatccaggaattggactcagctctgcaccaagcagacctaatagacatctacagaactctccaccccaaatcaacagaatatacattcttttcagcaccacaccacacctattccaaaactgaccacatagttggaagtaaagctctcctcagcaaatgtaaaagaacagaaactataacaaactgtctctcagaccacagtgcaatcaaactagaactcaggattaagaaactcactcaaaaccactcagctacatggaaactgaacagcctgctcctgaatgactactgggtacataacaaaatgaaggcagaaataaagatgttctttgaaaccaacgagaacaaagacacaacacaccagaatctctgagacacattcaaagcagtgtgtagagggaaatttatagcactaaatgcccacaagggaaagcaggaaagatctaaaattgacaccctaacatcacaattaaaaaactagagaagcaggagcaaacacattcaaaagctaacagaagacaagaaataactaagatcagagcagaagtgaaggacatagagacacaaaaaaacccttcaaaaaaatcaatgaatccagaagctgtttttttgaaaagatcaacaaaattgatagactgctagcaagactaataaagaagaaaagagagaagaatcaaatagacgcaataaaaaatgacacggggtatcaccactgatcccacagaaatacaaactaccgtcagagaatactataaacacctctacgcaaataaactagaaaatctagaagaaatggataaattcctcgacacatacactctgccaagactaaaccaggaagaagttgtatctctgaatagaccaataacaggctctgaaattgaggcaataattaatagcttatcaaccaaaaaaagtccgggaccagtaggattcatagccgaattctaccagaggtacaaggaggagctggtaccattccttctgaaactattccaatcaatagaaaaagagggaatcctccctaactcattttatgaggccagcatcatcctgataccaaagcctgacagagacacaacaaaaaaagagaatgttacaccaatatccttgatgaacattgatgcaaaaatcctcaataaaatactggcaaactgatccaccatgatcaagtgggcttcatccctgccatgcaaggctggttcaacatacgaaaatcaataaacataatccagcatataaacagaaccaaagacacaaaccatatgattatctcaatagatgcagaaaaggcctttgacaaaattcaacaacgcttcatgctaaaaactctcaataaattaggtattgatgggacatatctcaaaataataagagctatctatgacaaacccacagccaatatcatactgagtggacaaaaactggaagcattccctttgaaaactggcacaaggcagggatgccctctctcaccactcctattcaacatagtgttgtaagttctggccagggcaatcaggcaggagaaggaaataaagggcattcaattaggaaaagaggaagtgaaattgtccctgtttgcagatgacatgattgtatatctagaaaaccccattgtctcagcccaaaatctccttaagctgataagcaacttcagcaaagtctcaggatataaaatcagtgtgcaaaaatcacaagtattcctatgcaccaataacagacaaacagagagccaaatcatgagtgaactcccattcacaattgcttcaaagagaataaaatacctaggaatccaacttacaagggatgtgaaggacctcttcaaggagaactacaaaccactgctcaatgaaataaaagaggatacaaacaaatggaagaacattccatgctcatgggtaggaagaatcaatatcgtgaaaatggtcatactgcccaaggtaatttatagattcaatgccatccccatcaagctaccaatgactttcttcacagaactggaaaaaactactttaaagttcatatggaaccaaaaaagagcccacatcaccaaggcaatcctaagccaaaagaacaaagctggaggcatcacgctacctgacttcaaactatactacaatgctacggtaaccaaaacagcatggtactggtaccaaaacagagatctagaccaatggaacagaacagagccctcagaaataatgccgcatatctacaactatctgatctttgacaaacctgagagaaacaagcaatggggaaaggattccctatttaataaatggtgctgggaaaactggctagccatatgtagaaagctgaaactggatcccttccttacaccttatacaaaaattaattcaagatggattaaagacttacatgttagacctaaaaccataaaaaccctagaaaaaaacctaggcaataccattcaggacataggcatgggcaaggacttcatgtctaaaacaccaaaagcaatggcaacaaaagacaaaatggacaaacgggatctaattaaactaaagagcttctgcacagctaaagaaactaccatcagagtgaacaggcaacctacaaaatgggagaaaatttttgcaatctactcatctgacaaagggctaatatccagaatctacaatgaactcaaacaaatttacaagaaaaaacaaacaaccccatcaaaaagtgggcaaaggatatgaacagacacttcgcaaaagaagacatttatgtaatcaaaaaacacatgaaaaaatgctcatcatcactagccatcagagaaatgcaaatcaaaaccacaatgagataccatctcacaccagttagaatggcgatcattaaaaagtcaggaaacaacaggtgctggagaggatgtggagaaacaggaacaacttttacactgttggtgggactgtaaactagttcaaccattgcggaagtcagtgtggcaattcctcaggaatctagaactagaaataccatttgacccagccatcccattactgggtacatacccaaaggattataaatcatgctgctataaagacacatgcacacgtatgtttattgcagcactattcacaatagcaaagacttggaaccaacccaaatgtccaacaacgatagactggattaagaaaatgtggcacatatacaccatggaatactatgcagccataaaaaatgatgagttcatgtcctttgtagggacatggatgaagctggaaactatcattctcagcaaactatcacaaggagaataaaccaaacaccgcatgttctcactcataggtgggaattgaacaatgagaacacatggacacatgaagaggaacatcacactctggggactgttatggggtggggggcaggggcagggatagcactaggagatatacctaatgctaaatgacgagttaatgggtgcagcacaccaacatggcacatgtatacatatataacaaacctgcatgttgtgcacatgtaccctaaaacttgaagtataataataaaaaaaagttatcctattaaaactgatctcacacatccgtagagccattatcaagtctttctctttgaaatagacagaaatttagtgttttctcagtcagttaac

SEQ ID NO: 20 - DNA, 388 bp, Homo sapiens
  source 1..388: mol_type = genomic DNA
taagcttcagtttttccttagttcctgttacatttctgtgtgtctccattagtgacctcccatagtccaagcatgagcagttctggccaggcccctgtcggggtcagtgccccacccccgccttctggttctgtgtaaccttctaagcaaaccttctggctcaagcacagcaatgctgagtcatgatgagtcatgctgaggcttagggtgtgtgcccagatgttctcagcctagagtgatgactcctatctgggtccccagcaggatgcttacagggcagatggcaaaaaaaaggagaagctgaccacctgactaaaactccacctcaaacggcatcataaagaaaatggatgcctgagacagaatgtgacatattctagaatatatt

SEQ ID NO: 21 - DNA, 387 bp, Homo sapiens
  source 1..387: mol_type = genomic DNA
taagcttcagtttttccttagttcctgttacatttctgtgtgtctccattagtgacctcccatagtccaagcatgagcagttctggccaggcccctgtcggggtcagtgccccacccccgccttctggttctgtgtaaccttctaagcaaaccttctggctcaagcacagcaatgctgagtcatgatgagtcatgctgaggctagggtgtgtgcccagatgttctcagcctagagtgatgactcctatctgggtccccagcaggatgcttacagggcagatggcaaaaaaaaggagaagctgaccacctgactaaaactccacctcaaacggcatcataaagaaaatggatgcctgagacagaatgtgacatattctagaatatatt

SEQ ID NO: 22 - DNA, 286 bp, Homo sapiens
  source 1..286: mol_type = genomic DNA
ctgagcaactaactcatgcaggactctcaaacactaacctatagccttttctatgtatctacttgtgtagaaaccaagcgtggggactgagaaggcaatagcaggagcattctgactctcactgcctttggctaggtccctccctcatcacagctcagcatagtccgagctcttatctatatccacacacagtttctgacgctgcccagctatcaccatcccaagtctaaagaaaaaaataatgggtttgcccatctctgttgattagaaaacaaaacaaaataaa

SEQ ID NO: 23 - DNA, 286 bp, Homo sapiens
  source 1..286: mol_type = genomic DNA
ctgagcaactaatcatgcaggactctcaaacactaacctatagccttttctatgtatctacttgtgtagaaaccaagcgtggggactgagaaggcaatagcaggagcattctgactctcactgcctttagctaggcccctccctcatcacagctcagcatagtcctgagctcttatctatatccacacacagtttctgacgctgcccagctatcaccatcccaagtctaaagaaaaaaataatgggtttgcccatctctgttgattagaaaacaaaacaaaataaa

SEQ ID NO: 24 - DNA, 301 bp, synthetic construct
  misc_feature 1..301: note = Description of Artificial Sequence: Synthetic polynucleotide
  source 1..301: mol_type = other DNA
ccaatcgtggcatatcctctaaactttcttttcccttcataaatcctctttcttttttttccccctcacagttttcctgaacaggttgactattaattgtgtctgcttgatgtggacaccaggtggcgctggacatcagatttggagaggcagttgtctagggaaccgggctctgtgccagcgcaggaggcaggctggctctcctattccagggatgctcatccaggaaggaaaggttgcatgctggacacactaaccttgaagaattcttctgtctctctcgtcatttagaaaggaagga

SEQ ID NO: 25 - DNA, 305 bp, synthetic construct
  misc_feature 1..305: note = Description of Artificial Sequence: Synthetic polynucleotide
  source 1..305: mol_type = other DNA
ctagccaatcgtggcatatcctctaaactttcttttcccttcataaatcctctttcttttttttccccctcacagttttcctgaacaggttgactattaattgtgtctgcttgatgtggacaccaggtggcgctggacatcagatttggagaggcagttgtctagggaaccgggctctgtgccagcgcaggaggcaggctggctctcctactccagggatgctcatccaggaaggaaaggttgcatgctggacacactaaccttgaagaattcttctgtctctctcgtcatttagaaaggaagga

SEQ ID NO: 26 - DNA, 896 bp, synthetic construct
  misc_feature 1..896: note = Description of Artificial Sequence: Synthetic polynucleotide
  source 1..896: mol_type = other DNA
ctcgagttaattaatctcccacgccctggtctcagcttggggagtggtcagaccccaatggcgataaactctggcaactttatctgtgcactgcaggctcagccccaacagctttagctttcacaagcaggcaggggaagggaaacacatatctccagatatgaggttaattaacctgcaggctaaacccctcccccaccctagccccaagcttcatcttagctccactcctgaccctatccagctaaaggtccccacccagctcctgcctatctagtcattgcatatggcaagacttgaaagtcctatctcaaagcagcagaattatcagctacgactcctgcaggttataaccatcccccagcactccctgcccccacagcccagacttgaccaactcccagctccgcctgggacttccagatatggggccccacccttgcaggccttggggacgctgaagatattgactatctgcgtgccggaaaagggtgttataaaccggtaaaggctgggggtgggagtagcggatttgaagcacttgttggcctacagaggtgtggcaagcagagcacctcagaactcaggcgtactgcccgccgcccgagccctgcgagggccgatagcgagggtgtggcccttatctgcacccagcagagcgccggcggggtacggtcaccggtcccgggcagttgcctcagctgagtatgtcttctaaagataatgtcgattgtgtatggctgatgggattctaggaccaagcaagaggtttttttttttcccccacatacttaacgtttctatatttctatttgaattcgactggacagttccatttgaattatttctctctctctctctctctgacacattttatcttgccacccgggctcgag

SEQ ID NO: 27 - DNA, 320 bp, synthetic construct
  misc_feature 1..320: note = Description of Artificial Sequence: Synthetic polynucleotide
  source 1..320: mol_type = other DNA
ctggttctactcattacattccaatcgtggcatatcctctaaactttcttttcccttcataaatcctctttcttttttttccccctcacagttttcctgaacaggttgactattaattgtgtctgcttgatgtggacaccaggtggcgctggacatcagatttggagaggcagttgtctagggaaccgggctctgtgccagcgcaggaggcaggctggctctcctattccagggatgctcatccaggaaggaaaggttgcatgctggacacactaaccttgaagaattcttctgtctctctcgtcatttagaaaggaagg

SEQ ID NO: 28 - DNA, 300 bp, synthetic construct
  misc_feature 1..300: note = Description of Artificial Sequence: Synthetic polynucleotide
  source 1..300: mol_type = other DNA
ccaatcgtggcatatcctctaaactttcttttcccttcataaatcctctttcttttttttccccctcacagttttcctgaacaggttgactattaattgtgtctgcttgatgtggacaccaggtggcgctggacatcagatttggagaggcagttgtctagggaaccgggctctgtgccagcgcaggaggcaggctggctctcctattccagggatgctcatccaggaaggaaaggttgcatgctggacacactaaccttgaagaattcttctgtctctctcgtcatttagaaaggaagg

SEQ ID NO: 29 - DNA, 120 bp, synthetic construct
  misc_feature 1..120: note = Description of Artificial Sequence: Synthetic polynucleotide
  source 1..120: mol_type = other DNA
tcataaatcctctttcttttttttccccctcacagttttcctgaacaggttgactattaattgtgtctgcttgatgtggacaccaggtggcgctggacatcagatttggagaggcagttg

SEQ ID NO: 30 - DNA, 170 bp, synthetic construct
  misc_feature 1..170: note = Description of Artificial Sequence: Synthetic polynucleotide
  source 1..170: mol_type = other DNA
tcataaatcctctttcttttttttccccctcacagttttcctgaacaggttgactattaattgtgtctgcttgatgtggacaccaggtggcgctggacatcagatttggagaggcagttgtctagggaaccgggctctgtgccagcgcaggaggcaggctggctctccta

SEQ ID NO: 31 - DNA, 240 bp, synthetic construct
  misc_feature 1..240: note = Description of Artificial Sequence: Synthetic polynucleotide
  source 1..240: mol_type = other DNA
tcataaatcctctttcttttttttccccctcacagttttcctgaacaggttgactattaattgtgtctgcttgatgtggacaccaggtggcgctggacatcagatttggagaggcagttgtctagggaaccgggctctgtgccagcgcaggaggcaggctggctctcctattccagggatgctcatccaggaaggaaaggttgcatgctggacacactaaccttgaagaattcttctgtc

SEQ ID NO: 32 - DNA, 320 bp, synthetic construct; identical to SEQ ID NO: 27
  misc_feature 1..320: note = Description of Artificial Sequence: Synthetic polynucleotide
  source 1..320: mol_type = other DNA

SEQ ID NO: 33 - DNA, 816 bp, synthetic construct
  misc_feature 1..816: note = Description of Artificial Sequence: Synthetic polynucleotide
  source 1..816: mol_type = other DNA
tctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagcttatttaatcagagatgagatgctggaagggatagagggagctgagcttgtaaaaagtatagtaatcattcagcaaatggttttgaagcacctgctggatgctaaacactattttcagtgcttgaatcataaataagaacaaaacatgtatcttattccccacaagagtccaagtaaaaaataacagttaattataatgtgctctgtcccccaggctggagtgcagtggcacgatctcagctcactgcaacctccgcctcccgggttcaagcaattctcctgcctcagccaccctaatagctgggattacaggtgcacaccaccatgccaggctaatttttgtactttttgtagaggcagggtatcaccatgttgtccaagatggtcttgaactcctgagctccaagcagtccacccacctcagcctcccaaagtgct

SEQ ID NO: 34 - DNA, 1301 bp, synthetic construct
  misc_feature 1..1301: note = Description of Artificial Sequence: Synthetic polynucleotide
  source 1..1301: mol_type = other DNA
aagctttcatcaaaaaaagtctaaccagctgcattcgactttgactgcagcagctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctgagctcagtcttgtcatggcaaaacaaagataataatagtgtttttttatggagttagcgtgaggatggaaaacaatagcaaaattgattagactataaaaggtctcaacaaatagtagtagattttatcatccattaatccttccctctcctctcttactcatcccatcacgtatgcctcttaattttcccttacctataataagagttattcctcttattatattcttcttatagtgattctggatatcaaagtgggaatgaggggcaggccactaacgaagaagatgtttctcaaagaagccattctccccacatagatcatctcagcagggttcaggaagataaaggaggatcaaggtcgaaggtaggaactaaggaagaacactgggcaagtggatcc

SEQ ID NO: 35 - DNA, 754 bp, synthetic construct
  misc_feature 1..754: note = Description of Artificial Sequence: Synthetic polynucleotide
  source 1..754: mol_type = other DNA
taaatatatcatttaaatgcataaataagcaaaccctgctcgggaatgggagggagagtctctggagtccaccccttctcggccctggctctgcagatagtgctatcaaagccctgacagagccctgcccattgctgggccttggagtgagtcagcctagtagagaggcagggcaagccatctcatagctgctgagtgggagagagaaaagggctcattgtctataaactcaggtcatggctattcttattctcacactaagaaaaagaatgagatgtctacatataccctgcgtcccctcttgtgtactggggcccccaagagctctctaaaagtgatggcaaagtcattgcgctagatgccatcccatctattataaacctgcatttgtctccacacaccagtcatggacaataaccctcctcccaggtccacgtgcttgtctttgtataatactcaagtaatttcggaaaatgtattctttcaatcttgttctgttattcctgtttcaatggcttagtagaaaaagtacatacttgttttcccataaattgacaatagacaatttcacatcaatgtctatatgggtcgttgtgtttgctgtgtttgcaaaaactcacaataactttatattgttactactctaagaaagttacaacatggtgaatacaagagaaagctattacaagtccagaaaacaaaagttatcatcttgaggcctcagctttctaggaataatatcaatattacaaaa

SEQ ID NO: 36 - DNA, 1232 bp, synthetic construct
  misc_feature 1..1232: note = Description of Artificial Sequence: Synthetic polynucleotide
  source 1..1232: mol_type = other DNA
gcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttctccccttcctatgacatgaacttaaccatagaaaagaaggggaaagaaaacatcaagggtcccatagactcaccctgaagttctcaggatccacgtgcagcttgtcacagtgcagctcactcagctgggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacttcatccacgttcaccttgccccacagggcagtaacggcagacttctcctcaggagtcaggtgcaccatggtgtctgtttgaggttgctagtgaacacagttgtgtcagaagcaaatgt

SEQ ID NO: 37 - DNA, 12142 bp, synthetic construct
  misc_feature 1..12142: note = Description of Artificial Sequence: Synthetic polynucleotide
  source 1..12142: mol_type = other DNA
attggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagacgggtgcgagagcgtcagtattaagcgggggtgaataagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgttttaaaagaaaaggggggattggggggtacagtgcaggggaaagaatagtagacataatagcaacagacatacaaactaaagaattacaaaaacaaattacaaaaattcaaaattttatcggcgtgttgggggtggaccatcctctaggtattgaataagaaaaatgaagttaaggtggttgatggtaacactatgctaataactgcagagccagaagcaccataagggacatgataagggagccagcagacctctgatctcttcctgaatgctaatcttaaacatcctgaggaagaatgggacttccatttggggtgggcctatgatagggtaataagacagtagtgaatatcaagctacaaaaagccccctttcaaattcttctcagtcctaacttttcatactaagcccagtccttccaaagcagactgtgaaagagtgatagttccgggagactagcactgcagattccgggtcactgtgagtgggggaggcagggaagaagggctcacaggacagtcaaaccatgccccctgtttttccttcttcaagtagacctctataagacaacagagacaactaaggctgagtggccaggcgaggagaaaccatctcgccgtaaaacatggaaggaacacttcaggggaaaggtggtatctctaagcaagagaactgagtggagtcaaggctgagagatgcaggataagcaaatgggtagtgaaaagacattcatgaggacagctaaaacaataagtaatgtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttctccccttcctatgacatgaacttaaccatagaaaagaaggggaaagaaaacatcaagggtcccatagactcaccctgaagttctcaggatccacgtgcagcttgtcacagtgcagctcactcagctgggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacttcatccacgttcaccttgccccacagggcagtaacggcagacttctcctcaggagtcaggtgcaccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacgtaatatttggaatcacagcttggtaagcatattgaagatcgttttcccaattttcttattacacaaataagaaattgatgcactaaaagtggaagagttttgtctaccataattcagctttgggatatgtagatggatctcttcctgcgtctccagaatatgcaaaatacttacaggacagaatggatgaaaactctacctcagttctaagcatatcttctccttatttggattaaaaccttctggtaagaaaagaaaaaaaatatatatatatatgtgtatatatacacacatacatatacatatatatgcattcatttgttgttgtttttcttaatttgctcatggtatatgtgtatatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagcttatttaatcagagatgagatgctggaagggatagagggagctgagcttgtaaaaagtatagtaatcattcagcaaatggttttgaagcacctgctggatgctaaacactattttcagtgcttgaatcataaataagaataaaacatgtatcttattccccacaagagtccaagtaaaaaataacagttaattataatgtgctctgtcccccaggctggagtgcagtggcacgatctcagctcactgcaacctccgcctcccgggttcaagcaattctcctgcctcagccaccctaatagctgggattacaggtgcacaccaccatgccaggctaatttttgtactttttgtagaggcagggtatcaccatgttgtccaagatggtcttgaactcctgagctccaagcagtccacccacctcagcctcccaaagtgctatctgcggccgcctatctgtaccactagtctcgagaagctttcattaaaaaaagtctaaccagctgcattcgactttgactgcagcagctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctgagctcagtcttgtcatggcaaaataaagataataatagtgtttttttatggagttagcgtgaggatggaaaacaatagcaaaattgattagactataaaaggtctcaacaaatagtagtagattttatcatccattaatccttccctctcctctcttactcatcccatcacgtatgcctcttaattttcccttacctataataagagttattcctcttattatattcttcttatagtgattctggatattaaagtgggaatgaggggcaggccactaacgaagaagatgtttctcaaagaagccattctccccacatagatcatctcagcagggttcaggaagataaaggaggatcaaggtcgaaggtaggaactaaggaagaacactgggcaagtggatcctgagccccttttcctctaactgaaagaaggaaaaaaaaaatggaacccaaaatattctacatagtttccatgtcacagccagggctgggcagtctcctgttatttcttttaaaataaatatatcatttaaatgcataaataagcaaaccctgctcgggaatgggagggagagtctctggagtccaccccttctcggccctggctctgcagatagtgctatcaaagccctgacagagccctgcccattgctgggccttggagtgagtcagcctagtagagaggcagggcaagccatctcatagctgctgagtgggagagagaaaagggctcattgtctataaactcaggtcatggctattcttattctcacactaagaaaaagaatgagatgtctacatataccctgcgtcccctcttgtgtactggggcccccaagagctctctaaaagtgatggcaaagtcattgcgctagatgccatcccatctattataaacctgcatttgtctccacacaccagtcatggacaataaccctcctcccaggtccacgtgcttgtctttgtataatactcaagtaatttcggaaaatgtattctttcaatcttgttctgttattcctgtttcaatggcttagtagaaaaagtacatacttgttttcccataaattgacaatagacaatttcacatcaatgtctatatgggtcgttgtgtttgctgtgtttgcaaaaactcacaataactttatattgttactactctaagaaagttacaacatggtgaatacaagagaaagctattacaagtccagaaaataaaagttatcatcttgaggcctcagctttctaggaataatatcaatattacaaaatttaatctaacaattatgaacagcaatgagataatatgtacaaagtacccagacctatgtggtagagcatcaaggaagcgcattgcggagcagttttttgtttgtttgtttttgtattctgtttcgtgaggcaaggtttcactctgctgtccaggctggagtgcagtggcaagatcatgtctcactgcagccttgacacgcgtcgacggtaccgttaacgatcttagccactttttaaaagaaaaggggggactggaagggctaattcactcccaacgaagacaagatatcctgctagtccttcctttctaaatgacgagagagacagaagaattcttcaaggttagtgtgtccagcatgcaacctttccttcctggatgagcatccctggagtaggagagccagcctgcctcctgcgctggcacagagcccggttccctagacaactgcctctccaaatctgatgtccagcgccacctggtgtccacatcaagcagacacaattaatagtcaacctgttcaggaaaactgtgagggggaaaaaaaagaaagaggatttatgaagggaaaagaaagtttagaggatatgccacgattggctagcagctgctttttgcctgtactgggtctctctggttagaccagatctgagcctgggagctctctggctaactagggaacccactgcttaagcctcaataaagcttgccttgagtgcttcatccggaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtccggtagcttgccagcctcgactgtgccttctagttgccagccgtctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcacc

SEQ ID NO: 38 - DNA, 12040 bp, synthetic construct
  misc_feature 1..12040: note = Description of Artificial Sequence: Synthetic polynucleotide
  source 1..12040: mol_type = other DNA
attggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagacgggtgcgagagcgtcagtattaagcgggggtgaataagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgttttaaaagaaaaggggggattggggggtacagtgcaggggaaagaatagtagacataatagcaacagacatacaaactaaagaattacaaaaacaaattacaaaaattcaaaattttatcggcgtgttgggggtggaccatcctctaggtattgaataagaaaaatgaagttaaggtggttgatggtaacactatgctaataactgcagagccagaagcaccataagggacatgataagggagccagcagacctctgatctcttcctgaatgctaatcttaaacatcctgaggaagaatgggacttccatttggggtgggcctatgatagggtaataagacagtagtgaatatcaagctacaaaaagccccctttcaaattcttctcagtcctaacttttcatactaagcccagtccttccaaagcagactgtgaaagagtgatagttccgggagactagcactgcagattccgggtcactgtgagtgggggaggcagggaagaagggctcacaggacagtcaaaccatgccccctgtttttccttcttcaagtagacctctataagacaacagagacaactaaggctgagtggccaggcgaggagaaaccatctcgccgtaaaacatggaaggaacacttcaggggaaaggtggtatctctaagcaagagaactgagtggagtcaaggctgagagatgcaggataagcaaatgggtagtgaaaagacattcatgaggacagctaaaacaataagtaatgtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttctccccttcctatgacatgaacttaaccatagaaaagaaggggaaagaaaacatcaagggtcccatagactcaccctgaagttctcaggatccacgtgcagcttgtcacagtgcagctcactcagctgggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacttcatccacgttcaccttgccccacagggcagtaacggcagacttctcctcaggagtcaggtgcaccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacggcatctgtgaaggaaagaaacatctcctctaaaccactatgctgctagagcctcttttctgtactcaagcctcattcagacactagtgtcaccagtctcctcatatacctattgtattttcttcttcttgctggtttagtcatgttttctgggagcttaggggcttattttattttgttttgttttctaatcaacagagatgggcaaacccattatttttttctttagacttgggatggtgatagctgggcagcgtcagaaactgtgtgtggatatagataagagctcggactatgctgagctgtgatgagggagggacctagccaaaggcagtgagagtcagaatgctcctgctattgccttctcagtccccacgcttggtttctacacaagtagatacatagaaaaggctataggttagtgtttgagagtcctgcatgagttagttgctcagaaatgcccgataaatatgttatgtgtgtttatgtatatatatgttttatatatatatatgtgtgtgtgtgtgtgtgtgtgtgttgtgtttacaaatatgtgattatcatcaaaacgtgagggctaaagtgaccagataacttgcaggtctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagcttatttaatcagagatgagatgctggaagggatagagggagctgagcttgtaaaaagtatagtaatcattcagcaaatggttttgaagcacctgctggatgctaaacactattttcagtgcttgaatcataaataagaacaaaacatgtatcttattccccacaagagtccaagtaaaaaataacagttaattataatgtgctctgtcccccaggctggagtgcagtggcacgatctcagctcactgcaacctccgcctcccgggttcaagcaattctcctgcctcagccaccctaatagctgggattacaggtgcacaccaccatgccaggctaatttttgtactttttgtagaggcagggtatcaccatgttgtccaagatggtcttgaactcctgagctccaagcagtccacccacctcagcctcccaaagtgctatctgcggccgcctatctgtaccactagtctcgagaagctttcatcaaaaaaagtctaaccagctgcattcgactttgactgcagcagctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctgagctcagtcttgtcatggcaaaacaaagataataatagtgtttttttatggagttagcgtgaggatggaaaacaatagcaaaattgattagactataaaaggtctcaacaaatagtagtagattttatcatccattaatccttccctctcctctcttactcatcccatcacgtatgcctcttaattttcccttacctataataagagttattcctcttattatattcttcttatagtgattctggatatcaaagtgggaatgaggggcaggccactaacgaagaagatgtttctcaaagaagccattctccccacatagatcatctcagcagggttcaggaagataaaggaggatcaaggtcgaaggtaggaactaaggaagaacactgggcaagtggatcctaaatatatcatttaaatgcataaataagcaaaccctgctcgggaatgggagggagagtctctggagtccaccccttctcggccctggctctgcagatagtgctatcaaagccctgacagagccctgcccattgctgggccttggagtgagtcagcctagtagagaggcagggcaagccatctcatagctgctgagtgggagagagaaaagggctcattgtctataaactcaggtcatggctattcttattctcacactaagaaaaagaatgagatgtctacatataccctgcgtcccctcttgtgtactggggcccccaagagctctctaaaagtgatggcaaagtcattgcgctagatgccatcccatctattataaacctgcatttgtctccacacaccagtcatggacaataaccctcctcccaggtccacgtgcttgtctttgtataatactcaagtaatttcggaaaatgtattctttcaatcttgttctgttattcctgtttcaatggcttagtagaaaaagtacatacttgttttcccataaattgacaatagacaatttcacatcaatgtctatatgggtcgttgtgtttgctgtgtttgcaaaaactcacaataactttatattgttactactctaagaaagttacaacatggtgaatacaagagaaagctattacaagtccagaaaacaaaagttatcatcttgaggcctcagctttctaggaataatatcaatattacaaaacgcgtcgacggtaccgttaacgatcttagccactttttaaaagaaaaggggggactggaagggctaattcactcccaacgaagacaagatatcctgctagtccttcctttctaaatgacgagagagacagaagaattcttcaaggttagtgtgtccagcatgcaacctttccttcctggatgagcatccctggagtaggagagccagcctgcctcctgcgctggcacagagcccggttccctagacaactgcctctccaaatctgatgtccagcgccacctggtgtccacatcaagcagacacaattaatagtcaacctgttcaggaaaactgtgagggggaaaaaaaagaaagaggatttatgaagggaaaagaaagtttagaggatatgccacgattggctagcagctgctttttgcctgtactgggtctctctggttagaccagatctgagcctgggagctctctggctaactagggaacccactgcttaagcctcaataaagcttgccttgagtgcttcatccggaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtccggtagcttgccagcctcgactgtgccttctagttgccagccgtctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcacc

SEQ ID NO: 39 - DNA, 11438 bp, synthetic construct
  misc_feature 1..11438: note = Description of Artificial Sequence: Synthetic polynucleotide
  source 1..11438: mol_type = other DNA
attggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagacgggtgcgagagcgtcagtattaagcgggggtgaataagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgttttaaaagaaaaggggggattggggggtacagtgcaggggaaagaatagtagacataatagcaacagacatacaaactaaagaattacaaaaacaaattacaaaaattcaaaattttatcggcgtgttgggggtggaccatcctctaggtattgaataagaaaaatgaagttaaggtggttgatggtaacactatgctaataactgcagagccagaagcaccataagggacatgataagggagccagcagacctctgatctcttcctgaatgctaatcttaaacatcctgaggaagaatgggacttccatttggggtgggcctatgatagggtaataagacagtagtgaatatcaagctacaaaaagccccctttcaaattcttctcagtcctaacttttcatactaagcccagtccttccaaagcagactgtgaaagagtgatagttccgggagactagcactgcagattccgggtcactgtgagtgggggaggcagggaagaagggctcacaggacagtcaaaccatgccccctgtttttccttcttcaagtagacctctataagacaacagagacaactaaggctgagtggccaggcgaggagaaaccatctcgccgtaaaacatggaaggaacacttcaggggaaaggtggtatctctaagcaagagaactgagtggagtcaaggctgagagatgcaggataagcaaatgggtagtgaaaagacattcatgaggacagctaaaacaataagtaatgtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttctccccttcctatgacatgaacttaaccatagaaaagaaggggaaagaaaacatcaagggtcccatagactcaccctgaagttctcaggatccacgtgcagcttgtcacagtgcagctcactcagctgggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacttcatccacgttcaccttgccccacagggcagtaacggcagacttctcctcaggagtcaggtgcaccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttactctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagcttatttaatcagagatgagatgctggaagggatagagggagctgagcttgtaaaaagtatagtaatcattcagcaaatggttttgaagcacctgctggatgctaaacactattttcagtgcttgaatcataaataagaacaaaacatgtatcttattccccacaagagtccaagtaaaaaataacagttaattataatgtgctctgtcccccaggctggagtgcagtggcacgatctcagctcactgcaacctccgcctcccgggttcaagcaattctcctgcctcagccaccctaatagctgggattacaggtgcacaccaccatgccaggctaatttttgtactttttgtagaggcagggtatcaccatgttgtccaagatggtcttgaactcctgagctccaagcagtccacccacctcagcctcccaaagtgctatctgcggccgcctatctgtaccactagtctcgagaagctttcatcaaaaaaagtctaaccagctgcattcgactttgactgcagcagctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctgagctcagtcttgtcatggcaaaacaaagataataatagtgtttttttatggagttagcgtgaggatggaaaacaatagcaaaattgattagactataaaaggtctcaacaaatagtagtagattttatcatccattaatccttccctctcctctcttactcatcccatcacgtatgcctcttaattttcccttacctataataagagttattcctcttattatattcttcttatagtgattctggatatcaaagtgggaatgaggggcaggccactaacgaagaagatgtttctcaaagaagccattctccccacatagatcatctcagcagggttcaggaagataaaggaggatcaaggtcgaaggtaggaactaaggaagaacactgggcaagtggatcctaaatatatcatttaaatgcataaataagcaaaccctgctcgggaatgggagggagagtctctggagtccaccccttctcggccctggctctgcagatagtgctatcaaagccctgacagagccctgcccattgctgggccttggagtgagtcagcctagtagagaggcagggcaagccatctcatagctgctgagtgggagagagaaaagggctcattgtctataaactcaggtcatggctattcttattctcacactaagaaaaagaatgagatgtctacatataccctgcgtcccctcttgtgtactggggcccccaagagctctctaaaagtgatggcaaagtcattgcgctagatgccatcccatctattataaacctgcatttgtctccacacaccagtcatggacaataaccctcctcccaggtccacgtgcttgtctttgtataatactcaagtaatttcggaaaatgtattctttcaatcttgttctgttattcctgtttcaatggcttagtagaaaaagtacatacttgttttcccataaattgacaatagacaatttcacatcaatgtctatatgggtcgttgtgtttgctgtgtttgcaaaaactcacaataactttatattgttactactctaagaaagttacaacatggtgaatacaagagaaagctattacaagtccagaaaacaaaagttatcatcttgaggcctcagctttctaggaataatatcaatattacaaaacgcgtcgacggtaccgttaacgatcttagccactttttaaaagaaaaggggggactggaagggctaattcactcccaacgaagacaagatatcctgctagtccttcctttctaaatgacgagagagacagaagaattcttcaaggttagtgtgtccagcatgcaacctttccttcctggatgagcatccctggagtaggagagccagcctgcctcctgcgctggcacagagcccggttccctagacaactgcctctccaaatctgatgtccagcgccacctggtgtccacatcaagcagacacaattaatagtcaacctgttcaggaaaactgtgagggggaaaaaaaagaaagaggatttatgaagggaaaagaaagtttagaggatatgccacgattggctagcagctgctttttgcctgtactgggtctctctggttagaccagatctgagcctgggagctctctggctaactagggaacccactgcttaagcctcaataaagcttgccttgagtgcttcatccggaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtccggtagcttgccagcctcgactgtgccttctagttgccagccgtctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcacc

SEQ ID NO: 40 - DNA, 11342 bp, synthetic construct
  misc_feature 1..11342: note = Description of Artificial Sequence: Synthetic polynucleotide
  source 1..11342: mol_type = other DNA
attggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagacgggtgcgagagcgtcagtattaagcgggggtgaataagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgttttaaaagaaaaggggggattggggggtacagtgcaggggaaagaatagtagacataatagcaacagacatacaaactaaagaattacaaaaacaaattacaaaaattcaaaattttatcggcgtgttgggggtggaccatcctctaggtattgaataagaaaaatgaagttaaggtggttgatggtaacactatgctaataactgcagagccagaagcaccataagggacatgataagggagccagcagacctctgatctcttcctgaatgctaatcttaaacatcctgaggaagaatgggacttccatttggggtgggcctatgatagggtaataagacagtagtgaatatcaagctacaaaaagccccctttcaaattcttctcagtcctaacttttcatactaagcccagtccttccaaagcagactgtgaaagagtgatagttccgggagactagcactgcagattccgggtcactgtgagtgggggaggcagggaagaagggctcacaggacagtcaaaccatgccccctgtttttccttcttcaagtagacctctataagacaacagagacaactaaggctgagtggccaggcgaggagaaaccatctcgccgtaaaacatggaaggaacacttcaggggaaaggtggtatctctaagcaagagaactgagtggagtcaaggctgagagatgcaggataagcaaatgggtagtgaaaagacattcatgaggacagctaaaacaataagtaatgtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttctccccttcctatgacatgaacttaaccatagaaaagaaggggaaagaaaacatcaagggtcccatagactcaccctgaagttctcaggatccacgtgcagcttgtcacagtgcagctcactcagctgggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacttcatccacgttcaccttgccccacagggcagtaacggcagacttctcctcaggagtcaggtgcaccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacggcatctgtgaaggaaagaaacatctcctctaaaccactatgctgctagagcctcttttctgtactcaagcctcattcagacactagtgtcaccagtctcctcatatacctattgtattttcttcttcttgctggtttagtcatgttttctgggagcttaggggcttattttattttgttttgttttctaatcaacagagatgggcaaacccattatttttttctttagacttgggatggtgatagctgggcagcgtcagaaactgtgtgtggatatagataagagctcggactatgctgagctgtgatgagggagggacctagccaaaggcagtgagagtcagaatgctcctgctattgccttctcagtccccacgcttggtttctacacaagtagatacatagaaaaggctataggttagtgtttgagagtcctgcatgagttagttgctcagaaatgcccgataaatatgttatgtgtgtttatgtatatatatgttttatatatatatatgtgtgtgtgtgtgtgtgtgtgtgttgtgtttacaaatatgtgattatcatcaaaacgtgagggctaaagtgaccagataacttgcaggtctagacacccttttccggcacgcagatagtcaatatcttcagcgtccccaaggcctgcaagggtggggccccatatctggaagtcccaggcggagctgggagttggtcaagtctgggctgtgggggcagggagtgctgggggatggctcgagaagctttcatcaaaaaaagtctaaccagctgcattcgactttgactgcagcagctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctgagctcagtcttgtcatggcaaaacaaagataataatagtgtttttttatggagttagcgtgaggatggaaaacaatagcaaaattgattagactataaaaggtctcaacaaatagtagtagattttatcatccattaatccttccctctcctctcttactcatcccatcacgtatgcctcttaattttcccttacctataataagagttattcctcttattatattcttcttatagtgattctggatatcaaagtgggaatgaggggcaggccactaacgaagaagatgtttctcaaagaagccattctccccacatagatcatctcagcagggttcaggaagataaaggaggatcaaggtcgaaggtaggaactaaggaagaacactgggcaagtggatcctaaatatatcatttaaatgcataaataagcaaaccctgctcgggaatgggagggagagtctctggagtccaccccttctcggccctggctctgcagatagtgctatcaaagccctgacagagccctgcccattgctgggccttggagtgagtcagcctagtagagaggcagggcaagccatctcatagctgctgagtgggagagagaaaagggctcattgtctataaactcaggtcatggctattcttattctcacactaagaaaaagaatgagatgtctacatataccctgcgtcccctcttgtgtactggggcccccaagagctctctaaaagtgatggcaaagtcattgcgctagatgccatcccatctattataaacctgcatttgtctccacacaccagtcatggacaataaccctcctcccaggtccacgtgcttgtctttgtataatactcaagtaatttcggaaaatgtattctttcaatcttgttctgttattcctgtttcaatggcttagtagaaaaagtacatacttgttttcccataaattgacaatagacaatttcacatcaatgtctatatgggtcgttgtgtttgctgtgtttgcaaaaactcacaataactttatattgttactactctaagaaagttacaacatggtgaatacaagagaaagctattacaagtccagaaaacaaaagttatcatcttgaggcctcagctttctaggaataatatcaatattacaaaacgcgtcgacggtaccgttaacgatcttagccactttttaaaagaaaaggggggactggaagggctaattcactcccaacgaagacaagatatcctgctagtccttcctttctaaatgacgagagagacagaagaattcttcaaggttagtgtgtccagcatgcaacctttccttcctggatgagcatccctggagtaggagagccagcctgcctcctgcgctggcacagagcccggttccctagacaactgcctctccaaatctgatgtccagcgccacctggtgtccacatcaagcagacacaattaatagtcaacctgttcaggaaaactgtgagggggaaaaaaaagaaagaggatttatgaagggaaaagaaagtttagaggatatgccacgattggctagcagctgctttttgcctgtactgggtctctctggttagaccagatctgagcctgggagctctctggctaactagggaacccactgcttaagcctcaataaagcttgccttgagtgcttcatccggaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtccggtagcttgccagcctcgactgtgccttctagttgccagccgtctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcacc

SEQ ID NO: 41 - DNA, 30 bp, synthetic construct
  misc_feature 1..30: note = Description of Artificial Sequence: Synthetic probe
  source 1..30: mol_type = other DNA
acagccttctgatgtttctaacaggccagg

SEQ ID NO: 42 - DNA, 22 bp, synthetic construct
  misc_feature 1..22: note = Description of Artificial Sequence: Synthetic primer
  source 1..22: mol_type = other DNA
ggagctagaacgattcgcagtt

SEQ ID NO: 43 - DNA, 24 bp, synthetic construct
  misc_feature 1..24: note = Description of Artificial Sequence: Synthetic primer
  source 1..24: mol_type = other DNA
gttgtagctgtcccagtatttgtc

SEQ ID NO: 44 - DNA, 27 bp, synthetic construct
  misc_feature 1..27: note = Description of Artificial Sequence: Synthetic probe
  source 1..27: mol_type = other DNA
tgctgaaacattcaccttccatgcagt

SEQ ID NO: 45 - DNA, 25 bp, synthetic construct
  misc_feature 1..25: note = Description of Artificial Sequence: Synthetic primer
  source 1..25: mol_type = other DNA
tgaaacatacgttcccaaagagttt

SEQ ID NO: 46 - DNA, 26 bp, synthetic construct
  misc_feature 1..26: note = Description of Artificial Sequence: Synthetic primer
  source 1..26: mol_type = other DNA
ctctccttctcagaaagtgtgcatat

SEQ ID NO: 47 - DNA, 26 bp, synthetic construct
  misc_feature 1..26: note = Description of Artificial Sequence: Synthetic probe
  source 1..26: mol_type = other DNA
tacgagggctatgctctccctcacgc

SEQ ID NO: 48 - DNA, 19 bp, synthetic construct
  misc_feature 1..19: note = Description of Artificial Sequence: Synthetic primer
  source 1..19: mol_type = other DNA
tcacccacactgtgcccat

SEQ ID NO: 49 - DNA, 18 bp, synthetic construct
  misc_feature 1..18: note = Description of Artificial Sequence: Synthetic primer
  source 1..18: mol_type = other DNA
agccaggtccagacgcag

SEQ ID NO: 50 - DNA, 114 bp, synthetic construct
  misc_feature 1..114: note = Description of Artificial Sequence: Synthetic polynucleotide
  source 1..114: mol_type = other DNA
tgagccccttttcctctaactgaaagaaggaaaaaaaaaatggaacccaaaatattctacatagtttccatgtcacagccagggctgggcagtctcctgttatttcttttaaaa

SEQ ID NO: 51 - DNA, 197 bp, synthetic construct
  misc_feature 1..197: note = Description of Artificial Sequence: Synthetic polynucleotide
  source 1..197: mol_type = other DNA
tttaatctaacaattatgaacagcaatgagataatatgtacaaagtacccagacctatgtggtagagcatcaaggaagcgcattgcggagcagttttttgtttgtttgtttttgtattctgtttcgtgaggcaaggtttcactctgctgtccaggctggagtgcagtggcaagatcatgtctcactgcagccttgac

SEQ ID NO: 52 - DNA, 44 bp, synthetic construct
  misc_feature 1..44: note = Description of Artificial Sequence: Synthetic oligonucleotide
  source 1..44: mol_type = other DNA
gtatatgtgtatatatatatatatatattcaggaaataatatat

SEQ ID NO: 53 - DNA, 1232 bp, synthetic construct
  misc_feature 1..1232: note = Description of Artificial Sequence: Synthetic polynucleotide
  source 1..1232: mol_type = other DNA
gcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttctccccttcctatgacatgaacttaaccatagaaaagaaggggaaagaaaacatcaagggtcccatagactcaccctgaagttctcaggatccacgtgcagcttgtcacagtgcagctcactcagttgggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacttcatccacgttcaccttgccccacagggcagtaacggcagacttctcctcaggagtcaggtgcaccatggtgtctgtttgaggttgctagtgaacacagttgtgtcagaagcaaatgt

SEQ ID NO: 54 - DNA, 1232 bp, synthetic construct
  misc_feature 1..1232: note = Description of Artificial Sequence: Synthetic polynucleotide
  source 1..1232: mol_type = other DNA
gcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttctccccttcctatgacatgaacttaaccatagaaaagaaggggaaagaaaacatcaagggtcccatagactcaccctgaagttctcaggatccacgtgcagcttgtcacagtgcagctcactcagtgtggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacggcatccacgttcaccttgccccacagggcagtaacggcagacttctcctcaggagtcaggtgcaccatggtgtctgtttgaggttgctagtgaacacagttgtgtcagaagcaaatgt

SEQ ID NO: 55 - DNA, 1232 bp, synthetic construct
  misc_feature 1..1232: note = Description of Artificial Sequence: Synthetic polynucleotide
  source 1..1232: mol_type = other DNA
gcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttctccccttcctatgacatgaacttaaccatagaaaagaaggggaaagaaaacatcaagggtcccatagactcaccctgaagttctcaggatccacgtgcagcttgtcacagtgcagctcactcagtgtggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaactgcatccacgttcaccttgccccacagggcagtaacggcagacttctcctcaggagtcaggtgcaccatggtgtctgtttgaggttgctagtgaacacagttgtgtcagaagcaaatgt

SEQ ID NO: 56 - DNA, 1232 bp, synthetic construct
  misc_feature 1..1232: note = Description of Artificial Sequence: Synthetic polynucleotide
  source 1..1232: mol_type = other DNA
gcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttctccccttcctatgacatgaacttaaccatagaaaagaaggggaaagaaaacatcaagggtcccatagactcaccctgaagttctcaggatccacgtgcagcttgtcacagtgcagctcactcagtgtggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacagcatccacgttcaccttgccccacagggcagtaacggcagacttctcctcaggagtcaggtgcaccatggtgtctgtttgaggttgctagtgaacacagttgtgtcagaagcaaatgt

SEQ ID NO: 57 - DNA, 1232 bp, synthetic construct
  misc_feature 1..1232: note = Description of Artificial Sequence: Synthetic polynucleotide
  source 1..1232: mol_type = other DNA
gcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttctccccttcctatgacatgaacttaaccatagaaaagaaggggaaagaaaacatcaagggtcccatagactcaccctgaagttctcaggatccacgtgcagcttgtcacagtgcagctcactcagtgtggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaaccgcatccacgttcaccttgccccacagggcagtaacggcagacttctcctcaggagtcaggtgcaccatggtgtctgtttgaggttgctagtgaacacagttgtgtcagaagcaaatgt

SEQ ID NO: 58 - DNA, 1232 bp, synthetic construct
  misc_feature 1..1232: note = Description of Artificial Sequence: Synthetic polynucleotide
  source 1..1232: mol_type = other DNA
gcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttctccccttcctatgacatgaacttaaccatagaaaagaaggggaaagaaaacatcaagggtcccatagactcaccctgaagttctcaggatccacgtgcagcttgtcacagtgcagctcactcagtgtggcaaaggtgcccttgagcttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacttcatccacgttcaccttgccccacagggcagtaacggcagacttctcctcaggagtcaggtgcaccatggtgtctgtttgaggttgctagtgaacacagttgtgtcagaagcaaatgt

SEQ ID NO: 59 - DNA, 1232 bp, synthetic construct
  misc_feature 1..1232: note = Description of Artificial Sequence: Synthetic polynucleotide
  source 1..1232: mol_type = other DNA
gcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttctccccttcctatgacatgaacttaaccatagaaaagaaggggaaagaaaacatcaagggtcccatagactcaccctgaagttctcaggatccacgtgcagcttgtcacagtgcagctcactcagtgtggcaaaggtgcccttgagtttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacttcatccacgttcaccttgccccacagggcagtaacggcagacttctcctcaggagtcaggtgcaccatggtgtctgtttgaggttgctagtgaacacagttgtgtcagaagcaaatgt

SEQ ID NO: 60 - DNA, 1232 bp, synthetic construct
  misc_feature 1..1232: note = Description of Artificial Sequence: Synthetic polynucleotide
  source 1..1232: mol_type = other DNA
gcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttctccccttcctatgacatgaacttaaccatagaaaagaaggggaaagaaaacatcaagggtcccatagactcaccctgaagttctcaggatccacgtgcagcttgtcacagtgcagctcactcagtgtggcaaaggtgcccttgagcttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacggcatccacgttcaccttgccccacagggcagtaacggcagacttctcctcaggagtcaggtgcaccatggtgtctgtttgaggttgctagtgaacacagttgtgtcagaagcaaatgt

SEQ ID NO: 61 - DNA, 1232 bp, synthetic construct
  misc_feature 1..1232: note = Description of Artificial Sequence: Synthetic polynucleotide
  source 1..1232: mol_type = other DNA
gcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttctccccttcctatgacatgaacttaaccatagaaaagaaggggaaagaaaacatcaagggtcccatagactcaccctgaagttctcaggatccacgtgcagcttgtcacagtgcagctcactcagtgtggcaaaggtgcccttgagtttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacggcatccacgttcaccttgccccacagggcagtaacggcagacttctcctcaggagtcaggtgcaccatggtgtctgtttgaggttgctagtgaacacagttgtgtcagaagcaaatgt

SEQ ID NO: 62 - DNA, 1232 bp, synthetic construct
  misc_feature 1..1232: note = Description of Artificial Sequence: Synthetic polynucleotide
  source 1..1232: mol_type = other DNA
gcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttctccccttcctatgacatgaacttaaccatagaaaagaaggggaaagaaaacatcaagggtcccatagactcaccctgaagttctcaggatccacgtgcagcttgtcacagtgcagctcactcagtgtggcaaaggtgcccttgagcttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaactgcatccacgttcaccttgccccacagggcagtaacggcagacttctcctcaggagtcaggtgcaccatggtgtctgtttgaggttgctagtgaacacagttgtgtcagaagcaaatgt

SEQ ID NO: 63 - DNA, 1232 bp, synthetic construct
  misc_feature 1..1232: note = Description of Artificial Sequence: Synthetic polynucleotide
  source 1..1232: mol_type = other DNA
gcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttctccccttcctatgacatgaacttaaccatagaaaagaaggggaaagaaaacatcaagggtcccatagactcaccctgaagttctcaggatccacgtgcagcttgtcacagtgcagctcactcagtgtggcaaaggtgcccttgagcttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacagcatccacgttcaccttgccccacagggcagtaacggcagacttctcctcaggagtcaggtgcaccatggtgtctgtttgaggttgctagtgaacacagttgtgtcagaagcaaatgt

SEQ ID NO: 64 - DNA, 1232 bp, synthetic construct
  misc_feature 1..1232: note = Description of Artificial Sequence: Synthetic polynucleotide
  source 1..1232: mol_type = other DNA
gcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttctccccttcctatgacatgaacttaaccatagaaaagaaggggaaagaaaacatcaagggtcccatagactcaccctgaagttctcaggatccacgtgcagcttgtcacagtgcagctcactcagtgtggcaaaggtgcccttgagcttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaaccgcatccacgttcaccttgccccacagggcagtaacggcagacttctcctcaggagtcaggtgcaccatggtgtctgtttgaggttgctagtgaacacagttgtgtcagaagcaaatgt

SEQ ID NO: 65 - DNA, 1232 bp, synthetic construct
  misc_feature 1..1232: note = Description of Artificial Sequence: Synthetic polynucleotide
  source 1..1232: mol_type = other DNA
gcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttctccccttcctatgacatgaacttaaccatagaaaagaaggggaaagaaaacatcaagggtcccatagactcaccctgaagttctcaggatccacgtgcagcttgtcacagtgcagctcactcagtgtggcaaaggtgcccttgagtttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaactgcatccacgttcaccttgccccacagggcagtaacggcagacttctcctcaggagtcaggtgcaccatggtgtctgtttgaggttgctagtgaacacagttgtgtcagaagcaaatgt

SEQ ID NO: 66 - DNA, 1232 bp, synthetic construct
  misc_feature 1..1232: note = Description of Artificial Sequence: Synthetic polynucleotide
  source 1..1232: mol_type = other DNA
gcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttctccccttcctatgacatgaacttaaccatagaaaagaaggggaaagaaaacatcaagggtcccatagactcaccctgaagttctcaggatccacgtgcagcttgtcacagtgcagctcactcagtgtggcaaaggtgcccttgagtttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacagcatccacgttcaccttgccccacagggcagtaacggcagacttctcctcaggagtcaggtgcaccatggtgtctgtttgaggttgctagtgaacacagttgtgtcagaagcaaatgt

SEQ ID NO: 67 - DNA, 1232 bp, synthetic construct
  misc_feature 1..1232: note = Description of Artificial Sequence: Synthetic polynucleotide
  source 1..1232: mol_type = other DNA
gcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttctccccttcctatgacatgaacttaaccatagaaaagaaggggaaagaaaacatcaagggtcccatagactcaccctgaagttctcaggatccacgtgcagcttgtcacagtgcagctcactcagtgtggcaaaggtgcccttgagtttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaaccgcatccacgttcaccttgccccacagggcagtaacggcagacttctcctcaggagtcaggtgcaccatggtgtctgtttgaggttgctagtgaacacagttgtgtcagaagcaaatgt

SEQ ID NO: 68 - DNA, 1232 bp, synthetic construct
  misc_feature 1..1232: note = Description of Artificial Sequence: Synthetic polynucleotide
  source 1..1232: mol_type = other DNA
gcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttctccccttcctatgacatgaacttaaccatagaaaagaaggggaaagaaaacatcaagggtcccatagactcaccctgaagttctcaggatccacgtgcagcttgtcacagtgcagctcactcagctgggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacggcatccacgttcaccttgccccacagggcagtaacggcagacttctcctcaggagtcaggtgcaccatggtgtctgtttgaggttgctagtgaacacagttgtgtcagaagcaaatgt

SEQ ID NO: 69 - DNA, 1232 bp, synthetic construct
  misc_feature 1..1232: note = Description of Artificial Sequence: Synthetic polynucleotide
  source 1..1232: mol_type = other DNA
gcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttctccccttcctatgacatgaacttaaccatagaaaagaaggggaaagaaaacatcaagggtcccatagactcaccctgaagttctcaggatccacgtgcagcttgtcacagtgcagctcactcagttgggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacggcatccacgttcaccttgccccacagggcagtaacggcagacttctcctcaggagtcaggtgcaccatggtgtctgtttgaggttgctagtgaacacagttgtgtcagaagcaaatgt

SEQ ID NO: 70 - DNA, 1232 bp, synthetic construct
  misc_feature 1..1232: note = Description of Artificial Sequence: Synthetic polynucleotide
  source 1..1232: mol_type = other DNA
gcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttctccccttcctatgacatgaacttaaccatagaaaagaaggggaaagaaaacatcaagggtcccatagactcaccctgaagttctcaggatccacgtgcagcttgtcacagtgcagctcactcagctgggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaactgcatccacgttcaccttgccccacagggcagtaacggcagacttctcctcaggagtcaggtgcaccatggtgtctgtttgaggttgctagtgaacacagttgtgtcagaagcaaatgt

SEQ ID NO: 71 - DNA, 1232 bp, synthetic construct
  misc_feature 1..1232: note = Description of Artificial Sequence: Synthetic polynucleotide
  source 1..1232: mol_type = other DNA
gcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttctccccttcctatgacatgaacttaaccatagaaaagaaggggaaagaaaacatcaagggtcccatagactcaccctgaagttctcaggatccacgtgcagcttgtcacagtgcagctcactcagctgggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacagcatccacgttcaccttgccccacagggcagtaacggcagacttctcctcaggagtcaggtgcaccatggtgtctgtttgaggttgctagtgaacacagttgtgtcagaagcaaatgt

SEQ ID NO: 72 - DNA, 1232 bp, synthetic construct
  misc_feature 1..1232: note = Description of Artificial Sequence: Synthetic polynucleotide
  source 1..1232: mol_type = other DNA
gcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttctccccttcctatgacatgaacttaaccatagaaaagaaggggaaagaaaacatcaagggtcccatagactcaccctgaagttctcaggatccacgtgcagcttgtcacagtgcagctcactcagctgggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaaccgcatccacgttcaccttgccccacagggcagtaacggcagacttctcctcaggagtcaggtgcaccatggtgtctgtttgaggttgctagtgaacacagttgtgtcagaagcaaatgt

SEQ ID NO: 73 - DNA, 1232 bp, synthetic construct
  misc_feature 1..1232: note = Description of Artificial Sequence: Synthetic polynucleotide
  source 1..1232: mol_type = other DNA
gcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttctccccttcctatgacatgaacttaaccatagaaaagaaggggaaagaaaacatcaagggtcccatagactcaccctgaagttctcaggatccacgtgcagcttgtcacagtgcagctcactcagttgggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaactgcatccacgttcaccttgccccacagggcagtaacggcagacttctcctcaggagtcaggtgcaccatggtgtctgtttgaggttgctagtgaacacagttgtgtcagaagcaaatgt

SEQ ID NO: 74 - DNA, 1232 bp, synthetic construct
  misc_feature 1..1232: note = Description of Artificial Sequence: Synthetic polynucleotide
  source 1..1232: mol_type = other DNA
gcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttctccccttcctatgacatgaacttaaccatagaaaagaaggggaaagaaaacatcaagggtcccatagactcaccctgaagttctcaggatccacgtgcagcttgtcacagtgcagctcactcagttgggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacagcatccacgttcaccttgccccacagggcagtaacggcagacttctcctcaggagtcaggtgcaccatggtgtctgtttgaggttgctagtgaacacagttgtgtcagaagcaaatgt

SEQ ID NO: 75 - DNA, 1232 bp, synthetic construct
  misc_feature 1..1232: note = Description of Artificial Sequence: Synthetic polynucleotide
  source 1..1232: mol_type = other DNA
gcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttctccccttcctatgacatgaacttaaccatagaaaagaaggggaaagaaaacatcaagggtcccatagactcaccctgaagttctcaggatccacgtgcagcttgtcacagtgcagctcactcagttgggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaaccgcatccacgttcaccttgccccacagggcagtaacggcagacttctcctcaggagtcaggtgcaccatggtgtctgtttgaggttgctagtgaacacagttgtgtcagaagcaaatgt

SEQ ID NO: 76 - DNA, 8674 bp, synthetic construct
  misc_feature 1..8674: note = Description of Artificial Sequence: Synthetic polynucleotide
  source 1..8674: mol_type = other DNA
attggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagacgggtgcgagagcgtcagtattaagcgggggtgaataagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgttttaaaagaaaaggggggattggggggtacagtgcaggggaaagaatagtagacataatagcaacagacatacaaactaaagaattacaaaaacaaattacaaaaattcaaaattttatcggcgtgttgggggtggaccatcctctaggtattgaataagaaaaatgaagttaaggtggttgatggtaacactatgctaataactgcagagccagaagcaccataagggacatgataagggagccagcagacctctgatctcttcctgaatgctaatcttaaacatcctgaggaagaatgggacttccatttggggtgggcctatgatagggtaataagacagtagtgaatatcaagctacaaaaagccccctttcaaattcttctcagtcctaacttttcatactaagcccagtccttccaaagcagactgtgaaagagtgatagttccgggagactagcactgcagattccgggtcactgtgagtgggggaggcagggaagaagggctcacaggacagtcaaaccatgccccctgtttttccttcttcaagtagacctctataagacaacagagacaactaaggctgagtggccaggcgaggagaaaccatctcgccgtaaaacatggaaggaacacttcaggggaaaggtggtatctctaagcaagagaactgagtggagtcaaggctgagagatgcaggataagcaaatgggtagtgaaaagacattcatgaggacagctaaaacaataagtaatgtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttctccccttcctatgacatgaacttaaccatagaaaagaaggggaaagaaaacatcaagggtcccatagactcaccctgaagttctcaggatccacgtgcagcttgtcacagtgcagctcactcagctgggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacttcatccacgttcaccttgccccacagggcagtaacggcagacttctcctcaggagtcaggtgcaccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttaccacccttttccggcacgcagatagtcaatatcttcagcgtccccaaggcctgcaagggtggggccccatatctggaagtcccaggcggagctgggagttggtcaagtctgggctgtgggggcagggagtgctgggggatggacgcgtcgacggtaccgttaacgatcttagccactttttaaaagaaaaggggggactggaagggctaattcactcccaacgaagacaagatatcctgctagtccttcctttctaaatgacgagagagacagaagaattcttcaaggttagtgtgtccagcatgcaacctttccttcctggatgagcatccctggagtaggagagccagcctgcctcctgcgctggcacagagcccggttccctagacaactgcctctccaaatctgatgtccagcgccacctggtgtccacatcaagcagacacaattaatagtcaacctgttcaggaaaactgtgagggggaaaaaaaagaaagaggatttatgaagggaaaagaaagtttagaggatatgccacgattggctagcagctgctttttgcctgtactgggtctctctggttagaccagatctgagcctgggagctctctggctaactagggaacccactgcttaagcctcaataaagcttgccttgagtgcttcatccggaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtccggtagcttgccagcctcgactgtgccttctagttgccagccgtctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcacc

SEQ ID NO: 77 - DNA, 10011 bp, synthetic construct
  misc_feature 1..10011: note = Description of Artificial Sequence: Synthetic polynucleotide
  source 1..10011: mol_type = other DNA
attggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagacgggtgcgagagcgtcagtattaagcgggggtgaataagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgttttaaaagaaaaggggggattggggggtacagtgcaggggaaagaatagtagacataatagcaacagacatacaaactaaagaattacaaaaacaaattacaaaaattcaaaattttatcggcgtgttgggggtggaccatcctctaggtattgaataagaaaaatgaagttaaggtggttgatggtaacactatgctaataactgcagagccagaagcaccataagggacatgataagggagccagcagacctctgatctcttcctgaatgctaatcttaaacatcctgaggaagaatgggacttccatttggggtgggcctatgatagggtaataagacagtagtgaatatcaagctacaaaaagccccctttcaaattcttctcagtcctaacttttcatactaagcccagtccttccaaagcagactgtgaaagagtgatagttccgggagactagcactgcagattccgggtcactgtgagtgggggaggcagggaagaagggctcacaggacagtcaaaccatgccccctgtttttccttcttcaagtagacctctataagacaacagagacaactaaggctgagtggccaggcgaggagaaaccatctcgccgtaaaacatggaaggaacacttcaggggaaaggtggtatctctaagcaagagaactgagtggagtcaaggctgagagatgcaggataagcaaatgggtagtgaaaagacattcatgaggacagctaaaacaataagtaatgtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttctccccttcctatgacatgaacttaaccatagaaaagaaggggaaagaaaacatcaagggtcccatagactcaccctgaagttctcaggatccacgtgcagcttgtcacagtgcagctcactcagctgggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacttcatccacgttcaccttgccccacagggcagtaacggcagacttctcctcaggagtcaggtgcaccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttaccacccttttccggcacgcagatagtcaatatcttcagcgtccccaaggcctgcaagggtggggccccatatctggaagtcccaggcggagctgggagttggtcaagtctgggctgtgggggcagggagtgctgggggatggacgcgtatctgcggccgcctatctgtaccactagtctcgagaagctttcatcaaaaaaagtctaaccagctgcattcgactttgactgcagcagctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctgagctcagtcttgtcatggcaaaacaaagataataatagtgtttttttatggagttagcgtgaggatggaaaacaatagcaaaattgattagactataaaaggtctcaacaaatagtagtagattttatcatccattaatccttccctctcctctcttactcatcccatcacgtatgcctcttaattttcccttacctataataagagttattcctcttattatattcttcttatagtgattctggatatcaaagtgggaatgaggggcaggccactaacgaagaagatgtttctcaaagaagccattctccccacatagatcatctcagcagggttcaggaagataaaggaggatcaaggtcgaaggtaggaactaaggaagaacactgggcaagtgacgcgtcgacggtaccgttaacgatcttagccactttttaaaagaaaaggggggactggaagggctaattcactcccaacgaagacaagatatcctgctagtccttcctttctaaatgacgagagagacagaagaattcttcaaggttagtgtgtccagcatgcaacctttccttcctggatgagcatccctggagtaggagagccagcctgcctcctgcgctggcacagagcccggttccctagacaactgcctctccaaatctgatgtccagcgccacctggtgtccacatcaagcagacacaattaatagtcaacctgttcaggaaaactgtgagggggaaaaaaaagaaagaggatttatgaagggaaaagaaagtttagaggatatgccacgattggctagcagctgctttttgcctgtactgggtctctctggttagaccagatctgagcctgggagctctctggctaactagggaacccactgcttaagcctcaataaagcttgccttgagtgcttcatccggaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtccggtagcttgccagcctcgactgtgccttctagttgccagccgtctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcacc

SEQ ID NO: 78 - DNA, 879 bp, Homo sapiens
  source 1..879: mol_type = genomic DNA
ctaggtattgaataagaaaaatgaagttaaggtggttgatggtaacactatgctaataactgcagagccagaagcaccataagggacatgataagggagccagcagacctctgatctcttcctgaatgctaatcttaaacatcctgaggaagaatgggacttccatttggggtgggcctatgatagggtaataagacagtagtgaatatcaagctacaaaaagccccctttcaaattcttctcagtcctaacttttcatactaagcccagtccttccaaagcagactgtgaaagagtgatagttccgggagactagcactgcagattccgggtcactgtgagtgggggaggcagggaagaagggctcacaggacagtcaaaccatgccccctgtttttccttcttcaagtagacctctataagacaacagagacaactaaggctgagtggccaggcgaggagaaaccatctcgccgtaaaacatggaaggaacacttcaggggaaaggtggtatctctaagcaagagaactgagtggagtcaaggctgagagatgcaggataagcaaatgggtagtgaaaagacattcatgaggacagctaaaacaataagtaatgtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcatt

(19) United States
(12) Patent Application Publication
(10) Pub. No.: US 2024/0287543 A1
(43) Pub. Date: Aug. 29, 2024

(54) GLOBIN GENE THERAPY FOR TREATING HEMOGLOBINOPATHIES

(71) Applicant: MEMORIAL SLOAN-KETTERING CANCER CENTER, New York, NY (US)

(72) Inventors: Michel Sadelain, New York, NY (US); Annalisa Cabriolu, Villacidro (CA) (IT)

(73) Assignee: MEMORIAL SLOAN-KETTERING CANCER CENTER, New York, NY (US)

(21) Appl. No.: 18/355,575

(22) Filed: Jul. 20, 2023

Related U.S. Application Data
(63) Continuation of application No. 16/890,436, filed on Jun. 2, 2020, now Pat. No. 11,753,654.
Application No. 16/890,436 is a continuation of application No. PCT/US2018/064256, filed on Dec. 6, 2018.
(60) Provisional application No. 62/595,277, filed on Dec. 6, 2017.

(51) Int. Cl. C12N 15/86; A61P 7/00; C12N 9/22

(52) U.S. Cl. CPC C12N 15/86; A61P 7/00; C12N 9/22; C12N 2310/20; C12N 2830/008; C12N 2830/15; C12N 2830/30

(57) ABSTRACT

The presently disclosed subject matter provides for expression cassettes that allow for expression of a globin gene or a functional portion thereof, vectors comprising thereof, and cells transduced with such expression cassettes and vectors. The presently disclosed subject matter further provides methods for treating a hemoglobinopathy in a subject comprising administering an effective amount of such transduced cells to the subject.

### CROSS-REFERENCE TO RELATED APPLICATIONS

This application is a continuation of U.S. patent application Ser. No. 16/890,436, filed Jun. 2, 2020, which is a continuation of International Patent Application No. PCT/US18/064256 filed on Dec. 6, 2018, which claims priority to U.S. Provisional Application No. 62/595,277 filed on Dec. 6, 2017, the content of each of which is incorporated by reference in its entirety, and to each of which priority is claimed.

### SEQUENCE LISTING

The specification further incorporates by reference the Sequence Listing submitted on Jul. 20, 2023. The Sequence Listing file, identified as 0727341469.xml, is 293,034 bytes in size and was created on Jul. 20, 2023. The Sequence Listing, electronically filed on Jul. 20, 2023, does not extend beyond the scope of the specification and thus does not contain new matter.

### INTRODUCTION

The presently disclosed subject matter provides expression cassettes and vectors comprising such expression cassettes that express a globin protein, e.g., a human β-globin protein. The presently disclosed subject matter further provides expression cassettes that comprise a globin gene or a functional portion thereof operably linked to a β-globin locus control region (LCR) comprising a plurality of Dnase I hypersensitive sites. The expression cassettes of the presently disclosed subject matter comprise one or more insulators that counteract the effect of enhancer elements. The insulators disclosed herein do not substantially adversely impact the titer of a vector that comprises the presently disclosed expression cassettes. The expression cassettes and vectors can be used for treating a hemoglobinopathy, e.g., β-thalassemia, and sickle cell anemia.

### BACKGROUND

β-thalassemia and sickle cell anemia are severe congenital anemias that are caused by defective production of the β chain of hemoglobin. In β-thalassemia, the β chain deficit leads to the intracellular precipitation of excess α-globin chains, causing ineffective erythropoiesis and hemolytic anemia (Weatherall and Clegg (1981), Stamatoyannopoulos et al., (1994), Weatherall (2001), Steinberg (2001)). In the most severe forms found in homozygotes or compound heterozygotes, anemia is lethal within the first years of life in the absence of any treatment (Cooley and Lee (1925)). Lifelong transfusion therapy is needed to correct anemia, suppress ineffective erythropoiesis and inhibit gastrointestinal iron absorption (Weatherall and Clegg (1981), Stamatoyannopoulos et al. (1994), Weatherall (2001), Steinberg (2001)). However, transfusion therapy itself leads to iron overload, which is lethal if untreated. The prevention and treatment of iron overload are the major goals of current patient management (Giardina (2001)). The only current curative treatment to cure β-thalassemia is to provide erythroid precursors harboring normal globin genes through allogeneic bone marrow transplantation (BMT) (Giardini and Lucarelli (1994), Boulad et al. (1998), Lucarelli et al. (1999), Tisdale and Sadelain (2001)).

In sickle cell anemia, the hemoglobin β chain is mutated at amino acid position 6 (Glu→Val), leading to the synthesis of BS instead of the normal BA chain (Steinberg (2001), Pauling et al. (1949)). The resulting hemoglobin, HbS, causes accelerated red cell destruction, erythroid hyperplasia and painful vaso-occlusive ‘crises’ (Steinberg (2001)). Vaso-occlusion can damage organs, eventually causing long-term disabilities (e.g. following stroke or bone necrosis), and sometimes sudden death. While a very serious disorder, the course of sickle cell disease is typically unpredictable (Steinberg (2001)). By increasing production of fetal hemoglobin (Swank and Stamatoyannopoulos (1998)) and suppressing hematopoiesis, hydroxyurea can produce a measurable clinical benefit (Platt et al. (1984)), Charache et al. (1992), Atweh and Loukopoulos (2001)). Since hydroxyurea is a cytotoxic agent, there is a great need for alternative, less toxic drugs to induce γ-globin gene expression (Perrine et al. (2005), Stamatoyannopoulos (2005)). As for β-thalassemia, allogeneic bone marrow transplantation (BMT) is at present the only curative therapy for sickle cell disease (Tisdale and Sadelain (2001), Vermylen et al. (1998), Luzzatto and Goodfellow (1989)).

BMT, however, is not available as a therapeutic option to most patients suffering from β-thalassemia or sickle cell disease, due to the lack of an HLA-matched bone marrow donor for most individuals. Furthermore, although potentially curative, allogeneic BMT is not devoid of complications. Safe transplantation requires the identification of a histo-compatible donor to minimize the risks of graft rejection and graft-versus-host disease (Tisdale and Sadelain (2001), Vermylen et al. (1998), Luzzatto and Goodfellow (1989)). Because of the greater risks associated with matched-unrelated or mismatched transplants, most patients have to settle for life-long transfusion therapy, which does not correct ineffective erythropoiesis and exacerbates systemic iron accumulation. Moreover, despite the considerable improvement in life expectancy in the last decades (Borgna-Pignatti et al. (2004), Telfer et al. (2009), Ladis et al. (2011)), the risk of some serious complications arising over the long term from viral infections, iron toxicity and liver cirrhosis, remain (Mancuso et al. (2006)). These medical risks, together with the socio-economic cost of chronic β-thalassemia, underscore the need for safe, effective and curative therapies.

The only means to cure rather than treat severe β-thalassemia is to provide the patient with healthy hematopoietic stem cells (HSCs). HSCs normally give rise to all blood cell types, including 20 billion RBCs per day in adults. HSCs can be harvested from a donor with wild-type β-globin genes to yield long-lived red blood cells (RBCs) with a normal content in hemoglobin. Alternatively, one may genetically correct the patient's own HSCs, which at once resolves the search for a donor and eliminates the risks of graft-versus-host disease and graft rejection associated with allogeneic BMT (Sadelain (1997), Sadelain et al. (2007)). Globin gene transfer aims to restore the capacity of the β-thalassemic subject's own blood-forming stem cells to generate RBCs with a sufficient hemoglobin content Sadelain et al. (2007), Persons and Tisdale (2004), Sadelain (2006)). The goal in patients with sickle cell anemia is to prevent sickling, which can be achieved by diluting the endogenous HbS with a non-sickling Hb that incorporates the vector-encoded globin chain. The patient's own HSCs are the cells that have to be genetically modified to ensure long-lasting therapeutic benefits and achieve a curative stem cell-based therapy.

The implementation of globin gene transfer for the treatment of severe β-thalassemia and sickle cell anemia requires the efficient introduction of a regulated human β- or β-like globin gene in HSCs. The β-globin gene (or β-like variant) must be expressed in erythroid-specific fashion and at high level, especially for the treatment of transfusion-dependent beta-zero thalassemias.

The globin vectors developed to date present shortcomings that may limit or even preclude their safe use in thalassemia and sickle cell patients. Some of the β-globin locus control region (LCR) components contained in the vectors, in particular Dnase I hypersensitive site-2 (HS2), may have non-erythroid activity, exposing patients to the risk of insertional oncogenesis as seen with non-specific expression vectors. Further, the use of large LCR segments can be detrimental to the production of high titer vectors and the efficient transduction of patients HSCs. Accordingly, there is a need for novel globin expression cassettes that allow for therapeutic expression of a globin gene (e.g., human β-globin gene) in erythroid-specific and differentiation stage-specific fashion with minimal risk of insertional oncogenesis, and that enable high level transduction, thus improving their safety when used in treating thalassemia and sickle cell patients.

### SUMMARY OF THE INVENTION

The presently disclosed subject matter generally provides enhancer blocking insulators, and certain insulators additionally possess barrier insulator activity. The presently disclosed subject matter also provides expression cassettes comprising one or more insulators and allows for expression of a globin gene (e.g., a human β globin gene). Also provided are vectors comprising such expression cassettes, cells transduced with such expression cassettes or such vectors, and uses of such expression cassettes for treating hemoglobinopathies (e.g., β-thalassemia and sickle cell anemia).

In certain non-limiting embodiments, the presently disclosed subject matter provides an expression cassette comprising a globin gene or a functional portion thereof operably linked to a β-globin locus control region (LCR) that comprises a Dnase I hypersensitive site-2 (HS2) region, a Dnase I hypersensitive site-3 (HS3) region, and a Dnase I hypersensitive site-4 (HS4) region, wherein the HS4 region has a length of less than about 800 bp and does not comprise the nucleotide sequence set forth in SEQ ID NO: 50, which is provided below.

[SEQ ID NO: 50]
TGAGCCCCTTTTCCTCTAACTGAAAGAAGGAAAAAAAAAATGGAACCCAA
AATATTCTACATAGTTTCCATGTCACAGCCAGGGCTGGGCAGTCTCCTGT
TATTTCTTTTAAAA

In certain embodiments, the HS2 region does not comprise the nucleotide sequence set forth in SEQ ID NO: 52, which is provided below.

[SEQ ID NO: 52]
GTATATGTGTATATATATATATATATATTCAGGAAATAATATAT

In certain embodiments, the HS2 region has nucleotides 45-860 of SEQ ID NO: 9. In certain embodiments, the HS2 region has the nucleotide sequence set forth in SEQ ID NO: 33.

In certain embodiments, the expression cassette further comprises a Dnase I hypersensitive site-1 (HS1) region.

The presently disclosed subject matter also provides an expression cassette comprising a globin gene or a functional portion thereof operably linked to a β-globin locus control region (LCR) that comprises a HS1 region, a HS3 region, and a HS4 region and does not comprise a core sequence of HS2 region, wherein the HS4 region has a length of less than about 800 bp and does not comprise the nucleotide sequence set forth in SEQ ID NO: 50. In certain embodiments, the core sequence of HS2 has the nucleotide sequence set forth in SEQ ID NO:20 or SEQ ID NO:21. In certain embodiments, the β-globin LCR does not comprise a HS2 region that sustains the enhancer activity of HS2.

In various of these embodiments, the HS4 region has a length of between about 700 bp and about 800 bp. In certain embodiments, the HS4 region has a length of about 750 bp. In certain embodiments, the HS4 region does not comprise the nucleotide sequence set forth in SEQ ID NO: 51, which is provided below.

[SEQ ID NO: 51]
TTTAATCTAACAATTATGAACAGCAATGAGATAATATGTACAAAGTACCC
AGACCTATGTGGTAGAGCATCAAGGAAGCGCATTGCGGAGCAGTTTTTTG
TTTGTTTGTTTTTGTATTCTGTTTCGTGAGGCAAGGTTTCACTCTGCTGT
CCAGGCTGGAGTGCAGTGGCAAGATCATGTCTCACTGCAGCCTTGAC

In various of these embodiments, the HS3 region has a length of about 1300 bp. In certain embodiments, the HS3 region has the nucleotide sequence set forth in SEQ ID NO: 5. In certain embodiments, the HS3 region has the nucleotide sequence set forth in SEQ ID NO: 34.

In various of these embodiments, the HS1 region has a length of about 600 bp. In certain embodiments, the HS1 region has the nucleotide sequence set forth in SEQ ID NO: 3.

In certain embodiments, the β-globin LCR comprises or consists essentially of a HS2 region having nucleotides 45-860 of SEQ ID NO: 9, a HS3 region having the nucleotide sequence set forth in SEQ ID NO: 5, and a HS4 region having nucleotides 115-868 of SEQ ID NO: 6.

In certain embodiments, the β-globin LCR comprises or consists essentially of a HS2 region having the nucleotide sequence set forth in SEQ ID NO: 33, a HS3 region having the nucleotide sequence set forth in SEQ ID NO: 34, and a HS4 region having the nucleotide sequence set forth in SEQ ID NO: 35.

In certain embodiments, the β-globin LCR comprises or consists essentially of a HS1 region having the nucleotide sequence set forth in SEQ ID NO: 3, a HS3 region having the nucleotide sequence set forth in SEQ ID NO: 5, and a HS4 region having nucleotides 115-868 of SEQ ID NO: 6.

In certain embodiments, the β-globin LCR comprises or consists essentially of a HS1 region having the nucleotide sequence set forth in SEQ ID NO: 3, a HS3 region having the nucleotide sequence set forth in SEQ ID NO: 34, and a HS4 region having the nucleotide sequence set forth in SEQ ID NO: 35.

In certain embodiments, the β-globin LCR comprises or consists essentially of a HS1 region having the nucleotide sequence set forth in SEQ ID NO: 3, a HS2 region having nucleotides 45-860 of SEQ ID NO: 9, a HS3 region having the nucleotide sequence set forth in SEQ ID NO: 5, and a HS4 region having nucleotides 115-868 of SEQ ID NO: 6.

In certain embodiments, the β-globin LCR comprises or consists essentially of a HS1 region having the nucleotide sequence set forth in SEQ ID NO: 3, a HS2 region having the nucleotide sequence set forth in SEQ ID NO: 33, a HS3 region having the nucleotide sequence set forth in SEQ ID NO: 34, and a HS4 region having the nucleotide sequence set forth in SEQ ID NO: 35.

In various of these embodiments, the expression cassette further comprises at least one an insulator comprising the CTCF binding site sequence set forth in SEQ ID NO:18. In certain embodiments, the at least one insulator has the nucleotide sequence set forth in SEQ ID NO: 1, SEQ ID NO: 24, SEQ ID NO: 25, SEQ ID NO: 27, SEQ ID NO: 28, SEQ ID NO: 29, SEQ ID NO: 30, or SEQ ID NO: 31. In certain embodiments, the expression cassette comprises two insulators, each comprising the CTCF binding site sequence set forth in SEQ ID NO:18, for example, but not limited to, where one or both insulators comprise the nucleotide sequence set forth in SEQ ID NO: 1, SEQ ID NO: 24, SEQ ID NO:25, SEQ ID NO: 27, SEQ ID NO: 28, SEQ ID NO: 29, SEQ ID NO: 30, or SEQ ID NO: 31.

In certain embodiments, the expression cassette further comprises at least one erythroid-specific enhancer. In certain embodiments, the at least one erythroid-specific enhancer is positioned within the β-globin LCR. In certain embodiments, the at least one erythroid-specific enhancer is positioned between the HS1 region and the HS3 region of the β-globin LCR. In certain embodiments, the at least one erythroid-specific enhancer has the nucleotide sequence set forth in SEQ ID NO: 13, SEQ ID NO: 14, SEQ ID NO: 15, SEQ ID NO: 16, or SEQ ID NO: 17. In certain embodiments, the at least one erythroid-specific enhancer has the nucleotide sequence set forth in SEQ ID NO: 15. In certain embodiments, the at least one erythroid-specific enhancer is between about 100 and about 200 bp in length. In certain embodiments, the expression cassette comprises one, two or three erythroid-specific enhancers.

The presently disclosed subject matter also provides an expression cassette comprising at least one erythroid-specific enhancer disclosed herein and a globin gene or a functional portion thereof, wherein the expression cassette does not comprise a β-globin locus control region (LCR).

The presently disclosed subject matter further provides an expression cassette comprising at least one erythroid-specific enhancer disclosed herein and a globin gene or a functional portion thereof that is operatively linked to a β-globin locus control region (LCR) that comprises a HS3 region, and does not comprise a core sequence of a HS1 region, a core sequence of a HS2 region, or a core sequence of a HS4 region.

In certain embodiments, the globin gene is selected from the group consisting of β-globin gene, γ-globin gene, and δ-globin gene. In certain non-limiting embodiments, the globin gene is human β-globin gene. In certain non-limiting embodiments, the human β-globin gene is a wild-type human β-globin gene. In certain non-limiting embodiments, the human β-globin gene is a non-wild-type human β-globin gene, including, but not limited to, a human β-globin gene comprising one or more deletions of intron sequences, a human β-globin gene encoding at least one (e.g., one or two) anti-sickling amino acid residue (e.g., a human β-globin gene encoding anti-sickling hemoglobin 1 (HB AS1), and a human β-globin gene encoding anti-sickling hemoglobin 2 (HB AS2)), and a human β-globin gene comprising one or more deletions of intron sequences and encoding at least one anti-sickling amino acid residue. In certain non-limiting embodiments, the human β-globin gene is a human β^{A}-globin gene encoding a threonine to glutamine mutation at codon 87 (β^{A-T87Q}). In certain non-limiting embodiments, the human β-globin gene is a human β^{A}-globin gene encoding a threonine to glutamine mutation at codon 87 (β^{A-T87Q}) and comprising a deletion in intron 2. In certain non-limiting embodiments, the human β-globin gene is a human β^{A}-globin gene encoding a glutamic acid to alanine mutation at codon 22 (β^{A-E22A}). In certain non-limiting embodiments, the human β-globin gene is a human β^{A}-globin gene encoding a glutamic acid to alanine mutation at codon 22 (β^{A-E22A}) and comprising a deletion in intron 2. In certain non-limiting embodiments, the human β-globin gene is a human β^{A}-globin gene encoding an asparagine to lysine mutation at codon 80 (β^{-N80K}). In certain non-limiting embodiments, the human β-globin gene is a human β^{A}-globin gene encoding an asparagine to lysine mutation at codon 80 (β^{-N80K}) and comprising a deletion in intron 2. In certain non-limiting embodiments, the human β-globin gene is a human β^{A}-globin gene encoding a glutamic acid to alanine mutation at codon 22 and an asparagine to lysine mutation at codon 80. In certain non-limiting embodiments, the human β-globin gene is a human β^{A}-globin gene encoding a glutamic acid to alanine mutation at codon 22 and an asparagine to lysine mutation at codon 80 and comprising a deletion in intron 2. In certain non-limiting embodiments, the human β-globin gene is a human β^{A}-globin gene encoding a glutamic acid to alanine mutation at codon 22 and a threonine to glutamine mutation at codon 87. In certain non-limiting embodiments, the human β-globin gene is a human β^{A}-globin gene encoding a glutamic acid to alanine mutation at codon 22 and a threonine to glutamine mutation at codon 87 and comprising a deletion in intron 2. In certain embodiments, the deletion in intro 2 is a deletion of about 370 bp.

In certain embodiments, the expression cassette further comprises a β-globin promoter. In certain embodiments, the β-globin promoter is positioned between the globin gene or functional portion thereof and β-globin LCR. In certain non-limiting embodiments, the β-globin promoter is a human β-globin promoter that is about 265 bp in length. In certain non-limiting embodiments, the β human β-globin promoter has the nucleotide sequence set forth in SEQ ID NO:11.

In certain embodiments, the expression cassette further comprises a human β-globin 3′ enhancer. In certain embodiments, the human β-globin 3′ enhancer is positioned in the upstream of the globin gene or functional portion thereof. In certain embodiments, the β-globin 3′ enhancer is between about 700 and about 900 bp in length, e.g., between about 800 and 900 bp in length. In certain embodiments, the human β-globin 3′ enhancer is about 880 bp in length. In certain embodiments, the human β-globin 3′ enhancer has the nucleotide sequence set forth in SEQ ID NO:12.

In certain embodiments, the expression cassette allows for expression of the globin gene or functional portion thereof in a mammal. In certain embodiments, the expression cassette allows for expression of a human β-globin gene. In certain embodiments, the expression of the globin gene or functional portion thereof is restricted to erythroid tissue.

The presently disclosed subject matter also provides recombinant vectors comprising the above-described expression cassettes. In certain embodiments, the recombinant vector is a retroviral vector. In certain embodiments, the retroviral vector is a lentivirus vector. In certain embodiments, the expression cassette comprised in the recombinant vector comprises one insulator. In certain embodiments, the expression cassette comprised in the recombinant vector comprises two insulators, e.g., two of the insulator comprising the nucleotide sequence set forth in SEQ ID NO: 1, SEQ ID NO: 24, SEQ ID NO: 25, SEQ ID NO: 27, SEQ ID NO: 28, SEQ ID NO: 29, SEQ ID NO: 30, SEQ ID NO: 31, or SEQ ID NO: 32. In certain embodiments, the recombinant vector further comprises a Woodchuck hepatitis post-regulatory element (WPRE) in the 3′ long terminal repeat (LTR) of the vector. In certain embodiments, the recombinant vector further comprises a bovine growth hormone polyadenylation signal in the 3′ long terminal repeat (LTR) of the vector.

In addition, the presently disclosed subject matter provides non-naturally occurring or engineered nucleases comprising the above-described expression cassettes. In certain embodiments, the nuclease is selected from the group consisting of a non-naturally occurring or engineered zinc-finger nuclease (ZFN), a non-naturally occurring or engineered meganuclease, and a non-naturally occurring or engineered transcription activator-like effector nuclease (TALEN). In certain embodiments, the nuclease comprises a DNA binding domain and a nuclease cleavage domain. In certain embodiments, the nuclease binds to a genomic safe harbor site. In certain embodiments, the nuclease generates a double strand break (DSB) at the genomic safe harbor site. In certain embodiments, the expression cassette comprised in the nuclease comprises two insulators, e.g., two of the insulator comprising the nucleotide sequence set forth in SEQ ID NO: 1, SEQ ID NO: 24, SEQ ID NO: 25, SEQ ID NO: 27, SEQ ID NO: 28, SEQ ID NO: 29, SEQ ID NO: 30, SEQ ID NO: 31, or SEQ ID NO: 32. In certain embodiments, the nuclease allows for targeted delivery of the expression cassette. The presently disclosed subject matter also provides polynucleotides encoding the above-described nucleases, and vectors comprising the polynucleotides. In certain embodiments, the vector is a lentiviral vector.

Furthermore, the presently disclosed subject matter provides non-naturally occurring or engineered CRISPR-Cas systems comprising the above-described expression cassettes. In certain embodiments, the CRISPR-Cas system comprises a CRISPR-Cas nuclease and single-guide RNA. In certain embodiments, the CRISPR-Cas system binds to a genomic safe harbor site. In certain embodiments, the CRISPR-Cas system generates a double strand break (DSB) at the genomic safe harbor site. In certain embodiments, the expression cassette comprised in the CRISPR-Cas system comprises two insulators, e.g., two of the insulator comprising the nucleotide sequence set forth in SEQ ID NO: 1, SEQ ID NO: 24, SEQ ID NO: 25, SEQ ID NO: 27, SEQ ID NO: 28, SEQ ID NO: 29, SEQ ID NO: 30, SEQ ID NO: 31, or SEQ ID NO: 32. In certain embodiments, the CRISPR-Cas allows for targeted delivery of the expression cassette. The presently disclosed subject matter also provides polynucleotides encoding the above-described CRISPR-Cas systems, and vectors comprising the polynucleotides. In certain non-limiting embodiments, the vector is a lentiviral vector.

In certain embodiments, the genomic safe harbor site is an extragenic genomic safe harbor site. In certain embodiments, the genomic safe harbor site is located on chromosome 1. In certain embodiments, the genomic safe harbor meets all of the following five criteria: (1) distance of at least 50 kb from the 5′ end of any gene (e.g., from the 5′ end of the gene), (ii) distance of at least 300 kb from any cancer-related gene, (iii) within an open/accessible chromatin structure (measured by DNA cleavage with natural or engineered nucleases), (iv) location outside a gene transcription unit and (v) location outside ultraconserved regions (UCRs), microRNA or long non-coding RNA of the human genome.

Additionally, the presently disclosed subject matter provides cells transduced with the above-described expression cassettes, cells transduced with the above-described recombinant vectors, cells transduced with the above-described nucleases, cells transduced with the above-described CRISPR-Cas systems. In addition, the presently disclosed subject matter provides cells transduced with the above-described vectors. In certain embodiments, the cell is selected from the group consisting of a hematopoietic stem cell, an embryonic stem cell, an induced pluripotent stem cell, and a hemogenic endothelium cell. In certain non-limiting embodiments, the hematopoietic stem cell is a CD34⁺ hematopoietic stem cell. In certain embodiments, the cell is transduced ex vivo.

Also provided are pharmaceutical compositions comprising an effective amount of the above-described cells and a pharmaceutically acceptable carrier. The presently disclosed subject matter also provides pharmaceutical compositions for treating a hemoglobinopathy comprising an effective amount of the above-described cells and a pharmaceutically acceptable carrier.

Furthermore, the presently disclosed subject matter provides kits for treating a hemoglobinopathy comprising the above-described cells. In certain embodiments, the kits further comprise written instructions for using the cell for treating a subject having a hemoglobinopathy.

In addition, the presently disclosed subject matter provides methods of treating a hemoglobinopathy in a subject, comprising administering an effective amount of the above-described cells to the subject, thereby restoring the subject's ability to produce red blood cells containing normal hemoglobin. In certain embodiments, a therapeutically relevant level of hemoglobin is produced in the subject following administering the cell to the subject. In certain amendments, the method comprises administering an effective amount of the cell transduced with the above-described recombinant vector. In certain embodiments, the vector copy number of the recombinant vector in the cell that provides for the therapeutically relevant level of hemoglobin in the subject is about 0.5-2 vector copy number per cell. In certain embodiments, the method corrects ineffective erythropoiesis in the subject. In certain embodiments, the method does not incur the risk of graft-versus-host disease in the subject. In certain embodiments, the method does not comprise administering an immunosuppressive agent. In certain embodiments, the cell is selected from the group consisting of a hematopoietic stem cell, an embryonic stem cell, an induced pluripotent stem cell, and a hemogenic endothelium cell. In certain non-limiting embodiments, the subject is a human. In certain embodiments, the cell is from the subject. In certain non-limiting embodiments, the cell is from bone marrow of the subject.

In accordance with the presently disclosed subject matter, the hemoglobinopathy can be selected from the group consisting of hemoglobin C disease, hemoglobin sickle cell disease (SCD), sickle cell anemia, hereditary anemia, thalassemia, β-thalassemia, thalassemia major, thalassemia intermedia, α-thalassemia, and hemoglobin H disease. In certain non-limiting embodiments, the hemoglobinopathy is β-thalassemia. In certain non-limiting embodiments, the hemoglobinopathy is sickle cell anemia.

### BRIEF DESCRIPTION OF THE FIGURES

The following Detailed Description, given by way of example, but not intended to limit the invention to specific embodiments described, may be understood in conjunction with the accompanying drawings.

FIG. 1 depicts a recombinant vector comprising an expression cassette in accordance with one non-limiting embodiment of the presently disclosed subject matter. “RRE” represents for “Rev Response Element”. cPPT represents for “Central Polypurine tract”.

FIG. 2 depicts a recombinant vector an expression cassette in accordance with one non-limiting embodiment of the presently disclosed subject matter.

FIG. 3 depicts a recombinant vector an expression cassette in accordance with one non-limiting embodiment of the presently disclosed subject matter.

FIG. 4 depicts a recombinant vector an expression cassette in accordance with one non-limiting embodiment of the presently disclosed subject matter.

FIGS. 5A-5C represent the genotoxicity of insulator A1. FIG. 5A demonstrates the gammaretroviral vector genotoxicity assay used. FIG. 5B notices the increased survival of mice receiving 32D cells transduced with insulated gammaretroviral vector. Also notice the results obtained with cHS4 and with the uninsulated control. FIG. 5C shows that insulator A1 decreased the risk of genotoxicity.

FIG. 6 represents normalized B chain expression in thalassemic Hbb^{th3/+} mice 8 and 44 weeks post-treatment.

FIG. 7 represents the evaluation of enhancer activity in non-erythroid K562 cells.

FIG. 8 represents the erythroid-specific enhancers in accordance with certain embodiments of the presently disclosed subject matter (SEQ ID NOS 13-17, respectively, in order of appearance).

FIG. 9 represents the erythroid-specific enhancers in accordance with certain embodiments of the presently disclosed subject matter (SEQ ID NO: 26).

FIGS. 10A-10B depict various recombinant vectors comprising the presently disclosed expression cassettes. FIG. 10A depicts recombinant vector #18, and #49-#55. FIG. 10B shows recombinant vector #18, #40, #49, #52-#55, and #57-#62.

FIG. 11 represents the titer of the recombinant vectors comprising the presently disclosed expression cassettes.

FIG. 12 represents the titer of the recombinant vectors comprising the presently disclosed expression cassettes.

FIG. 13 depicts recombinant vectors comprising expression cassettes in accordance with certain non-limiting embodiments of the presently disclosed subject matter.

FIG. 14 depicts recombinant vectors comprising expression cassettes in accordance with certain non-limiting embodiments of the presently disclosed subject matter.

FIG. 15 depicts recombinant vectors comprising expression cassettes in accordance with certain non-limiting embodiments of the presently disclosed subject matter.

FIG. 16 depicts the average Hb production for vectors (TNS9.B87.A1 SNS23.2.B87.A1, SNS26.B87.A1, and SNS27.2B.87.A1).

FIG. 17 depicts the titration assay relative to various vectors.

FIG. 18 depicts the average of ΔHb normalized per VCN for various vectors.

FIG. 19 depicts the average of total Hemoglobin (Hb) in thalassemic mouse peripheral blood at time points of 6-week, 5-month, and 9-month.

FIG. 20 depicts the average gain in Hb level (ΔHb) normalized to vector copy (VCN) at time points of 6-week, 5-month, and 9-month. ΔHb=Hb level −7.5 (baseline level in thalassemic mice).

### DETAILED DESCRIPTION OF THE INVENTION

The presently disclosed subject matter generally provides expression cassettes that allow for expression of a globin gene (e.g., human β-globin gene). In certain non-limiting embodiments, the expression cassette comprises at least one insulator comprising the CTCF binding site sequence set forth in SEQ ID NO:18, for example, but not limited to, an insulator comprising the nucleotide sequence set forth in SEQ ID NO: 1, SEQ ID NO: 24, SEQ ID NO: 25, or SEQ ID NO: 27 and a globin gene or a functional portion thereof operably linked to a β-globin locus control region (LCR). The expression of the globin gene induced by the presently disclosed expression cassettes is erythroid-specific, differentiation stage-specific, high-level, and sustained. The presently disclosed subject matter also provides recombinant vectors, non-naturally occurring or engineered nucleases, and non-naturally occurring or engineered CRISPR-Cas systems comprising such expression cassettes, and cells transduced with such expression cassettes, recombinant vectors, nucleases and CRISPR-Cas systems. The presently disclosed expression cassettes and vectors comprising thereof provide for a safe gene transfer therapy as therapeutic transgene expression is achieved (e.g., a therapeutically relevant level of hemoglobin is produced) with a low vector copy number per cell (e.g., 0.5-2, 1-2, or even 0.5-1). In addition, the presently disclosed subject matter provides methods of using such transduced cells for treating a hemoglobinopathy (e.g., β-thalassemia and sickle cell anemia).

### I. Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, the term “expression cassette” refers to a nucleic acid construct, generated recombinantly or synthetically, with a series of specified nucleic acid elements, which permit transcription of a particular nucleic acid in a target cell. The expression cassette can be incorporated into a plasmid, chromosome, mitochondrial DNA, plastid DNA, virus or nucleic acid region. The expression cassette portion can include a gene to be transcribed and elements that control the expression of the gene (e.g., a promoter).

As used herein, the term “β-globin locus control region (LCR)” refers to a polynucleotide composed of one or more Dnase I hypersensitive site (HS) regions, including a HS1 region, a HS2 region, a HS3 region, and a HS4 region. The structure of many LCRs of the β-globin genes have been published, e.g., human (Li et al., J. Biol. Chem. (1985); 260:14,901; Li et al., Proc. Natl. Acad. Sci. (1990) 87:8207); mouse (Shehee et al., J. Mol. Biol. (1989); 205:41); rabbit (Margot et al., J. Mol. Biol. (1989); 205:15); and goat (Li, Q., et al., Genomics (1991); 9:488), each of which are incorporated by reference herein. In certain embodiments, the β-globin LCR comprises a HS2 region (e.g., a β-globin LCR comprising a HS2 region, a HS3 region and a HS4 region; and a β-globin LCR comprising a HS1 region, a HS2 region, a HS3 region and a HS4 region). In certain embodiments, the β-globin LCR does not comprise a HS2 region (e.g., a β-globin LCR comprising or consisting essentially of a HS1 region, a HS3 region, a HS4 region). In certain embodiments, the β-globin LCR does not comprise a HS2 region or a HS1 region (e.g., a β-globin LCR comprising or consisting essentially of a HS3 region and a HS4 region). In certain embodiments, the β-globin LCR does not comprise a HS1 region, a HS2 region or a HS4 region (e.g., a β-globin LCR comprising or consisting essentially of a HS3 region).

As used herein, the term “recombinant” includes reference to a cell or vector, that has been modified by the introduction of a heterologous nucleic acid or that the cell is derived from a cell so modified. Thus, for example, recombinant cells express genes that are not found in identical form within the native (non-recombinant) form of the cell or express native genes that are otherwise abnormally expressed, under expressed or not expressed at all as a result of deliberate human intervention or may have reduced or eliminated expression of a native gene.

As used herein, the term “globin” refers to a family of heme-containing proteins that are involved in the binding and transport of oxygen. Subunits of vertebrate and invertebrate hemoglobins, vertebrate and invertebrate myoglobins or mutants thereof are included by the term globin.

As used herein, the term “wild-type” refers to the normal gene, virus, or organism found in nature without any mutation or modification.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene region, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide can comprise one or more modified nucleotides, such as methylated nucleotides and nucleotide analogs. In particular embodiments, the presently disclosed subject matter provides polynucleotides encoding one or more globin genes or functional portions thereof. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. Such polynucleotides need not be about 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration can ordinarily be less than about 750 mM NaCl and 75 mM trisodium citrate, e.g., less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide, e.g., at least about 50% formamide. Stringent temperature conditions can ordinarily include temperatures of at least about 30° C., e.g., of at least about 37° C. or of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In certain embodiments, hybridization occurs at 37° C. in 500 mM NaCl, 50 mM trisodium citrate, 1% SDS, 35% formamide, and 100 μg/ml denatured salmon sperm DNA (ssDNA). In certain embodiments, hybridization occurs at 42° C. in 250 mM NaCl, 25 mM trisodium citrate, 1% SDS, 50% formamide, and 200 μg/ml ssDNA. Useful variations on these conditions can be readily apparent to those skilled in the art.

For most applications, washing steps that follow hybridization can also vary in stringency. Wash stringency conditions can be defined by salt concentration and by temperature. As above, wash stringency can be increased by decreasing salt concentration or by increasing temperature. For example, stringent salt concentration for the wash steps can be less than about 30 mM NaCl and 3 mM trisodium citrate, e.g., less than about 15 mM NaCl and 1.5 mM trisodium citrate. Stringent temperature conditions for the wash steps can ordinarily include a temperature of at least about 25° C., e.g., of at least about 42° C., or of at least about 68° C. In certain embodiments, wash steps occur at 25° C. in 30 mM NaCl, 3 mM trisodium citrate, and 0.1% SDS. In certain embodiments, wash steps occur at 42° C. in 15 mM NaCl, 1.5 mM trisodium citrate, and 0.1% SDS. In certain embodiments, wash steps occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Rogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

As used herein, the terms “polypeptide” and “protein” are used interchangeably to refer to a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers. Particular embodiments of the presently disclosed subject matter also include polypeptide “variants.” Polypeptide “variant” refers to polypeptides that are distinguished from a reference polypeptide by the addition, deletion, truncations, and/or substitution of at least one amino acid residue, and that retain a biological activity. In certain embodiments, a polypeptide variant is distinguished from a reference polypeptide by one or more substitutions, which may be conservative or non-conservative, as known in the art. In certain embodiments, a variant polypeptide includes an amino acid sequence having at least about 50%, 55%, 60%, 65%, 70%, 75%, 80%, 85%, 90%, 91%, 92%, 93%, 94%, 95%, 96%, 97%, 98%, 99% or more sequence identity or similarity to a corresponding sequence of a reference polypeptide. In certain embodiments, the amino acid additions or deletions occur at the C-terminal end and/or the N-terminal end of the reference polypeptide. In certain embodiments, the amino acid deletions include C-terminal truncations of about 1, about 2, about 3, about 4, about 5, about 6, about 7, about 8, about 9, about 10, about 15, about 20, about 25, about 30, about 35, about 40, about 45, 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, or about 175 or more amino acids, including all intervening numbers of amino acids, e.g., 25, 26, 27, 29, 30 . . . 100, 101, 102, 103, 104, 105 . . . 170, 171, 172, 173, 174, etc.

As noted above, polypeptides of the presently disclosed subject matter may be altered in various ways including amino acid substitutions, deletions, truncations, and insertions. Methods for such manipulations are generally known in the art. For example, amino acid sequence variants of a reference polypeptide can be prepared by mutations in the DNA. Methods for mutagenesis and nucleotide sequence alterations are well known in the art. See, for example, Kunkel (1985, Proc. Natl. Acad. Sci. USA. 82: 488-492), Kunkel et al., (1987, Methods in Enzymol, 154: 367-382), U.S. Pat. No. 4,873,192, Watson, J. D. et al., Molecular Biology of the Gene, Fourth Edition, Benjamin/Cummings, Menlo Park, Calif, 1987) and the references cited therein. Guidance as to appropriate amino acid substitutions that do not affect biological activity of the protein of interest may be found in the model of Dayhoff et al., (1978) Atlas of Protein Sequence and Structure (Natl. Biomed. Res. Found., Washington, D.C.).

As used herein, the term “substantially identical” refers to a polypeptide or a polynucleotide exhibiting at least 50% identity to a reference amino acid sequence (for example, any one of the amino acid sequences described herein) or a nucleic acid sequence (for example, any one of the nucleic acid sequences described herein). Preferably, such a sequence is at least about 60%, e.g., about 80%, about 85%, about 90%, about 95%, or about 99% identical at the amino acid level or nucleic acid to the sequence used for comparison.

Sequence identity or homology is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. In an exemplary approach to determining the degree of identity or homology, a BLAST program may be used, with a probability score between e−3 and e−100 indicating a closely related sequence. The percentage of identity between two sequences can also be determined with programs such as DNAMAN (Lynnon Biosoft, version 3.2). Using this program two sequences can be. aligned using the optimal alignment algorithm (Smith and Waterman, 1981). After alignment of the two sequences the percentage identity can be calculated by dividing the number of identical nucleotides between the two sequences by the length of the aligned sequences minus the length of all gaps.

Terms that describe the orientation of polynucleotides include: 5′ (normally the end of the polynucleotide having a free phosphate group) and 3′ (normally the end of the polynucleotide having a free hydroxyl (OH) group). Polynucleotide sequences can be annotated in the 5′ to 3′ orientation or the 3′ to 5′ orientation.

As used herein, a “single guide RNA” or a “synthetic guide RNA” refers to the polynucleotide sequence comprising the guide sequence, the tracr sequence and the tracr mate sequence. The term “guide sequence” refers to the about 20 bp sequence within the guide RNA that specifies the target site and may be used interchangeably with the terms “guide” or “spacer”. The term “tracr mate sequence” may also be used interchangeably with the term “direct repeat(s)”.

The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

As used herein, the term “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

As used herein, the term “treating” or “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

As used herein, the term “subject” or “individual” refers to a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys. The term “immunocompromised” as used herein refers to a subject who has an immunodeficiency. The subject is very vulnerable to opportunistic infections, infections caused by organisms that usually do not cause disease in a person with a healthy immune system, but can affect people with a poorly functioning or suppressed immune system.

As used herein, the term “isolated cell” refers to a cell that is separated from the molecular and/or cellular components that naturally accompany the cell. As used herein, the term “isolated” refers to material that is free, substantially free, or essentially free to varying degrees from components which normally accompany it as found in its native state. “Isolate” denotes a degree of separation from original source or surroundings.

As used herein, the term “cell population” refers to a group of at least two cells expressing similar or different phenotypes. In non-limiting examples, a cell population can include at least about 10, at least about 100, at least about 200, at least about 300, at least about 400, at least about 500, at least about 600, at least about 700, at least about 800, at least about 900, at least about 10³ cells, at least about 10⁴ cells, at least about 10⁵ cells, at least about 10⁶ cells, at least about 10⁷ cells, or at least about 10⁸ cells expressing similar or different phenotypes.

As used herein, the term “cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, fusion polypeptides are used for targeted double-stranded DNA cleavage.

As used herein, the term “cleavage half-domain” refers to a polypeptide sequence which, in conjunction with a second polypeptide (either identical or different) forms a complex having cleavage activity (preferably double-strand cleavage activity). The terms “first and second cleavage half-domains;” “+ and − cleavage half-domains” and “right and left cleavage half-domains” are used interchangeably to refer to pairs of cleavage half-domains that dimerize.

As used herein, the term “chromosome” refers to a chromatin complex comprising all or a portion of the genome of a cell. The genome of a cell is often characterized by its karyotype, which is the collection of all the chromosomes that comprise the genome of the cell. The genome of a cell can comprise one or more chromosomes.

As used herein, the term “gene” includes a DNA region encoding a gene product, as well as all DNA regions which regulate the production of the gene product, whether or not such regulatory sequences are adjacent to coding and/or transcribed sequences. Accordingly, a gene includes, but is not limited to, promoter sequences, terminators, translational regulatory sequences such as ribosome binding sites and internal ribosome entry sites, enhancers, silencers, insulators, boundary elements, replication origins, matrix attachment sites and locus control regions

The terms “operative linkage” and “operatively linked” (or “operably linked”) are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous.

A “functional region” or “functional portion” of a protein, polypeptide or nucleic acid is a protein, polypeptide or nucleic acid whose sequence is not identical to the full-length protein, polypeptide or nucleic acid, yet retains the same function as the full-length protein, polypeptide or nucleic acid. A functional region can possess more, fewer, or the same number of residues as the corresponding native molecule, and/or can contain one or more amino acid or nucleotide substitutions. Methods for determining the function of a nucleic acid (e.g., coding function, ability to hybridize to another nucleic acid) are well-known in the art. Similarly, methods for determining protein function are well-known. For example, the DNA-binding function of a polypeptide can be determined, for example, by filter-binding, electrophoretic mobility-shift, or immunoprecipitation assays. DNA cleavage can be assayed by gel electrophoresis. The ability of a protein to interact with another protein can be determined, for example, by co-immunoprecipitation, two-hybrid assays or complementation, both genetic and biochemical.

As used herein, the term “promoter” refers to a recognition site of a polynucleotide (DNA or RNA) to which an RNA polymerase binds. The term “enhancer” refers to a segment of DNA which contains sequences capable of providing enhanced transcription and in some instances can function independent of their orientation relative to another control sequence. An enhancer can function cooperatively or additively with promoters and/or other enhancer elements.

As used herein, the term “vector” refers to any genetic element, such as a plasmid, phage, transposon, cosmid, chromosome, virus, virion, etc., which is capable of replication when associated with the proper control elements and which can transfer gene sequences into cells. Thus, the term includes cloning and expression vehicles, as well as viral vectors and plasmid vectors.

As used herein, the term “modulate” refers to altering positively or negatively. Exemplary modulations include an about 1%, about 2%, about 5%, about 10%, about 25%, about 50%, about 75%, or about 100% change.

As used herein, the term “increase” refers to alter positively by at least about 5%, including, but not limited to, alter positively by about 5%, by about 10%, by about 25%, by about 30%, by about 50%, by about 75%, or by about 100%.

As used herein, the term “reduce” refers to alter negatively by at least about 5% including, but not limited to, alter negatively by about 5%, by about 10%, by about 25%, by about 30%, by about 50%, by about 75%, or by about 100%.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value.

### II. Insulators

Several cases of vector-related malignant transformation have been reported in clinical settings, associated with the activation of cellular oncogenes by vector-encoded enhancers (Baum et al. (2006), Nienhuis et al. (2006), Ramezani et al. (2006)) and various vector modifications have been performed or proposed to reduce vector genotoxicity (Baum et al. (2006), Nienhuis et al. (2006), Ramezani et al. (2006)). A class of DNA elements known as chromatin insulators has been recognized as one approach to improve vector safety and performance (Emery (2011)).

Insulators are naturally occurring DNA elements that help from the functional boundaries between adjacent chromatin domains. Insulators bind proteins that modify chromatin and alter regional gene expression. The placement of insulators in the vectors described herein offer various potential benefits including, but not limited to, 1) shielding of the vector from positional effect variegation of expression by flanking chromosomes (i.e., barrier activity, which may decrease position effects and vector silencing); and 2) shielding flanking chromosomes from insertional trans-activation of endogenous gene expression by the vector (enhancer blocking). There are two basic classes of chromatin insulators: (a) barrier insulators that block the encroachment of silencing heterochromatin into adjoining regions of open chromatin that are transcriptionally permissive, and (b) enhancer blocking insulators that prevent enhancer-mediated transcriptional activation of adjoining regions. The sequences that mediate these activities are physically separable and mechanistically distinct (Recillas-Targa et al. (2002)). Chromatin insulators do not exhibit inherent transcriptional enhancing or repressing activities on their own. As such, they make ideal elements for reducing the interaction between gene transfer vectors and the target cell genome. Insulators can help to preserve the independent function of genes or transcription units embedded in a genome or genetic context in which their expression may otherwise be influenced by regulatory signals within the genome or genetic context (see, e.g., Burgess-Beusse et al. (2002) Proc. Nat'l Acad. Sci. USA, 99:16433; and Zhan et al. (2001) Hum. Genet., 109: 471).

The problems created by insertional mutagenesis of viral vectors are widely known (Nienhuis (2013), Baum et al. (2006), Nienhuis et al. (2006)) as is the evidence that the risks of genotoxicity can be reduced by the use of chromatin insulators (Arumugam et al. (2007), Emery (2011), Evans-Galea et al. (2007), Rivella et al. (2000), Emery et al. (2000), Emery et al. (2002), Yannaki et al. (2002), Hino et al. (2004), Ramezani et al. (2003), Ramezani et al. (2008)). The presently disclosed subject matter provides novel insulators that are powerful enhancer blocking insulators, and certain insulators additionally possess barrier insulator activity. In vertebrates, the function of enhancer blocking insulators is mediated through the zinc-finger DNA-binding factor CTCF (Gaszner and Felsenfeld (2006), Wallace and Felsenfeld (2007)). In general, these elements are thought to function through physical loop structures, which are established by CTCF-mediated interactions between adjacent insulator elements or through CTCF-mediated tethering of the chromatin fiber to structural elements within the nucleus. The first characterized vertebrate chromatin insulator is located within the chicken β-globin locus control region. This element, which contains a DNase-I hypersensitive site-4 (cHS4), appears to constitute the 5′ boundary of the chicken β-globin locus (Prioleau et al. (1999) EMBO J. 18: 4035-4048). A 1.2-kb region containing the cHS4 element displays classic insulator activities, including the ability to block the interaction of globin gene promoters and enhancers in cell lines (Chung et al. (1993) Cell, 74: 505-514), and the ability to protect expression cassettes in Drosophila (Id.), transformed cell lines (Pikaart et al. (1998) Genes Dev. 12: 2852-2862), and transgenic mammals (Wang et al. (1997) Nat. Biotechnol., 15: 239-243; Taboit-Dameron et al. (1999) Transgenic Res., 8: 223-235) from position effects. Much of this activity is contained in a 250-bp region. Within this stretch is a 49-bp cHS4 element (Chung et al. (1997) Proc. Natl. Acad. Sci., USA, 94: 575-580) that interacts with the zinc finger DNA binding protein CTCF implicated in enhancer-blocking assays (Bell et al. (1999) Cell, 98: 387-396).

Insulators, such as cHS4, can block the interaction between enhancers and promoters when placed between these elements (Evans-Galea et al. (2007), Chung et al. (1997), Bell et al. (1999), Ryu et al. (2007), Ryu et al. (2008)). Several studies have demonstrated the ability of the cHS4 insulator to reduce position-effect silencing of gammaretroviral vectors (Evans-Galea et al. (2007), Rivella et al. (2000), Emery et al. (2000), Emery et al. (2002), Yannaki et al. (2002), Hino et al. (2004), Ramezani et al. (2006), Yao et al. (2003), Nishino et al. (2006), Aker et al. (2007), Li and Emery (2008)), and lentiviral vectors (Bank et al. (2005), Arumugam et al. (2007), Puthenveetil et al. (2004), Evans-Galea et al. (2007), Ramezani et al. (2003), Aker et al. (2007), Ma et al. (2003), Chang et al. (2005), Pluta et al. (2005)). Those appropriately designed studies demonstrated that inclusion of the 1.2 kb version of the cHS4 insulator increased the likelihood and/or consistency of vector transgene expression in at least some settings (Arumugam et al. (2007), Emery (2011), Evans-Galea et al. (2007), Emery et al. (2002), Yannaki et al. (2002), Hino et al. (2004), Ramezani et al. (2006), Aker et al. (2007), Li and Emery (2008), Pluta et al. (2005). Jakobsson et al. (2004)). Nevertheless, the degree of protection afforded by the cHS4 insulator is far from complete. In addition, the inclusion of the 1.2 Kb cHS4 can adversely affect vector titers while the smallest cHS4 core has been proven ineffective (Aker et al. (2007), Jakobsson et al. (2004)). By contrast, the insulators of the presently disclosed subject matter do not affect adversely the titers of viral vectors, and are more powerful and effective than the cHS4 insulator.

The presently disclosed insulators are identified through genomic approaches, e.g., using genomic approaches to identify insulators that are powerful enhancer blockers as well as barrier insulators of the human genome. The presently disclosed insulators enhance the safety of gene therapy (e.g., stem cell gene therapy, globin gene therapy). For gene therapy of the hemoglobinopathies, powerful enhancers are required to achieve therapeutic levels of globin gene expression. Powerful insulators therefore represent one means to protect the genomic environment from the powerful enhancers of the integrating vectors.

The presently disclosed insulators possess powerful enhancer blocking activity. For example, and not by way of limitation, an insulator of the present disclosure can reduce the activity of an enhancer element by at least about 10%, at least about 20%, at least about 30%, at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%. In certain embodiments, the insulators possess barrier activity in addition to enhancer blocking activity. The presently disclosed insulators substantially decrease the risks of insertional mutagenesis and genotoxicity associated with viral vectors. Furthermore, when at least one presently disclosed insulator is incorporated into a vector, the insulator(s) does not adversely effect vector titers of the vector. In certain embodiments, the presently disclosed insulators (e.g., insulator A1) increase the in vivo expression of the globin gene or functional portion thereof.

In certain embodiments, the insulator comprises a Transcriptional repressor CTCF binding site, which has the nucleotide sequence set forth in SEQ ID NO: 18, which is provided below:

[SEQ ID NO: 18]
CACCAGGTGGCGCT.

In certain embodiments, the insulator has the nucleotide sequence set forth in SEQ ID NO:1, which is provided below, or a sequence which is at least about 80%, about 85%, about 90%, about 95%, about 98, about 99% or about 100% % identical (homologous), to SEQ ID NO:1. SEQ ID NO: 1 is provided below.

[SEQ ID NO: 1]
TCCTTCCTTTCTAAATGACGAGAGAGACAGAAGAATTCTTCAAGGTTAGT
GTGTCCAGCATGCAACCTTTCCTTCCTGGATGAGCATCCCTGGAGTAGGA
GAGCCAGCCTGCCTCCTGCGCTGGCACAGAGCCCGGTTCCCTAGACAACT
GCCTCTCCAAATCTGATGTCCAGCGCCACCTGGTGTCCACATCAAGCAGA
CACAATTAATAGTCAACCTGTTCAGGAAAACTGTGAGGGGGAAAAAAAAG
AAAGAGGATTTATGAAGGGAAAAGAAAGTTTAGAGGATATGCCACGATTG
GCTAG

In certain embodiments, the insulator comprises or has the nucleotide sequence as set forth in SEQ ID NO:24, or a sequence which is at least about 80%, about 85%, about 90%, about 95%, about 98, about 99% or about 100% identical (homologous), to SEQ ID NO: 24. SEQ ID NO: 24 is provided below.

[SEQ ID NO: 24]
CCAATC GTGGCATATC CTCTAAACTT TCTTTTCCCT
TCATAAATCC TCTTTCTTTT TTTTCCCCCT CACAGTTTTC
CTGAACAGGT TGACTATTAA TTGTGTCTGC TTGATGTGGA
CACCAGGTGG CGCTGGACAT CAGATTTGGA GAGGCAGTTG
TCTAGGGAAC CGGGCTCTGT GCCAGCGCAG GAGGCAGGCT
GGCTCTCCTA TTCCAGGGAT GCTCATCCAG GAAGGAAAGG
TTGCATGCTG GACACACTAA CCTTGAAGAA TTCTTCTGTC
TCTCTCGTCA TTTAGAAAGG AAGGA.

In certain embodiments, the insulator comprises or has the nucleotide sequence as set forth in SEQ ID NO:25 (which is the reverse complement of SEQ ID NO: 1), or a sequence which is at least about 80%, about 85%, about 90%, about 95%, about 98, about 99% or about 100% identical (homologous), to SEQ ID NO: 25. SEQ ID NO: 25 is provided below.

[SEQ ID NO: 25]
CTAGCCAATCGTGGCATATCCTCTAAACTTTCTTTTCCCTTCATAAATCC
TCTTTCTTTTTTTTCCCCCTCACAGTTTTCCTGAACAGGTTGACTATTAA
TTGTGTCTGCTTGATGTGGACACCAGGTGGCGCTGGACATCAGATTTGGA
GAGGCAGTTGTCTAGGGAACCGGGCTCTGTGCCAGCGCAGGAGGCAGGCT
GGCTCTCCTACTCCAGGGATGCTCATCCAGGAAGGAAAGGTTGCATGCTG
GACACACTAACCTTGAAGAATTCTTCTGTCTCTCTCGTCATTTAGAAAGG
AAGGA

In certain embodiments, the insulator comprises or has the nucleotide sequence as set forth in SEQ ID NO: 27, or a sequence which is at least about 80%, about 85%, about 90%, about 95%, about 98, about 99% or about 100% identical (homologous), to SEQ ID NO: 27. SEQ ID NO: 27 is provided below.

[SEQ ID NO: 27]
ctggttctac tcattacatt ccaatcgtgg catatcctct
aaactttctt ttcccttcat aaatcctctt tctttttttt
ccccctcaca gttttcctga acaggttgac tattaattgt
gtctgcttga tgtggacacc aggtggcgct ggacatcaga
tttggagagg cagttgtcta gggaaccggg ctctgtgcca
gcgcaggagg caggctggct ctcctattcc agggatgctc
atccaggaag gaaaggttgc atgctggaca cactaacctt
gaagaattct tctgtctctc tcgtcattta gaaaggaagg

In certain embodiments, the insulator comprises or has the nucleotide sequence as set forth in SEQ ID NO: 28, or a sequence which is at least about 80%, about 85%, about 90%, about 95%, about 98, about 99% or about 100% identical (homologous), to SEQ ID NO: 28. SEQ ID NO: 28 is provided below.

[SEQ ID NO: 28]
CCAATC GTGGCATATC CTCTAAACTT TCTTTTCCCT
TCATAAATCC TCTTTCTTTT TTTTCCCCCT CACAGTTTTC
CTGAACAGGT TGACTATTAA TTGTGTCTGC TTGATGTGGA
CACCAGGTGG CGCTGGACAT CAGATTTGGA GAGGCAGTTG
TCTAGGGAAC CGGGCTCTGT GCCAGCGCAG GAGGCAGGCT
GGCTCTCCTA TTCCAGGGAT GCTCATCCAG GAAGGAAAGG
TTGCATGCTG GACACACTAA CCTTGAAGAA TTCTTCTGTC
TCTCTCGTCA TTTAGAAAGG AAGG

In certain embodiments, the insulator comprises or has the nucleotide sequence as set forth in hg18 coordinates 76229933 to 76230115 of chromosome 1.

In certain embodiments, the insulator comprises or has the nucleotide sequence between residues 68041 and 68160 (or SEQ ID NO: 29 provided below), or between residues and 68041 and 68210 (or SEQ ID NO: 30 provided below), or between residues 68041 and 68280 (or SEQ ID NO: 31 provided below), or between residues 68005 and 68305 (or SEQ ID NO: 24), of Homo sapiens chromosome 1 clone RP11-550H2, GenBank Accession No. AC092813.2, or a sequence at least about 80%, about 85%, about 90%, about 95%, about 98, about 99% or about 100% identical (homologous) thereto.

(SEQ ID NO: 29)
TCATAAATCC TCTTTCTTTT TTTTCCCCCT CACAGTTTTC
CTGAACAGGT TGACTATTAA TTGTGTCTGC TTGATGTGGA
CACCAGGTGG CGCTGGACAT CAGATTTGGA GAGGCAGTTG
(SEQ ID NO: 30)
TCATAAATCC TCTTTCTTTT TTTTCCCCCT CACAGTTTTC
CTGAACAGGT TGACTATTAA TTGTGTCTGC TTGATGTGGA
CACCAGGTGG CGCTGGACAT CAGATTTGGA GAGGCAGTTG
TCTAGGGAAC CGGGCTCTGT GCCAGCGCAG GAGGCAGGCT
GGCTCTCCTA
(SEQ ID NO: 31)
TCATAAATCC TCTTTCTTTT TTTTCCCCCT CACAGTTTTC
CTGAACAGGT TGACTATTAA TTGTGTCTGC TTGATGTGGA
CACCAGGTGG CGCTGGACAT CAGATTTGGA GAGGCAGTTG
TCTAGGGAAC CGGGCTCTGT GCCAGCGCAG GAGGCAGGCT
GGCTCTCCTA TTCCAGGGAT GCTCATCCAG GAAGGAAAGG
TTGCATGCTG GACACACTAA CCTTGAAGAA TTCTTCTGTC

### III. Expression Cassettes

The presently disclosed subject matter provides expression cassettes comprising one or more insulators. In certain embodiments, an expression cassette comprises at least one insulator disclosed in Section II, and a globin gene or a functional portion thereof. In certain embodiments, an expression cassette comprises at least one insulator comprising or having the nucleotide sequence set forth in SEQ ID NO: 1, SEQ ID NO: 24, SEQ ID NO: 25, SEQ ID NO: 27, SEQ ID NO: 28, SEQ ID NO: 29, SEQ ID NO: 30, or SEQ ID NO: 31, and a globin gene or a functional portion thereof.

In certain embodiments, an expression cassette comprises a globin gene or a functional portion thereof, and at least one insulator disclosed in International Patent Publication No. WO2015/138852. WO2015/138852 is herein incorporated by reference in its entirety. In certain embodiments, an expression cassette comprises a globin gene or a functional portion thereof operably and at least one insulator comprising or having the nucleotide sequence set forth in SEQ ID NO: 32, or a fragment thereof (e.g., nucleotides 1 to 320, 10 to 320, 20 to 320, 21 to 320, 55 to 320, 55 to 300, 57 to 300, 57 to 296, 57 to 226, 57 to 200, or 57 to 176 of SEQ ID NO: 32). SEQ ID NO:32 is provided below.

(SEQ ID NO: 32)
CTGGTTCTAC TCATTACATT CCAATCGTGG CATATCCTCT AAACTTTCTT TTCCCTTCAT 60
AAATCCTCTT TCTTTTTTTT CCCCCTCACA GTTTTCCTGA ACAGGTTGAC TATTAATTGT 120
GTCTGCTTGA TGTGGACACC AGGTGGCGCT GGACATCAGA TTTGGAGAGG CAGTTGTCTA 180
GGGAACCGGG CTCTGTGCCA GCGCAGGAGG CAGGCTGGCT CTCCTATTCC AGGGATGCTC 240
ATCCAGGAAG GAAAGGTTGC ATGCTGGACA CACTAACCTT GAAGAATTCT TCTGTCTCTC 300
TCGTCATTTA GAAAGGAAGG 320

In certain embodiments, the globin gene or functional portion thereof is operably linked to a β-globin LCR, e.g., a β-globin LCR disclosed in section 3.1.

In certain embodiments, the expression cassette does not comprise a β-globin LCR. In certain embodiments, the expression cassette comprises at least one insulator (e.g., one disclosed in Section II and 3.6), and does not comprise a β-globin LCR. In certain embodiments, the expression cassette comprises at least one insulator comprising or having the nucleotide sequence set forth in SEQ ID NO: 1, and does not comprise a β-globin LCR. In certain embodiments, the expression cassette comprises an erythroid-specific enhancer (e.g., an erythroid-specific enhancer disclosed in section 3.5) and does not comprise a β-globin LCR. In certain embodiments, the expression cassette comprises an erythroid-specific enhancer having the nucleotide sequence set forth in SEQ ID NO:15, and does not comprise a β-globin LCR. In certain embodiments, the expression cassette comprises at least one insulator comprising or having the nucleotide sequence set forth in SEQ ID NO: 1, and an erythroid-specific enhancer having the nucleotide sequence set forth in SEQ ID NO: 15, and does not comprise a β-globin LCR.

### 3.1. β-Globin LCR

The human β-globin gene cluster consists of five genes embedded within one of many olfactory receptor gene arrays (Bulger et al., PNAS (1999); 96:5129-5134). The cluster spans over 80 kb on chromosome 11p15.4, and includes the five expressed β-like genes and cis-acting regulatory elements that direct their stage-specific expression during ontogeny (Forget (2001), Molecular Mechanism of Beta Thalassemia. Steinberg M H et al., Eds. Disorders of Hemoglobin. Genetics, Pathophysiology and Clinical Management, Cambridge University Press, Cambridge). The genes are arranged in the order of their developmental expression (Stamatoyannopoulos et al., (2001) Hemoglobin Switching. In: Stamatoyannopoulos G, et al., Eds. Molecular Basis of Blood Disorders, W.B. Saunders, Philadelphia, PA), 5′-ε-^{G}γ-^{A}γ-ψη-δ-β-3′. The α-like globin gene cluster (5′-ξ2-ψξ 1-ψα2-ψα1-α2-α1-θ-3′) is located very close to the telomere of the short arm of chromosome 16 and spans about 40 kb. The expression of genes encoded within these two independent clusters is limited to erythroid cells and balanced so that the output of the β-globin-like chains matches that of the α-chains. This fine-tuned balance is regulated at the transcriptional, posttranscriptional and posttranslational levels.

Developmental stage-specific expression is controlled by a number of proximal or distal cis-acting elements and the transcriptional factors that bind to them. In the case of the β-globin gene (HBB), the proximal regulatory elements comprise the β-globin promoter and two downstream enhancers, one located in the second intron of β-globin and the other approximately 800 bp downstream of the gene (Antoniou et al., EMBO J. (1988); 7:377-384; Trudel et al., Genes Dev. (1987); 1:954-961; Trudel et al., Mol. Cell. Biol. (1987); 7:4024-4029). The most prominent distal regulatory element is the β-globin LCR, located 50-60 kb upstream of the HBB and composed of several sub-regions with heightened sensitivity to DNasel in erythroid cells (Forget (2001); Grosveld et al., Cell (1987); 51:975-985; Talbot et al., Nature (1989); 338:352). The most prominent property of the LCR is its strong, transcription-enhancing activity. An exemplary nucleotide sequence of the human β-globin region on chromosome 11 is set forth in SEQ ID NO:19 (GenBank Access No.: NG_000007.3), which is provided below:

[SEQ ID NO: 19]
ggatcctcacatgagttcagtatataattgtaacagaataaaaaatcaattatgtattcaagttgctagtgtcttaagaggttcacatt
tttatctaactgattatcacaaaaatacttcgagttacttttcattataattcctgactacacatgaagagactgacacgtaggtgcct
tacttaggtaggttaagtaatttatccaaaaccacacaatgtagaacctaagctgattcggccatagaaacacaatatgtggtataaat
gagacagagggatttctctccttcctatgctgtcagatgaatactgagatagaatatttagttcatctatcacacattaaacgggactt
tacatttctgtctgttgaagatttgggtgtggggataactcaaggtatcatatccaagggatggatgaaggcaggtgactctaacagaa
agggaaaggatgttggcaaggctatgttcatgaaagtatatgtaaaatccacattaagcttctttctgcatgcattggcaatgtttatg
aataatgtgtatgtaaaagtgtgctgtatattcaaaagtgtttcatgtgcctaggggtgtcaaatactttgagtttgtaagtatatact
tctctgtaatgtgtctgaatatctctatttacttgattctcaataagtaggtatcatagtgaacatctgacaaatgtttgaggaacaat
ttagtgtttacctattcaccaaaatttattaaatgcctaatctgtatcagatatacaattatctggcgaaatctgtaattcctaattta
aacagctgtgtagcctaattagggataaaggcatgcaaacccataatttgtgtaggttgaaatgagctatagaaaaatgcagtatattt
atcagaagtctttagggtcatgaaaaggaatggtcaactgacactgccagggactcatatgtaagagataactaatgtgaagtgacttt
aaaggagaaattagcagaagttttctttccatgtctcctcatcatgttacaataacggaagagattaaaacaacaaatacatttagaca
gcaatgtttatcctggttagatgttttaatctaaatctatcttggagtgttaaaatgcatttgctcacctactttaaaatataaatgaa
ggtaggaacctgtagatacaaaaagttggagaaaaaaagacaataaagatgacaaaaatctattaatccttgatagaaaatgagaagag
ataaaacactggtttacataaagaaaataagatggatagatagcagatccttataaaagtgataatttgagaaaaaaaatactccatat
tctgagtttcttcacataaaataatacaaatctgctgtggtaagttacaaagagatagattttttatcattatataaaagatattttaa
acagagttatacaacaaaggaacagactatgtcatatattctcacttatcactataaacatctcagaaaaatctgcaaaatcatttcat
agcattttaaatagttaggaataatgtagaaaactgaaacagttctaagtttcccacaaacttagagtctcaaatgttgcattacctaa
cttacctgcaaatattttatacaaatttgcacatgctactctagtcaaaaatatatgtacattatgggtattttctgtgtgtaacttgg
ttctagttgcttctttcagaaatagcctctatttttgatttacctgataaaatcacattcctctccaaagccttctaaatacttccaga
ctaactactttttagtacatctaagaagaaaagagttttgtctcttatccacctctgagtcaaaaagcagcatgtccatcaattggtac
atagttcccacagccccacttagctctggattggagttctacttggcattgtttgcaactacatggacgtaaaatgcatggattctctt
gaaaaaatgtttctgccatgatgttctctgaaagagactaaccttccctcgctttgcagagaaagactcgtgtaatccttgacaatgtc
atctcatctatttattcccatgtctacccatatgtgaccttcatgtctttgctctaagcccctacatcctcaatctacacactaggata
gtataaaagtaatagtaataatagtagtaatagtaataacaatacaatgattatggcttatactatacacaagacactgttgatatatt
atttcatttagtattcacagtaactctgtgcctcaagtactattgtaataccctttaagaggaggaaactgaggcacagggccctaaag
taatattccaagatgaagtggctactaactgacagagggcataattcaactcatgatatttggctctagaatacatgctctgaatcatt
atacaataataattcatgaggaaacattttttaaagcctaagttatttgctctgaaataagacataatttggggtgagaaagcttagat
tccatgaagtattacagcatttggtagtctttttgcactccaggtcttatttttactgcttaaacataataaaacatatggttcagtat
gcctttgattttacaataatattcctgttatttttggaagcacagggtgtgggataatgctaattactagtgattagtattgagaggtg
acagcgtgctggcagtcctcacagccctcgctcgctcttggcgcctcctctgcctgggctcccacattggtggcacttgaggagccctt
cagccggccgctgcactgtgggagcccttttctgggctggccaaggccagagccggctccctcagcttgccaggaggtgtggagggaca
gacgcgggcaggaaccgggctgtgcgccgtgcttgagggagttccgggtgggcatgggctccgaggaccccgcactcggagccgccagc
cggccccaccggccgcgggcagtgaggggcttagcacctgggccagcagctgctgtgctcaattcctcgccgggccttagctgccttcc
tgcggggcagggctcgggacctgcagcgcgccatgcctgagcctccccaccttcatgggctcctgtgcggcccgagcctcgccgacgag
cgccgccccctgctccagggcacccagtcccatcgaccacccaagggctgaagagtgcgggcgcacggcaggggactggcaggcagctc
cccctgcagcccaggtgcgggatccactgggtgaagccggctaggctcctgagtttgctggggatgcgaagaacccttatgtctagata
agggattgtaaatacaccaattggcactctgtatctagctcaaggtttgtaaacacaccaatcagcaccctgtgtctagctcagggttt
gtgaatgcaccaatcaacactctatctagctactctggtggggccttggagaacctttatgtctagctcagggattgtaaatacaccaa
tcggcagtctgtatctagctcaaggtttgtaaacacaccaatcagcaccctgtgtctagctcagggtttgtgaatgcaccaatcaacac
tctgtatctagctactctggtggggacgtggagaacctttatgtctagctcagggattgtaaatacaccactcggcagtctgtatctag
ctcaaggtttgtaaacacaccaatcagcaccctgtgtctagctcagggtttgtgaatgcaccaatcaacactctgtatctagctactct
ggtggggacttggagaacctttgtgtggacactctgtatctagctaatctggtggggacgtggagaacctttgtgtctagctcatggat
tgtaaatgcaccaatcagtgccctgtcaaaacagaccactgggctctaccaatcagcaggatgtgggtggggccagataagagaataaa
agcaggctgcccgagccagcagtggcaacccgctcgggtccccttccacactgtggaagctttgttctttcgctctttgcaataaatct
tgctgctgctcactgtttgggtctacactgcctttatgagctgtaacgctcaccgcgaaggtctgcagcttcactcttgaagccagcga
gaccacgaacccaccgggaggaacgaacaactccagaggcgccgccttaagagctggaacgttcactgtgaaggtctgcagcttcactc
ctgagccagcgagaccacgaacccatcagaaggaagaaactccgaacacatccaaacatcagaacgaacaaactccacacacgcagcct
ttaagaactgtaacactcaccacgagggtccccggcttcattcttgaagtcagtgaaaccaagaacccaccaattccggacacagtatg
tcagaaacaatatgagtcactaaatcaatatacttctcaacaatttccaacagcccttgcaattaacttggccatgtgactggttgtga
ctaaaataatgtggagataataatgtgttactccctaaggcagagtgcccttctatcattctctttcccttcctctatgtggcagaaag
taaaagattctgaaatgataaagtcaatcacaggaaggcacctggactcctggcccactgcttggaggagagcactcaggaccatgaac
atctgactgtgacgtagcaataaagaaacccacgtttcatatgaaactgcttaaaattaatggcacaagtcatgtttttgatgttgcac
atttgtctttatttgtggcttgttttgcttccacatcaatccactcaaggcctacattctgctataatgcaatttcaagttctttacag
gccgagaaaaatgaatctgaattcctgacctccaaaagtgatcaagatatttttagttcaggctccaaaattttctcattttcataggt
tttcctcgattgatcattattcatgatttgcaaggaatcattcaatgttttctaaatctattactgcatcctgacacatatgacatttt
aactatgttccagatttttgaatgaagagtgtaaattttaaatgttttcaccacaaaaaataagtatgtgaagtggtggatttgttaat
tagccttatttaaccatttaatattgtacacgtacaccaaagcatcatgttgtaccccatgaatacacacaattattatttgtcaattt
aaaatgaaataataaaaaataacaaaggcattagcctctgcattgcctttaccggtcatcctcacggtgactaacgcaaaaaacgttct
atttcatccttacaaacatccctatctttgatgcctctttgtctagatctctatcccctcctgttttctctacgttatttatatgggta
tcatcaccatcctggacaacatcaggacagatatccctcaccaagccaatgttcctctctatgttggctcaaatgtccttgaactttcc
tttcaccaccctttccacagtcaaaaggatattgtagtttaatgcctcagagttcagcttttaagcttctgacaaattattcttcctct
ttaggttctcctttatggaatcttctgtactgatggccatgtcctttaactactatgtagatatctgctactacctgtattatgcctct
acctttattagcagagttatctgtactgttggcatgacaatcatttgttaatatgacttgcctttcctttttctgctattcttgatcaa
atggctcctctttcttgctcctctcatttctcctgccttcacttggacgtgcttcacgtagtctgtgcttatgactggattaaaaattg
atatggacttatcctaatgttgttcgtcataatatgggttttatggtccattattatttcctatgcattgatctggagaaggcttcaat
ccttttactctttgtggaaaatatctgtaaaccttctggttcactctgctatagcaatttcagtttaggctagtaagcatgaggatgcc
tccttctctgatttttcccacagtctgttggtcacagaataacctgagtgattactgatgaaagagtgagaatgttattgatagtcaca
atgacaaaaaacaaacaactacagtcaaaatgtttctctttttattagtggattatatttcctgacctatatctggcaggactctttag
agaggtagctgaagctgctgttatgaccactagagggaagaagatacctgtggagctaatggtccaagatggtggagccccaagcaagg
aagttgttaaggagcccttttgattgaaggtgggtgcccccaccttacagggacaggacatctggatactcctcccagtttctccagtt
tccctttttcctaatatatctcctgataaaatgtctatactcacttccccatttctaataataaagcaaaggctagttagtaagacatc
accttgcattttgaaaatgccatagactttcaaaattatttcatacatcggtctttctttatttcaagagtccagaaatggcaacatta
cctttgattcaatgtaatggaaagagctctttcaagagacagagaaaagaataatttaatttctttccccacacctccttccctgtctc
ttaccctatcttccttccttctaccctccccatttctctctctcatttctcagaagtatattttgaaaggattcatagcagacagctaa
ggctggttttttctaagtgaagaagtgatattgagaaggtagggttgcatgagccctttcagttttttagtttatatacatctgtattg
ttagaatgttttataatataaataaaattatttctcagttatatactagctatgtaacctgtggatatttccttaagtattacaagcta
tacttaactcacttggaaaactcaaataaatacctgcttcatagttattaataaggattaagtgagataatgcccataagattcctatt
aataacagataaatacatacacacacacacacattgaaaggattcttactttgtgctaggaactataataagttcattgatgcattata
tcattaagttctaatttcaacactagaaggcaggtattatctaaatttcatactggatacctccaaactcataaagataattaaattgc
cttttgtcatatatttattcaaaagggtaaactcaaactatggcttgtctaattttatatatcaccctactgaacatgaccctattgtg
atattttataaaattattctcaagttattatgaggatgttgaaagacagagaggatggggtgctatgccccaaatcagcctcacaatta
agctaagcagctaagagtcttgcagggtagtgtagggaccacagggttaagggggcagtagaattatactcccactttagtttcatttc
aaacaatccatacacacacagccctgagcacttacaaattatactacgctctatactttttgtttaaatgtataaataagtggatgaaa
gaatagatagatagatagacagatagatgatagatagaataaatgcttgccttcatagctgtctccctaccttgttcaaaatgttcctg
tccagaccaaagtaccttgccttcacttaagtaatcaattcctaggttatattctgatgtcaaaggaagtcaaaagatgtgaaaaacaa
tttctgacccacaactcatgctttgtagatgactagatcaaaaaatttcagccatatcttaacagtgagtgaacaggaaatctcctctt
ttccctacatctgagatcccagcttctaagaccttcaattctcactcttgatgcaacagaccttggaagcatacaggagagctgaactt
ggtcaacaaaggagaaaagtttgttggcctccaaaggcacagctcaaacttttcaagccttctctaatcttaaaggtaaacaagggtct
catttctttgagaacttcagggaaaatagacaaggacttgcctggtgcttttggtaggggagcttgcactttccccctttctggaggaa
atatttatccccaggtagttccctttttgcaccagtggttctttgaagagacttccacctgggaacagttaaacagcaactacagggcc
ttgaactgcacactttcagtccggtcctcacagttgaaaagacctaagcttgtgcctgatttaagcctttttggtcataaaacattgaa
ttctaatctccctctcaaccctacagtcacccatttggtatattaaagatgtgttgtctactgtctagtatccctcaagtagtgtcagg
aattagtcatttaaatagtctgcaagccaggagtggtggctcatgtctgtaattccagcacttgagaggtagaagtgggaggactgctt
gagctcaagagtttgatattatcctggacaacatagcaagacctcgtctctacttaaaaaaaaaaaaaaaattagccaggcatgtgatg
tacacctgtagtcccagctactcaggaggccgaaatgggaggatcccttgagctcaggaggtcaaggctgcagtgagacatgatcttgc
cactgcactccagcctggacagcagagtgaaaccttgcctcacgaaacagaatacaaaaacaaacaaacaaaaaactgctccgcaatgc
gcttccttgatgctctaccacataggtctgggtactttgtacacattatctcattgctgttcataattgttagattaattttgtaatat
tgatattattcctagaaagctgaggcctcaagatgataacttttattttctggacttgtaatagctttctcttgtattcaccatgttgt
aactttcttagagtagtaacaatataaagttattgtgagtttttgcaaacacagcaaacacaacgacccatatagacattgatgtgaaa
ttgtctattgtcaatttatgggaaaacaagtatgtactttttctactaagccattgaaacaggaataacagaacaagattgaaagaata
cattttccgaaattacttgagtattatacaaagacaagcacgtggacctgggaggagggttattgtccatgactggtgtgtggagacaa
atgcaggtttataatagatgggatggcatctagcgcaatgactttgccatcacttttagagagctcttggggaccccagtacacaagag
gggacgcagggtatatgtagacatctcattctttttcttagtgtgagaataagaatagccatgacctgagtttatagacaatgagccct
tttctctctcccactcagcagctatgagatggcttgccctgcctctctactaggctgactcactccaaggcccagcaatgggcagggct
ctgtcagggctttgatagcactatctgcagagccagggccgagaaggggtggactccagagactctccctcccattcccgagcagggtt
tgcttatttatgcatttaaatgatatatttattttaaaagaaataacaggagactgcccagccctggctgtgacatggaaactatgtag
aatattttgggttccatttttttttccttctttcagttagaggaaaaggggctcactgcacatacactagacagaaagtcaggagcttt
gaatccaagcctgatcatttccatgtcatactgagaaagtccccacccttctctgagcctcagtttctctttttataagtaggagtctg
gagtaaatgatttccaatggctctcatttcaatacaaaatttccgtttattaaatgcatgagcttctgttactccaagactgagaagga
aattgaacctgagactcattgactggcaagatgtccccagaggctctcattcagcaataaaattctcaccttcacccaggcccactgag
tgtcagatttgcatgcactagttcacgtgtgtaaaaaggaggatgcttctttcctttgtattctcacatacctttaggaaagaacttag
cacccttcccacacagccatcccaataactcatttcagtgactcaacccttgactttataaaagtcttgggcagtatagagcagagatt
aagagtacagatgctggagccagaccacctgagtgattagtgactcagtttctcttagtagttgtatgactcagtttcttcatctgtaa
aatggagggttttttaattagtttgtttttgagaaagggtctcactctgtcacccaaatgggagtgtagtggcaaaatctcggctcact
gcaacttgcacttcccaggctcaagcggtcctcccacctcaacatcctgagtagctggaaccacaggtacacaccaccatacctcgcta
attttttgtatttttggtagagatggggtttcacatgttacacaggatggtctcagactccggagctcaagcaatctgcccacctcagc
cttccaaagtgctgggattataagcatgattacaggagttttaacaggctcataagattgttctgcagcccgagtgagttaatacatgc
aaagagtttaaagcagtgacttataaatgctaactactctagaaatgtttgctagtattttttgtttaactgcaatcattcttgctgca
ggtgaaaactagtgttctgtactttatgcccattcatctttaactgtaataataaaaataactgacatttattgaaggctatcagagac
tgtaattagtgctttgcataattaatcatatttaatactcttggattctttcaggtagatactattattatccccattttactacagtt
aaaaaaactacctctcaacttgctcaagcatacactctcacacacacaaacataaactactagcaaatagtagaattgagatttggtcc
taattatgtctttgctcactatccaataaatatttattgacatgtacttcttggcagtctgtatgctggatgctggggatacaaagatg
tttaaatttaagctccagtctctgcttccaaaggcctcccaggccaagttatccattcagaaagcattttttactctttgcattccact
gtttttcctaagtgactaaaaaattacactttattcgtctgtgtcctgctctgggatgatagtctgactttcctaacctgagcctaaca
tccctgacatcaggaaagactacaccatgtggagaaggggtggtggttttgattgctgctgtcttcagttagatggttaactttgtgaa
gttgaaaactgtggctctctggttgactgttagagttctggcacttgtcactatgcctattatttaacaaatgcatgaatgcttcagaa
tatgggaatattatcttctggaatagggaatcaagttatattatgtaacccaggattagaagattcttctgtgtgtaagaatttcataa
acattaagctgtctagcaaaagcaagggcttggaaaatctgtgagctcctcaccatatagaaagcttttaacccatcattgaataaatc
cctataggggatttctaccctgagcaaaaggctggtcttgattaattcccaaactcatatagctctgagaaagtctatgctgttaacgt
tttcttgtctgctaccccatcatatgcacaacaataaatgcaggcctaggcatgactgaaggctctctcataattcttggttgcatgaa
tcagattatcaacagaaatgttgagacaaactatggggaagcagggtatgaaagagctctgaatgaaatggaaaccgcaatgcttcctg
cccattcagggctccagcatgtagaaatctggggctttgtgaagactggcttaaaatcagaagccccattggataagagtagggaagaa
cctagagcctacgctgagcaggtttccttcatgtgacagggagcctcctgccccgaacttccagggatcctctcttaagtgtttcctgc
tggaatctcctcacttctatctggaaatggtttctccacagtccagcccctggctagttgaaagagttacccatgcagaggccctccta
gcatccagagactagtgcttagattcctactttcagcgttggacaacctggatccacttgcccagtgttcttccttagttcctaccttc
gaccttgatcctcctttatcttcctgaaccctgctgagatgatctatgtggggagaatggcttctttgagaaacatcttcttcgttagt
ggcctgcccctcattcccactttaatatccagaatcactataagaagaatataataagaggaataactcttattataggtaagggaaaa
ttaagaggcatacgtgatgggatgagtaagagaggagagggaaggattaatggacgataaaatctactactatttgttgagacctttta
tagtctaatcaattttgctattgttttccatcctcacgctaactccataaaaaaacactattattatctttattttgccatgacaagac
tgagctcagaagagtcaagcatttgcctaaggtcggacatgtcagaggcagtgccagacctatgtgagactctgcagctactgctcatg
ggccctgtgctgcactgatgaggaggatcagatggatggggcaatgaagcaaaggaatcattctgtggataaaggagacagccatgaag
aagtctatgactgtaaatttgggagcaggagtctctaaggacttggatttcaaggaattttgactcagcaaacacaagaccctcacggt
gactttgcgagctggtgtgccagatgtgtctatcagaggttccagggagggtggggtggggtcagggctggccaccagctatcagggcc
cagatgggttataggctggcaggctcagataggtggttaggtcaggttggtggtgctgggtggagtccatgactcccaggagccaggag
agatagaccatgagtagagggcagacatgggaaaggtgggggaggcacagcatagcagcatttttcattctactactacatgggactgc
tcccctatacccccagctaggggcaagtgccttgactcctatgttttcaggatcatcatctataaagtaagagtaataattgtgtctat
ctcatagggttattatgaggatcaaaggagatgcacactctctggaccagtggcctaacagttcaggacagagctatgggcttcctatg
tatgggtcagtggtctcaatgtagcaggcaagttccagaagatagcatcaaccactgttagagatatactgccagtctcagagcctgat
gttaatttagcaatgggctgggaccctcctccagtagaaccttctaaccagctgctgcagtcaaagtcgaatgcagctggttagacttt
ttttaatgaaagcttagctttcattaaagattaagctcctaagcagggcacagatgaaattgtctaacagcaactttgccatctaaaaa
aatctgacttcactggaaacatggaagcccaaggttctgaacatgagaaatttttaggaatctgcacaggagttgagagggaaacaaga
tggtgaagggactagaaaccacatgagagacacgaggaaatagtgtagatttaggctggaggtaaatgaaagagaagtgggaattaata
cttactgaaatctttctatatgtcaggtgccattttatgatatttaataatctcattacatatggtaattctgtgagatatgtattatt
gaacatactataattaatactaatgataagtaacacctcttgagtacttagtatatgctagaatcaaatttaagtttatcatatgaggc
cgggcacggtggctcatatatgggattacatgcctgtaatcccagcactttgggaggccaaggcaattggatcacctgaggtcaggagt
tccagaccagcctggccaacatggtgaaaccccttctctactaaaaaatacaaaaaatcagccaggtgtggtggcacgcgtctataatc
ccagctactcaggaggctgaggcaggagaatcacttgaacccaggaggtggaggttgcagtgagctaagattgcaccactgcactccag
cctaggcgacagagtgagactccatctcaaaaaaaaaaaaagaagtttattatatgaattaacttagttttactcacaccaatactcag
aagtagattattacctcatttattgatgaggagcccaatgtacttgtagtgtagatcaacttattgaaagcacaagctaataagtagac
aattagtaattagaagtcagatggtctgagctctcctactgtctacattacatgagctcttattaactggggactcgaaaatcaaagac
atgaaataatttgtccaagcttacagaaccaccaagtagtaaggctaggatgtagacccagttctgctacctctgaagacagtgttttt
tccacagcaaaacacaaactcagatattgtggatgcgagaaattagaagtagatattcctgccctgtggcccttgcttcttacttttac
ttcttgtcgattggaagttgtggtccaagccacagttgcagaccatacttcctcaaccataattgcatttcttcaggaaagtttgaggg
agaaaaaggtaaagaaaaatttagaaacaacttcagaataaagagattttctcttgggttacagagattgtcatatgacaaattataag
cagacacttgagaaaactgaaggcccatgcctgcccaaattaccctttgaccccttggtcaagctgcaactttggttaaagggagtgtt
tatgtgttatagtgttcatttactcttctggtctaacccattggctccgtcttcatcctgcagtgacctcagtgcctcagaaacataca
tatgtttgtctagtttaagtttgtgtgaaattctaactagcgtcaagaactgagggccctaaactatgctaggaatagtgctgtggtgc
tgtgataggtacacaagaaatgagaagaaactgcagattctctgcatctccctttgccgggtctgacaacaaagtttccccaaatttta
ccaatgcaagccatttctccatatgctaactactttaaaatcatttggggcttcacattgtctttctcatctgtaaaaagaatggaaga
actcattcctacagaactccctatgtcttccctgatgggctagagttcctctttctcaaaaattagccattattgtatttccttctaag
ccaaagctcagaggtcttgtattgcccagtgacatgcacactggtcaaaagtaggctaagtagaagggtactttcacaggaacagagag
caaaagaggtgggtgaatgagagggtaagtgagaaaagacaaatgagaagttacaacatgatggcttgttgtctaaatatctcctaggg
aattattgtgagaggtctgaatagtgttgtaaaataagctgaatctgctgccaacattaacagtcaagaaatacctccgaataactgta
cctccaattattctttaaggtagcatgcaactgtaatagttgcatgtatatatttatcataatactgtaacagaaaacacttactgaat
atatactgtgtccctagttctttacacaataaactaatctcatcctcataattctattagctaatacatattatcatcctatatttcag
agacttcaagaagttaagcaacttgctcaagatcatctaagaagtaggtggtatttctgggctcatttggcccctcctaatctctcatg
gcaacatggctgcctaaagtgttgattgccttaattcatcagggatgggctcatactcactgcagaccttaactggcatcctcttttct
tatgtgatctgcctgaccctagtagacttatgaaatttctgatgagaaaggagagaggagaaaggcagagctgactgtgatgagtgatg
aaggtgccttctcatctgggtaccagtggggcctctaagactaagtcactctgtctcactgtgtcttagccagttccttacagcttgcc
ctgatgggagatagagaatgggtatcctccaacaaaaaaataaattttcatttctcaaggtccaacttatgttttcttaatttttaaaa
aaatcttgaccattctccactctctaaaataatccacagtgagagaaacattcttttcccccatcccataaatacctctattaaatatg
gaaaatctgggcatggtgtctcacacctgtaatcccagcactttgggaggctgaggtgggtggactgcttggagctcaggagttcaaga
ccatcttggacaacatggtgataccctgcctctacaaaaagtacaaaaattagcctggcatggtggtgtgcacctgtaatcccagctat
tagggtggctgaggcaggagaattgcttgaacccgggaggcggaggttgcagtgagctgagatcgtgccactgcactccagcctggggg
acagagcacattataattaactgttattttttacttggactcttgtggggaataagatacatgttttattcttatttatgattcaagca
ctgaaaatagtgtttagcatccagcaggtgcttcaaaaccatttgctgaatgattactatactttttacaagctcagctccctctatcc
cttccagcatcctcatctctgattaaataagcttcagtttttccttagttcctgttacatttctgtgtgtctccattagtgacctccca
tagtccaagcatgagcagttctggccaggcccctgtcggggtcagtgccccacccccgccttctggttctgtgtaaccttctaagcaaa
ccttctggctcaagcacagcaatgctgagtcatgatgagtcatgctgaggcttagggtgtgtgcccagatgttctcagcctagagtgat
gactcctatctgggtccccagcaggatgcttacagggcagatggcaaaaaaaaggagaagctgaccacctgactaaaactccacctcaa
acggcatcataaagaaaatggatgcctgagacagaatgtgacatattctagaatatattatttcctgaatatatatatatatatacaca
tatacgtatatatatatatatatatatatttgttgttatcaattgccatagaatgattagttattgtgaatcaaatatttatcttgcag
gtggcctctatacctagaagcggcagaatcaggctttattaatacatgtgtatagatttttaggatctatacacatgtattaatatgaa
acaaggatatggaagaggaaggcatgaaaacaggaaaagaaaacaaaccttgtttgccattttaaggcacccctggacagctaggtggc
aaaaggcctgtgctgttagaggacacatgctcacatacggggtcagatctgacttggggtgctactgggaagctctcatcttaaggata
catctcaggccagtcttggtgcattaggaagatgtaggcaactctgatcctgagaggaaagaaacattcctccaggagagctaaaaggg
ttcacctgtgtgggtaactgtgaaggactacaagaggatgaaaaacaatgacagacagacataatgcttgtgggagaaaaaacaggagg
tcaaggggatagagaaggcttccagaagaatggctttgaagctggcttctgtaggagttcacagtggcaaagatgtttcagaaatgtga
catgacttaaggaactatacaaaaaggaacaaatttaaggagaggcagataaattagttcaacagacatgcaaggaattttcagatgaa
tgttatgtctccactgagcttcttgaggttagcagctgtgagggttttgcaggcccaggacccattacaggacctcacgtatacttgac
actgttttttgtattcatttgtgaatgaatgacctcttgtcagtctactcggtttcgctgtgaatgaatgatgtcttgtcagcctactt
ggtttcgctaagagcacagagagaagatttagtgatgctatgtaaaaacttcctttttggttcaagtgtatgtttgtgatagaaatgaa
gacaggctacatgatgcatatctaacataaacacaaacattaagaaaggaaatcaacctgaagagtatttatacagataacaaaataca
gagagtgagttaaatgtgtaataactgtggcacaggctggaatatgagccatttaaatcacaaattaattagaaaaaaaacagtgggga
aaaaattccatggatgggtctagaaagactagcattgttttaggttgagtggcagtgtttaaagggtgatatcagactaaacttgaaat
atgtggctaaataactagaatactctttattttttcgtatcatgaatagcagatatagcttgatggccccatgcttggtttaacatcct
tgctgttcctgacatgaaatccttaatttttgacaaaggggctattcattttcattttatattgggcctagaaattatgtagatggtcc
tgaggaaaagtttatagcttgtctatttctctctctaacatagttgtcagcacaatgcctaggctataggaagtactcaaagcttgtta
aattgaattctatccttcttattcaattctacacatggaggaaaaactcatcagggatggaggcacgcctctaaggaaggcaggtgtgg
ctctgcagtgtgattgggtacttgcaggacgaagggtggggtgggagtggctaaccttccattcctagtgcagaggtcacagcctaaac
atcaaattccttgaggtgcggtggctcactcctgtaatcacagcagtttgggacgccaaggtgggcagatcacttgaggtcaggagttg
gacaccagcccagccaacatagtgaaacctggtctctgcttaaaaatataaaaattagctggacgtggtgacgggagcctgtaatccaa
ctacttgggaggctgaggcaggagaatcgcttgaaccggggaggtggagtttgcactgagcagagatcatgccattgcactccagcctc
cagagcgagactctgtctaaagaaaaacgaaaacaaacaaacaaacaaacaaacaaaacccatcaaattccctgaccgaacagaattct
gtctgattgttctctgacttatctaccattttccctccttaaagaaactgtgaacttccttcagctagaggggcctggctcagaagcct
ctggtcagcatccaagaaatacttgatgtcactttggctaaaggtatgatgtgtagacaagctccagagatggtttctcatttccatat
ccacccacccagctttccaattttaaagccaattctgaggtagagactgtgatgaacaaacaccttgacaaaattcaacccaaagactc
actttgcctagcttcaaaatccttactctgacatatactcacagccagaaattagcatgcactagagtgtgcatgagtgcaacacacac
acacaccaattccatattctctgtcagaaaatcctgttggtttttcgtgaaaggatgttttcagaggctgaccccttgccttcacctcc
aatgctaccactctggtctaagtcactgtcaccaccacctaaattatagctgttgactcataacaatcttcctgcttctaccactgccc
cactacaatttcttcccaatatactatccaaattagtcttttcaaaatgtaagtcatatatggtcacctctttgttcaaagtcttctga
tagtttcctatatcatttataataaaaccaaatccttacaattctctacaatagttgttcatgcatatattatgtttattacagataca
tatatatagctctcatataaataaatatatatatttatgtgtatgtgtgtagagtgttttttcttacaactctatgatgtaggtattat
tagtgtcccaaattttataatttaggacttctatgatctcatcttttattctccccttcaccgaatctcatcctacattggccttattg
atattccttgaaaattctaagcatcttacatctttagggtatttacatttgccattccctatgccctaaatatttaatcatagtttcat
ataaatgggttcctcatcatctatgggtactctctcaggtgttaactttatagtgaggactttcctgccatactacttaaagtagcgat
accctttcaccctgtcctaatcacactctggccttcatttcagttttttttttttctccatagcacctaatctcattggtatataacat
gtttcatttgcttatttaatgtcaagctctttccactatcaagtccatgaaaacaggaactttattcctctattctgtttttgtgctgt
attcttagcaattttacaattttgaatgaatgaatgagcagtcaaacacatatacaactataattaaaaggatgtatgctgacacatcc
actgctatgcacacacaaagaaatcagtggagtagagctggaagtgctaagcctgcatagagctagttagccctccgcaggcagagcct
tgatgggattactgagttctagaattggactcatttgttttgtaggctgagatttgctcttgaaaacttgttctgaccaaaataaaagg
ctcaaaagatgaatatcgaaaccagggtgttttttacactggaatttataactagagcactcatgtttatgtaagcaattaattgtttc
atcagtcaggtaaaagtaaagaaaaactgtgccaaggcaggtagcctaatgcaatatgccactaaagtaaacattatttcataggtgtc
agatatggcttattcatccatcttcatgggaaggatggccttggcctggacatcagtgttatgtgaggttcaaaacacctctaggctat
aaggcaacagagctccttttttttttttctgtgctttcctggctgtccaaatctctaatgataagcatacttctattcaatgagaatat
tctgtaagattatagttaagaattgtgggagccattccgtctcttatagttaaatttgagcttcttttatgatcactgtttttttaata
tgctttaagttctggggtacatgtgccatggtggtttgctgcacccatcaacccgtcatctacattaggtatttctcctaatgctatcc
ttcccctagccccccacccccaacaggccccagtgtgtgatgttcccctccctgtgtccatggatcactggtttttttttgtttttttt
ttttttttaaagtctcagttaaatttttggaatgtaatttattttcctggtatcctaggacttgcaagttatctggtcactttagccct
cacgttttgatgataatcacatatttgtaaacacaacacacacacacacacacacacacatatatatatatataaaacatatatataca
taaacacacataacatatttatcgggcatttctgagcaactaatcatgcaggactctcaaacactaacctatagccttttctatgtatc
tacttgtgtagaaaccaagcgtggggactgagaaggcaatagcaggagcattctgactctcactgcctttagctaggcccctccctcat
cacagctcagcatagtcctgagctcttatctatatccacacacagtttctgacgctgcccagctatcaccatcccaagtctaaagaaaa
aaataatgggtttgcccatctctgttgattagaaaacaaaacaaaataaaataagcccctaagctcccagaaaacatgactaaaccagc
aagaagaagaaaatacaataggtatatgaggagactggtgacactagtgtctgaatgaggcttgagtacagaaaagaggctctagcagc
atagtggtttagaggagatgtttctttccttcacagatgccttagcctcaataagcttgcggttgtggaagtttactttcagaacaaac
tcctgtggggctagaattattgatggctaaaagaagcccgggggagggaaaaatcattcagcatcctcacccttagtgacacaaaacag
agggggcctggttttccatatttcctcatgatggatgatctcgttaatgaaggtggtctgacgagatcattgcttcttccatttaagcc
ttgctcacttgccaatcctcagttttaaccttctccagagaaatacacattttttattcaggaaacatactatgttatagtttcaatac
taaataatcaaagtactgaagatagcatgcataggcaagaaaaagtccttagctttatgttgctgttgtttcagaatttaaaaaagatc
accaagtcaaggacttctcagttctagcactagaggtggaatcttagcatataatcagaggtttttcaaaatttctagacataagattc
aaagccctgcacttaaaatagtctcatttgaattaactctttatataaattgaaagcacattctgaactacttcagagtattgttttat
ttctatgttcttagttcataaatacattaggcaatgcaatttaattaaaaaaacccaagaatttcttagaattttaatcatgaaaataa
atgaaggcatctttacttactcaaggtcccaaaaggtcaaagaaaccaggaaagtaaagctatatttcagcggaaaatgggatatttat
gagttttctaagttgacagactcaagttttaaccttcagtgcccatcatgtaggaaagtgtggcataactggctgattctggctttcta
ctcctttttcccattaaagatccctcctgcttaattaacattcacaagtaactctggttgtactttaggcacagtggctcccgaggtca
gtcacacaataggatgtctgtgctccaagttgccagagagagagattactcttgagaatgagcctcagccctggctcaaactcacctgc
aaacttcgtgagagatgaggcagaggtacactacgaaagcaacagttagaagctaaatgatgagaacacatggactcatagagggaaac
aacgcatactggggcctatcagagggtggagggtgagagaaggagaggatcaggaaaaatcactaatggatgctaagcgtaatacctga
gtgatgagatcatctatacaacaaacccccttgacattcatttatctatgtaacaaacctgcacatcctgtacatgtacccctgaactt
aaaataaaagttgaaaacaagaaagcaacagtttgaacacttgttatggtctattctctcattctttacaattacactagaaaatagcc
acaggcttcctgcaaggcagccacagaatttatgacttgtgatatccaagtcattcctggataatgcaaaatctaacacaaaatctagt
agaatcatttgcttacatctatttttgttctgagaatatagatttagatacataatggaagcagaataatttaaaatctggctaattta
gaatcctaagcagctcttttcctatcagtggtttacaagccttgtttatatttttcctattttaaaaataaaaataaagtaagttattt
gtggtaaagaatattcattaaagtatttatttcttagataataccatgaaaaacattcagtgaagtgaagggcctactttacttaacaa
gaatctaatttatataatttttcatactaatagcatctaagaacagtacaatatttgactcttcaggttaaacatatgtcataaattag
ccagaaagatttaagaaaatattggatgtttccttgtttaaattaggcatcttacagtttttagaatcctgcatagaacttaagaaatt
acaaatgctaaagcaaacccaaacaggcaggaattaatcttcatcgaatttgggtgtttctttctaaaagtcctttatacttaaatgtc
ttaagacatacatagattttattttactaattttaattatatagacaataaatgaatattcttactgattactttttctgactgtctaa
tctttctgatctatcctggatggccataacacttatctctctgaactttgggcttttaatataggaaagaaaagcaataatccattttt
catggtatctcatatgataaacaaataaaatgcttaaaaatgagcaggtgaagcaatttatcttgaaccaacaagcatcgaagcaataa
tgagactgcccgcagcctacctgacttctgagtcaggatttataagccttgttactgagacacaaacctgggcctttcaatgctataac
ctttcttgaagctcctccctaccacctttagccataaggaaacatggaatgggtcagatccctggatgcaagccaggtctggaaccata
ggcagtaaggagagaagaaaatgtgggctctgcaactggctccgagggagcaggagaggatcaaccccatactctgaatctaagagaag
actggtgtccatactctgaatgggaagaatgatgggattacccatagggcttgttttagggagaaacctgttctccaaactcttggcct
tgagatacctggtccttattccttggactttggcaatgtctgaccctcacattcaagttctgaggaagggccactgccttcatactgtg
gatctgtagcaaattccccctgaaaacccagagctgtatcttaattggttaaaaaaaattatattatctcaacgactgttcttctctga
gtagccaagctcagcttggttcaagctacaagcagctgagctgctttttgtctagtcattgttcttttatttcagtggatcaaatacgt
tctttccaaacctaggatcttgtcttcctaggctatatattttgtcccaggaagtcttaatctggggtccacagaacactagggggctg
gtgaagtttatagaaaaaaaatctgtatttttacttacatgtaactgaaatttagcattttcttctactttgaatgcaaaggacaaact
agaatgacatcatcagtacctattgcatagttataaagagaaaccacagatattttcatactacaccataggtattgcagatctttttg
tttttgtttttgtttgagatggagtttcgctcttattgcccaggctggagtgcagtggcatgatttcggctcactgcaacctccccttc
ctgcattcaagcaattctcctgccttggcctcctgagtagctggggattacaggcacctgccaccatgccagtctaatttttgtatttt
tagtagagatggggtttcgccatgttggccaggctggtcttgaactcctgacctcagatgatctgcccgccttggcctcctgaagtgct
gggattataggtgtgagccaccacgcctggcccattgcagatatttttaattcacatttatctgcatcactacttggatcttaaggtag
ctgtagacccaatcctagatctaatgctttcataaagaagcaaatataataaatactataccacaaatgtaatgtttgatgtctgataa
tgatatttcagtgtaattaaacttagcactcctatgtatattatttgatgcaataaaaacatatttttttagcacttacagtctgccaa
actggcctgtgacacaaaaaaagtttaggaattcctggttttgtctgtgttagccaatggttagaatatatgctcagaaagataccatt
ggttaatagctaaaagaaaatggagtagaaattcagtggcctggaataataacaatttgggcagtcattaagtcaggtgaagacttctg
gaatcatgggagaaaagcaagggagacattcttacttgccacaagtgtttttttttttttttttttttatcacaaacataagaaaatat
aataaataacaaagtcaggttatagaagagagaaacgctcttagtaaacttggaatatggaatccccaaaggcacttgacttgggagac
aggagccatactgctaagtgaaaaagacgaagaacctctagggcctgaacatacaggaaattgtaggaacagaaattcctagatctggt
ggggcaaggggagccataggagaaagaaatggtagaaatggatggagacggaggcagaggtgggcagatcatgaggtcaagagatcgag
accatcctggcaaacatggtgaaatcccgtctctactaaaaataaaaaaattagctgggcatggtggcatgcgcctgtagtcccagctg
ctcgggaggctgaggcaggagaatcgtttgaacccaggaggcgaaggttgcagtgagctgagatagtgccattgcactccagtctggca
acagagtgagactccgtctcaaaaaaaaaaaaaaaagaaagaaagaaaagaaaaagaaaaaagaaaaaataaatggatgtagaacaagc
cagaaggaggaactgggctggggcaatgagattatggtgatgtaagggacttttatagaattaacaatgctggaatttgtggaactctg
cttctattattcccccaatcattacttctgtcacattgatagttaaataatttctgtgaatttattccttgattctaaaatatgaggat
aatgacaatggtattataagggcagattaagtgatatagcatgagcaatattcttcaggcacatggatcgaattgaatacactgtaaat
cccaacttccagtttcagctctaccaagtaaagagctagcaagtcatcaaaatggggacatacagaaaaaaaaaaggacactagaggaa
taatataccctgactcctagcctgattaatatatcgattcacttttttctctgtttgatgacaaattctggctttaaataattttagga
ttttaggcttctcagctcccttcccagtgagaagtataagcaggacagacaggcaagcaagaagagagccccaggcaatactcacaaag
tagccaatgtcccctgtggtcatagagaaatgaaaagagagaggattctctggaagcactggatgtaatcttttctgtctgtcctctct
agggaatcaccccaaggtactgtactttgggattaaggctttagtcccactgtggactacttgctattctgttcagtttctagaaggaa
ctatgtacggtttttgtctccctagagaaactaaggtacagaagttttgtttacaatgcactccttaagagagctagaactgggtgaga
ttctgttttaacagctttattttcttttccttggccctgtttttgtcactgtcaccacctttaaggcaaatgttaaatgcgctttggct
gaaactttttttcctattttgagatttgctcctttatatgaggctttcttggaaaaggagaatgggagagatggatatcattttggaag
atgatgaagagggtaaaaaaggggacaaatggaaatttgtgttgcagatagatgaggagccaacaaaaaagagcctcaggatccagcac
acattatcacaaacttagtgtccatccatcactgctgaccctctccggacctgactccacccctgagggacacaggtcagccttgacca
atgacttttaagtaccatggagaacagggggccagaacttcggcagtaaagaataaaaggccagacagagaggcagcagcacatatctg
cttccgacacagctgcaatcactagcaagctctcaggcctggcatcatggtgcattttactgctgaggagaaggctgccgtcactagcc
tgtggagcaagatgaatgtggaagaggctggaggtgaagccttgggcaggtaagcattggttctcaatgcatgggaatgaagggtgaat
attaccctagcaagttgattgggaaagtcctcaagattttttgcatctctaattttgtatctgatatggtgtcatttcatagactcctc
gttgtttacccctggacccagagattttttgacagctttggaaacctgtcgtctccctctgccatcctgggcaaccccaaggtcaaggc
ccatggcaagaaggtgctgacttcctttggagatgctattaaaaacatggacaacctcaagcccgcctttgctaagctgagtgagctgc
actgtgacaagctgcatgtggatcctgagaacttcaaggtgagttcaggtgctggtgatgtgattttttggctttatattttgacatta
attgaagctcataatcttattggaaagaccaacaaagatctcagaaatcatgggtcgagcttgatgttagaacagcagacttctagtga
gcataaccaaaacttacatgattcagaactagtgacagtaaaggactactaacagcctgaattggcttaacttttcaggaaatcttgcc
agaacttgatgtgtttatcccagagaattgtattatagaattgtagacttgtgaaagaagaatgaaatttggcttttggtagatgaaag
tccatttcaaggaaatagaaatgccttattttatgtgggtcatgataattgaggtttagaaagagatttttgcaaaaaaaataaaagat
ttgctcaaagaaaaataagacacattttctaaaatatgttaaatttcccatcagtattgtgaccaagtgaaggcttgtttccgaatttg
ttggggattttaaactcccgctgagaactcttgcagcactcacattctacatttacaaaaattagacaattgcttaaagaaaaacaggg
agagagggaacccaataatactggtaaaatggggaagggggtgagggtgtaggtaggtagaatgttgaatgtagggctcatagaataaa
attgaacctaagctcatctgaattttttgggtgggcacaaaccttggaacagtttgaggtcagggttgtctaggaatgtaggtataaag
ccgtttttgtttgtttgtttgttttttcatcaagttgttttcggaaacttctactcaacatgcctgtgtgttattttgtcttttgccta
acagctcctgggtaacgtgatggtgattattctggctactcactttggcaaggagttcacccctgaagtgcaggctgcctggcagaagc
tggtgtctgctgtcgccattgccctggcccataagtaccactgagttctcttccagtttgcaggtgttcctgtgaccctgacaccctcc
ttctgcacatggggactgggcttggccttgagagaaagccttctgtttaataaagtacattttcttcagtaatcaaaaattgcaatttt
atcttctccatcttttactcttgtgttaaaaggaaaaagtgttcatgggctgagggatggagagaaacataggaagaaccaagagcttc
cttaagaaatgtatgggggcttgtaaaattaatgtggatgttatgggagaattccaggattccaaggaggatgatatgatggagaaaaa
tctttatcggggtgggaaaatggttaattaagtggacagagactcctaggcagtttttactgcaccggggaaagaaggagctgttagtg
gtacctgagaaagcagatttgtggtacatgtcacttttcattaaaaacaaaaacaaaacaaaacaaaacttcatagatatccaagatat
aggctagaattactattttaatttactcttatttacattttgaagtagctagcttgtcacatgttttatgaaattgatttggagataag
atgagtgtgtatcaacaatagcctgctctttccatgaaggattccattatttcatgggttagctgaagctaagacacatgatatcattg
tgcattatcttctgatagaatgtaacatgcactaaaataaagttagagttaggacctgagtgggaaagtttttggagagtgtgatgaag
actttccgtgggagatagaatactaataaaggcttaaattctaaaaccagcaagctagggcttcgtgacttgcatgaaactggctctct
ggaagtagaagggagagtaagacatacgtagaggactaggaaagaccagatagtacagggcctggctacaaaaatacaagcttttacta
tgctattgcaatactaaacgataagcattaggatgttaagtgactcaggaaataagattttgggaaaaagtaatctgcttatgtgcaca
aaatggattcaagtttgcagataaaataaaatatggatgatgattcaaggggacagatacaatggttcaaacccaagaggagcagtgag
tctgtggaatttgaaggatggacaaaggtggggtgagaaagacatagtattcgactgactgtgggagatgagaaggaagaaggaggtga
taaatgactgaaagctcccagactggtgaagataacaggaggaaaccatgcactgacctggtgactctcatgtgtgaagggtagaggga
tattaacagatttactttttaggaagtgctagattggtcagggagttttgaccttcaggtcttgtgtctttcatatcaaggaacctttg
cattttccaagttagagtgccatattttggcaaatataactttattagtaattttatagtgctctcacattgatcagactttttcctgt
gaattacttttgaatttggctgtatatatccagaatatgggagagagacaaataattattgtagttgcaggctatcaacaatactggtc
tctctgagccttataacctttcaatatgcccataaacagagtaaacagggattattcatggcactaaatattttcacctagtcagtcaa
caaatgggagcaatgtgcattttttgatacatatttttatatatttatggggtacatgtgatacttacatgcctagaacatgtgatgat
taagtctagatatttaggatatccattgctttgagcatttatcatttctatgtattgagaaaatttcaaatcctcatttctagccattt
tgaaatatataataaatagtaattaactatagtcaccctactcaaatatcaaacattatggcttaatccttctatccaactgtgtttgt
acctattaaccaacatctcttaaatcccctcccatacacactcacactttttccagcctctgataactatcattctactctctaccacc
atgagacccacttttttagctcccacagatgaataaaaacatgtgatatttgactttctgtatctggcttattttattatctatctctt
tggcataccaagagtttgtttttgttctgcttcagggctttcaattaacataatgacctctggttccatccatgttgctacaaatgaca
agatttcattctttttcatggcaaaatagtactgtgcaaaaatacaattttttaatccgttcatctgttgatagacacttaggttgatc
ccaaaccttaactattgtgaatagtgcttcaataaacatgagtgtaatgtgtccattggatatactgatttcctttcttttggataaat
aaccactagtgagattgctggattgtatgatagttctgtttttagtttactgagaaatcttcatactgttttccataatggttgtacta
ttttacattcccaccaacagtgtgtaagaaagagttcccttttctccatatcctcacaaggatctgttattttttgtcttttttgttaa
tagccgttttaactagagtaagtagatatctcattgtagttttgatttgcatttccctgatcattagtgatgttgagaattttttcata
tgtttgttggtcatttgtatatctttttctgagaattgtctgttcatgtccttagcctactttttattgggattgtttgttattttctt
gataatctatttgtgttcattttagagcctggatattattcttttgtcagatgtatagattgtgaagattttctcccactctgtgggtt
gtctgtttattctgcagactcttccttttgccatgcaaaagctctttagtttaatttagtcccagatattttctttgtttttatgtatt
tgcatttgtgttcttggtcatgaaatcctttcctaagccaatgtgtagaagggtttttccgatgttattttctagaattgttacagttt
cagggcttagatttaagtccttgatccatcttgagttgatttttgtataaggtgagagatgaagatccagtttcattctcctacatgta
gcttgccagctatccccgcaccatttgttgaatagggtgccctttccccactttatgtttttgtttgctttgtcaaagatcagttggat
gtaagtatttgagtttatttctgggttctctattctgttccattggtcgatgtgcctatttgtacaccagcatcatgctgttttggtga
ctatggccttattgtatagtttgaaatgaggtaatgtaatgccttcagatttgttcttttttttagacttgcttgtttattgggctctt
ttttggttccataagaattttaggattgttttttctagttctgtgaagactaatggtggtattttgatgggaattgcaatgaatttgta
ggttgcttctggcattatggccattttcacaatattgattctacccatctatgagaatggcatgtgtttccatttgtttgtgtcttata
tgattactttcagccgtgttttgtagttttccttgtagatgtctttcacctccttggttaggtatatattcctaagtttttgttttgtt
ttgttttgttttttgcagctattgtaaaaggggttgagttcttgattttattctcagcttggtcattgctggtatgtaagaaagcaact
cattggtgtacgttaattttgtatccagaaactttgctgaattattttatcagttctagggggttttggaggagtctttagagttttct
acatacacaatcatatcatcagcaaacagtgacagtttgactttctctttaacaatttggatgtgctttacttgtttctcttgtctgat
tgctcttgctaggacttccagtaatatgttaaagagaagtggtgagagtgggtatccttgtctcattccagttttcagacagaatgctt
ttaactttttcccattcaatataatgttggctgtgtgtttaccatagctggcttttattacattgaggtatgtcctttgtaaaccgatt
ttgctgagttttagtcataaagtgatgttgaattttgttgaatgcagtttctgtggctattgagataatcacatgatttttgtttccaa
ttctctttatgttgtgtatcacacttattgacttgcgtatgttaaaccatccgtgcatccctcgcatgaaacccacttgatcatgggtt
ttgatatgctgtcggatgctattagctagtattttgtcaaggatgttggcatctatgttcatcagggatattgatctgtagtgtttttt
ttttttggttatgttctttcccagttttggtattaaggtgatactggcttcatagaatgatttagggaggattctctctttctctatct
tgtagaatactgtcaataggattggtatcaattcttctttgaatgtctggtagaattcagctgtgaatctatctggtcctggacttttt
tgttgttggtaaatttttattatcatttcagtcttgctgcttattactggtctgttcagggtatctaattcttcctgacttaagctaga
gccctgtatctttccaggaattcgaacgtctcctttaggttttctagtttatgcatgtaaaggtgttcatagtagccttgaataatctt
ttgtatttctgtggtatcagtaatagtatctcctgttttgtttctaattgagtttatttgcacttctctcctcttttcttggttaatct
tgctaatggtctatcagttttatttatcttttcaaagaaccagctttttatttcatttagcttttgtatttttttgcagttgttttaat
ttcatttagttctcctcttatcttagttattccctttcttttgctgggttttggttctgtttgtttttgtttctctagtttcttgtggt
gtgaccttatattgtctgtctgtcctctttcagactctttgacatcgacatttagggctgtgaactttccttttagcaccatctttgct
gtatcctagaggttttgataggttgtgtcactattgtcggtcagttcaagtaattttgttgttcttattatactttaagttctgggata
catgtgcagaatgtgcaggtttgttacataggtatagatgtgccatggtggtttgctgcacccatcaacctgtcatctacattaggtat
ttcttttaatgttatccctctcctaaccccctcaccccccgacaggccctggtgtgtgatgttcccctccctgtgtccatgtgttctca
ttgttcaactcccacttatgagtgagaacgtgtggtgtttggtttctctgttcctgtgttagtttgctcagaatgatggtttccacctt
catccatgtccctgcaaagacatgaactcatcatttttatggctgcatagtattccatggtgtatatgtgccacattttctttatccat
tatatcgctgatggccatttgggttggttccaagtctttgctattgtgaatagtgccacaataaacatacgtgtgcacgtgtctttata
gtagaatgatttctaattctttgggtatatacccagtaatgggattgctgggtcaaacagtatttctggttctagatccttgaggaatc
gccacactgtcttccacaatggttgaactaatttacacacccatcaacagtgtaaaatttttcctattcttccacatcctctccagcac
cttttgtttcctgactttttaataattgccattctaactggcatgagatggtatctcattgtggttttgatttgcatttctctaatgac
cagtgatgatgagcttcttttcatgtgtttcttggccacataaatgacttctttagagaagcatctgttcatatcctttgtccactttt
tgatggggtcgttaggttttttcttgtaaatttgttgaagttctttgtagattttggatgttagccctttgtcagatggatagattgca
aaaattttctcccattctgtaggttgcctgttcactctgatgatagtcttttgctgtgcagaagctctttagtttaattagatcccata
tgtcaattttggcctttgttgtcattgcttttgatgttttagtcgtgaatttttgcccatgcctatgtcctgaatggtattgcctaggt
tatcttctaggatttttatggttttaggttgcacatttaagtctttaatccaccttgagttaatttttgtataaggtgtaaggaagggg
tacagtttcagttttatgcatattgctagccagtttttccagcaccatttattaaatagggaattctttctccattgcttttgtgatgt
ttgtcaaagatcagatggtcgtagatgtgtggcattatttctgaggcttctgttctgttccactggtctatatatctgttttggtacca
gtaccatgctgtttttgttactgtagccttgtagtatagtttgaagtcaggtagcatcatgcctccagctttgttctttttgtttagga
ttgtcttggctatatgggctcttttttgattccatatgacatttaaagtagttttttctaattctttgaaaaaagtcagtggtagcttg
atggggatagcattgaatctataaattactttgggcagtatggccattttaaagatattgattctttctatctatgagcatggaatgtt
tttccatttgtttgtgtcctctcttatttccttgagcagtgagtggtttgtagctctccttgaagaggttcttcacatcccttagaagt
tgtatttctaggtattttattttattctctttgcagcaattgtgaatgggagttcacccatgatttggctctctgcttgtctattattg
gtgtataggaacgcttgtgatttctgcacactgattttgtatcttgagactttgctgaagctgtttatcagcttaagattttgggctga
gatgacagggtcttctaaatatacaatcatgtcatctgcaaacagagacaatttgacttcctctcttcctatttgaatatgctttattt
ctttctcttgcctgattgtcctggcgagaacttccaatactatgttgagtaagagtggcgagagggcatccttgtcttgtgccggtttt
caaagcaaatgatttttaaatttccatcttgatttcattgttgacccaatgatcattcaggagcaggttatttaatttccctgtatttg
catggttttgaaggttccttttgtagttgatttccaattttattctactgtggtctgagagagtgcttgatataatttcaatttttaaa
aatttattgaggcttgttttgtggcatatcatatggcctatcttggagaaagttccatgtgctgatgaatagaatgtgtattctgcagt
tgttgggtagaatgtcctgtaaatatctgttaagtccatttgttctttaaatccattgtttctttgtagactgtcttgatgacctgcct
agtgcagtcagtggagtattgaagtcccccactattattatgttgctgtctagtctagtagtaattgttttataaatttgggatctcca
gtattagatgcatatatattaagaattgtaatattctcccattggacaagggcttttatcattatatgatgtccctctttgtctttttt
aactgctgtttctttaaagtttgttttgtctgacataagaatagctgctttggctcgcttttggtgtccatttgtgtggaatgtcattt
tccacccctttaccttaagtttatgtgagtccttatgtgttaggtgagtctcctgaaggcggcagataactggttggtgaattcttatt
cattctgcaattctgtatcttttaagtggagcatttagtccatttacattcaacatcagtattgaggtgtgaggtactattccattctt
cgtggtatttgttgcctgtgtatctttttatctgtatttttgttgtatatgtcctatgggatttatgctttaaagaggttctgttttga
tgtgcttccagggtttatttcaagatttagagctccttttatcagttcttgtagtgttggcttggtagtgccgaattctctcagcattt
gtttttctgaaaaacactgtgtattttcttcatttgtgaagcttagtttcactggatataaaattcttggctgataattgttttgttta
agaaggctgaagatagggccatattcacttctagcttttacggtttctgctgagaaatctgctgttaatctgataggttttctttcata
ggttacctggtagtttcacctcacagctcttaagattctctttgtctttagataactttggatactctgatgacaatgtacctaggcaa
tgatatttttgcaatgaatttcccaggtgtttattgagcttcttgtatttggatatctaggtctctagcaaggtgggggaagttttcct
tgattatttccctggataagttttccaaacttttagatttctcttctttctcaggaatgctgattattcttaggtttgattgtttaaca
taatcccagatttcttggaggctttgttcatattttcttattcttttttctttgtctttgttggattgggttaattcaaaaactttgtc
ttcaagctctgaatttcttctgcttggattctattgctgagactttctagagcattttgcatttctataagtgcatccattcatccatt
gtttcctgaagttttgaatgttttttatttatgctatctctttaactgaagatttctcccctcatttcttgtatcatatttttggtttt
tttaaaattggacttcaccttcctcggatgcctccttgattagcttaataactgaccttctgaattatttttcaggtaaatcagggatt
tcttcttggtttggatgcattgctggtgagctagtatgattttttggggggtgttaaagaaccttgtttttcatattaccagagttagt
tttctggttccttctcacttgggtaggctctgtcagagggaaagtctaggcctcaaggctgagacttttgtcccatgaggtgttccctt
gatgtagcacagtcccccttttcctaggcgtggggcttcctgagagccgaactgtagtgattgttatctctcttctggatctagccacc
catcaggtctaccagactccaggctggtactggggtttgtctgcacagagtcttgtgacgtgaaccatctgtgggtctctcagccatag
atacaaccacctgctccaatggaggtggcagaggatgaaatggactctgtgagggtccttacttttggttgttcaatgcactatttttg
tgctggttggcctcctgccaggaggtggcactttctagaaagcatcagcagaggcagtcaggtggtggtggctgggggggctggggcac
cctagaactcccaagaatatatgccctttgtcttcagctaccagggtgagtaaggaaggaccatcaggtgggggcaggactagtcgtgt
ctgagctcagagtctccttgggcaggtctttctgtggctactgtgggaggatgggggtgtagtttccaggtcaatggatttatgttcct
aggacaattatggctgcctctgctgtgtcatgcaggtcatcaggaaagtgggggaaagcaagcagtcacgtgacttgcccagctcccat
gcaactcaaaaggttggtctcacttccagcgtgcaccctcccccgcaacagcaccgaatctgtttccatgcagtcagtgagcaaggctg
agaacttgccccaggctaccagctgcgaaaccaagtagggctgtcctacttccctgccagtggagtctgcacaccaaattcatgtcccc
ccaccaacccccccactgcccagcccctagatctggccaggtggagattttctttttcctgtcatcttttcccagttcctctggcagcc
ctcccaaatgacccctgtgaggcaaggcagaaatggcttcctaggggacccagagagcccacagggcttttcccgctgcttcctctacc
cctgtattttgcttggccctctaaattgactcagctccaggtaaggtcagaatcttctcctgtggtctagatcttcaggttccccagtg
aggatgtgtgtttgggggtagacggtcccccttttccacttccacagtttgggcactcacaatatttggggtgtttcccgggtcctgca
ggagcaatctgcttctttcagagggtgtgtgcgttctctcagctttcttgatttatttctgcaggtggttctgcaaaaaaaattcctga
tgggagacttcacatgctgctctgtgcatccgagtgggagctgcaatgtacttctgctgcctcccatctgccatcaccctctaatttgt
cggtaatatgcatttttaatcaatctttttttctctctctctctttttcttctcccccaaaactatactgccctttgatatcaaggaat
caaggacgtgatgttgaggggtgggcagtggatacactctttaccccttagggagctatatctagatttagatattgccaattcaagat
aacttaattgaaagcaaattcataatgaataCaCaCaCaCaCaCacacatctgcatgacaagatttttaatagttgaaagaataactaa
taattgtccacaggcaataagggctttttaagcaaaacagttgtgataaacaggtcattcttagaatagtaatccagccaatagtacag
gttgcttagagattatgtcattaccagagttaaaattctataatggcttctcactccctaccactgaggacaagtttatgtccttaggt
ttatgcttccctgaaacaataccacctgctattctccactttacatatcaacggcactggttctttatctaactctctggcacagcagg
agtttgttttcttctgcttcagagctttgaatttactatttcagcttctaaactttatttggcaatgccttcccatggcagattccttc
tgtcattttgcctctgttcgaatactttctccttaatttcattcttagttaataatatctgaaattattttgttgtttaacttaattat
taattttatgtatgttctacctagattataatcttcagaggaaagttttattctctgacttatttaacttaaatgcccactactttaaa
aattatgacatttatttaacagatatttgctgaacaaatgtttgaaaatacatgggaaagaatgcttgaaaacacttgaaattgcttgt
gtaaagaaacagttttatcagttaggatttaatcaatgtcagaagcaatgatataggaaaaatcgaggaataagacagttatggataag
gagaaatcaacaaactcttaaaagatattgcctcaaaagcataagaggaaataagggtttatacatgacttttagaacactgccttggt
ttttggataaatggggaagttgtttgaaaacaggagggatcctagatattccttagtctgaggaggagcaattaagattcacttgttta
gaggctgggagtggtggctcacgcctgtaatcccagaattttgggaggccaaggcaggcagatcacctgaggtcaagagttcaagacca
acctggccaacatggtgaaatcccatctctacaaaaatacaaaaattagacaggcatgatggcaagtgcctgtaatcccagctacttgg
gaggctgaggaaggagaattgcttgaacctggaaggcaggagttgcagtgagccgagatcataccactgcactccagcctgggtgacag
aacaagactctgtctcaaaaaaaaaaaagagagattcaaaagattcacttgtttaggccttagcgggcttagacaccagtctctgacac
attcttaaaggtcaggctctacaaatggaacccaaccagactctcagatatggccaaagatctatacacacccatctcacagatcccct
atcttaaagagaccctaatttgggttcacctcagtctctataatctgtaccagcataccaataaaaatctttctcacccatccttagat
tgagagaagtcacttattattatgtgagtaactggaagatactgataagttgacaaatctttttctttcctttcttattcaacttttat
tttaacttccaaagaacaagtgcaatatgtgcagctttgttgcgcaggtcaacatgtatctttctggtcttttagccgcctaacacttt
gagcagatataagccttacacaggattatgaagtctgaaaggattccaccaatattattataattcctatcaacctgataggttagggg
aaggtagagctctcctccaataagccagatttccagagtttctgacgtcataatctaccaaggtcatggatcgagttcagagaaaaaac
aaaagcaaaaccaaacctaccaaaaaataaaaatcccaaagaaaaaataaagaaaaaaacagcatgaatacttcctgccatgttaagtg
gccaatatgtcagaaacagcactgagttacagataaagatgtctaaactacagtgacatcccagctgtcacagtgtgtggactattagt
caataaaacagtccctgcctcttaagagttgttttccatgcaaatacatgtcttatgtcttagaataagattccctaagaagtgaacct
agcatttatacaagataattaattctaatccatagtatctggtaaagagcattctaccatcatctttaccgagcatagaagagctacac
caaaaccctgggtcatcagccagcacatacacttatccagtgataaatacacatcatcgggtgcctacatacatacctgaatataaaaa
aaatacttttgctgagatgaaacaggcgtgatttatttcaaataggtacggataagtagatattgaagtaaggattcagtcttatatta
tattacataacattaatctattcctgcactgaaactgttgctttataggatttttcactacactaatgagaacttaagagataatggcc
taaaaccacagagagtatattcaaagataagtatagcacttcttatttggaaaccaatgcttactaaatgagactaagacgtgtcccat
caaaaatcctggacctatgcctaaaacacatttcacaatccctgaacttttcaaaaattggtacatgctttaactttaaactacaggcc
tcactggagctacagacaagaaggtgaaaaacggctgacaaaagaagtcctggtatcttctatggtgggagaagaaaactagctaaagg
gaagaataaattagagaaaaattggaatgactgaatcggaacaaggcaaaggctataaaaaaaattaagcagcagtatcctcttggggg
ccccttccccacactatctcaatgcaaatatctgtctgaaacggtccctggctaaactccacccatgggttggccagccttgccttgac
caatagccttgacaaggcaaacttgaccaatagtcttagagtatccagtgaggccaggggccggcggctggctagggatgaagaataaa
aggaagcacccttcagcagttccacacactcgcttctggaacgtctgaggttatcaataagctcctagtccagacgccatgggtcattt
cacagaggaggacaaggctactatcacaagcctgtggggcaaggtgaatgtggaagatgctggaggagaaaccctgggaaggtaggctc
tggtgaccaggacaagggagggaaggaaggaccctgtgcctggcaaaagtccaggtcgcttctcaggatttgtggcaccttctgactgt
caaactgttcttgtcaatctcacaggctcctggttgtctacccatggacccagaggttctttgacagctttggcaacctgtcctctgcc
tctgccatcatgggcaaccccaaagtcaaggcacatggcaagaaggtgctgacttccttgggagatgccataaagcacctggatgatct
caagggcacctttgcccagctgagtgaactgcactgtgacaagctgcatgtggatcctgagaacttcaaggtgagtccaggagatgttt
cagcactgttgcctttagtctcgaggcaacttagacaactgagtattgatctgagcacagcagggtgtgagctgtttgaagatactggg
gttgggagtgaagaaactgcagaggactaactgggctgagacccagtggcaatgttttagggcctaaggagtgcctctgaaaatctaga
tggacaactttgactttgagaaaagagaggtggaaatgaggaaaatgacttttctttattagatttcggtagaaagaactttcaccttt
cccctatttttgttattcgttttaaaacatctatctggaggcaggacaagtatggtcattaaaaagatgcaggcagaaggcatatattg
gctcagtcaaagtggggaactttggtggccaaacatacattgctaaggctattcctatatcagctggacacatataaaatgctgctaat
gcttcattacaaacttatatcctttaattccagatgggggcaaagtatgtccaggggtgaggaacaattgaaacatttgggctggagta
gattttgaaagtcagctctgtgtgtgtgtgtgtgtgtgtgcgcgcgtgtgtttgtgtgtgtgtgagagcgtgtgtttcttttaacgttt
tcagcctacagcatacagggttcatggtggcaagaagataacaagatttaaattatggccagtgactagtgctgcaagaagaacaacta
cctgcatttaatgggaaagcaaaatctcaggctttgagggaagttaacataggcttgattctgggtggaagcttggtgtgtagttatct
ggaggccaggctggagctctcagctcactatgggttcatctttattgtctcctttcatctcaacagctcctgggaaatgtgctggtgac
cgttttggcaatccatttcggcaaagaattcacccctgaggtgcaggcttcctggcagaagatggtgactggagtggccagtgccctgt
cctccagataccactgagctcactgcccatgatgcagagctttcaaggataggctttattctgcaagcaatcaaataataaatctattc
tgctaagagatcacacatggttgtcttcagttctttttttatgtctttttaaatatatgagccacaaagggttttatgttgagggatgt
gtttatgtgtatttatacatggctatgtgtgtttgtgtcatgtgcacactccacacttttttgtttacgttagatgtgggttttgatga
gcaaataaaagaactaggcaataaagaaacttgtacatgggagttctgcaagtgggagtaaaaggtgcaggagaaatctggttggaaga
aagacctctataggacaggactcctcagaaacagatgttttggaagagatggggaaaggttcagtgaagggggctgaacccccttccct
ggattgcagcacagcagcgaggaaggggctcaacgaagaaaaagtgttccaagctttaggaagtcaaggtttaggcagggatagccatt
ctattttattaggggcaatactatttccaacggcatctggcttttctcagcccttgtgaggctctacagggaggttgaggtgttagaga
tcagagcaggaaacaggtttttctttccacggtaactacaatgaagtgatccttactttactaaggaacttttcattttaagtgttgac
gcatgcctaaagaggtgaaattaatcccatacccttaagtctacagactggtcacagcatttcaaggaggagacctcattgtaagcttc
tagggaggtggggacttaggtgaaggaaatgagccagcagaagctcacaagtcagcatcagcgtgtcatgtctcagcagcagaacagca
cggtcagatgaaaatatagtgtgaagaatttgtataacattaattgagaaggcagattcactggagttcttatataattgaaagttaat
gcacgttaataagcaagagtttagtttaatgtgatggtgttatgaacttaacgcttgtgtctccagaaaattcacatgctgaatcccca
actcccaattggctccatttgtgggggaggctttggaaaagtaatcaggtttagaggagctcatgagagcagatccccatcatagaatt
attttcctcatcagaagcagagagattagccatttctcttccttctggtgaggacacagtgggaagtcagccacctgcaacccaggaag
agagccctgaccaggaaccagcagaaaagtgagaaaaaatcctgttgttgaagtcacccagtctatgctattttgttatagcaccttgc
actaagtaaggcagatgaagaaagagaaaaaaataagcttcggtgttcagtggattagaaaccatgtttatctcaggtttacaaatctc
cacttgtcctctgtgtttcagaataaaataccaactctactactctcatctgtaagatgcaaatagtaagcctgagcccttctgtctaa
ctttgaattctattttttcttcaacgtactttaggcttgtaatgtgtttatatacagtgaaatgtcaagttctttctttatatttcttt
ctttcttttttttcctcagcctcagagttttccacatgcccttcctactttcaggaacttctttctccaaacgtcttctgcctggctcc
atcaaatcataaaggacccacttcaaatgccatcactcactaccatttcacaattcgcactttctttctttgtcctttttttttttagt
aaaacaagtttataaaaaattgaaggaataaatgaatggctacttcataggcagagtagacgcaagggctactggttgccgatttttat
tgttatttttcaatagtatgctaaacaaggggtagattatttatgctgcccatttttagaccataaaagataacttcctgatgttgcca
tggcatttttttccttttaattttatttcatttcattttaatttcgaaggtacatgtgcaggatgtgcaggcttgttacatgggtaaat
gtgtgtctttctggccttttagccatctgtatcaatgagcagatataagctttacacaggatcatgaaggatgaaagaatttcaccaat
attataataatttcaatcaacctgatagcttaggggataaactaatttgaagatacagcttgcctccgataagccagaattccagagct
tctggcattataatctagcaaggttagagatcatggatcactttcagagaaaaacaaaaacaaactaaccaaaagcaaaacagaaccaa
aaaaccaccataaatacttcctaccctgttaatggtccaatatgtcagaaacagcactgtgttagaaataaagctgtctaaagtacact
aatattcgagttataatagtgtgtggactattagtcaataaaaacaacccttgcctctttagagttgttttccatgtacacgcacatct
tatgtcttagagtaagattccctgagaagtgaacctagcatttatacaagataattaattctaatccacagtacctgccaaagaacatt
ctaccatcatctttactgagcatagaagagctacgccaaaaccctgggtcatcagccagcacacacacttatccagtggtaaatacaca
tcatctggtgtatacatacatacctgaatatggaatcaaatatttttctaagatgaaacagtcatgatttatttcaaataggtacggat
aagtagatattgaggtaagcattaggtcttatattatgtaacactaatctattactgcgctgaaactgtggctttatagaaattgtttt
cactgcactattgagaaattaagagataatggcaaaagtcacaaagagtatattcaaaaagaagtatagcactttttccttagaaacca
ctgctaactgaaagagactaagatttgtcccgtcaaaaatcctggacctatgcctaaaacacatttcacaatccctgaacttttcaaaa
attggtacatgctttagctttaaactacaggcctcactggagctagagacaagaaggtaaaaaacggctgacaaaagaagtcctggtat
cctctatgatgggagaaggaaactagctaaagggaagaataaattagagaaaaactggaatgactgaatcggaacaaggcaaaggctat
aaaaaaaattagcagtatcctcttgggggccccttccccacactatctcaatgcaaatatctgtctgaaacggtccctggctaaactcc
acccatgggttggccagccttgccttgaccaatagccttgacaaggcaaacttgaccaatagtcttagagtatccagtgaggccagggg
ccggcggctggctagggatgaagaataaaaggaagcacccttcagcagttccacacactcgcttctggaacgtctgaggttatcaataa
gctcctagtccagacgccatgggtcatttcacagaggaggacaaggctactatcacaagcctgtggggcaaggtgaatgtggaagatgc
tggaggagaaaccctgggaaggtaggctctggtgaccaggacaagggagggaaggaaggaccctgtgcctggcaaaagtccaggtcgct
tctcaggatttgtggcaccttctgactgtcaaactgttcttgtcaatctcacaggctcctggttgtctacccatggacccagaggttct
ttgacagctttggcaacctgtcctctgcctctgccatcatgggcaaccccaaagtcaaggcacatggcaagaaggtgctgacttccttg
ggagatgccacaaagcacctggatgatctcaagggcacctttgcccagctgagtgaactgcactgtgacaagctgcatgtggatcctga
gaacttcaaggtgagtccaggagatgtttcagccctgttgcctttagtctcgaggcaacttagacaacggagtattgatctgagcacag
cagggtgtgagctgtttgaagatactggggttgggggtgaagaaactgcagaggactaactgggctgagacccagtggtaatgttttag
ggcctaaggagtgcctctaaaaatctagatggacaattttgactttgagaaaagagaggtggaaatgaggaaaatgacttttctttatt
agattccagtagaaagaactttcatctttccctcatttttgttgttttaaaacatctatctggaggcaggacaagtatggtcgttaaaa
agatgcaggcagaaggcatatattggctcagtcaaagtggggaactttggtggccaaacatacattgctaaggctattcctatatcagc
tggacacatataaaatgctgctaatgcttcattacaaacttatatcctttaattccagatgggggcaaagtatgtccaggggtgaggaa
caattgaaacatttgggctggagtagattttgaaagtcagctctgtgtgtgtgtgtgtgtgtgcgcgcgcgcgtgtgtgtgtgtgtgtc
agcgtgtgtttcttttaacgtcttcagcctacaacatacagggttcatggtggcaagaagatagcaagatttaaattatggccagtgac
tagtgcttgaaggggaacaactacctgcatttaatgggaaggcaaaatctcaggctttgagggaagttaacataggcttgattctgggt
ggaagcttggtgtgtagttatctggaggccaggctggagctctcagctcactatgggttcatctttattgtctcctttcatctcaacag
ctcctgggaaatgtgctggtgaccgttttggcaatccatttcggcaaagaattcacccctgaggtgcaggcttcctggcagaagatggt
gactgcagtggccagtgccctgtcctccagataccactgagctcactgcccatgattcagagctttcaaggataggctttattctgcaa
gcaatacaaataataaatctattctgctgagagatcacacatgattttcttcagctcttttttttacatctttttaaatatatgagcca
caaagggtttatattgagggaagtgtgtatgtgtatttctgcatgcctgtttgtgtttgtggtgtgtgcatgctcctcatttattttta
tatgagatgtgcattttgatgagcaaataaaagcagtaaagacacttgtacacgggagttctgcaagtgggagtaaatggtgtaggaga
aatccggtgggaagaaagacctctataggacaggacttctcagaaacagatgttttggaagagatgggaaaaggttcagtgaagacctg
ggggctggattgattgcagctgagtagcaaggatggttcttaaggaagggaaagtgttccaagctttaggaattcaaggtttagtcagg
tgtagcaattctattttattaggaggaatactatttctaatggcacttagcttttcacagcccttgtggatgcctaagaaagtgaaatt
aatcccatgccctcaagtgtgcagattggtcacagcatttcaagggagagacctcattgtaagactctgggggaggtggggacttaggt
gtaagaaatgaatcagcagaggctcacaagtcagcatgagcatgttatgtctgagaaacagaccagcactgtgagatcaaaatgtagtg
ggaagaatttgtacaacattaattggaaggcttacttaatggaatttttgtatagttggatgttagtgcatctctataagtaagagttt
aatatgatggtgttacggacctaatgtttgtgtctcctcaaaattcacatgctgaatccccaactcccaactgaccttatctgtggggg
aggcttttgaaaagtaattaggtttagatgagctcataagagcagatccccatcataaaattattttccttatcagaagcagagagaca
agccatttctctttcctcccggtgaggacacagtgagaagtccgccatctgcaatccaggaagagaaccctgaccacgagtcagccttc
agaaatgtgagaaaaaactctgttgttgaagccacccagtcttttgtattttgttatagcaccttgcactgagtaaggcagatgaagaa
ggagaaaaaaataagcttgggttttgagtggactacagaccatgtttatctcaggtttgcaaagctcccctcgtcccctatgtttcagt
ataaaatacctactctactactctcatctataagacccaaataataagcctgcgcccttctctctaactttgatttctcctatttttac
ttcaacatgctttactctagccttgtaatgtctttacatacagtgaaatgtaaagttctttattctttttttctttctttcttttttct
cctcagcctcagaatttggcacatgcccttccttctttcaggaacttctccaacatctctgcctggctccatcatatcataaaggtccc
acttcaaatgcagtcactaccgtttcagaatatgcactttctttcttttttgttttttgttttttttaagtcaaagcaaatttcttgag
agagtaaagaaataaacgaatgactactgcataggcagagcagccccgagggccgctggttgttccttttatggttatttcttgatgat
atgttaaacaagttttggattatttatgccttctctttttaggccatatagggtaactttctgacattgccatggcatttttcttttaa
tttaatttactgttaccttaaattcaggggtacacgtacaggatatgcaggtttgttttataggtaaaagtgtgccatggttttaatgg
gttttttttttcttgtaaagttgtttaagtttcttgtttactctggatattaggcctttgtcagaagaatagattggaaaatctttttc
ccattctgtagattgtctttcgctctgatggtagtttcttttgctgagcaggagctctttagtttaattagattccattggtcaatttt
tgcttttgctgcaattgcttttcacgctttcatcatgaaatctgtgcccgtgtttatatcatgaatagtattgccttgatttttttcta
ggctttttatagtttggggtttttcatttaagtctctaatccatctggagttaattttggataaggtataaggaaggagtccagtttca
tttttcagcatatggctagccagttctcccccatcatttattaaattgaaaatcctttccccattgcttgcttttgtcaggtttctaaa
agaccagatggttgtaggtacaatatgcagtttcttcaagtcatataataccatctgaaatctcttattaattcatttcttttagtatg
tatgctggtctcctctgctcactatagtgagggcaccattagccagagaatctgtctgtctagttcatgtaagattctcagaattaaga
aaaatggatggcatatgaatgaaacttcatggatgacatatggaatctaatatgtatttgttgaattaatgcataagatgcaacagaga
gaagttgacaactgcaatgataacctggtattgatgatataagagtctatagatcacagtagaagcaataatcatggaaaacaattgga
aatggggaacagccacaaacaagaaagaatcaatacttccaggaaagtgactgcaggtcacttttcctggagcgggtgagagaaaagtg
gaagttagcagtaactgctgaattcctggttggctgatggaaagatggggcagctgttcactggtacgcagggttttagatgtatgtac
ctaaggatatgaggtatggcaatgaacagaaattcttttgggaatgagttttagggccattaaaggacatgacctgaagtttcctctga
ggccagtccccacaactcaatataaatgtgtttcctgcatatagtcaaagttgccacttctttttcttcatatcatcgatctctgctct
taaagataatcttggttttgcctcaaactgtttgtcactacaaactttccccatgttcctaagtaaaacaggtaactgcctctcaacta
tatcaagtagactaaaatattgtgtctctaatatcagaaattcagctttaatatattgggtttaactctttgaaatttagagtctcctt
gaaatacacatgggggtgatttcctaaactttatttcttgtaaggatttatctcaggggtaacacacaaaccagcatcctgaacctcta
agtatgaggacagtaagccttaagaatataaaataaactgttcttctctctgccggtggaagtgtgccctgtctattcctgaaattgct
tgtttgagacgcatgagacgtgcagcacatgagacacgtgcagcagcctgtggaatattgtcagtgaagaatgtctttgcctgattaga
tataaagacaagttaaacacagcattagactatagatcaagcctgtgccagacacaaatgacctaatgcccagcacgggccacggaatc
tcctatcctcttgcttgaacagagcagcacacttctcccccaacactattagatgttctggcataattttgtagatatgtaggatttga
catggactattgttcaatgattcagaggaaatctcctttgttcagataagtacactgactactaaatggattaaaaaacacagtaataa
aacccagttttccccttacttccctagtttgtttcttattctgctttcttccaagttgatgctggatagaggtgtttatttctattcta
aaaagtgatgaaattggccgggcgcggtggctcacacctgtaatcccagcactttgggaggctgaggtgggcggatcacgaggtcagga
gatcaagaccatcctggctaacatggtgaaaccccatctctactaaaaatacaaaaaattagccagagacagtggcgggtgcctgtagt
cccagctactcgggaggctgaggcaggagaatggcgtgaacctgggaggcagagcttgcggtgagcagagatcgcgccactgcacactc
cagcctgggtgacaaagcgagactccatctcaaaaaaaaaaaaaaaaaaaagaaaaagaaagaaagaaagaaaaaaaaactgatgaaat
tgtgtattcaatgtagtctcaagagaattgaaaaccaagaaaggctgtggcttcttccacataaagcctggatgaataacaggataaca
cgttgttacattgtcacaactcctgatccaggaattgatggctaagatattcgtaattcttatccttttcagttgtaacttattcctat
ttgtcagcattcaggttattagcggctgctggcgaagtccttgagaaataaactgcacactggatggtgggggtagtgtaggaaaatgg
aggggaaggaagtaaagtttcaaattaagcctgaacagcaaagttcccctgagaaggccacctggattctatcagaaactcgaatgtcc
atcttgcaaaacttccttgcccaaaccccacccctggagtcacaacccacccttgaccaatagattcattttactgagggaggcaaagg
gctggtcaatagattcatttcactgggagaggcaaagggctgggggccagagaggagaagtaaaaagccacacatgaagcagcaatgca
ggcatgcttctggctcatctgtgatcaccaggaaactcccagatctgacactgtagtgcatttcactgctgacaagaaggctgctgcca
ccagcctgtgaagcaaggttaaggtgagaaggctggaggtgagattctgggcaggtaggtactggaagccgggacaaggtgcagaaagg
cagaaagtgtttctgaaagagggattagcccgttgtcttacatagtctgactttgcacctgctctgtgattatgactatcccacagtct
cctggttgtctacccatggacctagaggtactttgaaagttttggatatctgggctctgactgtgcaataatgggcaaccccaaagtca
aggcacatggcaagaaggtgctgatctccttcggaaaagctgttatgctcacggatgacctcaaaggcacctttgctacactgagtgac
ctgcactgtaacaagctgcacgtggaccctgagaacttcctggtgagtagtaagtacactcacgctttcttctttacccttagatattt
gcactatgggtacttttgaaagcagaggtggctttctcttgtgttatgagtcagctatgggatatgatatttcagcagtgggattttga
gagttatgttgctgtaaataacataactaaaatttggtagagcaaggactatgaataatggaaggccacttaccatttgatagctctga
aaaacacatcttataaaaaattctggccaaaatcaaactgagtgtttttggatgagggaacagaagttgagatagagaaaataacatct
ttcctttggtcagcgaaattttctataaaaattaatagtcacttttctgcatagtcctggaggttagaaaaagatcaactgaacaaagt
agtgggaagctgttaaaaagaggattgtttccctccgaatgatgatggtatacttttgtacgcatggtacaggattctttgttatgagt
gtttgggaaaattgtatgtatgtatgtatgtatgtatgtgatgactggggacttatcctatccattactgttccttgaagtactattat
cctactttttaaaaggacgaagtctctaaaaaaaaaatgaaacaatcacaatatgttggggtagtgagttggcatagcaagtaagagaa
ggataggacacaatgggaggtgcagggctgccagtcatattgaagctgatatctagcccataatggtgagagttgctcaaactctggtg
aaaaaggatgtaagtgttatatctatttactgcaagtccagcttgaggccttctattcactatgtaccattttcttttttatcttcact
ccctccccagctcttaggcaacgtgatattgattgttttggcaacccacttcagcgaggattttaccctacagatacaggcttcttggc
agtaactaacaaatgctgtggttaatgctgtagcccacaagaccactgagttccctgtccactatgtttgtacctatggtccactatgt
ttgtacctatgtcccaaaatctcatctcctttagatgggggaggttggggagaagagcagtatcctgcctgctgattcagttcctgcat
gataaaaatagaataaagaaatatgctctctaagaaatatcattgtactctttttctgtctttatattttaccctgattcagccaaaag
gacgcactatttctgatggaaatgagaatgttggagaatgggagtttaaggacagagaagatactttcttgcaatcctgcaagaaaaga
gagaactcgtgggtggatttagtggggtagttactcctaggaaggggaaatcgtctctagaataagacaatgtttttacagaaagggag
gtcaatggaggtactctttggaggtgtaagaggattgttggtagtgtgtagaggtatgttaggactcaaattagaagttctgtataggc
tattatttgtatgaaactcaggatatagctcatttggtgactgcagttcacttctacttattttaaacaacatattttttattatttat
aatgaagtggggatggggcttcctagagaccaatcaagggccaaaccttgaactttctcttaacgtcttcaatggtattaatagagaat
tatctctaaggcatgtgaactggctgtcttggttttcatctgtacttcatctgctacctctgtgacctgaaacatatttataattccat
taagctgtgcatatgatagatttatcatatgtattttccttaaaggatttttgtaagaactaattgaattgatacctgtaaagtcttta
tcacactacccaataaataataaatctctttgttcagctctctgtttctataaatatgtacaagttttattgtttttagtggtagtgat
tttattctctttctatatatatacacacacatgtgtgcattcataaatatatacaatttttatgaataaaaaattattagcaatcaata
ttgaaaaccactgatttttgtttatgtgagcaaacagcagattaaaaggctgagatttaggaaacagcacgttaagtcaagttgataga
ggagaatatggacatttaaaagaggcaggatgatataaaattagggaaactggatgcagagaccagatgaagtaagaaaaatagctatc
gttttgagcaaaaatcactgaagtttcttgcatatgagagtgacataataaatagggaaacgtagaaaattgattcacatgtatatata
tatatagaactgattagacaaagtctaacttgggtatagtcagaggagcttgctgtaattatattgaggtgatggataaagaactgaag
ttgatggaaacaatgaagttaagaaaaaaaatcgagtaagagaccattgtggcagtgattgcacagaactggaaaacattgtgaaacag
agagtcagagatgacagctaaaatccctgtctgtgaatgaaaagaaggaaatttattgacagaacagcaaatgcctacaagccccctgt
ttggatctggcaatgaacgtagccattctgtggcaatcacttcaaactcctgtacccaagacccttaggaagtatgtagcaccctcaaa
cctaaaacctcaaagaaagaggttttagaagatataataccctttcttctccagtttcattaatcccaaaacctctttctcaaagtatt
tcctctatgtgtccaccccaaagagctcacctcaccatatctcttgagtgggagcacatagataggcggtgctaccatctaacagcttc
tgaaattcctttgtcatatttttgagtccccactaataacccacaaagcagaataaataccagttgctcatgtacaataatcactcaac
tgctgtcttgtagcatacattaattaagcacattctttgaataattactgtgtccaaacaatcacactttaaaatctcacacttgtgct
atcccttgcccttctgaatgtcactctgtattttaaatgaagagatgagggttgaatttcctgtgttacttattgttcatttctcgatg
aggagttttcacattcacctttagtggaaaacacataagtacacatcttacaggaaaaatataccaaactgacatgtagcatgaatgct
tgtgcatgtagtcatataaaatcttgtagcaatgtaaacattctctgatatacacatacagatgtgtctatatgtctacacaatttctt
atgctccatgaacaaacattccatgcacacataagaacacacactgttacagatgcatacttgagtgcattgacaaaattaccccagtc
aatctagagaatttggatttctgcatttgactctgttagctttgtacatgctgttcatttactctgggtgatgtctttccctcattttg
ccttgtctatcttgtactcatactttaagtcctaacttatatgttatctcaactaagaagctatttttttttaattttaactgggctta
aagccctgtctataaactctgctacaattatgggctctttcttataatatttagtgtttttcctactaatgtacttaatctgctcattg
tatattcctaccactaaattttaacctcttttatggtagagacattgtcttgtaaactcttatttccctagtatttggagatgaaaaaa
aagattaaattatccaaaattagatctctcttttctacattatgagtattacactatccatagagaagtttgtttgagacctaaactga
ggaacctttggttctaaaatgactatgtgatatcttagtatttataggtcatgaggttccttcctctgcctctgctatagtttgattag
tcaacaagcatgtgtcatgcatttattcacatcagaatttcatacactaataagacatagtatcagaagtcagtttattagttatatca
gttagggtccatcaaggaaaggacaaaccattatcagttactcaacctagaattaaatacagctcttaatagttaattatccttgtatt
ggaagagctaaaatatcaaataaaggacagtgcagaaatctagatgttagtaacatcagaaaacctcttccgccattaggcctagaagg
gcagaaggagaaaatgtttataccaccagagtccagaaccagagcccataaccagaggtccactggattcagtgagctagtgggtgctc
cttggagagagccagaactgtctaatgggggcatcaaagtatcagccataaaaaaccataaaaaagactgtctgctgtaggagatccgt
tcagagagagagagagaccagaaataatcttgcttatgctttccctcagccagtgtttaccattgcagaatgtacatgcgactgaaagg
gtgaggaaacctgggaaatgtcagttcctcaaatacagagaacactgagggaaggatgagaaataaatgtgaaagcagacatgaatggt
aattgacagaaggaaactaggatgtgtccagtaaatgaataattacagtgtgcagtgattattgcaatgattaatgtattgataagata
atatgaaaacacagaattcaaacagcagtgaactgagattagaattgtggagagcactggcatttaagaatgtcacacttagaatgtgt
ctctaggcattgttctgtgcatatatcatctcaatattcattatctgaaaattatgaattaggtacaaagctcaaataatttatttttt
caggttagcaagaactttttttttttttttctgagatagagcattgctatggttgcccaggctggagtgcaatggcatgatccaggctc
actgcaacatctgcctcccaggttcaagcgattctcctgcctcagcctcccaagtagctggcactacaggcatgtgccaccaccatgcc
tggctaattttctatttttagtagatagggggtttcaccatgttggtcaggctgatctcgaactcctaacatcaggtgatccaccctcc
tcggcctctgaaagtgctgggatcacaggcgtgagccaccacacccagccaagaatgtgaattttgtagaaggatataacccatatttc
tctgaccctagagtccttagtatacctcccataccatgtggctcatcctccttacatacatttcccatctttcaccctaccttttcctt
tttgtttcagcttttcactgtgtcaaaatctagaaccttatctcctacctgctctgaaaccaacagcaagttgacttccattctaaccc
acattggcattacactaattaaaatcgatactgagttctaaaatcatcggggattttggggactatgtcttacttcatacttccttgag
atttcacattaaatgttggtgttcattaaaggtccttcatttaactttgtattcatcacactcttggattcacagttatatctaaactc
ttaaatacagcctgtataatcccaattcccaactctgatttctaacctctgacctccaacctcagtgccaaacccatatatcaaacaat
gtactgggcttatttatatagatgtcctataggcacctcagactcagcatgggtatttcacttgttatactaaaactgtttctcttcca
gtgttttccattttagtcattagatagctacttgcccattcaccaaggtcacagattaaaatcatttccctacctctaatcaacagttc
gattctgcttcaatttgtccctatctattaatcaccactcttactgcccagtcaggtcctcattgtttcctgaacaagagtagatgcta
ttctttccacttttagaccttatcctggctggatgcggtggctcaggcttgtaaacccagcactttgggaggccaaggcaggcagatca
cttgaggtcaggagttcaagaccagcctgaccaacatggtgaaaccccatctctactaaaaatacaaaatcagccgggcgtgtggtgca
tgcctgcagtcccagctattcaggtggctgaggcaggagaattgcttgaacccaggaggcagaggttgcggtgagcctagattgcacca
ttgcactctagcttgggcaatagggatgaaactccatctcagaagagaaaagaaaaaaagaccttattctgttatacaaatcctctcaa
tgcaatccatatagaataaacatgtaaccagatctcccaatgtgtaaaatcatttcaggtagaacagaattaaagtgaaaagccaagtc
tttggaattaacagacaaagatcaaataacagtcctcatggccttaagaatttacctaacattttttttagaatcaattttcttatata
tgaattggaaacataattcctccctcacaaacacattctaagattttaaggagatattgatgaagtacatcatctgtcatttttaacag
gtagtggtagtgattcacacagcacattatgatctgttcttgtatgttctgttccattctgtattcttgacctggttgtattctttctg
agctccagatccacatatctaagtacatctttttgcattttacaagagtgcatacaatacaatgtatccaagactgtatttctgatttt
atcgtaccactaaactcacaaatgtggccctattcttgtgttcacgactgacatcaccgtcatggtccaagtctgataatagaaatggc
attgtcactttcttccctactgcaacagaagcccagctatttgtctcccattttctctacttctaaaatacatttcttcactaagtgag
aataatcttttaaagacacaaatcaaaccatgccaccacctttcttgaattattcaatatctttcgttggcttccaggttacagaaaaa
taacttgtaacaaagtttaaaggtcattcatggctcctctctaccctattttataacatttccccttgtgatcagaatctcaggcacat
catccatctttctatatacaaataaagtcatatagtttgaactcacctctggttacttttaatcaaccaaatgctgtaaaatgcatttg
tatcgctacgtgttaagcagtagttgattcttttcatttctgtgtaatattctattctttgactataccgtaatttatcaattctactg
ttggtaagcatttaagtggctaccggtttgaggtttttatgattattgctgtcataagcatttctatacatgtctttggatacacacat
gcatgtgtttctgaatatctaaaaatgtaattgctaggtaatagacttatcaagcatccagcatttgtggatactattaaaggttttcc
aaaggggttatactattgtacagtgtcaccaacagagtttgagtttctattgatccatatcaccaccaaaatttgaactgtcagtctta
tctcttctcttgtctcttttttcctcttttttttccttcccttcccctctcttcgtttcttttctctcctcttctcttctttcctctct
tcccttccctttctctttctcttccctatcccttctcctctcctctcccctccttttttctcctctcctctccattatttatttttcct
tcttctcctccatcccttccatcctctctcttcccctcttccttccttcctttctccatttcttcctcctctttccttcaatccttcct
tttggatatgctcatgggtgtgtatttgtctgccattgtggcattatttgaattcagaaaagagtgaaaaactactgggatcttcattc
ctgggtctaattccacatttttttttaagaacacatctgtaaaaatgttctgtactagcatattcccaggaacttcgttaaatttaatc
tggctgaatatggtaaatctacttttcactttgcattctttctttagtcataccataattttaaacattcaaaatatttgtatataata
tttgattttatctgtcattaaaatgttaaccttaaaattcatgtttccagaacctatttcaataactggtaaataaacactattcattt
tttaaatattcttttaatggatatttatttcaatataataaaaaattagagttttattataggaagaatttaccaaaagaaggaggaag
caagcaagtttaaactgcagcaatagatttgtccattccaacctctcaaaattcccttggagacaaaaatctctagaggcaaagaagaa
ctttatattgagtcaacttgttaaaacatctgcttttagataagttttcttagtataaagtgacagaaacaaataagttaaactctaag
atacattccactatattagcctaaaacacttctgcaaaaatgaaactaggaggatatttttagaaacaactgctgaaagagatgcggtg
gggagatatgtagaggagaacagggtttctgagtcaagacacacatgacagaacagccaatctcagggcaagttaagggaatagtggaa
tgaaggttcatttttcattctcacaaactaatgaaaccctgcttatcttaaaccaacctgctcactggagcagggaggacaggaccagc
ataaaaggcagggcagagtcgactgttgcttacactttcttctgacataacagtgttcactagcaacctcaaacagacaccatggtgca
tctgactcctgaggagaagactgctgtcaatgccctgtggggcaaagtgaacgtggatgcagttggtggtgaggccctgggcaggttgg
tatcaaggttataagagaggctcaaggaggcaaatggaaactgggcatgtgtagacagagaagactcttgggtttctgataggcactga
ctctctgtcccttgggctgttttcctaccctcagattactggtggtctacccttggacccagaggttctttgagtcctttggggatctg
tcctctcctgatgctgttatgggcaaccctaaggtgaaggctcatggcaagaaggtgctaggtgcctttagtgatggcctggctcacct
ggacaacctcaagggcactttttctcagctgagtgagctgcactgtgacaagctgcacgtggatcctgagaacttcagggtgagtccag
gagatgcttcacttttctctttttactttctaatcttacattttggttcttttacctacctgctcttctcccacatttttgtcatttta
ctatattttatcatttaatgcttctaaaattttgttaattttttatttaaatattctgcattttttccttcctcacaatcttgctattt
taaattatttaatatcctgtctttctctcccaaccccctcccttcatttttccttctctaacaacaactcaaattatgcataccagctc
tcacctgctaattctgcacttagaataatccttttgtctctccacatgggtatgggagaggctccaactcaaagatgagaggcatagaa
tactgttttagaggctataaatcattttacaataaggaataattggaattttataaattctgtagtaaatggaatggaaaggaaagtga
atatttgattatgaaagactaggcagttacactggaggtggggcagaagtcgttgctaggagacagcccatcatcacactgattaatca
attaatttgtatctattaatctgtttatagtaattaatttgtatatgctatatacacatacaaaattaaaactaatttggaattaattt
gtatatagtattatacagcatatatagcatatatgtacatatatagactacatgctagttaagtacatagaggatgtgtgtgtatagat
atatgttatatgtatgcattcatatatgtacttatttatgctgatgggaataacctggggatcagttttgtctaagatttgggcagaaa
aaaatgggtgttggctcagtttctcagaagccagtctttatttctctgttaaccatatgcatgtatctgcctacctcttctccgcagct
cttgggcaatgtgctggtgtgtgtgctggcccgcaactttggcaaggaattcaccccacaaatgcaggctgcctatcagaaggtggtgg
ctggtgtggctaatgccctggctcacaagtaccattgagatcctggactgtttcctgataaccataagaagaccctatttccctagatt
ctattttctgaacttgggaacacaatgcctacttcaagggtatggcttctgcctaataaagaatgttcagctcaacttcctgattaatt
tcacttatttcatttttttgtccaggtgtgtaagaaggttcctgaggctctacagatagggagcacttgtttattttacaaagagtaca
tgggaaaagagaaaagcaagggaaccgtacaaggcattaatgggtgacacttctacctccaaagagcagaaattatcaagaactcttga
tacaaagataatactggcactgcagaggttctagggaagacctcaaccctaagacatagcctcaagggtaatgctacgattaaactcca
acaattactgagaaaataatgtgctcaattaaaggcataatgattactcaagacaatgttatgttgtctttcttcctccttcctttgcc
tgcacattgtagcccataatactataccccatcaagtgttcctgctccaagaaatagcttcctcctcttacttgccccagaacatctct
gtaaagaatttcctcttatcttcccatatttcagtcaagattcattgctcacgtattacttgtgacctctcttgaccccagccacaata
aacttctctatactacccaaaaaatctttccaaaccctcccccacaccattttttatatttttatatttttcttatttatttcatgcac
acacacacactccgtgctttataagcaattctgcctattctctaccttcttacatgcctactgtgcctcatattaaattcatcaatggg
cagaaagaaaatatttattcaagaaaacagtgaatgaatgaacgaatgagtaaatgagtaaatgaaggaatgattattccttgctttag
aacttctggaattagaggacaatattaataataccatcgcacagtgtttctttgttgttaatgctacaacatacaaagaggaagcatgc
agtaaacaaccgaacagttatttcctttctgatcataggagtaatatttttttccttgagcaccatttttgccataggtaaaattagaa
ggatttttagaactttctcagttgtatacatttttaaaaatctgtattatatgcatgttgattaattttaaacttacttgaatacctaa
acagaatctgttgtttccttgtgtttgaaagtgctttcacagtaactctgtctgtactgccagaatatactgacaatgtgttatagtta
actgttttgatcacaacattttgaattgactggcagcagaagctcttttatatccatgtgttttccttaagtcattatacatagtaggc
actgagaactctttatatctgaataagatatttaggaaccactggtttacatatcagaagcagagctactcagggcattttggggaaga
tcactttcacattcctgagcatagggaagttctcataagagtaagatattaaaaggagatacttgtgtggtattcgaaagacagtaaga
gagattgtagaccttatgatcttgatagggaaaacaaactacattcctttctccaaaagtcaaaaaaaaagagcaaatatagcttacta
taccttctattcctacaccattagaagtagtcagtgagtctaggcaagatgttggccctaaaaatccaaataccagagaattcatgaga
acatcacctggatgggacatgtgccgagcacacacaattactatatgctaggcattgctatcttcatattgaagatgaggaggtcaaga
gatgaaaaaagacttggcaccttgttgttatattaaaattatttgttagagtagagcttttgtaagagtctaggagtgtgggagctaaa
tgatgatacacatggacacaaaaaatagatcaacagacacccaggcctacttgagggttgagggtgggaagagggagacgatgaaaaag
aacctattgggtattaagttcatcactgagtgatgaaataatctgtacatcaagacccagtgatatgcaatttacctatataacttgta
catgtacccccaaatttaaaatgaaagttaaaacaaagtataggaatggaattaattcctcaagatttggctttaattttatttgataa
tttatcaaatggttgtttttcttttctcactatggcgttgctttataaactatgttcagtatgtctgaatgaaagggtgtgtgtgtgtg
tgaaagagagggagagaggaagggaagagaggacgtaataatgtgaatttgagttcatgaaaatttttcaataaaataatttaatgtca
ggagaattaagcctaatagtctcctaaatcatccatctcttgagcttcagagcagtcctctgaattaatgcctacatgtttgtaaaggg
tgttcagactgaagccaagattctacctctaaagagatgcaatctcaaatttatctgaagactgtacctctgctctccataaattgaca
ccatggcccacttaatgaggttaaaaaaaagctaattctgaatgaaaatctgagcccagtggaggaaatattaatgaacaaggtgcaga
ctgaaatataaatttttctgtaataattatgcatatactttagcaaagttctgtctatgttgactttattgctttttggtaagaaatac
aactttttaaagtgaactaaactatcctatttccaaactattttgtgtgtgtgcggtttgtttctatgggttctggttttcttggagca
tttttatttcattttaattaattaattctgagagctgctgagttgtgtttactgagagattgtgtatctgcgagagaagtctgtagcaa
gtagctagactgtgcttgacctaggaacatatacagtagattgctaaaatgtctcacttggggaattttagactaaacagtagagcatg
tataaaaatactctagtcaagtgctgcttttgaaacaaatgataaaaccacactcccatagatgagtgtcatgattttcatggaggaag
ttaatattcatcctctaagtatacccagactagggccattctgatataaaacattaggacttaagaaagattaatagactggagtaaag
gaaatggacctctgtctctctcgctgtctcttttttgaggacttgtgtgtgtgtgtgtgtgtgtgtgtgtgtgtgtgttgtggtcagtg
gggctggaataaaagtagaatagacctgcacctgctgtggcatccattcacagagtagaagcaagctcacaatagtgaagatgtcagta
agcttgaatagtttttcaggaactttgaatgctgatttagatttgaaactgaggctctgaccataaccaaatttgcactatttattgct
tcttgaaacttatttgcctggtatgcctgggcttttgatggtcttagtatagcttgcagccttgtccctgcagggtattatgggtaata
gaaagaaaagtctgcgttacactctagtcacactaagtaactaccattggaaaagcaacccctgccttgaagccaggatgatggtatct
gcagcagttgccaacacaagagaaggatccatagttcatcatttaaaaaagaaaacaaaatagaaaaaggaaaactatttctgagcata
agaagttgtagggtaagtctttaagaaggtgacaatttctgccaatcaggatttcaaagctcttgctttgacaattttggtctttcaga
atactataaatataacctatattataatttcataaagtctgtgcattttctttgacccaggatatttgcaaaagacatattcaaacttc
cgcagaacactttatttcacatatacatgcctcttatatcagggatgtgaaacagggtcttgaaaactgtctaaatctaaaacaatgct
aatgcaggtttaaatttaataaaataaaatccaaaatctaacagccaagtcaaatctgcatgttttaacatttaaaatattttaaagac
gtcttttcccaggattcaacatgtgaaatcttttctcagggatacacgtgtgcctagatcctcattgctttagttttttacagaggaat
gaatataaaaagaaaatacttaaattttatccctcttacctctataatcatacataggcataattttttaacctaggctccagatagcc
atagaagaaccaaacactttctgcgtgtgtgagaataatcagagtgagattttttcacaagtacctgatgagggttgagacaggtagaa
aaagtgagagatctctatttatttagcaataatagagaaagcatttaagagaataaagcaatggaaataagaaatttgtaaatttcctt
ctgataactagaaatagaggatccagtttcttttggttaacctaaattttatttcattttattgttttattttattttattttatttta
ttttgtgtaatcgtagtttcagagtgttagagctgaaaggaagaagtaggagaaacatgcaaagtaaaagtataacactttccttacta
aaccgacatgggtttccaggtaggggcaggattcaggatgactgacagggcccttagggaacactgagaccctacgctgacctcataaa
tgcttgctacctttgctgttttaattacatcttttaatagcaggaagcagaactctgcacttcaaaagtttttcctcacctgaggagtt
aatttagtacaaggggaaaaagtacagggggatgggagaaaggcgatcacgttgggaagctatagagaaagaagagtaaattttagtaa
aggaggtttaaacaaacaaaatataaagagaaataggaacttgaatcaaggaaatgattttaaaacgcagtattcttagtggactagag
gaaaaaaataatctgagccaagtagaagaccttttcccctcctacccctactttctaagtcacagaggctttttgttcccccagacact
cttgcagattagtccaggcagaaacagttagatgtccccagttaacctcctatttgacaccactgattaccccattgatagtcacactt
tgggttgtaagtgactttttatttatttgtatttttgactgcattaagaggtctctagttttttatctcttgtttcccaaaacctaata
agtaactaatgcacagagcacattgatttgtatttattctatttttagacataatttattagcatgcatgagcaaattaagaaaaacaa
caacaaatgaatgcatatatatgtatatgtatgtgtgtatatatacacacatatatatatatattttttcttttcttaccagaaggttt
taatccaaataaggagaagatatgcttagaaccgaggtagagttttcatccattctgtcctgtaagtattttgcatattctggagacgc
aggaagagatccatctacatatcccaaagctgaattatggtagacaaaactcttccacttttagtgcatcaacttcttatttgtgtaat
aagaaaattgggaaaacgatcttcaatatgcttaccaagctgtgattccaaatattacgtaaatacacttgcaaaggaggatgttttta
gtagcaatttgtactgatggtatggggccaagagatatatcttagagggagggctgagggtttgaagtccaactcctaagccagtgcca
gaagagccaaggacaggtacggctgtcatcacttagacctcaccctgtggagccacaccctagggttggccaatctactcccaggagca
gggagggcaggagccagggctgggcataaaagtcagggcagagccatctattgcttacatttgcttctgacacaactgtgttcactagc
aacctcaaacagacaccatggtgcatctgactcctgaggagaagtctgccgttactgccctgtggggcaaggtgaacgtggatgaagtt
ggtggtgaggccctgggcaggttggtatcaaggttacaagacaggtttaaggagaccaatagaaactgggcatgtggagacagagaaga
ctcttgggtttctgataggcactgactctctctgcctattggtctattttcccacccttaggctgctggtggtctacccttggacccag
aggttctttgagtcctttggggatctgtccactcctgatgctgttatgggcaaccctaaggtgaaggctcatggcaagaaagtgctcgg
tgcctttagtgatggcctggctcacctggacaacctcaagggcacctttgccacactgagtgagctgcactgtgacaagctgcacgtgg
atcctgagaacttcagggtgagtctatgggacgcttgatgttttctttccccttcttttctatggttaagttcatgtcataggaagggg
ataagtaacagggtacagtttagaatgggaaacagacgaatgattgcatcagtgtggaagtctcaggatcgttttagtttcttttattt
gctgttcataacaattgttttcttttgtttaattcttgctttctttttttttcttctccgcaatttttactattatacttaatgcctta
acattgtgtataacaaaaggaaatatctctgagatacattaagtaacttaaaaaaaaactttacacagtctgcctagtacattactatt
tggaatatatgtgtgcttatttgcatattcataatctccctactttattttcttttatttttaattgatacataatcattatacatatt
tatgggttaaagtgtaatgttttaatatgtgtacacatattgaccaaatcagggtaattttgcatttgtaattttaaaaaatgctttct
tcttttaatatacttttttgtttatcttatttctaatactttccctaatctctttctttcagggcaataatgatacaatgtatcatgcc
tctttgcaccattctaaagaataacagtgataatttctgggttaaggcaatagcaatatctctgcatataaatatttctgcatataaat
tgtaactgatgtaagaggtttcatattgctaatagcagctacaatccagctaccattctgcttttattttatggttgggataaggctgg
attattctgagtccaagctaggcccttttgctaatcatgttcatacctcttatcttcctcccacagctcctgggcaacgtgctggtctg
tgtgctggcccatcactttggcaaagaattcaccccaccagtgcaggctgcctatcagaaagtggtggctggtgtggctaatgccctgg
cccacaagtatcactaagctcgctttcttgctgtccaatttctattaaaggttcctttgttccctaagtccaactactaaactggggga
tattatgaagggccttgagcatctggattctgcctaataaaaaacatttattttcattgcaatgatgtatttaaattatttctgaatat
tttactaaaaagggaatgtgggaggtcagtgcatttaaaacataaagaaatgaagagctagttcaaaccttgggaaaatacactatatc
ttaaactccatgaaagaaggtgaggctgcaaacagctaatgcacattggcaacagcccctgatgcatatgccttattcatccctcagaa
aaggattcaagtagaggcttgatttggaggttaaagttttgctatgctgtattttacattacttattgttttagctgtcctcatgaatg
tcttttcactacccatttgcttatcctgcatctctcagccttgactccactcagttctcttgcttagagataccacctttcccctgaag
tgttccttccatgttttacggcgagatggtttctcctcgcctggccactcagccttagttgtctctgttgtcttatagaggtctacttg
aagaaggaaaaacaggggtcatggtttgactgtcctgtgagcccttcttccctgcctcccccactcacagtgacccggaatctgcagtg
ctagtctcccggaactatcactctttcacagtctgctttggaaggactgggcttagtatgaaaagttaggactgagaagaatttgaaag
gcggctttttgtagcttgatattcactactgtcttattaccctgtcataggcccaccccaaatggaagtcccattcttcctcaggatgt
ttaagattagcattcaggaagagatcagaggtctgctggctcccttatcatgtcccttatggtgcttctggctctgcagttattagcat
agtgttaccatcaaccaccttaacttcatttttcttattcaatacctaggtaggtagatgctagattctggaaataaaatatgagtctc
aagtggtccttgtcctctctcccagtcaaattctgaatctagttggcaagattctgaaatcaaggcatataatcagtaataagtgatga
tagaagggtatatagaagaattttattatatgagagggtgaaaccctcaaaatgaaatgaaatcagacccttgtcttacaccataaaca
aaaataaatttgaatgggttaaagaattaaactaagacctaaaaccataaaaatttttaaagaaatcaaaagaagaaaattctaatatt
cacgttgcagccgttttttgaatttgatatgagaagcaaaggcaacaaaaggaaaaataaagaagtgaggctacatcaaactaaaaaat
ttccacacaaaaaacaaaacaatgaacaaatgaaaggtgaaccatgaaatggcatatttgcaaaccaaatatttcttaaatattttggt
taatatccaaaatatataagaaacacagatgattcaataacaaacaaaaaattaaaaataggaaaataaaaaaattaaaaagaagaaaa
tcctgccatttatggcagaattgatgaacctggaggatgtaaaactaagaaaaataagcctgacacaaaaagacaaatactacacaacc
ttgctcatatgtgaaacataaaaaagtcactctcatggaaacagacagtagaggtatggtttccaggggttgggggtgggagaatcagg
aaactattactcaaagggtataaaatttcagttatgtgggatgaataaattctagatatctaatgtacagcatcgtgactgtagttaat
tgtactgtaagtatatttaaaatttgcaaagagagtagatttttttttttttttagatggagttttgctcttgttgtccaggctggagt
gcaatggcaagatcttggctcactgcaacctccgcctcctgggttcaagcaaatctcctgcctcagcctcccgagtagctgggattaca
ggcatgcgacaccatgcccagctaattttgtatttttagtagagacggggtttctccatgttggtcaggctgatccgcctgcctcggcc
acccaaagggctgggattacaggcgtgagccaccgggcctggccgagagtagatcttaaaagcatttaccacaagaaaaaggtaactat
gtgagataatgggtatgttaattagcttgattgtggtaatcatttcacaaggtatacatatattaaaacatcatgttgtacaccttaaa
tatatacaatttttatttgtgaatgatacctcaataaagttgaagaataataaaaaagaatagacatcacatgaattaaaaaactaaaa
aataaaaaaatgcatcttgatgattagaattgcattcttgatttttcagatacaaatatccatttgactgtttactcttttccaaaaca
atacaataaattttagcactttatcttcattttccccttcccaatctataattatatatatatatattttagatattttgtatagtttt
actccctagattttctagtgttattattaaatagtgaagaaatgtttacacttatgtacaaaatgttttgcatgcttttcttcatttct
aacattctctctaagtttattctatttttttctgattatccttaatattatctctttctgctggaaatacattgttacttttggtttat
ctaaaaatggcttcattttcttcattctaaaatcatgttaaattaataccactcatgtgtaagtaagatagtggaataaatagaaatcc
aaaaactaaatctcactaaaatataataatgtgatatataaaaatatagcttttaaatttagcttggaaataaaaaacaaacagtaatt
gaacaactatactttttgaaaagagtaaagtgaaatgcttaactgcatataccacaatcgattacacaattaggtgtgaaggtaaaatt
cagtcacgaaaaaactagaataaaaatatgggaagacatgtatataatcttagagataacactgttatttaattatcaacccaaagtag
aaactatcaagggagaaataaattcagtcaacaataaaagcatttaagaagttattctaggctgggagcggtggctcacacctgcaatt
gcagcactttgggaggcctagacaggcggatcacgacgtcaggagttcaagatcagcctggccaacatagtgaaacctcatcgctacta
aaaatataaaaacttagcctggcgtggtggcaggcatgtgtaatcccagcaatttgggaggctgaggcaggagaatcgcttgatcctgg
gaggcagaggttgcagtgagccaagattgtgccactgcattccagcccaggtgacagcatgagactccgtcacaaaaaaaaaagaaaaa
aaaaaggggggggggagcggtggagccaagatgaccgaataggaacagctccagtctatagctcccatcgtgagtgacgcagaagacgg
gtgatttctgcatttccaactgaggtaccaggttcatctcacagggaagtgccaggcagtgggtgcaggacagtaggtgcagtgcactg
tgcatgagccaaagcagggcgaggcatcacctcacccgggaagcacaaggggtcagggaattccctttcctagtcaaagaaaagggtga
cagatggcacctggaaaatcgggtcactcccgccctaatactgcgctcttccaacaagcttaacaaatggcacaccaggagattatatc
ccatgcctggctcagagggtcctacgcccatggagcctcgctcattgctagcacagcagtctgaggtcaaactgcaaggtggcagtgag
gctgggggaggggtgcccaccattgtccaggcttgagcaggtaaacaaagccgcctggaagctcgaactgggtggagcccaccacagct
caaggaggcctgcctgcctctgtaggctccacctctaggggcagggcacagacaaacaaaagacaacaagaacctctgcagacttaaat
gtccctgtctgacagctttgaagagagtagtggttctcccagcacatagcttcagatctgagaacaggcagactgcctcctcaagtggg
tccctgacccccgagtagcctaactgggaggcatcccccagtaggggcagactgacacctcacatggctggtactcctctaagacaaaa
cttccagaggaatgatcaggcagcagcatttgcggttcaccaatatccactgttctgcagccaccgctgttgatacccaggaaaacagc
ttctggagtggacctccagtaaactccaacagacctgcagctgagggtcctgactgttagaaggaaaactaacaaacagaaaggacatc
cacaccaaaaacccatctgtacatcgccatcatcaaagaccaaaggtagataaaaccataaagatggggaaaaagcagagcagaaaaac
tggacactctaaaaatgagagtgcctctcctcctccaaagtaacgcagctcctcaccagcaatggaacaaagctgggcagagaatgact
ttgacgagttgagagaggaaggcttcagaagatcaaactactccaagctaaaggaggaagttcgaacaaacggcaaagaagtaaaaaac
tttgaaaaaaaattagatgaatggataactagaataaccaatgcacagaagtccttaaaggacctgatggagctgaaaaccaaggcagg
agaactacgtgacaaatacacaagcctcagtaaccgatgagatcaactggaagaaagggtatcaatgacgaaagatgaaatgaatgaaa
tgaagcatgaagagaagtttagagaaaaaagaataaaaagaaacgaacaaagcctccaagaaatatgggactatgtgaaaagaccaaat
ctacatctaattggtgtagctgaaagtgatggggagaatggaaccaagttggaaaacactctgcaggatattatccaggagaacttccc
caatctagcaaggcaagcccaaattcacattcaggaaatacagagaacgccacaaagatactcctagagaaaagcaactccaagacaca
taactgtcagattcaccaaagttgaaatgaaggaaaaaatgttaagggcagccagagagaaaggtcgggttacccacaaagggaagccc
atcagactaacagctgatctatcggcagaaactctacaagccagaagaaagtgggggccaatattcaacattgttaaagaaaagaattt
tcaacccagaatttcatatccagccaaactaagcttcataagtgaaggagaaataaaatcctttacagacaagcaaatgctgagagatt
ttgtcaccaccaggcctgccctacaagagctcctgaaggaagcactaaacatggaaaggaacaactagtatcagccactgcaaaaacat
gccaaattgtaaagaccatcaaggctaggaagaaactgcatcaacgagcaaaataaccagctaacatcataatgacaggatcaaattca
tacataacaatactcaccttaaatgtaaataggctaaatgctccaattaaaagacacagactggcaaattggataaggagtcaagaccc
atctgtgttctgtattcaggaaacccatctcacgtgcagagacacacataggctcgaaataaaaggatggaggaatatctaccaagcaa
atggaaaacaaaaaaaggcaggggttgcaatcctagtctctgataaaacagattttaaaccaacaaagatcaaaagagacaaagaaggc
cattacataatggcaaagggatctattcaagaagaagaactaactatactaaatatatatgcacccaatacaggagcacccagattcat
aaaacaagtcctgagtgacctacaaagagacttagatgcccacacaataataatgggagactttaacaccccactgtcaacattagaca
gatcaacgagacagaaagttaacaaggatatccaggaattggactcagctctgcaccaagcagacctaatagacatctacagaactctc
caccccaaatcaacagaatatacattcttttcagcaccacaccacacctattccaaaactgaccacatagttggaagtaaagctctcct
cagcaaatgtaaaagaacagaaactataacaaactgtctctcagaccacagtgcaatcaaactagaactcaggattaagaaactcactc
aaaaccactcagctacatggaaactgaacagcctgctcctgaatgactactgggtacataacaaaatgaaggcagaaataaagatgttc
tttgaaaccaacgagaacaaagacacaacacaccagaatctctgagacacattcaaagcagtgtgtagagggaaatttatagcactaaa
tgcccacaagggaaagcaggaaagatctaaaattgacaccctaacatcacaattaaaaaactagagaagcaggagcaaacacattcaaa
agctaacagaagacaagaaataactaagatcagagcagaagtgaaggacatagagacacaaaaaaacccttcaaaaaaatcaatgaatc
cagaagctgtttttttgaaaagatcaacaaaattgatagactgctagcaagactaataaagaagaaaagagagaagaatcaaatagacg
caataaaaaatgacacggggtatcaccactgatcccacagaaatacaaactaccgtcagagaatactataaacacctctacgcaaataa
actagaaaatctagaagaaatggataaattcctcgacacatacactctgccaagactaaaccaggaagaagttgtatctctgaatagac
caataacaggctctgaaattgaggcaataattaatagcttatcaaccaaaaaaagtccgggaccagtaggattcatagccgaattctac
cagaggtacaaggaggagctggtaccattccttctgaaactattccaatcaatagaaaaagagggaatcctccctaactcattttatga
ggccagcatcatcctgataccaaagcctgacagagacacaacaaaaaaagagaatgttacaccaatatccttgatgaacattgatgcaa
aaatcctcaataaaatactggcaaactgatccaccatgatcaagtgggcttcatccctgccatgcaaggctggttcaacatacgaaaat
caataaacataatccagcatataaacagaaccaaagacacaaaccatatgattatctcaatagatgcagaaaaggcctttgacaaaatt
caacaacgcttcatgctaaaaactctcaataaattaggtattgatgggacatatctcaaaataataagagctatctatgacaaacccac
agccaatatcatactgagtggacaaaaactggaagcattccctttgaaaactggcacaaggcagggatgccctctctcaccactcctat
tcaacatagtgttgtaagttctggccagggcaatcaggcaggagaaggaaataaagggcattcaattaggaaaagaggaagtgaaattg
tccctgtttgcagatgacatgattgtatatctagaaaaccccattgtctcagcccaaaatctccttaagctgataagcaacttcagcaa
agtctcaggatataaaatcagtgtgcaaaaatcacaagtattcctatgcaccaataacagacaaacagagagccaaatcatgagtgaac
tcccattcacaattgcttcaaagagaataaaatacctaggaatccaacttacaagggatgtgaaggacctcttcaaggagaactacaaa
ccactgctcaatgaaataaaagaggatacaaacaaatggaagaacattccatgctcatgggtaggaagaatcaatatcgtgaaaatggt
catactgcccaaggtaatttatagattcaatgccatccccatcaagctaccaatgactttcttcacagaactggaaaaaactactttaa
agttcatatggaaccaaaaaagagcccacatcaccaaggcaatcctaagccaaaagaacaaagctggaggcatcacgctacctgacttc
aaactatactacaatgctacggtaaccaaaacagcatggtactggtaccaaaacagagatctagaccaatggaacagaacagagccctc
agaaataatgccgcatatctacaactatctgatctttgacaaacctgagagaaacaagcaatggggaaaggattccctatttaataaat
ggtgctgggaaaactggctagccatatgtagaaagctgaaactggatcccttccttacaccttatacaaaaattaattcaagatggatt
aaagacttacatgttagacctaaaaccataaaaaccctagaaaaaaacctaggcaataccattcaggacataggcatgggcaaggactt
catgtctaaaacaccaaaagcaatggcaacaaaagacaaaatggacaaacgggatctaattaaactaaagagcttctgcacagctaaag
aaactaccatcagagtgaacaggcaacctacaaaatgggagaaaatttttgcaatctactcatctgacaaagggctaatatccagaatc
tacaatgaactcaaacaaatttacaagaaaaaacaaacaaccccatcaaaaagtgggcaaaggatatgaacagacacttcgcaaaagaa
gacatttatgtaatcaaaaaacacatgaaaaaatgctcatcatcactagccatcagagaaatgcaaatcaaaaccacaatgagatacca
tctcacaccagttagaatggcgatcattaaaaagtcaggaaacaacaggtgctggagaggatgtggagaaacaggaacaacttttacac
tgttggtgggactgtaaactagttcaaccattgcggaagtcagtgtggcaattcctcaggaatctagaactagaaataccatttgaccc
agccatcccattactgggtacatacccaaaggattataaatcatgctgctataaagacacatgcacacgtatgtttattgcagcactat
tcacaatagcaaagacttggaaccaacccaaatgtccaacaacgatagactggattaagaaaatgtggcacatatacaccatggaatac
tatgcagccataaaaaatgatgagttcatgtcctttgtagggacatggatgaagctggaaactatcattctcagcaaactatcacaagg
agaataaaccaaacaccgcatgttctcactcataggtgggaattgaacaatgagaacacatggacacatgaagaggaacatcacactct
ggggactgttatggggtggggggcaggggcagggatagcactaggagatatacctaatgctaaatgacgagttaatgggtgcagcacac
caacatggcacatgtatacatatataacaaacctgcatgttgtgcacatgtaccctaaaacttgaagtataataataaaaaaaagttat
cctattaaaactgatctcacacatccgtagagccattatcaagtctttctctttgaaatagacagaaatttagtgttttctcagtcagt
taac

Five 5′ hypersensitive site (HS) sites (HS1-HS5) and one 3′ HS site have been identified in the human β-globin LCR (Stamatoyannopoulos et al., (2001)). The 5′ HSs 1-4 are Dnase I hypersensitive sites. The HS2 and HS3 elements are the most powerful single elements within the LCR (Ellis et al., EMBO J. (1996), 15:562-568; Collis et al., EMBO J. (1990) 9:233-240), as corroborated by many groups. Deleting HS2 in the context of BYAC in transgenic mice severely affects HS site formation as well as expression of all of the human β-globin genes at every developmental stage (Bungert et al., Mol. Cell Biol. (1999); 19:3062-3072). It was reported that deletion of HS2 minimally reduced the expression of the embryonic cy and βhi globin genes in yolk sac-derived erythrocytes (Ley et al., Ann. N.Y. Acad. Sci. (1998); 850:45-53; Hug et al., Mol. Cell Biol. (1996); 26:2906-2912). HS2 functions primarily as an enhancer.

In certain embodiments, the β-globin LCR comprises a HS2 region. In non-limiting example, the β-globin LCR comprises a HS2 region, a HS3 region, and a HS4 region. In certain embodiments, the HS2 region, HS3 region and HS4 region within the β-globin LCR are contiguous. In certain embodiments, the β-globin LCR consists essentially of a HS2 region, a HS3 region and a HS4 region. In another embodiment, the β-globin LCR comprises two introduced GATA-1 binding sites at the junction between the HS3 region and the HS4 region. The HS3 region can lie between the HS2 region and the HS4 region. The length and the sequence of the HS2 region can vary. The HS2 region can have a length of from about 400 bp to about 1000 bp, e.g., from about 400 bp to about 500 bp, from about 500 bp to about 600 bp, from about 600 bp to about 700 bp, from about 700 bp to about 800 bp, from about 800 bp to about 850 bp, from about 800 bp to about 820 bp, from about 820 bp to about 830 bp, from about 830 bp to about 840 bp, from about 840 bp to about 850 bp, from about 850 bp to about 900 bp, from about 800 bp to about 900 bp, or from about 900 bp to about 1000 bp.

In certain embodiments, the HS2 region has a length of from about 850 bp to about 900 bp. In certain non-limiting embodiments, the HS2 region has a length of about 860 bp. In certain non-limiting embodiments, the HS2 region comprises or has the nucleotide sequence set forth in SEQ ID NO:9, which is provided below:

[SEQ ID NO: 9]
GTATATGTGTATATATATATATATATATTCAGGAAATAATATATTCTAGA
ATATGTCACATTCTGTCTCAGGCATCCATTTTCTTTATGATGCCGTTTGA
GGTGGAGTTTTAGTCAGGTGGTCAGCTTCTCCttttttttGCCATCTGCC
CTGTAAGCATCCTGCTGGGGACCCAGATAGGAGTCATCACTCTAGGCTGA
GAACATCTGGGCACACACCCTAAGCCTCAGCATGACTCATCATGACTCAG
CATTGCTGTGCTTGAGCCAGAAGGTTTGCTTAGAAGGTTACACAGAACCA
GAAGGCGGGGGTGGGGCACTGACCCCGACAGGGGCCTGGCCAGAACTGCT
CATGCTTGGACTATGGGAGGTCACTAATGGAGACACACAGAAATGTAACA
GGAACTAAGGAAAAACTGAAGCTTATTTAATCAGAGATGAGATGCTGGAA
GGGATAGAGGGAGCTGAGCTTGTAAAAAGTATAGTAATCATTCAGCAAAT
GGTTTTGAAGCACCTGCTGGATGCTAAACACTATTTTCAGTGCTTGAATC
ATAAATAAGAATAAAACATGTATCTTATTCCCCACAAGAGTCCAAGTAAA
AAATAACAGTTAATTATAATGTGCTCTGTCCCCCAGGCTGGAGTGCAGTG
GCACGATCTCAGCTCACTGCAACCTCCGCCTCCCGGGTTCAAGCAATTCT
CCTGCCTCAGCCACCCTAATAGCTGGGATTACAGGTGCACACCACCATGC
CAGGCTAATTTTTGTACTTTTTGTAGAGGCAGGGTATCACCATGTTGTCC
AAGATGGTCTTGAACTCCTGAGCTCCAAGCAGTCCACCCACCTCAGCCTC
CCAAAGTGCT

In certain embodiments, the HS2 region has a length of from about 800 bp to about 850 bp. In certain embodiments, the HS2 region has a length of about 840 bp. In certain embodiments, the HS2 region has a length of about 820 bp. In certain embodiments, the HS2 region has a length of about 816 bp.

In certain non-limiting embodiments, the HS2 region comprises or has the nucleotide sequence that is a consecutive portion of SEQ ID NO:9. In certain embodiments, the consecutive portion of SEQ ID NO: 9 is at least about 600 bp, or at least about 700 bp, or at least about 800 bp, or at least about 820 bp, and up to about 860 bp in length. Alternatively or additionally, in non-limiting various embodiments, the HS2 region comprises or has nucleotides 1 to 860, 20 to 860, 30 to 860, 40 to 860, 45 to 860, 50 to 860, 100 to 860, or 200 to 860 of SEQ ID NO: 9. In certain embodiments, the HS2 region comprises or has nucleotides 45 to 860 of SEQ ID NO: 9.

In certain embodiments, the HS2 region comprises or has a nucleotide sequence that is at least about 80%, at least about 85%, at least about 90%, at least about 99%, or at least about 100% homologous to the nucleotide sequence set forth in SEQ ID NO:9 or a consecutive portion thereof. In certain embodiments, the HS2 region comprises or has a nucleotide sequence that is at least about 99% homologous to the nucleotide sequence set forth in SEQ ID NO:9. In certain embodiments, the HS2 region comprises or has a nucleotide sequence that is about 100% homologous to nucleotides 45 to 860 of SEQ ID NO: 9, e.g., the HS2 region has a nucleotide sequence that includes up to 5, up to 4, up to 3, up to 2, or up to 1 mutations of nucleotides 45 to 860 of SEQ ID NO: 9. In certain embodiments, the HS2 region comprises or has the nucleotide sequence set forth in SEQ ID NO: 33, which is provided below.

(SEQ ID NO: 33)
TCTAGAATATGTCACATTCTGTCTCAGGCATCCATTTTCTTTATGATGCC
GTTTGAGGTGGAGTTTTAGTCAGGTGGTCAGCTTCTCCttttttttGCCA
TCTGCCCTGTAAGCATCCTGCTGGGGACCCAGATAGGAGTCATCACTCTA
GGCTGAGAACATCTGGGCACACACCCTAAGCCTCAGCATGACTCATCATG
ACTCAGCATTGCTGTGCTTGAGCCAGAAGGTTTGCTTAGAAGGTTACACA
GAACCAGAAGGCGGGGGTGGGGCACTGACCCCGACAGGGGCCTGGCCAGA
ACTGCTCATGCTTGGACTATGGGAGGTCACTAATGGAGACACACAGAAAT
GTAACAGGAACTAAGGAAAAACTGAAGCTTATTTAATCAGAGATGAGATG
CTGGAAGGGATAGAGGGAGCTGAGCTTGTAAAAAGTATAGTAATCATTCA
GCAAATGGTTTTGAAGCACCTGCTGGATGCTAAACACTATTTTCAGTGCT
TGAATCATAAATAAGAACAAAACATGTATCTTATTCCCCACAAGAGTCCA
AGTAAAAAATAACAGTTAATTATAATGTGCTCTGTCCCCCAGGCTGGAGT
GCAGTGGCACGATCTCAGCTCACTGCAACCTCCGCCTCCCGGGTTCAAGC
AATTCTCCTGCCTCAGCCACCCTAATAGCTGGGATTACAGGTGCACACCA
CCATGCCAGGCTAATTTTTGTACTTTTTGTAGAGGCAGGGTATCACCATG
TTGTCCAAGATGGTCTTGAACTCCTGAGCTCCAAGCAGTCCACCCACCTC
AGCCTCCCAAAGTGCT.

In certain embodiments, the HS2 region comprises or has a nucleotide sequence that is a modification of the nucleotide sequence set forth in SEQ ID NO: 9 or a consecutive portion thereof. Non-limiting modifications include deletion, mutations, additions, or combinations thereof. In certain embodiments, the modification comprises one or more mutation, e.g., up to 5, up to 4, up to 3, up to 2, or up to 1 mutations. In certain embodiments, the modification comprises one mutation. In certain embodiments, the one or more mutation is located within a polyadenylation site (or polyadenylation signal motif), e.g., a polyadenylation site on SEQ ID NO: 9. In certain embodiments, the polyadenylation site has the nucleotide sequence AATAAA. In certain embodiments, the one or more mutation comprises a mismatch mutation T>C at a polyadenylation site having the nucleotide sequence AATAAA, e.g., the mutated sequence is AACAAA. In certain embodiments, the polyadenylation site has the nucleotide sequence ATTAAA. In certain embodiments, the one or more mutation comprises a mismatch mutation T>C at a polyadenylation site having the nucleotide sequence ATTAAA, e.g., the mutated sequence is ATCAAA or ACTAAA. In certain embodiments, the one or more mutation comprises two mismatch mutations T>C at a polyadenylation site having the nucleotide sequence ATTAAA, e.g., the mutated sequence is ACCAAA. The inventors discovered that a modification (e.g., one or more mismatch mutation) at a polyadenylation site can preempt premature termination of RNA transcription, thereby increasing the titer of the expression cassette (e.g., expression vector) without compromising the expression of the globin gene.

In certain embodiments, the HS2 region has a length of about 650 bp (e.g., 646 bp). In certain embodiments, the HS2 region has a length of about 420 bp (e.g., 423 bp).

The length and the sequence of the HS3 region can vary. The HS3 region can have a length of from about 200 bp to about 1400 bp, e.g., from about 200 bp to about 300 bp, from about 300 bp to about 400 bp, from about 400 bp to about 500 bp, from about 500 bp to about 600 bp, from about 600 bp to about 700 bp, from about 700 bp to about 800 bp, from about 800 bp to about 900 bp, from about 900 bp to about 1000 bp, from about 1000 bp to about 1100 bp, from about 1100 bp to about 1200 bp, from about 1200 bp to about 1300 bp, or from about 1300 bp to about 1400 bp. In certain embodiments, the HS3 region has a length of about 1300 bp. In certain non-limiting embodiments, the HS3 region has a length of 1308 bp. In certain non-limiting embodiments, the HS3 region has a length of 1301 bp. In certain non-limiting embodiments, the HS3 region comprises or has the nucleotide sequence set forth in SEQ ID NO:5, which is provided below:

[SEQ ID NO: 5]
AAGCTTTCATTAAAAAAAGTCTAACCAGCTGCATTCGACTTTGACTGCAG
CAGCTGGTTAGAAGGTTCTACTGGAGGAGGGTCCCAGCCCATTGCTAAAT
TAACATCAGGCTCTGAGACTGGCAGTATATCTCTAACAGTGGTTGATGCT
ATCTTCTGGAACTTGCCTGCTACATTGAGACCACTGACCCATACATAGGA
AGCCCATAGCTCTGTCCTGAACTGTTAGGCCACTGGTCCAGAGAGTGTGC
ATCTCCTTTGATCCTCATAATAACCCTATGAGATAGACACAATTATTACT
CTTACTTTATAGATGATGATCCTGAAAACATAGGAGTCAAGGCACTTGCC
CCTAGCTGGGGGTATAGGGGAGCAGTCCCATGTAGTAGTAGAATGAAAAA
TGCTGCTATGCTGTGCCTCCCCCACCTTTCCCATGTCTGCCCTCTACTCA
TGGTCTATCTCTCCTGGCTCCTGGGAGTCATGGACTCCACCCAGCACCAC
CAACCTGACCTAACCACCTATCTGAGCCTGCCAGCCTATAACCCATCTGG
GCCCTGATAGCTGGTGGCCAGCCCTGACCCCACCCCACCCTCCCTGGAAC
CTCTGATAGACACATCTGGCACACCAGCTCGCAAAGTCACCGTGAGGGTC
TTGTGTTTGCTGAGTCAAAATTCCTTGAAATCCAAGTCCTTAGAGACTCC
TGCTCCCAAATTTACAGTCATAGACTTCTTCATGGCTGTCTCCTTTATCC
ACAGAATGATTCCTTTGCTTCATTGCCCCATCCATCTGATCCTCCTCATC
AGTGCAGCACAGGGCCCATGAGCAGTAGCTGCAGAGTCTCACATAGGTCT
GGCACTGCCTCTGACATGTCCGACCTTAGGCAAATGCTTGACTCTTCTGA
GCTCAGTCTTGTCATGGCAAAATAAAGATAATAATAGTGTTTTTTTATGG
AGTTAGCGTGAGGATGGAAAACAATAGCAAAATTGATTAGACTATAAAAG
GTCTCAACAAATAGTAGTAGATTTTATCATCCATTAATCCTTCCCTCTCC
TCTCTTACTCATCCCATCACGTATGCCTCTTAATTTTCCCTTACCTATAA
TAAGAGTTATTCCTCTTATTATATTCTTCTTATAGTGATTCTGGATATTA
AAGTGGGAATGAGGGGCAGGCCACTAACGAAGAAGATGTTTCTCAAAGAA
GCCATTCTCCCCACATAGATCATCTCAGCAGGGTTCAGGAAGATAAAGGA
GGATCAAGGTCGAAGGTAGGAACTAAGGAAGAACACTGGGCAAGTGGATC
C

In certain embodiments, the HS2 region comprises or has the nucleotide sequence that is a consecutive portion of SEQ ID NO: 5. In certain embodiments, the consecutive portion of SEQ ID NO: 5 is at least about 600 bp, or at least about 700 bp, or at least about 800 bp, at least about 900 bp, at least about 1000 bp, at least about 1100 bp, or at least about 1200 bp, and up to about 1300 bp in length.

In certain embodiments, the HS3 region comprises or has a nucleotide sequence that is at least about 80%, at least about 85%, at least about 90%, at least about 99%, or at least about 100% homologous to the nucleotide sequence set forth in SEQ ID NO: 5 or a consecutive portion thereof. In certain embodiments, the HS3 region comprises or has a nucleotide sequence that is at least about 99% homologous to the nucleotide sequence set forth in SEQ ID NO: 5. In certain embodiments, the HS3 region comprises or has a nucleotide sequence that is about 100% homologous to the nucleotide sequence set forth in SEQ ID NO: 5, e.g., the HS3 region has a nucleotide sequence that includes up to 5, up to 4, up to 3, up to 2, or up to 1 mutations of SEQ ID NO: 5. In certain embodiments, the HS3 region comprises or has the nucleotide sequence set forth in SEQ ID NO: 34, which is provided below.

(SEQ ID NO: 34)
AAGCTTTCATCaaaaaaaGTCTAACCAGCTGCATTCGACTTTGACTGCAG
CAGCTGGTTAGAAGGTTCTACTGGAGGAGGGTCCCAGCCCATTGCTAAAT
TAACATCAGGCTCTGAGACTGGCAGTATATCTCTAACAGTGGTTGATGCT
ATCTTCTGGAACTTGCCTGCTACATTGAGACCACTGACCCATACATAGGA
AGCCCATAGCTCTGTCCTGAACTGTTAGGCCACTGGTCCAGAGAGTGTGC
ATCTCCTTTGATCCTCATAATAACCCTATGAGATAGACACAATTATTACT
CTTACTTTATAGATGATGATCCTGAAAACATAGGAGTCAAGGCACTTGCC
CCTAGCTGGGGGTATAGGGGAGCAGTCCCATGTAGTAGTAGAATGAAAAA
TGCTGCTATGCTGTGCCTCCCCCACCTTTCCCATGTCTGCCCTCTACTCA
TGGTCTATCTCTCCTGGCTCCTGGGAGTCATGGACTCCACCCAGCACCAC
CAACCTGACCTAACCACCTATCTGAGCCTGCCAGCCTATAACCCATCTGG
GCCCTGATAGCTGGTGGCCAGCCCTGACCCCACCCCACCCTCCCTGGAAC
CTCTGATAGACACATCTGGCACACCAGCTCGCAAAGTCACCGTGAGGGTC
TTGTGTTTGCTGAGTCAAAATTCCTTGAAATCCAAGTCCTTAGAGACTCC
TGCTCCCAAATTTACAGTCATAGACTTCTTCATGGCTGTCTCCTTTATCC
ACAGAATGATTCCTTTGCTTCATTGCCCCATCCATCTGATCCTCCTCATC
AGTGCAGCACAGGGCCCATGAGCAGTAGCTGCAGAGTCTCACATAGGTCT
GGCACTGCCTCTGACATGTCCGACCTTAGGCAAATGCTTGACTCTTCTGA
GCTCAGTCTTGTCATGGCAAAACAAAGATAATAATAGTGtttttttATGG
AGTTAGCGTGAGGATGGAAAACAATAGCAAAATTGATTAGACTATAAAAG
GTCTCAACAAATAGTAGTAGATTTTATCATCCATTAATCCTTCCCTCTCC
TCTCTTACTCATCCCATCACGTATGCCTCTTAATTTTCCCTTACCTATAA
TAAGAGTTATTCCTCTTATTATATTCTTCTTATAGTGATTCTGGATATCA
AAGTGGGAATGAGGGGCAGGCCACTAACGAAGAAGATGTTTCTCAAAGAA
GCCATTCTCCCCACATAGATCATCTCAGCAGGGTTCAGGAAGATAAAGGA
GGATCAAGGTCGAAGGTAGGAACTAAGGAAGAACACTGGGCAAGTGGATC
C.

In certain embodiments, the HS3 region comprises or has a nucleotide sequence that is a modification of the nucleotide sequence set forth in SEQ ID NO: 5 or a consecutive portion thereof. Non-limiting modifications include deletion, mutations, additions, or combinations thereof. In certain embodiments, the modification comprises one or more mutation, e.g., up to 5, up to 4, up to 3, up to 2, or up to 1 mutations. In certain embodiments, the modification comprises three mutations. In certain embodiments, the one or more mutation is located within a polyadenylation site (or polyadenylation signal motif), e.g., a polyadenylation site on SEQ ID NO: 5. In certain embodiments, the polyadenylation site has the nucleotide sequence AATAAA. In certain embodiments, the one or more mutation comprises a mismatch mutation T>C at a polyadenylation site having the nucleotide sequence AATAAA, e.g., the mutated sequence is AACAAA. In certain embodiments, the polyadenylation site has the nucleotide sequence ATTAAA. In certain embodiments, the one or more mutation comprises a mismatch mutation T>C at a polyadenylation site having the nucleotide sequence ATTAAA, e.g., the mutated sequence is ATCAAA or ACTAAA. In certain embodiments, the one or more mutation comprises two mismatch mutations T>C at a polyadenylation site having the nucleotide sequence ATTAAA, e.g., the mutated sequence is ACCAAA.

A modification (e.g., one or more mismatch mutation) at a polyadenylation site can preempt premature termination of RNA transcription, thereby increasing the titer of the expression cassette (e.g., expression vector) without compromising the expression of the globin gene.

In certain embodiments, the HS3 region has a length of about 850 bp (e.g., 845 bp). In certain embodiments, the HS3 region has a length of from about 280 bp to about 290 bp (e.g., 280 bp and 287 bp).

Similarly, the length and the sequence of the HS4 region can vary. The HS4 region can have a length of from about 200 bp to about 1200 bp, e.g., from about 200 bp to about 300 bp, from about 300 bp to about 400 bp, from about 400 bp to about 500 bp, from about 500 bp to about 600 bp, from about 600 bp to about 700 bp, from about 700 bp to about 800 bp, from about 800 bp to about 900 bp, from about 900 bp to about 1000 bp, from about 1000 bp to about 1100 bp, or from about 1100 bp to about 1200 bp.

In certain embodiments, the HS4 region has a length of about 1.0 kb or more. In certain embodiments, the HS4 region has a length of about 1.1 kb. In certain embodiments, the HS4 region has a length of about 1150 bp (e.g., 1153 bp). In certain non-limiting embodiments, the HS4 region has a length of 1100 bp. In certain non-limiting embodiments, the HS4 region has a length of 1065 bp. In certain non-limiting embodiments, the HS4 region has the nucleotide sequence set forth in SEQ ID NO:6, which is provided below:

[SEQ ID NO: 6]
TGAGCCCCTTTTCCTCTAACTGAAAGAAGGAAAAAAAAAATGGAACCCAA
AATATTCTACATAGTTTCCATGTCACAGCCAGGGCTGGGCAGTCTCCTGT
TATTTCTTTTAAAATAAATATATCATTTAAATGCATAAATAAGCAAACCC
TGCTCGGGAATGGGAGGGAGAGTCTCTGGAGTCCACCCCTTCTCGGCCCT
GGCTCTGCAGATAGTGCTATCAAAGCCCTGACAGAGCCCTGCCCATTGCT
GGGCCTTGGAGTGAGTCAGCCTAGTAGAGAGGCAGGGCAAGCCATCTCAT
AGCTGCTGAGTGGGAGAGAGAAAAGGGCTCATTGTCTATAAACTCAGGTC
ATGGCTATTCTTATTCTCACACTAAGAAAAAGAATGAGATGTCTACATAT
ACCCTGCGTCCCCTCTTGTGTACTGGGGCCCCCAAGAGCTCTCTAAAAGT
GATGGCAAAGTCATTGCGCTAGATGCCATCCCATCTATTATAAACCTGCA
TTTGTCTCCACACACCAGTCATGGACAATAACCCTCCTCCCAGGTCCACG
TGCTTGTCTTTGTATAATACTCAAGTAATTTCGGAAAATGTATTCTTTCA
ATCTTGTTCTGTTATTCCTGTTTCAATGGCTTAGTAGAAAAAGTACATAC
TTGTTTTCCCATAAATTGACAATAGACAATTTCACATCAATGTCTATATG
GGTCGTTGTGTTTGCTGTGTTTGCAAAAACTCACAATAACTTTATATTGT
TACTACTCTAAGAAAGTTACAACATGGTGAATACAAGAGAAAGCTATTAC
AAGTCCAGAAAATAAAAGTTATCATCTTGAGGCCTCAGCTTTCTAGGAAT
AATATCAATATTACAAAATTTAATCTAACAATTATGAACAGCAATGAGAT
AATATGTACAAAGTACCCAGACCTATGTGGTAGAGCATCAAGGAAGCGCA
TTGCGGAGCAGTTTTTTGTTTGTTTGTTTTTGTATTCTGTTTCGTGAGGC
AAGGTTTCACTCTGCTGTCCAGGCTGGAGTGCAGTGGCAAGATCATGTCT
CACTGCAGCCTTGAC

In certain non-limiting embodiments, the HS4 region has the nucleotide sequence set forth in SEQ ID NO:7, which is provided below:

[SEQ ID NO: 7]
TGAGCCCCTTTTCCTCTAACTGAAAGAAGGAAAAAAAAAATGGAACCCAA
AATATTCTACATAGTTTCCATGTCACAGCCAGGGCTGGGCAGTCTCCTGT
TATTTCTTTTAAAATAAATATATCATTAAATGCATAAATAAGCAAACCCT
GCTCGGGAATGGGAGGGAGAGTCTCTGGAGTCCACCCCTTCTCGGCCCTG
GCTCTGCAGATAGTGCTATCAAAGCCCTGACAGAGCCCTGCCCATTGCTG
GGCCTTGGAGTGAGTCAGCCTAGTAGAGAGGCAGGGCAAGCCATCTCATA
GCTGCTGAGTGGGAGAGAGAAAAGGGCTCATTGTCTATAAACTCAGGTCA
TGGCTATTCTTATTCTCACACTAAGAAAAAGAATGAGATGTCTACATATA
CCCTGCGTCCCCTCTTGTGTACTGGGGCCCCCAAGAGCTCTCTAAAAGTG
ATGGCAAAGTCATTGCGCTAGATGCCATCCCATCTATTATAAACCTGCAT
TTGTCTCCACACACCAGTCATGGACAATAACCCTCCTCCCAGGTCCACGT
GCTTGTCTTTGTATAATACTCAAGTAATTTCGGAAAATGTATTCTTTCAA
TCTTGTTCTGTTATTCCTGTTTCAATGGCTTAGTAGAAAAAGTACATACT
TGTTTTCCCATAAATTGACAATAGACAATTTCACATCAATGTCTATATGG
GTCGTTGTGTTTGCTGTGTTTGCAAAAACTCACAATAACTTTATATTGTT
ACTACTCTAAGAAAGTTACAACATGGTGAATACAAGAGAAAGCTATTACA
AGTCCAGAAAATAAAAGTTATCATCTTGAGGCCTCAGCTTTCTAGGaATA
ATATCAATATTACAAAATTAATCTAACAATTATGAACAGCAATGAGATAA
TATGTACAAAGTACCCAGACCTATGTGGTAGAGCATCAAGGAAGCGCATT
GCGGAGCAGTTTTTTGTTTGTTTGTTTTTGTATTCTGTTTCGTGAGGCAA
GGTTTCACTCTGCTGTCCAGGCTGGAGTGCAGTGGCAAGATCATGTCTCA
CTGCAGCCTTGACAC

In certain embodiments, the HS4 region has a length of less than about 1.0 kb, less than about 900 bp, or less than about 800 bp. In certain embodiments, the HS4 region has a length of from about 700 bp to about 800 bp, e.g., from about 700 bp to about 750 bp, or from about 750 bp to about 800 bp. In certain embodiments, the HS4 region has a length of about 750 bp. In certain embodiments, the HS4 region has a length of about 754 bp.

In certain embodiments, the HS4 region comprises or has the nucleotide sequence that is a consecutive portion of SEQ ID NO: 6 or 7. In certain embodiments, the consecutive portion of SEQ ID NO: 6 or 7 is at least about 600 bp or at least about 700 bp and up to about 800 bp or up to about 900 bp, or up to about 1.0 kb in length. Alternatively or additionally, in non-limiting various embodiments, the HS4 region comprises or has nucleotides 1 to 900, 100 to 900, 100 to 1000, 110 to 900, 115 to 900, 115 to 890, 115 to 880, 115 to 870, 115 to 868, 115 to 860, 115 to 1000 of SEQ ID NO: 6 or 7. In certain embodiments, the HS4 region comprises or has nucleotides 115 to 868 of SEQ ID NO: 6.

In certain embodiments, the HS4 region comprises or consists essentially of a core sequence of the HS4 region. In certain embodiments, the HS4 region or consists essentially of the full length of a 280 bp core sequence of a human HS4 region (e.g., the 280 bp core sequence of human HS4 disclosed in Pruzina et al., Nucleic Acdis Research (1991); 19:7:1413-1419, which is incorporated by reference in its entirety).

In certain embodiments, the HS4 region does not comprise the nucleotide sequence set forth in SEQ ID NO: 50, which is provided below.

[SEQ ID NO: 50]
TGAGCCCCTTTTCCTCTAACTGAAAGAAGGAAAAAAAAAATGGAACC
CAAAATATTCTACATAGTTTCCATGTCACAGCCAGGGCTGGGCAGTC
TCCTGTTATTTCTTTTAAAA

In certain embodiments, the HS4 region does not comprise the nucleotide sequence set forth in SEQ ID NO: 51, which is provided below.

[SEQ ID NO: 51]
TTTAATCTAACAATTATGAACAGCAATGAGATAATATGTACAAAGTA
CCCAGACCTATGTGGTAGAGCATCAAGGAAGCGCATTGCGGAGCAGT
TTTTTGTTTGTTTGTTTTTGTATTCTGTTTCGTGAGGCAAGGTTTCA
CTCTGCTGTCCAGGCTGGAGTGCAGTGGCAAGATCATGTCTCACTGC
AGCCTTGAC

In certain embodiments, the HS4 region does not comprise the nucleotide sequence set forth in SEQ ID NO: 50 or the nucleotide sequence set forth in SEQ ID NO: 51.

In certain embodiments, the HS4 region comprises or has a nucleotide sequence that is at least about 80%, at least about 85%, at least about 90%, at least about 99%, or at least about 100% homologous to the nucleotide sequence set forth in SEQ ID NO: 6 or a consecutive portion thereof. In certain embodiments, the HS4 region comprises or has a nucleotide sequence that is at least about 80%, at least about 85%, at least about 90%, at least about 99%, or at least about 100% homologous to the nucleotide sequence set forth in SEQ ID NO: 7 or a consecutive portion thereof. In certain embodiments, the HS4 region comprises or has a nucleotide sequence that is at least about 99% homologous to the nucleotide sequence set forth in SEQ ID NO: 6 or 7. In certain embodiments, the HS4 region comprises or has a nucleotide sequence that is at least about 99% (e.g., about 100%) homologous to nucleotides 115 to 868 of SEQ ID NO: 6, e.g., the HS4 region has a nucleotide sequence that includes up to 5, up to 4, up to 3, up to 2, or up to 1 mutations of nucleotides 115 to 868 of SEQ ID NO: 6. In certain embodiments, the HS4 region comprises or has the nucleotide sequence set forth in SEQ ID NO: 35, which is provided below.

[SEQ ID NO: 35]
TAAATATATCATTTAAATGCATAAATAAGCAAACCCTGCTCGGGAAT
GGGAGGGAGAGTCTCTGGAGTCCACCCCTTCTCGGCCCTGGCTCTGC
AGATAGTGCTATCAAAGCCCTGACAGAGCCCTGCCCATTGCTGGGCC
TTGGAGTGAGTCAGCCTAGTAGAGAGGCAGGGCAAGCCATCTCATAG
CTGCTGAGTGGGAGAGAGAAAAGGGCTCATTGTCTATAAACTCAGGT
CATGGCTATTCTTATTCTCACACTAAGAAAAAGAATGAGATGTCTAC
ATATACCCTGCGTCCCCTCTTGTGTACTGGGGCCCCCAAGAGCTCTC
TAAAAGTGATGGCAAAGTCATTGCGCTAGATGCCATCCCATCTATTA
TAAACCTGCATTTGTCTCCACACACCAGTCATGGACAATAACCCTCC
TCCCAGGTCCACGTGCTTGTCTTTGTATAATACTCAAGTAATTTCGG
AAAATGTATTCTTTCAATCTTGTTCTGTTATTCCTGTTTCAATGGCT
TAGTAGAAAAAGTACATACTTGTTTTCCCATAAATTGACAATAGACA
ATTTCACATCAATGTCTATATGGGTCGTTGTGTTTGCTGTGTTTGCA
AAAACTCACAATAACTTTATATTGTTACTACTCTAAGAAAGTTACAA
CATGGTGAATACAAGAGAAAGCTATTACAAGTCCAGAAAACAAAAGT
TATCATCTTGAGGCCTCAGCTTTCTAGGAATAATATCAATATTACAA
AA.

In certain embodiments, the HS4 region comprises or has a nucleotide sequence that is a modification of the nucleotide sequence set forth in SEQ ID NO: 6 or a consecutive portion thereof. In certain embodiments, the HS4 region comprises or has a nucleotide sequence that is a modification of the nucleotide sequence set forth in SEQ ID NO: 7 or a consecutive portion thereof. Non-limiting modifications include deletion, mutations, additions, or combinations thereof. In certain embodiments, the modification comprises one or more mutation, e.g., up to 5, up to 4, up to 3, up to 2, or up to 1 mutations. In certain embodiments, the modification comprises one mutation. In certain embodiments, the one or more mutation is located within a polyadenylation site (or polyadenylation signal motif), e.g., a polyadenylation site on SEQ ID NO: 6 or a polyadenylation site on SEQ ID NO: 7. In certain embodiments, the polyadenylation site has the nucleotide sequence AATAAA. In certain embodiments, the one or more mutation comprises a mismatch mutation T>C at a polyadenylation site having the nucleotide sequence AATAAA, e.g., the mutated sequence is AACAAA. In certain embodiments, the polyadenylation site has the nucleotide sequence ATTAAA. In certain embodiments, the one or more mutation comprises a mismatch mutation T>C at a polyadenylation site having the nucleotide sequence ATTAAA, e.g., the mutated sequence is ATCAAA or ACTAAA. In certain embodiments, the one or more mutation comprises two mismatch mutations T>C at a polyadenylation site having the nucleotide sequence ATTAAA, e.g., the mutated sequence is ACCAAA. A modification (e.g., one or more mismatch mutation) at a polyadenylation site can preempt premature termination of RNA transcription, thereby increasing the titer of the expression cassette (e.g., expression vector) without compromising the expression of the globin gene.

In certain embodiments, the HS4 region has a length of less than about 500 bp. In certain embodiments, the HS4 region has a length of about 450 bp. In certain non-limiting embodiments, the HS4 region has a length of about 446 bp. In certain non-limiting embodiments, the HS4 region comprises or has the nucleotide sequence set forth in SEQ ID NO:8, which is provided below:

[SEQ ID NO: 8]
TGGAACCCAAAATATTCTACATAGTTTCCATGTCACAGCCAGGGCTG
GGCAGTCTCCTGTTATTTCTTTTAAAATAAATATATCATTTAAATGC
ATAAATAAGCAAACCCTGCTCGGGAATGGGAGGGAGAGTCTCTGGAG
TCCACCCCTTCTCGGCCCTGGCTCTGCAGATAGTGCTATCAAAGCCC
TGACAGAGCCCTGCCCATTGCTGGGCCTTGGAGTGAGTCAGCCTAGT
AGAGAGGCAGGGCAAGCCATCTCATAGCTGCTGAGTGGGAGAGAGAA
AAGGGCTCATTGTCTATAAACTCAGGTCATGGCTATTCTTATTCTCA
CACTAAGAAAAAGAATGAGATGTCTACATATACCCTGCGTCCCCTCT
TGTGTACTGGGGTCCCCAAGAGCTCTCTAAAAGTGATGGCAAAGTCA
TTGCGCTAGATGCCATCCCATCT

In certain embodiments, the HS4 region has a length of about 280 bp (e.g., 283 bp). In certain embodiments, the HS4 region has a length of about 240 bp (e.g., 243 bp).

In certain non-limiting embodiments, the β-globin LCR comprises or consists essentially of a HS2 region having the nucleotide sequence set forth in SEQ ID NO:9 or a consecutive portion thereof, a modification of SEQ ID NO: 9, a modification of a consecutive portion of SEQ ID NO: 9 (e.g., SEQ ID NO: 33), SEQ ID NO:20, SEQ ID NO:21; a HS3 region having the nucleotide sequence set forth in SEQ ID NO:5 or a modification of SEQ ID NO: 5 (e.g., SEQ ID NO: 34); and a HS4 region having the nucleotide sequence set forth in SEQ ID NO:6 or a consecutive portion thereof, SEQ ID NO:7 or a consecutive portion thereof, a modification of SEQ ID NO: 6, a modification of a consecutive portion of SEQ ID NO: 6 (e.g., SEQ ID NO: 35), a modification of SEQ ID NO: 7, a modification of a consecutive portion of SEQ ID NO: 7, or SEQ ID NO:8, and the β-globin LCR does not comprise a HS1 region.

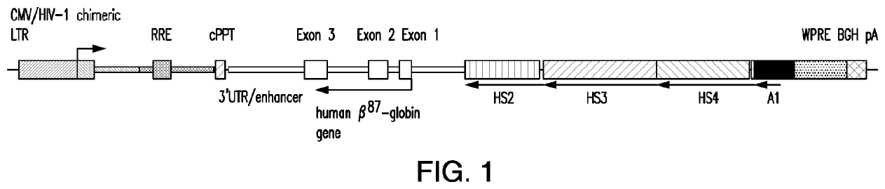

In certain non-limiting embodiments, the β-globin LCR comprises or consists essentially of a HS2 region having the nucleotide sequence set forth in SEQ ID NO:9, a HS3 region having the nucleotide sequence set forth in SEQ ID NO:5, and a HS4 region having the nucleotide sequence set forth in SEQ ID NO:7, as shown in FIG. 1.

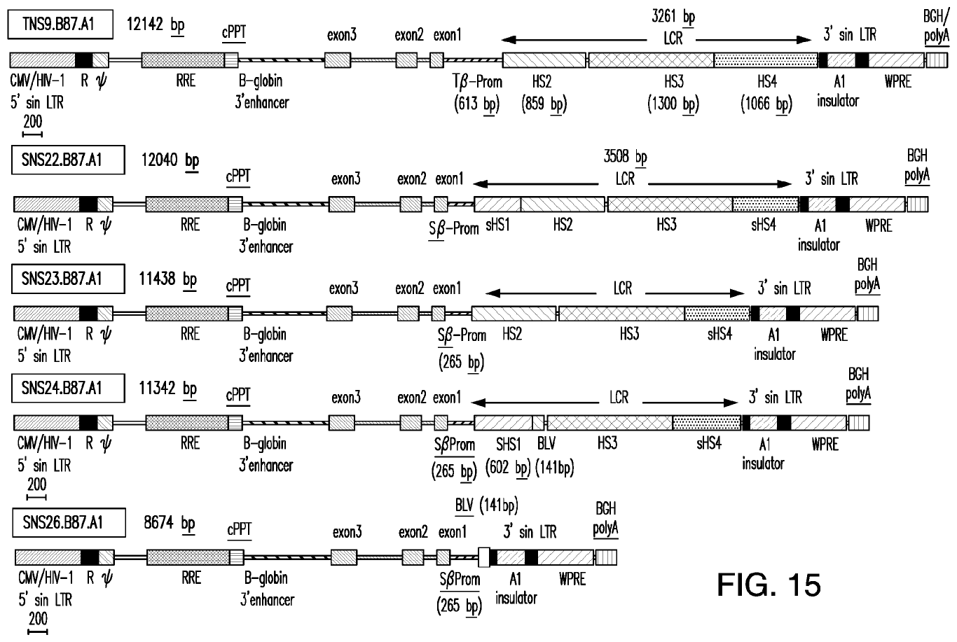

In certain non-limiting embodiments, the β-globin LCR comprises or consists essentially of a HS2 region having nucleotides 45 to 860 of SEQ ID NO: 9; a HS3 region having the nucleotide sequence set forth in SEQ ID NO: 5; and a HS4 region having nucleotides 115 to 868 of SEQ ID NO: 6, and the β-globin LCR does not comprise a HS1 region, e.g., the β-globin LCR of SNS23.B87.A1 shown in FIG. 15.

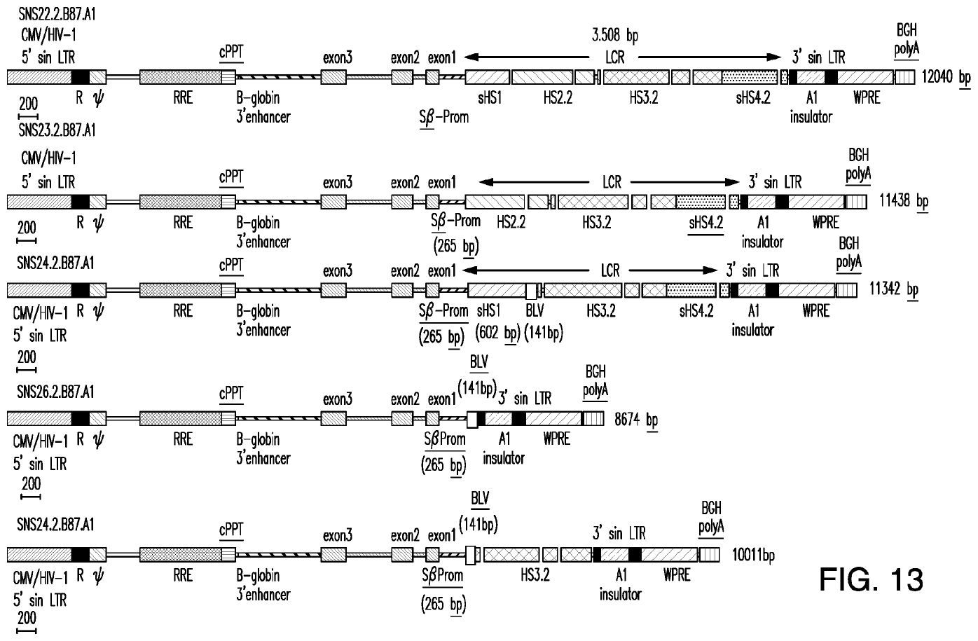

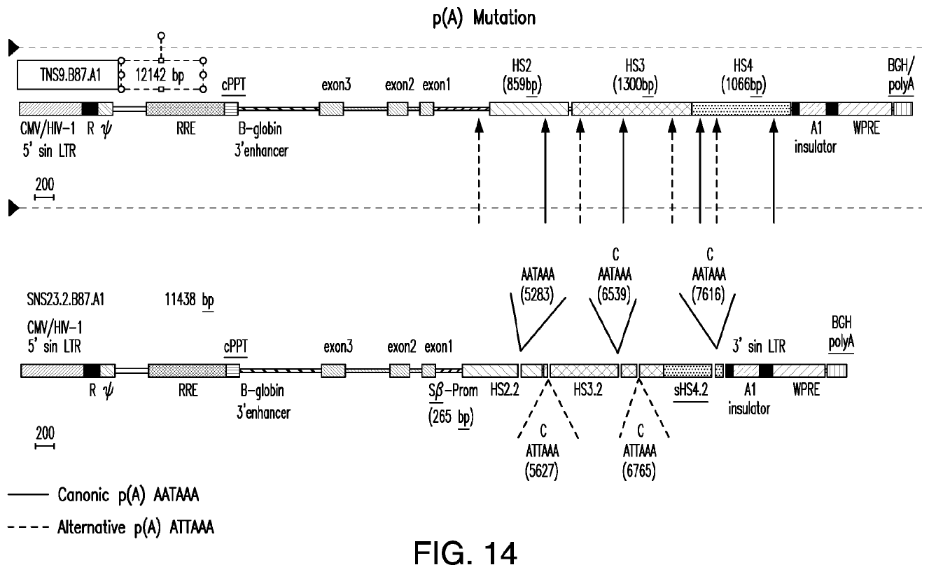

In certain non-limiting embodiments, the β-globin LCR comprises or consists essentially of a HS2 region having the nucleotide sequence set forth in SEQ ID NO: 33; a HS3 region having the nucleotide sequence set forth in SEQ ID NO: 34; and a HS4 region having the nucleotide sequence set forth in SEQ ID NO: 35, and the β-globin LCR does not comprise a HS1 region, e.g., the β-globin LCR of SNS23.2.B87.A1 shown in FIGS. 13 and 14.

In certain non-limiting embodiments, the β-globin LCR further comprises a HS1 region, i.e., a β-globin LCR comprising a HS1 region, a HS2 region, a HS3 region, and a HS4 region. In certain embodiments, the HS1 region, HS2 region, HS3 region and HS4 region within the β-globin LCR are contiguous. In certain non-limiting embodiments, the β-globin LCR consisting essentially of a HS1 region, a HS2 region, a HS3 region and a HS4 region. In certain embodiments, the β-globin LCR comprises two introduced GATA-1 binding sites at the junction between the HS3 region and the HS4 region.

The length and the sequence of the HS1 region can vary. In certain embodiments, the HS1 region is from about 300 bp to about 1500 bp in length, e.g., from about 300 bp to about 1100 bp in length. In certain embodiments, the HS1 region has a length of about 1.0 kb or more, e.g., about 1.1 kb, about 1.2 kb, about 1.3 kb, about 1.4 kb, or about 1.5 kb. In certain embodiments, the HS1 region has a length of about 1.1 kb. In certain non-limiting embodiments, the HS1 region has a length of 1074 bp. In certain non-limiting embodiments, the HS1 region comprises or has the nucleotide sequence set forth in SEQ ID NO:2, which is provided below:

[SEQ ID NO: 2]
AAGTAAACTTCCACAACCGCAAGCTTATTGAGGCTAAGGCATCTGTG
AAGGAAAGAAACATCTCCTCTAAACCACTATGCTGCTAGAGCCTCTT
TTCTGTACTCAAGCCTCATTCAGACACTAGTGTCACCAGTCTCCTCA
TATACCTATTGTATTTTCTTCTTCTTGCTGGTTTAGTCATGTTTTCT
GGGAGCTTAGGGGCTTATTTTATTTTGTTTTGTTTTCTAATCAACAG
AGATGGGCAAACCCATTATTTTTTTCTTTAGACTTGGGATGGTGATA
GCTGGGCAGCGTCAGAAACTGTGTGTGGATATAGATAAGAGCTCGGA
CTATGCTGAGCTGTGATGAGGGAGGGACCTAGCCAAAGGCAGTGAGA
GTCAGAATGCTCCTGCTATTGCCTTCTCAGTCCCCACGCTTGGTTTC
TACACAAGTAGATACATAGAAAAGGCTATAGGTTAGTGTTTGAGAGT
CCTGCATGAGTTAGTTGCTCAGAAATGCCCGATAAATATGTTATGTG
TGTTTATGTATATATATGTTTTATATATATATATGTGTGTGTGTGTG
TGTGTGTGTGTTGTGTTTACAAATATGTGATTATCATCAAAACGTGA
GGGCTAAAGTGACCAGATAACTTGCAGGTCCTAGGATACCAGGAAAA
TAAATTACATTCCAAAAATTTAACTGAGACTTTAAAAAAAAAAAAAA
AAAAAAAAAAAAAACCAGTGATCCATGGACACAGGGAGGGGAACATC
ACACACTGGGGCCTGTTGGGGGTGGGGGGCTAGGGGAAGGATAGCAT
TAGGAGAAATACCTAATGTAGATGACGGGTTGATGGGTGCAGCAAAC
CACCATGGCACATGTACCCCAGAACTTAAAGCATATTAAAAAAACAG
TGATCATAAAAGAAGCTCAAATTTAACTATAAGAGACGGAATGGCTC
CCACAATTCTTAACTATAATCTTACAGAATATTCTCATTGAATAGAA
GTATGCTTATCATTAGAGATTTGGACAGCCAGGAAAGCACAGAAAAA
AAAAAAAGGAGCTCTGTTGCCTTATAGCCTAGAGGTGTTT

In certain embodiments, the HS1 region has a length of less than about 1.0 kb, e.g., from about 400 bp to about 700 bp, from about 400 bp to about 500 bp, from about 500 bp to about 600 bp, from about 600 bp to about 700 bp, from about 700 bp to about 800 bp, from about 800 bp to about 900 bp, or from about 900 bp to about 1.0 kb. In certain embodiments, the HS1 region has a length of less than about 700 bp. In certain embodiments, the HS1 region has a length of about 600 bp. In certain non-limiting embodiments, the HS1 region has a length of 602 bp. In certain non-limiting embodiments, the HS1 region comprises or has the nucleotide sequence set forth in SEQ ID NO:3, which is provided below:

[SEQ ID NO: 3]
GGCATCTGTGAAGGAAAGAAACATCTCCTCTAAACCACTATGCTGCT
AGAGCCTCTTTTCTGTACTCAAGCCTCATTCAGACACTAGTGTCACC
AGTCTCCTCATATACCTATTGTATTTTCTTCTTCTTGCTGGTTTAGT
CATGTTTTCTGGGAGCTTAGGGGCTTATTTTATTTTGTTTTGTTTTC
TAATCAACAGAGATGGGCAAACCCATTATTTTTTTCTTTAGACTTGG
GATGGTGATAGCTGGGCAGCGTCAGAAACTGTGTGTGGATATAGATA
AGAGCTCGGACTATGCTGAGCTGTGATGAGGGAGGGACCTAGCCAAA
GGCAGTGAGAGTCAGAATGCTCCTGCTATTGCCTTCTCAGTCCCCAC
GCTTGGTTTCTACACAAGTAGATACATAGAAAAGGCTATAGGTTAGT
GTTTGAGAGTCCTGCATGAGTTAGTTGCTCAGAAATGCCCGATAAAT
ATGTTATGTGTGTTTATGTATATATATGTTTTATATATATATATGTG
TGTGTGTGTGTGTGTGTGTGTTGTGTTTACAAATATGTGATTATCAT
CAAAACGTGAGGGCTAAAGTGACCAGATAACTTGCAGG

In certain embodiments, the HS1 region has a length of less than about 500 bp. In certain embodiments, the HS1 region has a length of about 490 bp. In certain non-limiting embodiments, the HS1 region has a length of 489 bp. In certain non-limiting embodiments, the HS1 region comprises or has the nucleotide sequence set forth in SEQ ID NO:4, which is provided below:

[SEQ ID NO: 4]
GGCATCTGTGAAGGAAAGAAACATCTCCTCTAAACCACTATGCTGCT
AGAGCCTCTTTTCTGTACTCAAGCCTCATTCAGACACTAGTGTCACC
AGTCTCCTCATATACCTATTGTATTTTCTTCTTCTTGCTGGTTTAGT
CATGTTTTCTGGGAGCTTAGGGGCTTATTTTATTTTGTTTTGTTTTC
TAATCAACAGAGATGGGCAAACCCATTATTTTTTTCTTTAGACTTGG
GATGGTGATAGCTGGGCAGCGTCAGAAACTGTGTGTGGATATAGATA
AGAGCTCGGACTATGCTGAGCTGTGATGAGGGAGGGACCTAGCCAAA
GGCAGTGAGAGTCAGAATGCTCCTGCTATTGCCTTCTCAGTCCCCAC
GCTTGGTTTCTACACAAGTAGATACATAGAAAAGGCTATAGGTTAGT
GTTTGAGAGTCCTGCATGAGTTAGTTGCTCAGAAATGCCCGATAAAT
ATGTTATGTGTGTTTATGT

In certain non-limiting embodiments, the β-globin LCR comprises or consists essentially of a HS1 region disclosed herein, a HS2 region disclosed herein, a HS3 region disclosed herein, and a HS4 region disclosed herein.

In certain non-limiting embodiments, the β-globin LCR comprises or consists essentially of a HS1 region having the nucleotide sequence set forth in SEQ ID NO: 3, SEQ ID NO: 4, SEQ ID NO: 22 or SEQ ID NO: 23; a HS2 region having the nucleotide sequence set forth in SEQ ID NO: 9 or a consecutive portion thereof, a modification of SEQ ID NO: 9, a modification of a consecutive portion of SEQ ID NO: 9 (e.g., SEQ ID NO: 33), SEQ ID NO:20, SEQ ID NO:21; a HS3 region having the nucleotide sequence set forth in SEQ ID NO: 5 or a modification thereof (e.g., SEQ ID NO: 34); and a HS4 region having the nucleotide sequence set forth in SEQ ID NO: 6 or a consecutive portion thereof, SEQ ID NO: 7 or a consecutive portion thereof, a modification of SEQ ID NO: 6, a modification of a consecutive portion of SEQ ID NO: 6 (e.g., SEQ ID NO: 35), a modification of SEQ ID NO: 7, or a modification of a consecutive portion of SEQ ID NO: 7, or SEQ ID NO:8.

In certain non-limiting embodiments, the β-globin LCR comprises or consists essentially of a HS1 region having the nucleotide sequence set forth in SEQ ID NO: 3; a HS2 region having nucleotides 45 to 860 of SEQ ID NO: 9; a HS3 region having the nucleotide sequence set forth in SEQ ID NO: 5; and a HS4 region having nucleotides 115 to 868 of SEQ ID NO: 6 e.g., the β-globin LCR of SNS22.B87.A1 shown in FIG. 15.

In certain non-limiting embodiments, the β-globin LCR comprises or consists essentially of a HS1 region having the nucleotide sequence set forth in SEQ ID NO: 3; a HS2 region having nucleotide sequence set forth in SEQ ID NO: 33; a HS3 region having the nucleotide sequence set forth in SEQ ID NO: 34; and a HS4 region having the nucleotide sequence set forth in SEQ ID NO: 35, e.g., as the β-globin LCR of SNS22.2.B87.A1 shown in FIG. 13.

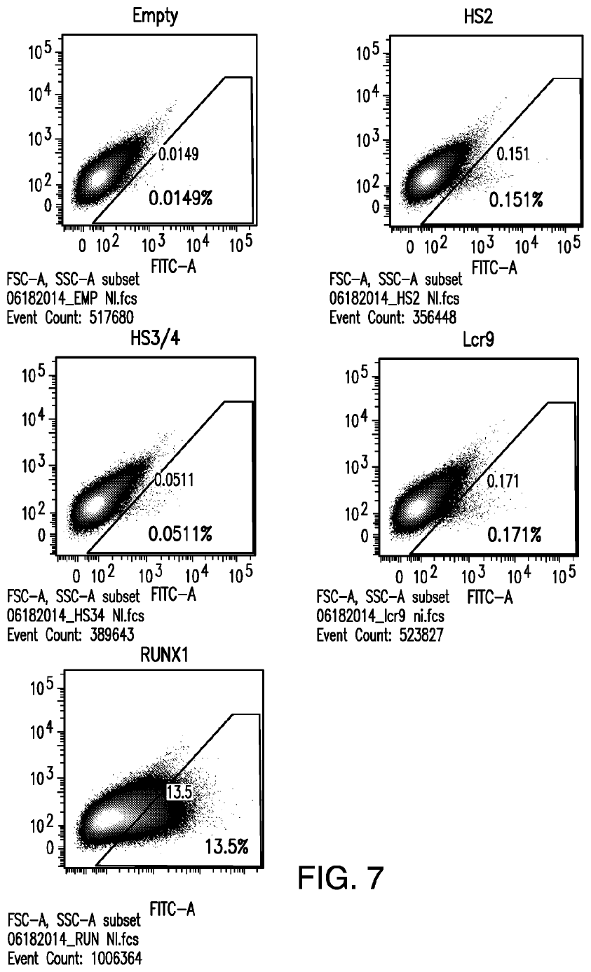

Recent studies have shown that HS2 is not erythroid-specific, but is expressed in other cell lines and lineages (See Example 3 and FIG. 7) and is also present in undifferentiated human embryonic stem cells (Chang et al., Stem cell reviews (2013); 9:397-407). Due to the non-erythroid activity of HS2, HS2-containing globin vectors may pose a risk for their safe use in clinical treatment, e.g., for treating thalassemia and sickle cell patients. In certain embodiments, the β-globin LCR does not comprise a HS2 region. In certain embodiments, the β-globin LCR does not comprise a core sequence of HS2. A core sequence of HS2 provides position independent, high level expression. In addition, a core sequence of HS2 sustains the enhancer activity of HS2. For example, the core sequence of HS2 enhances the transcription of a globin gene (e.g., human β-globin gene). Additionally, a core sequence of HS2 comprises one or more binding sites or binding motifs for ubiquitous as well as tissue-specific (e.g., erythroid-specific) proteins (e.g., transcription factors), including, but not limited to, members of AP1 family of proteins (e.g., NF-E2), GATA-1 (also known as “NF-E1” or “NFE1”), Krüppel-like Zn finger proteins (e.g., ubiquitous proteins Sp1 and YY1, and erythroid-restricted factor erythroid Krüppel-like factor (EKLF)), and basic helix-loop-helix (bHLH) proteins (E boxes) (e.g., USF and TAL1). AP1 binding sites are required for enhancement and induction (Moi and Kan (1990); Ney et al., (1990); Talbot and Grosveld (1991)). Furthermore, binding of NF-E2 can cause disruption of in vitro reconstituted chromatin at HS2 (Armstrong and Emerson (1996)). Mutations in the GATA-1 binding sites can cause a reduction in enhancer activity of HS2 in transgenic mice (Caterina et al., (1994)). Although both AP1 (e.g., AP1/NF-E2) and GATA1 binding sites are important for core function, mice lacking these factors do not show impaired globin gene expression (Weiss et al., 1994).

In certain embodiments, the β-globin LCR does not comprise the full length of a core sequence of HS2. In certain embodiments, the core sequence of a HS2 region is a core sequence of human HS2. In certain non-limiting embodiments, the core sequence of human HS2 comprises a tandem pair of binding sites for members of AP1 family of proteins (e.g., NF-E2) (referred to as “AP1/NF-E2” binding sites) (e.g., GCTGAGTCA, and GATGAGTCA), one binding site for Kruppel-like Zn finger proteins (e.g., AGGGTGTGT), one GATA-1 binding site (e.g., CTATCT), and three E boxes (CANNTG, e.g., CAGATG, and CACCTG). In certain non-limiting embodiments, the β-globin LCR does not comprise the full length of a 388 bp core sequence of human HS2, which has the nucleotide sequence set forth in SEQ ID NO:20 provided below:

[SEQ ID NO: 20]
TAAGCTTCAGTTTTTCCTTAGTTCCTGTTACATTTCTGTGTGTCTCC
ATTAGTGACCTCCCATAGTCCAAGCATGAGCAGTTCTGGCCAGGCCC
CTGTCGGGGTCAGTGCCCCACCCCCGCCTTCTGGTTCTGTGTAACCT
TCTAAGCAAACCTTCTGGCTCAAGCACAGCAATGCTGAGTCATGATG
AGTCATGCTGAGGCTTAGGGTGTGTGCCCAGATGTTCTCAGCCTAGA
GTGATGACTCCTATCTGGGTCCCCAGCAGGATGCTTACAGGGCAGAT
GGCAAAAAAAAGGAGAAGCTGACCACCTGACTAAAACTCCACCTCAA
ACGGCATCATAAAGAAAATGGATGCCTGAGACAGAATGTGACATATT
CTAGAATATATT

The nucleotide sequence set forth in SEQ ID NO:20 corresponds to nucleotides position 16671 to position 17058 of SEQ ID NO: 19 (GenBank Access No.: NG_000007.3). In SEQ ID NO:20, one AP1/NF-E2 binding site having the nucleotide sequence of GCTGAGTCA is located at position 175 to position 183, one AP1/NF-E2 binding site having the nucleotide sequence of GATGAGTCA is located at position 185 to position 193, one binding site for Krüppel-like Zn finger proteins having the nucleotide sequence of AGGGTGTGT is located as position 205 to position 213, two E boxes, each of which have the nucleotide sequence of CAGATG, is located at position 217 to position 222, and position 278 to position 283, one GATA-1 binding site having the nucleotide sequence of CTATCT is located at position 246 to position 251, one E box having the nucleotide sequence of CACCTG is located at position 306 to position 311.

In certain non-limiting embodiments, the β-globin LCR does not comprise the full length of a 387 bp core sequence of human HS2, which has the nucleotide sequence set forth in SEQ ID NO:21 provided below:

[SEQ ID NO: 21]
TAAGCTTCAGTTTTTCCTTAGTTCCTGTTACATTTCTGTGTGTCTCC
ATTAGTGACCTCCCATAGTCCAAGCATGAGCAGTTCTGGCCAGGCCC
CTGTCGGGGTCAGTGCCCCACCCCCGCCTTCTGGTTCTGTGTAACCT
TCTAAGCAAACCTTCTGGCTCAAGCACAGCAATGCTGAGTCATGATG
AGTCATGCTGAGGCTAGGGTGTGTGCCCAGATGTTCTCAGCCTAGAG
TGATGACTCCTATCTGGGTCCCCAGCAGGATGCTTACAGGGCAGATG
GCAAAAAAAAGGAGAAGCTGACCACCTGACTAAAACTCCACCTCAAA
CGGCATCATAAAGAAAATGGATGCCTGAGACAGAATGTGACATATTC
TAGAATATATT

In SEQ ID NO:21, one AP1/NF-E2 binding site having the nucleotide sequence of GCTGAGTCA is located at position 175 to position 183, one AP1/NF-E2 binding site having the nucleotide sequence of GATGAGTCA is located at position 185 to position 193, one binding site for Krüppel-like Zn finger proteins having the nucleotide sequence of AGGGTGTGT is located as position 204 to position 212, two E boxes, each of which have the nucleotide sequence of CAGATG, is located at position 216 to position 221, and position 277 to position 282, one GATA-1 binding site having the nucleotide sequence of CTATCT is located at position 245 to position 250, one E box having the nucleotide sequence of CACCTG is located at position 305 to position 310.

In certain embodiments, the β-globin LCR does not comprise a HS2 region that comprises a core sequence of HS2. A HS2 region that comprises a core sequence of HS2 can vary in length and sequence. In non-limiting examples, a HS2 region that comprises a core sequence of HS2 is from about 400 bp to about 1000 bp, e.g., from about 400 bp to about 500 bp, from about 500 bp to about 600 bp, from about 600 bp to about 700 bp, from about 700 bp to about 800 bp, from about 800 bp to about 900 bp, or from about 900 bp to about 1000 bp, in length. In certain non-limiting embodiments, the β-globin LCR does not comprise a 840 bp HS2 region (e.g., the HS2 region comprised in the globin vector TNS9 disclosed in U.S. Pat. No. 7,541,179). In certain non-limiting embodiments, the β-globin LCR does not comprise a 860 bp HS2 region. In certain non-limiting embodiments, the β-globin LCR does not comprise an about 650 bp HS2 region. In certain non-limiting embodiments, the β-globin LCR does not comprise a 646 bp HS2 region (e.g., the HS2 region comprised in the globin vector LentiGlobin™, also known as “B87”). In certain non-limiting embodiments, the β-globin LCR does not comprise an about 420 bp HS2 region. In certain non-limiting embodiments, the β-globin LCR does not comprise a 423 bp HS2 region (e.g., the HS2 region comprised in the globin vector disclosed in Sadelain et al., Proc. Nat'l Acad. Sci. (USA) (1995); 92:6728-6732).

In certain embodiments, the β-globin LCR does not comprise a HS2 region that sustains the enhancer activity of HS2. In certain embodiments, the β-globin LCR does not comprise a HS2 region that is capable of enhancing the transcription of a globin gene (e.g., human β-globin gene). In non-limiting examples, the β-globin LCR does not comprise a HS2 region whose ability to enhance the transcription of a globin gene (e.g., human β-globin gene) is no less than about 60%, no less than about 70%, no less than about 80%, no less than about 90%, or no less than about 95% in comparison to a native HS2 region.

In certain embodiments, the β-globin LCR does not comprise a HS2 region that comprises one, two, three, four, five, six or seven of the following binding sites: two (a tandem pair of) AP1/NF-E2 binding sites (e.g., GCTGAGTCA, and GATGAGTCA), one binding site for Kruppel-like Zn finger proteins (e.g., AGGGTGTGT), one GATA-1 binding site (e.g., CTATCT), and three E boxes (CANNTG, e.g., CAGATG, and CACCTG). In certain embodiments, the β-globin LCR does not comprise a HS2 region that comprises six of the above-described binding sites. For example, in certain embodiments, the β-globin LCR does not comprise a HS2 region that comprises two AP1/NF-E2 binding sites, one binding site for Kruppel-like Zn finger proteins, one GATA-1 binding site, and two not three E boxes. In certain embodiments, the β-globin LCR does not comprise a HS2 region that comprises one not two AP1/NF-E2 binding site, one binding site for Kruppel-like Zn finger proteins, one GATA-1 binding site, and three E boxes. In certain embodiments, the β-globin LCR does not comprise a HS2 region that comprises two AP1/NF-E2 binding sites, one GATA-1 binding site, and three E boxes and does not comprise one binding site for Kruppel-like Zn finger proteins. In certain embodiments, the β-globin LCR does not comprise a HS2 region that comprises two AP1/NF-E2 binding sites, one binding site for Kruppel-like Zn finger proteins, and three E boxes, and does not comprise one GATA-1 binding site.

In certain embodiments, the β-globin LCR comprises a HS1 region, a HS3 region, and a HS4 region, and does not comprise a HS2 region. In certain embodiments, the HS1 region, HS3 region and HS4 region within the β-globin LCR are contiguous. In certain non-limiting embodiments, the β-globin LCR consists essentially of a HS1 region, a HS3 region and a HS4 region. In certain embodiments, the β-globin LCR comprises two introduced GATA-1 binding sites at the junction between the HS3 region and the HS4 region. The HS3 region can lie between the HS1 region and the HS4 region. The HS1, HS3, and HS4 regions can be any HS1, HS3, and HS4 regions disclosed herein. The HS2 region can also be any HS2 region disclosed herein.

In certain non-limiting embodiments, the β-globin LCR comprises or consists essentially of a HS1 region disclosed herein, a HS3 region disclosed herein, and a HS4 region disclosed herein, and does not comprise a HS2 region.

In certain non-limiting embodiments, the β-globin LCR comprises or consists essentially of a HS1 region having the nucleotide sequence set forth in SEQ ID NO:2, SEQ ID NO:3, SEQ ID NO:4, SEQ ID NO:22 or SEQ ID NO:23; a HS3 region having the nucleotide sequence set forth in SEQ ID NO:5 or a modification thereof (e.g., SEQ ID NO: 34); and a HS4 region having the nucleotide sequence set forth in SEQ ID NO:6 or a consecutive portion thereof, SEQ ID NO:7 or a consecutive portion thereof, a modification of SEQ ID NO: 6, a modification of a consecutive portion of SEQ ID NO: 6 (e.g., SEQ ID NO: 35), a modification of SEQ ID NO: 7, or a modification of a consecutive portion of SEQ ID NO: 7, or SEQ ID NO:8, and the β-globin LCR does not comprise a HS2 region.

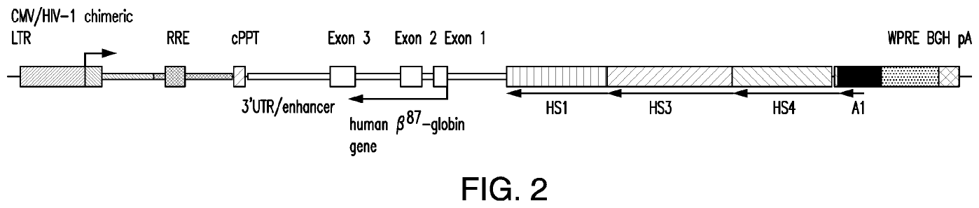

In certain non-limiting embodiments, the β-globin LCR comprises or consists essentially of a HS1 region having the nucleotide sequence set forth in SEQ ID NO:2, a HS3 region having the nucleotide sequence set forth in SEQ ID NO:5, and a HS4 region having the nucleotide sequence set forth in SEQ ID NO:6, and the β-globin LCR does not comprise a HS2 region, as shown in FIG. 2.

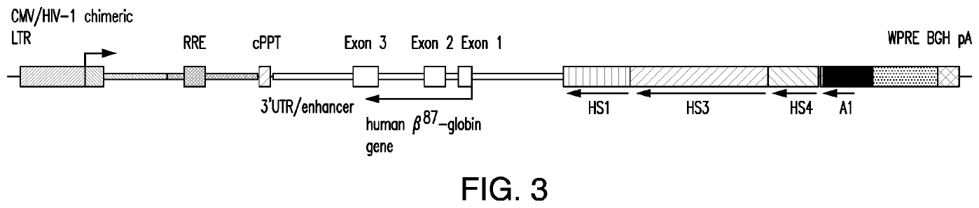

In certain non-limiting embodiments, the β-globin LCR comprises or consists essentially of a HS1 region having the nucleotide sequence set forth in SEQ ID NO:3, a HS3 region having the nucleotide sequence set forth in SEQ ID NO:5, and a HS4 region having the nucleotide sequence set forth in SEQ ID NO:8, and the β-globin LCR does not comprise a HS2 region, as shown in FIG. 3.

In certain non-limiting embodiments, the β-globin LCR comprises or consists essentially of a HS1 region having the nucleotide sequence set forth in SEQ ID NO:4, a HS3 region having the nucleotide sequence set forth in SEQ ID NO:5, and a HS4 region having the nucleotide sequence set forth in SEQ ID NO:8, and the β-globin LCR does not comprise a HS2 region.

In certain non-limiting embodiments, the β-globin LCR comprises or consists essentially of a HS1 region having the nucleotide sequence set forth in SEQ ID NO: 3; a HS3 region having the nucleotide sequence set forth in SEQ ID NO: 5; and a HS4 region having nucleotides 115 to 868 of SEQ ID NO: 6 e.g., the β-globin LCR of SNS24.B87.A1 shown in FIG. 15.

In certain non-limiting embodiments, the β-globin LCR comprises or consists essentially of a HS1 region having the nucleotide sequence set forth in SEQ ID NO: 3; a HS3 region having the nucleotide sequence set forth in SEQ ID NO: 34; and a HS4 region having the nucleotide sequence set forth in SEQ ID NO: 35, e.g., as the β-globin LCR of SNS24.2.B87.A1 shown in FIG. 13.

In certain embodiments, the β-globin LCR does not comprise a HS1 region or a HS2 region. In certain embodiments, the β-globin LCR does not comprise a core sequence of HS1. A core sequence of HS1 sustains the activity of HS1, e.g., enhancer activity, or functioning as a facilitator or regulatory element to tether the enhancer activity of other HS regions, e.g., HS2-4. In addition, a core sequence of HS1 comprises one or more binding sites or binding motifs for ubiquitous as well as tissue-specific (e.g., erythroid-specific) proteins (e.g., transcription factors), including, but not limited to, GATA-1, and Krüppel-like Zn finger proteins (e.g., erythroid-restricted factor EKLF).

In certain embodiments, the β-globin LCR does not comprise the full length of a core sequence of HS1. In certain embodiments, the core sequence of a HS1 region is a core sequence of human HS1. In certain non-limiting embodiments, the core sequence of human HS1 comprises two GATA-1 binding sites (e.g., TTATCT, and CTATCA), and one binding site for EKLF (e.g., CCACACACA). In certain embodiments, the β-globin LCR does not comprise the full length of a 286 bp core sequence of human HS1. In certain non-limiting embodiments, the 286 bp core sequence of human HS1 has the nucleotide sequence set forth in SEO ID NO:22 provided below:

[SEQ ID NO: 22]
CTGAGCAACTAACTCATGCAGGACTCTCAAACACTAACCTATAGCCT
TTTCTATGTATCTACTTGTGTAGAAACCAAGCGTGGGGACTGAGAAG
GCAATAGCAGGAGCATTCTGACTCTCACTGCCTTTGGCTAGGTCCCT
CCCTCATCACAGCTCAGCATAGTCCGAGCTCTTATCTATATCCACAC
ACAGTTTCTGACGCTGCCCAGCTATCACCATCCCAAGTCTAAAGAAA
AAAATAATGGGTTTGCCCATCTCTGTTGATTAGAAAACAAAACAAAA
TAAA

In SEQ ID NO:22, one GATA-1 binding site having the nucleotide sequence of TTATCT is located at position 173 to position 178, one GATA-1 binding site having the nucleotide sequence of CTATCA located at position 210 to position 215, and one binding site for EKLF having the nucleotide sequence of CCACACACA is located at position 183 to position 191.

In certain non-limiting embodiments, the 286 bp core sequence of human HS1 has the nucleotide sequence set forth in SEQ ID NO:23 provided below:

[SEQ ID NO: 23]
CTGAGCAACTAATCATGCAGGACTCTCAAACACTAACCTATAGCCTT
TTCTATGTATCTACTTGTGTAGAAACCAAGCGTGGGGACTGAGAAGG
CAATAGCAGGAGCATTCTGACTCTCACTGCCTTTAGCTAGGCCCCTC
CCTCATCACAGCTCAGCATAGTCCTGAGCTCTTATCTATATCCACAC
ACAGTTTCTGACGCTGCCCAGCTATCACCATCCCAAGTCTAAAGAAA
AAAATAATGGGTTTGCCCATCTCTGTTGATTAGAAAACAAAACAAAA
TAAA

The nucleotide sequence set forth in SEQ ID NO:23 corresponds to nucleotides position 21481 to position 21766 of SEQ ID NO:19 (GenBank Access No.: NG_000007.3). In SEQ ID NO:23, one GATA-1 binding site having the nucleotide sequence of TTATCT is located at position 173 to position 178, one GATA-1 binding site having the nucleotide sequence of CTATCA located at position 210 to position 215, and one binding site for EKLF having the nucleotide sequence of CCACACACA is located at position 183 to position 191.

In certain embodiments, the β-globin LCR does not comprise a HS1 region that comprises a core sequence of HS1. A HS1 region that comprises a core sequence of HS1 can vary in length and sequence. In non-limiting examples, a HS1 region that comprises a core sequence of HS1 is from about 300 bp to about 1200 bp, e.g., from about 300 bp to about 400 bp, from about 400 bp to about 500 bp, from about 500 bp to about 600 bp, from about 600 bp to about 700 bp, from about 700 bp to about 800 bp, from about 800 bp to about 900 bp, from about 900 bp to about 1000 bp, from about 1000 bp to about 1100 bp, or from about 1100 bp to about 1200 bp, in length. In certain non-limiting embodiments, the β-globin LCR does not comprise an about 1.0 kb bp HS1 region. In certain non-limiting embodiments, the β-globin LCR does not comprise an about 1.1 kb HS1 region.

In certain embodiments, the β-globin LCR does not comprise a HS1 region that sustains the activity of HS1, e.g., enhancer activity, or functioning as a facilitator or regulatory element to tether the enhancer activity of other HS regions, e.g., HS2-4. In certain embodiments, the β-globin LCR does not comprise a HS1 region that is capable of enhancing the transcription of a globin gene (e.g., human β-globin gene). In non-limiting examples, the β-globin LCR does not comprise a HS1 region whose ability to enhance the transcription of a globin gene (e.g., human β-globin gene) is no less than about 60%, no less than about 70%, no less than about 80%, no less than about 90%, or no less than about 95% in comparison to a native HS1 region. In non-limiting examples, the β-globin LCR does not comprise a HS1 region whose ability to tether the enhancer activity of one or more of HS2-HS4 is no less than about 60%, no less than about 70%, no less than about 80%, no less than about 90%, or no less than about 95% in comparison to a native HS1 region.

In certain embodiments, the β-globin LCR does not comprise a HS1 region that comprises one, two, or three of the following binding sites: two GATA-1 binding sites (e.g., TTATCT, and CTATCA), and one binding site for EKLF (e.g., CCACACACA). In certain embodiments, the β-globin LCR does not comprise a HS1 region that comprises two of the above-described binding sites. For example, in certain embodiments, the β-globin LCR does not comprise a HS1 region that comprises two GATA-1 binding sites and does not comprise one binding site for EKLF. In certain embodiments, the β-globin LCR does not comprise a HS1 region that comprises one not two AP1/NF-E2 binding site and one binding site for EKLF.

In certain embodiments, the β-globin LCR comprises a HS3 region and a HS4 region, and the β-globin LCR does not comprise a HS1 region or a HS2 region. In certain embodiments, the HS3 region and HS4 region within the β-globin LCR are contiguous. In certain non-limiting embodiments, the β-globin LCR consisting essentially of a HS3 region and a HS4 region. In another embodiment, the β-globin LCR comprises two introduced GATA-1 binding sites at the junction between the HS3 region and the HS4 region. The HS3 region can lie between the globin gene or functional portion thereof and the HS4 region.

In certain embodiments, the β-globin LCR comprises a HS3 region having the nucleotide sequence set forth in SEQ ID NO:5 and a HS4 region having the nucleotide sequence set forth in SEQ ID NO:6, SEQ ID NO: 7 or SEQ ID NO:8, and the β-globin LCR does not comprise a HS1 region or a HS2 region.

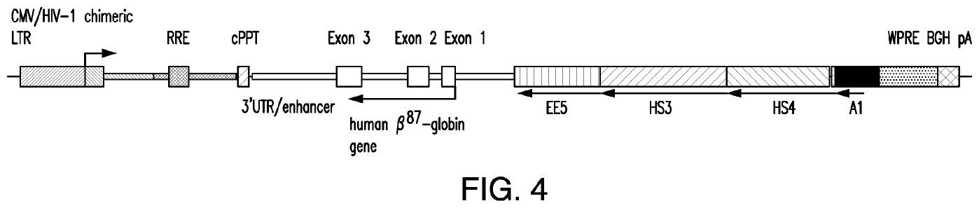

In certain non-limiting embodiments, the β-globin LCR comprises a HS3 region having the nucleotide sequence set forth in SEQ ID NO:5 and a HS4 region having the nucleotide sequence set forth in SEQ ID NO:6, and the β-globin LCR does not comprise a HS1 region or a HS2 region, as shown in FIG. 4.

In certain embodiments, the β-globin LCR does not comprise a HS1 region, a HS2 region, or a HS4 region. In certain embodiments, the β-globin LCR does not comprise the full length of a core sequence of HS4. In certain embodiments, the core sequence of a HS4 region is a core sequence of human HS4. In certain embodiments, the β-globin LCR does not comprise the full length of a 280 bp core sequence of human HS4 (e.g., the 280 bp core sequence of human HS4 disclosed in Pruzina et al., Nucleic Acdis Research (1991); 19:7:1413-1419).

In certain embodiments, the β-globin LCR does not comprise a HS4 region that comprises a core sequence of HS4. A HS4 region that comprises a core sequence of HS4 can vary in length and sequence. In non-limiting examples, a HS4 region that comprises a core sequence of HS4 is from about 300 bp to about 1200 bp, e.g., from about 300 bp to about 400 bp, from about 400 bp to about 500 bp, from about 500 bp to about 600 bp, from about 600 bp to about 700 bp, from about 700 bp to about 760 bp, from about 700 bp to about 800 bp, from about 800 bp to about 900 bp, from about 900 bp to about 1000 bp, from about 1000 bp to about 1100 bp, or from about 1100 bp to about 1200 bp, in length. In certain non-limiting embodiments, the β-globin LCR does not comprise an about 1.0 kb HS4 region. In certain non-limiting embodiments, the β-globin LCR does not comprise an about 1.1 kb HS4 region. In certain non-limiting embodiments, the β-globin LCR does not comprise an about 760 bp HS4 region. In certain non-limiting embodiments, the β-globin LCR does not comprise an about 754 bp HS4 region.

In certain embodiments, the β-globin LCR does not comprise a HS4 region that sustains the activity of HS4, e.g., enhancer activity. In certain embodiments, the β-globin LCR does not comprise a HS4 region that is capable of enhancing the transcription of a globin gene (e.g., human β-globin gene), conferring position independent expression, and/or increasing β-globin transgene expression. In certain embodiments, the expression cassette comprises at least one erythroid-specific enhancer, which can compensate the activities of HS1, HS2, and/or HS4 regions.

In certain embodiments, the β-globin LCR comprises a HS3 region, and the β-globin LCR does not comprise a HS1 region, a HS2 region or a HS4 region. In certain embodiments, the β-globin LCR consisting essentially of a HS3 region. In certain embodiments, the β-globin LCR comprises a HS3 region having the nucleotide sequence set forth in SEQ ID NO: 5 and a HS4 region having the nucleotide sequence set forth in SEQ ID NO:6, SEQ ID NO:7 or SEQ ID NO:8, and the β-globin LCR does not comprise a HS1 region or a HS2 region. In certain embodiments, the expression cassette comprises at least one erythroid-specific enhancer.

In certain non-limiting embodiments, the β-globin LCR comprises a HS3 region having the nucleotide sequence set forth in SEQ ID NO:5, and the β-globin LCR does not comprise a HS1 region, a HS2 region or a HS4 region. In certain embodiments, the expression cassette comprises at least one erythroid-specific enhancer.

In certain non-limiting embodiments, the β-globin LCR comprises a HS3 region having the nucleotide sequence set forth in SEQ ID NO: 34, and the β-globin LCR does not comprise a HS1 region, a HS2 region or a HS4 region, e.g., the β-globin LCR of SNS27.2.B87.A1 shown in FIG. 13. In certain embodiments, the expression cassette comprises at least one erythroid-specific enhancer, e.g., SNS27.2.B87.A1 shown in FIG. 13.

### 3.2. Globin Gene

In accordance with the presently disclosed subject matter, the expression cassette comprises a globin gene or a functional portion thereof. The globin gene can be a β-globin gene, a γ-globin gene, or a δ-globin gene. In certain embodiments, the expression cassette comprises a human β-globin gene. In accordance with the presently disclosed subject matter, the human β-globin gene can be a wild-type human β-globin gene, a deleted human β-globin gene comprising one or more deletions of intron sequences, or a mutated human β-globin gene encoding at least one anti-sickling amino acid residue. In certain non-limiting embodiments, a presently disclosed expression cassette comprises a wild-type human β-globin gene. A wild-type human β-globin gene comprises three exons (exon 1, exon 2, and exon 3). In certain embodiments, a presently disclosed expression cassette comprises a non-wild-type (mutated or modified) human β^{A}-globin gene. In certain embodiments, a presently disclosed expression cassette comprises a human β-globin gene with a deletion in intron 2 (IVS2). In certain embodiments, the deletion in IVS2 is about 370 bp. The deletion in IVS2 can eliminate AT-rich (ATR) sequences that comprise a cryptic polyadenylation site responsible of premature termination of the transcription. In certain embodiments, a presently disclosed expression cassette comprises a human β^{A}-globin gene encoding a threonine to glutamine mutation at codon 87 (β^{A-T87Q}). The glutamine residue at position 87 in the gamma-globin chain augments the anti-sickling activity of the gamma chain relative to the beta chain, while preserving adult oxygen-binding characteristics of the beta chain (Nagel et al., Proc. Natl. Acad. Sci. U.S.A. (1979); 76:670-672). In certain embodiments, a functional portion of a globin gene has at least about 80%, at least about 90%, at least about 95%, at least about 99% or at least about 100% identity to a corresponding wild-type reference polynucleotide sequence.

In certain embodiments, the human β^{A}-globin gene is a human β^{A}-globin gene encoding a threonine to glutamine mutation at codon 87 (β^{A-T87Q}). In certain embodiments, the human β^{A}-globin gene encoding a threonine to glutamine mutation at codon 87 (β^{A-T87Q}) further comprises a deletion in intron 2 (e.g., an about 370 bp deletion). In certain embodiments, the human β^{A}-globin gene encoding a threonine to glutamine mutation at codon 87 (β^{A-T87Q}) comprises the nucleotide sequence set forth in SEQ ID NO: 36, which is provided below.

[SEQ ID NO: 36]
gc aatgaaaata aatgtttttt attaggcaga atccagatgc
tcaaggccct tcataatatc ccccagttta gtagttggac
ttagggaaca aaggaacctt taatagaaat tggacagcaa
gaaagcgagc ttagtgatac ttgtgggcca gggcattagc
cacaccagcc accactttct gataggcagc ctgcactggt
ggggtgAATT CTTTGCCAAA GTGATGGGCC AGCACACAGA
CCAGCACGTT GCCCAGGAGC TGTGGGAGGA AGATAAGAGG
TATGAACATG ATTAGCAAAA GGGCCTAGCT TGGACTCAGA
ATAATCCAGC CTTATCCCAA CCATAAAATA AAAGCAGAAT
GGTAGCTGGA TTGTAGCTGC TATTAGCAAT ATGAAACCTC
TTACATCAGT TACAATTTAT ATGCAGAAAT ATTTATATGC
AGAAATATTG CTATTGCCTT AACCCAGAAA TTATCACTGT
TATTCTTTAG AATGGTGCAA AGAGGCATGA TACATTGTAT
CATTATTGCC CTGAAAGAAA GAGATTAGGG AAAGTATTAG
AAATAAGATA AACAAAAAAG TATATTAAAA GAAGAAAGCA
TTTTTTAAAA TTACAAATGC AAAATTACCC TGATTTGGTC
AATATGTGTA CCCTGTTACT TCTCCCCTTC CTATGACATG
AACTTAACCA TAGAAAAGAA GGGGAAAGAA AACATCAAGG
GTCCCATAGA CTCACCCTGA AGTTCTCAGG ATCCACGTGC
AGCTTGTCAC AGTGCAGCTC ACTCAGctgG GCAAAGGTGC
CCTTGAGGTT GTCCAGGTGA GCCAGGCCAT CACTAAAGGC
ACCGAGCACT TTCTTGCCAT GAGCCTTCAC CTTAGGGTTG
CCCATAACAG CATCAGGAGT GGACAGATCC CCAAAGGACT
CAAAGAACCT CTGGGTCCAA GGGTAGACCA CCAGCAGCCT
AAGGGTGGGA AAATAGACCA ATAGGCAGAG AGAGTCAGTG
CCTATCAGAA ACCCAAGAGT CTTCTCTGTC TCCACATGCC
CAGTTTCTAT TGGTCTCCTT AAACCTGTCT TGTAACCTTG
ATACCAACCT GCCCAGGGCC TCACCACCAA CTTCATCCAC
GTTCACCTTG CCCCACAGGG CAGTAACGGC AGACTTCTCC
TCAGGAGTCA GGTGCACCAT GGTGTCTGTT TGAGGTTGCT
AGTGAACACA GTTGTGTCAG AAGCAAATGT

In certain embodiments, the human β^{A}-globin gene encoding a threonine to glutamine mutation at codon 87 (B4-1870) comprises the nucleotide sequence set forth in SEQ ID NO: 53, which is provided below.

[SEQ ID NO: 53]
gc aatgaaaata aatgtttttt attaggcaga atccagatgc
tcaaggccct tcataatatc ccccagttta gtagttggac
ttagggaaca aaggaacctt taatagaaat tggacagcaa
gaaagcgagc ttagtgatac ttgtgggcca gggcattagc
cacaccagcc accactttct gataggcagc ctgcactggt
ggggtgAATT CTTTGCCAAA GTGATGGGCC AGCACACAGA
CCAGCACGTT GCCCAGGAGC TGTGGGAGGA AGATAAGAGG
TATGAACATG ATTAGCAAAA GGGCCTAGCT TGGACTCAGA
ATAATCCAGC CTTATCCCAA CCATAAAATA AAAGCAGAAT
GGTAGCTGGA TTGTAGCTGC TATTAGCAAT ATGAAACCTC
TTACATCAGT TACAATTTAT ATGCAGAAAT ATTTATATGC
AGAAATATTG CTATTGCCTT AACCCAGAAA TTATCACTGT
TATTCTTTAG AATGGTGCAA AGAGGCATGA TACATTGTAT
CATTATTGCC CTGAAAGAAA GAGATTAGGG AAAGTATTAG
AAATAAGATA AACAAAAAAG TATATTAAAA GAAGAAAGCA
TTTTTTAAAA TTACAAATGC AAAATTACCC TGATTTGGTC
AATATGTGTA CCCTGTTACT TCTCCCCTTC CTATGACATG
AACTTAACCA TAGAAAAGAA GGGGAAAGAA AACATCAAGG
GTCCCATAGA CTCACCCTGA AGTTCTCAGG ATCCACGTGC
AGCTTGTCAC AGTGCAGCTC ACTCAGTTGG GCAAAGGTGC
CCTTGAGGTT GTCCAGGTGA GCCAGGCCAT CACTAAAGGC
ACCGAGCACT TTCTTGCCAT GAGCCTTCAC CTTAGGGTTG
CCCATAACAG CATCAGGAGT GGACAGATCC CCAAAGGACT
CAAAGAACCT CTGGGTCCAA GGGTAGACCA CCAGCAGCCT
AAGGGTGGGA AAATAGACCA ATAGGCAGAG AGAGTCAGTG
CCTATCAGAA ACCCAAGAGT CTTCTCTGTC TCCACATGCC
CAGTTTCTAT TGGTCTCCTT AAACCTGTCT TGTAACCTTG
ATACCAACCT GCCCAGGGCC TCACCACCAA CTTCATCCAC
GTTCACCTTG CCCCACAGGG CAGTAACGGC AGACTTCTCC
TCAGGAGTCA GGTGCACCAT GGTGTCTGTT TGAGGTTGCT
AGTGAACACA GTTGTGTCAG AAGCAAATGT

In certain non-limiting embodiments, the human β-globin gene is a human β^{A}-globin gene encoding a glutamic acid to alanine mutation at codon 22 (β^{A-E22A}). In certain embodiments, the human β^{A}-globin gene encoding a glutamic acid to alanine mutation at codon 22 (β^{A-E22A}) further comprises a deletion in intron 2 (e.g., an about 370 bp deletion). In certain embodiments, the human β^{A}-globin gene encoding a glutamic acid to alanine mutation at codon 22 (β^{A-E22A}) comprises the nucleotide sequence set forth in SEQ ID NO: 54, which is provided below:

[SEQ ID NO: 54]
gcaatgaaaataaatgttttttattaggcagaatccagatgctcaag
gcccttcataatatcccccagtttagtagttggacttagggaacaaa
ggaacctttaatagaaattggacagcaagaaagcgagcttagtgata
cttgtgggccagggcattagccacaccagccaccactttctgatagg
cagcctgcactggtggggtgAATTCTTTGCCAAAGTGATGGGCCAGC
ACACAGACCAGCACGTTGCCCAGGAGCTGTGGGAGGAAGATAAGAGG
TATGAACATGATTAGCAAAAGGGCCTAGCTTGGACTCAGAATAATCC
AGCCTTATCCCAACCATAAAATAAAAGCAGAATGGTAGCTGGATTGT
AGCTGCTATTAGCAATATGAAACCTCTTACATCAGTTACAATTTATA
TGCAGAAATATTTATATGCAGAAATATTGCTATTGCCTTAACCCAGA
AATTATCACTGTTATTCTTTAGAATGGTGCAAAGAGGCATGATACAT
TGTATCATTATTGCCCTGAAAGAAAGAGATTAGGGAAAGTATTAGAA
ATAAGATAAACAAAAAAGTATATTAAAAGAAGAAAGCATTTTTTAAA
ATTACAAATGCAAAATTACCCTGATTTGGTCAATATGTGTACCCTGT
TACTTCTCCCCTTCCTATGACATGAACTTAACCATAGAAAAGAAGGG
GAAAGAAAACATCAAGGGTCCCATAGACTCACCCTGAAGTTCTCAGG
ATCCACGTGCAGCTTGTCACAGTGCAGCTCACTCAGtgtGGCAAAGG
TGCCCTTGAGgttGTCCAGGTGAGCCAGGCCATCACTAAAGGCACCG
AGCACTTTCTTGCCATGAGCCTTCACCTTAGGGTTGCCCATAACAGC
ATCAGGAGTGGACAGATCCCCAAAGGACTCAAAGAACCTCTGGGTCC
AAGGGTAGACCACCAGCAGCCTAAGGGTGGGAAAATAGACCAATAGG
CAGAGAGAGTCAGTGCCTATCAGAAACCCAAGAGTCTTCTCTGTCTC
CACATGCCCAGTTTCTATTGGTCTCCTTAAACCTGTCTTGTAACCTT
GATACCAACCTGCCCAGGGCCTCACCACCAACggcATCCACGTTCAC
CTTGCCCCACAGGGCAGTAACGGCAGACTTCTCCTCAGGAGTCAGGT
GCACCATGGTGTCTGTTTGAGGTTGCTAGTGAACACAGTTGTGTCAG
AAGCAAATGT

In certain embodiments, the human β^{A}-globin gene encoding a glutamic acid to alanine mutation at codon 22 (β^{A-E22A}) comprises the nucleotide sequence set forth in SEQ ID NO: 55, which is provided below:

[SEQ ID NO: 55]
gcaatgaaaataaatgttttttattaggcagaatccagatgctcaag
gcccttcataatatcccccagtttagtagttggacttagggaacaaa
ggaacctttaatagaaattggacagcaagaaagcgagcttagtgata
cttgtgggccagggcattagccacaccagccaccactttctgatagg
cagcctgcactggtggggtgAATTCTTTGCCAAAGTGATGGGCCAGC
ACACAGACCAGCACGTTGCCCAGGAGCTGTGGGAGGAAGATAAGAGG
TATGAACATGATTAGCAAAAGGGCCTAGCTTGGACTCAGAATAATCC
AGCCTTATCCCAACCATAAAATAAAAGCAGAATGGTAGCTGGATTGT
AGCTGCTATTAGCAATATGAAACCTCTTACATCAGTTACAATTTATA
TGCAGAAATATTTATATGCAGAAATATTGCTATTGCCTTAACCCAGA
AATTATCACTGTTATTCTTTAGAATGGTGCAAAGAGGCATGATACAT
TGTATCATTATTGCCCTGAAAGAAAGAGATTAGGGAAAGTATTAGAA
ATAAGATAAACAAAAAAGTATATTAAAAGAAGAAAGCATTTTTTAAA
ATTACAAATGCAAAATTACCCTGATTTGGTCAATATGTGTACCCTGT
TACTTCTCCCCTTCCTATGACATGAACTTAACCATAGAAAAGAAGGG
GAAAGAAAACATCAAGGGTCCCATAGACTCACCCTGAAGTTCTCAGG
ATCCACGTGCAGCTTGTCACAGTGCAGCTCACTCAGtgtGGCAAAGG
TGCCCTTGAGgttGTCCAGGTGAGCCAGGCCATCACTAAAGGCACCG
AGCACTTTCTTGCCATGAGCCTTCACCTTAGGGTTGCCCATAACAGC
ATCAGGAGTGGACAGATCCCCAAAGGACTCAAAGAACCTCTGGGTCC
AAGGGTAGACCACCAGCAGCCTAAGGGTGGGAAAATAGACCAATAGG
CAGAGAGAGTCAGTGCCTATCAGAAACCCAAGAGTCTTCTCTGTCTC
CACATGCCCAGTTTCTATTGGTCTCCTTAAACCTGTCTTGTAACCTT
GATACCAACCTGCCCAGGGCCTCACCACCAACTGCATCCACGTTCAC
CTTGCCCCACAGGGCAGTAACGGCAGACTTCTCCTCAGGAGTCAGGT
GCACCATGGTGTCTGTTTGAGGTTGCTAGTGAACACAGTTGTGTCAG
AAGCAAATGT

In certain embodiments, the human β^{A}-globin gene encoding a glutamic acid to alanine mutation at codon 22 (β^{A-E22A}) comprises the nucleotide sequence set forth in SEQ ID NO: 56, which is provided below:

[SEQ ID NO: 56]
gcaatgaaaataaatgttttttattaggcagaatccagatgctcaaggcc
cttcataatatcccccagtttagtagttggacttagggaacaaaggaacc
tttaatagaaattggacagcaagaaagcgagcttagtgatacttgtgggc
cagggcattagccacaccagccaccactttctgataggcagcctgcactg
AgtggggtgAATTCTTTGCCAAAGTGATGGGCCAGCACACGACCAGCACG
TTGCCCAGGAGCTGTGGGAGGAAGATAAGAGGTATGAACATGATTAGCAA
AAGGGCCTAGCTTGGACTCAGAATAATCCAGCCTTATCCCAACCATAAAA
TAAAAGCAGAATGGTAGCTGGATTGTAGCTGCTATTAGCAATATGAAACC
TCTTACATCAGTTACAATTTATATGCAGAAATATTTATATGCAGAAATAT
TGCTATTGCCTTAACCCAGAAATTATCACTGTTATTCTTTAGAATGGTGC
AAAGAGGCATGATACATTGTATCATTATTGCCCTGAAAGAAAGAGATTAG
GGAAAGTATTAGAAATAAGATAAACAAAAAAGTATATTAAAAGAAGAAAG
CATTTTTTAAAATTACAAATGCAAAATTACCCTGATTTGGTCAATATGTG
TACCCTGTTACTTCTCCCCTTCCTATGACATGAACTTAACCATAGAAAAG
AAGGGGAAAGAAAACATCAAGGGTCCCATAGACTCACCCTGAAGTTCTCA
GGATCCACGTGCAGCTTGTCACAGTGCAGCTCACTCAGtgtGGCAAAGGT
GCCCTTGAGgttGTCCAGGTGAGCCAGGCCATCACTAAAGGCACCGAGCA
CTTTCTTGCCATGAGCCTTCACCTTAGGGTTGCCCATAACAGCATCAGGA
GTGGACAGATCCCCAAAGGACTCAAAGAACCTCTGGGTCCAAGGGTAGAC
CACCAGCAGCCTAAGGGTGGGAAAATAGACCAATAGGCAGAGAGAGTCAG
TGCCTATCAGAAACCCAAGAGTCTTCTCTGTCTCCACATGCCCAGTTTCT
ATTGGTCTCCTTAAACCTGTCTTGTAACCTTGATACCAACCTGCCCAGGG
CCTCACCACCAACAGCATCCACGTTCACCTTGCCCCACAGGGCAGTAACG
GCAGACTTCTCCTCAGGAGTCAGGTGCACCATGGTGTCTGTTTGAGGTTG
CTAGTGAACACAGTTGTGTCAGAAGCAAATGT

In certain embodiments, the human β^{A}-globin gene encoding a glutamic acid to alanine mutation at codon 22 (β^{A-E22A}) comprises the nucleotide sequence set forth in SEQ ID NO: 57, which is provided below:

[SEQ ID NO: 57]
gcaatgaaaataaatgttttttattaggcagaatccagatgctcaaggccc
ttcataatatcccccagtttagtagttggacttagggaacaaaggaacctt
taatagaaattggacagcaagaaagcgagcttagtgatacttgtgggccag
ggcattagccacaccagccaccactttctgataggcagcctgcactggtgg
ggtgAATTCTTTGCCAAAGTGATGGGCCAGCACACAGACCAGCACGTTGCC
CAGGAGCTGTGGGAGGAAGATAAGAGGTATGAACATGATTAGCAAAAGGGC
CTAGCTTGGACTCAGAATAATCCAGCCTTATCCCAACCATAAAATAAAAGC
AGAATGGTAGCTGGATTGTAGCTGCTATTAGCAATATGAAACCTCTTACAT
CAGTTACAATTTATATGCAGAAATATTTATATGCAGAAATATTGCTATTGC
CTTAACCCAGAAATTATCACTGTTATTCTTTAGAATGGTGCAAAGAGGCAT
GATACATTGTATCATTATTGCCCTGAAAGAAAGAGATTAGGGAAAGTATTA
GAAATAAGATAAACAAAAAAGTATATTAAAAGAAGAAAGCATTTTTTAAAA
TTACAAATGCAAAATTACCCTGATTTGGTCAATATGTGTACCCTGTTACTT
CTCCCCTTCCTATGACATGAACTTAACCATAGAAAAGAAGGGGAAAGAAAA
CATCAAGGGTCCCATAGACTCACCCTGAAGTTCTCAGGATCCACGTGCAGC
TTGTCACAGTGCAGCTCACTCAGtgtGGCAAAGGTGCCCTTGAGgttGTCC
AGGTGAGCCAGGCCATCACTAAAGGCACCGAGCACTTTCTTGCCATGAGCC
TTCACCTTAGGGTTGCCCATAACAGCATCAGGAGTGGACAGATCCCCAAAG
GACTCAAAGAACCTCTGGGTCCAAGGGTAGACCACCAGCAGCCTAAGGGTG
GGAAAATAGACCAATAGGCAGAGAGAGTCAGTGCCTATCAGAAACCCAAGA
GTCTTCTCTGTCTCCACATGCCCAGTTTCTATTGGTCTCCTTAAACCTGTC
TTGTAACCTTGATACCAACCTGCCCAGGGCCTCACCACCAACCGCATCCAC
GTTCACCTTGCCCCACAGGGCAGTAACGGCAGACTTCTCCTCAGGAGTCAG
GTGCACCATGGTGTCTGTTTGAGGTTGCTAGTGAACACAGTTGTGTCAGAA
GCAAATGT

In certain non-limiting embodiments, the human β-globin gene is a human β^{A}-globin gene encoding an asparagine to lysine mutation at codon 80 (β^{-N80K}). In certain embodiments, the human β^{A}-globin gene encoding an asparagine to lysine mutation at codon 80 (β^{-N80K}) further comprises a deletion in intron 2 (e.g., an about 370 bp deletion). In certain embodiments, the human β^{A}-globin gene encoding an asparagine to lysine mutation at codon 80 (β^{-N80K}) comprises the nucleotide sequence set forth in SEQ ID NO: 58, which is provided below:

[SEQ ID NO: 58]
gcaatgaaaataaatgttttttattaggcagaatccagatgctcaaggcc
cttcataatatcccccagtttagtagttggacttagggaacaaaggaacc
tttaatagaaattggacagcaagaaagcgagcttagtgatacttgtgggc
cagggcattagccacaccagccaccactttctgataggcagcctgcactg
gtggggtgAATTCTTTGCCAAAGTGATGGGCCAGCACACAGACCAGCACG
TTGCCCAGGAGCTGTGGGAGGAAGATAAGAGGTATGAACATGATTAGCAA
AAGGGCCTAGCTTGGACTCAGAATAATCCAGCCTTATCCCAACCATAAAA
TAAAAGCAGAATGGTAGCTGGATTGTAGCTGCTATTAGCAATATGAAACC
TCTTACATCAGTTACAATTTATATGCAGAAATATTTATATGCAGAAATAT
TGCTATTGCCTTAACCCAGAAATTATCACTGTTATTCTTTAGAATGGTGC
AAAGAGGCATGATACATTGTATCATTATTGCCCTGAAAGAAAGAGATTAG
GGAAAGTATTAGAAATAAGATAAACAAAAAAGTATATTAAAAGAAGAAAG
CATTTTTTAAAATTACAAATGCAAAATTACCCTGATTTGGTCAATATGTG
TACCCTGTTACTTCTCCCCTTCCTATGACATGAACTTAACCATAGAAAAG
AAGGGGAAAGAAAACATCAAGGGTCCCATAGACTCACCCTGAAGTTCTCA
GGATCCACGTGCAGCTTGTCACAGTGCAGCTCACTCAGtgtGGCAAAGGT
GCCCTTGAGcttGTCCAGGTGAGCCAGGCCATCACTAAAGGCACCGAGCA
CTTTCTTGCCATGAGCCTTCACCTTAGGGTTGCCCATAACAGCATCAGGA
GTGGACAGATCCCCAAAGGACTCAAAGAACCTCTGGGTCCAAGGGTAGAC
CACCAGCAGCCTAAGGGTGGGAAAATAGACCAATAGGCAGAGAGAGTCAG
TGCCTATCAGAAACCCAAGAGTCTTCTCTGTCTCCACATGCCCAGTTTCT
ATTGGTCTCCTTAAACCTGTCTTGTAACCTTGATACCAACCTGCCCAGGG
CCTCACCACCAACttcATCCACGTTCACCTTGCCCCACAGGGCAGTAACG
GCAGACTTCTCCTCAGGAGTCAGGTGCACCATGGTGTCTGTTTGAGGTTG
CTAGTGAACACAGTTGTGTCAGAAGCAAATGT

In certain embodiments, the human β^{A}-globin gene encoding an asparagine to lysine mutation at codon 80 (β^{-N80K}) comprises the nucleotide sequence set forth in SEQ ID NO: 59, which is provided below:

[SEQ ID NO: 59]
gcaatgaaaataaatgttttttattaggcagaatccagatgctcaaggcc
cttcataatatcccccagtttagtagttggacttagggaacaaaggaacc
tttaatagaaattggacagcaagaaagcgagcttagtgatacttgtgggc
cagggcattagccacaccagccaccactttctgataggcagcctgcactg
gtggggtgAATTCTTTGCCAAAGTGATGGGCCAGCACACAGACCAGCACG
TTGCCCAGGAGCTGTGGGAGGAAGATAAGAGGTATGAACATGATTAGCAA
AAGGGCCTAGCTTGGACTCAGAATAATCCAGCCTTATCCCAACCATAAAA
TAAAAGCAGAATGGTAGCTGGATTGTAGCTGCTATTAGCAATATGAAACC
TCTTACATCAGTTACAATTTATATGCAGAAATATTTATATGCAGAAATAT
TGCTATTGCCTTAACCCAGAAATTATCACTGTTATTCTTTAGAATGGTGC
AAAGAGGCATGATACATTGTATCATTATTGCCCTGAAAGAAAGAGATTAG
GGAAAGTATTAGAAATAAGATAAACAAAAAAGTATATTAAAAGAAGAAAG
CATTTTTTAAAATTACAAATGCAAAATTACCCTGATTTGGTCAATATGTG
TACCCTGTTACTTCTCCCCTTCCTATGACATGAACTTAACCATAGAAAAG
AAGGGGAAAGAAAACATCAAGGGTCCCATAGACTCACCCTGAAGTTCTCA
GGATCCACGTGCAGCTTGTCACAGTGCAGCTCACTCAGtgtGGCAAAGGT
GCCCTTGAGTTTGTCCAGGTGAGCCAGGCCATCACTAAAGGCACCGAGCA
CTTTCTTGCCATGAGCCTTCACCTTAGGGTTGCCCATAACAGCATCAGGA
GTGGACAGATCCCCAAAGGACTCAAAGAACCTCTGGGTCCAAGGGTAGAC
CACCAGCAGCCTAAGGGTGGGAAAATAGACCAATAGGCAGAGAGAGTCAG
TGCCTATCAGAAACCCAAGAGTCTTCTCTGTCTCCACATGCCCAGTTTCT
ATTGGTCTCCTTAAACCTGTCTTGTAACCTTGATACCAACCTGCCCAGGG
CCTCACCACCAACttcATCCACGTTCACCTTGCCCCACAGGGCAGTAACG
GCAGACTTCTCCTCAGGAGTCAGGTGCACCATGGTGTCTGTTTGAGGTTG
CTAGTGAACACAGTTGTGTCAGAAGCAAATGT

In certain non-limiting embodiments, the human β-globin gene is human β^{A}-globin gene encoding a glutamic acid to alanine mutation at codon 22 and an asparagine to lysine mutation at codon 80. In certain embodiments, the human β^{A}-globin gene encoding a glutamic acid to alanine mutation at codon 22 and an asparagine to lysine mutation at codon 80 further comprises a deletion in intron 2 (e.g., an about 370 bp deletion). In certain embodiments, the human β^{A}-globin gene encoding a glutamic acid to alanine mutation at codon 22 and an asparagine to lysine mutation at codon 80 comprises the nucleotide sequence set forth in SEQ ID NO: 60, which is provided below:

[SEQ ID NO: 60]
gcaatgaaaataaatgttttttattaggcagaatccagatgctcaaggcc
cttcataatatcccccagtttagtagttggacttagggaacaaaggaacc
tttaatagaaattggacagcaagaaagcgagcttagtgatacttgtgggc
cagggcattaGCCACACCAGCCACCACTTTCTGATAGGCAGCCTGCACTG
GTGGGGTGAATTCTTTGCCAAAGTGATGGGCCAGCACACAGACCAGCACG
TTGCCCAGGAGCTGTGGGAGGAAGATAAGAGGTATGAACATGATTAGCAA
AAGGGCCTAGCTTGGACTCAGAATAATCCAGCCTTATCCCAACCATAAAA
TAAAAGCAGAATGGTAGCTGGATTGTAGCTGCTATTAGCAATATGAAACC
TCTTACATCAGTTACAATTTATATGCAGAAATATTTATATGCAGAAATAT
TGCTATTGCCTTAACCCAGAAATTATCACTGTTATTCTTTAGAATGGTGC
AAAGAGGCATGATACATTGTATCATTATTGCCCTGAAAGAAAGAGATTAG
GGAAAGTATTAGAAATAAGATAAACAAAAAAGTATATTAAAAGAAGAAAG
CATTTTTTAAAATTACAAATGCAAAATTACCCTGATTTGGTCAATATGTG
TACCCTGTTACTTCTCCCCTTCCTATGACATGAACTTAACCATAGAAAAG
AAGGGGAAAGAAAACATCAAGGGTCCCATAGACTCACCCTGAAGTTCTCA
GGATCCACGTGCAGCTTGTCACAGTGCAGCTCACTCAGTGTGGCAAAGGT
GCCCTTGAGCTTGTCCAGGTGAGCCAGGCCATCACTAAAGGCACCGAGCA
CTTTCTTGCCATGAGCCTTCACCTTAGGGTTGCCCATAACAGCATCAGGA
GTGGACAGATCCCCAAAGGACTCAAAGAACCTCTGGGTCCAAGGGTAGAC
CACCAGCAGCCTAAGGGTGGGAAAATAGACCAATAGGCAGAGAGAGTCAG
TGCCTATCAGAAACCCAAGAGTCTTCTCTGTCTCCACATGCCCAGTTTCT
ATTGGTCTCCTTAAACCTGTCTTGTAACCTTGATACCAACCTGCCCAGGG
CCTCACCACCAACGGCATCCACGTTCACCTTGCCCCACAGGGCAGTAACG
GCAGACTTCTCCTCAGGAGTCAGGTGCACCATGGTGTCTGTTTGAGGTTG
CTAGTGAACACAGTTGTGTCAGAAGCAAATGT

In certain embodiments, the human β^{A}-globin gene encoding a glutamic acid to alanine mutation at codon 22 and an asparagine to lysine mutation at codon 80 comprises the nucleotide sequence set forth in SEQ ID NO: 61, which is provided below:

[SEQ ID NO: 61]
gcaatgaaaataaatgttttttattaggcagaatccagatgctcaaggccc
ttcataatatcccccagtttagtagttggacttagggaacaaaggaacctt
taatagaaattggacagcaagaaagcgagcttagtgatacttgtgggccag
ggcattagccacaccagccaccactttctgataggcagcctgcactggtgg
ggtgAATTCTTTGCCAAAGTGATGGGCCAGCACACAGACCAGCACGTTGCC
CAGGAGCTGTGGGAGGAAGATAAGAGGTATGAACATGATTAGCAAAAGGGC
CTAGCTTGGACTCAGAATAATCCAGCCTTATCCCAACCATAAAATAAAAGC
AGAATGGTAGCTGGATTGTAGCTGCTATTAGCAATATGAAACCTCTTACAT
CAGTTACAATTTATATGCAGAAATATTTATATGCAGAAATATTGCTATTGC
CTTAACCCAGAAATTATCACTGTTATTCTTTAGAATGGTGCAAAGAGGCAT
GATACATTGTATCATTATTGCCCTGAAAGAAAGAGATTAGGGAAAGTATTA
GAAATAAGATAAACAAAAAAGTATATTAAAAGAAGAAAGCATTTTTTAAAA
TTACAAATGCAAAATTACCCTGATTTGGTCAATATGTGTACCCTGTTACTT
CTCCCCTTCCTATGACATGAACTTAACCATAGAAAAGAAGGGGAAAGAAAA
CATCAAGGGTCCCATAGACTCACCCTGAAGTTCTCAGGATCCACGTGCAGC
TTGTCACAGTGCAGCTCACTCAGtgtGGCAAAGGTGCCCTTGAGTttGTCC
AGGTGAGCCAGGCCATCACTAAAGGCACCGAGCACTTTCTTGCCATGAGCC
TTCACCTTAGGGTTGCCCATAACAGCATCAGGAGTGGACAGATCCCCAAAG
GACTCAAAGAACCTCTGGGTCCAAGGGTAGACCACCAGCAGCCTAAGGGTG
GGAAAATAGACCAATAGGCAGAGAGAGTCAGTGCCTATCAGAAACCCAAGA
GTCTTCTCTGTCTCCACATGCCCAGTTTCTATTGGTCTCCTTAAACCTGTC
TTGTAACCTTGATACCAACCTGCCCAGGGCCTCACCACCAACggcATCCAC
GTTCACCTTGCCCCACAGGGCAGTAACGGCAGACTTCTCCTCAGGAGTCAG
GTGCACCATGGTGTCTGTTTGAGGTTGCTAGTGAACACAGTTGTGTCAGAA
GCAAATGT

In certain embodiments, the human β^{A}-globin gene encoding a glutamic acid to alanine mutation at codon 22 and an asparagine to lysine mutation at codon 80 comprises the nucleotide sequence set forth in SEQ ID NO: 62, which is provided below:

[SEQ ID NO: 62]
GCAATGAAAATAAATGTTTTTTATTAGGCAGAATCCAGATGCTCAAGGCC
CTTCATAATATCCCCCAGTTTAGTAGTTGGACTTAGGGAACAAAGGAACC
TTTAATAGAAATTGGACAGCAAGAAAGCGAGCTTAGTGATACTTGTGGGC
CAGGGCATTAGCCACACCAGCCACCACTTTCTGATAGGCAGCCTGCACTG
GTGGGGTGAATTCTTTGCCAAAGTGATGGGCCAGCACACAGACCAGCACG
TTGCCCAGGAGCTGTGGGAGGAAGATAAGAGGTATGAACATGATTAGCAA
AAGGGCCTAGCTTGGACTCAGAATAATCCAGCCTTATCCCAACCATAAAA
TAAAAGCAGAATGGTAGCTGGATTGTAGCTGCTATTAGCAATATGAAACC
TCTTACATCAGTTACAATTTATATGCAGAAATATTTATATGCAGAAATAT
TGCTATTGCCTTAACCCAGAAATTATCACTGTTATTCTTTAGAATGGTGC
AAAGAGGCATGATACATTGTATCATTATTGCCCTGAAAGAAAGAGATTAG
GGAAAGTATTAGAAATAAGATAAACAAAAAAGTATATTAAAAGAAGAAAG
CATTTTTTAAAATTACAAATGCAAAATTACCCTGATTTGGTCAATATGTG
TACCCTGTTACTTCTCCCCTTCCTATGACATGAACTTAACCATAGAAAAG
AAGGGGAAAGAAAACATCAAGGGTCCCATAGACTCACCCTGAAGTTCTCA
GGATCCACGTGCAGCTTGTCACAGTGCAGCTCACTCAGTGTGGCAAAGGT
GCCCTTGAGCTTGTCCAGGTGAGCCAGGCCATCACTAAAGGCACCGAGCA
CTTTCTTGCCATGAGCCTTCACCTTAGGGTTGCCCATAACAGCATCAGGA
GTGGACAGATCCCCAAAGGACTCAAAGAACCTCTGGGTCCAAGGGTAGAC
CACCAGCAGCCTAAGGGTGGGAAAATAGACCAATAGGCAGAGAGAGTCAG
TGCCTATCAGAAACCCAAGAGTCTTCTCTGTCTCCACATGCCCAGTTTCT
ATTGGTCTCCTTAAACCTGTCTTGTAACCTTGATACCAACCTGCCCAGGG
CCTCACCACCAACTGCATCCACGTTCACCTTGCCCCACAGGGCAGTAACG
GCAGACTTCTCCTCAGGAGTCAGGTGCACCATGGTGTCTGTTTGAGGTTG
CTAGTGAACACAGTTGTGTCAGAAGCAAATGT

In certain embodiments, the human BA_globin gene encoding a glutamic acid to alanine mutation at codon 22 and an asparagine to lysine mutation at codon 80 comprises the nucleotide sequence set forth in SEQ ID NO: 63, which is provided below:

[SEQ ID NO: 63]
gcaatgaaaataaatgttttttattaggcagaatccagatgctcaaggccc
ttcataatatcccccagtttagtagttggacttagggaacaaaggaacctt
taatagaaattggacagcaagaaagcgagcttagtgatacttgtgggccag
ggcattagccacaccagccaccactttctgataggcagcctgcactggtgg
ggtgAATTCTTTGCCAAAGTGATGGGCCAGCACACAGACCAGCACGTTGCC
CAGGAGCTGTGGGAGGAAGATAAGAGGTATGAACATGATTAGCAAAAGGGC
CTAGCTTGGACTCAGAATAATCCAGCCTTATCCCAACCATAAAATAAAAGC
AGAATGGTAGCTGGATTGTAGCTGCTATTAGCAATATGAAACCTCTTACAT
CAGTTACAATTTATATGCAGAAATATTTATATGCAGAAATATTGCTATTGC
CTTAACCCAGAAATTATCACTGTTATTCTTTAGAATGGTGCAAAGAGGCAT
GATACATTGTATCATTATTGCCCTGAAAGAAAGAGATTAGGGAAAGTATTA
GAAATAAGATAAACAAAAAAGTATATTAAAAGAAGAAAGCATTTTTTAAAA
TTACAAATGCAAAATTACCCTGATTTGGTCAATATGTGTACCCTGTTACTT
CTCCCCTTCCTATGACATGAACTTAACCATAGAAAAGAAGGGGAAAGAAAA
CATCAAGGGTCCCATAGACTCACCCTGAAGTTCTCAGGATCCACGTGCAGC
TTGTCACAGTGCAGCTCACTCAGTGTGGCAAAGGTGCCCTTGAGCTTGTCC
AGGTGAGCCAGGCCATCACTAAAGGCACCGAGCACTTTCTTGCCATGAGCC
TTCACCTTAGGGTTGCCCATAACAGCATCAGGAGTGGACAGATCCCCAAAG
GACTCAAAGAACCTCTGGGTCCAAGGGTAGACCACCAGCAGCCTAAGGGTG
GGAAAATAGACCAATAGGCAGAGAGAGTCAGTGCCTATCAGAAACCCAAGA
GTCTTCTCTGTCTCCACATGCCCAGTTTCTATTGGTCTCCTTAAACCTGTC
TTGTAACCTTGATACCAACCTGCCCAGGGCCTCACCACCAACAGCATCCAC
GTTCACCTTGCCCCACAGGGCAGTAACGGCAGACTTCTCCTCAGGAGTCAG
GTGCACCATGGTGTCTGTTTGAGGTTGCTAGTGAACACAGTTGTGTCAGAA
GCAAATGT

In certain embodiments, the human β^{A}-globin gene encoding a glutamic acid to alanine mutation at codon 22 and an asparagine to lysine mutation at codon 80 comprises the nucleotide sequence set forth in SEQ ID NO: 64, which is provided below:

[SEQ ID NO: 64]
gcaatgaaaataaatgttttttattaggcagaatccagatgctcaaggccc
ttcataatatcccccagtttagtagttggacttagggaacaaaggaacctt
taatagaaattggacagcaagaaagcgagcttagtgatacttgtgggccag
ggcattagccacaccagccaccactttctgataggcagcctgcactggtgg
ggtgAATTCTTTGCCAAAGTGATGGGCCAGCACACAGACCAGCACGTTGCC
CAGGAGCTGTGGGAGGAAGATAAGAGGTATGAACATGATTAGCAAAAGGGC
CTAGCTTGGACTCAGAATAATCCAGCCTTATCCCAACCATAAAATAAAAGC
AGAATGGTAGCTGGATTGTAGCTGCTATTAGCAATATGAAACCTCTTACAT
CAGTTACAATTTATATGCAGAAATATTTATATGCAGAAATATTGCTATTGC
CTTAACCCAGAAATTATCACTGTTATTCTTTAGAATGGTGCAAAGAGGCAT
GATACATTGTATCATTATTGCCCTGAAAGAAAGAGATTAGGGAAAGTATTA
GAAATAAGATAAACAAAAAAGTATATTAAAAGAAGAAAGCATTTTTTAAAA
TTACAAATGCAAAATTACCCTGATTTGGTCAATATGTGTACCCTGTTACTT
CTCCCCTTCCTATGACATGAACTTAACCATAGAAAAGAAGGGGAAAGAAAA
CATCAAGGGTCCCATAGACTCACCCTGAAGTTCTCAGGATCCACGTGCAGC
TTGTCACAGTGCAGCTCACTCAGTGTGGCAAAGGTGCCCTTGAGCTTGTCC
AGGTGAGCCAGGCCATCACTAAAGGCACCGAGCACTTTCTTGCCATGAGCC
TTCACCTTAGGGTTGCCCATAACAGCATCAGGAGTGGACAGATCCCCAAAG
GACTCAAAGAACCTCTGGGTCCAAGGGTAGACCACCAGCAGCCTAAGGGTG
GGAAAATAGACCAATAGGCAGAGAGAGTCAGTGCCTATCAGAAACCCAAGA
GTCTTCTCTGTCTCCACATGCCCAGTTTCTATTGGTCTCCTTAAACCTGTC
TTGTAACCTTGATACCAACCTGCCCAGGGCCTCACCACCAACCGCATCCAC
GTTCACCTTGCCCCACAGGGCAGTAACGGCAGACTTCTCCTCAGGAGTCAG
GTGCACCATGGTGTCTGTTTGAGGTTGCTAGTGAACACAGTTGTGTCAGAA
GCAAATGT

In certain embodiments, the human β^{A}-globin gene encoding a glutamic acid to alanine mutation at codon 22 and an asparagine to lysine mutation at codon 80 comprises the nucleotide sequence set forth in SEQ ID NO: 65, which is provided below:

[SEQ ID NO: 65]
gcaatgaaaataaatgttttttattaggcagaatccagatgctcaaggcc
cttcataatatcccccagtttagtagttggacttagggaacaaaggaacc
tttaatagaaattggacagcaagaaagcgagcttagtgatacttgtgggc
cagggcattagccacaccagccaccactttctgataggcagcctgcactg
gtggggtgAATTCTTTGCCAAAGTGATGGGCCAGCACACAGACCAGCACG
TTGCCCAGGAGCTGTGGGAGGAAGATAAGAGGTATGAACATGATTAGCAA
AAGGGCCTAGCTTGGACTCAGAATAATCCAGCCTTATCCCAACCATAAAA
TAAAAGCAGAATGGTAGCTGGATTGTAGCTGCTATTAGCAATATGAAACC
TCTTACATCAGTTACAATTTATATGCAGAAATATTTATATGCAGAAATAT
TGCTATTGCCTTAACCCAGAAATTATCACTGTTATTCTTTAGAATGGTGC
AAAGAGGCATGATACATTGTATCATTATTGCCCTGAAAGAAAGAGATTAG
GGAAAGTATTAGAAATAAGATAAACAAAAAAGTATATTAAAAGAAGAAAG
CATTTTTTAAAATTACAAATGCAAAATTACCCTGATTTGGTCAATATGTG
TACCCTGTTACTTCTCCCCTTCCTATGACATGAACTTAACCATAGAAAAG
AAGGGGAAAGAAAACATCAAGGGTCCCATAGACTCACCCTGAAGTTCTCA
GGATCCACGTGCAGCTTGTCACAGTGCAGCTCACTCAGTGTGGCAAAGGT
GCCCTTGAGTTTGTCCAGGTGAGCCAGGCCATCACTAAAGGCACCGAGCA
CTTTCTTGCCATGAGCCTTCACCTTAGGGTTGCCCATAACAGCATCAGGA
GTGGACAGATCCCCAAAGGACTCAAAGAACCTCTGGGTCCAAGGGTAGAC
CACCAGCAGCCTAAGGGTGGGAAAATAGACCAATAGGCAGAGAGAGTCAG
TGCCTATCAGAAACCCAAGAGTCTTCTCTGTCTCCACATGCCCAGTTTCT
ATTGGTCTCCTTAAACCTGTCTTGTAACCTTGATACCAACCTGCCCAGGG
CCTCACCACCAACTGCATCCACGTTCACCTTGCCCCACAGGGCAGTAACG
GCAGACTTCTCCTCAGGAGTCAGGTGCACCATGGTGTCTGTTTGAGGTTG
CTAGTGAACACAGTTGTGTCAGAAGCAAATGT

In certain embodiments, the human β^{A}-globin gene encoding a glutamic acid to alanine mutation at codon 22 and an asparagine to lysine mutation at codon 80 comprises the nucleotide sequence set forth in SEQ ID NO: 66, which is provided below:

[SEQ ID NO: 66]
gcaatgaaaataaatgttttttattaggcagaatccagatgctcaaggccc
ttcataatatcccccagtttagtagttggACTTAGGGAACAAAGGAACCTT
TAATAGAAATTGGACAGCAAGAAAGCGAGCTTAGTGATACTTGTGGGCCAG
GGCATTAGCCACACCAGCCACCACTTTCTGATAGGCAGCCTGCACTGGTGG
GGTGAATTCTTTGCCAAAGTGATGGGCCAGCACACAGACCAGCACGTTGCC
CAGGAGCTGTGGGAGGAAGATAAGAGGTATGAACATGATTAGCAAAAGGGC
CTAGCTTGGACTCAGAATAATCCAGCCTTATCCCAACCATAAAATAAAAGC
AGAATGGTAGCTGGATTGTAGCTGCTATTAGCAATATGAAACCTCTTACAT
CAGTTACAATTTATATGCAGAAATATTTATATGCAGAAATATTGCTATTGC
CTTAACCCAGAAATTATCACTGTTATTCTTTAGAATGGTGCAAAGAGGCAT
GATACATTGTATCATTATTGCCCTGAAAGAAAGAGATTAGGGAAAGTATTA
GAAATAAGATAAACAAAAAAGTATATTAAAAGAAGAAAGCATTTTTTAAAA
TTACAAATGCAAAATTACCCTGATTTGGTCAATATGTGTACCCTGTTACTT
CTCCCCTTCCTATGACATGAACTTAACCATAGAAAAGAAGGGGAAAGAAAA
CATCAAGGGTCCCATAGACTCACCCTGAAGTTCTCAGGATCCACGTGCAGC
TTGTCACAGTGCAGCTCACTCAGTGTGGCAAAGGTGCCCTTGAGTTTGTCC
AGGTGAGCCAGGCCATCACTAAAGGCACCGAGCACTTTCTTGCCATGAGCC
TTCACCTTAGGGTTGCCCATAACAGCATCAGGAGTGGACAGATCCCCAAAG
GACTCAAAGAACCTCTGGGTCCAAGGGTAGACCACCAGCAGCCTAAGGGTG
GGAAAATAGACCAATAGGCAGAGAGAGTCAGTGCCTATCAGAAACCCAAGA
GTCTTCTCTGTCTCCACATGCCCAGTTTCTATTGGTCTCCTTAAACCTGTC
TTGTAACCTTGATACCAACCTGCCCAGGGCCTCACCACCAACAGCATCCAC
GTTCACCTTGCCCCACAGGGCAGTAACGGCAGACTTCTCCTCAGGAGTCAG
GTGCACCATGGTGTCTGTTTGAGGTTGCTAGTGAACACAGTTGTGTCAGAA
GCAAATGT

In certain embodiments, the human β^{A}-globin gene encoding a glutamic acid to alanine mutation at codon 22 and an asparagine to lysine mutation at codon 80 comprises the nucleotide sequence set forth in SEQ ID NO: 67, which is provided below:

[SEQ ID NO: 67]
gcaatgaaaataaatgttttttattaggcagaatccagatgctcaaggcc
cttcataatatcccccagtttagtagttggacttagggaacaaaggaacc
tttaatagaaattggacagcaagaaagcgagcttagtgatacttgtgggc
cagggcattagccacaccagccaccactttctgataggcagcctgcactg
gtggggtgAATTCTTTGCCAAAGTGATGGGCCAGCACACAGACCAGCACG
TTGCCCAGGAGCTGTGGGAGGAAGATAAGAGGTATGAACATGATTAGCAA
AAGGGCCTAGCTTGGACTCAGAATAATCCAGCCTTATCCCAACCATAAAA
TAAAAGCAGAATGGTAGCTGGATTGTAGCTGCTATTAGCAATATGAAACC
TCTTACATCAGTTACAATTTATATGCAGAAATATTTATATGCAGAAATAT
TGCTATTGCCTTAACCCAGAAATTATCACTGTTATTCTTTAGAATGGTGC
AAAGAGGCATGATACATTGTATCATTATTGCCCTGAAAGAAAGAGATTAG
GGAAAGTATTAGAAATAAGATAAACAAAAAAGTATATTAAAAGAAGAAAG
CATTTTTTAAAATTACAAATGCAAAATTACCCTGATTTGGTCAATATGTG
TACCCTGTTACTTCTCCCCTTCCTATGACATGAACTTAACCATAGAAAAG
AAGGGGAAAGAAAACATCAAGGGTCCCATAGACTCACCCTGAAGTTCTCA
GGATCCACGTGCAGCTTGTCACAGTGCAGCTCACTCAGTGTGGCAAAGGT
GCCCTTGAGTTTGTCCAGGTGAGCCAGGCCATCACTAAAGGCACCGAGCA
CTTTCTTGCCATGAGCCTTCACCTTAGGGTTGCCCATAACAGCATCAGGA
GTGGACAGATCCCCAAAGGACTCAAAGAACCTCTGGGTCCAAGGGTAGAC
CACCAGCAGCCTAAGGGTGGGAAAATAGACCAATAGGCAGAGAGAGTCAG
TGCCTATCAGAAACCCAAGAGTCTTCTCTGTCTCCACATGCCCAGTTTCT
ATTGGTCTCCTTAAACCTGTCTTGTAACCTTGATACCAACCTGCCCAGGG
CCTCACCACCAACCGCATCCACGTTCACCTTGCCCCACAGGGCAGTAACG
GCAGACTTCTCCTCAGGAGTCAGGTGCACCATGGTGTCTGTTTGAGGTTG
CTAGTGAACACAGTTGTGTCAGAAGCAAATGT

In certain non-limiting embodiments, the human β-globin gene is a human β^{A}-globin gene encoding a glutamic acid to alanine mutation at codon 22 and a threonine to glutamine mutation at codon 87. In certain embodiments, the human β^{A}-globin gene encoding a glutamic acid to alanine mutation at codon 22 and a threonine to glutamine mutation at codon 87 further comprises a deletion in intron 2 (e.g., an about 370 bp deletion). In certain embodiments, the human β^{A}-globin gene encoding a glutamic acid to alanine mutation at codon 22 and a threonine to glutamine mutation at codon 87 comprises the nucleotide sequence set forth in SEQ ID NO: 68, which is provided below:

[SEQ ID NO: 68]
gcaatgaaaataaatgttttttattaggcagaatccagatgctcaaggcc
cttcataatatcccccagtttagtagttggacttagggaacaaaggaacc
tttaatagaaattggacagcaagaaagcgagcttagtgatacttgtgggc
cagggcattagccacaccagccaccactttctgataggcagcctgcactg
gtggggtgAATTCTTTGCCAAAGTGATGGGCCAGCACACAGACCAGCACG
TTGCCCAGGAGCTGTGGGAGGAAGATAAGAGGTATGAACATGATTAGCAA
AAGGGCCTAGCTTGGACTCAGAATAATCCAGCCTTATCCCAACCATAAAA
TAAAAGCAGAATGGTAGCTGGATTGTAGCTGCTATTAGCAATATGAAACC
TCTTACATCAGTTACAATTTATATGCAGAAATATTTATATGCAGAAATAT
TGCTATTGCCTTAACCCAGAAATTATCACTGTTATTCTTTAGAATGGTGC
AAAGAGGCATGATACATTGTATCATTATTGCCCTGAAAGAAAGAGATTAG
GGAAAGTATTAGAAATAAGATAAACAAAAAAGTATATTAAAAGAAGAAAG
CATTTTTTAAAATTACAAATGCAAAATTACCCTGATTTGGTCAATATGTG
TACCCTGTTACTTCTCCCCTTCCTATGACATGAACTTAACCATAGAAAAG
AAGGGGAAAGAAAACATCAAGGGTCCCATAGACTCACCCTGAAGTTCTCA
GGATCCACGTGCAGCTTGTCACAGTGCAGCTCACTCAGCTGGGCAAAGGT
GCCCTTGAGGTTGTCCAGGTGAGCCAGGCCATCACTAAAGGCACCGAGCA
CTTTCTTGCCATGAGCCTTCACCTTAGGGTTGCCCATAACAGCATCAGGA
GTGGACAGATCCCCAAAGGACTCAAAGAACCTCTGGGTCCAAGGGTAGAC
CACCAGCAGCCTAAGGGTGGGAAAATAGACCAATAGGCAGAGAGAGTCAG
TGCCTATCAGAAACCCAAGAGTCTTCTCTGTCTCCACATGCCCAGTTTCT
ATTGGTCTCCTTAAACCTGTCTTGTAACCTTGATACCAACCTGCCCAGGG
CCTCACCACCAACGGCATCCACGTTCACCTTGCCCCACAGGGCAGTAACG
GCAGACTTCTCCTCAGGAGTCAGGTGCACCATGGTGTCTGTTTGAGGTTG
CTAGTGAACACAGTTGTGTCAGAAGCAAATGT

In certain embodiments, the human β^{A}-globin gene encoding a glutamic acid to alanine mutation at codon 22 and a threonine to glutamine mutation at codon 87 comprises the nucleotide sequence set forth in SEQ ID NO: 69, which is provided below:

[SEQ ID NO: 69]
gcaatgaaaataaatgttttttattaggcagaatccagatgctcaaggccc
ttcataatatcccccagtttagtagttggacttagggaacaaaggaacctt
taatagaaattggacagcaagaaagcgagcttagtgatacttgtgggccag
ggcattagccacaccagccaccactttctgataggcagcctgcactggtgg
ggtgAATTCTTTGCCAAAGTGATGGGCCAGCACACAGACCAGCACGTTGCC
CAGGAGCTGTGGGAGGAAGATAAGAGGTATGAACATGATTAGCAAAAGGGC
CTAGCTTGGACTCAGAATAATCCAGCCTTATCCCAACCATAAAATAAAAGC
AGAATGGTAGCTGGATTGTAGCTGCTATTAGCAATATGAAACCTCTTACAT
CAGTTACAATTTATATGCAGAAATATTTATATGCAGAAATATTGCTATTGC
CTTAACCCAGAAATTATCACTGTTATTCTTTAGAATGGTGCAAAGAGGCAT
GATACATTGTATCATTATTGCCCTGAAAGAAAGAGATTAGGGAAAGTATTA
GAAATAAGATAAACAAAAAAGTATATTAAAAGAAGAAAGCATTTTTTAAAA
TTACAAATGCAAAATTACCCTGATTTGGTCAATATGTGTACCCTGTTACTT
CTCCCCTTCCTATGACATGAACTTAACCATAGAAAAGAAGGGGAAAGAAAA
CATCAAGGGTCCCATAGACTCACCCTGAAGTTCTCAGGATCCACGTGCAGC
TTGTCACAGTGCAGCTCACTCAGTTGGGCAAAGGTGCCCTTGAGGTTGTCC
AGGTGAGCCAGGCCATCACTAAAGGCACCGAGCACTTTCTTGCCATGAGCC
TTCACCTTAGGGTTGCCCATAACAGCATCAGGAGTGGACAGATCCCCAAAG
GACTCAAAGAACCTCTGGGTCCAAGGGTAGACCACCAGCAGCCTAAGGGTG
GGAAAATAGACCAATAGGCAGAGAGAGTCAGTGCCTATCAGAAACCCAAGA
GTCTTCTCTGTCTCCACATGCCCAGTTTCTATTGGTCTCCTTAAACCTGTC
TTGTAACCTTGATACCAACCTGCCCAGGGCCTCACCACCAACGGCATCCAC
GTTCACCTTGCCCCACAGGGCAGTAACGGCAGACTTCTCCTCAGGAGTCAG
GTGCACCATGGTGTCTGTTTGAGGTTGCTAGTGAACACAGTTGTGTCAGAA
GCAAATGT

In certain embodiments, the human β^{A}-globin gene encoding a glutamic acid to alanine mutation at codon 22 and a threonine to glutamine mutation at codon 87 comprises the nucleotide sequence set forth in SEQ ID NO: 70, which is provided below:

[SEQ ID NO: 70]
gcaatgaaaataaatgttttttattaggcagaatccagatgctcaaggccc
ttcataatatcccccagtttagtagttggacttagggaacaaaggaacctt
taatagaaattggacagcaagaaagcgagcttagtgatacttgtgggccag
ggcattagccacaccagccaccactttctgataggcagcctgcactggtgg
ggtgAATTCTTTGCCAAAGTGATGGGCCAGCACACAGACCAGCACGTTGCC
CAGGAGCTGTGGGAGGAAGATAAGAGGTATGAACATGATTAGCAAAAGGGC
CTAGCTTGGACTCAGAATAATCCAGCCTTATCCCAACCATAAAATAAAAGC
AGAATGGTAGCTGGATTGTAGCTGCTATTAGCAATATGAAACCTCTTACAT
CAGTTACAATTTATATGCAGAAATATTTATATGCAGAAATATTGCTATTGC
CTTAACCCAGAAATTATCACTGTTATTCTTTAGAATGGTGCAAAGAGGCAT
GATACATTGTATCATTATTGCCCTGAAAGAAAGAGATTAGGGAAAGTATTA
GAAATAAGATAAACAAAAAAGTATATTAAAAGAAGAAAGCATTTTTTAAAA
TTACAAATGCAAAATTACCCTGATTTGGTCAATATGTGTACCCTGTTACTT
CTCCCCTTCCTATGACATGAACTTAACCATAGAAAAGAAGGGGAAAGAAAA
CATCAAGGGTCCCATAGACTCACCCTGAAGTTCTCAGGATCCACGTGCAGC
TTGTCACAGTGCAGCTCACTCAGCTGGGCAAAGGTGCCCTTGAGGTTGTCC
AGGTGAGCCAGGCCATCACTAAAGGCACCGAGCACTTTCTTGCCATGAGCC
TTCACCTTAGGGTTGCCCATAACAGCATCAGGAGTGGACAGATCCCCAAAG
GACTCAAAGAACCTCTGGGTCCAAGGGTAGACCACCAGCAGCCTAAGGGTG
GGAAAATAGACCAATAGGCAGAGAGAGTCAGTGCCTATCAGAAACCCAAGA
GTCTTCTCTGTCTCCACATGCCCAGTTTCTATTGGTCTCCTTAAACCTGTC
TTGTAACCTTGATACCAACCTGCCCAGGGCCTCACCACCAACTGCATCCAC
GTTCACCTTGCCCCACAGGGCAGTAACGGCAGACTTCTCCTCAGGAGTCAG
GTGCACCATGGTGTCTGTTTGAGGTTGCTAGTGAACACAGTTGTGTCAGAA
GCAAATGT

In certain embodiments, the human β^{A}-globin gene encoding a glutamic acid to alanine mutation at codon 22 and a threonine to glutamine mutation at codon 87 comprises the nucleotide sequence set forth in SEQ ID NO: 71, which is provided below:

[SEQ ID NO: 71]
gcaatgaaaataaatgttttttattaggcagaatccagatgctcaagg
cccttcataatatcccccagtttagtagttggacttagggaacaaagg
aacctttaatagaaattggacagcaagaaagcgagcttagtgatactt
gtgggccagggcattagccacaccagccaccactttctgataggcagc
ctgcactggtggggtgAATTCTTTGCCAAAGTGATGGGCCAGCACACA
GACCAGCACGTTGCCCAGGAGCTGTGGGAGGAAGATAAGAGGTATGAA
CATGATTAGCAAAAGGGCCTAGCTTGGACTCAGAATAATCCAGCCTTA
TCCCAACCATAAAATAAAAGCAGAATGGTAGCTGGATTGTAGCTGCTA
TTAGCAATATGAAACCTCTTACATCAGTTACAATTTATATGCAGAAAT
ATTTATATGCAGAAATATTGCTATTGCCTTAACCCAGAAATTATCACT
GTTATTCTTTAGAATGGTGCAAAGAGGCATGATACATTGTATCATTAT
TGCCCTGAAAGAAAGAGATTAGGGAAAGTATTAGAAATAAGATAAACA
AAAAAGTATATTAAAAGAAGAAAGCATTTTTTAAAATTACAAATGCAA
AATTACCCTGATTTGGTCAATATGTGTACCCTGTTACTTCTCCCCTTC
CTATGACATGAACTTAACCATAGAAAAGAAGGGGAAAGAAAACATCAA
GGGTCCCATAGACTCACCCTGAAGTTCTCAGGATCCACGTGCAGCTTG
TCACAGTGCAGCTCACTCAGCTGGGCAAAGGTGCCCTTGAGGTTGTCC
AGGTGAGCCAGGCCATCACTAAAGGCACCGAGCACTTTCTTGCCATGA
GCCTTCACCTTAGGGTTGCCCATAACAGCATCAGGAGTGGACAGATCC
CCAAAGGACTCAAAGAACCTCTGGGTCCAAGGGTAGACCACCAGCAGC
CTAAGGGTGGGAAAATAGACCAATAGGCAGAGAGAGTCAGTGCCTATC
AGAAACCCAAGAGTCTTCTCTGTCTCCACATGCCCAGTTTCTATTGGT
CTCCTTAAACCTGTCTTGTAACCTTGATACCAACCTGCCCAGGGCCTC
ACCACCAACAGCATCCACGTTCACCTTGCCCCACAGGGCAGTAACGGC
AGACTTCTCCTCAGGAGTCAGGTGCACCATGGTGTCTGTTTGAGGTTG
CTAGTGAACACAGTTGTGTCAGAAGCAAATGT

In certain embodiments, the human β^{A}-globin gene encoding a glutamic acid to alanine mutation at codon 22 and a threonine to glutamine mutation at codon 87 comprises the nucleotide sequence set forth in SEQ ID NO: 72, which is provided below:

[SEQ ID NO: 72]
gcaatgaaaataaatgttttttattaggcagaatccagatgctcaagg
cccttcataatatcccccagtttagtagttggacttagggaacaaagg
aacctttaatagaaattggacagcaagaaagcgagcttagtgatactt
gtgggccagggcattagccacaccagccaccactttctgataggcagc
ctgcactggtggggtgAATTCTTTGCCAAAGTGATGGGCCAGCACACA
GACCAGCACGTTGCCCAGGAGCTGTGGGAGGAAGATAAGAGGTATGAA
CATGATTAGCAAAAGGGCCTAGCTTGGACTCAGAATAATCCAGCCTTA
TCCCAACCATAAAATAAAAGCAGAATGGTAGCTGGATTGTAGCTGCTA
TTAGCAATATGAAACCTCTTACATCAGTTACAATTTATATGCAGAAAT
ATTTATATGCAGAAATATTGCTATTGCCTTAACCCAGAAATTATCACT
GTTATTCTTTAGAATGGTGCAAAGAGGCATGATACATTGTATCATTAT
TGCCCTGAAAGAAAGAGATTAGGGAAAGTATTAGAAATAAGATAAACA
AAAAAGTATATTAAAAGAAGAAAGCATTTTTTAAAATTACAAATGCAA
AATTACCCTGATTTGGTCAATATGTGTACCCTGTTACTTCTCCCCTTC
CTATGACATGAACTTAACCATAGAAAAGAAGGGGAAAGAAAACATCAA
GGGTCCCATAGACTCACCCTGAAGTTCTCAGGATCCACGTGCAGCTTG
TCACAGTGCAGCTCACTCAGCTGGGCAAAGGTGCCCTTGAGGTTGTCC
AGGTGAGCCAGGCCATCACTAAAGGCACCGAGCACTTTCTTGCCATGA
GCCTTCACCTTAGGGTTGCCCATAACAGCATCAGGAGTGGACAGATCC
CCAAAGGACTCAAAGAACCTCTGGGTCCAAGGGTAGACCACCAGCAGC
CTAAGGGTGGGAAAATAGACCAATAGGCAGAGAGAGTCAGTGCCTATC
AGAAACCCAAGAGTCTTCTCTGTCTCCACATGCCCAGTTTCTATTGGT
CTCCTTAAACCTGTCTTGTAACCTTGATACCAACCTGCCCAGGGCCTC
ACCACCAACCGCATCCACGTTCACCTTGCCCCACAGGGCAGTAACGGC
AGACTTCTCCTCAGGAGTCAGGTGCACCATGGTGTCTGTTTGAGGTTG
CTAGTGAACACAGTTGTGTCAGAAGCAAATGT

In certain embodiments, the human β^{A}-globin gene encoding a glutamic acid to alanine mutation at codon 22 and a threonine to glutamine mutation at codon 87 comprises the nucleotide sequence set forth in SEQ ID NO: 73, which is provided below:

[SEQ ID NO: 73]
gcaatgaaaataaatgttttttattaggcagaatccagatgctcaagg
cccttcataatatcccccagtttagtagttggacttagggaacaaagg
aacctttaatagaaattggacagcaagaaagcgagcttagtgatactt
gtgggccagggcattaGCCACACCAGCCACCACTTTCTGATAGGCAGC
CTGCACTGGTGGGGTGAATTCTTTGCCAAAGTGATGGGCCAGCACACA
GACCAGCACGTTGCCCAGGAGCTGTGGGAGGAAGATAAGAGGTATGAA
CATGATTAGCAAAAGGGCCTAGCTTGGACTCAGAATAATCCAGCCTTA
TCCCAACCATAAAATAAAAGCAGAATGGTAGCTGGATTGTAGCTGCTA
TTAGCAATATGAAACCTCTTACATCAGTTACAATTTATATGCAGAAAT
ATTTATATGCAGAAATATTGCTATTGCCTTAACCCAGAAATTATCACT
GTTATTCTTTAGAATGGTGCAAAGAGGCATGATACATTGTATCATTAT
TGCCCTGAAAGAAAGAGATTAGGGAAAGTATTAGAAATAAGATAAACA
AAAAAGTATATTAAAAGAAGAAAGCATTTTTTAAAATTACAAATGCAA
AATTACCCTGATTTGGTCAATATGTGTACCCTGTTACTTCTCCCCTTC
CTATGACATGAACTTAACCATAGAAAAGAAGGGGAAAGAAAACATCAA
GGGTCCCATAGACTCACCCTGAAGTTCTCAGGATCCACGTGCAGCTTG
TCACAGTGCAGCTCACTCAGTTGGGCAAAGGTGCCCTTGAGGTTGTCC
AGGTGAGCCAGGCCATCACTAAAGGCACCGAGCACTTTCTTGCCATGA
GCCTTCACCTTAGGGTTGCCCATAACAGCATCAGGAGTGGACAGATCC
CCAAAGGACTCAAAGAACCTCTGGGTCCAAGGGTAGACCACCAGCAGC
CTAAGGGTGGGAAAATAGACCAATAGGCAGAGAGAGTCAGTGCCTATC
AGAAACCCAAGAGTCTTCTCTGTCTCCACATGCCCAGTTTCTATTGGT
CTCCTTAAACCTGTCTTGTAACCTTGATACCAACCTGCCCAGGGCCTC
ACCACCAACTGCATCCACGTTCACCTTGCCCCACAGGGCAGTAACGGC
AGACTTCTCCTCAGGAGTCAGGTGCACCATGGTGTCTGTTTGAGGTTG
CTAGTGAACACAGTTGTGTCAGAAGCAAATGT

In certain embodiments, the human β^{A}-globin gene encoding a glutamic acid to alanine mutation at codon 22 and a threonine to glutamine mutation at codon 87 comprises the nucleotide sequence set forth in SEQ ID NO: 74, which is provided below:

[SEQ ID NO: 74]
gcaatgaaaataaatgttttttattaggcagaatccagatgctcaagg
cccttcataatatcccccagtttagtagttggACTTAGGGAACAAAGG
AACCTTTAATAGAAATTGGACAGCAAGAAAGCGAGCTTAGTGATACTT
GTGGGCCAGGGCATTAGCCACACCAGCCACCACTTTCTGATAGGCAGC
CTGCACTGGTGGGGTGAATTCTTTGCCAAAGTGATGGGCCAGCACACA
GACCAGCACGTTGCCCAGGAGCTGTGGGAGGAAGATAAGAGGTATGAA
CATGATTAGCAAAAGGGCCTAGCTTGGACTCAGAATAATCCAGCCTTA
TCCCAACCATAAAATAAAAGCAGAATGGTAGCTGGATTGTAGCTGCTA
TTAGCAATATGAAACCTCTTACATCAGTTACAATTTATATGCAGAAAT
ATTTATATGCAGAAATATTGCTATTGCCTTAACCCAGAAATTATCACT
GTTATTCTTTAGAATGGTGCAAAGAGGCATGATACATTGTATCATTAT
TGCCCTGAAAGAAAGAGATTAGGGAAAGTATTAGAAATAAGATAAACA
AAAAAGTATATTAAAAGAAGAAAGCATTTTTTAAAATTACAAATGCAA
AATTACCCTGATTTGGTCAATATGTGTACCCTGTTACTTCTCCCCTTC
CTATGACATGAACTTAACCATAGAAAAGAAGGGGAAAGAAAACATCAA
GGGTCCCATAGACTCACCCTGAAGTTCTCAGGATCCACGTGCAGCTTG
TCACAGTGCAGCTCACTCAGTTGGGCAAAGGTGCCCTTGAGGTTGTCC
AGGTGAGCCAGGCCATCACTAAAGGCACCGAGCACTTTCTTGCCATGA
GCCTTCACCTTAGGGTTGCCCATAACAGCATCAGGAGTGGACAGATCC
CCAAAGGACTCAAAGAACCTCTGGGTCCAAGGGTAGACCACCAGCAGC
CTAAGGGTGGGAAAATAGACCAATAGGCAGAGAGAGTCAGTGCCTATC
AGAAACCCAAGAGTCTTCTCTGTCTCCACATGCCCAGTTTCTATTGGT
CTCCTTAAACCTGTCTTGTAACCTTGATACCAACCTGCCCAGGGCCTC
ACCACCAACAGCATCCACGTTCACCTTGCCCCACAGGGCAGTAACGGC
AGACTTCTCCTCAGGAGTCAGGTGCACCATGGTGTCTGTTTGAGGTTG
CTAGTGAACACAGTTGTGTCAGAAGCAAATGT

In certain embodiments, the human β^{A}-globin gene encoding a glutamic acid to alanine mutation at codon 22 and a threonine to glutamine mutation at codon 87 comprises the nucleotide sequence set forth in SEQ ID NO: 75, which is provided below:

[SEQ ID NO: 75]
gcaatgaaaataaatgttttttattaggcagaatccagatgctcaagg
cccttcataatatcccccagtttagtagttggACTTAGGGAACAAAGG
AACCTTTAATAGAAATTGGACAGCAAGAAAGCGAGCTTAGTGATACTT
GTGGGCCAGGGCATTAGCCACACCAGCCACCACTTTCTGATAGGCAGC
CTGCACTGGTGGGGTGAATTCTTTGCCAAAGTGATGGGCCAGCACACA
GACCAGCACGTTGCCCAGGAGCTGTGGGAGGAAGATAAGAGGTATGAA
CATGATTAGCAAAAGGGCCTAGCTTGGACTCAGAATAATCCAGCCTTA
TCCCAACCATAAAATAAAAGCAGAATGGTAGCTGGATTGTAGCTGCTA
TTAGCAATATGAAACCTCTTACATCAGTTACAATTTATATGCAGAAAT
ATTTATATGCAGAAATATTGCTATTGCCTTAACCCAGAAATTATCACT
GTTATTCTTTAGAATGGTGCAAAGAGGCATGATACATTGTATCATTAT
TGCCCTGAAAGAAAGAGATTAGGGAAAGTATTAGAAATAAGATAAACA
AAAAAGTATATTAAAAGAAGAAAGCATTTTTTAAAATTACAAATGCAA
AATTACCCTGATTTGGTCAATATGTGTACCCTGTTACTTCTCCCCTTC
CTATGACATGAACTTAACCATAGAAAAGAAGGGGAAAGAAAACATCAA
GGGTCCCATAGACTCACCCTGAAGTTCTCAGGATCCACGTGCAGCTTG
TCACAGTGCAGCTCACTCAGTTGGGCAAAGGTGCCCTTGAGGTTGTCC
AGGTGAGCCAGGCCATCACTAAAGGCACCGAGCACTTTCTTGCCATGA
GCCTTCACCTTAGGGTTGCCCATAACAGCATCAGGAGTGGACAGATCC
CCAAAGGACTCAAAGAACCTCTGGGTCCAAGGGTAGACCACCAGCAGC
CTAAGGGTGGGAAAATAGACCAATAGGCAGAGAGAGTCAGTGCCTATC
AGAAACCCAAGAGTCTTCTCTGTCTCCACATGCCCAGTTTCTATTGGT
CTCCTTAAACCTGTCTTGTAACCTTGATACCAACCTGCCCAGGGCCTC
ACCACCAACCGCATCCACGTTCACCTTGCCCCACAGGGCAGTAACGGC
AGACTTCTCCTCAGGAGTCAGGTGCACCATGGTGTCTGTTTGAGGTTG
CTAGTGAACACAGTTGTGTCAGAAGCAAATGT

In certain embodiments, the site of the human β-globin gene IVS2 deletion is located at nucleotides 650-652 of SEQ ID NO: 36. In certain embodiments, nucleotides 1 to 261 of SEQ ID NO: 36 is the nucleotide sequence of exon 3 of the human β-globin gene. In certain embodiments, nucleotides 738 to 960 of SEQ ID NO: 36 is the nucleotide sequence of exon 2 of the human β-globin gene. certain embodiments, nucleotides 1091 to 1232 of SEQ ID NO: 36 is the nucleotide sequence of exon 1 of the human β-globin gene.

### 3.3. Promoters

In accordance with the presently disclosed subject matter, the expression cassette can further comprise a β-globin promoter. In certain embodiments, the β-globin promoter is positioned between the globin gene or functional portion thereof and the β-globin LCR. The length and the sequence of the β-globin promoter can vary. In certain embodiments, the β-globin promoter is from about 100 bp to about 1600 bp in length, e.g., from about 200 bp to about 700 bp, from about 100 bp to about 200 bp, from about 200 bp to about 300 bp, from about 300 bp to about 400 bp, from about 400 bp to about 500 bp, from about 500 bp to about 600 bp, from about 600 bp to about 700 bp, from about 700 bp to about 800 bp, from about 800 bp to about 900 bp, from about 900 bp to about 1000 bp, from about 1000 bp to about 1100 bp, from about 1100 bp to about 1200 bp, from about 1200 bp to about 1300 bp, from about 1300 bp to about 1400 bp, from about 1400 bp to about 1500 bp, or from about 1500 bp to about 1600 bp in length. In certain embodiments, the β-globin promoter a human β-globin promoter that is about 130 bp, about 613 bp, about 265 bp, or about 1555 bp, in length. In certain embodiments, the β-globin promoter is a human β-globin promoter that is about 613 bp in length. In certain non-limiting embodiments, the human β-globin promoter has the nucleotide sequence set forth in SEQ ID NO:10, which is provided below:

[SEQ ID NO: 10]
AAGCAATAGATGGCTCTGCCCTGACTTTTATGCCCAGCCCTGGCTCCT
GCCCTCCCTGCTCCTGGGAGTAGATTGGCCAACCCTAGGGTGTGGCTC
CACAGGGTGAGGTCTAAGTGATGACAGCCGTACCTGTCCTTGGCTCTT
CTGGCACTGGCTTAGGAGTTGGACTTCAAACCCTCAGCCCTCCCTCTA
AGATATATCTCTTGGCCCCATACCATCAGTACAAATTGCTACTAAAAA
CATCCTCCTTTGCAAGTGTATTTACGTAATATTTGGAATCACAGCTTG
GTAAGCATATTGAAGATCGTTTTCCCAATTTTCTTATTACACAAATAA
GAAATTGATGCACTAAAAGTGGAAGAGTTTTGTCTACCATAATTCAGC
TTTGGGATATGTAGATGGATCTCTTCCTGCGTCTCCAGAATATGCAAA
ATACTTACAGGACAGAATGGATGAAAACTCTACCTCAGTTCTAAGCAT
ATCTTCTCCTTATTTGGATTAAAACCTTCTGGTAAGAAAAGAAAAAAA
ATATATATATATATGTGTATATATACACACATACATATACATATATAT
GCATTCATTTGTTGTTGTTTTTCTTAATTTGCTCATG

In certain embodiments, the β-globin promoter is a human β-globin promoter that is about 265 bp in length. In certain non-limiting embodiments, the human β-globin promoter has the nucleotide sequence set forth in SEQ ID NO:11.

[SEQ ID NO: 11]
AAGCAATAGATGGCTCTGCCCTGACTTTTATGCCCAGCCCTGGCTCCT
GCCCTCCCTGCTCCTGGGAGTAGATTGGCCAACCCTAGGGTGTGGCTC
CACAGGGTGAGGTCTAAGTGATGACAGCCGTACCTGTCCTTGGCTCTT
CTGGCACTGGCTTAGGAGTTGGACTTCAAACCCTCAGCCCTCCCTCTA
AGATATATCTCTTGGCCCCATACCATCAGTACAAATTGCTACTAAAAA
CATCCTCCTTTGCAAGTGTATTTAC

### 3.4. Human β-Globin 3′ Enhancers

Additionally or alternatively, a presently disclosed expression cassette can further comprise a human β-globin 3′ enhancer. In certain embodiments, the human β-globin 3′ enhancer is positioned in the upstream of the globin gene or functional portion thereof. In certain embodiments, the β-globin 3′ enhancer is from about 500 bp to about 1000 bp in length, e.g., from about 500 bp to about 600 bp, from about 600 bp to about 700 bp, from about 700 bp to about 800 bp, or from about 800 bp to about 900 bp in length. In certain embodiments, the human β-globin 3′ enhancer is about 879 bp in length.

In certain embodiments, the human β-globin 3′ enhancer has the nucleotide sequence set forth in SEQ ID NO:12, which is provided below.

[SEQ ID NO: 12]
TAGGTATTGAATAAGAAAAATGAAGTTAAGGTGGTTGATGGTAACACT
ATGCTAATAACTGCAGAGCCAGAAGCACCATAAGGGACATGATAAGGG
AGCCAGCAGACCTCTGATCTCTTCCTGAATGCTAATCTTAAACATCCT
GAGGAAGAATGGGACTTCCATTTGGGGTGGGCCTATGATAGGGTAATA
AGACAGTAGTGAATATCAAGCTACAAAAAGCCCCCTTTCAAATTCTTC
TCAGTCCTAACTTTTCATACTAAGCCCAGTCCTTCCAAAGCAGACTGT
GAAAGAGTGATAGTTCCGGGAGACTAGCACTGCAGATTCCGGGTCACT
GTGAGTGGGGGAGGCAGGGAAGAAGGGCTCACAGGACAGTCAAACCAT
GCCCCCTGTTTTTCCTTCTTCAAGTAGACCTCTATAAGACAACAGAGA
CAACTAAGGCTGAGTGGCCAGGCGAGGAGAAACCATCTCGCCGTAAAA
CATGGAAGGAACACTTCAGGGGAAAGGTGGTATCTCTAAGCAAGAGAA
CTGAGTGGAGTCAAGGCTGAGAGATGCAGGATAAGCAAATGGGTAGTG
AAAAGACATTCATGAGGACAGCTAAAACAATAAGTAATGTAAAATACA
GCATAGCAAAACTTTAACCTCCAAATCAAGCCTCTACTTGAATCCTTT
TCTGAGGGATGAATAAGGCATAGGCATCAGGGGCTGTTGCCAATGTGC
ATTAGCTGTTTGCAGCCTCACCTTCTTTCATGGAGTTTAAGATATAGT
GTATTTTCCCAAGGTTTGAACTAGCTCTTCATTTCTTTATGTTTTAAA
TGCACTGACCTCCCACATTCCCTTTTTAGTAAAATATTCAGAAATAAT
TTAAATACATCATTG

In certain embodiments, the human β-globin 3′ enhancer has the nucleotide sequence set forth in SEQ ID NO: 78, which is provided below.

[SEQ ID NO: 78]
CTAGGTATTGA ATAAGAAAAA TGAAGTTAAG GTGGTTGATG
GTAACACTAT GCTAATAACT GCAGAGCCAG AAGCACCATA
AGGGACATGA TAAGGGAGCC AGCAGACCTC TGATCTCTTC
CTGAATGCTA ATCTTAAACA TCCTGAGGAA GAATGGGACT
TCCATTTGGG GTGGGCCTAT GATAGGGTAA TAAGACAGTA
GTGAATATCA AGCTACAAAA AGCCCCCTTT CAAATTCTTC
TCAGTCCTAA CTTTTCATAC TAAGCCCAGT CCTTCCAAAG
CAGACTGTGA AAGAGTGATA GTTCCGGGAG ACTAGCACTG
CAGATTCCGG GTCACTGTGA GTGGGGGAGG CAGGGAAGAA
GGGCTCACAG GACAGTCAAA CCATGCCCCC TGTTTTTCCT
TCTTCAAGTA GACCTCTATA AGACAACAGA GACAACTAAG
GCTGAGTGGC CAGGCGAGGA GAAACCATCT CGCCGTAAAA
CATGGAAGGA ACACTTCAGG GGAAAGGTGG TATCTCTAAG
CAAGAGAACT GAGTGGAGTC AAGGCTGAGA GATGCAGGAT
AAGCAAATGG GTAGTGAAAA GACATTCATG AGGACAGCTA
AAACAATAAG TAATGTAAAA TACAGCATAG CAAAACTTTA
ACCTCCAAAT CAAGCCTCTA CTTGAATCCT TTTCTGAGGG
ATGAATAAGG CATAGGCATC AGGGGCTGTT GCCAATGTGC
ATTAGCTGTT TGCAGCCTCA CCTTCTTTCA TGGAGTTTAA
GATATAGTGT ATTTTCCCAA GGTTTGAACT AGCTCTTCAT
TTCTTTATGT TTTAAATGCA CTGACCTCCC ACATTCCCTT
TTTAGTAAAA TATTCAGAAA TAATTTAAAT ACATCATT

### 3.5. Erythroid-Specific Enhancers

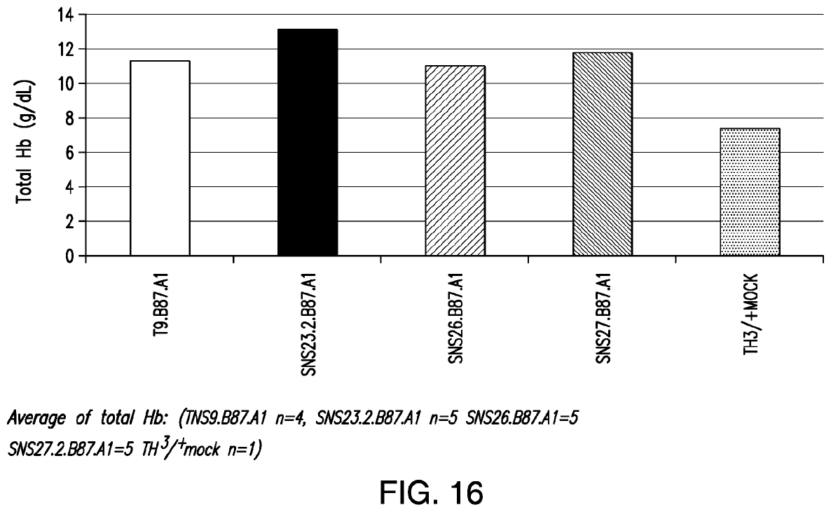

Furthermore, a presently disclosed expression cassette can further comprise at least one erythroid-specific enhancer. The presently disclosed expression cassette allows for expression of a globin gene (e.g., human β-globin gene) in erythroid-specific fashion. The erythroid-specific enhancer can enhance the expression of the globin gene in erythroid-specific fashion. For example, the erythroid-specific enhancer lack enhancer activity in non-erythroid tissues. In particularly, for the β-globin LCR that lacks a HS2 region, which primarily functions as an expression enhancer, the addition of one or more erythroid-specific enhancers can compensate the enhancing activity of a HS2 region. Additionally, for an expression cassette that lacks a β-globin LCR (e.g., SNS26.B87.A1) or the β-globin LCR consists essentially of a HS3 region and does not comprise a HS1 region, a HS2 region, or a HS4 region (e.g., SN27.2B87.A1), the addition of one or more erythroid-specific enhancers are capable of driving the expression of the β-globin gene. See e.g., Table 1 and FIG. 16. Furthermore, the presently disclosed erythroid-specific enhancers do not decrease or reduce the titer of a vector comprising the expression cassette. The length of the erythroid-specific enhancer can vary, e.g., from about 100 bp to about 200 bp, from about 100 bp to about 120 bp, from about 120 bp to about 140 bp, from about 140 bp to about 200 (e.g., from about 140 bp to about 150 bp, from about 150 bp to about 160 bp, from about 160 bp to about 170 bp, from about 170 bp to about 180 bp, from about 180 bp to about 190 bp, or from about 190 bp to about 200 bp). In certain embodiments, the erythroid-specific enhancer has a length of from about 140 bp to about 200 bp. In certain non-limiting embodiments, the erythroid-specific enhancer has a length of 152 bp, which has the nucleotide sequence set forth in SEQ ID NO:13, which is provided below:

[SEQ ID NO: 13]
TCTCCCACGCCCTGGTCTCAGCTTGGGGAGTGGTCAGACCCCAATGGC
GATAAACTCTGGCAACTTTATCTGTGcaCTGCAGGCTCAGCCCCAAca
GCTTTAGCTTTCACAAGCAGGCAGGGGAAGGGAAACACATATCTCCAG
ATATGAGG

In certain non-limiting embodiments, the erythroid-specific enhancer has a length of 157 bp, which has the nucleotide sequence set forth in SEQ ID NO:14, which is provided below:

[SEQ ID NO: 14]
CTAAACCCCTCCCCCACCCTAGCCCCAAGCTTCATCTTAGCTCCACTC
CTGACCCTATCCAGCTAAAGGTCCCCACCCAGCTCCTGCCTATCTAGT
CATTGCATATGGCAAGACTTGAAAGTCCTATCTCAAAGCAGCAGAATT
ATCAGCTACGACT

In certain non-limiting embodiments, the erythroid-specific enhancer has a length of 141 bp, which has the nucleotide sequence set forth in SEQ ID NO:15, which is provided below:

[SEQ ID NO: 15]
CCATCCCCCAGCACTCCCTGCCCCCACAGCCCAGACTTGACCAACTCC
CAGCTccGCCTGGGACTTCCAGATATGGGGCCCCACCCTTGCAGGCCT
TGGGGACGCTGAAGATATTGACTATCTGCGTGCCggAAAAGGGTG

In certain non-limiting embodiments, the erythroid-specific enhancer has a length of 171 bp, which has the nucleotide sequence set forth in SEQ ID NO: 16, which is provided below:

[SEQ ID NO: 16]
AAAGGCTGGGGGTGGGAGTAGCGGATTTGAAGCACTTGTTGGCCTACA
GAGGTGTGGCAAGCAGAGCACCTCAGAACTCAGGCGTACTGCCCGCCG
CCCGAGCCCTGCGAGGGCCGATAGCGAGGGTGTGGCCCTTATCTGCAC
CCAGCAGAGCGCCGGCGGGGTACGGTC

In certain non-limiting embodiments, the erythroid-specific enhancer has a length of 195 bp, which has the nucleotide sequence set forth in SEQ ID NO:17, which is provided below:

[SEQ ID NO: 17]
CAGTTGCCTCAGCTGAGTATGTCTTCTAAAGATAATGTCGATTGTGTA
TGGCTGATGGGATTCTAGGACCAAGCAAGAGGTTTTTTTTTTTCCCCC
ACATACTTAACGTTTCTATATTTCTATTTGAATTCGACTGGACAGTTC
CATTTGAATTATTTCTCTCTCTCTCTCTCTCTGACACATTTTATCTTG
CCA

The erythroid-specific enhancer can be located within the β-globin LCR, e.g., between any two of the HS1, HS2, HS3, and HS4 regions. In certain embodiments, the erythroid-specific enhancer is located between the HS1 and HS3 regions within the β-globin LCR.

Alternatively, the erythroid-specific enhancer can be located upstream or downstream of the insulator, e.g., where the expression cassette does not comprise a β-globin LCR. In certain embodiments, the erythroid-specific enhancer is located upstream of the erythroid-specific enhancer.

Furthermore, the erythroid-specific enhancer can be located upstream or downstream of any of the HS1, HS2, HS3, and HS4 regions. In certain embodiments, the erythroid-specific enhancer is located upstream of the HS3 region.

In certain embodiments, the β-globin LCR comprises or consists essentially of a HS1 region having the nucleotide sequence set forth in SEQ ID NO: 3; a HS3 region having the nucleotide sequence set forth in SEQ ID NO: 5; a HS4 region having nucleotides 115 to 868 of SEQ ID NO: 6; and an erythroid-specific enhancer having the nucleotide sequence set forth in SEQ ID NO: 15, wherein the erythroid-specific enhancer is positioned between the HS1 region and the HS3 region, e.g., the β-globin LCR of SNS24.B87.A1 shown in FIG. 15.

In certain non-limiting embodiments, the β-globin LCR comprises or consists essentially of a HS1 region having the nucleotide sequence set forth in SEQ ID NO: 3; a HS3 region having the nucleotide sequence set forth in SEQ ID NO: 34; and a HS4 region having the nucleotide sequence set forth in SEQ ID NO: 35; and an erythroid-specific enhancer having the nucleotide sequence set forth in SEQ ID NO: 15, wherein the erythroid-specific enhancer is positioned between the HS1 region and the HS3 region, e.g., as the β-globin LCR of SNS22.2.B87.A1 shown in FIG. 13.

Erythroid-specific enhancers can be identified and determined by any suitable methods known in the art. The erythroid-specific enhancers can be positioned at the 3′ UTR (downstream) or the 5′ UTR (downstream) of the β-globin LCR. In certain embodiments, the at least one erythroid-specific enhancer is positioned in the 5′ UTR of the β-globin LCR, e.g., the upstream of the HS3 region. The expression cassette can comprise one, two, three, four, or five erythroid-specific enhancers. In certain embodiments, the expression cassette comprises one erythroid-specific enhancer. In certain embodiments, the expression cassette comprises two erythroid-specific enhancers. In certain embodiments, the expression cassette comprises three erythroid-specific enhancers. In certain embodiments, the expression cassette comprises four erythroid-specific enhancers. In certain non-limiting embodiments, the expression cassette comprises five erythroid-specific enhancers.

### 3.6. Insulators

In accordance with the presently disclosed subject matter, the expression cassette comprises at least one of the above-described insulators. In certain embodiments, a presently disclosed expression cassette comprises at least one insulator comprising the CTCF binding site sequence set forth in SEQ ID NO:18, for example, but not limited to, an insulator comprising the nucleotide sequence set forth in SEQ ID NO: 1, SEQ ID NO: 24, SEQ ID NO: 25, SEQ ID NO: 27, SEQ ID NO: 28, SEQ ID NO: 29, SEQ ID NO: 30, SEQ ID NO: 31, or SEQ ID NO: 32 or a fragment thereof. In certain embodiments, a presently disclosed expression cassette comprises at least one insulator comprising or having the nucleotide sequence set forth in SEQ ID NO: 1. In various non-limiting embodiments, the insulator can be incorporated or inserted into one or both LTRs or elsewhere in the region of a presently disclosed expression cassette that integrates into the cellular genome. In certain embodiments, the insulator is positioned at the 3′ end of the expression cassette. In certain embodiments, the insulator is positioned at the 5′ end of the expression cassette. In certain embodiments, the expression cassette comprises two of the insulator disclosed herein, e.g., an insulator comprising the nucleotide sequence set forth in SEQ ID NO: 1, SEQ ID NO: 24, SEQ ID NO: 25, SEQ ID NO: 27, SEQ ID NO: 28, SEQ ID NO: 29, SEQ ID NO: 30, SEQ ID NO: 31, or SEQ ID NO: 32 or a fragment thereof, where one insulator is positioned at the 3′ end and the other insulator is positioned at the 5′ end of the expression cassette.

The presently disclosed insulators possess powerful enhancer blocking activity. In certain embodiments, the insulators possess barrier activity in addition to enhancer blocking activity. The presently disclosed insulators substantially decrease the risks of insertional mutagenesis and genotoxicity associated with viral vectors. Furthermore, when a presently disclosed insulator is incorporated into a vector, the insulator does not adversely effect vector titers of the vector. In certain embodiments, the insulators increase the in vivo expression of the globin gene or functional portion thereof.

### 3.7. Exemplary Expression Cassettes

For the purpose of illustration and not limitation, FIGS. 1-4, 13, 14, and 15 show recombinant vectors comprising exemplary expression cassettes in accordance with certain embodiments of the presently disclosed subject matter.

FIG. 1 shows a recombinant vector comprising a presently disclosed expression cassette that comprises a human β^{A}-T870 globin gene, which is operably linked to a β-globin LCR that comprises a 860 bp HS2 region (e.g., one having the nucleotide sequence set forth in SEQ ID NO:9), a 1301 bp HS3 region (e.g., one having the nucleotide sequence set forth in SEQ ID NO:5), and a 1065 bp HS4 region (e.g., one having the nucleotide sequence set forth in SEQ ID NO:7).

FIG. 2 shows one exemplary recombinant vector comprising an expression cassette in accordance with one embodiment of the presently disclosed subject matter. FIG. 2 shows a recombinant vector comprising a presently disclosed expression cassette that comprises a human BA-T870 globin gene, which is operably linked to a β-globin LCR that comprises a 1.1 kb HS1 region (e.g., one having the nucleotide sequence set forth in SEQ ID NO:2), a 1301 bp HS3 region (e.g., one having the nucleotide sequence set forth in SEQ ID NO:5), and a 1065 bp HS4 region (e.g., one having the nucleotide sequence set forth in SEQ ID NO:6).

FIG. 3 shows one exemplary recombinant vector comprising an expression cassette in accordance with one embodiment of the presently disclosed subject matter. FIG. 3 shows a recombinant vector comprising a presently disclosed expression cassette that comprises a human BA-T870 globin gene, which is operably linked to a β-globin LCR that comprises a 602 bp HS1 region (e.g., one having the nucleotide sequence set forth in SEQ ID NO:3), a 1301 bp HS3 region (e.g., one having the nucleotide sequence set forth in SEQ ID NO:5), and a 446 bp HS4 region (e.g., one having the nucleotide sequence set forth in SEQ ID NO:8).

FIG. 4 shows one exemplary recombinant vector comprising an expression cassette in accordance with one embodiment of the presently disclosed subject matter. FIG. 4 shows a recombinant vector comprising a presently disclosed expression cassette that comprises a human BA-T870 globin gene, which is operably linked to a β-globin LCR that comprises a 1301 bp HS3 region (e.g., one having the nucleotide sequence set forth in SEQ ID NO:5), and a 1065 bp HS4 region (e.g., one having the nucleotide sequence set forth in SEQ ID NO:6). The expression cassette shown in FIG. 4 also comprises the following five erythroid-specific enhancers (shown as “EE5” in FIG. 4): one erythroid-specific enhancer having the nucleotide sequence set forth in SEQ ID NO:13, one erythroid-specific enhancer having the nucleotide sequence set forth in SEQ ID NO: 14, one erythroid-specific enhancer having the nucleotide sequence set forth in SEQ ID NO: 15, one erythroid-specific enhancer having the nucleotide sequence set forth in SEQ ID NO:16, and one erythroid-specific enhancer having the nucleotide sequence set forth in SEQ ID NO:17.

FIG. 13 shows the following five exemplary recombinant vectors: SNS22.2.B87A1, which comprises an expression cassette that comprises a human β^{A}-T870 globin gene, which is operably linked to a β-globin LCR that comprises a 602 bp HS1 region (e.g., one having the nucleotide sequence set forth in SEQ ID NO:3), a 816 bp HS2 region (e.g., one having the nucleotide sequence set forth in SEQ ID NO:33), a 1301 bp HS3 region (e.g., one having the nucleotide sequence set forth in SEQ ID NO:34), and a 754 bp HS4 region (e.g., one having the nucleotide sequence set forth in SEQ ID NO:35);

SNS23.2.B87A1, which comprises an expression cassette that comprises a human β^{A-T87Q} globin gene, which is operably linked to a β-globin LCR that comprises a 816 bp HS2 region (e.g., one having the nucleotide sequence set forth in SEQ ID NO:33), a 1301 bp HS3 region (e.g., one having the nucleotide sequence set forth in SEQ ID NO:34), and a 754 bp HS4 region (e.g., one having the nucleotide sequence set forth in SEQ ID NO:35), wherein the β-globin LCR does not comprise a HS1 region;

SNS24.2.B87A1, which comprises an expression cassette that comprises a human β^{A-T87Q} globin gene, which is operably linked to a β-globin LCR that comprises a 602 bp HS1 region (e.g., one having the nucleotide sequence set forth in SEQ ID NO:3), a 1301 bp HS3 region (e.g., one having the nucleotide sequence set forth in SEQ ID NO:34), a 754 bp HS4 region (e.g., one having the nucleotide sequence set forth in SEQ ID NO:35); and an erythroid-specific enhancer having the nucleotide sequence set forth in SEQ ID NO:15, wherein the β-globin LCR does not comprise a HS2 region;

SNS26.B87A1, which comprises an expression cassette that comprises a human β^{A}-T870 globin gene, and an erythroid-specific enhancer having the nucleotide sequence set forth in SEQ ID NO:15, wherein the expression cassette does not comprise a β-globin LCR; and

SNS27.2.B87.A1, which comprises an expression cassette that comprises an insulator comprising the nucleotide sequence set forth in SEQ ID NO: 1, and a human β^{A}-T870 globin gene that is operably linked to a β-globin LCR comprising a 1301 bp HS3 region (e.g., one having the nucleotide sequence set forth in SEQ ID NO:34), and an erythroid-specific enhancer having the nucleotide sequence set forth in SEQ ID NO:15, wherein the β-globin LCR does not comprise a HS1 region, a HS2 region, or a HS4 region.

FIG. 15 shows five exemplary recombinant vectors including SNS22.B87A1, which comprises an expression cassette that comprises a human BA-1870 globin gene, which is operably linked to a β-globin LCR that comprises a 602 bp HS1 region (e.g., one having the nucleotide sequence set forth in SEQ ID NO:3), a 816 bp HS2 region (e.g., one having nucleotides 45 to 860 of SEQ ID NO: 9), a 1301 bp HS3 region (e.g., one having the nucleotide sequence set forth in SEQ ID NO:5), and a 754 bp HS4 region (e.g., one having nucleotides 115 to 868 of SEQ ID NO: 6);

SNS23.B87A1, which comprises an expression cassette that comprises a human BA-1870 globin gene, which is operably linked to a β-globin LCR that comprises a 816 bp HS2 region (e.g., one having nucleotides 45 to 860 of SEQ ID NO: 9), a 1301 bp HS3 region (e.g., one having the nucleotide sequence set forth in SEQ ID NO:5), and a 754 bp HS4 region (e.g., one having nucleotides 115 to 868 of SEQ ID NO: 6); SNS24.B87A1, which comprises an expression cassette that comprises a human β^{A}-T870 globin gene, which is operably linked to a β-globin LCR that comprises a 602 bp HS1 region (e.g., one having the nucleotide sequence set forth in SEQ ID NO:3), a 1301 bp HS3 region (e.g., one having the nucleotide sequence set forth in SEQ ID NO:5), a 754 bp HS4 region (e.g., one having nucleotides 115 to 868 of SEQ ID NO: 6); and an erythroid-specific enhancer having the nucleotide sequence set forth in SEQ ID NO:15; and

SNS26.B87.A1, which is also disclosed in FIG. 13 as described above.

As shown in FIGS. 1-4, 13, and 15, each of the expression cassettes comprises an insulator, wherein the insulator has the nucleotide sequence set forth in SEQ ID NO:1 (i.e., insulator A1). In addition, as shown in FIGS. 1-4, 13, 15, each of the expression cassettes comprises a human β-globin 3′ enhancer that is positioned upstream of the human β-globin gene, wherein the human β-globin 3′ enhancer has a length of 879 bp and has the nucleotide sequence set forth in SEQ ID NO: 12.

Additionally, as shown in FIGS. 13 and 15, each of SNS22.B87.A1, SNS23.B87.A1, SNS24.B87.A1, SNS22.2.B87.A1, SNS23.2.B87.A1, SNS24.2.B87.A1, SNS26.B87.A1, and SNS27.2B87.A1 comprises a 265 bp human β-globin promoter, wherein the human β-globin promoter has the nucleotide sequence set forth in SEQ ID NO: 11. TNS9.B87.A1 shown in FIG. 15 comprises a 613 bp human β-globin promoter, wherein the human β-globin promoter has the nucleotide sequence set forth in SEQ ID NO: 10.

### III. Vectors, Nucleases and CRISPR-Cas Systems

The presently disclosed subject matter provides vectors and delivery systems (e.g., a non-naturally occurring or engineered nucleases or a CRISPR-Cas system) comprising the above-described expression cassettes. The vectors and delivery systems are suitable delivery vehicles for the stable introduction of globin gene (e.g., human β-globin) into the genome of a broad range of target cells to increase expression of the globin protein (human β-globin protein) in the cell.

In certain embodiments, the vector is a retroviral vector (e.g., gamma retroviral vector or a lentiviral vector) that is employed for the introduction or transduction of the above-described expression cassette into the genome of a host cell (e.g., a hematopoietic stem cell, an embryonic stem cell, an induced pluripotent stem cell, or a hemogenic endothelium cell). In certain embodiments, the retroviral vector comprises an expression cassette that comprises one of the above-described insulators, e.g., insulator A1. The insulator can be positioned at the 3′ or the 5′ end of the expression cassette. In certain embodiments, the insulator is positioned at the 3′ end of the expression cassette. During reverse transcription and vector integration, the insulator positioned at the 3′ end is copied into the 5′ end of the expression cassette. The resulting topology places copies of the insulator between the genomic regions located at the 5′ LTR and the 3′ LTR of the integrated virus and enhancer activity from the 5′ LTR and internal package promoter, but does not contain the enhancer in the 3′ LTR. This topology can decrease genotoxicity, thereby resulting in decreased tumor formation and increased survival of the animals.

In certain embodiments, the vector is an Adeno-associated virus (AAV) vector. Non-limiting examples of AAV vectors include AAV1, AAV2, AAV3, AAV4, AAV5, AAV6, AAV7, AAV8, AAV9, AAV10, and AAV11. In certain embodiments, the vector is an AAV6 vector.

In certain embodiments, the recombinant vector is a retroviral vector. In certain embodiments, the retroviral vector is a lentiviral vector. In certain embodiments, the recombinant vector is a self-inactivating (SIN) lentiviral vector in which the enhancer and promoter region of the 3′ long terminal repeat (“LTR”) is modified (e.g., a 133 bp deletion in the U3 region). SIN vectors can infect and integrate into the genome of non-dividing cells in vivo with an efficacy similar to that of wild-type vectors. SIN lentivirus vectors are disclosed in Miyoshi et al., J. Virol. (October 1998); 72:8150-8157, which is incorporated by reference in its entirety. SIN vectors can reduce the risk of creating undesired replication-competent virus because there is no complete U3 region in the 3′ LTR, thereby eliminating the ability of the virus to be passed (see Miyoshi 1998).

In certain embodiments, the retroviral vector is a gamma-retroviral vector. In certain embodiments, the retroviral vector is a SIN gamma-retroviral vector.

In certain embodiments, the recombinant vector further comprises one or more of a rev-responsive element (“RRE”), a central polypurine tract (“cPPT”), a 5′ LTR (in which the U3 region of the 5′ LTR comprises a heterologous promoter (e.g., replaced with a cytomegalovirus (CMV) HIV heterologous promoter) resulting in Tat-independent transcription with no decreases in virus titer), and a 3′ SIN LTR (e.g., a 3′ SIN LTR comprising a 389 bp deletion in the U3 region).

In certain embodiments, the recombinant vector further comprises a Woodchuck hepatitis post-regulatory element (WPRE) in the 3′ long terminal repeat (LTR) of the vector (e.g., 3′ to the R region in the 3′ LTR of the vector). In certain embodiments, the recombinant vector further comprises a bovine growth hormone polyadenylation signal in addition to the WPRE in the 3′ long terminal repeat (LTR) of the vector (e.g., 3′ to the R region in the 3′ LTR of the vector). The WPRE can increase the titer of the recombinant vector (e.g., can increase the vector titer by at least about 5 folds). Addition of a bovine growth hormone (“BGH”) polyadenylation (“polyA”) signal (“BGH/polyA”) to the WPRE can further increase the titer of the recombinant vector. In certain embodiments, the WPRE and BGH/poly A are eliminated after the vector is transfused to target cells, and thus, are not present in the proviruses. In certain embodiments, the WPRE and the BGH/polyA are not comprised within the expression cassette, and thus, not transferred to the cells transduced with the recombinant vector.

As shown in FIGS. 1-4, 13, and 15, the recombinant vector can further comprises a rev-responsive element (“RRE”), a central polypurine tract (“cPPT”), a 5′ LTR (in which the U3 region of the 5′ LTR comprises a heterologous promoter (e.g., replaced with a cytomegalovirus (CMV) HIV heterologous promoter) resulting in Tat-independent transcription with no decreases in virus titer), a 3′ SIN LTR comprising a 389 bp deletion in the U3 region, a WPRE, and a BGH/poly A in the 3′ long terminal repeat (LTR) of the vector (e.g., 3′ to the R region in the 3′ LTR). The heterologous promoter (e.g., a CMV/HIVheterologous promoter) can increase the RNA transcription and the production of the vector. The heterologous promoter (e.g., a CMV/HIVheterologous promoter) is eliminated after reverse transcription, and thus, is not present in the proviruses.

In certain non-limiting embodiments, a presently disclosed expression cassette can be cloned into a retroviral vector and expression can be driven from its endogenous promoter, from the retroviral long terminal repeat, or from an alternative internal promoter. Combinations of retroviral vector and an appropriate packaging line are also suitable, where the capsid proteins will be functional for infecting human cells. Various amphotropic virus-producing cell lines are known, including, but not limited to, PA12 (Miller, et al. (1985) Mol. Cell. Biol. 5:431-437); PA317 (Miller, et al. (1986) Mol. (Cell. Biol. 6:2895-2902); and CRIP (Danos, et al. (1988) Proc. Natl. Acad. Sci. USA 85:6460-6464). Non-amphotropic particles are suitable too, e.g., particles pseudotyped with VSVG, RD114 or GALV envelope and any other known in the art.

Suitable methods of transduction also include direct co-culture of the cells with producer cells, e.g., by the method of Bregni, et al. (1992) Blood 80:141δ-1422, or culturing with viral supernatant alone or concentrated vector stocks with or without appropriate growth factors and polycations, e.g., by the method of Xu, et al. (1994) Exp. Hemat. 22:223-230; and Hughes, et al. (1992) J. Clin. Invest. 89:1817.

Transducing viral vectors can be used to express a globin gene (e.g., a human β-globin gene) in a host cell (e.g., hematopoietic stem cells, an embryonic stem cell, or an induced pluripotent stem cell). In certain embodiments, the chosen vector exhibits high efficiency of infection and stable integration and expression (see, e.g., Cayouette et al., Human Gene Therapy (1997); 8:423-430; Kido et al., Current Eye Research (1996); 15:833-844; Bloomer et al., Journal of Virology (1997); 71:6641-6649; Naldini et al., Science (1996); 272:263 267; and Miyoshi et al., Proc. Natl. Acad. Sci. U.S.A. 94:10319, 1997). Other viral vectors that can be used include, for example, adenoviral, lentiviral, and adeno-associated viral vectors, vaccinia virus, a bovine papilloma virus, or a herpes virus, such as Epstein-Barr Virus (also see, for example, the vectors of Miller, Human Gene Therapy (1990); 15-14; Friedman, Science (1989); 244:1275-1281; Eglitis et al., BioTechniques 6:608-614, 1988; Tolstoshev et al., Current Opinion in Biotechnology (1990); 1:55-61; Sharp, The Lancet (1991); 337: 1277-1278; Cornetta et al., Nucleic Acid Research and Molecular Biology (1987)36:311-322; Anderson, Science (1984); 226:401-409; Moen, Blood Cells (1991); 17:407-416; Miller et al., Biotechnology (1989); 7:980-990; Le Gal La Salle et al., Science (1993); 259:98δ-990; and Johnson, Chest (1995); 107:77S-83S). Retroviral vectors are particularly well developed and have been used in clinical settings (Rosenberg et al., N. Engl. J. Med (1990); 323:370; Anderson et al., U.S. Pat. No. 5,399,346).

The requirement for efficient delivery and integration make retroviral vectors suitable for transducing a presently disclosed expression cassette. Retroviral vectors can be derived from three genera of the retroviridae: the γ-retroviruses (also known as C-type murine retroviruses or oncoretroviruses), the lentiviruses, and the spumaviruses (also known as foamy viruses). Several reviews detailing molecular approaches for the generation of replication-defective retroviral particles are available (Cornetta et al. (2005); Cockrell & Kafri (2007)). The vector itself, which encodes the therapeutic transgene or cDNA, retains the minimal viral sequences needed to enable packaging in viral particles in a packaging cell line, reverse transcription, and integration. The packaging cell expresses the necessary structural proteins and enzymes that are required to assemble an infectious recombinant particle that contains the vector sequence and the machinery needed for its reverse transcription and integration in the transduced cell.

While the manufacturing aspects of all retroviral vector types follow the same general principles, γ-retroviral, lentiviral and spumaviral vectors differ in some of their intrinsic biological properties. Gamma-retroviruses, including the prototypic murine leukaemia viruses (MLV), effectively infect many cell types but are unable to integrate in cells that do not proceed to S phase soon after infection. In contrast, lentiviruses and their vector derivatives can transduce nondividing cells (Follenzi & Naldini, 2002; Salmon & Trono, 2002) owing to their ability to translocate to the nucleus and integrate in the absence of cell division (Lewis & Emerman, 1994; Goff, 2001). Another fundamental attribute of lentiviral vectors is their relative genomic stability, as established for globin lentiviral vectors (May et al., 2000), which contrasts with the genomic instability of MLV-based globin vectors (Leboulch et al., 1994; Sadelain et al., 1995). Lentiviral and foamy vectors further provide a greater packaging capacity (Kumar et al., 2001; Rethwilm, 2007). All three vector types have been used successfully for the transduction of cytokineactivated HSCs (Miyoshi et al., 1999; Josephson et al., 2002; Leurs et al., 2003).

These three vector systems differ in their integration patterns. The integration pattern of retroviruses is semi-random and biased towards genes and their vicinity in approximately two-thirds of all integration events (Schroder et al., 2002; Wu et al., 2003; Mitchell et al., 2004; De Palma et al., 2005; Trobridge et al., 2006). There are however subtle and possibly significant differences in their exact distribution. Gamma-retroviruses have a propensity to integrate upstream of transcribed genes, whereas lentiviruses and lentiviral vectors target the entire transcribed gene sequence. Foamy vectors appear to be less prone to intragenic integration (Trobridge et al., 2006). In certain embodiments, the vector comprising the expression cassette is a lentivirus vector. The vectors can be derived from human immunodeficiency-1 (HIV-1), human immunodeficiency-2 (HIV-2), simian immunodeficiency virus (SIV), feline immunodeficiency virus (FIV), bovine immunodeficiency virus (BIV), Jembrana Disease Virus (JDV), equine infectious anemia virus (EIAV), caprine arthritis encephalitis virus (CAEV) and the like. In one non-limiting embodiment, the lentiviral vector is an HIV vector. HIV-based constructs are the most efficient at transduction of human cells.

The semi-random pattern of vector integration exposes patients to the risk of insertional oncogenesis when the vector trans-activates a neighboring oncogene. This may result in clonal expansion (Ott et al, 2006; Cavazzana-Calvo et al, 2010), myelodysplasia (Stein et al, 2010) or leukaemia (Hacein-Bey-Abina et al, 2003, 2008; Howe et al, 2008). Targeted gene delivery strategies, utilizing a non-naturally occurring or engineered nuclease (including, but not limited to, Zinc-finger nuclease (ZNFs), meganuclease, transcription activator-like effector nuclease (TALEN)), or a CRISPR-Cas system, can reduce or even eliminate the concern of insertional oncogenesis that is inherent to the use of retroviral vectors.

Eukaryotic cells utilize two distinct DNA repair mechanisms in response to DNA double strand breaks (DSBs): Homologous recombination (HR) and non-homologous end-joining (NHEJ). The activation of the HR repair machinery depends on the cell cycle status, and it is restricted to the S and G2 phases; in contrast, the NHEJ pathway is active throughout the cell cycle. Mechanistically, HR is an error-free DNA repair mechanism, because it requires a homologous template to repair the damaged DNA strand. On the other hand, NHEJ is a template-independent repair mechanism that is imprecise, due to DNA end processing during repair that leads to insertions or deletions at the DNA break site (Moynahan & Jasin, 2010). Because of its homology-based mechanism, HR has been used as a tool to site-specifically engineer the genome of different species. From a therapeutic perspective, HR has been successfully used to repair mutated genes, thus offering a promising approach to cell-mediated treatment of monogenic diseases (Porteus et al, 2006).

Gene targeting by HR requires the use of two homology arms that flank the transgene/target site of interest. Generally, standard plasmid DNAs have been used to deliver 5-10 kb homology arms along with transgenes for positive and negative selection. This method is commonly used to knock-out/knockin genes in mouse embryonic stem (mES) cells (Capecchi, 2005; FIG. 2B). In human cells, the use of this approach has allowed gene targeting with efficiencies in the order of 10-6, which are lower than in mES cells and are not therapeutically practical. HR efficiency can be increased by the introduction of DNA-doubled stranded breaks (DSBs) at the target site using specific rare-cutting endonucleases, resulting in over 1,000-fold increase in correct gene targeting (Jasin, 1996). The discovery of this phenomenon prompted the development of methods to create site-specific DSBs in the genome of different species. Various chimeric enzymes have been designed for this purpose over the last decade, namely zinc-finger nucleases (ZFNs), meganucleases, and transcription activator-like effector nucleases (TALENs).

ZFNs are modular chimeric proteins that contain a ZF-based DNA binding domain (DBD) and a FokI nuclease domain (Porteus & Carroll, 2005). DBD is usually composed of three ZF domains, each with 3-base pair specificity; the FokI nuclease domain provides a DNA nicking activity, which is targeted by two flanking ZFNs. Owing to the modular nature of the DBD, any site in a genome could be targeted in principle. However, as a single ZFN can bind and nick DNA, there is potential for a high number of off-target effects, resulting in the activation of the NHEJ pathway that may either introduce insertions/deletions or integrate the targeting vector in a non-specific manner. Obligate FokI domains that can nick their respective DNA strand only when they form a heterodimer were recently reported (Doyon et al, 2011). The use of such obligate ZFNs can reduce the genotoxic effects of this approach.

Meganucleases (MNs)/homing endonucleases (HEs) are dsDNA nucleases that recognize and cleave large DNA sites (14-40 bp) with low cleavage frequencies in eukaryotic genomes (Paques & Duchateau, 2007). Although this limits the potential target sites, MN-DNA structures have been used as a guide to specifically modify DNA-interacting residues in order to change the MN specificity (Marcaida et al, 2010). I-Crel has been successfully engineered to generate chimeric meganucleases that target the human XPC and RAGI genes, and they have been shown to stimulate HR activity in mammalian cells with no evident genotoxicity (Redondo et al, 2008; Grizot et al, 2009). The genotoxicity of this approach will need to be compared to that of ZFNs and TALE nucleases. TALENs are similar ZFN except that the DBD is derived from transcription activator-like effectors (TALEs), which are virulent factors used by phytopathogenic bacteria (Herbers, 1992). The TALE DBD is modular, and it is composed of 34-residue repeats, and its DNA specificity is determined by the number and order of repeats (Herbers, 1992). Each repeat binds a single nucleotide in the target sequence through only two residues (Boch, 2011). The advantage over ZFN technology is the rapid construction of DBDs.

A number of studies have used these chimeric enzymes to stimulate HR for either gene addition or gene repair at their target site (Paques & Duchateau, 2007; Urnov et al, 2010). Porteus designed a ZFN to a half site sequence from the human HBB that surrounds the sickle cell mutation nucleotide (Porteus, 2006). This ZFN targets the sequence and stimulates HR at a chimeric DNA target when combined with a ZFN targeting the Zif268 binding site. There have been recent advances in targeting genes in cord blood CD34⁺ cells. Use of non-integrating lentiviruses to deliver ZFNs and the donor DNA in these cells to target the CCR5 gene was reported in Lombardo et al, 2007. Lombardo et al, 2007 showed gene addition at this locus with correct targeting in 80% of the positively selected cells.

The presently disclosed subject matter provides a non-naturally occurring or engineered nuclease comprising a presently disclosed expression cassette, as described above. Suitable nucleases include, but are not limited to, ZFNs, meganucleases, and TALENs. A presently disclosed nuclease comprises a DNA binding domain and a nuclease cleavage domain. The DNA binding domain of the nuclease can be engineered to bind to a sequence of choice, e.g., a predetermined site. An engineered DNA binding domain can have a distinct binding specificity, compared to a naturally occurring nuclease. Engineering methods include, but are not limited to, rational design and various types of selection. Any suitable cleavage domain can be operatively linked to a DNA-binding domain to form a nuclease. For example, Zinc-finger protein (ZFP) DNA-binding domains can be fused to nuclease cleavage domains to create ZFNs-a functional entity that is able to recognize its intended nucleic acid target through its engineered ZFP DNA binding domain and cause the DNA to be cut near the ZFP binding site via the nuclease activity. See, e.g., Kim et al. Proc Nat'l Acad Sci USA (1996); 93(3):1156-1160. Likewise, TALE DNA-binding domains can be fused to nuclease cleavage domains to create TALENs. See, e.g., U.S. Publication No. 20110301073.

The cleavage domain can be heterologous to the DNA-binding domain, e.g., a meganuclease DNA-binding domain and cleavage domain from a different nuclease. Heterologous cleavage domains can be obtained from any endonuclease or exonuclease. Exemplary endonucleases from which a cleavage domain can be derived include, but are not limited to, restriction endonucleases and homing endonucleases. See, for example, 2002-2003 Catalog, New England Biolabs, Beverly, Mass.; and Belfort et al. (1997) Nucleic Acids Res. 25:3379-3388. Additional enzymes which cleave DNA are known (e.g., S1 Nuclease; mung bean nuclease; pancreatic DNase I; micrococcal nuclease; yeast HO endonuclease; see also Linn et al. (eds.) Nucleases, Cold Spring Harbor Laboratory Press, 1993). One or more of these enzymes (or functional regions thereof) can be used as a source of cleavage domains and cleavage half-domains.

Similarly, a cleavage half-domain can be derived from the above-described nuclease that requires dimerization for cleavage activity. In general, two fusion proteins are required for cleavage if the fusion proteins comprise cleavage half-domains. Alternatively, a single protein comprising two cleavage half-domains can be used. The two cleavage half-domains can be derived from the same endonuclease (or functional portions thereof), or each cleavage half-domain can be derived from a different endonuclease (or functional portions thereof).

In certain embodiments, the nuclease comprises an expression cassette that comprises two of the insulators disclosed herein, e.g., two of the insulator having the nucleotide sequence set forth in SEQ ID NO: 1, SEQ ID NO: 24, SEQ ID NO: 25. SEQ ID NO: 27. SEQ ID NO: 28. SEQ ID NO: 29, SEQ ID NO: 30, or SEQ ID NO: 31. One of the two insulators is positioned at the 3′ end of the expression cassette, and the other insulator is positioned at the 5′ end of the expression cassette.

The presently disclosed subject matter also provides a non-naturally occurring or engineer CRISPR-Cas system comprising the above-described expression cassette. The CRISPR (Clustered Regularly Interspaced Short Palindromic Repeats)-Cas (CRISPR Associated) system is an engineered nuclease system based on a bacterial system that can be used for genome engineering. It is based on part of the adaptive immune response of many bacteria and archea. When a virus or plasmid invades a bacterium, segments of the invader's DNA are converted into CRISPR RNAs (crRNA) by the “immune” response. The crRNA then associates, through a region of partial complementarity, with another type of RNA called tracrRNA to guide a CRISPR-Cas nuclease to a region homologous to the crRNA in the target DNA called a “proto spacer”. The CRISPR-Cas nuclease cleaves the DNA to generate blunt ends at the DSB at sites specified by a 20-nucleotide guide sequence contained within the crRNA transcript. The CRISPR-Cas nuclease requires both the crRNA and the tracrRNA for site specific DNA recognition and cleavage. This system has been engineered such that the crRNA and tracrRNA can be combined into one molecule (the “single guide RNA”); and the crRNA equivalent portion of the single guide RNA can be engineered to guide the CRISPR-Cas nuclease to target any desired sequence (see Jinek et al., Science (2012); 337:816-821). Thus, the CRISPR-Cas system can be engineered to create a DSB at a desired target in a genome. In certain embodiments, the CRISPR-Cas system comprises a CRISPR-Cas nuclease and a single-guide RNA. Suitable examples of CRISPR-Cas nucleases include, but are not limited to, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These CRISPR-Cas nucleases are known; for example, the amino acid sequence of S. pyogenes Cas9 protein may be found in the SwissProt database under accession number Q99ZW2. In some embodiments, the CRISPR-Cas nuclease has DNA cleavage activity, e.g., Cas9. In certain embodiments, the CRISPR-Cas nuclease is Cas9. The CRISPR-Cas nuclease can direct cleavage of one or both strands at the location of a target sequence (e.g., a genomic safe harbor site). Additionally, the CRISPR-Cas nuclease can direct cleavage of one or both strands within about 1, 2, 3, 4, 5, 6, 7, 8, 9, 10, 15, 20, 25, 50, 100, 200, 500, or more base pairs from the first or last nucleotide of a target sequence.

The presently disclosed nucleases and CRISPR-Cas system allow for targeted delivery of the expression cassette. In certain embodiments, a presently disclosed CRISPR-Cas system or the DNA binding domain of a presently disclosed nuclease binds to a genomic safe harbor site. A nuclease or the CRISPR-Cas system generates a double strand break at the genomic safe harbor site. Genomic safe harbor sites are intragenic or extragenic regions of the human genome that are able to accommodate the predictable expression of newly integrated DNA without adverse effects on the host cell or organism. A useful safe harbor must permit sufficient transgene expression to yield desired levels of the vector-encoded protein or non-coding RNA. A genomic safe harbor site also must not predispose cells to malignant transformation nor alter cellular functions. Methods for identifying genomic safe harbor sites are described in Sadelain et al., “Safe Harbours for the integration of new DNA in the human genome,” Nature Reviews (2012); 12:51-58; Papapetrou et al., “Genomic safe harbors permit high β-globin transgene expression in thalassemia induced pluripotent stem cells” Nat Biotechnol. (2011) January; 29(1):73-8, which are incorporated by reference in their entireties. A presently disclosed genomic safe harbor site meets one or more (one, two, three, four, or five) of the following five criteria: (1) distance of at least 50 kb from the 5′ end of any gene (e.g., from the 5′ end of the gene), (ii) distance of at least 300 kb from any cancer-related gene, (iii) within an open/accessible chromatin structure (measured by DNA cleavage with natural or engineered nucleases), (iv) location outside a gene transcription unit and (v) location outside ultraconserved regions (UCRs), microRNA or long non-coding RNA of the human genome. As the most common insertional oncogenesis event is transactivation of neighboring tumor-promoting genes, the first two criteria exclude the portion of the human genome located near promoters of genes, in particular, cancer-related genes, which are genes functionally implicated in human cancers or the human homologs of genes implicated in cancer in model organisms. Proximity to miRNA genes is one exclusion criterion because miRNAs are implicated in the regulation of many cellular processes, including cell proliferation and differentiation. As vector integration within a transcription unit can disrupt gene function through the loss of function of a tumor suppressor gene or the generation of an aberrantly spliced gene product, the fourth (iv) criterion excludes all sites located inside transcribed genes. UCRs, which are regions that are highly conserved over multiple vertebrates and known to be enriched for enhancers and exons, and long non-coding RNAs, are also excluded. In certain embodiments, the genomic safe harbor site is an extragenic genomic safe harbor site. In certain embodiments, the genomic safe harbor site is located on chromosome 1.

The presently disclosed subject matter also provides polynucleotides encoding the above-described nucleases, vectors comprising the polynucleotides encoding the above-described nucleases, polynucleotides encoding the above-described CRISPR-Cas system, and vectors comprising the polynucleotides encoding the above-described CRISPR-Cas system.

The nucleases and polynucleotides encoding these nucleases, and the CRISPR-Cas system and polynucleotides encoding the CRISPR-Cas system can be delivered in vivo or ex vivo by any suitable means. For example, nucleases and CRISPR-Cas system as described herein can be delivered to a cell (e.g., a hematopoietic stem cell, an embryonic stem cell, an induced pluripotent stem cell, or an hemogenic endothelium cell) by a vector comprising polynucleotides encoding the nuclease or the CRISPR-Cas system. Any vectors can be used including, but not limited to, plasmid vectors, retroviral vectors (e.g., γ-retroviral vectors, lentiviral vectors and foamy viral vectors), adenovirus vectors, poxvirus vectors; herpes virus vectors and adena-associated virus vectors, etc. In certain embodiments, the vector comprising a polynucleotide encoding an above-described nuclease or an above-described CRISPR-Cas system is a lentiviral vector. In one particular embodiment, the lentiviral vector is a non-integrating lentiviral vector. Examples of non-integrating lentiviral vector are described in Ory et al. (1996) Proc. Natl. Acad. Sci. USA 93:11382-11388; Dull et al., (1998) J. Viral. 72:8463-8471; Zuffery et al. (1998) J. Viral. 72:9873-9880; Follenzi et al., (2000) Nature Genetics 25:217-222; U.S. Patent Publication No 2009/054985.

Additionally, non-viral approaches (e.g., single-stranded DNA) can also be employed for the expression of a globin gene in cells. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases. Transient expression may be obtained by RNA electroporation.

### IV. Cells

Genetic modification of cells (e.g., hematopoietic stem cells, embryonic stem cells, induced pluripotent stem cells, and hemogenic endothelium cells) can be accomplished by transducing a substantially homogeneous cell composition with a recombinant DNA or RNA construct (e.g., a vector or a delivery system comprising the above-described expression cassette). The presently disclosed subject matter provides cells transduced with the above-described expression cassettes, cells transduced with the above-described vectors, and cells transduced with the above-described nucleases or with vectors comprising polynucleotides encoding the nucleases, and cell transduced with the above-described CARISPR-Cas system or with vectors comprising polynucleotides encoding the CARISPR-Cas system, which are collectively referred to as “transduced cells”. As described above, the vectors, nucleases and CRISPR-Cas system are employed for transduction of the expression cassette to the cells to express a globin gene (e.g., a human β-globin gene). In certain embodiments, the transduced cells are administered to a subject to treat and/or prevent a hematopoietic disease, disorder, or condition. The presently disclosed insulators can enhance the efficiency of the transduction of the expression cassette to cells.

Suitable transduced cells include, but are not limited to, stem cells, progenitor cells, and differentiated cells. As used herein, the term “progenitor” or “progenitor cells” refers to cells that have the capacity to self-renew and to differentiate into more mature cells. Progenitor cells have a reduced potency compared to pluripotent and multipotent stem cells. Many progenitor cells differentiate along a single lineage, but may also have quite extensive proliferative capacity.

In certain embodiments, the transduced cells are stem cells. Stem cells have the ability to differentiate into the appropriate cell types when administered to a particular biological niche, in vivo. A stem cell is an undifferentiated cell capable of (1) long term self-renewal, or the ability to generate at least one identical copy of the original cell, (2) differentiation at the single cell level into multiple, and in some instance only one, specialized cell type and (3) of in vivo functional regeneration of tissues. Stem cells are sub-classified according to their developmental potential as totipotent, pluripotent, multipotent and oligo/unipotent. As used herein, the term “pluripotent” means the ability of a cell to form all lineages of the body or soma (i.e., the embryo proper). For example, embryonic stem cells are a type of pluripotent stem cells that are able to form cells from each of the three germs layers, the ectoderm, the mesoderm, and the endoderm. As used herein, the term “multipotent” refers to the ability of an adult stem cell to form multiple cell types of one lineage. For example, hematopoietic stem cells are capable of forming all cells of the blood cell lineage, e.g., lymphoid and myeloid cells.

In certain embodiments, the transduced cells are embryonic stem cells, bone marrow stem cells, umbilical cord stem cells, placental stem cells, mesenchymal stem cells, neural stem cells, liver stem cells, pancreatic stem cells, cardiac stem cells, kidney stem cells, and/or hematopoietic stem cells. In certain embodiments, the transduced cells are hematopoietic stem cells (HSCs). HSCs give rise to committed hematopoietic progenitor cells (HPCs) that are capable of generating the entire repertoire of mature blood cells over the lifetime of an organism. The term “hematopoietic stem cell” or “HSC” refers to multipotent stem cells that give rise to all blood cell types of an organism, including myeloid (e.g., monocytes and macrophages, neutrophils, basophils, eosinophils, erythrocytes, megakaryocytes/platelets, dendritic cells), and lymphoid lineages (e.g., T-cells, B-cells, NK-cells). When transplanted into lethally irradiated animals or humans, hematopoietic stem and progenitor cells can repopulate the erythroid, neutrophil-macrophage, megakaryocyte and lymphoid hematopoietic cell pool.

HSCs can be isolated or collected from bone marrow, umbilical cord blood, or peripheral blood. HSCs can be identified according to certain phenotypic or genotypic markers. For example, HSCs can be identified by their small size, lack of lineage (lin) markers, low staining (side population) with vital dyes such as rhodamine 123 (rhodamineDULL, also called rholo) or Hoechst 33342, and presence of various antigenic markers on their surface, many of which belong to the cluster of differentiation series (e.g., CD34, CD38, CD90, CD133, CD105, CD45, Terl 19, and c-kit, the receptor for stem cell factor). In certain embodiments, the transduced cell is a CD34 HSC.

In certain embodiments, the transduced cell is an embryonic stem cell. In certain embodiments, the transduced cell is an induced pluripotent stem cell. In certain embodiments, the transduced cell is a hemogenic endothelium cell.

While HSCs are the natural vehicle for restoring long-term hematopoiesis, their use has some important limitations. The first is their relative scarcity, which can eventually preclude autologous HSC therapy when the harvested cellular product is too small. The second is the difficulty to perform biosafety testing such as integration site analysis and consequently to select cells with chosen integration sites, because adult HSCs cannot be replicated in vitro. The third limitation is that homologous recombination using current technologies is practically impossible thus compromising the advent of gene correction. All of these limitations are ultimately due to the fact that adult HSCs cannot be expanded in vitro without losing their stem cell potency. These limitations explain the importance of viral vectors such as gamma-retroviral and lentiviral vectors, which are remarkably quick and efficient in achieving stable gene transfer.

Use of ESs and induced pluripotent stem (iPS) cells for globin gene therapy is disclosed in Moi et al., Haematol Mar. 1, 2008; 93(3):325-330. Embryonic stem (ES) cells are amenable to gene targeting and correction, which requires unlimited in vitro cell division without losing multipotency. Chang et al., Proc Natl Acad Sci USA 2006; 103:1036-40 provided proof of principle of the feasibility of the homologous recombination approach in mice with sickle cell anemia. Takahashi et al. Cell 2006; 126:663-76 reported the successful reprogramming of fibroblasts to an embryonic stem-like state. Cells obtained by this reverse-differentiation process, called induced pluripotent stem (iPS) cells, were produced by exposing embryonic or young adult bulk fibroblast cultures to gamma-retroviral vectors encoding 4 transcription factors, which are physiologically active in the embryonic stem cells, but generally turned off when differentiation progresses. The cultured cells formed colonies similar to ES cell colonies. These findings have been confirmed and extended by others to both mouse and human fibroblasts (Meissner et al., Nat Biotechnol 2007; 25:1177-81; Nakagawa et al., Nat Biotechnol 2007; 26:101-6; Okita et al., Nature 2007; 448:313-7; Park et al., Nature 2007; 451:141-6; Takahashi et al., Nat Protoc 2007; 2:3081-9; Takahashi K et al., Cell 2007; 131:861-72; Wernig et al., Nature 2007; 448:318-24; Yu J et al., Science 2007; 318: 1917-20). Rudolf Jaenisch and co-workers achieved a successful gene therapy in a mouse model of sickle cell disease, using homologous recombination in ES-like iPS cells (Hanna et al., Science 2007; 318:1920-3). The process has so far been mostly applied to fibroblast harvested from a skin biopsy, which are then induced to become iPS by transduction with retroviral vectors that encode four stem cell transcription factors. iPS are amenable to the correction of the SC mutation by standard homologous recombination techniques and can then be differentiated in vitro into unlimited amounts of hematopoietic stem cells. The whole process ends with the autologous transplantation of the corrected HSC into the original mouse donor, which will now be cured of its SC disease. This technique is not only useful for homologous recombination, but can also enhance lentiviral-mediated globin gene transfer for the treatment of β-thalassemia by providing a means to perform detailed integration site analysis and adequate in vitro cell expansion before infusing cells into the recipient.

The cell of the presently disclosed subject matter can be autologous (“self”) or non-autologous (“non-self,” e.g., allogeneic, syngeneic or xenogeneic). As used herein, “autologous” refers to cells from the same subject. As used herein, “allogeneic” refers to cells of the same species that differ genetically to the cell in comparison. As used herein, “syngeneic” refers to cells of a different subject that are genetically identical to the cell in comparison. As used herein, “xenogeneic” refers to cells of a different species to the cell in comparison. In certain embodiments, the cell is autologous, e.g., a cell transduced with the presently disclosed expression cassette is administered to a subject from whom the cell is collected, e.g., the cell is collected from bone marrow, umbilical cord blood, peripheral blood, and/or adipose tissue of the subject. In certain embodiments, the cell is obtained or collected from bone marrow of a subject.

In certain embodiments, prior to transduction with the expression cassette, the cell is pre-stimulated, e.g., in the presence of one or more cytokines (e.g., IL-3, IL-la, IL-6, Kit ligand (also known as “Stem Cell Factor (SCF)”), and Flt-3 ligand), and/or one or more glycoproteins (e.g., thrombopoietin and fibronectin). In certain embodiments, the cell is pre-stimulated in the presence of Flt-3 ligand, SCF, thrombopoietin, interleukin-3, and fibronectin. The cell can be pre-stimulated for about 24 hours or longer, e.g., about 48 hours, or about 36 hours. Subsequently, the cell is transduced with a presently disclosed expression cassette, or a vector or another delivery system comprising such expression cassette. Transduction can be performed on a fresh cell, or on a frozen cell. Genomic DNA of the cell is isolated to determine the vector copy number and analyze the integration site or integrated vector structure, e.g., by South blot analysis and/or by Quantitative PCR. For quantification of globin mRNA (e.g., human β-globin transgene analysis), total RNA is extracted from the cell. Quantitative primer extension assay can be used for quantification of globin mRNA.

### V. Compositions and Formulations

The presently disclosed subject matter provides pharmaceutical compositions comprising a presently disclosed transduced cell as described above and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible, including pharmaceutically acceptable cell culture media. The pharmaceutically acceptable carrier can be suitable for parenteral (e.g., intravenous, intramuscular, subcutaneous, or intraperitoneal), spinal or epidermal administration (e.g., by injection, infusion or implantation). Depending on the route of administration, the active compound, e.g., the transduced cell, may be coated in a material to protect the compound from the action of acids and other natural conditions that may inactivate the compound.

Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the transduced cells, use thereof in the pharmaceutical compositions of the invention is contemplated.

The pharmaceutical compositions of the presently disclosed subject matter can further comprise one or more polypeptides, polynucleotides, vectors comprising the same, transduced cells, etc., as described herein, formulated in pharmaceutically-acceptable or physiologically-acceptable solutions for administration to a cell or an animal, either alone, or in combination with one or more other modalities of therapy. If desired, the pharmaceutical compositions of the presently disclosed subject matter can be administered in combination with other agents, including, but not limited to, cytokines, growth factors, hormones, small molecules or various pharmaceutically-active agents. Any additional agents that do not adversely affect the ability of the composition to deliver the intended gene therapy can be included in the compositions.

In the pharmaceutical compositions of the presently disclosed subject matter, formulation of pharmaceutically-acceptable excipients and carrier solutions is well known to those of ordinary skill in the art, as is the development of suitable dosing and treatment regimens for using the particular compositions described herein in a variety of treatment regimens, including, e.g., oral, parenteral, intravenous, intranasal, and intramuscular administration and formulation.

The pharmaceutical compositions of the presently disclosed subject matter can be delivered parenterally (e.g., intravenously, intramuscularly, or intraperitoneally) as described, for example, in U.S. Pat. Nos. 5,543,158; 5,641,515 and 5,399,363. Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The pharmaceutically acceptable carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

The pharmaceutical compositions of the presently disclosed subject matter can be conveniently provided as sterile liquid preparations, e.g., isotonic aqueous solutions, suspensions, emulsions, dispersions, or viscous compositions, which can be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the compositions of the presently disclosed subject matter in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, alum inurn monostearate and gelatin.

The compositions can be isotonic, i.e., they can have the same osmotic pressure as blood and lacrimal fluid. The desired isotonicity of the compositions of the presently disclosed subject matter can be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

For parenteral administration in an aqueous solution, for example, the solution should be suitably buffered if necessary and the liquid diluent first rendered isotonic with sufficient saline or glucose. Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

In certain embodiments, the compositions can be delivered by intranasal sprays, inhalation, and/or other aerosol delivery vehicles. Methods for delivering genes, polynucleotides, and peptide compositions directly to the lungs via nasal aerosol sprays are described, e.g., in U.S. Pat. Nos. 5,756,353 and 5,804,212. Methods of delivering drugs using lysophosphatidyl-glycerol compounds are described, e.g., in U.S. Pat. No. 5,725,871. Transmucosal drug delivery in the form of a polytetrafluoroetheylene support matrix is described, e.g., in U.S. Pat. No. 5,780,045. The compositions of the presently disclosed subject matter can be formulated for delivery either encapsulated in a lipid particle, a liposome, a vesicle, a nanosphere, a nanoparticle or the like. The formulation and use of such delivery vehicles can be carried out using known and conventional techniques. The formulations and compositions of the presently disclosed subject matter can comprise one or more repressors and/or activators comprising a combination of any number of polypeptides, polynucleotides, and small molecules, as described herein, formulated in pharmaceutically-acceptable or physiologically-acceptable solutions (e.g., culture medium) for administration to a cell or an animal, either alone, or in combination with one or more other modalities of therapy.

In certain aspects, the presently disclosed subject matter provides formulations or compositions suitable for the delivery of viral vector systems (i.e., viral-mediated transduction) including, but not limited to, retroviral (e.g., lentiviral) vectors. Exemplary formulations for ex vivo delivery can also include the use of various transfection agents known in the art, such as calcium phosphate, electoporation, heat shock and various liposome formulations (i.e., lipid-mediated transfection). Liposomes are lipid bilayers entrapping a fraction of aqueous fluid. DNA spontaneously associates to the external surface of cationic liposomes (by virtue of its charge) and these liposomes will interact with the cell membrane.

The skilled artisan can readily determine the amount of cells and optional additives, vehicles, and/or carrier in compositions and to be administered in methods of the presently disclosed subject matter. Typically, any additives (in addition to the transduced cell(s) and/or agent(s)) are present in an amount of from about 0.001% to about 50% by weight) solution in phosphate buffered saline, and the active ingredient is present in the order of micrograms to milligrams, such as from about 0.0001 wt % to about 5 wt %, from about 0.0001 wt % to about 1 wt %, from about 0.0001 wt % to about 0.05 wt %, from about 0.001 wt % to about 20 wt %, from about 0.01 wt % to about 10 wt %, or from about 0.05 wt % to about 5 wt %. For any composition to be administered to an animal or human, and for any particular method of administration, toxicity should be determined, such as by determining the lethal dose (LD) and LD50 in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

### VI. Uses and Methods

Vectors and other delivery systems (nucleases and CRISPR-Cas systems) comprising the presently disclosed expression cassette provide improved methods of gene therapy. As used herein, the term “gene therapy” refers to the introduction of a polynucleotide into a cell's genome that restores, corrects, or modifies the gene and/or expression of the gene. In various non-limiting embodiments, a presently disclosed vector or other delivery system (e.g., a nuclease or a CRISPR-Cas system) comprises an expression cassette comprising a globin gene or a functional portion thereof that encodes a globin protein (e.g., human β globin protein), which provides curative, preventative, or ameliorative benefits to a subject diagnosed with or that is suspected of having a disease, disorder, or condition of the hematopoietic system. The vector or other delivery systems (e.g., a nuclease and the CRISPR-Cas system) can infect and transduce the cell in vivo, ex vivo, or in vitro. In ex vivo and in vitro embodiments, the transduced cells can then be administered to a subject in need of therapy. The presently disclosed subject matter contemplates that the vectors and other delivery systems (e.g., nucleases or CRISPR-Cas systems), viral particles, and transduced cells of the presently disclosed subject matter are be used to treat, prevent, and/or ameliorate a disease, disorder, or condition of the hematopoietic system in a subject, e.g., a hemoglobinopathy.

As used herein, the term “hemoglobinopathy” or “hemoglobinopathic condition” includes any disorder involving the presence of an abnormal hemoglobin molecule in the blood. Examples of hemoglobinopathies included, but are not limited to, hemoglobin C disease, hemoglobin sickle cell disease (SCD), sickle cell anemia, and thalassemias. Also included are hemoglobinopathies in which a combination of abnormal hemoglobins are present in the blood (e.g., sickle cell/Hb-C disease). As used herein, “thalassemia” refers to a hereditary disorder characterized by defective production of hemoglobin. Examples of thalassemias include α- and β-thalassemia. β-thalassemias are caused by a mutation in the beta globin chain, and can occur in a major or minor form. In the major form of β-thalassemia, children are normal at birth, but develop anemia during the first year of life. The mild form of β-thalassemia produces small red blood cells and the thalassemias are caused by deletion of a gene or genes from the globin chain. α-thalassemia typically results from deletions involving the HBA1 and HBA2 genes. Both of these genes encode α-globin, which is a component (subunit) of hemoglobin. There are two copies of the HBA1 gene and two copies of the HBA2 gene in each cellular genome. As a result, there are four alleles that produce α-globin. The different types of a thalassemia result from the loss of some or all of these alleles. Hb Bart syndrome, the most severe form of a thalassemia, results from the loss of all four α-globin alleles. HbH disease is caused by a loss of three of the four [alpha]-globin alleles. In these two conditions, a shortage of [alpha]-globin prevents cells from making normal hemoglobin. Instead, cells produce abnormal forms of hemoglobin called hemoglobin Bart (Hb Bart) or hemoglobin H (HbH). These abnormal hemoglobin molecules cannot effectively carry oxygen to the body's tissues. The substitution of Hb Bart or HbH for normal hemoglobin causes anemia and the other serious health problems associated with a thalassemia. As used herein, the term “sickle cell disease” refers to a group of autosomal recessive genetic blood disorders, which results from mutations in a globin gene and which is characterized by red blood cells that assume an abnormal, rigid, sickle shape. They are defined by the presence of BS-gene coding for a β-globin chain variant in which glutamic acid is substituted by valine at amino acid position 6 of the peptide, and second β-gene that has a mutation that allows for the crystallization of HbS leading to a clinical phenotype. As used herein, the term “sickle cell anemia” refers to a specific form of sickle cell disease in patients who are homozygous for the mutation that causes HbS. Other common forms of sickle cell disease include HbS/β-thalassemia, HbS/HbC and HbS/HbD.

In certain embodiments, gene therapy methods of the presently disclosed subject mater are used to treat, prevent, or ameliorate a hemoglobinopathy that is selected from the group consisting of: hemoglobin C disease, hemoglobin sickle cell disease (SCD), sickle cell anemia, hereditary anemia, thalassemia, β-thalassemia, thalassemia major, thalassemia intermedia, α-thalassemia, and hemoglobin H disease. In certain embodiments, the hemoglobinopathy is β-thalassemia. In certain embodiments, the hemoglobinopathy is sickle cell anemia

In various non-limiting embodiments, vectors or other delivery systems (e.g., nucleases or CRISPR-Cas systems) comprising a presently disclosed expression cassette are administered by direct injection to a cell, tissue, or organ of a subject in need of gene therapy, in vivo. In certain embodiments, cells are transduced in vitro or ex vivo with vectors or other delivery systems (e.g., nucleases or CRISPR-Cas systems) of the presently disclosed subject matter, and optionally expanded ex vivo. The transduced cells are then administered to a subject in need of gene therapy, e.g., within a pharmaceutical formulation disclosed herein.

The presently disclosed subject matter provides a method of providing a transduced cell to a subject. In various non-limiting embodiments, the method comprises administering (e.g., parenterally) one or more cells (a population of cells) transduced with a presently disclosed expression cassette or a vector or another delivery system (e.g., a nuclease or CRISPR-Cas system) comprising such expression cassette to the subject.

The presently disclosed subject matter provides a method of treating a hemoglobinopathy in a subject. In various non-limiting embodiments, the method comprises administering an effective amount of a presently disclosed transduced cell or a population of the presently disclosed transduced cells (e.g., HSCs, embryonic stem cells, or iPSCs) to the subject.

For treatment, the amount administered is an amount effective in producing the desired effect. An effective amount can be provided in one or a series of administrations. An effective amount can be provided in a bolus or by continuous perfusion. An “effective amount” (or “therapeutically effective amount”) is an amount sufficient to affect a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the immunoresponsive cells administered.

In certain embodiments, following administration of one or more of the presently disclosed transduced cells, peripheral blood of the subject is collected and hemoglobin levels is measured. A therapeutically relevant level of hemoglobin is produced following administration of one or more of the presently disclosed transduced cells. Therapeutically relevant level of hemoglobin is a level of hemoglobin that is sufficient (1) to improve or correct anemia, (2) to restore the ability of the subject to produce red blood cells containing normal hemoglobin, (3) to correct ineffective erythropoiesis in the subject, (4) to correct extra-medullary hematopoiesis (e.g., splenic and hepatic extra-medullary hematopoiesis), and/or (5) to reduce iron accumulation, e.g., in peripheral tissues and organs. Therapeutically relevant level of hemoglobin can be at least about 7 g/dL Hb, at least about 7.5 g/dL Hb, at least about 8 g/dL Hb, at least about 8.5 g/dL Hb, at least about 9 g/dL Hb, at least about 9.5 g/dL Hb, at least about 10 g/dL Hb, at least about 10.5 g/dL Hb, at least about 11 g/dL Hb, at least about 11.5 g/dL Hb, at least about 12 g/dL Hb, at least about 12.5 g/dL Hb, at least about 13 g/dL Hb, at least about 13.5 g/dL Hb, at least about 14 g/dL Hb, at least about 14.5 g/dL Hb, or at least about 15 g/dL Hb. Additionally or alternatively, therapeutically relevant level of hemoglobin can be from about 7 g/dL Hb to about 7.5 g/dL Hb, from about 7.5 g/dL Hb to about 8 g/dL Hb, from about 8 g/dL Hb to about 8.5 g/dL Hb, from about 8.5 g/dL Hb to about 9 g/dL Hb, from about 9 g/dL Hb to about 9.5 g/dL Hb, from about 9.5 g/dL Hb to about 10 g/dL Hb, from about 10 g/dL Hb to about 10.5 g/dL Hb, from about 10.5 g/dL Hb to about 11 g/dL Hb, from about 11 g/dL Hb to about 11.5 g/dL Hb, from about 11.5 g/dL Hb to about 12 g/dL Hb, from about 12 g/dL Hb to about 12.5 g/dL Hb, from about 12.5 g/dL Hb to about 13 g/dL Hb, from about 13 g/dL Hb to about 13.5 g/dL Hb, from about 13.5 g/dL Hb to about 14 g/dL Hb, from about 14 g/dL Hb to about 14.5 g/dL Hb, from about 14.5 g/dL Hb to about 15 g/dL Hb, from about 7 g/dL Hb to about 8 g/dL Hb, from about 8 g/dL Hb to about 9 g/dL Hb, from about 9 g/dL Hb to about 10 g/dL Hb, from about 10 g/dL Hb to about 11 g/dL Hb, from about 11 g/dL Hb to about 12 g/dL Hb, from about 12 g/dL Hb to about 13 g/dL Hb, from about 13 g/dL Hb to about 14 g/dL Hb, from about 14 g/dL Hb to about 15 g/dL Hb, from about 7 g/dL Hb to about 9 g/dL Hb, from about 9 g/dL Hb to about 11 g/dL Hb, from about 11 g/dL Hb to about 13 g/dL Hb, or from about 13 g/dL Hb to about 15 g/dL Hb. In certain embodiments, the therapeutically relevant level of hemoglobin is maintained in the subject for at least about 6 months, for at least about 12 months (or 1 year), for at least about 24 months (or 2 years). In certain embodiments, the therapeutically relevant level of hemoglobin is maintained in the subject for up to about 6 months, for up to about 12 months (or 1 year), for up to about 24 months (or 2 years). In certain embodiments, the therapeutically relevant level of hemoglobin is maintained in the subject for about 6 months, for about 12 months (or 1 year), for about 24 months (or 2 years). In certain embodiments, the therapeutically relevant level of hemoglobin is maintained in the subject for from about 6 months to about 12 months (e.g., from about 6 months to about 8 months, from about 8 months to about 10 months, from about 10 months to about 12 months), from about 12 months to about 18 months (e.g., from about 12 months to about 14 months, from about 14 months to about 16 months, or from about 16 months to about 18 months), or from about 18 months to about 24 months (e.g., from about 18 months to about 20 months, from about 20 months to about 22 months, or from about 22 months to about 24 months).

In certain embodiments, the method comprises administering one or more cells transduced with a recombinant vector comprising a presently disclosed expression cassette as described above. The vector copy number of the recombinant vector in the cells that provide for the therapeutically relevant level of hemoglobin (e.g., 9-10 g/dL) in the subject is from about 0.5 to about 2, from about 0.5 to about 1, or from about 1 to about 2 vector copy number per cell. In certain embodiments, the vector copy number of the presently disclosed vector is about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, or about 2.0 vector copy number per cell.

In certain embodiments, the subject lacks a human leukocyte antigen (HLA)-matched donor. In certain embodiments, the transduced cell is from the same subject. In certain embodiments, the transduced cell is from bone marrow of the same subject. Thus, administration of the transduced cells do not incur the risk of graft-versus host disease in the subject. The method does not require immune suppression to prevent graft rejection, e.g., the method does not comprise administering an immunosuppressive agent to the subject.

The present disclosed subject matter also provides a method of increasing the proportion of red blood cells or erythrocytes compared to white blood cells or leukocytes in a subject. In various non-limiting embodiments, the method comprises administering an effective amount of a presently disclosed transduced cell or a population of the presently disclosed transduced cells (e.g., HSCs, embryonic stem cells, or iPSCs) to the subject, wherein the proportion of red blood cell progeny cells of the hematopoietic stem cells are increased compared to white blood cell progeny cells of the hematopoietic stem cells in the subject.

An important advantage provided by the expression cassette, vectors and other delivery systems (e.g., nucleases and CRISPR-Cas systems), compositions, and methods of the presently disclosed subject is the high efficacy of globin gene therapy that can be achieved by administering populations of cells comprising lower percentages of transduced cells compared to existing methods.

This provides important safety advantages associated with reduced chances of deleterious mutation, transformation, or oncogene activation of cellular genes in transduced cells. The transduced cells can be administered as part of a bone marrow or cord blood transplant in an individual that has or has not undergone bone marrow ablative therapy.

One consideration concerning the therapeutic use of the presently disclosed cells transduced with the expression cassette described herein (“transduced cells”) is the quantity of cells necessary to achieve an optimal effect. The quantity of transduced cells to be administered will vary for the subject being treated. In certain embodiments, from about 1×10⁴ to about 1×10⁵ cells/kg, from about 1×10⁵ to about 1×10⁶ cells/kg, from about 1×10⁶ to about 1×10⁷ cells/kg, from about 1×10⁷ to about 1×10⁸ cells/kg, from about 1×10⁸ to about 1×10⁹ cells/kg, or from about 1×10⁹ to about 1×10¹⁰ cells/kg of the presently disclosed transduced cells are administered to a subject. More effective cells may be administered in even smaller numbers. In certain embodiments, at least about 1×10⁸ cells/kg, at least about 2×10⁸ cells/kg, at least about 3×10⁸ cells/kg, at least about 4×10⁸ cells/kg, or at least about 5×10⁸ cells/kg of the presently disclosed transduced cells are administered to a subject. The precise determination of what would be considered an effective dose may be based on factors individual to each subject, including their size, age, sex, weight, and condition of the particular subject. Dosages can be readily ascertained by those skilled in the art from this disclosure and the knowledge in the art.

In certain embodiments, the expression cassettes, vectors and other delivery systems (nucleases and CRISPR-Cas systems), compositions, and methods of the presently disclosed subject matter offer improved methods of gene therapy using ex vivo gene therapy and autologous transplantation. Transplantation of cells transduced with the expression cassette or into subjects having a hemoglobinopathy results in long-term correction of the disease.

One or more presently disclosed transduced cells can be administered by any methods known in the art, including, but not limited to, parenteral administration (e.g., intramuscular administration, intravenous administration, subcutaneous administration, or intraperitoneal administration), spinal administration, and epidermal administration. In certain embodiments, one or more transduced cells are delivered to a subject intravenously. One or more presently disclosed transduced cells can be administered by injection, infusion, or implantation. In certain embodiments, one or more transduced cells are administered by injection. In certain embodiments, one or more transduced cells are administered by intravenous injection.

The subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

### VII. Kits

The presently disclosed subject matter provides kits for the treatment or prevention of a hemoglobinopathy. In certain embodiments, the kit comprises a therapeutic or prophylactic composition containing an effective amount of a cell transduced with the presently disclosed expression cassette in unit dosage form. In certain embodiments, the kit comprises one or more expression cassettes disclosed herein. In certain embodiments, the kit comprises one or more vectors comprising an expression cassette disclosed herein. In certain embodiments, the kit comprises a sterile container, which can be a box, an ampule, a bottle, a vial, a tube, a bag, a pouch, a blister-pack, or other suitable container forms known in the art. Such containers can be made of plastic, glass, laminated paper, metal foil, or other materials suitable for holding medicaments.

If desired, the transduced cell is provided together with instructions for administering the cell to a subject having or at risk of developing a hemoglobinopathy. The instructions will generally include information about the use of the composition for the treatment or prevention of a hemoglobinopathy. In certain embodiments, the instructions include at least one of the following: description of the therapeutic agent; dosage schedule and administration for treatment or prevention of a hemoglobinopathy or symptoms thereof; precautions; warnings; indications; counter-indications; overdosage information; adverse reactions; animal pharmacology; clinical studies; and/or references.

Alternatively or additionally, the kit can include instructions for transducing a cell with the one or more expression cassettes and/or vectors comprising such expression cassettes. The instructions may be printed directly on the container (when present), or as a label applied to the container, or as a separate sheet, pamphlet, card, or folder supplied in or with the container.

### Examples

The practice of the presently disclosed subject matter employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the presently disclosed subject matter, and, as such, may be considered in making and practicing the presently disclosed subject matter. Particularly useful techniques for particular embodiments will be discussed in the sections that follow.

The following examples are put forth so as to provide those of ordinary skill in the art with a complete disclosure and description of how to make and use the expression cassettes, vectors, delivery systems, and therapeutic methods of the presently disclosed subject matter, and are not intended to limit the scope of what the inventors regard as their invention.

### Example 1: Discovery of Novel Insulators

The problems created by insertional mutagenesis of viral vectors are widely known (Nienhuis (2013), Baum et al. (2006), Nienhuis et al. (2006)) as is the evidence that the risks of genotoxicity can be reduced by the use of chromatin insulators (Arumugam et al. (2007), Emery (2011), Evans-Galea et al. (2007), Rivella et al. (2000), Emery et al. (2000), Emery et al. (2002), Yannaki et al. (2002), Hino et al. (2004), Ramezani et al. (2003), Ramezani et al. (2008)). Approaches allowing the efficient identification of enhancer blocking insulators in the human genome have been developed. These new insulators are short, on the average 150 bp, and they do not affect adversely the titers of viral vectors and they are several times more powerful than the insulator cHS4. Genomic approaches were used to discover the most powerful enhancer blocker and barrier insulators of the human genome. For gene therapy of the hemoglobinopathies, powerful enhancers are required to achieve therapeutic levels of globin gene expression. Powerful insulators may thus provide one means to protect the genomic environment from the powerful enhancers of the integrating vectors.

Several studies have demonstrated the ability of the cHS4 insulator to reduce position-effect silencing of gammaretroviral vectors (Evans-Galea et al. (2007), Rivella et al. (2000), Emery et al. (2000), Emery et al. (2002), Yannaki et al. (2002), Hino et al. (2004), Ramezani et al. (2006), Yao et al. (2003), Nishino et al. (2006), Aker et al. (2007), Li and Emery (2008)), and lentiviral vectors (Evans-Galea et al. (2007), Ramezani et al. (2003), Puthenveetil et al. (2004), Arumugam et al. (2007), Bank et al. (2005), Aker et al. (2007), Ma et al. (2003), Chang et al. (2005), Pluta et al. (2005)). Those studies that were appropriately designed demonstrated that inclusion of the 1.2 kb version of the cHS4 insulator increased the likelihood and/or consistency of vector transgene expression in at least some settings (Arumugam et al. (2007), Evans-Galea et al. (2007), Emery et al. (2002), Yannaki et al. (2002), Hino et al. (2004), Ramezani et al. (2006), Aker et al. (2007), Li and Emery (2008), Pluta et al. (2005), Jakobsson et al. (2004)). Nevertheless, the degree of protection afforded by the cHS4 insulator is far from complete. In addition, the inclusion of the 1.2 Kb cHS4 can adversely affect vector titers while the smallest cHS4 core has been proven ineffective (Aker et al. (2007), Jakobsson et al. (2004)).

Effects on genotoxicity were tested using an in vivo assay based on quantitation of tumor formation in mice. Vectors insulated by insulator A1 decreased tumor formation induced by random vector integration in hematopoietic chimeras compared to mice that received uninsulated or cHS4-insulated controls.

To assess effects on vector titers, insulator A1 was introduced into the double-copy region of a third-generation lentiviral vector expressing GFP from a constitutive package promoter, and the viral titers and GFP expression were measured. Insulator A1 did not affect adversely vector GFP expression.

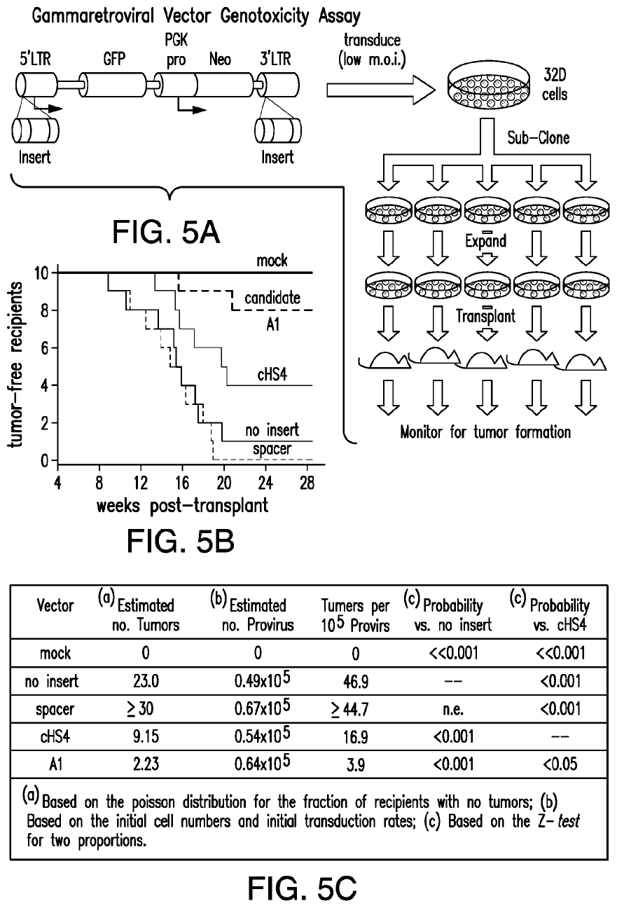

In the in vivo genotoxicity assay, a cell line transduced with gammaretroviral vectors produced tumors after transplantation in mice and allowed quantitation of genotoxic effects by measuring rates of tumor free survival. Effects of an insulator on genotoxicity were quantitated by the number of tumors formed in the mice and the rates of tumor free survival. Insulator A1 was inserted in the proximal portion of the 3′ LTR, from which it is copied into the 5′ LTR during reverse transcription and vector integration. The resulting topology places copies of the insulator between the genomic regions located 5′ and 3′ of the integrated provirus and enhancer activity from the 5′ viral LTR and internal Pgk promoter, but does not contain the enhancer in the 3′ LTR. This can decrease genotoxicity thus resulting in decreased tumor formation and increased survival of the animals. Gamma-retroviral reporter vectors flanked with insulator A1 or control regions were used to transduce the growth factor-dependent cell line 32D, and 10 independent sub-pools for each vector were transplanted into syngeneic C3H/HeJ mice. All 10 mice transplanted with mock-transduced cells remained free of 32D cell-derived tumors, while nearly all mice transplanted with 32D cells transduced with vectors containing no inserts or a 790 bp neutral spacer developed tumors within a median of 16 weeks (FIG. 5B). Flanking this vector with the cHS4 insulator delayed the onset of tumor formation by several weeks, and reduced the frequency of animals that developed tumors to 6 of 10. In contrast, only two of 10 animals developed tumors following transplantation with 32D cells transduced with the vector flanked with insulator A1 (FIG. 5B). The frequency of animals with tumors and the number of vector transduction events in the original sub-pools suggested that flanking the vector with insulator A1 reduced the overall rate of tumor formation 12-fold, from 46.9 tumors per 10⁵ provirus to 3.9 tumors per 10⁵ provirus (FIG. 5C). In comparison, the cHS4 insulator reduced the overall rate of tumor formation 2.8-fold (to 16.9 tumors per 10⁵ provirus), while the neutral spacer had no statistically discernable effect on the rate of tumor formation. These results indicate that the discovered enhancer blocking insulators can decrease substantially the risks of insertional mutagenesis and genotoxicity.

### Example 2: Characterization of Globin Vectors Comprising At Least One Insulator

A presently disclosed expression cassette (designated as “Expression Cassette 1”; as shown in FIG. 1), which comprises insulator A1, and a human β^{A}-globin gene encoding a threonine to glutamine mutation at codon 87 (β^{A-T87Q}) operably linked to a β-globin LCR comprising a HS2 region having the nucleotide sequence set forth in SEQ ID NO:9, a HS3 region having the nucleotide sequence set forth in SEQ ID NO:5, and a HS4 region having the nucleotide sequence set forth in SEQ ID NO:7, was generated. The rationale for using the variant β chain (β^{A}) is to facilitate the detection of the vector-encoded β-globin gene, distinguishing it from endogenous or transfused beta chains. The glutamine (GLN) residue at position 87 in the γ-globin chain augments the anti-sickling activity of the gamma chain relative to the β chain, while preserving adult oxygen-binding characteristics of the β chain (Nagel et al. (1979)). In Vector 1, a point mutation altering codon 87 (β^{A}-T870, or β87) replaces the normal threonine with glutamine and augments anti-sickling activity of the vector-encoded β chain. This β87 chain has been safely used in a patient with HbE-thalassemia (Cavazzana-Calvo et al. (2010))

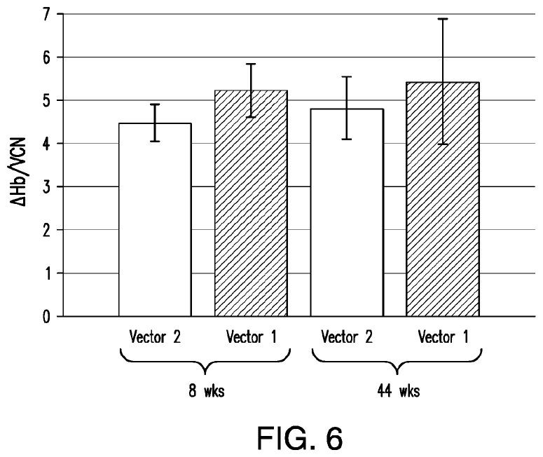

Expression cassette 1 was incorporated or introduced to a lentivirus vector (designated as “Vector 1”). Vector 1 was introduced in bone marrow cells of C57BL/6-Hbb th3/+ mice and transplanted to syngeneic lethally irradiated recipients as previously described (May et al. (2000), May et al. (2002), Lisowski et al. (2007)). The vector titer of VI was comparable to that of a lentivirus vector comprising an expression cassette lacking insulator A1. The β-globin expression of Vector 1 was compared to that of a lentivirus vector (designated as “Vector 2”) comprising an expression cassette that lacks an insulator and comprises a wild human β-globin gene operably linked to a β-globin LCR comprising a HS2 region having the nucleotide sequence set forth in SEQ ID NO:9, a HS3 region having the nucleotide sequence set forth in SEQ ID NO:5, and a HS4 region having the nucleotide sequence set forth in SEQ ID NO:6. In comparison to Vector 2, β-globin expression of Vector 1 normalized to vector copy was equivalent or slightly increased, suggesting an added benefit for in vivo expression provided by the flanking barrier elements, as shown in FIG. 6.

### Example 3: Evaluation of Enhancer Activity in Non-Erythroid K562 Cells

The enhancer activity of HS2 was evaluated in Non-erythroid K562 Cells. As shown in FIG. 7, GFP expression in K562 cells transduced with vectors driven by a minimal promoter linked to no enhancer (“Empty”, HS2, HS3-4, HS2-3-4 or the runx1 enhancer used as positive control (“RUNX1”). Background expression was on the order or 0.01% (“empty), but increased over 10-fold with HS2-3-4 (“Lcr9”, 0.17%). This enhancement was mostly due to HS2 (0.15%) but not HS3-4 (0.05%). All cell lines were comparably transduced (mean vector copy number 2.5). The results support that HS2 but not HS3-HS4 may pose an oncogenic risk in non-erythroid hematopoietic stem and progenitor cells.

### Example 4: Novel Erythroid-Specific Enhancers

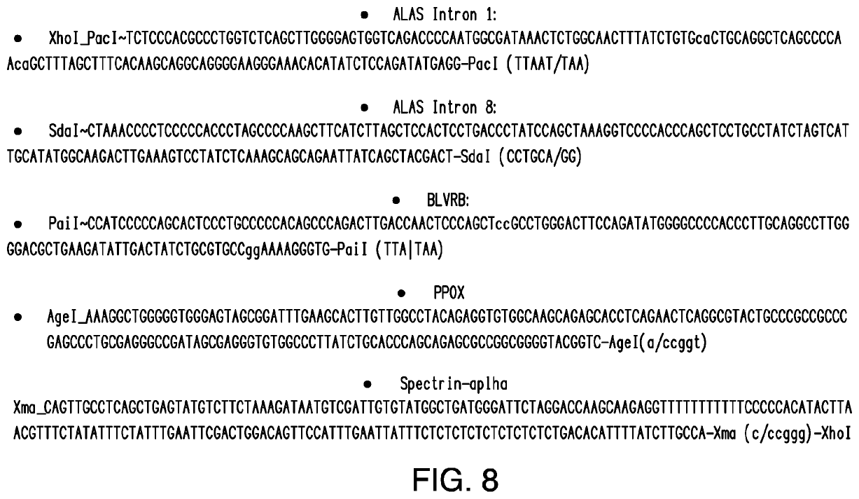

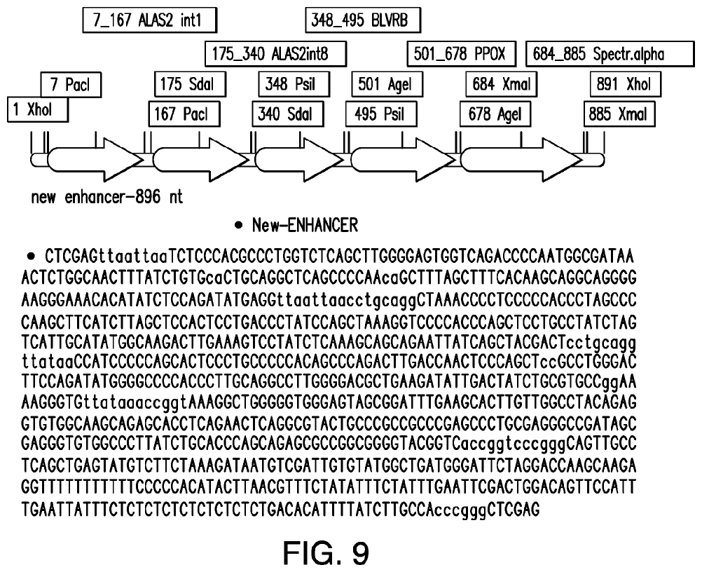

As shown in FIGS. 8 and 9, five erythroid-specific enhancers were substituted for HS2: ALAS Intron 1, ALAS Intron 8, BLVRB, PPOX, and Spectrin-alpha. The inventors have shown that all these five enhancers are powerful enhancers, and lack enhancer activity in non-erythroid tissues, and do not reduce the vector titer.

### Example 5: Increasing Globin Lentiviral Vector Production Through 3′ LTR Modifications

An essential feature of therapeutic globin vectors is to achieve a high titer, sufficient for effective transduction of patient cells. By virtue of their large cargo, comprising a gene, promoter, enhancers and/or LCR elements, globin lentiviral vectors inherently have low titer, complicating their manufacture and limiting their clinical use. This problem is further compounded by the incorporation of additional genomic elements such as an insulator, which further increase the size of the vector.

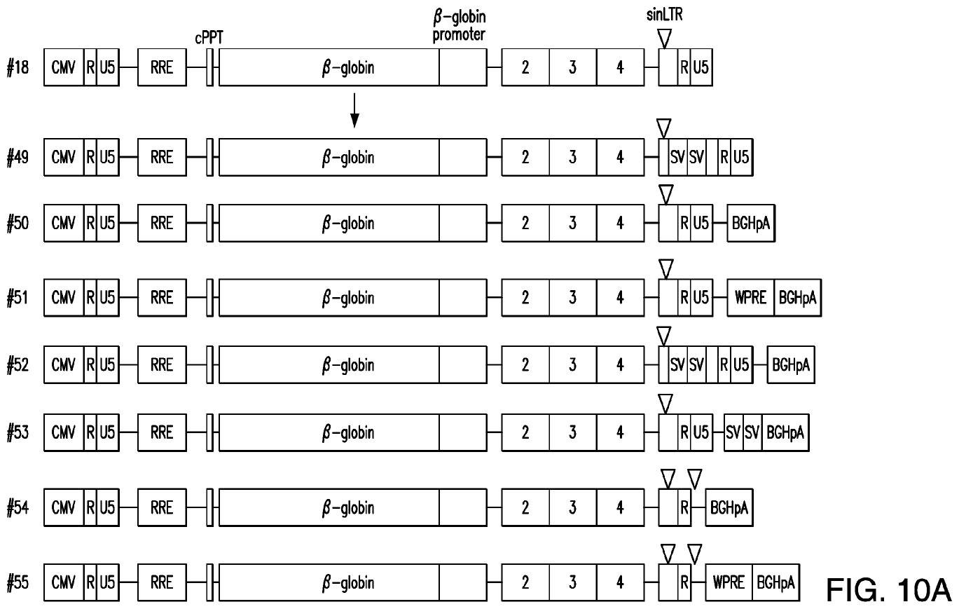

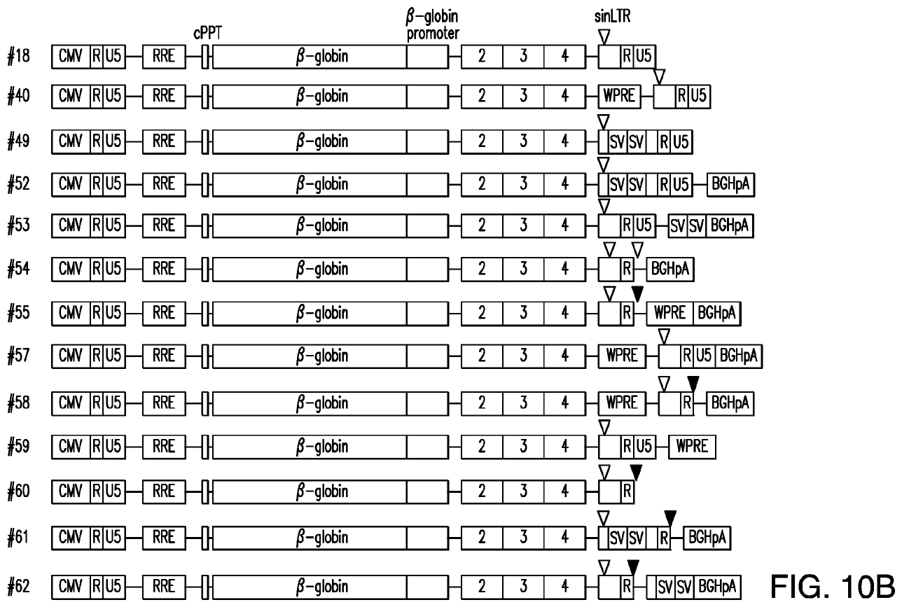

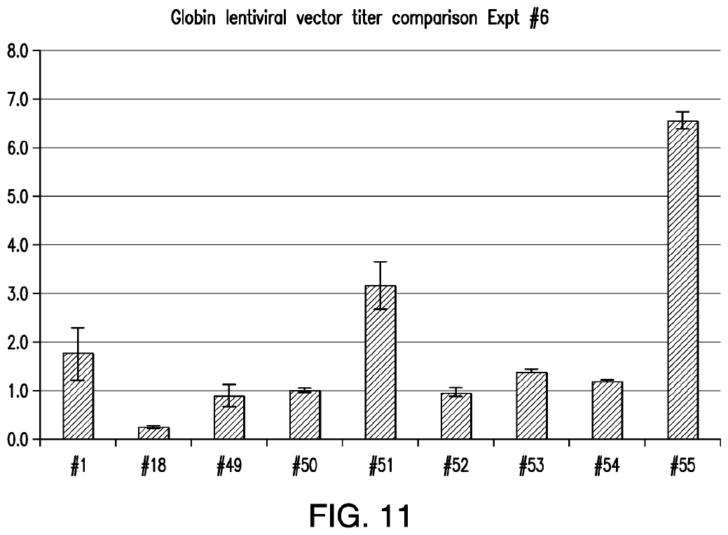

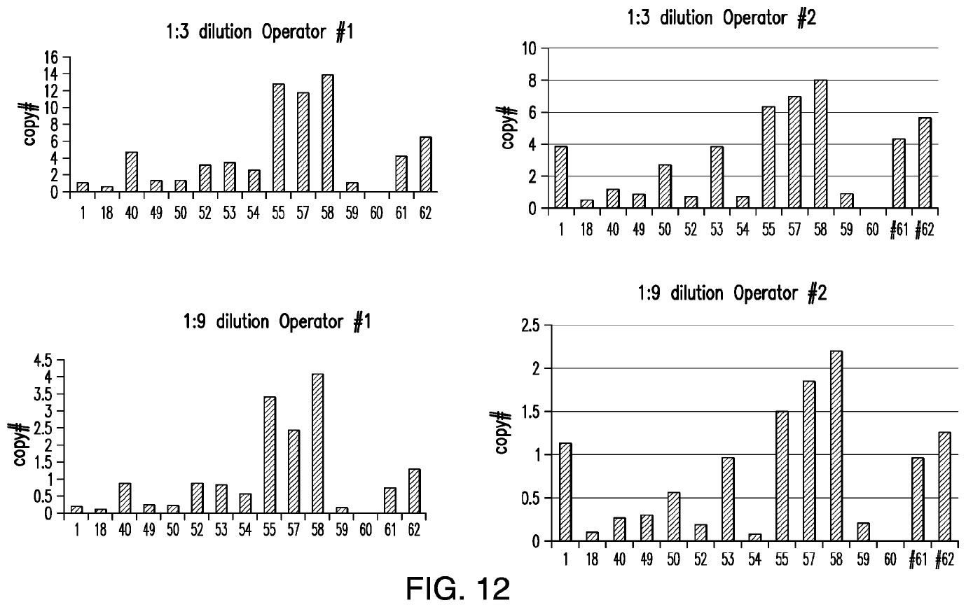

The inventors explored different modifications of the 3′ long terminal repeat (LTR) of globin vectors to increase the titer of globin vectors. Over 62 variations were evaluated, numbered 1 through 62, modeled on a lentivirus vector comprising a human β-globin gene operably linked to a β-globin LCR comprising a HS2 region having the nucleotide sequence set forth in SEQ ID NO:9, a HS3 region having the nucleotide sequence set forth in SEQ ID NO:5, and a HS4 region having the nucleotide sequence set forth in SEQ ID NO:7. In other words, all of Vectors #1 through Vector 62 comprise a β-globin LCR comprising a HS2 region having the nucleotide sequence set forth in SEQ ID NO:9, a HS3 region having the nucleotide sequence set forth in SEQ ID NO:5, and a HS4 region having the nucleotide sequence set forth in SEQ ID NO:7. Vector #18 served as a baseline, comprising a standard U3 deletion in the 3′LTR. Vector #1 (not depicted) comprised a full, i.e., wild-type LTR, which cannot be used clinically. Modifications to the 3′LTR are depicted in FIGS. 10A and 10B, and their titers shown in FIGS. 11 and 12 (the Y axis shows the vector copy number of vector stocks manufactured and tested under strictly identical conditions). Titrations were measured in triple replicas, performed in parallel by two operators, and repeated in multiple experiments.

As shown in FIGS. 11 and 12, Vector #55 repeatedly showed a higher titer. This vector comprises a Woodchuck hepatitis post-regulatory element (WPRE) and a bovine growth hormone polyadenylation signal 3′ to the R region in the 3′ LTR. The WPRE element is therefore not transferred to the transduced cells.

### Example 6: Generation of Globin Vectors

### Methods and Materials

### Vector Production

Various lentiviral vectors were produced, including TNS9.B87.A1, SNS22.2.B87.A1, SNS23.2.B87.A1, and SNS24.2.B87.A1. The nucleotide sequences of TNS9.B87.A1, SNS22.2.B87.A1, SNS23.2.B87.A1, SNS24.2.B87.A1, SNS26.B87.A1 and SNS27.2.B87.A1 are set forth in SEQ ID NO: 37, SEQ ID NO: 38, SEQ ID NO: 39, SEQ ID NO: 40, SEQ ID NO: 76, and SEQ ID NO: 77, respectively.

[SEQ ID NO: 37]
attggccattgcatacgttgtatccatatcataatatgtacatttatattggctcatgtccaacattaccgccatgttga
cattgattattgactagttattaatagtaatcaattacggggtcattagttcatagcccatatatggagttccgcgttac
ataacttacggtaaatggcccgcctggctgaccgcccaacgacccccgcccattgacgtcaataatgacgtatgttccca
tagtaacgccaatagggactttccattgacgtcaatgggtggagtatttacggtaaactgcccacttggcagtacatcaa
gtgtatcatatgccaagtacgccccctattgacgtcaatgacggtaaatggcccgcctggcattatgcccagtacatgac
cttatgggactttcctacttggcagtacatctacgtattagtcatcgctattaccatggtgatgcggttttggcagtaca
tcaatgggcgtggatagcggtttgactcacggggatttccaagtctccaccccattgacgtcaatgggagtttgttttgg
caccaaaatcaacgggactttccaaaatgtcgtaacaactccgccccattgacgcaaatgggcggtaggcgtgtacggtg
ggaggtctatataagcagagctcgtttagtgaaccggggtctctctggttagaccagatctgagcctgggagctctctgg
ctaactagggaacccactgcttaagcctcaataaagcttgccttgagtgcttcaagtagtgtgtgcccgtctgttgtgtg
actctggtaactagagatccctcagacccttttagtcagtgtggaaaatctctagcagtggcgcccgaacagggacttga
aagcgaaagggaaaccagaggagctctctcgacgcaggactcggcttgctgaagcgcgcacggcaagaggcgaggggcgg
cgactggtgagtacgccaaaaattttgactagcggaggctagaaggagagagacgggtgcgagagcgtcagtattaagcg
ggggtgaataagatcgcgatgggaaaaaattcggttaaggccagggggaaagaaaaaatataaattaaaacatatagtat
gggcaagcagggagctagaacgattcgcagttaatcctggcctgttagaaacatcagaaggctgtagacaaatactggga
cagctacaaccatcccttcagacaggatcagaagaacttagatcattatataatacagtagcaaccctctattgtgtgca
tcaaaggatagagataaaagacaccaaggaagctttagacaagatagaggaagagcaaaacaaaagtaagaccaccgcac
agcaagcggccgctgatcttcagacctggaggaggagatatgagggacaattggagaagtgaattatataaatataaagt
agtaaaaattgaaccattaggagtagcacccaccaaggcaaagagaagagtggtgcagagagaaaaaagagcagtgggaa
taggagctttgttccttgggttcttgggagcagcaggaagcactatgggcgcagcgtcaatgacgctgacggtacaggcc
agacaattattgtctggtatagtgcagcagcagaacaatttgctgagggctattgaggcgcaacagcatctgttgcaact
cacagtctggggcatcaagcagctccaggcaagaatcctggctgtggaaagatacctaaaggatcaacagctcctgggga
tttggggttgctctggaaaactcatttgcaccactgctgtgccttggaatgctagttggagtaataaatctctggaacag
atttggaatcacacgacctggatggagtgggacagagaaattaacaattacacaagcttaatacactccttaattgaaga
atcgcaaaaccagcaagaaaagaatgaacaagaattattggaattagataaatgggcaagtttgtggaattggtttaaca
taacaaattggctgtggtatataaaattattcataatgatagtaggaggcttggtaggtttaagaatagtttttgctgta
ctttctatagtgaatagagttaggcagggatattcaccattatcgtttcagacccacctcccaaccccgaggggacccga
caggcccgaaggaatagaagaagaaggtggagagagagacagagacagatccattcgattagtgaacggatctcgacggt
atcgttttaaaagaaaaggggggattggggggtacagtgcaggggaaagaatagtagacataatagcaacagacatacaa
actaaagaattacaaaaacaaattacaaaaattcaaaattttatcggcgtgttgggggtggaccatcctctaggtattga
ataagaaaaatgaagttaaggtggttgatggtaacactatgctaataactgcagagccagaagcaccataagggacatga
taagggagccagcagacctctgatctcttcctgaatgctaatcttaaacatcctgaggaagaatgggacttccatttggg
gtgggcctatgatagggtaataagacagtagtgaatatcaagctacaaaaagccccctttcaaattcttctcagtcctaa
cttttcatactaagcccagtccttccaaagcagactgtgaaagagtgatagttccgggagactagcactgcagattccgg
gtcactgtgagtgggggaggcagggaagaagggctcacaggacagtcaaaccatgccccctgtttttccttcttcaagta
gacctctataagacaacagagacaactaaggctgagtggccaggcgaggagaaaccatctcgccgtaaaacatggaagga
acacttcaggggaaaggtggtatctctaagcaagagaactgagtggagtcaaggctgagagatgcaggataagcaaatgg
gtagtgaaaagacattcatgaggacagctaaaacaataagtaatgtaaaatacagcatagcaaaactttaacctccaaat
caagcctctacttgaatccttttctgagggatgaataaggcataggcatcaggggctgttgccaatgtgcattagctgtt
tgcagcctcaccttctttcatggagtttaagatatagtgtattttcccaaggtttgaactagctcttcatttctttatgt
tttaaatgcactgacctcccacattccctttttagtaaaatattcagaaataatttaaatacatcattgcaatgaaaata
aatgttttttattaggcagaatccagatgctcaaggcccttcataatatcccccagtttagtagttggacttagggaaca
aaggaacctttaatagaaattggacagcaagaaagcgagcttagtgatacttgtgggccagggcattagccacaccagcc
accactttctgataggcagcctgcactggtggggtgAATTCTTTGCCAAAGTGATGGGCCAGCACACAGACCAGCACGTT
GCCCAGGAGCTGTGGGAGGAAGATAAGAGGTATGAACATGATTAGCAAAAGGGCCTAGCTTGGACTCAGAATAATCCAGC
CTTATCCCAACCATAAAATAAAAGCAGAATGGTAGCTGGATTGTAGCTGCTATTAGCAATATGAAACCTCTTACATCAGT
TACAATTTATATGCAGAAATATTTATATGCAGAAATATTGCTATTGCCTTAACCCAGAAATTATCACTGTTATTCTTTAG
AATGGTGCAAAGAGGCATGATACATTGTATCATTATTGCCCTGAAAGAAAGAGATTAGGGAAAGTATTAGAAATAAGATA
AACAAAAAAGTATATTAAAAGAAGAAAGCATTTTTTAAAATTACAAATGCAAAATTACCCTGATTTGGTCAATATGTGTA
CCCTGTTACTTCTCCCCTTCCTATGACATGAACTTAACCATAGAAAAGAAGGGGAAAGAAAACATCAAGGGTCCCATAGA
CTCACCCTGAAGTTCTCAGGATCCACGTGCAGCTTGTCACAGTGCAGCTCACTCAGctgGGCAAAGGTGCCCTTGAGGTT
GTCCAGGTGAGCCAGGCCATCACTAAAGGCACCGAGCACTTTCTTGCCATGAGCCTTCACCTTAGGGTTGCCCATAACAG
CATCAGGAGTGGACAGATCCCCAAAGGACTCAAAGAACCTCTGGGTCCAAGGGTAGACCACCAGCAGCCTAAGGGTGGGA
AAATAGACCAATAGGCAGAGAGAGTCAGTGCCTATCAGAAACCCAAGAGTCTTCTCTGTCTCCACATGCCCAGTTTCTAT
TGGTCTCCTTAAACCTGTCTTGTAACCTTGATACCAACCTGCCCAGGGCCTCACCACCAACTTCATCCACGTTCACCTTG
CCCCACAGGGCAGTAACGGCAGACTTCTCCTCAGGAGTCAGGTGCACCATGGTGTCTGTTTGAGGTTGCTAGTGAACACA
GTTGTGTCAGAAGCAAATGTAAGCAATAGATGGCTCTGCCCTGACTTTTATGCCCAGCCCTGGCTCCTGCCCTCCCTGCT
CCTGGGAGTAGATTGGCCAACCctagggtgtggctccacagggtgaggtctaagtgatgacagccgtacctgtccttggc
tcttctggcactggcttaggagttggacttcaaaccctcagccctccctctaagatatatctcttggccccataccatca
gtacaaattgctactaaaaacatcctcctttgcaagtgtatttacgtaatatttggaatcacagcttggtaagcatattg
aagatcgttttcccaattttcttattacacaaataagaaattgatgcactaaaagtggaagagttttgtctaccataatt
cagctttgggatatgtagatggatctcttcctgcgtctccagaatatgcaaaatacttacaggacagaatggatgaaaac
tctacctcagttctaagcatatcttctccttatttggattaaaaccttctggtaagaaaagaaaaaaaatatatatatat
atgtgtatatatacacacatacatatacatatatatgcattcatttgttgttgtttttcttaatttgctcatggtatatg
tgtatatatatatatatatattcaggaaataatatattctagaatatgtcacattctgtctcaggcatccattttcttta
tgatgccgtttgaggtggagttttagtcaggtggtcagcttctccttttttttgccatctgccctgtaagcatcctgctg
gggacccagataggagtcatcactctaggctgagaacatctgggcacacaccctaagcctcagcatgactcatcatgact
cagcattgctgtgcttgagccagaaggtttgcttagaaggttacacagaaccagaaggcgggggtggggcactgaccccg
acaggggcctggccagaactgctcatgcttggactatgggaggtcactaatggagacacacagaaatgtaacaggaacta
aggaaaaactgaagcttatttaatcagagatgagatgctggaagggatagagggagctgagcttgtaaaaagtatagtaa
tcattcagcaaatggttttgaagcacctgctggatgctaaacactattttcagtgcttgaatcataaataagaataaaac
atgtatcttattccccacaagagtccaagtaaaaaataacagttaattataatgtgctctgtcccccaggctggagtgca
gtggcacgatctcagctcactgcaacctccgcctcccgggttcaagcaattctcctgcctcagccaccctaatagctggg
attacaggtgcacaccaccatgccaggctaatttttgtactttttgtagaggcagggtatcaccatgttgtccaagatgg
tcttgaactcctgagctccaagcagtccacccacctcagcctcccaaagtgctatctgcggccgcctatctgtaccacta
gtctcgagaagctttcattaaaaaaagtctaaccagctgcattcgactttgactgcagcagctggttagaaggttctact
ggaggagggtcccagcccattgctaaattaacatcaggctctgagactggcagtatatctctaacagtggttgatgctat
cttctggaacttgcctgctacattgagaccactgacccatacataggaagcccatagctctgtcctgaactgttaggcca
ctggtccagagagtgtgcatctcctttgatcctcataataaccctatgagatagacacaattattactcttactttatag
atgatgatcctgaaaacataggagtcaaggcacttgcccctagctgggggtataggggagcagtcccatgtagtagtaga
atgaaaaatgctgctatgctgtgcctcccccacctttcccatgtctgccctctactcatggtctatctctcctggctcct
gggagtcatggactccacccagcaccaccaacctgacctaaccacctatctgagcctgccagcctataacccatctgggc
cctgatagctggtggccagccctgaccccaccccaccctccctggaacctctgatagacacatctggcacaccagctcgc
aaagtcaccgtgagggtcttgtgtttgctgagtcaaaattccttgaaatccaagtccttagagactcctgctcccaaatt
tacagtcatagacttcttcatggctgtctcctttatccacagaatgattcctttgcttcattgccccatccatctgatcc
tcctcatcagtgcagcacagggcccatgagcagtagctgcagagtctcacataggtctggcactgcctctgacatgtccg
accttaggcaaatgcttgactcttctgagctcagtcttgtcatggcaaaataaagataataatagtgtttttttatggag
ttagcgtgaggatggaaaacaatagcaaaattgattagactataaaaggtctcaacaaatagtagtagattttatcatcc
attaatccttccctctcctctcttactcatcccatcacgtatgcctcttaattttcccttacctataataagagttattc
ctcttattatattcttcttatagtgattctggatattaaagtgggaatgaggggcaggccactaacgaagaagatgtttc
tcaaagaagccattctccccacatagatcatctcagcagggttcaggaagataaaggaggatcaaggtcgaaggtaggaa
ctaaggaagaacactgggcaagtggatcctgagccccttttcctctaactgaaagaaggaaaaaaaaaatggaacccaaa
atattctacatagtttccatgtcacagccagggctgggcagtctcctgttatttcttttaaaataaatatatcatttaaa
tgcataaataagcaaaccctgctcgggaatgggagggagagtctctggagtccaccccttctcggccctggctctgcaga
tagtgctatcaaagccctgacagagccctgcccattgctgggccttggagtgagtcagcctagtagagaggcagggcaag
ccatctcatagctgctgagtgggagagagaaaagggctcattgtctataaactcaggtcatggctattcttattctcaca
ctaagaaaaagaatgagatgtctacatataccctgcgtcccctcttgtgtactggggcccccaagagctctctaaaagtg
atggcaaagtcattgcgctagatgccatcccatctattataaacctgcatttgtctccacacaccagtcatggacaataa
ccctcctcccaggtccacgtgcttgtctttgtataatactcaagtaatttcggaaaatgtattctttcaatcttgttctg
ttattcctgtttcaatggcttagtagaaaaagtacatacttgttttcccataaattgacaatagacaatttcacatcaat
gtctatatgggtcgttgtgtttgctgtgtttgcaaaaactcacaataactttatattgttactactctaagaaagttaca
acatggtgaatacaagagaaagctattacaagtccagaaaataaaagttatcatcttgaggcctcagctttctaggaata
atatcaatattacaaaatttaatctaacaattatgaacagcaatgagataatatgtacaaagtacccagacctatgtggt
agagcatcaaggaagcgcattgcggagcagttttttgtttgtttgtttttgtattctgtttcgtgaggcaaggtttcact
ctgctgtccaggctggagtgcagtggcaagatcatgtctcactgcagccttgacacgcgtcgacggtaccgttaacgatc
ttagccactttttaaaagaaaaggggggactggaagggctaattcactcccaacgaagacaagatatcctgCTAGTCCTT
CCTTTCTAAATGACGAGAGAGACAGAAGAATTCTTCAAGGTTAGTGTGTCCAGCATGCAACCTTTCCTTCCTGGATGAGC
ATCCCTGGAGTAGGAGAGCCAGCCTGCCTCCTGCGCTGGCACAGAGCCCGGTTCCCTAGACAACTGCCTCTCCAAATCTG
ATGTCCAGCGCCACCTGGTGTCCACATCAAGCAGACACAATTAATAGTCAACCTGTTCAGGAAAACTGTGAGGGGGAAAA
AAAAGAAAGAGGATTTATGAAGGGAAAAGAAAGTTTAGAGGATATGCCACGATTGGctagcagctgctttttgcctgtac
tgggtctctctggttagaccagatctgagcctgggagctctctggctaactagggaacccactgcttaagcctcaataaa
gcttgccttgagtgcttcatccggAATCAACCTCTGGATTACAAAATTTGTGAAAGATTGACTGGTATTCTTAACTATGT
TGCTCCTTTTACGCTATGTGGATACGCTGCTTTAATGCCTTTGTATCATGCTATTGCTTCCCGTATGGCTTTCATTTTCT
CCTCCTTGTATAAATCCTGGTTGCTGTCTCTTTATGAGGAGTTGTGGCCCGTTGTCAGGCAACGTGGCGTGGTGTGCACT
GTGTTTGCTGACGCAACCCCCACTGGTTGGGGCATTGCCACCACCTGTCAGCTCCTTTCCGGGACTTTCGCTTTCCCCCT
CCCTATTGCCACGGCGGAACTCATCGCCGCCTGCCTTGCCCGCTGCTGGACAGGGGCTCGGCTGTTGGGCACTGACAATT
CCGTGGTGTTGTCGGGGAAGCTGACGTCCTTTCCATGGCTGCTCGCCTGTGTTGCCACCTGGATTCTGCGCGGGACGTCC
TTCTGCTACGTCCCTTCGGCCCTCAATCCAGCGGACCTTCCTTCCCGCGGCCTGCTGCCGGCTCTGCGGCCTCTTCCGCG
TCTTCGCCTTCGCCCTCAGACGAGTCGGATCTCCCTTTGGGCCGCCTCCCCGCCTGtccggtAGCTTGCCAGCCTCGACT
GTGCCTTCTAGTTGCCAGCCGTCTGTTGTTTGCCCCTCCCCCGTGCCTTCCTTGACCCTGGAAGGTGCCACTCCCACTGT
CCTTTCCTAATAAAATGAGGAAATTGCATCGCATTGTCTGAGTAGGTGTCATTCTATTCTGGGGGGTGGGGTGGGGCAGG
ACAGCAAGGGGGAGGATTGGGAAGACAATAGCAGGCATGcaagcttggcgtaatcatggtcatagctgtttcctgtgtga
aattgttatccgctcacaattccacacaacatacgagccggaagcataaagtgtaaagcctggggtgcctaatgagtgag
ctaactcacattaattgcgttgcgctcactgcccgctttccagtcgggaaacctgtcgtgccagctgcattaatgaatcg
gccaacgcgcggggagaggcggtttgcgtattgggcgctcttccgcttcctcgctcactgactcgctgcgctcggtcgtt
cggctgcggcgagcggtatcagctcactcaaaggcggtaatacggttatccacagaatcaggggataacgcaggaaagaa
catgtgagcaaaaggccagcaaaaggccaggaaccgtaaaaaggccgcgttgctggcgtttttccataggctccgccccc
ctgacgagcatcacaaaaatcgacgctcaagtcagaggtggcgaaacccgacaggactataaagataccaggcgtttccc
cctggaagctccctcgtgcgctctcctgttccgaccctgccgcttaccggatacctgtccgcctttctcccttcgggaag
cgtggcgctttctcatagctcacgctgtaggtatctcagttcggtgtaggtcgttcgctccaagctgggctgtgtgcacg
aaccccccgttcagcccgaccgctgcgccttatccggtaactatcgtcttgagtccaacccggtaagacacgacttatcg
ccactggcagcagccactggtaacaggattagcagagcgaggtatgtaggcggtgctacagagttcttgaagtggtggcc
taactacggctacactagaagaacagtatttggtatctgcgctctgctgaagccagttaccttcggaaaaagagttggta
gctcttgatccggcaaacaaaccaccgctggtagcggtggtttttttgtttgcaagcagcagattacgcgcagaaaaaaa
ggatctcaagaagatcctttgatcttttctacggggtctgacgctcagtggaacgaaaactcacgttaagggattttggt
catgagattatcaaaaaggatcttcacctagatccttttaaattaaaaatgaagttttaaatcaatctaaagtatatatg
agtaaacttggtctgacagttaccaatgcttaatcagtgaggcacctatctcagcgatctgtctatttcgttcatccata
gttgcctgactccccgtcgtgtagataactacgatacgggagggcttaccatctggccccagtgctgcaatgataccgcg
agacccacgctcaccggctccagatttatcagcaataaaccagccagccggaagggccgagcgcagaagtggtcctgcaa
ctttatccgcctccatccagtctattaattgttgccgggaagctagagtaagtagttcgccagttaatagtttgcgcaac
gttgttgccattgctacaggcatcgtggtgtcacgctcgtcgtttggtatggcttcattcagctccggttcccaacgatc
aaggcgagttacatgatcccccatgttgtgcaaaaaagcggttagctccttcggtcctccgatcgttgtcagaagtaagt
tggccgcagtgttatcactcatggttatggcagcactgcataattctcttactgtcatgccatccgtaagatgcttttct
gtgactggtgagtactcaaccaagtcattctgagaatagtgtatgcggcgaccgagttgctcttgcccggcgtcaatacg
ggataataccgcgccacatagcagaactttaaaagtgctcatcattggaaaacgttcttcggggcaaaactctcaaggat
cttaccgctgttgagatccagttcgatgtaacccactcgtgcacccaactgatcttcagcatcttttactttcaccagcg
tttctgggtgagcaaaaacaggaaggcaaaatgccgcaaaaaagggaataagggcgacacggaaatgttgaatactcata
ctcttcctttttcaatattattgaagcatttatcagggttattgtctcatgagcggatacatatttgaatgtatttagaa
aaataaacaaataggggttccgcgcacatttccccgaaaagtgccacctgacgtctaagaaaccattattatcatgacat
taacctataaaaataggcgtatcacgaggccctttcgtctcgcgcgtttcggtgatgacggtgaaaacctctgacacatg
cagctcccggagacggtcacagcttgtctgtaagcggatgccgggagcagacaagcccgtcagggcgcgtcagcgggtgt
tggcgggtgtcggggctggcttaactatgcggcatcagagcagattgtactgagagtgcacc
[SEQ ID NO: 38]
ATTGGCCATTGCATACGTTGTATCCATATCATAATATGTACATTTATATTGGCTCATGTCCAACATTACCGCCATGTTGA
CATTGATTATTGACTAGTTATTAATAGTAATCAATTACGGGGTCATTAGTTCATAGCCCATATATGGAGTTCCGCGTTAC
ATAACTTACGGTAAATGGCCCGCCTGGCTGACCGCCCAACGACCCCCGCCCATTGACGTCAATAATGACGTATGTTCCCA
TAGTAACGCCAATAGGGACTTTCCATTGACGTCAATGGGTGGAGTATTTACGGTAAACTGCCCACTTGGCAGTACATCAA
GTGTATCATATGCCAAGTACGCCCCCTATTGACGTCAATGACGGTAAATGGCCCGCCTGGCATTATGCCCAGTACATGAC
CTTATGGGACTTTCCTACTTGGCAGTACATCTACGTATTAGTCATCGCTATTACCATGGTGATGCGGTTTTGGCAGTACA
TCAATGGGCGTGGATAGCGGTTTGACTCACGGGGATTTCCAAGTCTCCACCCCATTGACGTCAATGGGAGTTTGTTTTGG
CACCAAAATCAACGGGACTTTCCAAAATGTCGTAACAACTCCGCCCCATTGACGCAAATGGGCGGTAGGCGTGTACGGTG
GGAGGTCTATATAAGCAGAGCTCGTTTAGTGAACCGGGGTCTCTCTGGTTAGACCAGATCTGAGCCTGGGAGCTCTCTGG
CTAACTAGGGAACCCACTGCTTAAGCCTCAATAAAGCTTGCCTTGAGTGCTTCAAGTAGTGTGTGCCCGTCTGTTGTGTG
ACTCTGGTAACTAGAGATCCCTCAGACCCTTTTAGTCAGTGTGGAAAATCTCTAGCAGTGGCGCCCGAACAGGGACTTGA
AAGCGAAAGGGAAACCAGAGGAGCTCTCTCGACGCAGGACTCGGCTTGCTGAAGCGCGCACGGCAAGAGGCGAGGGGCGG
CGACTGGTGAGTACGCCAAAAATTTTGACTAGCGGAGGCTAGAAGGAGAGAGACGGGTGCGAGAGCGTCAGTATTAAGCG
GGGGTGAATAAGATCGCGATGGGAAAAAATTCGGTTAAGGCCAGGGGGAAAGAAAAAATATAAATTAAAACATATAGTAT
GGGCAAGCAGGGAGCTAGAACGATTCGCAGTTAATCCTGGCCTGTTAGAAACATCAGAAGGCTGTAGACAAATACTGGGA
CAGCTACAACCATCCCTTCAGACAGGATCAGAAGAACTTAGATCATTATATAATACAGTAGCAACCCTCTATTGTGTGCA
TCAAAGGATAGAGATAAAAGACACCAAGGAAGCTTTAGACAAGATAGAGGAAGAGCAAAACAAAAGTAAGACCACCGCAC
AGCAAGCGGCCGCTGATCTTCAGACCTGGAGGAGGAGATATGAGGGACAATTGGAGAAGTGAATTATATAAATATAAAGT
AGTAAAAATTGAACCATTAGGAGTAGCACCCACCAAGGCAAAGAGAAGAGTGGTGCAGAGAGAAAAAAGAGCAGTGGGAA
TAGGAGCTTTGTTCCTTGGGTTCTTGGGAGCAGCAGGAAGCACTATGGGCGCAGCGTCAATGACGCTGACGGTACAGGCC
AGACAATTATTGTCTGGTATAGTGCAGCAGCAGAACAATTTGCTGAGGGCTATTGAGGCGCAACAGCATCTGTTGCAACT
CACAGTCTGGGGCATCAAGCAGCTCCAGGCAAGAATCCTGGCTGTGGAAAGATACCTAAAGGATCAACAGCTCCTGGGGA
TTTGGGGTTGCTCTGGAAAACTCATTTGCACCACTGCTGTGCCTTGGAATGCTAGTTGGAGTAATAAATCTCTGGAACAG
ATTTGGAATCACACGACCTGGATGGAGTGGGACAGAGAAATTAACAATTACACAAGCTTAATACACTCCTTAATTGAAGA
ATCGCAAAACCAGCAAGAAAAGAATGAACAAGAATTATTGGAATTAGATAAATGGGCAAGTTTGTGGAATTGGTTTAACA
TAACAAATTGGCTGTGGTATATAAAATTATTCATAATGATAGTAGGAGGCTTGGTAGGTTTAAGAATAGTTTTTGCTGTA
CTTTCTATAGTGAATAGAGTTAGGCAGGGATATTCACCATTATCGTTTCAGACCCACCTCCCAACCCCGAGGGGACCCGA
CAGGCCCGAAGGAATAGAAGAAGAAGGTGGAGAGAGAGACAGAGACAGATCCATTCGATTAGTGAACGGATCTCGACGGT
ATCGTTTTAAAAGAAAAGGGGGGATTGGGGGGTACAGTGCAGGGGAAAGAATAGTAGACATAATAGCAACAGACATACAA
ACTAAAGAATTACAAAAACAAATTACAAAAATTCAAAATTTTATCGGCGTGTTGGGGGTGGACCATCCTCTAGGTATTGA
ATAAGAAAAATGAAGTTAAGGTGGTTGATGGTAACACTATGCTAATAACTGCAGAGCCAGAAGCACCATAAGGGACATGA
TAAGGGAGCCAGCAGACCTCTGATCTCTTCCTGAATGCTAATCTTAAACATCCTGAGGAAGAATGGGACTTCCATTTGGG
GTGGGCCTATGATAGGGTAATAAGACAGTAGTGAATATCAAGCTACAAAAAGCCCCCTTTCAAATTCTTCTCAGTCCTAA
CTTTTCATACTAAGCCCAGTCCTTCCAAAGCAGACTGTGAAAGAGTGATAGTTCCGGGAGACTAGCACTGCAGATTCCGG
GTCACTGTGAGTGGGGGAGGCAGGGAAGAAGGGCTCACAGGACAGTCAAACCATGCCCCCTGTTTTTCCTTCTTCAAGTA
GACCTCTATAAGACAACAGAGACAACTAAGGCTGAGTGGCCAGGCGAGGAGAAACCATCTCGCCGTAAAACATGGAAGGA
ACACTTCAGGGGAAAGGTGGTATCTCTAAGCAAGAGAACTGAGTGGAGTCAAGGCTGAGAGATGCAGGATAAGCAAATGG
GTAGTGAAAAGACATTCATGAGGACAGCTAAAACAATAAGTAATGTAAAATACAGCATAGCAAAACTTTAACCTCCAAAT
CAAGCCTCTACTTGAATCCTTTTCTGAGGGATGAATAAGGCATAGGCATCAGGGGCTGTTGCCAATGTGCATTAGCTGTT
TGCAGCCTCACCTTCTTTCATGGAGTTTAAGATATAGTGTATTTTCCCAAGGTTTGAACTAGCTCTTCATTTCTTTATGT
TTTAAATGCACTGACCTCCCACATTCCCTTTTTAGTAAAATATTCAGAAATAATTTAAATACATCATTGCAATGAAAATA
AATGTTTTTTATTAGGCAGAATCCAGATGCTCAAGGCCCTTCATAATATCCCCCAGTTTAGTAGTTGGACTTAGGGAACA
AAGGAACCTTTAATAGAAATTGGACAGCAAGAAAGCGAGCTTAGTGATACTTGTGGGCCAGGGCATTAGCCACACCAGCC
ACCACTTTCTGATAGGCAGCCTGCACTGGTGGGGTGAATTCTTTGCCAAAGTGATGGGCCAGCACACAGACCAGCACGTT
GCCCAGGAGCTGTGGGAGGAAGATAAGAGGTATGAACATGATTAGCAAAAGGGCCTAGCTTGGACTCAGAATAATCCAGC
CTTATCCCAACCATAAAATAAAAGCAGAATGGTAGCTGGATTGTAGCTGCTATTAGCAATATGAAACCTCTTACATCAGT
TACAATTTATATGCAGAAATATTTATATGCAGAAATATTGCTATTGCCTTAACCCAGAAATTATCACTGTTATTCTTTAG
AATGGTGCAAAGAGGCATGATACATTGTATCATTATTGCCCTGAAAGAAAGAGATTAGGGAAAGTATTAGAAATAAGATA
AACAAAAAAGTATATTAAAAGAAGAAAGCATTTTTTAAAATTACAAATGCAAAATTACCCTGATTTGGTCAATATGTGTA
CCCTGTTACTTCTCCCCTTCCTATGACATGAACTTAACCATAGAAAAGAAGGGGAAAGAAAACATCAAGGGTCCCATAGA
CTCACCCTGAAGTTCTCAGGATCCACGTGCAGCTTGTCACAGTGCAGCTCACTCAGCTGGGCAAAGGTGCCCTTGAGGTT
GTCCAGGTGAGCCAGGCCATCACTAAAGGCACCGAGCACTTTCTTGCCATGAGCCTTCACCTTAGGGTTGCCCATAACAG
CATCAGGAGTGGACAGATCCCCAAAGGACTCAAAGAACCTCTGGGTCCAAGGGTAGACCACCAGCAGCCTAAGGGTGGGA
AAATAGACCAATAGGCAGAGAGAGTCAGTGCCTATCAGAAACCCAAGAGTCTTCTCTGTCTCCACATGCCCAGTTTCTAT
TGGTCTCCTTAAACCTGTCTTGTAACCTTGATACCAACCTGCCCAGGGCCTCACCACCAACTTCATCCACGTTCACCTTG
CCCCACAGGGCAGTAACGGCAGACTTCTCCTCAGGAGTCAGGTGCACCATGGTGTCTGTTTGAGGTTGCTAGTGAACACA
GTTGTGTCAGAAGCAAATGTAAGCAATAGATGGCTCTGCCCTGACTTTTATGCCCAGCCCTGGCTCCTGCCCTCCCTGCT
CCTGGGAGTAGATTGGCCAACCCTAGGGTGTGGCTCCACAGGGTGAGGTCTAAGTGATGACAGCCGTACCTGTCCTTGGC
TCTTCTGGCACTGGCTTAGGAGTTGGACTTCAAACCCTCAGCCCTCCCTCTAAGATATATCTCTTGGCCCCATACCATCA
GTACAAATTGCTACTAAAAACATCCTCCTTTGCAAGTGTATTTACGGCATCTGTGAAGGAAAGAAACATCTCCTCTAAAC
CACTATGCTGCTAGAGCCTCTTTTCTGTACTCAAGCCTCATTCAGACACTAGTGTCACCAGTCTCCTCATATACCTATTG
TATTTTCTTCTTCTTGCTGGTTTAGTCATGTTTTCTGGGAGCTTAGGGGCTTATTTTATTTTGTTTTGTTTTCTAATCAA
CAGAGATGGGCAAACCCATTATTTTTTTCTTTAGACTTGGGATGGTGATAGCTGGGCAGCGTCAGAAACTGTGTGTGGAT
ATAGATAAGAGCTCGGACTATGCTGAGCTGTGATGAGGGAGGGACCTAGCCAAAGGCAGTGAGAGTCAGAATGCTCCTGC
TATTGCCTTCTCAGTCCCCACGCTTGGTTTCTACACAAGTAGATACATAGAAAAGGCTATAGGTTAGTGTTTGAGAGTCC
TGCATGAGTTAGTTGCTCAGAAATGCCCGATAAATATGTTATGTGTGTTTATGTATATATATGTTTTATATATATATATG
TGTGTGTGTGTGTGTGTGTGTGTTGTGTTTACAAATATGTGATTATCATCAAAACGTGAGGGCTAAAGTGACCAGATAAC
TTGCAGGTCTAGAATATGTCACATTCTGTCTCAGGCATCCATTTTCTTTATGATGCCGTTTGAGGTGGAGTTTTAGTCAG
GTGGTCAGCTTCTCCTTTTTTTTGCCATCTGCCCTGTAAGCATCCTGCTGGGGACCCAGATAGGAGTCATCACTCTAGGC
TGAGAACATCTGGGCACACACCCTAAGCCTCAGCATGACTCATCATGACTCAGCATTGCTGTGCTTGAGCCAGAAGGTTT
GCTTAGAAGGTTACACAGAACCAGAAGGCGGGGGTGGGGCACTGACCCCGACAGGGGCCTGGCCAGAACTGCTCATGCTT
GGACTATGGGAGGTCACTAATGGAGACACACAGAAATGTAACAGGAACTAAGGAAAAACTGAAGCTTATTTAATCAGAGA
TGAGATGCTGGAAGGGATAGAGGGAGCTGAGCTTGTAAAAAGTATAGTAATCATTCAGCAAATGGTTTTGAAGCACCTGC
TGGATGCTAAACACTATTTTCAGTGCTTGAATCATAAATAAGAACAAAACATGTATCTTATTCCCCACAAGAGTCCAAGT
AAAAAATAACAGTTAATTATAATGTGCTCTGTCCCCCAGGCTGGAGTGCAGTGGCACGATCTCAGCTCACTGCAACCTCC
GCCTCCCGGGTTCAAGCAATTCTCCTGCCTCAGCCACCCTAATAGCTGGGATTACAGGTGCACACCACCATGCCAGGCTA
ATTTTTGTACTTTTTGTAGAGGCAGGGTATCACCATGTTGTCCAAGATGGTCTTGAACTCCTGAGCTCCAAGCAGTCCAC
CCACCTCAGCCTCCCAAAGTGCTATCTGCGGCCGCCTATCTGTACCACTAGTCTCGAGAAGCTTTCATCAAAAAAAGTCT
AACCAGCTGCATTCGACTTTGACTGCAGCAGCTGGTTAGAAGGTTCTACTGGAGGAGGGTCCCAGCCCATTGCTAAATTA
ACATCAGGCTCTGAGACTGGCAGTATATCTCTAACAGTGGTTGATGCTATCTTCTGGAACTTGCCTGCTACATTGAGACC
ACTGACCCATACATAGGAAGCCCATAGCTCTGTCCTGAACTGTTAGGCCACTGGTCCAGAGAGTGTGCATCTCCTTTGAT
CCTCATAATAACCCTATGAGATAGACACAATTATTACTCTTACTTTATAGATGATGATCCTGAAAACATAGGAGTCAAGG
CACTTGCCCCTAGCTGGGGGTATAGGGGAGCAGTCCCATGTAGTAGTAGAATGAAAAATGCTGCTATGCTGTGCCTCCCC
CACCTTTCCCATGTCTGCCCTCTACTCATGGTCTATCTCTCCTGGCTCCTGGGAGTCATGGACTCCACCCAGCACCACCA
ACCTGACCTAACCACCTATCTGAGCCTGCCAGCCTATAACCCATCTGGGCCCTGATAGCTGGTGGCCAGCCCTGACCCCA
CCCCACCCTCCCTGGAACCTCTGATAGACACATCTGGCACACCAGCTCGCAAAGTCACCGTGAGGGTCTTGTGTTTGCTG
AGTCAAAATTCCTTGAAATCCAAGTCCTTAGAGACTCCTGCTCCCAAATTTACAGTCATAGACTTCTTCATGGCTGTCTC
CTTTATCCACAGAATGATTCCTTTGCTTCATTGCCCCATCCATCTGATCCTCCTCATCAGTGCAGCACAGGGCCCATGAG
CAGTAGCTGCAGAGTCTCACATAGGTCTGGCACTGCCTCTGACATGTCCGACCTTAGGCAAATGCTTGACTCTTCTGAGC
TCAGTCTTGTCATGGCAAAACAAAGATAATAATAGTGTTTTTTTATGGAGTTAGCGTGAGGATGGAAAACAATAGCAAAA
TTGATTAGACTATAAAAGGTCTCAACAAATAGTAGTAGATTTTATCATCCATTAATCCTTCCCTCTCCTCTCTTACTCAT
CCCATCACGTATGCCTCTTAATTTTCCCTTACCTATAATAAGAGTTATTCCTCTTATTATATTCTTCTTATAGTGATTCT
GGATATCAAAGTGGGAATGAGGGGCAGGCCACTAACGAAGAAGATGTTTCTCAAAGAAGCCATTCTCCCCACATAGATCA
TCTCAGCAGGGTTCAGGAAGATAAAGGAGGATCAAGGTCGAAGGTAGGAACTAAGGAAGAACACTGGGCAAGTGGATCCT
AAATATATCATTTAAATGCATAAATAAGCAAACCCTGCTCGGGAATGGGAGGGAGAGTCTCTGGAGTCCACCCCTTCTCG
GCCCTGGCTCTGCAGATAGTGCTATCAAAGCCCTGACAGAGCCCTGCCCATTGCTGGGCCTTGGAGTGAGTCAGCCTAGT
AGAGAGGCAGGGCAAGCCATCTCATAGCTGCTGAGTGGGAGAGAGAAAAGGGCTCATTGTCTATAAACTCAGGTCATGGC
TATTCTTATTCTCACACTAAGAAAAAGAATGAGATGTCTACATATACCCTGCGTCCCCTCTTGTGTACTGGGGCCCCCAA
GAGCTCTCTAAAAGTGATGGCAAAGTCATTGCGCTAGATGCCATCCCATCTATTATAAACCTGCATTTGTCTCCACACAC
CAGTCATGGACAATAACCCTCCTCCCAGGTCCACGTGCTTGTCTTTGTATAATACTCAAGTAATTTCGGAAAATGTATTC
TTTCAATCTTGTTCTGTTATTCCTGTTTCAATGGCTTAGTAGAAAAAGTACATACTTGTTTTCCCATAAATTGACAATAG
ACAATTTCACATCAATGTCTATATGGGTCGTTGTGTTTGCTGTGTTTGCAAAAACTCACAATAACTTTATATTGTTACTA
CTCTAAGAAAGTTACAACATGGTGAATACAAGAGAAAGCTATTACAAGTCCAGAAAACAAAAGTTATCATCTTGAGGCCT
CAGCTTTCTAGGAATAATATCAATATTACAAAACGCGTCGACGGTACCGTTAACGATCTTAGCCACTTTTTAAAAGAAAA
GGGGGGACTGGAAGGGCTAATTCACTCCCAACGAAGACAAGATATCCTGCTAGTCCTTCCTTTCTAAATGACGAGAGAGA
CAGAAGAATTCTTCAAGGTTAGTGTGTCCAGCATGCAACCTTTCCTTCCTGGATGAGCATCCCTGGAGTAGGAGAGCCAG
CCTGCCTCCTGCGCTGGCACAGAGCCCGGTTCCCTAGACAACTGCCTCTCCAAATCTGATGTCCAGCGCCACCTGGTGTC
CACATCAAGCAGACACAATTAATAGTCAACCTGTTCAGGAAAACTGTGAGGGGGAAAAAAAAGAAAGAGGATTTATGAAG
GGAAAAGAAAGTTTAGAGGATATGCCACGATTGGCTAGCAGCTGCTTTTTGCCTGTACTGGGTCTCTCTGGTTAGACCAG
ATCTGAGCCTGGGAGCTCTCTGGCTAACTAGGGAACCCACTGCTTAAGCCTCAATAAAGCTTGCCTTGAGTGCTTCATCC
GGAATCAACCTCTGGATTACAAAATTTGTGAAAGATTGACTGGTATTCTTAACTATGTTGCTCCTTTTACGCTATGTGGA
TACGCTGCTTTAATGCCTTTGTATCATGCTATTGCTTCCCGTATGGCTTTCATTTTCTCCTCCTTGTATAAATCCTGGTT
GCTGTCTCTTTATGAGGAGTTGTGGCCCGTTGTCAGGCAACGTGGCGTGGTGTGCACTGTGTTTGCTGACGCAACCCCCA
CTGGTTGGGGCATTGCCACCACCTGTCAGCTCCTTTCCGGGACTTTCGCTTTCCCCCTCCCTATTGCCACGGCGGAACTC
ATCGCCGCCTGCCTTGCCCGCTGCTGGACAGGGGCTCGGCTGTTGGGCACTGACAATTCCGTGGTGTTGTCGGGGAAGCT
GACGTCCTTTCCATGGCTGCTCGCCTGTGTTGCCACCTGGATTCTGCGCGGGACGTCCTTCTGCTACGTCCCTTCGGCCC
TCAATCCAGCGGACCTTCCTTCCCGCGGCCTGCTGCCGGCTCTGCGGCCTCTTCCGCGTCTTCGCCTTCGCCCTCAGACG
AGTCGGATCTCCCTTTGGGCCGCCTCCCCGCCTGTCCGGTAGCTTGCCAGCCTCGACTGTGCCTTCTAGTTGCCAGCCGT
CTGTTGTTTGCCCCTCCCCCGTGCCTTCCTTGACCCTGGAAGGTGCCACTCCCACTGTCCTTTCCTAATAAAATGAGGAA
ATTGCATCGCATTGTCTGAGTAGGTGTCATTCTATTCTGGGGGGTGGGGTGGGGCAGGACAGCAAGGGGGAGGATTGGGA
AGACAATAGCAGGCATGCAAGCTTGGCGTAATCATGGTCATAGCTGTTTCCTGTGTGAAATTGTTATCCGCTCACAATTC
CACACAACATACGAGCCGGAAGCATAAAGTGTAAAGCCTGGGGTGCCTAATGAGTGAGCTAACTCACATTAATTGCGTTG
CGCTCACTGCCCGCTTTCCAGTCGGGAAACCTGTCGTGCCAGCTGCATTAATGAATCGGCCAACGCGCGGGGAGAGGCGG
TTTGCGTATTGGGCGCTCTTCCGCTTCCTCGCTCACTGACTCGCTGCGCTCGGTCGTTCGGCTGCGGCGAGCGGTATCAG
CTCACTCAAAGGCGGTAATACGGTTATCCACAGAATCAGGGGATAACGCAGGAAAGAACATGTGAGCAAAAGGCCAGCAA
AAGGCCAGGAACCGTAAAAAGGCCGCGTTGCTGGCGTTTTTCCATAGGCTCCGCCCCCCTGACGAGCATCACAAAAATCG
ACGCTCAAGTCAGAGGTGGCGAAACCCGACAGGACTATAAAGATACCAGGCGTTTCCCCCTGGAAGCTCCCTCGTGCGCT
CTCCTGTTCCGACCCTGCCGCTTACCGGATACCTGTCCGCCTTTCTCCCTTCGGGAAGCGTGGCGCTTTCTCATAGCTCA
CGCTGTAGGTATCTCAGTTCGGTGTAGGTCGTTCGCTCCAAGCTGGGCTGTGTGCACGAACCCCCCGTTCAGCCCGACCG
CTGCGCCTTATCCGGTAACTATCGTCTTGAGTCCAACCCGGTAAGACACGACTTATCGCCACTGGCAGCAGCCACTGGTA
ACAGGATTAGCAGAGCGAGGTATGTAGGCGGTGCTACAGAGTTCTTGAAGTGGTGGCCTAACTACGGCTACACTAGAAGA
ACAGTATTTGGTATCTGCGCTCTGCTGAAGCCAGTTACCTTCGGAAAAAGAGTTGGTAGCTCTTGATCCGGCAAACAAAC
CACCGCTGGTAGCGGTGGTTTTTTTGTTTGCAAGCAGCAGATTACGCGCAGAAAAAAAGGATCTCAAGAAGATCCTTTGA
TCTTTTCTACGGGGTCTGACGCTCAGTGGAACGAAAACTCACGTTAAGGGATTTTGGTCATGAGATTATCAAAAAGGATC
TTCACCTAGATCCTTTTAAATTAAAAATGAAGTTTTAAATCAATCTAAAGTATATATGAGTAAACTTGGTCTGACAGTTA
CCAATGCTTAATCAGTGAGGCACCTATCTCAGCGATCTGTCTATTTCGTTCATCCATAGTTGCCTGACTCCCCGTCGTGT
AGATAACTACGATACGGGAGGGCTTACCATCTGGCCCCAGTGCTGCAATGATACCGCGAGACCCACGCTCACCGGCTCCA
GATTTATCAGCAATAAACCAGCCAGCCGGAAGGGCCGAGCGCAGAAGTGGTCCTGCAACTTTATCCGCCTCCATCCAGTC
TATTAATTGTTGCCGGGAAGCTAGAGTAAGTAGTTCGCCAGTTAATAGTTTGCGCAACGTTGTTGCCATTGCTACAGGCA
TCGTGGTGTCACGCTCGTCGTTTGGTATGGCTTCATTCAGCTCCGGTTCCCAACGATCAAGGCGAGTTACATGATCCCCC
ATGTTGTGCAAAAAAGCGGTTAGCTCCTTCGGTCCTCCGATCGTTGTCAGAAGTAAGTTGGCCGCAGTGTTATCACTCAT
GGTTATGGCAGCACTGCATAATTCTCTTACTGTCATGCCATCCGTAAGATGCTTTTCTGTGACTGGTGAGTACTCAACCA
AGTCATTCTGAGAATAGTGTATGCGGCGACCGAGTTGCTCTTGCCCGGCGTCAATACGGGATAATACCGCGCCACATAGC
AGAACTTTAAAAGTGCTCATCATTGGAAAACGTTCTTCGGGGCAAAACTCTCAAGGATCTTACCGCTGTTGAGATCCAGT
TCGATGTAACCCACTCGTGCACCCAACTGATCTTCAGCATCTTTTACTTTCACCAGCGTTTCTGGGTGAGCAAAAACAGG
AAGGCAAAATGCCGCAAAAAAGGGAATAAGGGCGACACGGAAATGTTGAATACTCATACTCTTCCTTTTTCAATATTATT
GAAGCATTTATCAGGGTTATTGTCTCATGAGCGGATACATATTTGAATGTATTTAGAAAAATAAACAAATAGGGGTTCCG
CGCACATTTCCCCGAAAAGTGCCACCTGACGTCTAAGAAACCATTATTATCATGACATTAACCTATAAAAATAGGCGTAT
CACGAGGCCCTTTCGTCTCGCGCGTTTCGGTGATGACGGTGAAAACCTCTGACACATGCAGCTCCCGGAGACGGTCACAG
CTTGTCTGTAAGCGGATGCCGGGAGCAGACAAGCCCGTCAGGGCGCGTCAGCGGGTGTTGGCGGGTGTCGGGGCTGGCTT
AACTATGCGGCATCAGAGCAGATTGTACTGAGAGTGCACC
[SEQ ID NO: 39]
ATTGGCCATTGCATACGTTGTATCCATATCATAATATGTACATTTATATTGGCTCATGTCCAACATTACCGCCATGTTGA
CATTGATTATTGACTAGTTATTAATAGTAATCAATTACGGGGTCATTAGTTCATAGCCCATATATGGAGTTCCGCGTTAC
ATAACTTACGGTAAATGGCCCGCCTGGCTGACCGCCCAACGACCCCCGCCCATTGACGTCAATAATGACGTATGTTCCCA
TAGTAACGCCAATAGGGACTTTCCATTGACGTCAATGGGTGGAGTATTTACGGTAAACTGCCCACTTGGCAGTACATCAA
GTGTATCATATGCCAAGTACGCCCCCTATTGACGTCAATGACGGTAAATGGCCCGCCTGGCATTATGCCCAGTACATGAC
CTTATGGGACTTTCCTACTTGGCAGTACATCTACGTATTAGTCATCGCTATTACCATGGTGATGCGGTTTTGGCAGTACA
TCAATGGGCGTGGATAGCGGTTTGACTCACGGGGATTTCCAAGTCTCCACCCCATTGACGTCAATGGGAGTTTGTTTTGG
CACCAAAATCAACGGGACTTTCCAAAATGTCGTAACAACTCCGCCCCATTGACGCAAATGGGCGGTAGGCGTGTACGGTG
GGAGGTCTATATAAGCAGAGCTCGTTTAGTGAACCGGGGTCTCTCTGGTTAGACCAGATCTGAGCCTGGGAGCTCTCTGG
CTAACTAGGGAACCCACTGCTTAAGCCTCAATAAAGCTTGCCTTGAGTGCTTCAAGTAGTGTGTGCCCGTCTGTTGTGTG
ACTCTGGTAACTAGAGATCCCTCAGACCCTTTTAGTCAGTGTGGAAAATCTCTAGCAGTGGCGCCCGAACAGGGACTTGA
AAGCGAAAGGGAAACCAGAGGAGCTCTCTCGACGCAGGACTCGGCTTGCTGAAGCGCGCACGGCAAGAGGCGAGGGGCGG
CGACTGGTGAGTACGCCAAAAATTTTGACTAGCGGAGGCTAGAAGGAGAGAGACGGGTGCGAGAGCGTCAGTATTAAGCG
GGGGTGAATAAGATCGCGATGGGAAAAAATTCGGTTAAGGCCAGGGGGAAAGAAAAAATATAAATTAAAACATATAGTAT
GGGCAAGCAGGGAGCTAGAACGATTCGCAGTTAATCCTGGCCTGTTAGAAACATCAGAAGGCTGTAGACAAATACTGGGA
CAGCTACAACCATCCCTTCAGACAGGATCAGAAGAACTTAGATCATTATATAATACAGTAGCAACCCTCTATTGTGTGCA
TCAAAGGATAGAGATAAAAGACACCAAGGAAGCTTTAGACAAGATAGAGGAAGAGCAAAACAAAAGTAAGACCACCGCAC
AGCAAGCGGCCGCTGATCTTCAGACCTGGAGGAGGAGATATGAGGGACAATTGGAGAAGTGAATTATATAAATATAAAGT
AGTAAAAATTGAACCATTAGGAGTAGCACCCACCAAGGCAAAGAGAAGAGTGGTGCAGAGAGAAAAAAGAGCAGTGGGAA
TAGGAGCTTTGTTCCTTGGGTTCTTGGGAGCAGCAGGAAGCACTATGGGCGCAGCGTCAATGACGCTGACGGTACAGGCC
AGACAATTATTGTCTGGTATAGTGCAGCAGCAGAACAATTTGCTGAGGGCTATTGAGGCGCAACAGCATCTGTTGCAACT
CACAGTCTGGGGCATCAAGCAGCTCCAGGCAAGAATCCTGGCTGTGGAAAGATACCTAAAGGATCAACAGCTCCTGGGGA
TTTGGGGTTGCTCTGGAAAACTCATTTGCACCACTGCTGTGCCTTGGAATGCTAGTTGGAGTAATAAATCTCTGGAACAG
ATTTGGAATCACACGACCTGGATGGAGTGGGACAGAGAAATTAACAATTACACAAGCTTAATACACTCCTTAATTGAAGA
ATCGCAAAACCAGCAAGAAAAGAATGAACAAGAATTATTGGAATTAGATAAATGGGCAAGTTTGTGGAATTGGTTTAACA
TAACAAATTGGCTGTGGTATATAAAATTATTCATAATGATAGTAGGAGGCTTGGTAGGTTTAAGAATAGTTTTTGCTGTA
CTTTCTATAGTGAATAGAGTTAGGCAGGGATATTCACCATTATCGTTTCAGACCCACCTCCCAACCCCGAGGGGACCCGA
CAGGCCCGAAGGAATAGAAGAAGAAGGTGGAGAGAGAGACAGAGACAGATCCATTCGATTAGTGAACGGATCTCGACGGT
ATCGTTTTAAAAGAAAAGGGGGGATTGGGGGGTACAGTGCAGGGGAAAGAATAGTAGACATAATAGCAACAGACATACAA
ACTAAAGAATTACAAAAACAAATTACAAAAATTCAAAATTTTATCGGCGTGTTGGGGGTGGACCATCCTCTAGGTATTGA
ATAAGAAAAATGAAGTTAAGGTGGTTGATGGTAACACTATGCTAATAACTGCAGAGCCAGAAGCACCATAAGGGACATGA
TAAGGGAGCCAGCAGACCTCTGATCTCTTCCTGAATGCTAATCTTAAACATCCTGAGGAAGAATGGGACTTCCATTTGGG
GTGGGCCTATGATAGGGTAATAAGACAGTAGTGAATATCAAGCTACAAAAAGCCCCCTTTCAAATTCTTCTCAGTCCTAA
CTTTTCATACTAAGCCCAGTCCTTCCAAAGCAGACTGTGAAAGAGTGATAGTTCCGGGAGACTAGCACTGCAGATTCCGG
GTCACTGTGAGTGGGGGAGGCAGGGAAGAAGGGCTCACAGGACAGTCAAACCATGCCCCCTGTTTTTCCTTCTTCAAGTA
GACCTCTATAAGACAACAGAGACAACTAAGGCTGAGTGGCCAGGCGAGGAGAAACCATCTCGCCGTAAAACATGGAAGGA
ACACTTCAGGGGAAAGGTGGTATCTCTAAGCAAGAGAACTGAGTGGAGTCAAGGCTGAGAGATGCAGGATAAGCAAATGG
GTAGTGAAAAGACATTCATGAGGACAGCTAAAACAATAAGTAATGTAAAATACAGCATAGCAAAACTTTAACCTCCAAAT
CAAGCCTCTACTTGAATCCTTTTCTGAGGGATGAATAAGGCATAGGCATCAGGGGCTGTTGCCAATGTGCATTAGCTGTT
TGCAGCCTCACCTTCTTTCATGGAGTTTAAGATATAGTGTATTTTCCCAAGGTTTGAACTAGCTCTTCATTTCTTTATGT
TTTAAATGCACTGACCTCCCACATTCCCTTTTTAGTAAAATATTCAGAAATAATTTAAATACATCATTGCAATGAAAATA
AATGTTTTTTATTAGGCAGAATCCAGATGCTCAAGGCCCTTCATAATATCCCCCAGTTTAGTAGTTGGACTTAGGGAACA
AAGGAACCTTTAATAGAAATTGGACAGCAAGAAAGCGAGCTTAGTGATACTTGTGGGCCAGGGCATTAGCCACACCAGCC
ACCACTTTCTGATAGGCAGCCTGCACTGGTGGGGTGAATTCTTTGCCAAAGTGATGGGCCAGCACACAGACCAGCACGTT
GCCCAGGAGCTGTGGGAGGAAGATAAGAGGTATGAACATGATTAGCAAAAGGGCCTAGCTTGGACTCAGAATAATCCAGC
CTTATCCCAACCATAAAATAAAAGCAGAATGGTAGCTGGATTGTAGCTGCTATTAGCAATATGAAACCTCTTACATCAGT
TACAATTTATATGCAGAAATATTTATATGCAGAAATATTGCTATTGCCTTAACCCAGAAATTATCACTGTTATTCTTTAG
AATGGTGCAAAGAGGCATGATACATTGTATCATTATTGCCCTGAAAGAAAGAGATTAGGGAAAGTATTAGAAATAAGATA
AACAAAAAAGTATATTAAAAGAAGAAAGCATTTTTTAAAATTACAAATGCAAAATTACCCTGATTTGGTCAATATGTGTA
CCCTGTTACTTCTCCCCTTCCTATGACATGAACTTAACCATAGAAAAGAAGGGGAAAGAAAACATCAAGGGTCCCATAGA
CTCACCCTGAAGTTCTCAGGATCCACGTGCAGCTTGTCACAGTGCAGCTCACTCAGCTGGGCAAAGGTGCCCTTGAGGTT
GTCCAGGTGAGCCAGGCCATCACTAAAGGCACCGAGCACTTTCTTGCCATGAGCCTTCACCTTAGGGTTGCCCATAACAG
CATCAGGAGTGGACAGATCCCCAAAGGACTCAAAGAACCTCTGGGTCCAAGGGTAGACCACCAGCAGCCTAAGGGTGGGA
AAATAGACCAATAGGCAGAGAGAGTCAGTGCCTATCAGAAACCCAAGAGTCTTCTCTGTCTCCACATGCCCAGTTTCTAT
TGGTCTCCTTAAACCTGTCTTGTAACCTTGATACCAACCTGCCCAGGGCCTCACCACCAACTTCATCCACGTTCACCTTG
CCCCACAGGGCAGTAACGGCAGACTTCTCCTCAGGAGTCAGGTGCACCATGGTGTCTGTTTGAGGTTGCTAGTGAACACA
GTTGTGTCAGAAGCAAATGTAAGCAATAGATGGCTCTGCCCTGACTTTTATGCCCAGCCCTGGCTCCTGCCCTCCCTGCT
CCTGGGAGTAGATTGGCCAACCCTAGGGTGTGGCTCCACAGGGTGAGGTCTAAGTGATGACAGCCGTACCTGTCCTTGGC
TCTTCTGGCACTGGCTTAGGAGTTGGACTTCAAACCCTCAGCCCTCCCTCTAAGATATATCTCTTGGCCCCATACCATCA
GTACAAATTGCTACTAAAAACATCCTCCTTTGCAAGTGTATTTACTCTAGAATATGTCACATTCTGTCTCAGGCATCCAT
TTTCTTTATGATGCCGTTTGAGGTGGAGTTTTAGTCAGGTGGTCAGCTTCTCCTTTTTTTTGCCATCTGCCCTGTAAGCA
TCCTGCTGGGGACCCAGATAGGAGTCATCACTCTAGGCTGAGAACATCTGGGCACACACCCTAAGCCTCAGCATGACTCA
TCATGACTCAGCATTGCTGTGCTTGAGCCAGAAGGTTTGCTTAGAAGGTTACACAGAACCAGAAGGCGGGGGTGGGGCAC
TGACCCCGACAGGGGCCTGGCCAGAACTGCTCATGCTTGGACTATGGGAGGTCACTAATGGAGACACACAGAAATGTAAC
AGGAACTAAGGAAAAACTGAAGCTTATTTAATCAGAGATGAGATGCTGGAAGGGATAGAGGGAGCTGAGCTTGTAAAAAG
TATAGTAATCATTCAGCAAATGGTTTTGAAGCACCTGCTGGATGCTAAACACTATTTTCAGTGCTTGAATCATAAATAAG
AACAAAACATGTATCTTATTCCCCACAAGAGTCCAAGTAAAAAATAACAGTTAATTATAATGTGCTCTGTCCCCCAGGCT
GGAGTGCAGTGGCACGATCTCAGCTCACTGCAACCTCCGCCTCCCGGGTTCAAGCAATTCTCCTGCCTCAGCCACCCTAA
TAGCTGGGATTACAGGTGCACACCACCATGCCAGGCTAATTTTTGTACTTTTTGTAGAGGCAGGGTATCACCATGTTGTC
CAAGATGGTCTTGAACTCCTGAGCTCCAAGCAGTCCACCCACCTCAGCCTCCCAAAGTGCTATCTGCGGCCGCCTATCTG
TACCACTAGTCTCGAGAAGCTTTCATCAAAAAAAGTCTAACCAGCTGCATTCGACTTTGACTGCAGCAGCTGGTTAGAAG
GTTCTACTGGAGGAGGGTCCCAGCCCATTGCTAAATTAACATCAGGCTCTGAGACTGGCAGTATATCTCTAACAGTGGTT
GATGCTATCTTCTGGAACTTGCCTGCTACATTGAGACCACTGACCCATACATAGGAAGCCCATAGCTCTGTCCTGAACTG
TTAGGCCACTGGTCCAGAGAGTGTGCATCTCCTTTGATCCTCATAATAACCCTATGAGATAGACACAATTATTACTCTTA
CTTTATAGATGATGATCCTGAAAACATAGGAGTCAAGGCACTTGCCCCTAGCTGGGGGTATAGGGGAGCAGTCCCATGTA
GTAGTAGAATGAAAAATGCTGCTATGCTGTGCCTCCCCCACCTTTCCCATGTCTGCCCTCTACTCATGGTCTATCTCTCC
TGGCTCCTGGGAGTCATGGACTCCACCCAGCACCACCAACCTGACCTAACCACCTATCTGAGCCTGCCAGCCTATAACCC
ATCTGGGCCCTGATAGCTGGTGGCCAGCCCTGACCCCACCCCACCCTCCCTGGAACCTCTGATAGACACATCTGGCACAC
CAGCTCGCAAAGTCACCGTGAGGGTCTTGTGTTTGCTGAGTCAAAATTCCTTGAAATCCAAGTCCTTAGAGACTCCTGCT
CCCAAATTTACAGTCATAGACTTCTTCATGGCTGTCTCCTTTATCCACAGAATGATTCCTTTGCTTCATTGCCCCATCCA
TCTGATCCTCCTCATCAGTGCAGCACAGGGCCCATGAGCAGTAGCTGCAGAGTCTCACATAGGTCTGGCACTGCCTCTGA
CATGTCCGACCTTAGGCAAATGCTTGACTCTTCTGAGCTCAGTCTTGTCATGGCAAAACAAAGATAATAATAGTGTTTTT
TTATGGAGTTAGCGTGAGGATGGAAAACAATAGCAAAATTGATTAGACTATAAAAGGTCTCAACAAATAGTAGTAGATTT
TATCATCCATTAATCCTTCCCTCTCCTCTCTTACTCATCCCATCACGTATGCCTCTTAATTTTCCCTTACCTATAATAAG
AGTTATTCCTCTTATTATATTCTTCTTATAGTGATTCTGGATATCAAAGTGGGAATGAGGGGCAGGCCACTAACGAAGAA
GATGTTTCTCAAAGAAGCCATTCTCCCCACATAGATCATCTCAGCAGGGTTCAGGAAGATAAAGGAGGATCAAGGTCGAA
GGTAGGAACTAAGGAAGAACACTGGGCAAGTGGATCCTAAATATATCATTTAAATGCATAAATAAGCAAACCCTGCTCGG
GAATGGGAGGGAGAGTCTCTGGAGTCCACCCCTTCTCGGCCCTGGCTCTGCAGATAGTGCTATCAAAGCCCTGACAGAGC
CCTGCCCATTGCTGGGCCTTGGAGTGAGTCAGCCTAGTAGAGAGGCAGGGCAAGCCATCTCATAGCTGCTGAGTGGGAGA
GAGAAAAGGGCTCATTGTCTATAAACTCAGGTCATGGCTATTCTTATTCTCACACTAAGAAAAAGAATGAGATGTCTACA
TATACCCTGCGTCCCCTCTTGTGTACTGGGGCCCCCAAGAGCTCTCTAAAAGTGATGGCAAAGTCATTGCGCTAGATGCC
ATCCCATCTATTATAAACCTGCATTTGTCTCCACACACCAGTCATGGACAATAACCCTCCTCCCAGGTCCACGTGCTTGT
CTTTGTATAATACTCAAGTAATTTCGGAAAATGTATTCTTTCAATCTTGTTCTGTTATTCCTGTTTCAATGGCTTAGTAG
AAAAAGTACATACTTGTTTTCCCATAAATTGACAATAGACAATTTCACATCAATGTCTATATGGGTCGTTGTGTTTGCTG
TGTTTGCAAAAACTCACAATAACTTTATATTGTTACTACTCTAAGAAAGTTACAACATGGTGAATACAAGAGAAAGCTAT
TACAAGTCCAGAAAACAAAAGTTATCATCTTGAGGCCTCAGCTTTCTAGGAATAATATCAATATTACAAAACGCGTCGAC
GGTACCGTTAACGATCTTAGCCACTTTTTAAAAGAAAAGGGGGGACTGGAAGGGCTAATTCACTCCCAACGAAGACAAGA
TATCCTGCTAGTCCTTCCTTTCTAAATGACGAGAGAGACAGAAGAATTCTTCAAGGTTAGTGTGTCCAGCATGCAACCTT
TCCTTCCTGGATGAGCATCCCTGGAGTAGGAGAGCCAGCCTGCCTCCTGCGCTGGCACAGAGCCCGGTTCCCTAGACAAC
TGCCTCTCCAAATCTGATGTCCAGCGCCACCTGGTGTCCACATCAAGCAGACACAATTAATAGTCAACCTGTTCAGGAAA
ACTGTGAGGGGGAAAAAAAAGAAAGAGGATTTATGAAGGGAAAAGAAAGTTTAGAGGATATGCCACGATTGGCTAGCAGC
TGCTTTTTGCCTGTACTGGGTCTCTCTGGTTAGACCAGATCTGAGCCTGGGAGCTCTCTGGCTAACTAGGGAACCCACTG
CTTAAGCCTCAATAAAGCTTGCCTTGAGTGCTTCATCCGGAATCAACCTCTGGATTACAAAATTTGTGAAAGATTGACTG
GTATTCTTAACTATGTTGCTCCTTTTACGCTATGTGGATACGCTGCTTTAATGCCTTTGTATCATGCTATTGCTTCCCGT
ATGGCTTTCATTTTCTCCTCCTTGTATAAATCCTGGTTGCTGTCTCTTTATGAGGAGTTGTGGCCCGTTGTCAGGCAACG
TGGCGTGGTGTGCACTGTGTTTGCTGACGCAACCCCCACTGGTTGGGGCATTGCCACCACCTGTCAGCTCCTTTCCGGGA
CTTTCGCTTTCCCCCTCCCTATTGCCACGGCGGAACTCATCGCCGCCTGCCTTGCCCGCTGCTGGACAGGGGCTCGGCTG
TTGGGCACTGACAATTCCGTGGTGTTGTCGGGGAAGCTGACGTCCTTTCCATGGCTGCTCGCCTGTGTTGCCACCTGGAT
TCTGCGCGGGACGTCCTTCTGCTACGTCCCTTCGGCCCTCAATCCAGCGGACCTTCCTTCCCGCGGCCTGCTGCCGGCTC
TGCGGCCTCTTCCGCGTCTTCGCCTTCGCCCTCAGACGAGTCGGATCTCCCTTTGGGCCGCCTCCCCGCCTGTCCGGTAG
CTTGCCAGCCTCGACTGTGCCTTCTAGTTGCCAGCCGTCTGTTGTTTGCCCCTCCCCCGTGCCTTCCTTGACCCTGGAAG
GTGCCACTCCCACTGTCCTTTCCTAATAAAATGAGGAAATTGCATCGCATTGTCTGAGTAGGTGTCATTCTATTCTGGGG
GGTGGGGTGGGGCAGGACAGCAAGGGGGAGGATTGGGAAGACAATAGCAGGCATGCAAGCTTGGCGTAATCATGGTCATA
GCTGTTTCCTGTGTGAAATTGTTATCCGCTCACAATTCCACACAACATACGAGCCGGAAGCATAAAGTGTAAAGCCTGGG
GTGCCTAATGAGTGAGCTAACTCACATTAATTGCGTTGCGCTCACTGCCCGCTTTCCAGTCGGGAAACCTGTCGTGCCAG
CTGCATTAATGAATCGGCCAACGCGCGGGGAGAGGCGGTTTGCGTATTGGGCGCTCTTCCGCTTCCTCGCTCACTGACTC
GCTGCGCTCGGTCGTTCGGCTGCGGCGAGCGGTATCAGCTCACTCAAAGGCGGTAATACGGTTATCCACAGAATCAGGGG
ATAACGCAGGAAAGAACATGTGAGCAAAAGGCCAGCAAAAGGCCAGGAACCGTAAAAAGGCCGCGTTGCTGGCGTTTTTC
CATAGGCTCCGCCCCCCTGACGAGCATCACAAAAATCGACGCTCAAGTCAGAGGTGGCGAAACCCGACAGGACTATAAAG
ATACCAGGCGTTTCCCCCTGGAAGCTCCCTCGTGCGCTCTCCTGTTCCGACCCTGCCGCTTACCGGATACCTGTCCGCCT
TTCTCCCTTCGGGAAGCGTGGCGCTTTCTCATAGCTCACGCTGTAGGTATCTCAGTTCGGTGTAGGTCGTTCGCTCCAAG
CTGGGCTGTGTGCACGAACCCCCCGTTCAGCCCGACCGCTGCGCCTTATCCGGTAACTATCGTCTTGAGTCCAACCCGGT
AAGACACGACTTATCGCCACTGGCAGCAGCCACTGGTAACAGGATTAGCAGAGCGAGGTATGTAGGCGGTGCTACAGAGT
TCTTGAAGTGGTGGCCTAACTACGGCTACACTAGAAGAACAGTATTTGGTATCTGCGCTCTGCTGAAGCCAGTTACCTTC
GGAAAAAGAGTTGGTAGCTCTTGATCCGGCAAACAAACCACCGCTGGTAGCGGTGGTTTTTTTGTTTGCAAGCAGCAGAT
TACGCGCAGAAAAAAAGGATCTCAAGAAGATCCTTTGATCTTTTCTACGGGGTCTGACGCTCAGTGGAACGAAAACTCAC
GTTAAGGGATTTTGGTCATGAGATTATCAAAAAGGATCTTCACCTAGATCCTTTTAAATTAAAAATGAAGTTTTAAATCA
ATCTAAAGTATATATGAGTAAACTTGGTCTGACAGTTACCAATGCTTAATCAGTGAGGCACCTATCTCAGCGATCTGTCT
ATTTCGTTCATCCATAGTTGCCTGACTCCCCGTCGTGTAGATAACTACGATACGGGAGGGCTTACCATCTGGCCCCAGTG
CTGCAATGATACCGCGAGACCCACGCTCACCGGCTCCAGATTTATCAGCAATAAACCAGCCAGCCGGAAGGGCCGAGCGC
AGAAGTGGTCCTGCAACTTTATCCGCCTCCATCCAGTCTATTAATTGTTGCCGGGAAGCTAGAGTAAGTAGTTCGCCAGT
TAATAGTTTGCGCAACGTTGTTGCCATTGCTACAGGCATCGTGGTGTCACGCTCGTCGTTTGGTATGGCTTCATTCAGCT
CCGGTTCCCAACGATCAAGGCGAGTTACATGATCCCCCATGTTGTGCAAAAAAGCGGTTAGCTCCTTCGGTCCTCCGATC
GTTGTCAGAAGTAAGTTGGCCGCAGTGTTATCACTCATGGTTATGGCAGCACTGCATAATTCTCTTACTGTCATGCCATC
CGTAAGATGCTTTTCTGTGACTGGTGAGTACTCAACCAAGTCATTCTGAGAATAGTGTATGCGGCGACCGAGTTGCTCTT
GCCCGGCGTCAATACGGGATAATACCGCGCCACATAGCAGAACTTTAAAAGTGCTCATCATTGGAAAACGTTCTTCGGGG
CAAAACTCTCAAGGATCTTACCGCTGTTGAGATCCAGTTCGATGTAACCCACTCGTGCACCCAACTGATCTTCAGCATCT
TTTACTTTCACCAGCGTTTCTGGGTGAGCAAAAACAGGAAGGCAAAATGCCGCAAAAAAGGGAATAAGGGCGACACGGAA
ATGTTGAATACTCATACTCTTCCTTTTTCAATATTATTGAAGCATTTATCAGGGTTATTGTCTCATGAGCGGATACATAT
TTGAATGTATTTAGAAAAATAAACAAATAGGGGTTCCGCGCACATTTCCCCGAAAAGTGCCACCTGACGTCTAAGAAACC
ATTATTATCATGACATTAACCTATAAAAATAGGCGTATCACGAGGCCCTTTCGTCTCGCGCGTTTCGGTGATGACGGTGA
AAACCTCTGACACATGCAGCTCCCGGAGACGGTCACAGCTTGTCTGTAAGCGGATGCCGGGAGCAGACAAGCCCGTCAGG
GCGCGTCAGCGGGTGTTGGCGGGTGTCGGGGCTGGCTTAACTATGCGGCATCAGAGCAGATTGTACTGAGAGTGCACC
[SEQ ID NO: 40]
ATTGGCCATTGCATACGTTGTATCCATATCATAATATGTACATTTATATTGGCTCATGTCCAACATTACCGCCATGTTGA
CATTGATTATTGACTAGTTATTAATAGTAATCAATTACGGGGTCATTAGTTCATAGCCCATATATGGAGTTCCGCGTTAC
ATAACTTACGGTAAATGGCCCGCCTGGCTGACCGCCCAACGACCCCCGCCCATTGACGTCAATAATGACGTATGTTCCCA
TAGTAACGCCAATAGGGACTTTCCATTGACGTCAATGGGTGGAGTATTTACGGTAAACTGCCCACTTGGCAGTACATCAA
GTGTATCATATGCCAAGTACGCCCCCTATTGACGTCAATGACGGTAAATGGCCCGCCTGGCATTATGCCCAGTACATGAC
CTTATGGGACTTTCCTACTTGGCAGTACATCTACGTATTAGTCATCGCTATTACCATGGTGATGCGGTTTTGGCAGTACA
TCAATGGGCGTGGATAGCGGTTTGACTCACGGGGATTTCCAAGTCTCCACCCCATTGACGTCAATGGGAGTTTGTTTTGG
CACCAAAATCAACGGGACTTTCCAAAATGTCGTAACAACTCCGCCCCATTGACGCAAATGGGCGGTAGGCGTGTACGGTG
GGAGGTCTATATAAGCAGAGCTCGTTTAGTGAACCGGGGTCTCTCTGGTTAGACCAGATCTGAGCCTGGGAGCTCTCTGG
CTAACTAGGGAACCCACTGCTTAAGCCTCAATAAAGCTTGCCTTGAGTGCTTCAAGTAGTGTGTGCCCGTCTGTTGTGTG
ACTCTGGTAACTAGAGATCCCTCAGACCCTTTTAGTCAGTGTGGAAAATCTCTAGCAGTGGCGCCCGAACAGGGACTTGA
AAGCGAAAGGGAAACCAGAGGAGCTCTCTCGACGCAGGACTCGGCTTGCTGAAGCGCGCACGGCAAGAGGCGAGGGGCGG
CGACTGGTGAGTACGCCAAAAATTTTGACTAGCGGAGGCTAGAAGGAGAGAGACGGGTGCGAGAGCGTCAGTATTAAGCG
GGGGTGAATAAGATCGCGATGGGAAAAAATTCGGTTAAGGCCAGGGGGAAAGAAAAAATATAAATTAAAACATATAGTAT
GGGCAAGCAGGGAGCTAGAACGATTCGCAGTTAATCCTGGCCTGTTAGAAACATCAGAAGGCTGTAGACAAATACTGGGA
CAGCTACAACCATCCCTTCAGACAGGATCAGAAGAACTTAGATCATTATATAATACAGTAGCAACCCTCTATTGTGTGCA
TCAAAGGATAGAGATAAAAGACACCAAGGAAGCTTTAGACAAGATAGAGGAAGAGCAAAACAAAAGTAAGACCACCGCAC
AGCAAGCGGCCGCTGATCTTCAGACCTGGAGGAGGAGATATGAGGGACAATTGGAGAAGTGAATTATATAAATATAAAGT
AGTAAAAATTGAACCATTAGGAGTAGCACCCACCAAGGCAAAGAGAAGAGTGGTGCAGAGAGAAAAAAGAGCAGTGGGAA
TAGGAGCTTTGTTCCTTGGGTTCTTGGGAGCAGCAGGAAGCACTATGGGCGCAGCGTCAATGACGCTGACGGTACAGGCC
AGACAATTATTGTCTGGTATAGTGCAGCAGCAGAACAATTTGCTGAGGGCTATTGAGGCGCAACAGCATCTGTTGCAACT
CACAGTCTGGGGCATCAAGCAGCTCCAGGCAAGAATCCTGGCTGTGGAAAGATACCTAAAGGATCAACAGCTCCTGGGGA
TTTGGGGTTGCTCTGGAAAACTCATTTGCACCACTGCTGTGCCTTGGAATGCTAGTTGGAGTAATAAATCTCTGGAACAG
ATTTGGAATCACACGACCTGGATGGAGTGGGACAGAGAAATTAACAATTACACAAGCTTAATACACTCCTTAATTGAAGA
ATCGCAAAACCAGCAAGAAAAGAATGAACAAGAATTATTGGAATTAGATAAATGGGCAAGTTTGTGGAATTGGTTTAACA
TAACAAATTGGCTGTGGTATATAAAATTATTCATAATGATAGTAGGAGGCTTGGTAGGTTTAAGAATAGTTTTTGCTGTA
CTTTCTATAGTGAATAGAGTTAGGCAGGGATATTCACCATTATCGTTTCAGACCCACCTCCCAACCCCGAGGGGACCCGA
CAGGCCCGAAGGAATAGAAGAAGAAGGTGGAGAGAGAGACAGAGACAGATCCATTCGATTAGTGAACGGATCTCGACGGT
ATCGTTTTAAAAGAAAAGGGGGGATTGGGGGGTACAGTGCAGGGGAAAGAATAGTAGACATAATAGCAACAGACATACAA
ACTAAAGAATTACAAAAACAAATTACAAAAATTCAAAATTTTATCGGCGTGTTGGGGGTGGACCATCCTCTAGGTATTGA
ATAAGAAAAATGAAGTTAAGGTGGTTGATGGTAACACTATGCTAATAACTGCAGAGCCAGAAGCACCATAAGGGACATGA
TAAGGGAGCCAGCAGACCTCTGATCTCTTCCTGAATGCTAATCTTAAACATCCTGAGGAAGAATGGGACTTCCATTTGGG
GTGGGCCTATGATAGGGTAATAAGACAGTAGTGAATATCAAGCTACAAAAAGCCCCCTTTCAAATTCTTCTCAGTCCTAA
CTTTTCATACTAAGCCCAGTCCTTCCAAAGCAGACTGTGAAAGAGTGATAGTTCCGGGAGACTAGCACTGCAGATTCCGG
GTCACTGTGAGTGGGGGAGGCAGGGAAGAAGGGCTCACAGGACAGTCAAACCATGCCCCCTGTTTTTCCTTCTTCAAGTA
GACCTCTATAAGACAACAGAGACAACTAAGGCTGAGTGGCCAGGCGAGGAGAAACCATCTCGCCGTAAAACATGGAAGGA
ACACTTCAGGGGAAAGGTGGTATCTCTAAGCAAGAGAACTGAGTGGAGTCAAGGCTGAGAGATGCAGGATAAGCAAATGG
GTAGTGAAAAGACATTCATGAGGACAGCTAAAACAATAAGTAATGTAAAATACAGCATAGCAAAACTTTAACCTCCAAAT
CAAGCCTCTACTTGAATCCTTTTCTGAGGGATGAATAAGGCATAGGCATCAGGGGCTGTTGCCAATGTGCATTAGCTGTT
TGCAGCCTCACCTTCTTTCATGGAGTTTAAGATATAGTGTATTTTCCCAAGGTTTGAACTAGCTCTTCATTTCTTTATGT
TTTAAATGCACTGACCTCCCACATTCCCTTTTTAGTAAAATATTCAGAAATAATTTAAATACATCATTGCAATGAAAATA
AATGTTTTTTATTAGGCAGAATCCAGATGCTCAAGGCCCTTCATAATATCCCCCAGTTTAGTAGTTGGACTTAGGGAACA
AAGGAACCTTTAATAGAAATTGGACAGCAAGAAAGCGAGCTTAGTGATACTTGTGGGCCAGGGCATTAGCCACACCAGCC
ACCACTTTCTGATAGGCAGCCTGCACTGGTGGGGTGAATTCTTTGCCAAAGTGATGGGCCAGCACACAGACCAGCACGTT
GCCCAGGAGCTGTGGGAGGAAGATAAGAGGTATGAACATGATTAGCAAAAGGGCCTAGCTTGGACTCAGAATAATCCAGC
CTTATCCCAACCATAAAATAAAAGCAGAATGGTAGCTGGATTGTAGCTGCTATTAGCAATATGAAACCTCTTACATCAGT
TACAATTTATATGCAGAAATATTTATATGCAGAAATATTGCTATTGCCTTAACCCAGAAATTATCACTGTTATTCTTTAG
AATGGTGCAAAGAGGCATGATACATTGTATCATTATTGCCCTGAAAGAAAGAGATTAGGGAAAGTATTAGAAATAAGATA
AACAAAAAAGTATATTAAAAGAAGAAAGCATTTTTTAAAATTACAAATGCAAAATTACCCTGATTTGGTCAATATGTGTA
CCCTGTTACTTCTCCCCTTCCTATGACATGAACTTAACCATAGAAAAGAAGGGGAAAGAAAACATCAAGGGTCCCATAGA
CTCACCCTGAAGTTCTCAGGATCCACGTGCAGCTTGTCACAGTGCAGCTCACTCAGCTGGGCAAAGGTGCCCTTGAGGTT
GTCCAGGTGAGCCAGGCCATCACTAAAGGCACCGAGCACTTTCTTGCCATGAGCCTTCACCTTAGGGTTGCCCATAACAG
CATCAGGAGTGGACAGATCCCCAAAGGACTCAAAGAACCTCTGGGTCCAAGGGTAGACCACCAGCAGCCTAAGGGTGGGA
AAATAGACCAATAGGCAGAGAGAGTCAGTGCCTATCAGAAACCCAAGAGTCTTCTCTGTCTCCACATGCCCAGTTTCTAT
TGGTCTCCTTAAACCTGTCTTGTAACCTTGATACCAACCTGCCCAGGGCCTCACCACCAACTTCATCCACGTTCACCTTG
CCCCACAGGGCAGTAACGGCAGACTTCTCCTCAGGAGTCAGGTGCACCATGGTGTCTGTTTGAGGTTGCTAGTGAACACA
GTTGTGTCAGAAGCAAATGTAAGCAATAGATGGCTCTGCCCTGACTTTTATGCCCAGCCCTGGCTCCTGCCCTCCCTGCT
CCTGGGAGTAGATTGGCCAACCCTAGGGTGTGGCTCCACAGGGTGAGGTCTAAGTGATGACAGCCGTACCTGTCCTTGGC
TCTTCTGGCACTGGCTTAGGAGTTGGACTTCAAACCCTCAGCCCTCCCTCTAAGATATATCTCTTGGCCCCATACCATCA
GTACAAATTGCTACTAAAAACATCCTCCTTTGCAAGTGTATTTACGGCATCTGTGAAGGAAAGAAACATCTCCTCTAAAC
CACTATGCTGCTAGAGCCTCTTTTCTGTACTCAAGCCTCATTCAGACACTAGTGTCACCAGTCTCCTCATATACCTATTG
TATTTTCTTCTTCTTGCTGGTTTAGTCATGTTTTCTGGGAGCTTAGGGGCTTATTTTATTTTGTTTTGTTTTCTAATCAA
CAGAGATGGGCAAACCCATTATTTTTTTCTTTAGACTTGGGATGGTGATAGCTGGGCAGCGTCAGAAACTGTGTGTGGAT
ATAGATAAGAGCTCGGACTATGCTGAGCTGTGATGAGGGAGGGACCTAGCCAAAGGCAGTGAGAGTCAGAATGCTCCTGC
TATTGCCTTCTCAGTCCCCACGCTTGGTTTCTACACAAGTAGATACATAGAAAAGGCTATAGGTTAGTGTTTGAGAGTCC
TGCATGAGTTAGTTGCTCAGAAATGCCCGATAAATATGTTATGTGTGTTTATGTATATATATGTTTTATATATATATATG
TGTGTGTGTGTGTGTGTGTGTGTTGTGTTTACAAATATGTGATTATCATCAAAACGTGAGGGCTAAAGTGACCAGATAAC
TTGCAGGTCTAGACACCCTTTTCCGGCACGCAGATAGTCAATATCTTCAGCGTCCCCAAGGCCTGCAAGGGTGGGGCCCC
ATATCTGGAAGTCCCAGGCGGAGCTGGGAGTTGGTCAAGTCTGGGCTGTGGGGGCAGGGAGTGCTGGGGGATGGCTCGAG
AAGCTTTCATCAAAAAAAGTCTAACCAGCTGCATTCGACTTTGACTGCAGCAGCTGGTTAGAAGGTTCTACTGGAGGAGG
GTCCCAGCCCATTGCTAAATTAACATCAGGCTCTGAGACTGGCAGTATATCTCTAACAGTGGTTGATGCTATCTTCTGGA
ACTTGCCTGCTACATTGAGACCACTGACCCATACATAGGAAGCCCATAGCTCTGTCCTGAACTGTTAGGCCACTGGTCCA
GAGAGTGTGCATCTCCTTTGATCCTCATAATAACCCTATGAGATAGACACAATTATTACTCTTACTTTATAGATGATGAT
CCTGAAAACATAGGAGTCAAGGCACTTGCCCCTAGCTGGGGGTATAGGGGAGCAGTCCCATGTAGTAGTAGAATGAAAAA
TGCTGCTATGCTGTGCCTCCCCCACCTTTCCCATGTCTGCCCTCTACTCATGGTCTATCTCTCCTGGCTCCTGGGAGTCA
TGGACTCCACCCAGCACCACCAACCTGACCTAACCACCTATCTGAGCCTGCCAGCCTATAACCCATCTGGGCCCTGATAG
CTGGTGGCCAGCCCTGACCCCACCCCACCCTCCCTGGAACCTCTGATAGACACATCTGGCACACCAGCTCGCAAAGTCAC
CGTGAGGGTCTTGTGTTTGCTGAGTCAAAATTCCTTGAAATCCAAGTCCTTAGAGACTCCTGCTCCCAAATTTACAGTCA
TAGACTTCTTCATGGCTGTCTCCTTTATCCACAGAATGATTCCTTTGCTTCATTGCCCCATCCATCTGATCCTCCTCATC
AGTGCAGCACAGGGCCCATGAGCAGTAGCTGCAGAGTCTCACATAGGTCTGGCACTGCCTCTGACATGTCCGACCTTAGG
CAAATGCTTGACTCTTCTGAGCTCAGTCTTGTCATGGCAAAACAAAGATAATAATAGTGTTTTTTTATGGAGTTAGCGTG
AGGATGGAAAACAATAGCAAAATTGATTAGACTATAAAAGGTCTCAACAAATAGTAGTAGATTTTATCATCCATTAATCC
TTCCCTCTCCTCTCTTACTCATCCCATCACGTATGCCTCTTAATTTTCCCTTACCTATAATAAGAGTTATTCCTCTTATT
ATATTCTTCTTATAGTGATTCTGGATATCAAAGTGGGAATGAGGGGCAGGCCACTAACGAAGAAGATGTTTCTCAAAGAA
GCCATTCTCCCCACATAGATCATCTCAGCAGGGTTCAGGAAGATAAAGGAGGATCAAGGTCGAAGGTAGGAACTAAGGAA
GAACACTGGGCAAGTGGATCCTAAATATATCATTTAAATGCATAAATAAGCAAACCCTGCTCGGGAATGGGAGGGAGAGT
CTCTGGAGTCCACCCCTTCTCGGCCCTGGCTCTGCAGATAGTGCTATCAAAGCCCTGACAGAGCCCTGCCCATTGCTGGG
CCTTGGAGTGAGTCAGCCTAGTAGAGAGGCAGGGCAAGCCATCTCATAGCTGCTGAGTGGGAGAGAGAAAAGGGCTCATT
GTCTATAAACTCAGGTCATGGCTATTCTTATTCTCACACTAAGAAAAAGAATGAGATGTCTACATATACCCTGCGTCCCC
TCTTGTGTACTGGGGCCCCCAAGAGCTCTCTAAAAGTGATGGCAAAGTCATTGCGCTAGATGCCATCCCATCTATTATAA
ACCTGCATTTGTCTCCACACACCAGTCATGGACAATAACCCTCCTCCCAGGTCCACGTGCTTGTCTTTGTATAATACTCA
AGTAATTTCGGAAAATGTATTCTTTCAATCTTGTTCTGTTATTCCTGTTTCAATGGCTTAGTAGAAAAAGTACATACTTG
TTTTCCCATAAATTGACAATAGACAATTTCACATCAATGTCTATATGGGTCGTTGTGTTTGCTGTGTTTGCAAAAACTCA
CAATAACTTTATATTGTTACTACTCTAAGAAAGTTACAACATGGTGAATACAAGAGAAAGCTATTACAAGTCCAGAAAAC
AAAAGTTATCATCTTGAGGCCTCAGCTTTCTAGGAATAATATCAATATTACAAAACGCGTCGACGGTACCGTTAACGATC
TTAGCCACTTTTTAAAAGAAAAGGGGGGACTGGAAGGGCTAATTCACTCCCAACGAAGACAAGATATCCTGCTAGTCCTT
CCTTTCTAAATGACGAGAGAGACAGAAGAATTCTTCAAGGTTAGTGTGTCCAGCATGCAACCTTTCCTTCCTGGATGAGC
ATCCCTGGAGTAGGAGAGCCAGCCTGCCTCCTGCGCTGGCACAGAGCCCGGTTCCCTAGACAACTGCCTCTCCAAATCTG
ATGTCCAGCGCCACCTGGTGTCCACATCAAGCAGACACAATTAATAGTCAACCTGTTCAGGAAAACTGTGAGGGGGAAAA
AAAAGAAAGAGGATTTATGAAGGGAAAAGAAAGTTTAGAGGATATGCCACGATTGGCTAGCAGCTGCTTTTTGCCTGTAC
TGGGTCTCTCTGGTTAGACCAGATCTGAGCCTGGGAGCTCTCTGGCTAACTAGGGAACCCACTGCTTAAGCCTCAATAAA
GCTTGCCTTGAGTGCTTCATCCGGAATCAACCTCTGGATTACAAAATTTGTGAAAGATTGACTGGTATTCTTAACTATGT
TGCTCCTTTTACGCTATGTGGATACGCTGCTTTAATGCCTTTGTATCATGCTATTGCTTCCCGTATGGCTTTCATTTTCT
CCTCCTTGTATAAATCCTGGTTGCTGTCTCTTTATGAGGAGTTGTGGCCCGTTGTCAGGCAACGTGGCGTGGTGTGCACT
GTGTTTGCTGACGCAACCCCCACTGGTTGGGGCATTGCCACCACCTGTCAGCTCCTTTCCGGGACTTTCGCTTTCCCCCT
CCCTATTGCCACGGCGGAACTCATCGCCGCCTGCCTTGCCCGCTGCTGGACAGGGGCTCGGCTGTTGGGCACTGACAATT
CCGTGGTGTTGTCGGGGAAGCTGACGTCCTTTCCATGGCTGCTCGCCTGTGTTGCCACCTGGATTCTGCGCGGGACGTCC
TTCTGCTACGTCCCTTCGGCCCTCAATCCAGCGGACCTTCCTTCCCGCGGCCTGCTGCCGGCTCTGCGGCCTCTTCCGCG
TCTTCGCCTTCGCCCTCAGACGAGTCGGATCTCCCTTTGGGCCGCCTCCCCGCCTGTCCGGTAGCTTGCCAGCCTCGACT
GTGCCTTCTAGTTGCCAGCCGTCTGTTGTTTGCCCCTCCCCCGTGCCTTCCTTGACCCTGGAAGGTGCCACTCCCACTGT
CCTTTCCTAATAAAATGAGGAAATTGCATCGCATTGTCTGAGTAGGTGTCATTCTATTCTGGGGGGTGGGGTGGGGCAGG
ACAGCAAGGGGGAGGATTGGGAAGACAATAGCAGGCATGCAAGCTTGGCGTAATCATGGTCATAGCTGTTTCCTGTGTGA
AATTGTTATCCGCTCACAATTCCACACAACATACGAGCCGGAAGCATAAAGTGTAAAGCCTGGGGTGCCTAATGAGTGAG
CTAACTCACATTAATTGCGTTGCGCTCACTGCCCGCTTTCCAGTCGGGAAACCTGTCGTGCCAGCTGCATTAATGAATCG
GCCAACGCGCGGGGAGAGGCGGTTTGCGTATTGGGCGCTCTTCCGCTTCCTCGCTCACTGACTCGCTGCGCTCGGTCGTT
CGGCTGCGGCGAGCGGTATCAGCTCACTCAAAGGCGGTAATACGGTTATCCACAGAATCAGGGGATAACGCAGGAAAGAA
CATGTGAGCAAAAGGCCAGCAAAAGGCCAGGAACCGTAAAAAGGCCGCGTTGCTGGCGTTTTTCCATAGGCTCCGCCCCC
CTGACGAGCATCACAAAAATCGACGCTCAAGTCAGAGGTGGCGAAACCCGACAGGACTATAAAGATACCAGGCGTTTCCC
CCTGGAAGCTCCCTCGTGCGCTCTCCTGTTCCGACCCTGCCGCTTACCGGATACCTGTCCGCCTTTCTCCCTTCGGGAAG
CGTGGCGCTTTCTCATAGCTCACGCTGTAGGTATCTCAGTTCGGTGTAGGTCGTTCGCTCCAAGCTGGGCTGTGTGCACG
AACCCCCCGTTCAGCCCGACCGCTGCGCCTTATCCGGTAACTATCGTCTTGAGTCCAACCCGGTAAGACACGACTTATCG
CCACTGGCAGCAGCCACTGGTAACAGGATTAGCAGAGCGAGGTATGTAGGCGGTGCTACAGAGTTCTTGAAGTGGTGGCC
TAACTACGGCTACACTAGAAGAACAGTATTTGGTATCTGCGCTCTGCTGAAGCCAGTTACCTTCGGAAAAAGAGTTGGTA
GCTCTTGATCCGGCAAACAAACCACCGCTGGTAGCGGTGGTTTTTTTGTTTGCAAGCAGCAGATTACGCGCAGAAAAAAA
GGATCTCAAGAAGATCCTTTGATCTTTTCTACGGGGTCTGACGCTCAGTGGAACGAAAACTCACGTTAAGGGATTTTGGT
CATGAGATTATCAAAAAGGATCTTCACCTAGATCCTTTTAAATTAAAAATGAAGTTTTAAATCAATCTAAAGTATATATG
AGTAAACTTGGTCTGACAGTTACCAATGCTTAATCAGTGAGGCACCTATCTCAGCGATCTGTCTATTTCGTTCATCCATA
GTTGCCTGACTCCCCGTCGTGTAGATAACTACGATACGGGAGGGCTTACCATCTGGCCCCAGTGCTGCAATGATACCGCG
AGACCCACGCTCACCGGCTCCAGATTTATCAGCAATAAACCAGCCAGCCGGAAGGGCCGAGCGCAGAAGTGGTCCTGCAA
CTTTATCCGCCTCCATCCAGTCTATTAATTGTTGCCGGGAAGCTAGAGTAAGTAGTTCGCCAGTTAATAGTTTGCGCAAC
GTTGTTGCCATTGCTACAGGCATCGTGGTGTCACGCTCGTCGTTTGGTATGGCTTCATTCAGCTCCGGTTCCCAACGATC
AAGGCGAGTTACATGATCCCCCATGTTGTGCAAAAAAGCGGTTAGCTCCTTCGGTCCTCCGATCGTTGTCAGAAGTAAGT
TGGCCGCAGTGTTATCACTCATGGTTATGGCAGCACTGCATAATTCTCTTACTGTCATGCCATCCGTAAGATGCTTTTCT
GTGACTGGTGAGTACTCAACCAAGTCATTCTGAGAATAGTGTATGCGGCGACCGAGTTGCTCTTGCCCGGCGTCAATACG
GGATAATACCGCGCCACATAGCAGAACTTTAAAAGTGCTCATCATTGGAAAACGTTCTTCGGGGCAAAACTCTCAAGGAT
CTTACCGCTGTTGAGATCCAGTTCGATGTAACCCACTCGTGCACCCAACTGATCTTCAGCATCTTTTACTTTCACCAGCG
TTTCTGGGTGAGCAAAAACAGGAAGGCAAAATGCCGCAAAAAAGGGAATAAGGGCGACACGGAAATGTTGAATACTCATA
CTCTTCCTTTTTCAATATTATTGAAGCATTTATCAGGGTTATTGTCTCATGAGCGGATACATATTTGAATGTATTTAGAA
AAATAAACAAATAGGGGTTCCGCGCACATTTCCCCGAAAAGTGCCACCTGACGTCTAAGAAACCATTATTATCATGACAT
TAACCTATAAAAATAGGCGTATCACGAGGCCCTTTCGTCTCGCGCGTTTCGGTGATGACGGTGAAAACCTCTGACACATG
CAGCTCCCGGAGACGGTCACAGCTTGTCTGTAAGCGGATGCCGGGAGCAGACAAGCCCGTCAGGGCGCGTCAGCGGGTGT
TGGCGGGTGTCGGGGCTGGCTTAACTATGCGGCATCAGAGCAGATTGTACTGAGAGTGCACC
[SEQ ID NO: 76]
attggccattgcatacgttgtatccatatcataatatgtacatttatattggctcatgtccaacattaccgccatgttga
cattgattattgactagttattaatagtaatcaattacggggtcattagttcatagcccatatatggagttccgcgttac
ataacttacggtaaatggcccgcctggctgaccgcccaacgacccccgcccattgacgtcaataatgacgtatgttccca
tagtaacgccaatagggactttccattgacgtcaatgggtggagtatttacggtaaactgcccacttggcagtacatcaa
gtgtatcatatgccaagtacgccccctattgacgtcaatgacggtaaatggcccgcctggcattatgcccagtacatgac
cttatgggactttcctacttggcagtacatctacgtattagtcatcgctattaccatggtgatgcggttttggcagtaca
tcaatgggcgtggatagcggtttgactcacggggatttccaagtctccaccccattgacgtcaatgggagtttgttttgg
caccaaaatcaacgggactttccaaaatgtcgtaacaactccgccccattgacgcaaatgggcggtaggcgtgtacggtg
ggaggtctatataagcagagctcgtttagtgaaccggggtctctctggttagaccagatctgagcctgggagctctctgg
ctaactagggaacccactgcttaagcctcaataaagcttgccttgagtgcttcaagtagtgtgtgcccgtctgttgtgtg
actctggtaactagagatccctcagacccttttagtcagtgtggaaaatctctagcagtggcgcccgaacagggacttga
aagcgaaagggaaaccagaggagctctctcgacgcaggactcggcttgctgaagcgcgcacggcaagaggcgaggggcgg
cgactggtgagtacgccaaaaattttgactagcggaggctagaaggagagagacgggtgcgagagcgtcagtattaagcg
ggggtgaataagatcgcgatgggaaaaaattcggttaaggccagggggaaagaaaaaatataaattaaaacatatagtat
gggcaagcagggagctagaacgattcgcagttaatcctggcctgttagaaacatcagaaggctgtagacaaatactggga
cagctacaaccatcccttcagacaggatcagaagaacttagatcattatataatacagtagcaaccctctattgtgtgca
tcaaaggatagagataaaagacaccaaggaagctttagacaagatagaggaagagcaaaacaaaagtaagaccaccgcac
agcaagcggccgctgatcttcagacctggaggaggagatatgagggacaattggagaagtgaattatataaatataaagt
agtaaaaattgaaccattaggagtagcacccaccaaggcaaagagaagagtggtgcagagagaaaaaagagcagtgggaa
taggagctttgttccttgggttcttgggagcagcaggaagcactatgggcgcagcgtcaatgacgctgacggtacaggcc
agacaattattgtctggtatagtgcagcagcagaacaatttgctgagggctattgaggcgcaacagcatctgttgcaact
cacagtctggggcatcaagcagctccaggcaagaatcctggctgtggaaagatacctaaaggatcaacagctcctgggga
tttggggttgctctggaaaactcatttgcaccactgctgtgccttggaatgctagttggagtaataaatctctggaacag
atttggaatcacacgacctggatggagtgggacagagaaattaacaattacacaagcttaatacactccttaattgaaga
atcgcaaaaccagcaagaaaagaatgaacaagaattattggaattagataaatgggcaagtttgtggaattggtttaaca
taacaaattggctgtggtatataaaattattcataatgatagtaggaggcttggtaggtttaagaatagtttttgctgta
ctttctatagtgaatagagttaggcagggatattcaccattatcgtttcagacccacctcccaaccccgaggggacccga
caggcccgaaggaatagaagaagaaggtggagagagagacagagacagatccattcgattagtgaacggatctcgacggt
atcgttttaaaagaaaaggggggattggggggtacagtgcaggggaaagaatagtagacataatagcaacagacatacaa
actaaagaattacaaaaacaaattacaaaaattcaaaattttatcggcgtgttgggggtggaccatcctctaggtattga
ataagaaaaatgaagttaaggtggttgatggtaacactatgctaataactgcagagccagaagcaccataagggacatga
taagggagccagcagacctctgatctcttcctgaatgctaatcttaaacatcctgaggaagaatgggacttccatttggg
gtgggcctatgatagggtaataagacagtagtgaatatcaagctacaaaaagccccctttcaaattcttctcagtcctaa
cttttcatactaagcccagtccttccaaagcagactgtgaaagagtgatagttccgggagactagcactgcagattccgg
gtcactgtgagtgggggaggcagggaagaagggctcacaggacagtcaaaccatgccccctgtttttccttcttcaagta
gacctctataagacaacagagacaactaaggctgagtggccaggcgaggagaaaccatctcgccgtaaaacatggaagga
acacttcaggggaaaggtggtatctctaagcaagagaactgagtggagtcaaggctgagagatgcaggataagcaaatgg
gtagtgaaaagacattcatgaggacagctaaaacaataagtaatgtaaaatacagcatagcaaaactttaacctccaaat
caagcctctacttgaatccttttctgagggatgaataaggcataggcatcaggggctgttgccaatgtgcattagctgtt
tgcagcctcaccttctttcatggagtttaagatatagtgtattttcccaaggtttgaactagctcttcatttctttatgt
tttaaatgcactgacctcccacattccctttttagtaaaatattcagaaataatttaaatacatcattgcaatgaaaata
aatgttttttattaggcagaatccagatgctcaaggcccttcataatatcccccagtttagtagttggacttagggaaca
aaggaacctttaatagaaattggacagcaagaaagcgagcttagtgatacttgtgggccagggcattagccacaccagcc
accactttctgataggcagcctgcactggtggggtgAATTCTTTGCCAAAGTGATGGGCCAGCACACAGACCAGCACGTT
GCCCAGGAGCTGTGGGAGGAAGATAAGAGGTATGAACATGATTAGCAAAAGGGCCTAGCTTGGACTCAGAATAATCCAGC
CTTATCCCAACCATAAAATAAAAGCAGAATGGTAGCTGGATTGTAGCTGCTATTAGCAATATGAAACCTCTTACATCAGT
TACAATTTATATGCAGAAATATTTATATGCAGAAATATTGCTATTGCCTTAACCCAGAAATTATCACTGTTATTCTTTAG
AATGGTGCAAAGAGGCATGATACATTGTATCATTATTGCCCTGAAAGAAAGAGATTAGGGAAAGTATTAGAAATAAGATA
AACAAAAAAGTATATTAAAAGAAGAAAGCATTTTTTAAAATTACAAATGCAAAATTACCCTGATTTGGTCAATATGTGTA
CCCTGTTACTTCTCCCCTTCCTATGACATGAACTTAACCATAGAAAAGAAGGGGAAAGAAAACATCAAGGGTCCCATAGA
CTCACCCTGAAGTTCTCAGGATCCACGTGCAGCTTGTCACAGTGCAGCTCACTCAGctgGGCAAAGGTGCCCTTGAGGTT
GTCCAGGTGAGCCAGGCCATCACTAAAGGCACCGAGCACTTTCTTGCCATGAGCCTTCACCTTAGGGTTGCCCATAACAG
CATCAGGAGTGGACAGATCCCCAAAGGACTCAAAGAACCTCTGGGTCCAAGGGTAGACCACCAGCAGCCTAAGGGTGGGA
AAATAGACCAATAGGCAGAGAGAGTCAGTGCCTATCAGAAACCCAAGAGTCTTCTCTGTCTCCACATGCCCAGTTTCTAT
TGGTCTCCTTAAACCTGTCTTGTAACCTTGATACCAACCTGCCCAGGGCCTCACCACCAACTTCATCCACGTTCACCTTG
CCCCACAGGGCAGTAACGGCAGACTTCTCCTCAGGAGTCAGGTGCACCATGGTGTCTGTTTGAGGTTGCTAGTGAACACA
GTTGTGTCAGAAGCAAATGTAAGCAATAGATGGCTCTGCCCTGACTTTTATGCCCAGCCCTGGCTCCTGCCCTCCCTGCT
CCTGGGAGTAGATTGGCCAACCctagggtgtggctccacagggtgaggtctaagtgatgacagccgtacctgtccttggc
tcttctggcactggcttaggagttggacttcaaaccctcagccctccctctaagatatatctcttggccccataccatca
gtacaaattgctactaaaaacatcctcctttgcaagtgtatttacCACCCTTTTCCGGCACGCAGATAGTCAATATCTTC
AGCGTCCCCAAGGCCTGCAAGGGTGGGGCCCCATATCTGGAAGTCCCAGGCGGAGCTGGGAGTTGGTCAAGTCTGGGCTG
TGGGGGCAGGGAGTGCTGGGGGATGGacgcgtcgacggtaccgttaacgatcttagccactttttaaaagaaaagggggg
actggaagggctaattcactcccaacgaagacaagatatcctgCTAGTCCTTCCTTTCTAAATGACGAGAGAGACAGAAG
AATTCTTCAAGGTTAGTGTGTCCAGCATGCAACCTTTCCTTCCTGGATGAGCATCCCTGGAGTAGGAGAGCCAGCCTGCC
TCCTGCGCTGGCACAGAGCCCGGTTCCCTAGACAACTGCCTCTCCAAATCTGATGTCCAGCGCCACCTGGTGTCCACATC
AAGCAGACACAATTAATAGTCAACCTGTTCAGGAAAACTGTGAGGGGGAAAAAAAAGAAAGAGGATTTATGAAGGGAAAA
GAAAGTTTAGAGGATATGCCACGATTGGctagcagctgctttttgcctgtactgggtctctctggttagaccagatctga
gcctgggagctctctggctaactagggaacccactgcttaagcctcaataaagcttgccttgagtgcttcatccggAATC
AACCTCTGGATTACAAAATTTGTGAAAGATTGACTGGTATTCTTAACTATGTTGCTCCTTTTACGCTATGTGGATACGCT
GCTTTAATGCCTTTGTATCATGCTATTGCTTCCCGTATGGCTTTCATTTTCTCCTCCTTGTATAAATCCTGGTTGCTGTC
TCTTTATGAGGAGTTGTGGCCCGTTGTCAGGCAACGTGGCGTGGTGTGCACTGTGTTTGCTGACGCAACCCCCACTGGTT
GGGGCATTGCCACCACCTGTCAGCTCCTTTCCGGGACTTTCGCTTTCCCCCTCCCTATTGCCACGGCGGAACTCATCGCC
GCCTGCCTTGCCCGCTGCTGGACAGGGGCTCGGCTGTTGGGCACTGACAATTCCGTGGTGTTGTCGGGGAAGCTGACGTC
CTTTCCATGGCTGCTCGCCTGTGTTGCCACCTGGATTCTGCGCGGGACGTCCTTCTGCTACGTCCCTTCGGCCCTCAATC
CAGCGGACCTTCCTTCCCGCGGCCTGCTGCCGGCTCTGCGGCCTCTTCCGCGTCTTCGCCTTCGCCCTCAGACGAGTCGG
ATCTCCCTTTGGGCCGCCTCCCCGCCTGtccggtAGCTTGCCAGCCTCGACTGTGCCTTCTAGTTGCCAGCCGTCTGTTG
TTTGCCCCTCCCCCGTGCCTTCCTTGACCCTGGAAGGTGCCACTCCCACTGTCCTTTCCTAATAAAATGAGGAAATTGCA
TCGCATTGTCTGAGTAGGTGTCATTCTATTCTGGGGGGTGGGGTGGGGCAGGACAGCAAGGGGGAGGATTGGGAAGACAA
TAGCAGGCATGcaagcttggcgtaatcatggtcatagctgtttcctgtgtgaaattgttatccgctcacaattccacaca
acatacgagccggaagcataaagtgtaaagcctggggtgcctaatgagtgagctaactcacattaattgcgttgcgctca
ctgcccgctttccagtcgggaaacctgtcgtgccagctgcattaatgaatcggccaacgcgcggggagaggcggtttgcg
tattgggcgctcttccgcttcctcgctcactgactcgctgcgctcggtcgttcggctgcggcgagcggtatcagctcact
caaaggcggtaatacggttatccacagaatcaggggataacgcaggaaagaacatgtgagcaaaaggccagcaaaaggcc
aggaaccgtaaaaaggccgcgttgctggcgtttttccataggctccgcccccctgacgagcatcacaaaaatcgacgctc
aagtcagaggtggcgaaacccgacaggactataaagataccaggcgtttccccctggaagctccctcgtgcgctctcctg
ttccgaccctgccgcttaccggatacctgtccgcctttctcccttcgggaagcgtggcgctttctcatagctcacgctgt
aggtatctcagttcggtgtaggtcgttcgctccaagctgggctgtgtgcacgaaccccccgttcagcccgaccgctgcgc
cttatccggtaactatcgtcttgagtccaacccggtaagacacgacttatcgccactggcagcagccactggtaacagga
ttagcagagcgaggtatgtaggcggtgctacagagttcttgaagtggtggcctaactacggctacactagaagaacagta
tttggtatctgcgctctgctgaagccagttaccttcggaaaaagagttggtagctcttgatccggcaaacaaaccaccgc
tggtagcggtggtttttttgtttgcaagcagcagattacgcgcagaaaaaaaggatctcaagaagatcctttgatctttt
ctacggggtctgacgctcagtggaacgaaaactcacgttaagggattttggtcatgagattatcaaaaaggatcttcacc
tagatccttttaaattaaaaatgaagttttaaatcaatctaaagtatatatgagtaaacttggtctgacagttaccaatg
cttaatcagtgaggcacctatctcagcgatctgtctatttcgttcatccatagttgcctgactccccgtcgtgtagataa
ctacgatacgggagggcttaccatctggccccagtgctgcaatgataccgcgagacccacgctcaccggctccagattta
tcagcaataaaccagccagccggaagggccgagcgcagaagtggtcctgcaactttatccgcctccatccagtctattaa
ttgttgccgggaagctagagtaagtagttcgccagttaatagtttgcgcaacgttgttgccattgctacaggcatcgtgg
tgtcacgctcgtcgtttggtatggcttcattcagctccggttcccaacgatcaaggcgagttacatgatcccccatgttg
tgcaaaaaagcggttagctccttcggtcctccgatcgttgtcagaagtaagttggccgcagtgttatcactcatggttat
ggcagcactgcataattctcttactgtcatgccatccgtaagatgcttttctgtgactggtgagtactcaaccaagtcat
tctgagaatagtgtatgcggcgaccgagttgctcttgcccggcgtcaatacgggataataccgcgccacatagcagaact
ttaaaagtgctcatcattggaaaacgttcttcggggcaaaactctcaaggatcttaccgctgttgagatccagttcgatg
taacccactcgtgcacccaactgatcttcagcatcttttactttcaccagcgtttctgggtgagcaaaaacaggaaggca
aaatgccgcaaaaaagggaataagggcgacacggaaatgttgaatactcatactcttcctttttcaatattattgaagca
tttatcagggttattgtctcatgagcggatacatatttgaatgtatttagaaaaataaacaaataggggttccgcgcaca
tttccccgaaaagtgccacctgacgtctaagaaaccattattatcatgacattaacctataaaaataggcgtatcacgag
gccctttcgtctcgcgcgtttcggtgatgacggtgaaaacctctgacacatgcagctcccggagacggtcacagcttgtc
tgtaagcggatgccgggagcagacaagcccgtcagggcgcgtcagcgggtgttggcgggtgtcggggctggcttaactat
gcggcatcagagcagattgtactgagagtgcacc
[SEQ ID NO: 77]
attggccattgcatacgttgtatccatatcataatatgtacatttatattggctcatgtccaacattaccgccatgttga
cattgattattgactagttattaatagtaatcaattacggggtcattagttcatagcccatatatggagttccgcgttac
ataacttacggtaaatggcccgcctggctgaccgcccaacgacccccgcccattgacgtcaataatgacgtatgttccca
tagtaacgccaatagggactttccattgacgtcaatgggtggagtatttacggtaaactgcccacttggcagtacatcaa
gtgtatcatatgccaagtacgccccctattgacgtcaatgacggtaaatggcccgcctggcattatgcccagtacatgac
cttatgggactttcctacttggcagtacatctacgtattagtcatcgctattaccatggtgatgcggttttggcagtaca
tcaatgggcgtggatagcggtttgactcacggggatttccaagtctccaccccattgacgtcaatgggagtttgttttgg
caccaaaatcaacgggactttccaaaatgtcgtaacaactccgccccattgacgcaaatgggcggtaggcgtgtacggtg
ggaggtctatataagcagagctcgtttagtgaaccggggtctctctggttagaccagatctgagcctgggagctctctgg
ctaactagggaacccactgcttaagcctcaataaagcttgccttgagtgcttcaagtagtgtgtgcccgtctgttgtgtg
actctggtaactagagatccctcagacccttttagtcagtgtggaaaatctctagcagtggcgcccgaacagggacttga
aagcgaaagggaaaccagaggagctctctcgacgcaggactcggcttgctgaagcgcgcacggcaagaggcgaggggcgg
cgactggtgagtacgccaaaaattttgactagcggaggctagaaggagagagacgggtgcgagagcgtcagtattaagcg
ggggtgaataagatcgcgatgggaaaaaattcggttaaggccagggggaaagaaaaaatataaattaaaacatatagtat
gggcaagcagggagctagaacgattcgcagttaatcctggcctgttagaaacatcagaaggctgtagacaaatactggga
cagctacaaccatcccttcagacaggatcagaagaacttagatcattatataatacagtagcaaccctctattgtgtgca
tcaaaggatagagataaaagacaccaaggaagctttagacaagatagaggaagagcaaaacaaaagtaagaccaccgcac
agcaagcggccgctgatcttcagacctggaggaggagatatgagggacaattggagaagtgaattatataaatataaagt
agtaaaaattgaaccattaggagtagcacccaccaaggcaaagagaagagtggtgcagagagaaaaaagagcagtgggaa
taggagctttgttccttgggttcttgggagcagcaggaagcactatgggcgcagcgtcaatgacgctgacggtacaggcc
agacaattattgtctggtatagtgcagcagcagaacaatttgctgagggctattgaggcgcaacagcatctgttgcaact
cacagtctggggcatcaagcagctccaggcaagaatcctggctgtggaaagatacctaaaggatcaacagctcctgggga
tttggggttgctctggaaaactcatttgcaccactgctgtgccttggaatgctagttggagtaataaatctctggaacag
atttggaatcacacgacctggatggagtgggacagagaaattaacaattacacaagcttaatacactccttaattgaaga
atcgcaaaaccagcaagaaaagaatgaacaagaattattggaattagataaatgggcaagtttgtggaattggtttaaca
taacaaattggctgtggtatataaaattattcataatgatagtaggaggcttggtaggtttaagaatagtttttgctgta
ctttctatagtgaatagagttaggcagggatattcaccattatcgtttcagacccacctcccaaccccgaggggacccga
caggcccgaaggaatagaagaagaaggtggagagagagacagagacagatccattcgattagtgaacggatctcgacggt
atcgttttaaaagaaaaggggggattggggggtacagtgcaggggaaagaatagtagacataatagcaacagacatacaa
actaaagaattacaaaaacaaattacaaaaattcaaaattttatcggcgtgttgggggtggaccatcctctaggtattga
ataagaaaaatgaagttaaggtggttgatggtaacactatgctaataactgcagagccagaagcaccataagggacatga
taagggagccagcagacctctgatctcttcctgaatgctaatcttaaacatcctgaggaagaatgggacttccatttggg
gtgggcctatgatagggtaataagacagtagtgaatatcaagctacaaaaagccccctttcaaattcttctcagtcctaa
cttttcatactaagcccagtccttccaaagcagactgtgaaagagtgatagttccgggagactagcactgcagattccgg
gtcactgtgagtgggggaggcagggaagaagggctcacaggacagtcaaaccatgccccctgtttttccttcttcaagta
gacctctataagacaacagagacaactaaggctgagtggccaggcgaggagaaaccatctcgccgtaaaacatggaagga
acacttcaggggaaaggtggtatctctaagcaagagaactgagtggagtcaaggctgagagatgcaggataagcaaatgg
gtagtgaaaagacattcatgaggacagctaaaacaataagtaatgtaaaatacagcatagcaaaactttaacctccaaat
caagcctctacttgaatccttttctgagggatgaataaggcataggcatcaggggctgttgccaatgtgcattagctgtt
tgcagcctcaccttctttcatggagtttaagatatagtgtattttcccaaggtttgaactagctcttcatttctttatgt
tttaaatgcactgacctcccacattccctttttagtaaaatattcagaaataatttaaatacatcattgcaatgaaaata
aatgttttttattaggcagaatccagatgctcaaggcccttcataatatcccccagtttagtagttggacttagggaaca
aaggaacctttaatagaaattggacagcaagaaagcgagcttagtgatacttgtgggccagggcattagccacaccagcc
accactttctgataggcagcctgcactggtggggtgAATTCTTTGCCAAAGTGATGGGCCAGCACACAGACCAGCACGTT
GCCCAGGAGCTGTGGGAGGAAGATAAGAGGTATGAACATGATTAGCAAAAGGGCCTAGCTTGGACTCAGAATAATCCAGC
CTTATCCCAACCATAAAATAAAAGCAGAATGGTAGCTGGATTGTAGCTGCTATTAGCAATATGAAACCTCTTACATCAGT
TACAATTTATATGCAGAAATATTTATATGCAGAAATATTGCTATTGCCTTAACCCAGAAATTATCACTGTTATTCTTTAG
AATGGTGCAAAGAGGCATGATACATTGTATCATTATTGCCCTGAAAGAAAGAGATTAGGGAAAGTATTAGAAATAAGATA
AACAAAAAAGTATATTAAAAGAAGAAAGCATTTTTTAAAATTACAAATGCAAAATTACCCTGATTTGGTCAATATGTGTA
CCCTGTTACTTCTCCCCTTCCTATGACATGAACTTAACCATAGAAAAGAAGGGGAAAGAAAACATCAAGGGTCCCATAGA
CTCACCCTGAAGTTCTCAGGATCCACGTGCAGCTTGTCACAGTGCAGCTCACTCAGctgGGCAAAGGTGCCCTTGAGGTT
GTCCAGGTGAGCCAGGCCATCACTAAAGGCACCGAGCACTTTCTTGCCATGAGCCTTCACCTTAGGGTTGCCCATAACAG
CATCAGGAGTGGACAGATCCCCAAAGGACTCAAAGAACCTCTGGGTCCAAGGGTAGACCACCAGCAGCCTAAGGGTGGGA
AAATAGACCAATAGGCAGAGAGAGTCAGTGCCTATCAGAAACCCAAGAGTCTTCTCTGTCTCCACATGCCCAGTTTCTAT
TGGTCTCCTTAAACCTGTCTTGTAACCTTGATACCAACCTGCCCAGGGCCTCACCACCAACTTCATCCACGTTCACCTTG
CCCCACAGGGCAGTAACGGCAGACTTCTCCTCAGGAGTCAGGTGCACCATGGTGTCTGTTTGAGGTTGCTAGTGAACACA
GTTGTGTCAGAAGCAAATGTAAGCAATAGATGGCTCTGCCCTGACTTTTATGCCCAGCCCTGGCTCCTGCCCTCCCTGCT
CCTGGGAGTAGATTGGCCAACCctagggtgtggctccacagggtgaggtctaagtgatgacagccgtacctgtccttggc
tcttctggcactggcttaggagttggacttcaaaccctcagccctccctctaagatatatctcttggccccataccatca
gtacaaattgctactaaaaacatcctcctttgcaagtgtatttacCACCCTTTTCCGGCACGCAGATAGTCAATATCTTC
AGCGTCCCCAAGGCCTGCAAGGGTGGGGCCCCATATCTGGAAGTCCCAGGCGGAGCTGGGAGTTGGTCAAGTCTGGGCTG
TGGGGGCAGGGAGTGCTGGGGGATGGacgcgtatctgcggccgcctatctgtaccactagtctcgagaagctttcatcaa
aaaaagtctaaccagctgcattcgactttgactgcagcagctggttagaaggttctactggaggagggtcccagcccatt
gctaaattaacatcaggctctgagactggcagtatatctctaacagtggttgatgctatcttctggaacttgcctgctac
attgagaccactgacccatacataggaagcccatagctctgtcctgaactgttaggccactggtccagagagtgtgcatc
tcctttgatcctcataataaccctatgagatagacacaattattactcttactttatagatgatgatcctgaaaacatag
gagtcaaggcacttgcccctagctgggggtataggggagcagtcccatgtagtagtagaatgaaaaatgctgctatgctg
tgcctcccccacctttcccatgtctgccctctactcatggtctatctctcctggctcctgggagtcatggactccaccca
gcaccaccaacctgacctaaccacctatctgagcctgccagcctataacccatctgggccctgatagctggtggccagcc
ctgaccccaccccaccctccctggaacctctgatagacacatctggcacaccagctcgcaaagtcaccgtgagggtcttg
tgtttgctgagtcaaaattccttgaaatccaagtccttagagactcctgctcccaaatttacagtcatagacttcttcat
ggctgtctcctttatccacagaatgattcctttgcttcattgccccatccatctgatcctcctcatcagtgcagcacagg
gcccatgagcagtagctgcagagtctcacataggtctggcactgcctctgacatgtccgaccttaggcaaatgcttgact
cttctgagctcagtcttgtcatggcaaaacaaagataataatagtgtttttttatggagttagcgtgaggatggaaaaca
atagcaaaattgattagactataaaaggtctcaacaaatagtagtagattttatcatccattaatccttccctctcctct
cttactcatcccatcacgtatgcctcttaattttcccttacctataataagagttattcctcttattatattcttcttat
agtgattctggatatcaaagtgggaatgaggggcaggccactaacgaagaagatgtttctcaaagaagccattctcccca
catagatcatctcagcagggttcaggaagataaaggaggatcaaggtcgaaggtaggaactaaggaagaacactgggcaa
gtgacgcgtcgacggtaccgttaacgatcttagccactttttaaaagaaaaggggggactggaagggctaattcactccc
aacgaagacaagatatcctgCTAGTCCTTCCTTTCTAAATGACGAGAGAGACAGAAGAATTCTTCAAGGTTAGTGTGTCC
AGCATGCAACCTTTCCTTCCTGGATGAGCATCCCTGGAGTAGGAGAGCCAGCCTGCCTCCTGCGCTGGCACAGAGCCCGG
TTCCCTAGACAACTGCCTCTCCAAATCTGATGTCCAGCGCCACCTGGTGTCCACATCAAGCAGACACAATTAATAGTCAA
CCTGTTCAGGAAAACTGTGAGGGGGAAAAAAAAGAAAGAGGATTTATGAAGGGAAAAGAAAGTTTAGAGGATATGCCACG
ATTGGctagcagctgctttttgcctgtactgggtctctctggttagaccagatctgagcctgggagctctctggctaact
agggaacccactgcttaagcctcaataaagcttgccttgagtgcttcatccggAATCAACCTCTGGATTACAAAATTTGT
GAAAGATTGACTGGTATTCTTAACTATGTTGCTCCTTTTACGCTATGTGGATACGCTGCTTTAATGCCTTTGTATCATGC
TATTGCTTCCCGTATGGCTTTCATTTTCTCCTCCTTGTATAAATCCTGGTTGCTGTCTCTTTATGAGGAGTTGTGGCCCG
TTGTCAGGCAACGTGGCGTGGTGTGCACTGTGTTTGCTGACGCAACCCCCACTGGTTGGGGCATTGCCACCACCTGTCAG
CTCCTTTCCGGGACTTTCGCTTTCCCCCTCCCTATTGCCACGGCGGAACTCATCGCCGCCTGCCTTGCCCGCTGCTGGAC
AGGGGCTCGGCTGTTGGGCACTGACAATTCCGTGGTGTTGTCGGGGAAGCTGACGTCCTTTCCATGGCTGCTCGCCTGTG
TTGCCACCTGGATTCTGCGCGGGACGTCCTTCTGCTACGTCCCTTCGGCCCTCAATCCAGCGGACCTTCCTTCCCGCGGC
CTGCTGCCGGCTCTGCGGCCTCTTCCGCGTCTTCGCCTTCGCCCTCAGACGAGTCGGATCTCCCTTTGGGCCGCCTCCCC
GCCTGtccggtAGCTTGCCAGCCTCGACTGTGCCTTCTAGTTGCCAGCCGTCTGTTGTTTGCCCCTCCCCCGTGCCTTCC
TTGACCCTGGAAGGTGCCACTCCCACTGTCCTTTCCTAATAAAATGAGGAAATTGCATCGCATTGTCTGAGTAGGTGTCA
TTCTATTCTGGGGGGTGGGGTGGGGCAGGACAGCAAGGGGGAGGATTGGGAAGACAATAGCAGGCATGcaagcttggcgt
aatcatggtcatagctgtttcctgtgtgaaattgttatccgctcacaattccacacaacatacgagccggaagcataaag
tgtaaagcctggggtgcctaatgagtgagctaactcacattaattgcgttgcgctcactgcccgctttccagtcgggaaa
cctgtcgtgccagctgcattaatgaatcggccaacgcgcggggagaggcggtttgcgtattgggcgctcttccgcttcct
cgctcactgactcgctgcgctcggtcgttcggctgcggcgagcggtatcagctcactcaaaggcggtaatacggttatcc
acagaatcaggggataacgcaggaaagaacatgtgagcaaaaggccagcaaaaggccaggaaccgtaaaaaggccgcgtt
gctggcgtttttccataggctccgcccccctgacgagcatcacaaaaatcgacgctcaagtcagaggtggcgaaacccga
caggactataaagataccaggcgtttccccctggaagctccctcgtgcgctctcctgttccgaccctgccgcttaccgga
tacctgtccgcctttctcccttcgggaagcgtggcgctttctcatagctcacgctgtaggtatctcagttcggtgtaggt
cgttcgctccaagctgggctgtgtgcacgaaccccccgttcagcccgaccgctgcgccttatccggtaactatcgtcttg
agtccaacccggtaagacacgacttatcgccactggcagcagccactggtaacaggattagcagagcgaggtatgtaggc
ggtgctacagagttcttgaagtggtggcctaactacggctacactagaagaacagtatttggtatctgcgctctgctgaa
gccagttaccttcggaaaaagagttggtagctcttgatccggcaaacaaaccaccgctggtagcggtggtttttttgttt
gcaagcagcagattacgcgcagaaaaaaaggatctcaagaagatcctttgatcttttctacggggtctgacgctcagtgg
aacgaaaactcacgttaagggattttggtcatgagattatcaaaaaggatcttcacctagatccttttaaattaaaaatg
aagttttaaatcaatctaaagtatatatgagtaaacttggtctgacagttaccaatgcttaatcagtgaggcacctatct
cagcgatctgtctatttcgttcatccatagttgcctgactccccgtcgtgtagataactacgatacgggagggcttacca
tctggccccagtgctgcaatgataccgcgagacccacgctcaccggctccagatttatcagcaataaaccagccagccgg
aagggccgagcgcagaagtggtcctgcaactttatccgcctccatccagtctattaattgttgccgggaagctagagtaa
gtagttcgccagttaatagtttgcgcaacgttgttgccattgctacaggcatcgtggtgtcacgctcgtcgtttggtatg
gcttcattcagctccggttcccaacgatcaaggcgagttacatgatcccccatgttgtgcaaaaaagcggttagctcctt
cggtcctccgatcgttgtcagaagtaagttggccgcagtgttatcactcatggttatggcagcactgcataattctctta
ctgtcatgccatccgtaagatgcttttctgtgactggtgagtactcaaccaagtcattctgagaatagtgtatgcggcga
ccgagttgctcttgcccggcgtcaatacgggataataccgcgccacatagcagaactttaaaagtgctcatcattggaaa
acgttcttcggggcaaaactctcaaggatcttaccgctgttgagatccagttcgatgtaacccactcgtgcacccaactg
atcttcagcatcttttactttcaccagcgtttctgggtgagcaaaaacaggaaggcaaaatgccgcaaaaaagggaataa
gggcgacacggaaatgttgaatactcatactcttcctttttcaatattattgaagcatttatcagggttattgtctcatg
agcggatacatatttgaatgtatttagaaaaataaacaaataggggttccgcgcacatttccccgaaaagtgccacctga
cgtctaagaaaccattattatcatgacattaacctataaaaataggcgtatcacgaggccctttcgtctcgcgcgtttcg
gtgatgacggtgaaaacctctgacacatgcagctcccggagacggtcacagcttgtctgtaagcggatgccgggagcaga
caagcccgtcagggcgcgtcagcgggtgttggcgggtgtcggggctggcttaactatgcggcatcagagcagattgtact
gagagtgcacc

These vectors were produced by transient triple transfection of the recombinant vector, pCMVAR8.9 and pMD.G into 293T cells. A total of 5×10⁶ 293T cells were seeded in 10-cm-diameter dishes 24 h prior to transfection in DMEM with 10% fetal bovine serum, penicillin (100 IU/ml), and streptomycin (100 μg/ml) in a 5% CO₂ incubator. A total of 20 μg of plasmid DNA was used for the transfection of one 10 cm dish: 3.5 μg of the envelope plasmid pMD.G, 6.5 μg of packaging plasmid, and 10 μg of transfer vector plasmid. The medium (10 ml) was replaced after 14 to 16 h; the conditioned medium was collected after another 24 h, cleared by low-speed centrifugation, and filtered through 0.45-μm-pore-size cellulose acetate filters. The pseudotyped virions were than concentrated by ultracentrifugation and resuspended for frozen down in aliquots for subsequent titration and stem cell transduction.

### Titration Assay

These vectors were produced by transient triple transfection of the recombinant vector, pCMVAR8.9 and pMD.G into 293T cells. A total of 5×10⁶ 293T cells were seeded in 10-cm-diameter dishes 24 h prior to transfection in DMEM with 10% fetal bovine serum, penicillin (100 IU/ml), and streptomycin (100 μg/ml) in a 5% CO₂ incubator. A total of 20 μg of plasmid DNA was used for the transfection of one 10 cm dish: 3.5 μg of the envelope plasmid pMD.G, 6.5 μg of packaging plasmid, and 10 μg of transfer vector plasmid. The medium (10 ml) was replaced after 14 to 16 h; the conditioned medium was collected after another 24 h, cleared by low-speed centrifugation, and filtered through 0.45-μm-pore-size cellulose acetate filters. The pseudotyped virions were than used directly for titration assays in Hela cells. Serial dilutions of un-concentrated virus were used to infect 20⁵ cells in a six-well plate in the presence of Polybrene (8 μg/ml). each dilution was tested in triplicate. The cells were kept in culture for no less than 15 days, harvested and genomic DNA were extracted, quantified and used to Vector Copy Number (VCN) quantification. Transducing activity was expressed in transducing units (TU). VCN analysis was performed by TaqMan PCR analysis using:

Gag-specific probe
(SEQ ID NO: 41)
5′-acagccttctgatgtttctaacaggccagg-3′
Gag-primer-Forward:
(SEQ ID NO: 42)
5′-ggagctagaacgattcgcagtt-3′
Gag-primer-Reverse:
(SEQ ID NO: 43)
5′-gttgtagctgtcccagtatttgtc-3′
Human Albumin Probe
(SEQ ID NO: 44)
5′-tgctgaaacattcaccttccatgcagt-3′
Human Albumin Forward
(SEQ ID NO: 45)
5′tgaaacatacgttcccaaagagttt-3′
Human Albumin Reverse
(SEQ ID NO: 46)
5′ctctccttctcagaaagtgtgcatat -3′

### Hematopoietic Stem Cell Transduction and Transplantation

Donor bone marrow was flushed from the femurs of 8- to 16-week-old male Hbb^{th3/+} thalassemic mice that had been injected intravenously (i.v.) 6 days earlier with 5-flurouracil (5-FU, Pharmacia; 150 mg kg⁻¹ body weight). Bone marrow cells were resuspended in serum-free medium, and supplemented with rmSCF (100 ng ml⁻¹), rmTPO (100 ng ml⁻¹), β-mercaptoethanol (55 μM; Sigma), L-glutamine (2 mM), pen/strep (10 IU ml⁻¹), and cultured for 18 h. Bone marrow cells were pelleted and resuspended in serum-free medium containing concentrated lentiviral supernatant at a multiplicity of infection (MOI) of 25 and supplemented with polybrene (8 μg ml⁻¹), L-glutamine (2 mM), pen/strep (100 IU ml⁻¹) and incubated for 8 h. Transduced bone marrow cells (5×10⁵)) were then i.v. injected into each of the irradiated female recipients to establish bone marrow chimaeras. Recipient mice (11- to 14-week-old C57/Hbb^{th3/+} mice) were irradiated with 10.5 Gy (split dose 2×5.25 Gy) on the day of transplantation.

### Post-Transplant Vector Studies

Peripheral Blood was collected periodically starting 5-6 weeks after hematopoietic stem cell transplantation. Blood samples were obtained by retro-orbital puncture under ether anesthesia, according to MSKCC animal protocol. Total hemoglobin levels, red cell counts, hematocrit levels, neutrophil counts, platelet counts, and reticulocyte counts were measured on a blood cell count analyzer.

VCN analysis: To evaluate the integrated vector copy number in peripheral blood, mouse genomic DNA was isolated, quantified and analyzed with multiplex real-time TaqMan PCR using following probes and primer:

Gag-specific probe
(SEQ ID NO: 41)
5′-acagccttctgatgtttctaacaggccagg-3′
Gag-primer-Forward
(SEQ ID NO: 42)
5′-ggagctagaacgattcgcagtt-3′
Gag-primer-Reverse
(SEQ ID NO: 43)
5′-gttgtagctgtcccagtatttgtc-3′
Mouse β-actin Probe
(SEQ ID NO: 47)
5′-tacgagggctatgctctccctcacgc-3′
Mouse β-actin-primer-Forward
(SEQ ID NO: 48)
5′-tcacccacactgtgcccat-3′
Mouse β-actin-primer-reverse
(SEQ ID NO: 49)
5′-agccaggtccagacgcag-3′

Hemoglobin assays: automated hemoglobin (Hb) quantification; based line anemia in Hbb^{th3/+} mice is 7.5-8.5 g/dL. Vector performance, i.e., protein output per vector copy, was determined as the ratio of gain in hemoglobin expression (i.e., correction of anemia), ΔHb g/dL, per vector copy number (VCN) measured in circulating blood cells.

### Results and Discussion

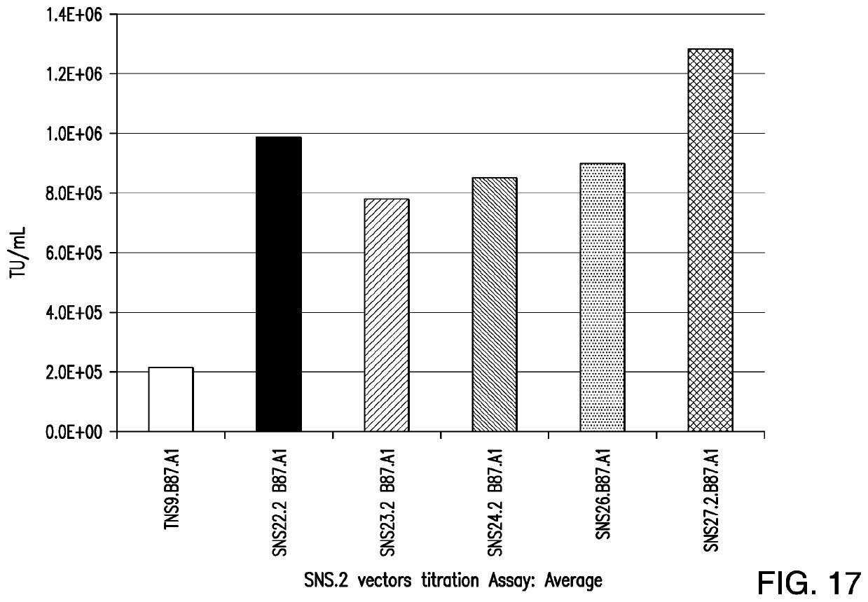

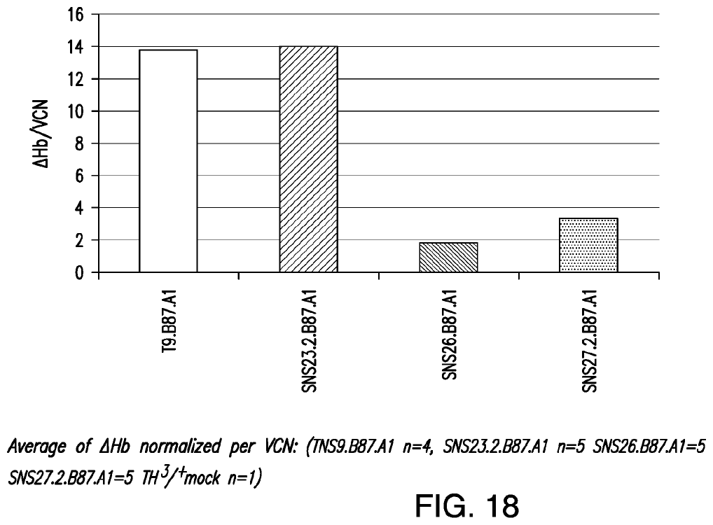

As shown in FIG. 17, the SNS22.2.B87.A1, SNS23.2.B87.A1, SNS24.2.B87.A1, SNS26.B87.A1 and SNS27.2.B87.A1 vectors achieved higher titer than the TNS9.B87.A1 vector. Moreover, as shown in FIG. 16 and Table 1, the multiple mutations and/or deletions introduced into the promoter, HS2, HS3, and HS4 regions of the β-globin LCR of SNS23.2.B87.A1 did not affect (e.g., did not reduce) globin expression in mice. Furthermore, vectors lacking HS2 or all LCR elements but including an BLV enhancer and flanked by the A1 insulator, e.g., SNS26.B87.A1 and SNS27.2.B87.A1, can effectively treat anemia in thalassemic mice. While the globin protein output per copy of the SNS26.B87.A1 vector or the SNS27.2.B87.A1 vector was less than that of the SNS23.2.B87.A1 vector (see FIG. 18 and Table1), SNS26.B87.A1 and SNS27.2.B87.A1 vectors were able to correct the disease with a higher vector copy number (see FIG. 16). The higher titer of the SNS26.B87.A1 and SNS27.2.B87.A1 vectors facilitated higher gene transfer, resulting in a higher vector copy number (VCN) and therapeutic activity.

As shown in FIG. 16, the expression of an incomplete or lacking LCR required 0.8 or higher copies of vector per cell, while in the case of SNS23.2.B87.A1, a VCN of 0.3 copies was sufficient to achieve correction of anemia

TABLE 1
Vector copy number (VCN) and Hb levels in long-term
hematopoietic chimeras. Total Hb level [g/dL] in peripheral
blood (PB) of chimeric mice. Representative data for week 6
after transplant is shown. ΔHb level was obtained by
subtracting Th3/+ hemoglobin (value = 9 g/dL) from
total Hb level for each animal tested. ΔHb/copy =
Correlation between delta(Δ)Hb and vector copy number.

| | | HGB | | ΔHGB/ |
|---|---|---|---|---|
| mouse ID | VCN | (g/dL) | Δ(HGB-9) | copy |
| TNS9.B87.A1 #1 | 0.2 | 11.3 | 2.3 | 11.5 |
| TNS9.B87.A1 #2 | 0.2 | 12.0 | 3.0 | 16.7 |
| TNS9.B87.A1 #3 | 0.2 | 8.5 | −0.5 | −2.5 |
| TNS9.B87.A1 #4 | 0.2 | 13.5 | 4.5 | 30.0 |
| SNS23.2.B87.A1 #1 | 0.3 | 13.1 | 4.1 | 15.8 |
| SNS23.2.B87.A1 #2 | 0.3 | 13.3 | 4.3 | 13.0 |
| SNS23.2.B87.A1 #3 | 0.3 | 13.2 | 4.2 | 14.5 |
| SNS23.2.B87.A1 #4 | 0.3 | 13.0 | 4.0 | 13.8 |
| SNS23.2.B87.A1 #5 | 0.3 | 13.2 | 4.2 | 13.5 |
| SNS26.B87.A1 #1 | 1.2 | 11.4 | 2.4 | 2.0 |
| SNS26.B87.A1 #2 | 1.2 | 11.9 | 2.9 | 2.3 |
| SNS26.B87.A1 #3 | 1.3 | 9.7 | 0.7 | 0.6 |
| SNS26.B87.A1 #4 | 1.7 | 11.9 | 2.9 | 1.8 |
| SNS26.B87.A1 #5 | 1.3 | 11.3 | 2.3 | 1.8 |
| SNS27.2.B87.A1 #1 | 0.8 | 11.7 | 2.7 | 3.3 |
| SNS27.2.B87.A1 #2 | 0.8 | 13.0 | 4.0 | 5.3 |
| SNS27.2.B87.A1 #3 | 0.9 | 11.5 | 2.5 | 2.7 |
| SNS27.2.B87.A1 #4 | 0.9 | 11.5 | 2.5 | 2.7 |
| SNS27.2.B87.A1 #5 | 0.7 | 11.3 | 2.3 | 3.2 |
| TH3/+ MOCK #1 | — | 7.5 | −1.5 | |

### Example 7: Evaluation of Globin Production at Different Time Points

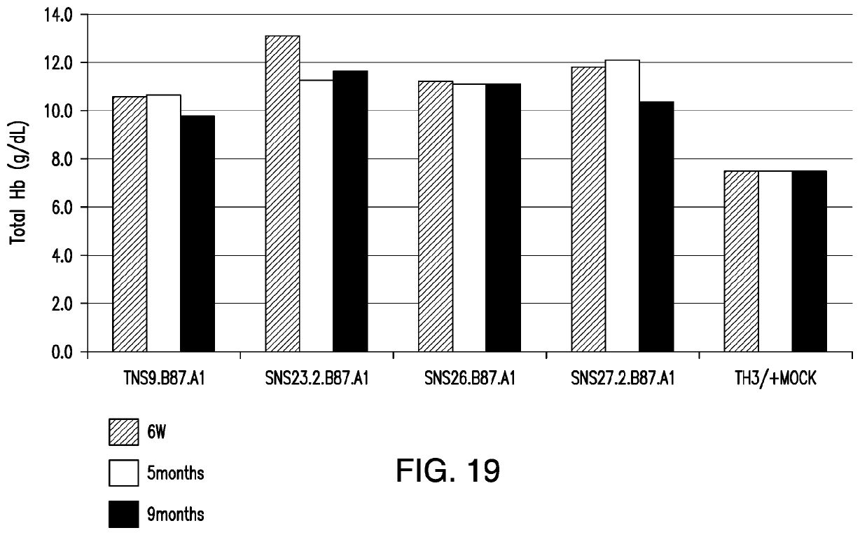

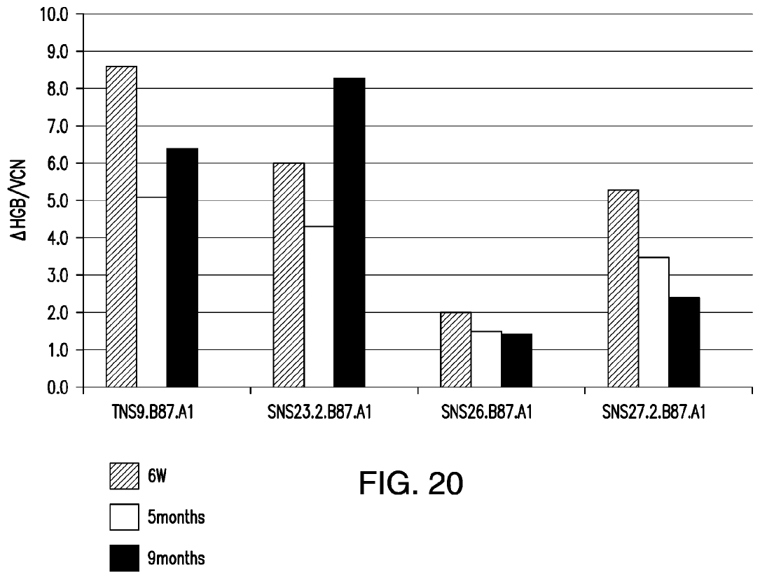

Additional experiments were conducted to measure the globin production at different time points in thalassemic mice transfected with vectors disclosed herein. The methods used in Example 6 were applied in this Example. Average total Hb levels and average gains in Hb levels (ΔHb) in peripheral blood were measured in thalassemic mice transfected with vectors for 6-week, 5-month, or 9-month. ΔHb was normalized to vector copy (VCN), and ΔHb=Hb level −7.5 (baseline level in thalassemic mice, as all the time points consistently show an HGB value of 7.5 g/dL in the Thalassemic mice used as controls, this value was used). As shown in FIG. 19 and Table 2, globin productions from all vectors, including SNS23.2.B87A1, were stable over time. As shown in FIG. 20, SNS23.2.B87.A1 exhibited equal or better output of β-globin relative to TNS9.B87.A1, especially at late time points.

TABLE 2
Representative data for three different time points after transplant

| | | HGB | Δ(HGB- | ΔHGB/ |
|---|---|---|---|---|
| Vectors | VCN | (g/dL) | 7.5) | copy |
| TNS9.B87.A1 (6 weeks) | 0.5 | 10.6 | 3.8 | 8.6 |
| TNS9.B87.A1 (5 months) | 0.5 | 10.7 | 3.2 | 5.1 |
| TNS9.B87.A1 (9 months) | 0.4 | 9.8 | 2.3 | 6.4 |
| SNS23.2.B87.A1 (6 weeks) | 1.1 | 13.2 | 5.7 | 6.0 |
| SNS23.2.B87.A1 (5 months) | 1.1 | 11.3 | 3.8 | 4.3 |
| SNS23.2.B87.A1 (9 months) | 0.5 | 11.6 | 4.1 | 8.3 |
| SNS26.B87.A1 (6 weeks) | 2.0 | 11.2 | 3.7 | 2.0 |
| SNS26.B87.A1 (5 months) | 3.0 | 11.1 | 3.6 | 1.5 |
| SNS26.B87.A1 (9 months) | 3.1 | 11.1 | 3.6 | 1.4 |
| SNS27.2.B87.A1 (6 weeks) | 0.8 | 11.8 | 4.3 | 5.3 |
| SNS27.2.B87.A1 (5 months) | 1.5 | 12.1 | 4.6 | 3.5 |
| SNS27.2.B87.A1 (9 months) | 1.1 | 10.4 | 2.7 | 2.4 |
| TH3/+ MOCK (6 weeks) | 0 | 7.5 | 0 | 0 |
| TH3/+ MOCK (5 months) | 0 | 7.5 | 0 | 0 |
| TH3/+ MOCK (9 months) | 0 | 7.5 | 0 | 0 |

Average of Vector copy number (VCN) in long-term hematopoietic chimeras. Average Hb level [g/dL] in peripheral blood (PB) of chimeric mice. ΔHGB level was obtained by subtracting Th3/+ hemoglobin (value = 7.5 g/dL) from the total Hb level for each animal tested. ΔHGB/copy = Correlation between delta(Δ)Hb and vector copy number.

All the ΔHGb calculations are made using TH3/+MOCK (7.5 g/dL) as basal HGB, value that is consistent in all the time points.

### Example 8: Human Primary CD34+ Cells Transduced with Vectors Disclosed Herein

Human primary CD34+ cells were isolated by centrifugation on a gradient of Ficoll-Hypaque Plus density. CD34+ cells were purified by positive selection using separation columns and beads. After one day of cytokine stimulation the CD34+ cells were transduced with SNS.23.2.B87.A1 or TNS9.B87.A1 vectors disclosed herein using 4 different MOI. 10 and 15 days after transduction, the cells were harvested and the Vector copy number (VCN) was measured using the methods disclosed in Example 6. As shown in Table 3, at MOIs from 20×1 to 100×1, a linearly increased VCN response was observed in cells transduced with SNS.23.2.B87.A1 vector, with maximum VCN>2. By contrast, cells transduced with TNS9.B87.A1 vector did not exhibit proportional increases in VCN at MOIs from 20×1 to 100×1. Unlike TNS9.B87.A1 vector, the SNS23.2.B87.A1 vector resulted in an increased globin gene transduction in CD34+ cells up to >2 VCN per cell (2.33 at MOI 100, day 15, Table 3).

TABLE 3
Transduction of human primary CD34+ cells

| Groups | MOI | VCN day10 | VCN day15 |
|---|---|---|---|
| SNS23.2.B87.A1 | 10 × 1 | 1 | 0.21 |
| Concentrate virus | 20 × 1 | 1.78 | 0.56 |
| | 40 × 1 | 3.35 | 0.86 |
| | 100 × 1 | 7.33 | 2.33 |
| TNS9.B87.A1 | 10 × 1 | 0.47 | 0.38 |
| Concentrate virus | 20 × 1 | 0.52 | 0.44 |
| | 40 × 1 | 0.48 | 0.40 |
| | 100 × 1 | 0.60 | 0.44 |
| UT | NA | 0.00 | 0.00 |

### REFERENCES

1. Weatherall, D. J. & Clegg, J. B. The Thalassemia Syndrome. Blackwell Scientific Oxford (1981).
2. Stamatoyannopoulos, G., Nienhuis, A. W., Majerus, P. & Varmus, H. The Molecular Basis of Blood Diseaes. WB Saunders, Philadelfphia (1994).
3. Weatherall, D. J. Phenotype-genotype relationships in monogenic disease: lessons from the thalassaemias. Nat Rev Genet 2, 245-255. (2001).
4. Steinberg, M. H., Forget, B. G., Higgs, D. R. & Nagel, R. L. Molecular Mechanism of β Thalassemia; Bernard G. Forget, (Cambridge University Press, Cambridge, UK, 2001).
5. Cooley, T. B. & Lee, P. A series of cases of splenomegaly in children with anemia and peculiar bone changes. Trans. Am. Pediatr. Soc. 37, 29 (1925).
6. Giardina, P. J. & Grady, R. W. Chelation therapy in beta-thalassemia: an optimistic update. Semin Hematol 38, 360-366. (2001).
7. Giardini, C. & Lucarelli, G. Bone marrow transplantation in the treatment of thalassemia. Current opinion in hematology 1, 170-176. (1994).
8 Boulad, F., Giardina, P., Gillio, A., Kernan, N., Small, T., Brochstein, J., Van Syckle, K., George, D., Szabolcs, P. & O'Reilly, R. J. Bone marrow transplantation for homozygous beta-thalassemia. The Memorial Sloan-Kettering Cancer Center experience. Ann N Y Acad Sci 850, 498-502. (1998).
9. Lucarelli, G., Clift, R. A., Galimberti, M., Angelucci, E., Giardini, C., Baronciani, D., Polchi, P., Andreani, M., Gaziev, D., Erer, B., Ciaroni, A., D'Adamo, F., Albertini, F. & Muretto, P. Bone marrow transplantation in adult thalassemic patients. Blood 93, 1164-1167. (1999).
10. Tisdale, J. & Sadelain, M. Toward gene therapy for disorders of globin synthesis. Semin Hematol 38, 382-392 (2001).
11. Pauling, L., Itano, H. A., Singer, S. J. & Wells, I. C. Sickle cell anemia, a molecular disease. Science 110, 543-546 (1949).
12. Swank, R. A. & Stamatoyannopoulos, G. Fetal gene reactivation. Curr Opin Genet Dev 8, 366-370 (1998).
13. Platt, O. S., Orkin, S. H., Dover, G., Beardsley, G. P., Miller, B. & Nathan, D. G. Hydroxyurea enhances fetal hemoglobin production in sickle cell anemia. J Clin Invest 74, 652-656. (1984).
14. Charache, S., Dover, G. J., Moore, R. D., Eckert, S., Ballas, S. K., Koshy, M., Milner, P. F., Orringer, E. P., Phillips, G., Jr., Platt, O. S. & et al. Hydroxyurea: effects on hemoglobin F production in patients with sickle cell anemia. Blood 79, 2555-2565. (1992).
15. Atweh, G. F. & Loukopoulos, D. Pharmacological induction of fetal hemoglobin in sickle cell disease and beta-thalassemia. Semin Hematol 38, 367-373. (2001).
16. Perrine, S. P., Castaneda, S. A., Boosalis, M. S., White, G. L., Jones, B. M. & Bohacek, R. Induction of fetal globin in beta-thalassemia: Cellular obstacles and molecular progress. Ann N Y Acad Sci 1054, 257-265 (2005).
17. Stamatoyannopoulos, G. Prospects for developing a molecular cure for thalassemia. Hematology 10 Suppl 1, 255-257 (2005).
18. Vermylen, C., Cornu, G., Ferster, A., Brichard, B., Ninane, J., Ferrant, A., Zenebergh, A., Maes, P., Dhooge, C., Benoit, Y., Beguin, Y., Dresse, M. F. & Sariban, E. Haematopoietic stem cell transplantation for sickle cell anaemia: the first 50 patients transplanted in Belgium. Bone Marrow Transplant 22, 1-6 (1998).
19. Luzzatto, L. & Goodfellow, P. Sickle cell anaemia. A simple disease with no cure. Nature 337, 17-18 (1989).
20. Sadelain, M. Genetic treatment of the haemoglobinopathies: recombinations and new combinations. Br J Haematol 98, 247-253 (1997).
21. Sadelain, M., Boulad, F., Galanello, R., Giardina, P., Locatelli, F., Maggio, A., Rivella, S., Riviere, I. & Tisdale, J. Therapeutic options for patients with severe beta-thalassemia: the need for globin gene therapy. Hum Gene Ther 18, 1-9 (2007).
22 Borgna-Pignatti, C., Rugolotto, S., De Stefano, P., Zhao, H., Cappellini, M. D., Del Vecchio, G. C., Romeo, M. A., Forni, G. L., Gamberini, M. R., Ghilardi, R., Piga, A. & Cnaan, A. Survival and complications in patients with thalassemia major treated with transfusion and deferoxamine. Haematologica 89, 1187-1193 (2004).
23 Telfer, P. T., Warburton, F., Christou, S., Hadjigavriel, M., Sitarou, M., Kolnagou, A. & Angastiniotis, M. Improved survival in thalassemia major patients on switching from desferrioxamine to combined chelation therapy with desferrioxamine and deferiprone. Haematologica 94, 1777-1778 (2009).
24 Ladis, V., Chouliaras, G., Berdoukas, V., Chatziliami, A., Fragodimitri, C., Karabatsos, F., Youssef, J., Kattamis, A. & Karagiorga-Lagana, M. Survival in a large cohort of Greek patients with transfusion-dependent beta thalassaemia and mortality ratios compared to the general population. European journal of haematology 86, 332-338 (2011).
25. Mancuso, A., Sciarrino, E., Renda, M. C. & Maggio, A. A prospective study of hepatocellular carcinoma incidence in thalassemia. Hemoglobin 30, 119-124 (2006).
26. Persons, D. A. & Tisdale, J. F. Gene therapy for the hemoglobin disorders. Semin Hematol 41, 279-286 (2004).
27. Sadelain, M. Recent advances in globin gene transfer for the treatment of beta-thalassemia and sickle cell anemia. Current opinion in hematology 13, 142-148 (2006).
28 May, C., Rivella, S., Callegari, J., Heller, G., Gaensler, K. M., Luzzatto, L. & Sadelain, M. Therapeutic haemoglobin synthesis in beta-thalassaemic mice expressing lentivirus-encoded human beta-globin. Nature 406, 82-86 (2000).
29. May, C., Rivella, S., Chadburn, A. & Sadelain, M. Successful treatment of murine beta-thalassemia intermedia by transfer of the human beta-globin gene. Blood 99, 1902-1908 (2002).
30. Rivella, S., May, C., Chadburn, A., Riviere, I. & Sadelain, M. A novel murine model of Cooley anemia and its rescue by lentiviral-mediated human beta-globin gene transfer. Blood 101, 2932-2939 (2003).
31 Sadelain, M., Boulad, F., Lisowki, L., Moi, P. & Riviere, I. Stem cell engineering for the treatment of severe hemoglobinopathies. Curr Mol Med 8, 690-697 (2008).
32. Bank, A., Dorazio, R. & Leboulch, P. A phase I/II clinical trial of beta-globin gene therapy for beta-thalassemia. Ann N Y Acad Sci 1054, 308-316 (2005).
33. Cavazzana-Calvo, M., Payen, E., Negre, O., Wang, G., Hehir, K., Fusil, F., Down, J., Denaro, M., Brady, T., Westerman, K., Cavallesco, R., Gillet-Legrand, B., Caccavelli, L., Sgarra, R., Maouche-Chretien, L., Bernaudin, F., Girot, R., Dorazio, R., Mulder, G. J., Polack, A., Bank, A., Soulier, J., Larghero, J., Kabbara, N., Dalle, B., Gourmel, B., Socie, G., Chretien, S., Cartier, N., Aubourg, P., Fischer, A., Cornetta, K., Galacteros, F., Beuzard, Y., Gluckman, E., Bushman, F., Hacein-Bey-Abina, S. & Leboulch, P. Transfusion independence and HMGA2 activation after gene therapy of human beta-thalassaemia. Nature 467, 318-322 (2010).
34. Braun, C. J., Boztug, K., Paruzynski, A., Witzel, M., Schwarzer, A., Rothe, M., Modlich, U., Beier, R., Gohring, G., Steinemann, D., Fronza, R., Ball, C. R., Haemmerle, R., Naundorf, S., Kuhlcke, K., Rose, M., Fraser, C., Mathias, L., Ferrari, R., Abboud, M. R., Al-Herz, W., Kondratenko, I., Marodi, L., Glimm, H., Schlegelberger, B., Schambach, A., Albert, M. H., Schmidt, M., von Kalle, C. & Klein, C. Gene therapy for Wiskott-Aldrich syndrome—long-term efficacy and genotoxicity. Sci Transl Med 6, 227ra233 (2014).
35. Chang, A. H. & Sadelain, M. The genetic engineering of hematopoietic stem cells: the rise of lentiviral vectors, the conundrum of the ltr, and the promise of lineage-restricted vectors. Mol Ther 15, 445-456 (2007).
36. Pawliuk, R., Westerman, K. A., Fabry, M. E., Payen, E., Tighe, R., Bouhassira, E. E., Acharya, S. A., Ellis, J., London, I. M., Eaves, C. J., Humphries, R. K., Beuzard, Y., Nagel, R. L. & Leboulch, P. Correction of sickle cell disease in transgenic mouse models by gene therapy. Science 294, 2368-2371 (2001).
37. Emery, D. W., Chen, H., Li, Q. & Stamatoyannopoulos, G. Development of a condensed locus control region cassette and testing in retrovirus vectors for A gamma-globin. Blood Cells Mol Dis 24, 322-339 (1998).
38. Miccio, A., Cesari, R., Lotti, F., Rossi, C., Sanvito, F., Ponzoni, M., Routledge, S. J., Chow, C. M., Antoniou, M. N. & Ferrari, G. In vivo selection of genetically modified erythroblastic progenitors leads to long-term correction of beta-thalassemia. Proc Natl Acad Sci USA 105, 10547-10552 (2008).
39. Sadelain, M., Wang, C. H., Antoniou, M., Grosveld, F. & Mulligan, R. C. Generation of a high-titer retroviral vector capable of expressing high levels of the human beta-globin gene. Proc Natl Acad Sci USA 92, 6728-6732 (1995).
40. Samakoglu, S., Lisowski, L., Budak-Alpdogan, T., Usachenko, Y., Acuto, S., Di Marzo, R., Maggio, A., Zhu, P., Tisdale, J. F., Riviere, I. & Sadelain, M. A genetic strategy to treat sickle cell anemia by coregulating globin transgene expression and RNA interference. Nat Biotechnol 24, 89-94 (2006).
41. Pestina, T. I., Hargrove, P. W., Jay, D., Gray, J. T., Boyd, K. M. & Persons, D. A. Correction of murine sickle cell disease using gamma-globin lentiviral vectors to mediate high-level expression of fetal hemoglobin. Mol Ther 17, 245-252 (2009).
42. Hanawa, H., Yamamoto, M., Zhao, H., Shimada, T. & Persons, D. A. Optimized lentiviral vector design improves titer and transgene expression of vectors containing the chicken beta-globin locus HS4 insulator element. Mol Ther 17, 667-674 (2009).
43. Arumugam, P. I., Scholes, J., Perelman, N., Xia, P., Yee, J. K. & Malik, P. Improved human beta-globin expression from self-inactivating lentiviral vectors carrying the chicken hypersensitive site-4 (cHS4) insulator element. Mol Ther 15, 1863-1871 (2007).
44. Fraser, P., Pruzina, S., Antoniou, M. & Grosveld, F. Each hypersensitive site of the human beta-globin locus control region confers a different developmental pattern of expression on the globin genes. Genes & development 7, 10⁶-113 (1993).
45. Navas, P. A., Peterson, K. R., Li, Q., Skarpidi, E., Rohde, A., Shaw, S. E., Clegg, C. H., Asano, H. & Stamatoyannopoulos, G. Developmental specificity of the interaction between the locus control region and embryonic or fetal globin genes in transgenic mice with an HS3 core deletion. Molecular and cellular biology 18, 418δ-4196 (1998).
46. Li, Q. & Stamatoyannopoulos, G. Hypersensitive site 5 of the human beta locus control region functions as a chromatin insulator. Blood 84, 1399-1401 (1994).
47. Li, Q., Zhang, M., Han, H., Rohde, A. & Stamatoyannopoulos, G. Evidence that DNase I hypersensitive site 5 of the human beta-globin locus control region functions as a chromosomal insulator in transgenic mice. Nucleic Acids Res 30, 2484-2491 (2002).
48. Puthenveetil, G., Scholes, J., Carbonell, D., Qureshi, N., Xia, P., Zeng, L., Li, S., Yu, Y., Hiti, A. L., Yee, J. K. & Malik, P. Successful correction of the human beta-thalassemia major phenotype using a lentiviral vector. Blood 104, 3445-3453 (2004).
49. Wilber, A., Nienhuis, A. W. & Persons, D. A. Transcriptional regulation of fetal to adult hemoglobin switching: new therapeutic opportunities. Blood 117, 3945-3953 (2011).
50. Arumugam, P. I., Higashimoto, T., Urbinati, F., Modlich, U., Nestheide, S., Xia, P., Fox, C., Corsinotti, A., Baum, C. & Malik, P. Genotoxic potential of lineage-specific lentivirus vectors carrying the beta-globin locus control region. Mol Ther 17, 1929-1937 (2009).
51. Chang, K. H., Fang, X., Wang, H., Huang, A., Cao, H., Yang, Y., Bonig, H., Stamatoyannopoulos, J. A. & Papayannopoulou, T. Epigenetic modifications and chromosome conformations of the beta globin locus throughout development. Stem cell reviews 9, 397-407 (2013).
52. Papayannopoulou, T., Priestley, G. V., Rohde, A., Peterson, K. R. & Nakamoto, B. Hemopoietic lineage commitment decisions: in vivo evidence from a transgenic mouse model harboring micro LCR-betapro-LacZ as a transgene. Blood 95, 1274-1282 (2000).
53. Nienhuis, A. W. Development of gene therapy for blood disorders: an update. Blood 122, 1556-1564 (2013).
54 Baum, C., Kustikova, O., Modlich, U., Li, Z. & Fehse, B. Mutagenesis and oncogenesis by chromosomal insertion of gene transfer vectors. Hum Gene Ther 17, 253-263 (2006).
55. Nienhuis, A. W., Dunbar, C. E. & Sorrentino, B. P. Genotoxicity of retroviral integration in hematopoietic cells. Mol Ther 13, 1031-1049 (2006).
56. Emery, D. W. The use of chromatin insulators to improve the expression and safety of integrating gene transfer vectors. Hum Gene Ther 22, 761-774 (2011).
57. Evans-Galea, M. V., Wielgosz, M. M., Hanawa, H., Srivastava, D. K. & Nienhuis, A. W. Suppression of clonal dominance in cultured human lymphoid cells by addition of the cHS4 insulator to a lentiviral vector. Mol Ther 15, 801-809 (2007).
58. Rivella, S., Callegari, J. A., May, C., Tan, C. W. & Sadelain, M. The cHS4 insulator increases the probability of retroviral expression at random chromosomal integration sites. J Virol 74, 4679-4687 (2000).
59 Emery, D. W., Yannaki, E., Tubb, J. & Stamatoyannopoulos, G. A chromatin insulator protects retrovirus vectors from chromosomal position effects. Proc Natl Acad Sci USA 97, 9150-9155 (2000).
60. Emery, D. W., Yannaki, E., Tubb, J., Nishino, T., Li, Q. & Stamatoyannopoulos, G. Development of virus vectors for gene therapy of beta chain hemoglobinopathies: flanking with a chromatin insulator reduces gamma-globin gene silencing in vivo. Blood 100, 2012-2019 (2002).
61. Yannaki, E., Tubb, J., Aker, M., Stamatoyannopoulos, G. & Emery, D. W. Topological constraints governing the use of the chicken HS4 chromatin insulator in oncoretrovirus vectors. Mol Ther 5, 589-598 (2002).
62 Hino, S., Fan, J., Taguwa, S., Akasaka, K. & Matsuoka, M. Sea urchin insulator protects lentiviral vector from silencing by maintaining active chromatin structure. Gene Ther 11, 819-828 (2004).
63 Ramezani, A., Hawley, T. S. & Hawley, R. G. Performance- and safety-enhanced lentiviral vectors containing the human interferon-beta scaffold attachment region and the chicken beta-globin insulator. Blood 101, 4717-4724 (2003).
64. Ramezani, A., Hawley, T. S. & Hawley, R. G. Combinatorial incorporation of enhancer-blocking components of the chicken beta-globin 5′HS4 and human T-cell receptor alpha/delta BEAD-1 insulators in self-inactivating retroviral vectors reduces their genotoxic potential. Stem Cells 26, 3257-3266 (2008).
65. Yannaki, E., Emery, D. W. & Stamatoyannopoulos, G. Gene therapy for beta-thalassaemia: the continuing challenge. Expert reviews in molecular medicine 12, e31 (2010).
66. Persons, D. A. The challenge of obtaining therapeutic levels of genetically modified hematopoietic stem cells in beta-thalassemia patients. Ann N Y Acad Sci 1202, 69-74 (2010).
67. Perumbeti, A. & Malik, P. Therapy for beta-globinopathies: a brief review and determinants for successful and safe correction. Ann N Y Acad Sci 1202, 36-44 (2010).
68. Johnson, K. D., Grass, J. A., Park, C., Im, H., Choi, K. & Bresnick, E. H. Highly restricted localization of RNA polymerase II within a locus control region of a tissue-specific chromatin domain. Molecular and cellular biology 23, 6484-6493 (2003).
69. Vieira, K. F., Levings, P. P., Hill, M. A., Crusselle, V. J., Kang, S. H., Engel, J. D. & Bungert, J. Recruitment of transcription complexes to the beta-globin gene locus in vivo and in vitro. J Biol Chem 279, 50350-50357 (2004).
70. Levings, P. P., Zhou, Z., Vieira, K. F., Crusselle-Davis, V. J. & Bungert, J. Recruitment of transcription complexes to the beta-globin locus control region and transcription of hypersensitive site 3 prior to erythroid differentiation of murine embryonic stem cells. The FEBS journal 273, 746-755 (2006).
71 Felsenfeld, G. & Groudine, M. Controlling the double helix. Nature 421, 448-453 (2003).
72. Felsenfeld, G. Chromatin as an essential part of the transcriptional mechanism. Nature 355, 219-224 (1992).
73. Brownell, J. E. & Allis, C. D. Special HATs for special occasions: linking histone acetylation to chromatin assembly and gene activation. Curr Opin Genet Dev 6, 176-184 (1996).
74 Kingston, R. E. & Narlikar, G. J. ATP-dependent remodeling and acetylation as regulators of chromatin fluidity. Genes & development 13, 2339-2352 (1999).
75 Tsukiyama, T. & Wu, C. Chromatin remodeling and transcription. Curr Opin Genet Dev 7, 182-191 (1997).
76. Wolffe, A. P., Wong, J. & Pruss, D. Activators and repressors: making use of chromatin to regulate transcription. Genes to cells: devoted to molecular & cellular mechanisms 2, 291-302 (1997).
77. Kadonaga, J. T. Eukaryotic transcription: an interlaced network of transcription factors and chromatin-modifying machines. Cell 92, 307-313 (1998).
78 Struhl, K. Histone acetylation and transcriptional regulatory mechanisms. Genes & development 12, 599-606 (1998).
79. Gross, D. S. & Garrard, W. T. Nuclease hypersensitive sites in chromatin. Annual review of biochemistry 57, 159-197 (1988).
80. Elgin, S. C. Anatomy of hypersensitive sites. Nature 309, 213-214 (1984).
81. Wu, C. The 5′ ends of Drosophila heat shock genes in chromatin are hypersensitive to DNase I. Nature 286, 854-860 (1980).
82. Felsenfeld, G., Boyes, J., Chung, J., Clark, D. & Studitsky, V. Chromatin structure and gene expression. Proc Natl Acad Sci USA 93, 9384-9388 (1996).
83. Burgess-Beusse, B., Farrell, C., Gaszner, M., Litt, M., Mutskov, V., Recillas-Targa, F., Simpson, M., West, A. & Felsenfeld, G. The insulation of genes from external enhancers and silencing chromatin. Proc Natl Acad Sci USA 99 Suppl 4, 16433-16437 (2002).
84. Elgin, S. C. DNAase I-hypersensitive sites of chromatin. Cell 27, 413-415 (1981).
85 McGhee, J. D., Wood, W. I., Dolan, M., Engel, J. D. & Felsenfeld, G. A 200 base pair region at the 5′ end of the chicken adult beta-globin gene is accessible to nuclease digestion. Cell 27, 45-55 (1981).
86. Lowrey, C. H., Bodine, D. M. & Nienhuis, A. W. Mechanism of DNase I hypersensitive site formation within the human globin locus control region. Proc Natl Acad Sci USA 89, 1143-1147 (1992).
87. Adams, C. C. & Workman, J. L. Binding of disparate transcriptional activators to nucleosomal DNA is inherently cooperative. Molecular and cellular biology 15, 1405-1421 (1995).
88. McArthur, M., Gerum, S. & Stamatoyannopoulos, G. Quantification of DNasel-sensitivity by real-time PCR: quantitative analysis of DNasel-hypersensitivity of the mouse beta-globin LCR. J Mol Biol 313, 27-34 (2001).
89. Dorschner, M. O., Hawrylycz, M., Humbert, R., Wallace, J. C., Shafer, A., Kawamoto, J., Mack, J., Hall, R., Goldy, J., Sabo, P. J., Kohli, A., Li, Q., McArthur, M. & Stamatoyannopoulos, J. A. High-throughput localization of functional elements by quantitative chromatin profiling. Nat Methods 1, 219-225 (2004).
90. Sabo, P. J., Kuehn, M. S., Thurman, R., Johnson, B. E., Johnson, E. M., Cao, H., Yu, M., Rosenzweig, E., Goldy, J., Haydock, A., Weaver, M., Shafer, A., Lee, K., Neri, F., Humbert, R., Singer, M. A., Richmond, T. A., Dorschner, M. O., McArthur, M., Hawrylycz, M., Green, R. D., Navas, P. A., Noble, W. S. & Stamatoyannopoulos, J. A. Genome-scale mapping of DNase I sensitivity in vivo using tiling DNA microarrays. Nat Methods 3, 511-518 (2006).
91. Sabo, P. J., Hawrylycz, M., Wallace, J. C., Humbert, R., Yu, M., Shafer, A., Kawamoto, J., Hall, R., Mack, J., Dorschner, M. O., McArthur, M. & Stamatoyannopoulos, J. A. Discovery of functional noncoding elements by digital analysis of chromatin structure. Proc Natl Acad Sci USA 101, 16837-16842 (2004).
92. Sabo, P. J., Humbert, R., Hawrylycz, M., Wallace, J. C., Dorschner, M. O., McArthur, M. & Stamatoyannopoulos, J. A. Genome-wide identification of DNasel hypersensitive sites using active chromatin sequence libraries. Proc Natl Acad Sci USA 101, 4537-4542 (2004).
93. Thurman, R. E., Rynes, E., Humbert, R., Vierstra, J., Maurano, M. T., Haugen, E., Sheffield, N. C., Stergachis, A. B., Wang, H., Vernot, B., Garg, K., John, S., Sandstrom, R., Bates, D., Boatman, L., Canfield, T. K., Diegel, M., Dunn, D., Ebersol, A. K., Frum, T., Giste, E., Johnson, A. K., Johnson, E. M., Kutyavin, T., Lajoie, B., Lee, B. K., Lee, K., London, D., Lotakis, D., Neph, S., Neri, F., Nguyen, E. D., Qu, H., Reynolds, A. P., Roach, V., Safi, A., Sanchez, M. E., Sanyal, A., Shafer, A., Simon, J. M., Song, L., Vong, S., Weaver, M., Yan, Y., Zhang, Z., Zhang, Z., Lenhard, B., Tewari, M., Dorschner, M. O., Hansen, R. S., Navas, P. A., Stamatoyannopoulos, G., Iyer, V. R., Lieb, J. D., Sunyaev, S. R., Akey, J. M., Sabo, P. J., Kaul, R., Furey, T. S., Dekker, J., Crawford, G. E. & Stamatoyannopoulos, J. A. The accessible chromatin landscape of the human genome. Nature 489, 75-82 (2012).
94. Stergachis, A. B., Neph, S., Reynolds, A., Humbert, R., Miller, B., Paige, S. L., Vernot, B., Cheng, J. B., Thurman, R. E., Sandstrom, R., Haugen, E., Heimfeld, S., Murry, C. E., Akey, J. M. & Stamatoyannopoulos, J. A. Developmental fate and cellular maturity encoded in human regulatory DNA landscapes. Cell 154, 88δ-903 (2013).
95. Neph, S., Stergachis, A. B., Reynolds, A., Sandstrom, R., Borenstein, E. & Stamatoyannopoulos, J. A. Circuitry and dynamics of human transcription factor regulatory networks. Cell 150, 1274-1286 (2012).
96. Maurano, M. T., Humbert, R., Rynes, E., Thurman, R. E., Haugen, E., Wang, H., Reynolds, A. P., Sandstrom, R., Qu, H., Brody, J., Shafer, A., Neri, F., Lee, K., Kutyavin, T., Stehling-Sun, S., Johnson, A. K., Canfield, T. K., Giste, E., Diegel, M., Bates, D., Hansen, R. S., Neph, S., Sabo, P. J., Heimfeld, S., Raubitschek, A., Ziegler, S., Cotsapas, C., Sotoodehnia, N., Glass, I., Sunyaev, S. R., Kaul, R. & Stamatoyannopoulos, J. A. Systematic localization of common disease-associated variation in regulatory DNA. Science 337, 1190-1195 (2012).
97. Stergachis, A. B., Haugen, E., Shafer, A., Fu, W., Vernot, B., Reynolds, A., Raubitschek, A., Ziegler, S., LeProust, E. M., Akey, J. M. & Stamatoyannopoulos, J. A. Exonic transcription factor binding directs codon choice and affects protein evolution. Science 342, 1367-1372 (2013).
98. Neph, S., Vierstra, J., Stergachis, A. B., Reynolds, A. P., Haugen, E., Vernot, B., Thurman, R. E., John, S., Sandstrom, R., Johnson, A. K., Maurano, M. T., Humbert, R., Rynes, E., Wang, H., Vong, S., Lee, K., Bates, D., Diegel, M., Roach, V., Dunn, D., Neri, J., Schafer, A., Hansen, R. S., Kutyavin, T., Giste, E., Weaver, M., Canfield, T., Sabo, P., Zhang, M., Balasundaram, G., Byron, R., MacCoss, M. J., Akey, J. M., Bender, M. A., Groudine, M., Kaul, R. & Stamatoyannopoulos, J. A. An expansive human regulatory lexicon encoded in transcription factor footprints. Nature 489, 83-90 (2012).
99. Ramezani, A., Hawley, T. S. & Hawley, R. G. Stable gammaretroviral vector expression during embryonic stem cell-derived in vitro hematopoietic development. Mol Ther 14, 245-254 (2006).
100. Recillas-Targa, F., Pikaart, M. J., Burgess-Beusse, B., Bell, A. C., Litt, M. D., West, A. G., Gaszner, M. & Felsenfeld, G. Position-effect protection and enhancer blocking by the chicken beta-globin insulator are separable activities. Proc Natl Acad Sci USA 99, 6883-6888 (2002).
101. Gaszner, M. & Felsenfeld, G. Insulators: exploiting transcriptional and epigenetic mechanisms. Nat Rev Genet 7, 703-713 (2006).
102. Wallace, J. A. & Felsenfeld, G. We gather together: insulators and genome organization. Curr Opin Genet Dev 17, 400-407 (2007).
103. Chung, J. H., Bell, A. C. & Felsenfeld, G. Characterization of the chicken beta-globin insulator. Proc Natl Acad Sci USA 94, 575-580 (1997).
104. Bell, A. C., West, A. G. & Felsenfeld, G. The protein CTCF is required for the enhancer blocking activity of vertebrate insulators. Cell 98, 387-396 (1999).
105. Ryu, B. Y., Persons, D. A., Evans-Galea, M. V., Gray, J. T. & Nienhuis, A. W. A chromatin insulator blocks interactions between globin regulatory elements and cellular promoters in erythroid cells. Blood Cells Mol Dis 39, 221-228 (2007).
10⁶. Ryu, B. Y., Evans-Galea, M. V., Gray, J. T., Bodine, D. M., Persons, D. A. & Nienhuis, A. W. An experimental system for the evaluation of retroviral vector design to diminish the risk for proto-oncogene activation. Blood 111, 1866-1875 (2008).
107. Yao, S., Osborne, C. S., Bharadwaj, R. R., Pasceri, P., Sukonnik, T., Pannell, D., Recillas-Targa, F., West, A. G. & Ellis, J. Retrovirus silencer blocking by the cHS4 insulator is CTCF independent. Nucleic Acids Res 31, 5317-5323 (2003).
108. Nishino, T., Tubb, J. & Emery, D. W. Partial correction of murine beta-thalassemia with a gammaretrovirus vector for human gamma-globin. Blood Cells Mol Dis 37, 1-7 (2006).
109. Aker, M., Tubb, J., Groth, A. C., Bukovsky, A. A., Bell, A. C., Felsenfeld, G., Kiem, H. P., Stamatoyannopoulos, G. & Emery, D. W. Extended core sequences from the cHS4 insulator are necessary for protecting retroviral vectors from silencing position effects. Hum Gene Ther 18, 333-343 (2007).
110. Li, C. L. & Emery, D. W. The cHS4 chromatin insulator reduces gammaretroviral vector silencing by epigenetic modifications of integrated provirus. Gene Ther 15, 49-53 (2008).
111. Ma, Y., Ramezani, A., Lewis, R., Hawley, R. G. & Thomson, J. A. High-level sustained transgene expression in human embryonic stem cells using lentiviral vectors. Stem Cells 21, 111-117 (2003).
112. Chang, L. J., Liu, X. & He, J. Lentiviral siRNAs targeting multiple highly conserved RNA sequences of human immunodeficiency virus type 1. Gene Ther 12, 1133-1144 (2005).
113. Pluta, K., Luce, M. J., Bao, L., Agha-Mohammadi, S. & Reiser, J. Tight control of transgene expression by lentivirus vectors containing second-generation tetracycline-responsive promoters. J Gene Med 7, 803-817 (2005).
114. Jakobsson, J., Rosenqvist, N., Thompson, L., Barraud, P. & Lundberg, C. Dynamics of transgene expression in a neural stem cell line transduced with lentiviral vectors incorporating the cHS4 insulator. Experimental cell research 298, 611-623 (2004).
115. Leboulch, P., Huang, G. M., Humphries, R. K., Oh, Y. H., Eaves, C. J., Tuan, D. Y. & London, I. M. Mutagenesis of retroviral vectors transducing human beta-globin gene and beta-globin locus control region derivatives results in stable transmission of an active transcriptional structure. EMBO J 13, 3065-3076 (1994).
116. Kim, T. H., Abdullaev, Z. K., Smith, A. D., Ching, K. A., Loukinov, D. I., Green, R. D., Zhang, M. Q., Lobanenkov, V. V. & Ren, B. Analysis of the vertebrate insulator protein CTCF-binding sites in the human genome. Cell 128, 1231-1245 (2007).
117. Yusufzai, T. M. & Felsenfeld, G. The 5′-HS4 chicken beta-globin insulator is a CTCF-dependent nuclear matrix-associated element. Proc Natl Acad Sci USA 101, 8620-8624 (2004).
118. Phillips, J. E. & Corces, V. G. CTCF: master weaver of the genome. Cell 137, 1194-1211 (2009).
119. Giles, K. E., Gowher, H., Ghirlando, R., Jin, C. & Felsenfeld, G. Chromatin boundaries, insulators, and long-range interactions in the nucleus. Cold Spring Harbor symposia on quantitative biology 75, 79-85 (2010).
120. Barski, A., Cuddapah, S., Cui, K., Roh, T. Y., Schones, D. E., Wang, Z., Wei, G., Chepelev, I. & Zhao, K. High-resolution profiling of histone methylations in the human genome. Cell 129, 823-837 (2007).
121. Wang, H., Maurano, M. T., Qu, H., Varley, K. E., Gertz, J., Pauli, F., Lee, K., Canfield, T., Weaver, M., Sandstrom, R., Thurman, R. E., Kaul, R., Myers, R. M. & Stamatoyannopoulos, J. A. Widespread plasticity in CTCF occupancy linked to DNA methylation. Genome research 22, 1680-1688 (2012).
122. Schmidt, D., Schwalie, P. C., Wilson, M. D., Ballester, B., Goncalves, A., Kutter, C., Brown, G. D., Marshall, A., Flicek, P. & Odom, D. T. Waves of retrotransposon expansion remodel genome organization and CTCF binding in multiple mammalian lineages. Cell 148, 335-348 (2012).
123. Renda, M., Baglivo, I., Burgess-Beusse, B., Esposito, S., Fattorusso, R., Felsenfeld, G. & Pedone, P. V. Critical DNA binding interactions of the insulator protein CTCF: a small number of zinc fingers mediate strong binding, and a single finger-DNA interaction controls binding at imprinted loci. J Biol Chem 282, 33336-33345 (2007).
124. Dickson, J., Gowher, H., Strogantsev, R., Gaszner, M., Hair, A., Felsenfeld, G. & West, A. G. VEZF1 elements mediate protection from DNA methylation. PLOS Genet 6, e1000804 (2010).
125. Li, C. L., Xiong, D., Stamatoyannopoulos, G. & Emery, D. W. Genomic and functional assays demonstrate reduced gammaretroviral vector genotoxicity associated with use of the cHS4 chromatin insulator. Mol Ther 17, 716-724 (2009).
126. Lisowski, L. & Sadelain, M. Locus control region elements HS1 and HS4 enhance the therapeutic efficacy of globin gene transfer in beta-thalassemic mice. Blood 110, 4175-4178 (2007).
127. Nagel, R. L., Bookchin, R. M., Johnson, J., Labie, D., Wajcman, H., Isaac-Sodeye, W. A., Honig, G. R., Schiliro, G., Crookston, J. H. & Matsutomo, K. Structural bases of the inhibitory effects of hemoglobin F and hemoglobin A2 on the polymerization of hemoglobin S. Proc Natl Acad Sci USA 76, 670-672 (1979).
128. Sadelain et al., Proc. Nat'l Acad. Sci. (USA) (1995); 92:672δ-6732.
129. Armstrong, J. A., Emerson, B. M., 1996. NFE2 disrupts chromatin structure at human fl-globin locus control region hypersensitive site 2 in vitro. Mol. Cell. Biol. 16, 5634-5644.
130. Caterina, J. J., Ciavatta, D. J., Donze, D., Behringer, R. R., Townes, T. M., 1994. Multiple elements in human fl-globin locus control region 5′ HS2 are involved in enhancer activity and position-independent transgene expression. Nucleic Acids Res. 22, 1006 1011.
131. Moi, P., Kan, Y. W., 1990. Synergistic enhancement of globin gene expression by activator protein-l-like proteins. Proc. Natl. Acad, Sci. USA 87, 9000-9004.
132. Ney, P., Sorrentino, B., McDonagh, K., Nienhuis, A., 1990. Tandem AP-1-binding sites within the human/j-globin dominant control region function as an inducible enhancer in erythroid cells. Genes Dev. 4, 993 1006.
133. Shivdasani, R. A., Rosenblatt, M. F., Zucker-Franklin, D., Jackson, C. W., Hunt, P., Saris, C. J. M., Orkin, S. H., 1995. Transcription factor NF-E2 is required for platelet formation independent of the actions of thrombopoietin/MGDF in megakaryocyte development. Cell 81,695-704.
134. Talbot, D., Grosveld, F., 1991. The 5′HS2 of the globin locus control region enhances transcription through the interaction of a multimeric complex binding at two functionally distinct NF-E2 binding sites. EMBO J. 10, 1391-1398.
135. Hardison et al., Gene (1997); 205:73-94.
136. Elnitski et al., The Journal of Biological Chemistry (1997); 272(1):369-378; Horak et al., PNAS (2002); 99(5):2924-2929.
137. Shimotsuma et al., Journal of Biological Chemistry (2010); 285(19): 14495-14503.

From the foregoing description, it will be apparent that variations and modifications may be made to the presently disclosed subject matter described herein to adopt it to various usages and conditions. Such embodiments are also within the scope of the following claims.

All patents and publications and sequences referred to by accession or reference number mentioned in this specification are herein incorporated by reference to the same extent as if each independent patent and publication and sequence was specifically and individually indicated to be incorporated by reference.

1-67. (canceled)

68. A cell transduced with an expression cassette wherein the expression cassette comprises:
a) a globin gene or a functional portion thereof operably linked to a β-globin locus control region (LCR) that comprises a Dnase I hypersensitive site-2 (HS2) region, a Dnase I hypersensitive site-3 (HS3) region, and a Dnase I hypersensitive site-4 (HS4) region, wherein the HS4 region has a length of less than about 800 bp and does not comprise the nucleotide sequence set forth in SEQ ID NO: 50; or
b) a globin gene or a functional portion thereof operably linked to a β-globin locus control region (LCR) that comprises a HS1 region, a HS3 region, and a HS4 region and does not comprise a core sequence of HS2 region, wherein the HS4 region has a length of less than about 800 bp and does not comprise the nucleotide sequence set forth in SEQ ID NO:50.

69. The cell of claim 68, wherein the β-globin LCR comprises:
a) a HS2 region consisting of nucleotides 45-860 of SEQ ID NO: 9, a HS3 region consisting of the nucleotide sequence set forth in SEQ ID NO: 5, and a HS4 region consisting of nucleotides 115-868 of SEQ ID NO: 6;
b) a HS2 region consisting of the nucleotide sequence set forth in SEQ ID NO: 33, a HS3 region consisting of the nucleotide sequence set forth in SEQ ID NO: 34, and a HS4 region consisting of the nucleotide sequence set forth in SEQ ID NO: 35;
c) a HS1 region consisting of the nucleotide sequence set forth in SEQ ID NO: 3, a HS2 region consisting of nucleotides 45-860 of SEQ ID NO: 9, a HS3 region consisting of the nucleotide sequence set forth in SEQ ID NO: 5, and a HS4 region consisting of nucleotides 115-868 of SEQ ID NO: 6;
d) a HS1 region consisting of the nucleotide sequence set forth in SEQ ID NO: 3, a HS2 region consisting of the nucleotide sequence set forth in SEQ ID NO: 33, a HS3 region consisting of the nucleotide sequence set forth in SEQ ID NO: 34, and a HS4 region consisting of the nucleotide sequence set forth in SEQ ID NO: 35;
e) a HS1 region consisting of the nucleotide sequence set forth in SEQ ID NO: 3, a HS3 region consisting of the nucleotide sequence set forth in SEQ ID NO: 5, and a HS4 region consisting of nucleotides 115-868 of SEQ ID NO: 6; or
f) a HS1 region consisting of the nucleotide sequence set forth in SEQ ID NO: 3, a HS3 region consisting of the nucleotide sequence set forth in SEQ ID NO: 34, and a HS4 region consisting of the nucleotide sequence set forth in SEQ ID NO: 35.

70. The cell of claim 68, wherein the expression cassette, further comprising at least one erythroid-specific enhancer.

71. The cell of claim 70, wherein the at least one erythroid-specific enhancer is positioned within the β-globin LCR.

72. The cell of claim 71, wherein the at least one erythroid-specific enhancer is positioned between the HS1 region and the HS3 region of the β-globin LCR.

73. The cell of claim 70, wherein the at least one erythroid-specific enhancer consists of the nucleotide sequence set forth in SEQ ID NO: 13, SEQ ID NO: 14, SEQ ID NO: 15, SEQ ID NO: 16, or SEQ ID NO: 17.

74. The cell of claim 68, wherein the expression cassette further comprising at least one insulator that comprises the CTCF binding site sequence set forth in SEQ ID NO:18.

75. The cell of claim 74, wherein the at least one insulator consists of the nucleotide sequence set forth in SEQ ID NO: 1, SEQ ID NO: 24, SEQ ID NO: 25, SEQ ID NO: 27, SEQ ID NO: 28, SEQ ID NO: 29, SEQ ID NO: 30, SEQ ID NO: 31, or SEQ ID NO: 32, or a fragment thereof.

76. The cell of claim 68, wherein the human β-globin gene is a wild-type human β-globin gene or a non-wild-type human β-globin gene.

77. The cell of claim 76, wherein non-wild-type human β-globin gene is selected from a human β-globin gene comprising one or more deletions of intron sequences, a human β-globin gene encoding at least one anti-sickling amino acid residue, and a human β-globin gene comprising one or more deletions of intron sequences and encoding at least one anti-sickling amino acid residue.

78. The cell of claim 77, wherein the human β-globin gene encoding at least one anti-sickling amino acid residue is selected from the group consisting of a human β^{A}-globin gene encoding a threonine to glutamine mutation at codon 87 (β^{A-T87Q}), a human β^{A}-globin gene encoding a glutamic acid to alanine mutation at codon 22 (β^{A-E22A}), a human β^{A}-globin gene encoding an asparagine to lysine mutation at codon 80 (β-N80K), a human β^{A}-globin gene encoding a glutamic acid to alanine mutation at codon 22 and an asparagine to lysine mutation at codon 80, and a human β^{A}-globin gene encoding a glutamic acid to alanine mutation at codon 22 and a threonine to glutamine mutation at codon 87.

79. The cell of claim 78, wherein the one or more deletions of intron sequences comprise a deletion in intron 2.

80. The cell of claim 68, wherein the expression cassette further comprises a β-globin promoter.

81. The cell of claim 80, wherein said β-globin promoter is positioned between the globin gene or functional portion thereof and the β-globin LCR.

82. The cell of claim 81, wherein said β-globin promoter is a human β-globin promoter that is about 265 bp in length, and/or consists of the nucleotide sequence set forth in SEQ ID NO:11.

83. The cell of claim 68, wherein the expression cassette further comprises a human β-globin 3′ enhancer.

84. The cell of claim 83, wherein said human β-globin 3′ enhancer is positioned in the upstream of the globin gene or functional portion thereof.

85. The cell of claim 83, wherein said human β-globin 3′ enhancer is about 880 bp in length and/or consists of the nucleotide sequence set forth in SEQ ID NO:12.

86. The cell of claim 68, wherein the cell is selected from the group consisting of a hematopoietic stem cell, an embryonic stem cell, an induced pluripotent stem cell, and a hemogenic endothelium cell, optionally wherein the hematopoietic stem cell is a CD34+ hematopoietic stem cell.

87. A method of treating a hemoglobinopathy in a subject, comprising administering an effective amount of the cell of claim 68 to the subject, thereby restoring the subject's ability to produce red blood cells containing normal hemoglobin.
